(19) United States
(12) Patent Application Publication
(10) Pub. No.: US 2022/0290142 A1
(43) Pub. Date: Sep. 15, 2022

(54) COMPOSITIONS AND METHODS FOR MODULATING SPLICING AND PROTEIN EXPRESSION

(71) Applicant: Stoke Therapeutics, Inc., Bedford, MA (US)

(72) Inventor: Isabel Aznarez, Boston, MA (US)

(21) Appl. No.: 17/673,226

(22) Filed: Feb. 16, 2022

Related U.S. Application Data
(63) Continuation of application No. PCT/US2020/047081, filed on Aug. 19, 2020.
(60) Provisional application No. 63/049,262, filed on Jul. 8, 2020.
(60) Provisional application No. 62/888,887, filed on Aug. 19, 2019.

(51) Int. Cl. C12N 15/113; A61K 31/7125

(52) U.S. Cl. CPC C12N 15/113; A61K 31/7125; C12N 2310/11; C12N 2310/315; C12N 2310/3233; C12N 2310/321; C12N 2320/30; C12N 2310/314; C12N 2310/346

(57) ABSTRACT

Alternative splicing events can lead to non-productive mRNA transcripts which in turn can lead to aberrant protein expression, and therapeutic agents which can target the alternative splicing events in genes can modulate the expression level of functional proteins in diseased patients and/or inhibit aberrant protein expression. Described herein are therapeutic agents and methods that can be used to treat a condition caused by these alternative splicing events.

### CROSS-REFERENCE

This application is a continuation of International Application No. PCT/US20/47081, filed Aug. 19, 2020 which claims the benefit of U.S. Provisional Application No. 62/888,887, filed Aug. 19, 2019, and U.S. Provisional Application No. 63/049,262, filed Jul. 8, 2020, each of which is incorporated herein by reference in its entirety.

### REFERENCE TO SEQUENCE LISTING

The instant application contains a “lengthy” Sequence Listing which has been submitted via CD-R in lieu of a printed paper copy, and is hereby incorporated by reference in its entirety. Said CD-R, recorded on Oct. 30, 2020, are labeled “CRF”, “Copy 1” and “Copy 2”, respectively, and each contains only one identical 107,372,032 bytes file (47991-727_601_SL.txt).

### BACKGROUND

Alternative splicing events in genes can lead to non-productive or less productive mRNA transcripts, and therapeutic agents which can target the alternative splicing events in genes can modulate the expression level of functional proteins in patients and/or inhibit aberrant protein expression. Such therapeutic agents can be used to treat a condition or disease caused by protein deficiency.

### SUMMARY

In some aspects, provided herein is a composition comprising a non-sense mediated RNA decay alternative exon (NSAE)-modulating agent that interacts with a target motif within a pre-processed mRNA transcript to modulate exclusion of an NSAE from a processed mRNA transcript and to modulate inclusion of a canonical exon in the processed mRNA transcript, wherein the target motif is located (i) in an intronic region between two canonical exons, (ii) in one of the two canonical exons, or (iii) in a region spanning both an intron and canonical exon; and wherein the NSAE comprises (i) only a portion of a canonical exon, or (ii) a canonical exon and at least a portion of an intron adjacent to the canonical exon; wherein the NSAE-modulating agent modulates exclusion of an NSAE from the processed mRNA transcript and modulates inclusion of a canonical exon in the processed mRNA transcript.

In some aspects, provided herein is a composition comprising a non-sense mediated RNA decay alternative exon (NSAE)-modulating agent that modulates expression of a target protein in a cell comprising a pre-processed mRNA transcript (pre-mRNA) that encodes the target protein and comprises: an alternative nonsense mediated RNA decay-inducing (NMD) exon comprising an alternative 5′ splice site downstream of the 5′ splice site of a canonical exon and within the canonical exon, or upstream of the 5′ splice site of the canonical exon and within an intron, wherein the NSAE-modulating agent modulates processing of an mRNA transcript from the pre-processed mRNA transcript by modulating splicing of the pre-mRNA at the 5′ alternative splice site, wherein the splicing of the pre-mRNA at the 5′ alternative splice site modulates the expression of the target protein in a cell.

In some aspects, provided herein is a composition comprising a non-sense mediated RNA decay alternative exon (NSAE)-modulating agent that modulates expression of a target protein in a cell comprising a pre-processed mRNA transcript (pre-mRNA) that encodes the target protein and comprises: an alternative nonsense mediated RNA decay-inducing (NMD) exon comprising an alternative 3′ splice site upstream of the 3′ splice site of a canonical exon and within the canonical exon, or downstream of the 3′ splice site of the canonical exon and within an intron, wherein the NSAE-modulating agent modulates processing of an mRNA transcript from the pre-processed mRNA transcript by modulating splicing of the pre-mRNA at the 3′ alternative splice site, and wherein the splicing of the pre-mRNA at the 3′ alternative splice site modulates the expression of the target protein in a cell.

In some aspects, provided herein is a composition comprising a non-sense mediated RNA decay alternative 5′ or 3′ splice site (NSASS)-modulating agent that interacts with a target motif within a pre-processed mRNA transcript to modulate splicing at an alternative 5′ or 3′ splice site of a pre-processed mRNA transcript and to modulate inclusion of a canonical exon in a processed mRNA transcript that is processed from the pre-processed mRNA transcript, wherein the target motif is located in an intronic region between two canonical exons, in one of the two canonical exons, or in a region spanning both an intron and canonical exon; wherein modulating splicing at the alternative 5′ or 3′ splice site of the pre-processed mRNA transcript modulates exclusion of an alternative exon from the processed mRNA transcript, wherein the alternative exon comprises only a portion of a canonical exon, or a canonical exon and at least a portion of an intron adjacent to the canonical exon; and wherein the NSASS-modulating agent modulates exclusion of the alternative exon from the processed mRNA transcript and modulates inclusion of a canonical exon in the processed mRNA transcript.

In some aspects, provided herein is a composition comprising a non-sense mediated RNA decay alternative 5′ or 3′ splice site (NSASS)-modulating agent that modulates expression of a target protein in a cell comprising a pre-processed mRNA transcript (pre-mRNA) that encodes the target protein, wherein the pre-mRNA comprises an alternative exon comprising an alternative 5′ splice site downstream of the 5′ splice site of a canonical exon and within the canonical exon, or upstream of the 5′ splice site of the canonical exon and within an intron; wherein the NSASS-modulating agent modulates processing of an mRNA transcript from the pre-processed mRNA transcript by modulating splicing of the pre-mRNA at the 5′ alternative splice site, wherein the splicing of the pre-mRNA at the 5′ alternative splice site modulates the expression of the target protein in a cell.

In some aspects, provided herein is a composition comprising a non-sense mediated RNA decay alternative exon (NSASS)-modulating agent that modulates expression of a target protein in a cell comprising a pre-processed mRNA transcript (pre-mRNA) that encodes the target protein, wherein the pre-mRNA comprises an alternative exon comprising an alternative 3′ splice site upstream of the 3′ splice site of a canonical exon and within the canonical exon, or downstream of the 3′ splice site of the canonical exon and within an intron, wherein the NSASS-modulating agent modulates processing of an mRNA transcript from the pre-processed mRNA transcript by modulating splicing of the pre-mRNA at the 3′ alternative splice site, and wherein the splicing of the pre-mRNA at the 3′ alternative splice site modulates the expression of the target protein in a cell.

In some embodiments, the agent is a small molecule. In some embodiments the agent is a polypeptide. In some embodiments, the polypeptide is a nucleic acid binding protein. In some embodiments, the nucleic acid binding protein contains a TAL-effector or zinc finger binding domain. In some embodiments, the nucleic acid binding protein is a Cas family protein. In some embodiments, the polypeptide is accompanied by or complexed with one or more nucleic acid molecules. In some embodiments, the Agent is an antisense oligomer (ASO) complementary to the targeted region of the pre-mRNA. In some embodiments, the Agent is at least 75%, at least 80%, at least 85%, at least 90%, at least 95%, at least 98%, at least 99%, or 100%, complementary to the targeted region of the pre-mRNA encoding the target protein. In some embodiments, the Agent is an antisense oligomer, and wherein the Agent comprises a backbone modification comprising a phosphorothioate linkage or a phosphorodiamidate linkage. In some embodiments, the Agent is an antisense oligomer, and wherein the Agent comprises a phosphorodiamidate morpholino. In some embodiments, the Agent is an antisense oligomer, and wherein the Agent comprises a locked nucleic acid. In some embodiments, the Agent is an antisense oligomer, and wherein the Agent comprises a peptide nucleic acid. In some embodiments, the Agent is an antisense oligomer, and wherein the Agent comprises a 2′-O-methyl. In some embodiments, the Agent is an antisense oligomer, and wherein the Agent comprises a 2′-Fluoro, or a 2′-O-methoxyethyl moiety. In some embodiments, the Agent is an antisense oligomer, and wherein the Agent comprises at least one modified sugar moiety. In some embodiments, each sugar moiety is a modified sugar moiety. In some embodiments, the Agent is an antisense oligomer, and wherein the Agent consists of from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, or 12 to 15 nucleobases. Described herein, in some aspects, is a composition of a nucleic acid molecule that encodes for the NSAE-modulating agent. In some embodiments, the nucleic acid molecule is incorporated into a viral delivery system. In some embodiments, the viral delivery system is an adenovirus-associated vector.

In some aspects, provided herein is a method of modulating protein expression, comprising: (a) contacting a non-sense mediated RNA decay alternative exon (NSAE)-modulating agent to a target motif within a pre-processed mRNA transcript, wherein the NSAE comprises (i) only a portion of a canonical exon, or (ii) a canonical exon and at least a portion of an intron adjacent to the canonical exon; (b) processing a mRNA transcript from the pre-processed mRNA transcript thereby forming a processed mRNA transcript, wherein the NSAE-modulating agent modulates exclusion of an NSAE from the processed mRNA transcript and modulates inclusion of the canonical exon in the processed mRNA transcript; and (c) translating the processed mRNA transcript wherein the exclusion of the NSAE and inclusion of the canonical exon modulates protein expression relative to the protein expression of an equivalent mRNA transcript comprising the NSAE instead of the canonical exon. In some embodiments, the target motif is located in an intronic region between two canonical exons. In some embodiments, the target motif is located in one of the two canonical exons. In some embodiments, the target motif is located in a region spanning both an intron and a canonical exon.

In some aspects, provided herein is a method of modulating expression of a target protein by a cell having a pre-processed mRNA transcript (pre-mRNA) that encodes the target protein and comprises: an alternative nonsense mediated RNA decay-inducing (NMD) exon comprising an alternative 3′ splice site upstream of the 3′ splice site of a canonical exon and within the canonical exon, or downstream of the 3′ splice site of the canonical exon and within an intron, the method comprising contacting a non-sense mediated RNA decay alternative exon (NSAE)-modulating agent to the cell, wherein the non-sense mediated RNA decay alternative exon (NSAE)-modulating agent modulates processing of an mRNA transcript from the pre-processed mRNA transcript by modulating splicing of the pre-mRNA at the 3′ alternative splice site, and wherein the splicing of the pre-mRNA at the 3′ alternative splice site modulates the expression of the target protein.

In some aspects, provided herein is a method of modulating expression of a target protein by a cell having a pre-processed mRNA transcript (pre-mRNA) that encodes the target protein and comprises: an alternative nonsense mediated RNA decay-inducing (NMD) exon comprising an alternative 5′ splice site downstream of the 5′ splice site of a canonical exon and within the canonical exon, or upstream of the 5′ splice site of the canonical exon and within an intron, the method comprising contacting a non-sense mediated RNA decay alternative exon (NSAE)-modulating agent to the cell, wherein the non-sense mediated RNA decay alternative exon (NSAE)-modulating agent modulates processing of an mRNA transcript from the pre-processed mRNA transcript by modulating splicing of the pre-mRNA at the 5′ alternative splice site, and wherein the splicing of the pre-mRNA at the 5′ alternative splice site modulates the expression of the target protein.

In some embodiments, the non-sense mediated RNA decay alternative exon (NSAE)-modulating agent binds to a targeted portion of the pre-processed mRNA transcript. In some embodiments, the wherein the non-sense mediated RNA decay alternative exon (NSAE)-modulating agent binds to a factor involved in splicing of the NSAE or NMD exon. In some embodiments, the wherein the non-sense mediated RNA decay alternative exon (NSAE)-modulating agent inhibits activity of a factor involved in splicing of the NMD exon. In some embodiments, the wherein the non-sense mediated RNA decay alternative exon (NSAE)-modulating agent interferes with binding of a factor involved in splicing of the NMD exon to a region of the targeted portion of the pre-processed mRNA transcript. In some embodiments, modulation of splicing of the pre-mRNA increases the expression of the target protein. In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, modulation of splicing of the pre-mRNA increases production of the processed mRNA encoding the target protein. In some embodiments, the level of processed mRNA encoding the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the target protein is the canonical isoform of the protein. In some embodiments, the target protein is SynGAP1. In some embodiments, the target protein is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the non-sense mediated RNA decay alternative exon (NSAE)-modulating agent is a composition described herein.

In some aspects, provided herein is a pharmaceutical composition comprising: a therapeutic agent comprising a composition described herein; and a pharmaceutically acceptable excipient and/or a delivery vehicle.

In some aspects, provided herein is a method of treating or preventing a disease or condition in a subject in need thereof, the method comprising: administering to the subject a pharmaceutical composition described herein.

In some aspects, provided herein is a method of treating or preventing a disease or condition in a subject in need thereof, the method comprising: administering to the subject a pharmaceutical composition comprising: (a) a non-sense mediated RNA decay alternative exon (NSAE)-modulating agent that interacts with a target motif within a pre-processed mRNA transcript to modulate exclusion of an NSAE from a processed mRNA transcript and to modulate inclusion of a canonical exon in the processed mRNA transcript, wherein the NSAE comprises (i) only a portion of a canonical exon, or (ii) a canonical exon and at least a portion of an intron adjacent to the canonical exon; and (b) a pharmaceutically acceptable excipient and/or a delivery vehicle, wherein the disease or condition is treated or prevented in the subject by the administration of the NSAE-modulating agent by a modulation in expression of a protein translated from the processed mRNA transcript.

In some aspects, provided herein is a method of treating a disease or condition in a subject in need thereof by modulating expression of a target protein in a cell of the subject, wherein the cell of the subject has a pre-processed mRNA transcript (pre-mRNA) that encodes the target protein and comprises: (a) an exon followed by an intron flanking a 3′ splice site of the exon; and (b) an alternative nonsense mediated RNA decay-inducing (NMD) exon comprising an alternative 3′ splice site upstream of the 3′ splice site of the exon and within the exon, or downstream of the 3′ splice site of the exon and within the intron, the method comprising contacting a therapeutic agent to the cell, wherein the therapeutic agent modulates processing of an mRNA transcript from the pre-processed mRNA transcript by modulating splicing of the pre-mRNA at the 3′ alternative splice site, and wherein the splicing of the pre-mRNA at the 3′ alternative splice site modulates the expression of the target protein in the cell of the subject.

In some aspects, provided herein is a method of treating a disease or condition in a subject in need thereof by modulating expression of a target protein in a cell of the subject, wherein the cell of the subject has a pre-processed mRNA transcript (pre-mRNA) that encodes the target protein and comprises: (a) an exon preceded by an intron flanking a 5′ splice site of the exon; and (b) an alternative nonsense mediated RNA decay-inducing (NMD) exon comprising an alternative 5′ splice site downstream of the 5′ splice site of the exon and within the exon, or upstream of the 5′ splice site of the exon and within the intron, the method comprising contacting a therapeutic agent to the cell, wherein the therapeutic agent modulates processing of an mRNA transcript from the pre-processed mRNA transcript by modulating splicing of the pre-mRNA at the 5′ alternative splice site, and wherein the splicing of the pre-mRNA at the 5′ alternative splice site modulates the expression of the target protein in the cell of the subject.

In some embodiments the disease is autosomal dominant mental retardation, epileptic encephalopathy, or autism. In some embodiments, the disease or the condition is caused by a deficient amount or activity of the target protein. In some embodiments, the therapeutic agent increases the level of the processed mRNA encoding the target protein in the cell. In some embodiments, the therapeutic agent increases the expression of the target protein in the cell. In some embodiments, the level of processed mRNA encoding the target protein in the cell contacted with the therapeutic agent is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the level the target protein in the cell is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the method further comprises assessing mRNA levels or expression levels of the target protein. In some embodiments, the method further comprises assessing the subject's genome for at least one genetic mutation associated with the disease.

In some embodiments, the disease or the condition is caused by an excess amount or activity of the target protein. In some embodiments, the therapeutic agent decreases the level of the processed mRNA encoding the target protein in the cell. In some embodiments, the therapeutic agent decreases the expression of the target protein in the cell. In some embodiments, the level of processed mRNA encoding the target protein in the cell contacted with the therapeutic agent is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell. In some embodiments, the level the target protein in the cell is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the level of processed mRNA encoding the target protein in a control cell.

In some embodiments, at least one genetic mutation is within a locus of a gene associated with the disease. In some embodiments, at least one genetic mutation is within a locus associated with expression of a gene associated with the disease. In some embodiments, at least one genetic mutation is within the SYNGAP1 gene locus. In some embodiments, at least one genetic mutation is within a locus associated with SYNGAP1 gene expression. In some embodiments, at least one genetic mutation is within a target gene locus, wherein the target gene locus is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 gene locus. In some embodiments, at least one genetic mutation is within a locus associated with a target gene expression, wherein the target gene is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 genes. In some embodiments, the subject is a human. In some embodiments, the subject is a non-human animal. In some embodiments, the subject is a fetus, an embryo, or a child. In some embodiments, the cell or the cells is ex vivo, or in a tissue, or organ ex vivo. In some embodiments, the therapeutic agent is administered to the subject by intracerebroventricular injection, intraperitoneal injection, intramuscular injection, intrathecal injection, subcutaneous injection, oral administration, synovial injection, intravitreal administration, subretinal injection, topical application, implantation, or intravenous injection.

In some aspects, provided herein is a therapeutic agent for use in a method described herein.

In some aspects, provided herein is a pharmaceutical composition comprising a therapeutic agent described herein and a pharmaceutically acceptable excipient.

In some aspects, provided herein is a method of treating a subject in need thereof, comprising administering a pharmaceutical composition described herein by intracerebroventricular injection, intraperitoneal injection, intramuscular injection, intrathecal injection, subcutaneous injection, oral administration, synovial injection, intravitreal administration, subretinal injection, topical application, implantation, or intravenous injection to the subject.

### BRIEF DESCRIPTION OF THE DRAWINGS

FIG. 1A depicts an exemplary schematic depicting a wild type cell expressing a pre-mRNA that is alternatively spliced to generate a productive mRNA and a non-productive mRNA. While the non-productive mRNA is degraded by NMD, the productive mRNA is translated into normal protein levels.

FIG. 1B depicts an exemplary schematic depicting a wild type cell treated with a TANGO (Targeted Augmentation of Nuclear Gene Output) ASO targeting the non-productive AS event (e.g. exon inclusion) that prevents the inclusion of the non-productive exon and leads to an increase of the productive mRNA. Increased level of the productive mRNA results in increased protein level beyond 100%.

FIG. 1C depicts an exemplary schematic depicting an example of a haploinsufficient cell containing a wild-type allele (orange) and a mutant allele (red). Both alleles express a pre-mRNA that is alternatively spliced to generate a productive mRNA and a non-productive mRNA. While the non-productive mRNAs are degraded by NMD, the productive mRNA from the wild-type allele generates 50% of normal protein levels leading to disease.

FIG. 1D depicts an exemplary schematic depicting an example of a haploinsufficient cell treated with a TANGO ASO targeting the non-productive AS event (e.g. exon inclusion) that prevents the inclusion of the non-productive exon and leads to an increase of the productive mRNA from both alleles. While the mutant mRNA won't produce a functional protein, increased levels of the wild type productive mRNA restores the protein level back to normal.

FIG. 1E depicts an exemplary schematic depicting an alternative 5′ ss of an exon (Exon B) (which corresponds to an alternative 3′ ss of the intron preceding the exon (Intron A). In this example, the alternative exon is longer than the corresponding canonical exon and the alternative intron is shorter than the corresponding canonical intron.

FIG. 1F depicts an exemplary schematic depicting an alternative 5′ ss of an exon (Exon B) (which corresponds to an alternative 3′ ss of the intron preceding the exon (Intron A). In this example, the alternative exon is shorter than the corresponding canonical exon and the alternative intron is longer than the corresponding canonical intron.

FIG. 1G depicts an exemplary schematic depicting an alternative 3′ ss of an exon (Exon B) (which corresponds to an alternative 5′ ss of the intron following the exon (Intron B). In this example, the alternative exon is longer than the corresponding canonical exon and the alternative intron is shorter than the corresponding canonical intron.

FIG. 1H depicts an exemplary schematic depicting an alternative 3′ ss of an exon (Exon B) (which corresponds to an alternative 5′ ss of the intron following the exon (Intron B). In this example, the alternative exon is shorter than the corresponding canonical exon and the alternative intron is longer than the corresponding canonical intron.

FIG. 2 depicts examples of non-productive alternative splicing events that are predicted to lead to premature termination codons and undergo non-sense mediated decay of processed mRNA transcripts. The left panel depicts the types of alternative splicing events (cassette exons, alternative splice sites, and alternative intron) that are predicted to lead to the introduction of a premature termination codon (PTC) and transcript degradation. The horizontal bar graph summarizes the number of protein-coding genes containing each of the non-productive alternative splicing event type. Non-disease-associated genes are depicted by the top bars and disease-associated genes are depicted by bottom bars (based on Orphanet disease database). Exons are denoted by rectangles and introns by lines. Dark gray rectangles or lines denote a non-productive alternative splicing region.

FIG. 3 depicts an exemplary schematic representation of SYNGAP1 NMD-inducing alternative 3′ splice site of an intron (which corresponds to an alternative 5′ ss of the exon downstream of the alternative intron). NMD-inducing alt 3′ss event: UCSC Genome Browser snapshot of a region in the SYNGAP1 gene (exons are rectangles and introns are lines with arrowheads) that contains an NMD-inducing Alternative 3′ splice site event (chr6 3344055333440728) depicted by the shaded area and black bar on the top. RNA sequencing traces from human middle frontal gyrus samples from individuals at various ages and proliferating or differentiated RenCellVM cells treated with cycloheximide (CHX) or DMSO control are shown.

FIG. 4 depicts an exemplary ASO walk design. The shaded nucleotides correspond to the portion of the extended exon 11 of SYNGAP1 that results from the selection of the indicated Alt 3′ss of the intron (which corresponds to the alternative 5′ ss of the exon downstream of the alternative intron). Figure discloses SEQ ID NOs: 10226-10228, respectively, in order of appearance.

FIGS. 5A-5C depict graphs showing exemplary ASO walk Taqman qPCR results, e.g., ASO screening by Tagman qPCR.

FIG. 5A depicts exemplary Taqman qPCR results using RNA from HEK293 cells transfected for 24 hrs with 80 nM ASOs from region 1 depicted in FIG. 4. The exemplary Taqman probe spans exons 10 and 11 junction and measures productive mRNA.

FIG. 5B depicts exemplary Taqman qPCR results using RNA from HEK293 cells transfected for 24 hrs with 80 nM ASOs from region 2 depicted in FIG. 4. The exemplary Taqman probe spans exons 10 and 11 junction and measures productive mRNA.

FIG. 5C depicts exemplary Taqman qPCR results using RNA from HEK293 cells transfected for 24 hrs with 80 nM ASOs from region 3 depicted in FIG. 4. The exemplary Taqman probe spans exons 10 and 11 junction and measures productive mRNA.

FIG. 6 depicts the use of antisense oligomers to modulate non-productive alternative splicing events and increase mRNA and protein levels in a dose-dependent manner in vitro. A selected SYNGAP1 ASO targeting an alternative 3′ splice site event was transfected in HEK293 (human embryonic kidney cell) cells at increasing concentrations for 24h and RT-PCR (reverse transcription polymerase chain reaction) and TaqMan qPCR (quantitative polymerase chain reaction) was performed. The bar graph on the left shows a dose-dependent reduction of the non-productive mRNA and the bar graph on the right shows a dose-dependent increase of productive mRNA. A Western blot and protein quantification for SynGAP from HEK293 cells treated with the respective selected ASOs at increasing concentrations for 48 hrs is shown on the right. Equal protein loading was confirmed with Ponceau staining. Fc=fold change over control.

FIG. 7 depicts exemplary RT-PCR and densitometric quantifications of RT-PCR validation in cells treated with 50 pg/mL of cycloheximide (CHX) or equal volume of DMSO (Dimethyl sulfoxide) for 3 hours for SYNGAP1.

FIG. 8 depicts the dose-dependent effect of antisense oligomers that modulate non-productive alternative splicing events. A representative RT-PCR PAGE (polyacrylamide gel electrophoresis) of cells transfected with increasing concentrations of selected antisense oligomers targeting an alternative 3′ss in SYNGAP1 is depicted.

FIG. 9 depicts an exemplary Western blot of SynGAP following treatment with 100 nM siRNA against SYNGAP1 for 48 hours in HEK293 cells.

FIGS. 10A-10D depict an exemplary schematic representation, pictures and a graph showing validation of alternative 3′ splice site event of an intron (intron 10) (which corresponds to an alternative 5′ ss of the exon downstream of the alternative intron (exon 11)). *Mouse event has a different size than human event.

FIG. 10A depicts an exemplary schematic representation of an alternative 3′ splice site (Alt 3′ss) event of an intron in SYNGAP1 (intron 10) (which corresponds to an alternative 5′ ss of the exon downstream of the alternative intron (exon 11)).

FIG. 10B depicts exemplary results of RT-PCR to SYNGAP1 using RNA from RenCellsVM (Neural progenitor cells) or Neuro 2A (mouse neuroblastoma cells) treated with either DMSO (−) or cycloheximide (CHX) (+). Primers were positioned in exon 10 and 11.

FIG. 10C depicts exemplary results of RT-PCR to SYNGAP1 using RNA from total mouse brain. Primers were positioned in exon 10 and 11.

FIG. 10D depicts an exemplary quantification of the RT-PCR SYNGAP1 products using RNA from various non-human primates brain regions plotted as percentage of Alt intron 3′ss isoform (Alt downstream exon 5′ss isoform) (Alt intron 3′ss isoform/(Alt intron 3′ss isoform+productive mRNA)*100, i.e. Alt downstream exon 5′ss isoform/(Alt downstream exon 5′ss isoform+productive mRNA).

FIGS. 11A-11C depict graphs and pictures showing that an exemplary ASO prevents Alt 3′ss selection and increases SYNGAP1 mRNA and protein.

FIG. 11A and FIG. 11B depict exemplary RT-PCR results showing reduction of SYNGAP1 Alt 3′ss isoform and exemplary Tagman qPCR results showing increase of productive mRNA, respectively.

FIG. 11A shows quantification of the RT-PCR products using RNA from HEK293 cells transfected for 24 hrs with selected ASO #1 from region 2 plotted as percentage of Alt intron 3′ss isoform (Alt downstream exon 5′ss isoform) (Alt intron 3′ss isoform/(Alt intron 3′ss isoform+productive mRNA)*100, i.e. Alt downstream exon 5′ss isoform/(Alt downstream exon 5′ss isoform+productive mRNA). Primers were positioned in exons 10 and 11. N=2. NT=non-targeting ASO control.

FIG. 11B shows Taqman qPCR results using a probe that spans the exon 10-11 junction that measures productive mRNA plotted as fold change over no ASO control (−) using the same samples as in FIG. 11A. N=2. NT=non-targeting ASO control.

FIG. 11C depicts exemplary Western blot results showing increase of SynGAP protein. FIG. 11C shows Western blot analysis using whole cell lysate from HEK293 cells transfected for 48 hrs with a selected ASO. Quantification of bands corresponding to SynGAP were normalized to Ponceau loading control and fold change (fc) over control (−) is shown under the gel.

FIGS. 12A-12E depict pictures and graphs showing that exemplary selected MOE (2-methoxyethyl)) lengthmers and PMOs (phosphorodiamidate morpholinos) prevent Alt intron 3′ss selection (Alt downstream exon 5′ss selection) and increase SYNGAP1 mRNA and protein.

FIG. 12A and FIG. 12B depict exemplary RT-PCR results showing reduction of SYNGAP1 Alt intron 3′ss isoform (Alt downstream exon 5′ss isoform).

FIG. 12A shows RT-PCR results using RNA from MEF (mouse embryonic fibroblasts) nucleofected for 24 hrs with selected MOE #5 from region 1 and derivative MOE lengthmers and PMOs. Primers were positioned in exons 10 and 11.

FIG. 12B shows quantifications of RT-PCR products shown in FIG. 12A plotted as percentage of Alt intron 3′ss isoform (Alt downstream exon 5′ss isoform) (Alt intron 3′ss isoform/(Alt intron 3′ss isoform+productive mRNA)*100, i.e. Alt downstream exon 5′ss isoform/(Alt downstream exon 5′ss isoform+productive mRNA). N=2. −=no ASO control.

FIG. 12C depicts exemplary Tagman qPCR results showing increase of productive mRNA. FIG. 12C shows Taqman qPCR results using a probe that spans the exon 10-11 junction that measures productive mRNA plotted as fold change over no ASO control (−) using the same samples as in FIG. 12A. N=2. −=no ASO control.

FIG. 12D and FIG. 12E depict exemplary Western blot results showing increase of SynGAP protein.

FIG. 12D shows Western blot analysis using whole cell lysate from MEFs nucleofected for 48 hrs with selected ASO #5 (MOE) and #29 (PMO) I.

FIG. 12E shows quantification of bands corresponding to SynGAP normalized to Ponceau loading control and plotted as fold change (fc) over control (−). N=2. −=no ASO control.

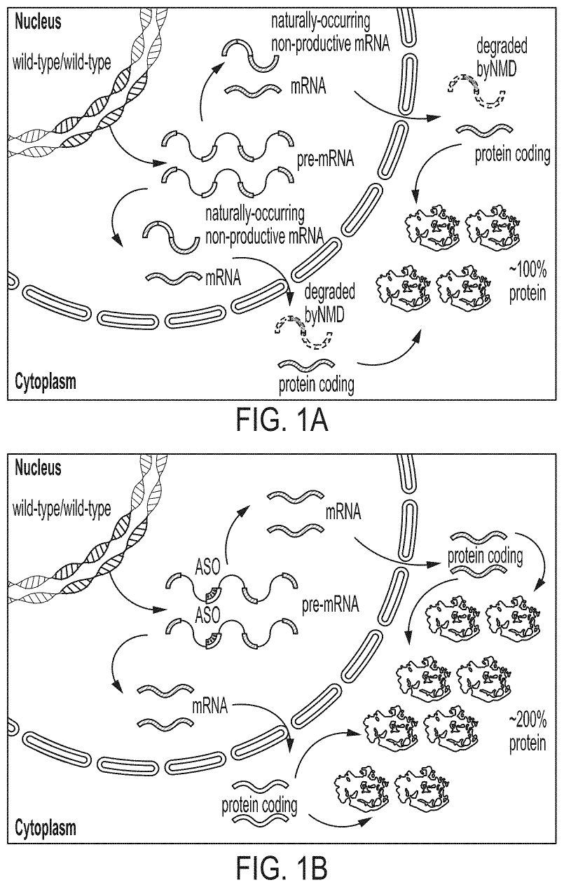

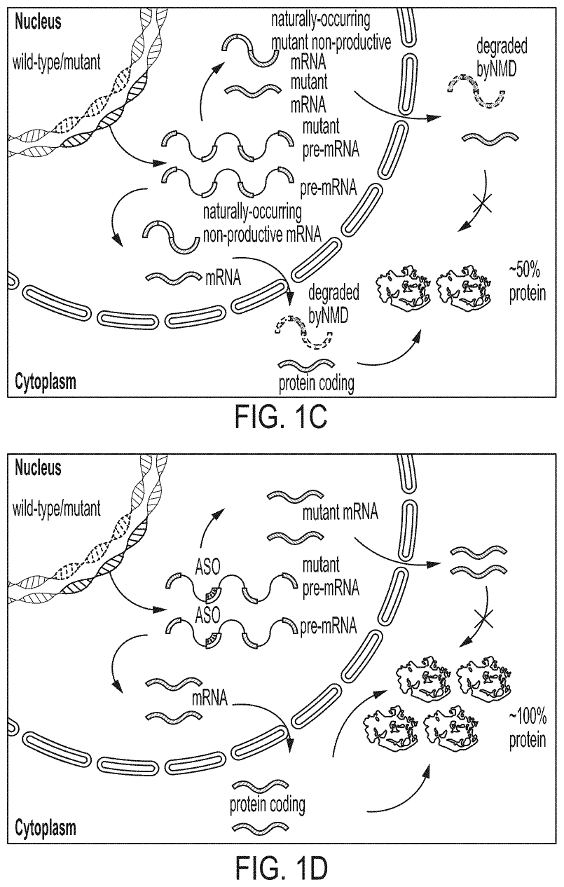

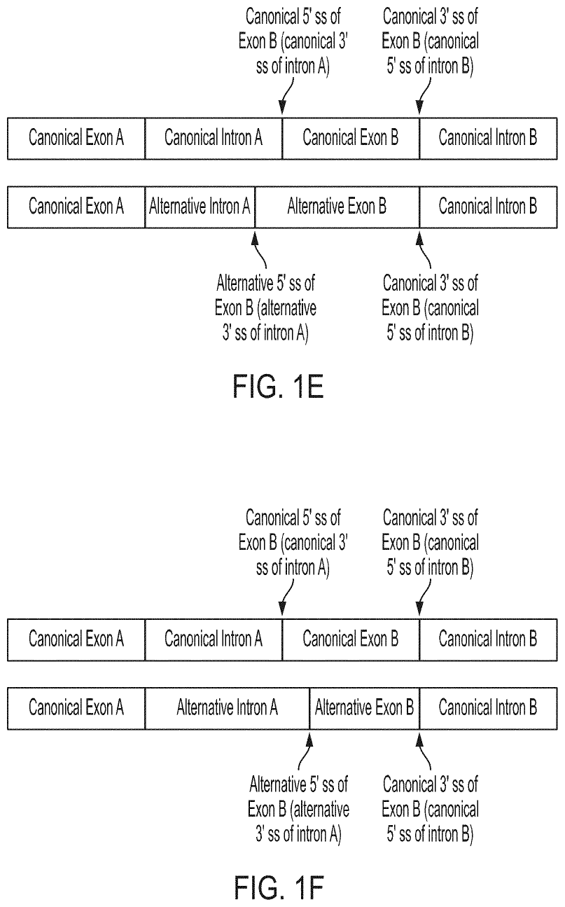

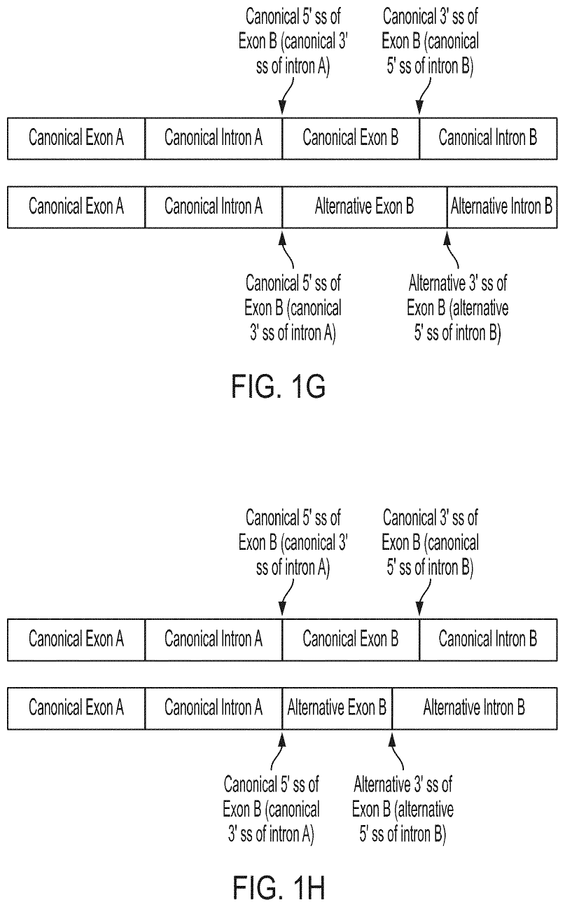

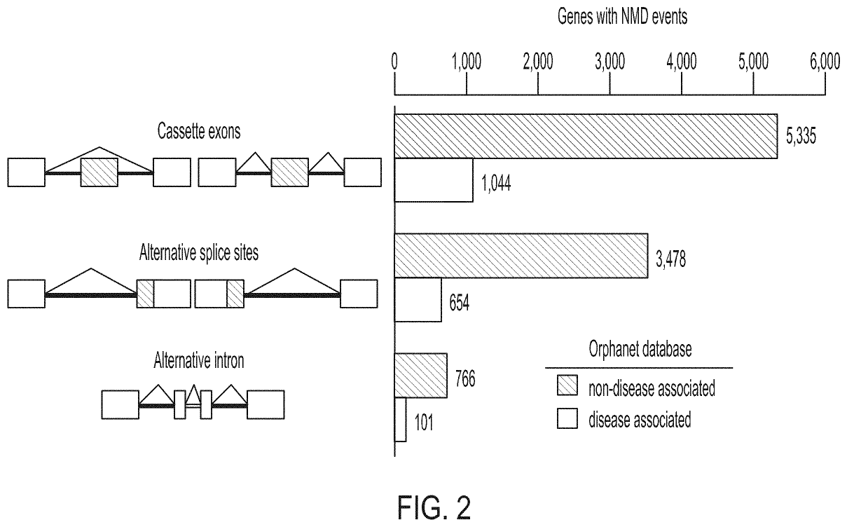

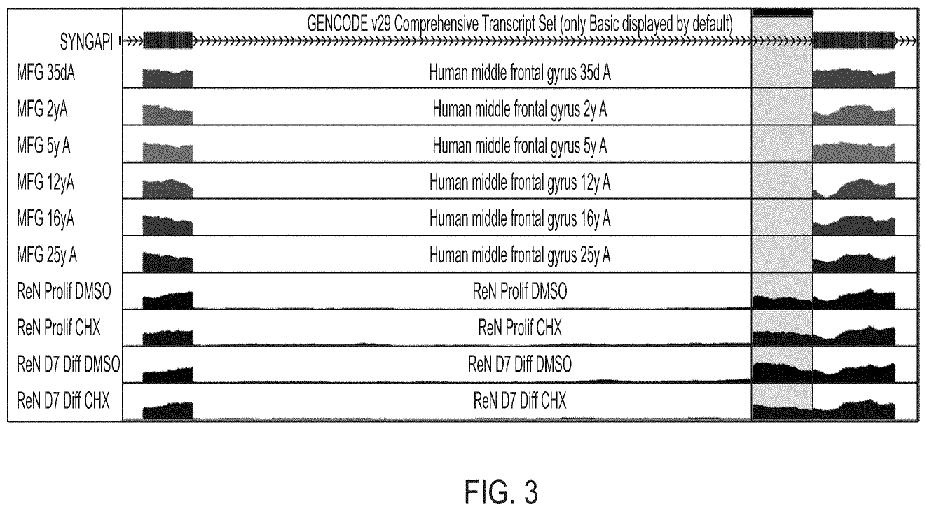

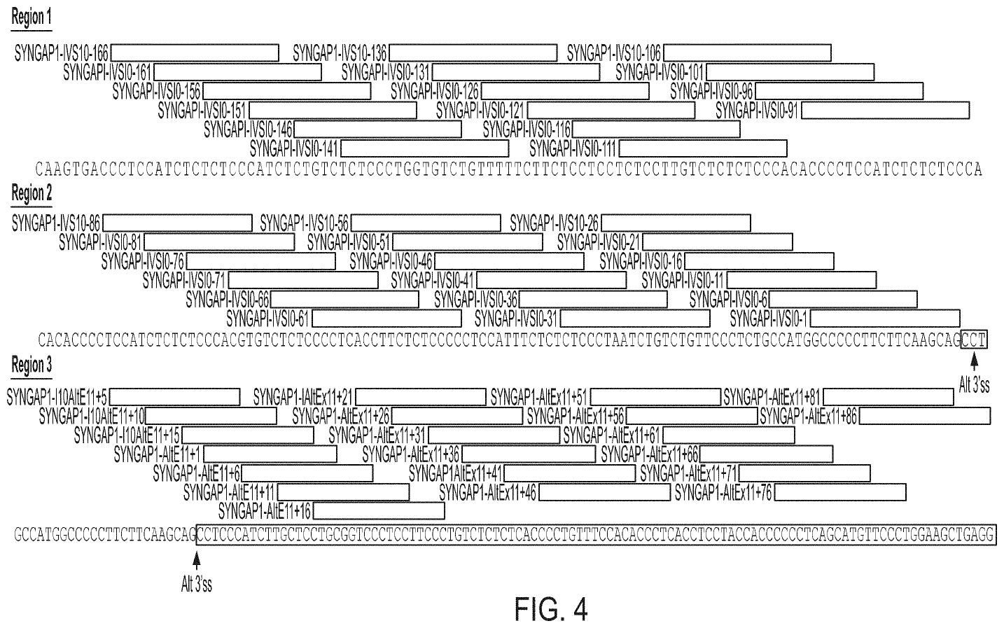

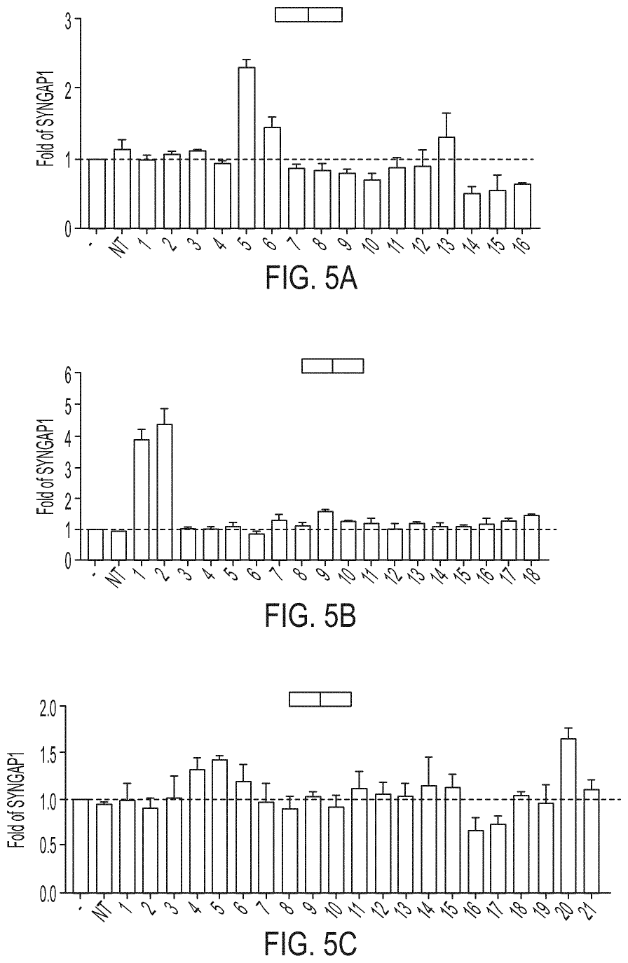

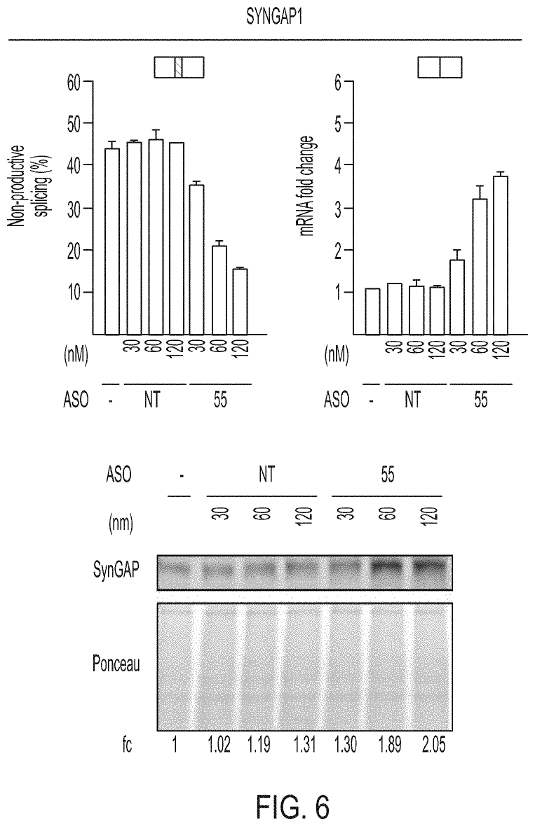

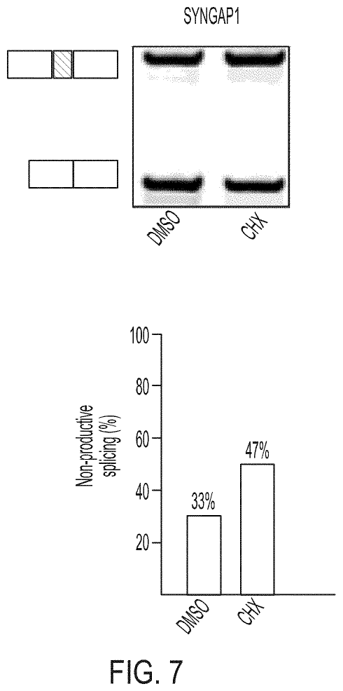

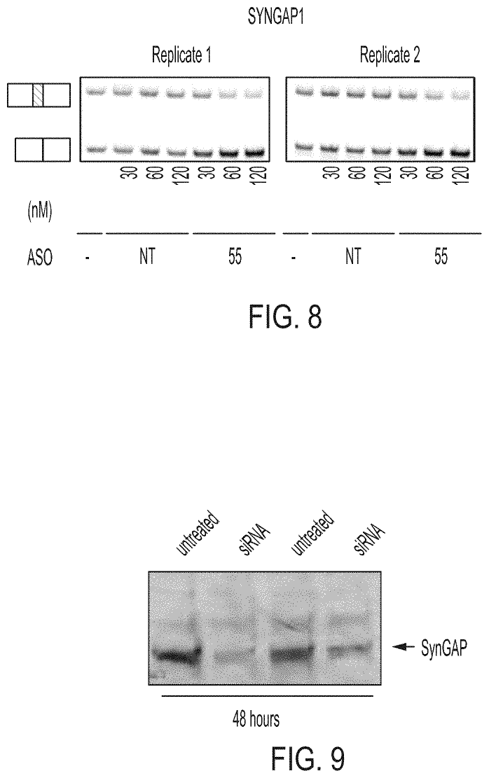

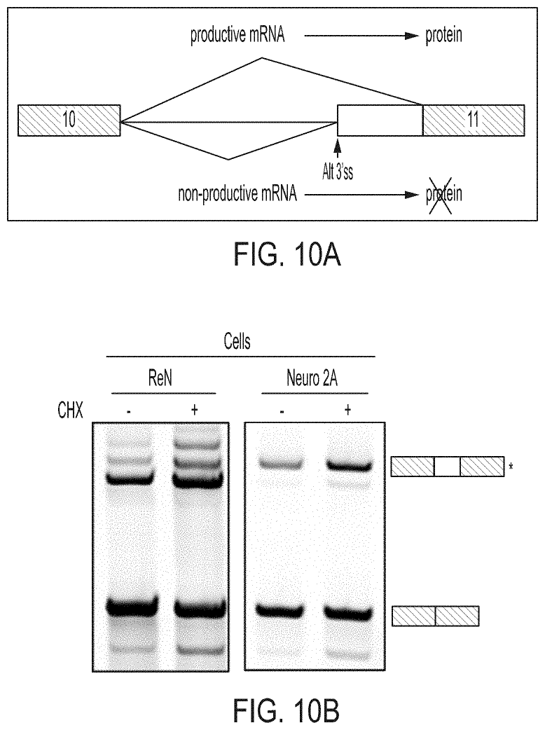

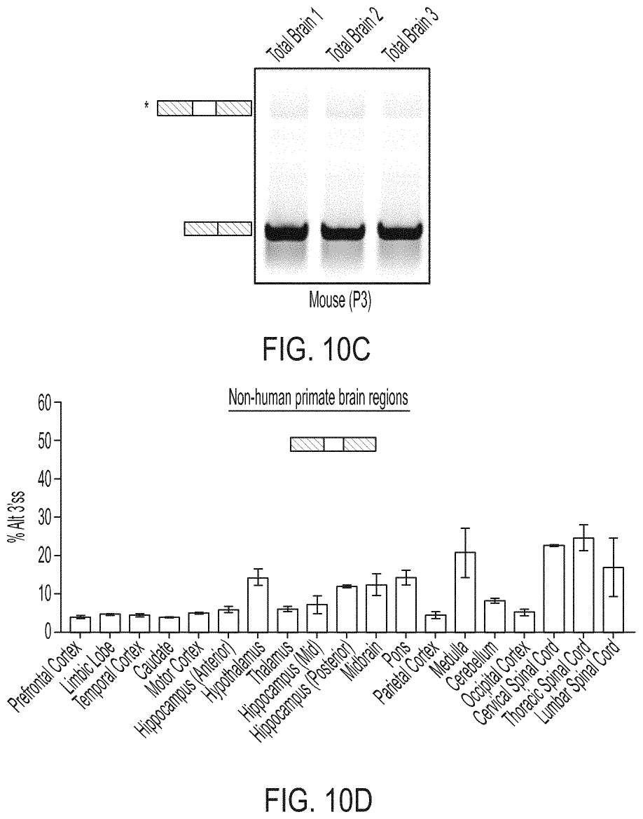

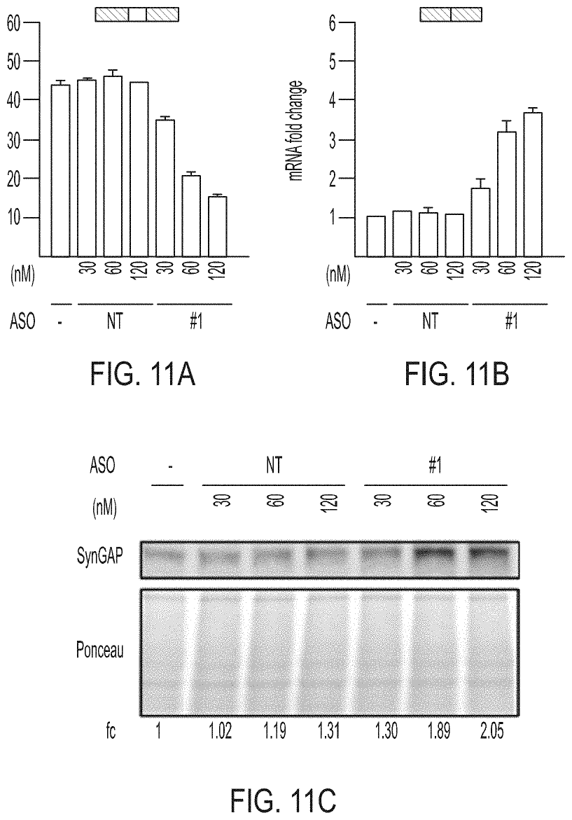

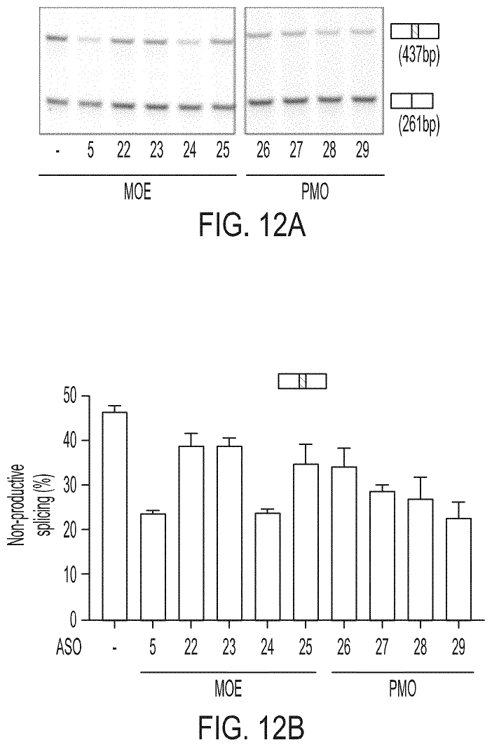

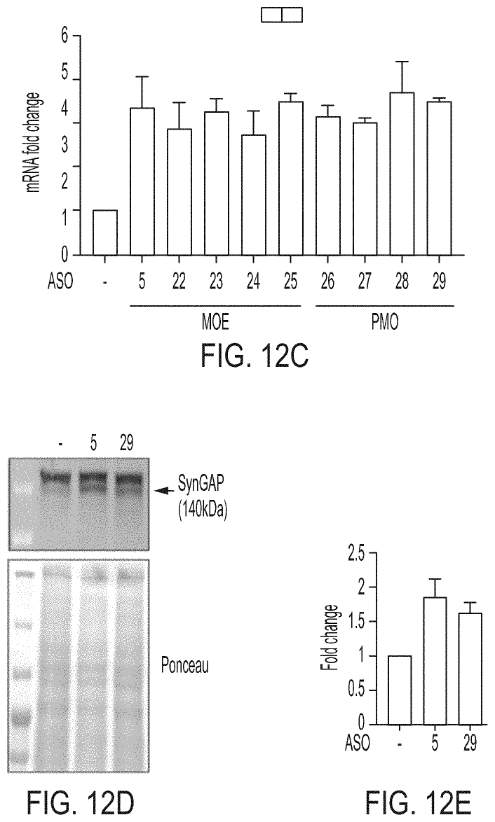

### DETAILED DESCRIPTION

Certain specific details of this description are set forth in order to provide a thorough understanding of various embodiments. However, one skilled in the art will understand that the present disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The coordinate as used herein refers to the coordinate of the genome reference assembly GRCh38 (Genome Research Consortium human build 38), also known as Hg38 (Human genome build 38).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below.

As used herein, an alternative 3′ splice site of an intron is equivalent to an alternative 5′ splice site of the exon immediately downstream of that intron.

As used herein, an alternative 5′ splice site of an intron is equivalent to an alternative 3′ splice site of the exon immediately upstream of that intron.

### Autosomal Dominant Mental Retardation and SYNGAP1 Gene

Autosomal dominant mental retardation is an autosomal dominant neurodevelopmental disorder characterized by significantly impaired intellectual functioning and adaptive behaviors, one cause of which is haploinsufficency of SYNGAP1 gene expression. SYNGAP1 codes for SYNGAP1 protein, which is a ras GTPase-activating protein involved in cognitive development and proper synapse function amongst neurons. SYNGAP1 is known to be involved in synaptic plasticity, axon formation, and AMPA receptor trafficking. Generally, SYNGAP1 activity is essential for development and survival. SYNGAP1 deficiencies may be caused by sporadic mutation or alternative splicing events. Deficient SYNGAP1 activity may result in mental retardation, or intellectual disability, epileptic encephalopathy, or autism.

The present disclosure provides compositions and methods for modulating alternative splicing of SYNGAP1 to modulate the production of functional protein-coding mature mRNA, and thus, translated functional SYNGAP1 protein. These compositions and methods include antisense oligomers (ASOs) that can promote canonical splicing of SYNGAP1 pre-mRNA. In various embodiments, functional SYNGAP1 protein can be increased using the methods of the disclosure to treat a condition caused by SYNGAP1 protein deficiency. In some embodiments, the condition is autosomal dominant mental retardation. In other embodiments, the condition is epileptic encephalopathy or autism.

In some embodiments, the methods of the invention are used to increase functional SYNGAP1 protein production to treat a condition in a subject in need thereof. In some embodiments, the subject has a condition in which SYNGAP1 is not necessarily deficient relative to wild-type, but where an increase in SYNGAP1 mitigates the condition nonetheless. In some embodiments, the condition is caused by sporadic mutation. In some embodiments, the methods of the invention are used to reduce functional SYNGAP1 protein production to treat a condition in a subject in need thereof. In some embodiments, the methods of the invention are used to modulate functional SYNGAP1 protein production to treat a condition in a subject in need thereof.

### Target Genes

The present disclosure provides compositions and methods for modulating alternative splicing of a target to modulate the production of functional protein-coding mature mRNA, and thus, translated functional the target protein, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. These compositions and methods include antisense oligomers (ASOs) that can promote canonical splicing of the target pre-mRNA, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In various embodiments, functional target protein can be increased using the methods of the disclosure to treat a condition caused by target protein deficiency, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the condition is autosomal dominant mental retardation. In other embodiments, the condition is epileptic encephalopathy or autism.

In some embodiments, the methods of the invention are used to increase functional the target protein production to treat a condition in a subject in need thereof, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the subject has a condition in which the target protein is not necessarily deficient relative to wild-type, but where an increase in the target protein mitigates the condition nonetheless, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the condition is caused by sporadic mutation. In some embodiments, the methods of the invention are used to reduce functional target protein production to treat a condition in a subject in need thereof, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the methods of the invention are used to modulate functional target protein production to treat a condition in a subject in need thereof, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14.

### Splicing

Intervening sequences or introns are removed by a large and highly dynamic RNA-protein complex termed the spliceosome, which orchestrates complex interactions between primary transcripts, small nuclear RNAs (snRNAs) and a large number of proteins. Spliceosomes assemble ad hoc on each intron in an ordered manner, starting with recognition of the 5′ splice site (5′ss) by U1 snRNA or the 3′splice site (3′ss), which involves binding of the U2 auxiliary factor (U2AF) to the 3′ss region to facilitate U2 snRNA binding to the branch point sequence (BPS). U2AF is a stable heterodimer composed of a U2AF2-encoded 65-kD subunit (U2AF65), which binds the polypyrimidine tract (PPT), and a U2AF1-encoded 35-kD subunit (U2AF35), which interacts with highly conserved AG dinucleotides at 3′ss and stabilizes U2AF65 binding. In addition to the BPS/PPT unit and 3′ss/5′ss, accurate splicing requires auxiliary sequences or structures that activate or repress splice site recognition, known as intronic or exonic splicing enhancers or silencers. These elements allow genuine splice sites to be recognized among a vast excess of cryptic or pseudo-sites in the genome of higher eukaryotes, which have the same sequences but outnumber authentic sites by an order of magnitude. Although they often have a regulatory function, the exact mechanisms of their activation or repression are poorly understood.

The decision as to whether splicing occurs can be typically modeled as a stochastic rather than deterministic process, such that even the most defined splicing signals can sometimes splice incorrectly. However, under normal conditions, pre-mRNA splicing proceeds with surprisingly high accuracy. This may be attributed in part to the activity of adjacent cis-acting auxiliary exonic and intronic splicing regulatory elements (ESRs or ISRs). Typically, these functional elements are classified as either exonic or intronic splicing enhancers (ESEs or ISEs) or silencers (ESSs or ISSs) based on their ability to stimulate or inhibit splicing, respectively. Although there is now evidence to suggest that some auxiliary cis-acting elements may act by influencing the kinetics of spliceosome assembly, such as the arrangement of the complex between U1 snRNP and the 5′ss, it seems highly likely that multiple elements function in a coordinated manner with trans-acting RNA-binding proteins (RBPs). For example, the serine- and arginine-rich family of RBPs (SR proteins) is a conserved family of proteins that play a key role in defining exons. SR proteins promote exon recognition by recruiting components of the pre-spliceosome to adjacent splice sites or by antagonizing the effects of ESSs in the vicinity. The repressive effects of ESSs and ISSs can be mediated by members of the heterogeneous nuclear ribonucleoprotein (hnRNP) family and can alter recruitment of core splicing factors to adjacent splice sites. In addition to their roles in splicing regulation, silencer elements have been suggested to play a role in the repression of pseudo-exons, which are sets of decoy intronic splice sites with the typical spacing of an exon but without a functional open reading frame. ISEs, ISSs, ESEs and ESSs, in cooperation with their related trans-acting RBPs, represent important components in a set of splicing controls that specify how, where and when mRNAs are assembled from their precursors.

The sequences marking the exon-intron boundaries are degenerate signals of varying strengths that can occur at high frequency within human genes. In multi-exon genes, different pairs of splice sites can be linked together in many different combinations, creating a diverse array of transcripts from a single gene. This is commonly referred to as alternative pre-mRNA splicing. Although most mRNA isoforms produced by alternative splicing can be exported from the nucleus and translated into functional polypeptides, different mRNA isoforms from a single gene can vary greatly in their translation efficiency. Those mRNA isoforms with premature termination codons (PTCs) at least 50 bp upstream of an exon junction complex are likely to be targeted for degradation by the nonsense-mediated mRNA decay (NMD) pathway. Present in these mRNA isoforms are non-sense mediated RNA decay alternative exons (NSAE), which are alternatively spliced exons that lead to NMD. Mutations in traditional (BPS/PPT/3′ss/5′ss) and auxiliary splicing motifs may cause aberrant splicing, such as exon skipping or cryptic (or pseudo-) exon inclusion or splice-site activation, and contribute significantly to human morbidity and mortality. Both aberrant and alternative splicing patterns can be influenced by natural DNA variants in exons and introns.

### Target Transcripts

In some embodiments, the methods of the present disclosure exploit the presence of non-sense mediated RNA decay alternative exon-containing pre-mRNA (NSAE pre-mRNA) transcribed from a gene. In some embodiments, the methods of the present disclosure exploit the presence of non-sense mediated RNA decay alternative exon-containing pre-mRNA (NSAE pre-mRNA) transcribed from the SYNGAP1 gene. Canonical splicing of the identified SYNGAP1 NSAE pre-mRNA transcripts to produce functional, mature SYNGAP1 mRNA can be induced using a therapeutic agent, such as an ASO, that promotes constitutive splicing of SYNGAP1 NSAE pre-mRNA at the canonical splice sites. In some embodiments, the resulting functional, mature SYNGAP1 mRNA can be translated normally, thereby increasing the amount of functional SYNGAP1 protein in the patient's cells and preventing symptoms of SYNGAP1 associated disease. In some embodiments, canonical splicing of the identified SYNGAP1 NSAE pre-mRNA transcripts to produce functional, mature SYNGAP1 mRNA may be reduced using a therapeutic agent, such as an ASO, that inhibits constitutive splicing of SYNGAP1 NSAE pre-mRNA at the canonical splice sites. In some embodiments, the resulting functional, mature SYNGAP1 mRNA can be translated abnormally, thereby decreasing the amount of functional SYNGAP1 protein in the patient's cells and preventing symptoms of SYNGAP1 associated disease.

In some embodiments, the methods of the present disclosure exploit the presence of non-sense mediated RNA decay alternative exon-containing pre-mRNA (NSAE pre-mRNA) transcribed from the target gene, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. Canonical splicing of the identified target NSAE pre-mRNA transcripts to produce the functional, mature target mRNA can be induced using a therapeutic agent, such as an ASO, that promotes constitutive splicing of the target NSAE pre-mRNA at the canonical splice sites. In some embodiments, the resulting functional, mature target mRNA can be translated normally, thereby increasing the amount of the functional target protein in the patient's cells and preventing symptoms of the target associated disease. In some embodiments, canonical splicing of the identified target NSAE pre-mRNA transcripts to produce functional, mature target mRNA may be reduced using a therapeutic agent, such as an ASO, that inhibits constitutive splicing of target NSAE pre-mRNA at the canonical splice sites. In some embodiments, the resulting functional, mature target mRNA can be translated abnormally, thereby decreasing the amount of functional target protein in the patient's cells and preventing symptoms of the target associated disease.

In various embodiments, the present disclosure provides a therapeutic agent that can target SYNGAP1 pre-mRNA to modulate splicing or protein expression level. The therapeutic agent can be a small molecule, nucleic acid oligomer, or polypeptide. In some embodiments, the therapeutic agent is an ASO. Various regions or sequences on the SYNGAP1 pre-mRNA can be targeted by a therapeutic agent, such as an ASO. In some embodiments, the ASO targets a SYNGAP1 NSAE pre-mRNA transcribed from the SYNGAP1 gene. In some embodiments, the ASO targets a SYNGAP1 NSAE pre-mRNA transcribed from the SYNGAP1 gene comprising non-sense mediated RNA decay alternative exons (NSAEs). In some embodiments, the NSAE is comprises a portion of canonical exon 11 or the entire canonical exon 11 of a SYNGAP1 pre-mRNA transcript. In some embodiments, the NSAE is comprises only a portion of canonical exon 11 of a SYNGAP1 pre-mRNA transcript, or canonical exon 11 of a SYNGAP1 pre-mRNA transcript and at least a portion of the intron upstream of exon 11 of the SYNGAP1 pre-mRNA transcript. In some embodiments, the NSAE is included in a SYNGAP1 pre-mRNA transcript due to aberrant splicing. In some embodiments, the aberrant splicing is caused by a mutation in the SYNGAP1 gene. In some embodiments, the mutation is a G→A mutation at position −1 of the exon 11 splice donor (E8SJM, Exon 11 Splice Junction Mutation, c.894G>A). In some embodiments, the ASO targets a sequence within a NSAE of a SYNGAP1 pre-mRNA transcript. In some embodiments, the NSAE is comprises a portion of canonical exon 11 or the entire canonical exon 11 of a SYNGAP1 pre-mRNA transcript. In some embodiments, the ASO targets a sequence within exon 10 or 11 of a SYNGAP1 pre-mRNA transcript. In some embodiments, the ASO targets an exon sequence upstream (or 5′) from the 5′ splice site of exon 11 of a SYNGAP1 pre-mRNA transcript. In some embodiments, the ASO targets an exon sequence downstream (or 3′) from the 3′ splice site of exon 11 of a SYNGAP1 pre-mRNA transcript. In some embodiments, the ASO targets a sequence within an intron flanking the 3′ splice site of a NSAE of a SYNGAP1 pre-mRNA transcript. In some embodiments, the ASO targets a sequence within intron 10 or 11 of a SYNGAP1 pre-mRNA transcript. In some embodiments, the ASO targets an intron sequence upstream (or 5′) from the 3′ splice site of intron 10 or 11 of a SYNGAP1 pre-mRNA transcript. In some embodiments, the ASO targets an intron sequence downstream (or 3′) from the 5′ splice site of intron 10 or 11 of a SYNGAP1 pre-mRNA transcript. In some embodiments, the ASO targets a sequence within an intron flanking the 5′ splice site of a NSAE of a SYNGAP1 pre-mRNA transcript. In some embodiments, the ASO targets a sequence within intron 10 of a SYNGAP1 pre-mRNA transcript. In some embodiments, the ASO targets a sequence within intron 11 of a SYNGAP1 pre-mRNA transcript. In some embodiments, the ASO targets an intron sequence upstream (or 5′) from the 3′ splice site of intron 10 or 11 of a SYNGAP1 pre-mRNA transcript. In some embodiments, the ASO targets an intron sequence downstream (or 3′) from the 5′ splice site of intron 10 or 11 of a SYNGAP1 pre-mRNA transcript. In some embodiments, the ASO targets a sequence comprising an exon-intron boundary of a SYNGAP1 pre-mRNA transcript. In some embodiments, the exon is a NSAE. An exon-intron boundary can refer to the junction of an exon sequence and an intron sequence. In some embodiments, the intron sequence can flank the 5′ end of the NSAE, or the 3′ end of the exon. In some embodiments, the ASO targets a sequence comprising an exon 10-intron 11 boundary of a SYNGAP1 pre-mRNA transcript. In some embodiments, the ASO targets a sequence comprising an intron 10-exon 11 boundary of a SYNGAP1 NSAE pre-mRNA transcript. In some embodiments, the ASO targets a sequence comprising both a portion of an intron and a portion of an exon.

In various embodiments, the present disclosure provides a therapeutic agent that can target a target pre-mRNA to modulate splicing or protein expression level, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. The therapeutic agent can be a small molecule, nucleic acid oligomer, or polypeptide. In some embodiments, the therapeutic agent is an ASO. Various regions or sequences on the target pre-mRNA can be targeted by a therapeutic agent, such as an ASO. In some embodiments, the ASO targets a target NSAE pre-mRNA transcribed from the target gene. In some embodiments, the ASO targets a target NSAE pre-mRNA transcribed from the target gene comprising non-sense mediated RNA decay alternative exons (NSAEs). In some embodiments, the NSAE is comprises a portion of a canonical exon or the entire canonical exon of a target pre-mRNA transcript. In some embodiments, the NSAE is comprises only a portion of a canonical exon of a target pre-mRNA transcript, or a canonical exon of a target pre-mRNA transcript and at least a portion of the intron upstream of the exon of the target pre-mRNA transcript. In some embodiments, the NSAE is included in a target pre-mRNA transcript due to aberrant splicing. In some embodiments, the aberrant splicing is caused by a mutation in the target gene. In some embodiments, the ASO targets a sequence within a NSAE of a target pre-mRNA transcript. In some embodiments, the NSAE is comprises a portion of a canonical exon or the entire canonical exon of a target pre-mRNA transcript. In some embodiments, the ASO targets a sequence within a exon of a target pre-mRNA transcript. In some embodiments, the ASO targets an exon sequence upstream (or 5′) from the 5′ splice site of a exon of a target pre-mRNA transcript. In some embodiments, the ASO targets an exon sequence downstream (or 3′) from the 3′ splice site of a exon of a target pre-mRNA transcript. In some embodiments, the ASO targets a sequence within an intron flanking the 3′ splice site of a NSAE of a target pre-mRNA transcript. In some embodiments, the ASO targets a sequence within an intron of a target pre-mRNA transcript. In some embodiments, the ASO targets an intron sequence upstream (or 5′) from the 3′ splice site of an intron of a target pre-mRNA transcript. In some embodiments, the ASO targets an intron sequence downstream (or 3′) from the 5′ splice site of an intron of a target pre-mRNA transcript. In some embodiments, the ASO targets a sequence within an intron flanking the 5′ splice site of a NSAE of a target pre-mRNA transcript. In some embodiments, the ASO targets a sequence within an intron of a target pre-mRNA transcript. In some embodiments, the ASO targets an intron sequence upstream (or 5′) from the 3′ splice site of an intron of a target pre-mRNA transcript. In some embodiments, the ASO targets an intron sequence downstream (or 3′) from the 5′ splice site of an intron of a target pre-mRNA transcript. In some embodiments, the ASO targets a sequence comprising an exon-intron boundary of a target pre-mRNA transcript. In some embodiments, the exon is a NSAE. An exon-intron boundary can refer to the junction of an exon sequence and an intron sequence. In some embodiments, the intron sequence can flank the 5′ end of the NSAE, or the 3′ end of the exon. In some embodiments, the ASO targets a sequence comprising an exon-intron boundary of a target pre-mRNA transcript. In some embodiments, the ASO targets a sequence comprising an intron-exon boundary of a target NSAE pre-mRNA transcript. In some embodiments, the ASO targets a sequence comprising both a portion of an intron and a portion of an exon.

In some embodiments, the ASO targets a sequence about 4 to about 300 nucleotides upstream (or 5′) from the 3′ splice site of a NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 3′ splice site of the NSAE of a SYNGAP1 pre-mRNA transcript. In some embodiments, the ASO may target a sequence more than 300 nucleotides upstream from the 3′ splice site of the NSAE of a SYNGAP1pre-mRNA transcript. In some embodiments, the ASO targets a sequence about 4 to about 300 nucleotides downstream (or 3′) from the 3′ splice site of a NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream (or 3′) from the 3′ splice site of the NSAE of a SYNGAP1 pre-mRNA transcript. In some embodiments, the ASO may target a sequence more than 300 nucleotides downstream from the 3′ splice site of the NSAE of a SYNGAP1 pre-mRNA transcript. In some embodiments, the ASO targets a sequence about 4 to about 300 nucleotides downstream (or 3′) from the 5′ splice site of the NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 5′ splice site of the NSAE of a SYNGAP1 pre-mRNA transcript. In some embodiments, the ASO targets a sequence more than 300 nucleotides downstream from the 5′ splice site of the NSAE of a SYNGAP1 pre-mRNA transcript. In some embodiments, the ASO targets a sequence about 4 to about 300 nucleotides upstream (or 5′) from the 5′ splice site of the NSAE of a SYNGAP1 pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream from the 5′ splice site of the NSAE exon of a SYNGAP1 pre-mRNA transcript. In some embodiments, the ASO targets a sequence more than 300 nucleotides upstream from the 5′ splice site of the NSAE exon of a SYNGAP1 pre-mRNA transcript.

In some embodiments, the ASO targets a sequence about 4 to about 300 nucleotides upstream (or 5′) from the 3′ splice site of a NSAE of a target pre-mRNA transcript, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream (or 5′) from the 3′ splice site of the NSAE of a target pre-mRNA transcript. In some embodiments, the ASO may target a sequence more than 300 nucleotides upstream from the 3′ splice site of the NSAE of a target pre-mRNA transcript. In some embodiments, the ASO targets a sequence about 4 to about 300 nucleotides downstream (or 3′) from the 3′ splice site of a NSAE of a target pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream (or 3′) from the 3′ splice site of the NSAE of a target pre-mRNA transcript. In some embodiments, the ASO may target a sequence more than 300 nucleotides downstream from the 3′ splice site of the NSAE of a target pre-mRNA transcript. In some embodiments, the ASO targets a sequence about 4 to about 300 nucleotides downstream (or 3′) from the 5′ splice site of the NSAE of a target pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides downstream from the 5′ splice site of the NSAE of a target pre-mRNA transcript. In some embodiments, the ASO targets a sequence more than 300 nucleotides downstream from the 5′ splice site of the NSAE of a target pre-mRNA transcript. In some embodiments, the ASO targets a sequence about 4 to about 300 nucleotides upstream (or 5′) from the 5′ splice site of the NSAE of a target pre-mRNA transcript. In some embodiments, the ASO targets a sequence about 1 to about 20 nucleotides, about 20 to about 50 nucleotides, about 50 to about 100 nucleotides, about 100 to about 150 nucleotides, about 150 to about 200 nucleotides, about 200 to about 250 nucleotides, or about 250 to about 300 nucleotides upstream from the 5′ splice site of the NSAE exon of a target pre-mRNA transcript. In some embodiments, the ASO targets a sequence more than 300 nucleotides upstream from the 5′ splice site of the NSAE exon of a target pre-mRNA transcript.

In some embodiments, the ASOs disclosed herein target a NSAE pre-mRNA transcribed from a genomic sequence selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 genomic sequences. In some embodiments, the ASO targets a NSAE pre-mRNA transcript from a genomic sequence comprising a NSAE exon selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 genomic sequences. In some embodiments, the ASO targets a NSAE pre-mRNA transcript from a genomic sequence comprising an intron flanking the 3′ splice site of the NSAE exon and an intron flanking the 5′ splice site of a NSAE exon selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 genomic sequences. In some embodiments, the ASO targets a NSAE pre-mRNA transcript comprising a sequence selected from the group consisting of the pre-mRNA transcripts of Table 4. In some embodiments, the ASO targets a pre-mRNA sequence comprising a NSAE exon selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 pre-mRNA sequences. In some embodiments, the ASO targets a pre-mRNA sequence comprising an intron flanking the 3′ splice site of the NSAE exon selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 pre-mRNA sequences. In some embodiments, the ASO targets a pre-mRNA sequence comprising an intron flanking the 5′ splice site of the NSAE exon selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 pre-mRNA sequences. In some embodiments, the transcript is selected from the group consisting of the transcripts of Table 4.

In some embodiments, the pre-mRNA transcript comprises a sequence with at least about 80%, 85%, 90%, 95%, 96%, 97%, 98%, 99% or 100% sequence identity to a pre-mRNA transcript selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 pre-mRNA transcripts or a complement thereof described herein.

In some embodiments, the targeted portion of the pre-mRNA selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 pre-mRNAs comprises a sequence with at least 80%, 85%, 90%, 95%, 97%, or 100% sequence identity to a region comprising at least 8 contiguous nucleic acids of a sequence of the pre-mRNA transcripts of Table 4 or a sequence of Table 3 or complements thereof. In some embodiments, the targeted portion of the pre-mRNA selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 pre-mRNAs comprises a sequence with at least 80%, 85%, 90%, 95%, 97%, or 100% sequence identity to a region comprising at least 8 contiguous nucleic acids of a sequence selected from the group consisting of SEQ ID NO: 170-SEQ ID NO: 1098 or complements thereof. In some embodiments, the targeted portion of the pre-mRNA selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SYNGAP1 genomic sequence. In some embodiments, the ASO targets a NSAE pre-mRNA transcript from a SYNGAP1 genomic sequence comprising a NSAE exon. In some embodiments the NSAE exon is exon 11. In some embodiments, the ASO targets a NSAE pre-mRNA transcript from a SYNGAP1 genomic sequence comprising exon 11. In some embodiments, the ASO targets a NSAE pre-mRNA transcript from a SYNGAP1 genomic sequence comprising an intron flanking the 3′ splice site of the NSAE exon and an intron flanking the 5′ splice site of a NSAE exon. In some embodiments, the intron flanking the 3′ splice site of the NSAE exon is intron 10 and the intron flanking the 5′ splice site of a NSAE exon is intron 11. In some embodiments, the ASO targets a NSAE pre-mRNA transcript from a SYNGAP1 genomic sequence comprising intron 10, exon 11 and intron 11. In some embodiments, the ASO targets a NSAE pre-mRNA transcript comprising a sequence selected from the group consisting of premrna_ENST00000293748.9, premrna_ENST00000418600.7, premrna_ENST00000428982.4, premrna_ENST00000449372.7, premrna_ENST00000470232.1, premrna_ENST00000479510.2, premrna_ENST00000628646.2, premrna_ENST00000629380.3, premrna_ENST00000635885.1, premrna_ENST00000636075.1, premrna_ENST00000636116.1, premrna_ENST00000636146.1, premrna_ENST00000636193.1, premrna_ENST00000636436.1, premrna_ENST00000636443.1, premrna_ENST00000636640.1, premrna_ENST00000636731.1, premrna_ENST00000636905.1, premrna_ENST00000637052.1, premrna_ENST00000637194.1, premrna_ENST00000637490.1, premrna_ENST00000637587.1, premrna_ENST00000637671.1, premrna_ENST00000637721.1, premrna_ENST00000637911.1, premrna_ENST00000638127.1, premrna_ENST00000638142.2, premrna_ENST00000644458.1, premrna_ENST00000645250.1, premrna_ENST00000646630.1. In some embodiments, the ASO targets a NSAE pre-mRNA transcript comprising exon 10 and exon 11. In some embodiments, the ASO targets a SYNGAP1 pre-mRNA sequence comprising a NSAE exon. In some embodiments, the ASO targets a SYNGAP1 pre-mRNA sequence comprising exon 11. In some embodiments, the ASO targets a SYNGAP1 pre-mRNA sequence comprising an intron flanking the 3′ splice site of the NSAE exon. In some embodiments, the ASO targets a SYNGAP1 pre-mRNA sequence comprising intron 10. In some embodiments, the ASO targets a SYNGAP1 pre-mRNA sequence comprising an intron flanking the 5′ splice site of the NSAE exon. In some embodiments, the transcript is selected from the group consisting of transcript_ENST00000293748.9, transcript_ENST00000418600.7, transcript_ENST00000428982.4, transcript_ENST00000449372.7, transcript_ENST00000470232.1, transcript_ENST00000479510.2, transcript_ENST00000628646.2, transcript_ENST00000629380.3, transcript_ENST00000635885.1, transcript_ENST00000636075.1, transcript_ENST00000636116.1, transcript_ENST00000636146.1, transcript_ENST00000636193.1, transcript_ENST00000636436.1, transcript_ENST00000636443.1, transcript_ENST00000636640.1, transcript_ENST00000636731.1, transcript_ENST00000636905.1, transcript_ENST00000637052.1, transcript_ENST00000637194.1, transcript_ENST00000637490.1, transcript_ENST00000637587.1, transcript_ENST00000637671.1, transcript_ENST00000637721.1, transcript_ENST00000637911.1, transcript_ENST00000638127.1, transcript_ENST00000638142.2, transcript_ENST00000644458.1, transcript_ENST00000645250.1 and transcript_ENST00000646630.1.

In some embodiments, the SYNGAP1 pre-mRNA transcript is encoded by a genetic sequence with at least about 80%, 85%, 90%, 95%, 96%, 97%, 98%, 99% or 100% sequence identity to the Ensembl reference number ENSG00000227460.8 or ENSG00000197283.17 or a complement thereof. In some embodiments, the SYNGAP1 pre-mRNA transcript comprises a sequence with at least about 80%, 85%, 90%, 95%, 96%, 97%, 98%, 99% or 100% sequence identity to a SYNGAP1 pre-mRNA transcript or a complement thereof described herein.

In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence of premrna_ENST00000293748.9, premrna_ENST00000418600.7, premrna_ENST00000428982.4, premrna_ENST00000449372.7, premrna_ENST00000470232.1, premrna_ENST00000479510.2, premrna_ENST00000628646.2, premrna_ENST00000629380.3, premrna_ENST00000635885.1, premrna_ENST00000636075.1, premrna_ENST00000636116.1, premrna_ENST00000636146.1, premrna_ENST00000636193.1, premrna_ENST00000636436.1, premrna_ENST00000636443.1, premrna_ENST00000636640.1, premrna_ENST00000636731.1, premrna_ENST00000636905.1, premrna_ENST00000637052.1, premrna_ENST00000637194.1, premrna_ENST00000637490.1, premrna_ENST00000637587.1, premrna_ENST00000637671.1, premrna_ENST00000637721.1, premrna_ENST00000637911.1, premrna_ENST00000638127.1, premrna_ENST00000638142.2, premrna_ENST00000644458.1, premrna_ENST00000645250.1, premrna_ENST00000646630.1 or a sequence of Table 2 or complements thereof. In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 166-SEQ ID NO: 169 or complements thereof. In some embodiments, the ASO comprises a sequence that is at least about 80%, 85%, 90%, 95%, 97%, or 100% identical to any one the sequences of Table 1 or complements thereof.

In some embodiments, the ASOs disclosed herein target a NSAE pre-mRNA transcribed from a ABCA5 genomic sequence. In some embodiments, the ASO targets a NSAE pre-mRNA transcript from a ABCA5 genomic sequence comprising a NSAE exon. In some embodiments, the ASO targets a NSAE pre-mRNA transcript from a ABCA5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000392676.8, premrna_ENST00000586601.1, premrna_ENST00000586811.1, premrna_ENST00000586995.5, premrna_ENST00000587607.5, premrna_ENST00000588106.1, premrna_ENST00000588665.5, premrna_ENST00000588877.5, premrna_ENST00000589609.1, premrna_ENST00000589975.5, premrna_ENST00000591234.5, premrna_ENST00000592568.1, premrna_ENST00000593153.5, and premrna_ENST00000593253.5. In some embodiments, the ASO targets a ABCA5 pre-mRNA sequence comprising a NSAE exon. In some embodiments, the ASO targets a ABCA5 pre-mRNA sequence comprising an intron flanking the 3′ splice site of the NSAE exon. In some embodiments, the ASO targets a ABCA5 pre-mRNA sequence comprising an intron flanking the 5′ splice site of the NSAE exon. In some embodiments, the transcript is selected from the group consisting of transcript_ENST00000392676.8, transcript_ENST00000586601.1, transcript_ENST00000586811.1, transcript_ENST00000586995.5, transcript_ENST00000587607.5, transcript_ENST00000588106.1, transcript_ENST00000588665.5, transcript_ENST00000588877.5, transcript_ENST00000589609.1, transcript_ENST00000589975.5, transcript_ENST00000591234.5, transcript_ENST00000592568.1, transcript_ENST00000593153.5, and transcript_ENST00000593253.5.

In some embodiments, the ABCA5 pre-mRNA transcript is encoded by a genetic sequence with at least about 80%, 85%, 90%, 95%, 96%, 97%, 98%, 99% or 100% sequence identity to the Ensembl reference number ENSG00000154265.16 or a complement thereof. In some embodiments, the ABCA5 pre-mRNA transcript comprises a sequence with at least about 80%, 85%, 90%, 95%, 96%, 97%, 98%, 99% or 100% sequence identity to a ABCA5 pre-mRNA transcript or a complement thereof described herein.

In some embodiments, the targeted portion of the ABCA5 pre-mRNA comprises a sequence with at least 80%, 85%, 90%, 95%, 97%, or 100% sequence identity to a region comprising at least 8 contiguous nucleic acids of a sequence of premrna_ENST00000392676.8, premrna_ENST00000586601.1, premrna_ENST00000586811.1, premrna_ENST00000586995.5, premrna_ENST00000587607.5, premrna_ENST00000588106.1, premrna_ENST00000588665.5, premrna_ENST00000588877.5, premrna_ENST00000589609.1, premrna_ENST00000589975.5, premrna_ENST00000591234.5, premrna_ENST00000592568.1, premrna_ENST00000593153.5, premrna_ENST00000593253.5, or a sequence of Table 3 or complements thereof. In some embodiments, the targeted portion of the ABCA5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 170-SEQ ID NO: 173 or complements thereof. In some embodiments, the targeted portion of the ABCA5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ABCA7 genomic sequence. In some embodiments, the ASO targets a NSAE pre-mRNA transcript from a ABCA7 genomic sequence comprising a NSAE exon. In some embodiments, the ASO targets a NSAE pre-mRNA transcript from a ABCA7 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000263094.11, premrna_ENST00000433129.6, premrna_ENST00000435683.7, premrna_ENST00000524383.1, premrna_ENST00000524850.5, premrna_ENST00000525073.6, premrna_ENST00000525238.2, premrna_ENST00000526885.5, premrna_ENST00000527496.1, premrna_ENST00000529442.7, premrna_ENST00000530092.2, premrna_ENST00000530703.1, premrna_ENST00000531467.5, premrna_ENST00000531478.5, premrna_ENST00000532194.3, premrna_ENST00000533574.1, premrna_ENST00000612569.1, and premrna_ENST00000673773.1. In some embodiments, the ASO targets a ABCA7 pre-mRNA sequence comprising a NSAE exon. In some embodiments, the ASO targets a ABCA7 pre-mRNA sequence comprising an intron flanking the 3′ splice site of the NSAE exon. In some embodiments, the ASO targets a ABCA7 pre-mRNA sequence comprising an intron flanking the 5′ splice site of the NSAE exon. In some embodiments, the transcript is selected from the group consisting of transcript_ENST00000263094.11, transcript_ENST00000433129.6, transcript_ENST00000435683.7, transcript_ENST00000524383.1, transcript_ENST00000524850.5, transcript_ENST00000525073.6, transcript_ENST00000525238.2, transcript_ENST00000526885.5, transcript_ENST00000527496.1, transcript_ENST00000529442.7, transcript_ENST00000530092.2, transcript_ENST00000530703.1, transcript_ENST00000531467.5, transcript_ENST00000531478.5, transcript_ENST00000532194.3, transcript_ENST00000533574.1, transcript_ENST00000612569.1, and transcript_ENST00000673773.1.

In some embodiments, the ABCA7 pre-mRNA transcript is encoded by a genetic sequence with at least about 80%, 85%, 90%, 95%, 96%, 97%, 98%, 99% or 100% sequence identity to the Ensembl reference number ENSG00000064687.13 or a complement thereof. In some embodiments, the ABCA7 pre-mRNA transcript comprises a sequence with at least about 80%, 85%, 90%, 95%, 96%, 97%, 98%, 99% or 100% sequence identity to a ABCA7 pre-mRNA transcript or a complement thereof described herein.

In some embodiments, the targeted portion of the ABCA7 pre-mRNA comprises a sequence with at least 80%, 85%, 90%, 95%, 97%, or 100% sequence identity to a region comprising at least 8 contiguous nucleic acids of a sequence of premrna_ENST00000263094.11, premrna_ENST00000433129.6, premrna_ENST00000435683.7, premrna_ENST00000524383.1, premrna_ENST00000524850.5, premrna_ENST00000525073.6, premrna_ENST00000525238.2, premrna_ENST00000526885.5, premrna_ENST00000527496.1, premrna_ENST00000529442.7, premrna_ENST00000530092.2, premrna_ENST00000530703.1, premrna_ENST00000531467.5, premrna_ENST00000531478.5, premrna_ENST00000532194.3, premrna_ENST00000533574.1, premrna_ENST00000612569.1, premrna_ENST00000673773.1 or a sequence of Table 3 or complements thereof. In some embodiments, the targeted portion of the ABCA7 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 174-SEQ ID NO: 177 or complements thereof. In some embodiments, the targeted portion of the ABCA7 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ABCC3 genomic sequence. In some embodiments, the ASO targets a NSAE pre-mRNA transcript from a ABCC3 genomic sequence comprising a NSAE exon. In some embodiments, the ASO targets a NSAE pre-mRNA transcript from a ABCC3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000285238.13, premrna_ENST00000427699.5, premrna_ENST00000502426.5, premrna_ENST00000503304.1, premrna_ENST00000503337.1, premrna_ENST00000504586.1, premrna_ENST00000505699.5, premrna_ENST00000506464.5, premrna_ENST00000508929.1, premrna_ENST00000510633.5, premrna_ENST00000510891.1, premrna_ENST00000513511.5, premrna_ENST00000513589.1, premrna_ENST00000513745.1, premrna_ENST00000515070.1, premrna_ENST00000515585.1, premrna_ENST00000515707.1, and premrna_ENST00000571855.1 In some embodiments, the ASO targets a ABCC3 pre-mRNA sequence comprising a NSAE exon. In some embodiments, the ASO targets a ABCC3 pre-mRNA sequence comprising an intron flanking the 3′ splice site of the NSAE exon. In some embodiments, the ASO targets a ABCC3 pre-mRNA sequence comprising an intron flanking the 5′ splice site of the NSAE exon. In some embodiments, the transcript is selected from the group consisting of transcript_ENST00000285238.13, transcript_ENST00000427699.5, transcript_ENST00000502426.5, transcript_ENST00000503304.1, transcript_ENST00000503337.1, transcript_ENST00000504586.1, transcript_ENST00000505699.5, transcript_ENST00000506464.5, transcript_ENST00000508929.1, transcript_ENST00000510633.5, transcript_ENST00000510891.1, transcript_ENST00000513511.5, transcript_ENST00000513589.1, transcript_ENST00000513745.1, transcript_ENST00000515070.1, transcript_ENST00000515585.1, transcript_ENST00000515707.1, and transcript_ENST00000571855.1.

In some embodiments, the ABCC3 pre-mRNA transcript is encoded by a genetic sequence with at least about 80%, 85%, 90%, 95%, 96%, 97%, 98%, 99% or 100% sequence identity to the Ensembl reference number ENSG00000108846.16 or a complement thereof. In some embodiments, the ABCC3 pre-mRNA transcript comprises a sequence with at least about 80%, 85%, 90%, 95%, 96%, 97%, 98%, 99% or 100% sequence identity to a ABCC3 pre-mRNA transcript or a complement thereof described herein.

In some embodiments, the targeted portion of the ABCC3 pre-mRNA comprises a sequence with at least 80%, 85%, 90%, 95%, 97%, or 100% sequence identity to a region comprising at least 8 contiguous nucleic acids of a sequence of premrna_ENST00000285238.13, premrna_ENST00000427699.5, premrna_ENST00000502426.5, premrna_ENST00000503304.1, premrna_ENST00000503337.1, premrna_ENST00000504586.1, premrna_ENST00000505699.5, premrna_ENST00000506464.5, premrna_ENST00000508929.1, premrna_ENST00000510633.5, premrna_ENST00000510891.1, premrna_ENST00000513511.5, premrna_ENST00000513589.1, premrna_ENST00000513745.1, premrna_ENST00000515070.1, premrna_ENST00000515585.1, premrna_ENST00000515707.1, premrna_ENST00000571855.1 or a sequence of Table 3 or complements thereof. In some embodiments, the targeted portion of the ABCC3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 178-SEQ ID NO: 181 or complements thereof. In some embodiments, the targeted portion of the ABCC3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ABCC5 genomic sequence. In some embodiments, the ASO targets a NSAE pre-mRNA transcript from a ABCC5 genomic sequence comprising a NSAE exon. In some embodiments, the ASO targets a NSAE pre-mRNA transcript from a ABCC5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000265586.10, premrna_ENST00000334444.11, premrna_ENST00000382494.6, premrna_ENST00000392579.6, premrna_ENST00000427120.6, premrna_ENST00000437205.5, premrna_ENST00000437341.5, premrna_ENST00000438979.6, premrna_ENST00000443376.5, premrna_ENST00000443497.1, premrna_ENST00000446941.2, premrna_ENST00000476402.1, and premrna_ENST00000492216.1. In some embodiments, the ASO targets a ABCC5 pre-mRNA sequence comprising a NSAE exon. In some embodiments, the ASO targets a ABCC5 pre-mRNA sequence comprising an intron flanking the 3′ splice site of the NSAE exon. In some embodiments, the ASO targets a ABCC5 pre-mRNA sequence comprising an intron flanking the 5′ splice site of the NSAE exon. In some embodiments, the transcript is selected from the group consisting of transcript_ENST00000265586.10, transcript_ENST00000334444.11, transcript_ENST00000382494.6, transcript_ENST00000392579.6, transcript_ENST00000427120.6, transcript_ENST00000437205.5, transcript_ENST00000437341.5, transcript_ENST00000438979.6, transcript_ENST00000443376.5, transcript_ENST00000443497.1, transcript_ENST00000446941.2, transcript_ENST00000476402.1, and transcript_ENST00000492216.1.

In some embodiments, the ABCC5 pre-mRNA transcript is encoded by a genetic sequence with at least about 80%, 85%, 90%, 95%, 96%, 97%, 98%, 99% or 100% sequence identity to the Ensembl reference number ENSG00000114770.17 or a complement thereof. In some embodiments, the ABCC5 pre-mRNA transcript comprises a sequence with at least about 80%, 85%, 90%, 95%, 96%, 97%, 98%, 99% or 100% sequence identity to a ABCC5 pre-mRNA transcript or a complement thereof described herein.

In some embodiments, the targeted portion of the ABCC5 pre-mRNA comprises a sequence with at least 80%, 85%, 90%, 95%, 97%, or 100% sequence identity to a region comprising at least 8 contiguous nucleic acids of a sequence of premrna_ENST00000265586.10, premrna_ENST00000334444.11, premrna_ENST00000382494.6, premrna_ENST00000392579.6, premrna_ENST00000427120.6, premrna_ENST00000437205.5, premrna_ENST00000437341.5, premrna_ENST00000438979.6, premrna_ENST00000443376.5, premrna_ENST00000443497.1, premrna_ENST00000446941.2, premrna_ENST00000476402.1, premrna_ENST00000492216.1 or a sequence of Table 3 or complements thereof. In some embodiments, the targeted portion of the ABCC5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 182-SEQ ID NO: 185 or complements thereof. In some embodiments, the targeted portion of the ABCC5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ABCC8 genomic sequence. In some embodiments, the ASO targets a NSAE pre-mRNA transcript from a ABCC8 genomic sequence comprising a NSAE exon. In some embodiments, the ASO targets a NSAE pre-mRNA transcript from a ABCC8 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000302539.9, premrna_ENST00000389817.7, premrna_ENST00000524561.1, premrna_ENST00000525022.1, premrna_ENST00000526002.1, premrna_ENST00000526037.5, premrna_ENST00000526168.5, premrna_ENST00000526921.5, premrna_ENST00000527905.5, premrna_ENST00000528202.5, premrna_ENST00000528374.1, premrna_ENST00000529967.5, premrna_ENST00000530147.5, premrna_ENST00000531137.1, premrna_ENST00000531642.5, premrna_ENST00000531891.1, premrna_ENST00000531911.1, premrna_ENST00000532220.1, premrna_ENST00000532728.6, premrna_ENST00000612903.1, premrna_ENST00000635881.1, premrna_ENST00000642271.1, premrna_ENST00000642579.1, premrna_ENST00000642611.1, premrna_ENST00000642902.1, premrna_ENST00000643260.1, premrna_ENST00000643562.1, premrna_ENST00000643925.1, premrna_ENST00000644057.1, premrna_ENST00000644447.1, premrna_ENST00000644472.1, premrna_ENST00000644484.1, premrna_ENST00000644542.1, premrna_ENST00000644649.1, premrna_ENST00000644675.1, premrna_ENST00000644757.1, premrna_ENST00000644772.1, premrna_ENST00000645004.1, premrna_ENST00000645076.1, premrna_ENST00000645417.1, premrna_ENST00000645744.1, premrna_ENST00000645760.1, premrna_ENST00000645884.1, premrna_ENST00000646003.1, premrna_ENST00000646207.1, premrna_ENST00000646276.1, premrna_ENST00000646592.1, premrna_ENST00000646737.1, premrna_ENST00000646902.1, premrna_ENST00000646993.1, premrna_ENST00000647013.1, premrna_ENST00000647015.1, premrna_ENST00000647086.1, and premrna_ENST00000647158.1. In some embodiments, the ASO targets a ABCC8 pre-mRNA sequence comprising a NSAE exon. In some embodiments, the ASO targets a ABCC8 pre-mRNA sequence comprising an intron flanking the 3′ splice site of the NSAE exon. In some embodiments, the ASO targets a ABCC8 pre-mRNA sequence comprising an intron flanking the 5′ splice site of the NSAE exon. In some embodiments, the transcript is selected from the group consisting of transcript_ENST00000302539.9, transcript_ENST00000389817.7, transcript_ENST00000524561.1, transcript_ENST00000525022.1, transcript_ENST00000526002.1, transcript_ENST00000526037.5, transcript_ENST00000526168.5, transcript_ENST00000526921.5, transcript_ENST00000527905.5, transcript_ENST00000528202.5, transcript_ENST00000528374.1, transcript_ENST00000529967.5, transcript_ENST00000530147.5, transcript_ENST00000531137.1, transcript_ENST00000531642.5, transcript_ENST00000531891.1, transcript_ENST00000531911.1, transcript_ENST00000532220.1, transcript_ENST00000532728.6, transcript_ENST00000612903.1, transcript_ENST00000635881.1, transcript_ENST00000642271.1, transcript_ENST00000642579.1, transcript_ENST00000642611.1, transcript_ENST00000642902.1, transcript_ENST00000643260.1, transcript_ENST00000643562.1, transcript_ENST00000643925.1, transcript_ENST00000644057.1, transcript_ENST00000644447.1, transcript_ENST00000644472.1, transcript_ENST00000644484.1, transcript_ENST00000644542.1, transcript_ENST00000644649.1, transcript_ENST00000644675.1, transcript_ENST00000644757.1, transcript_ENST00000644772.1, transcript_ENST00000645004.1, transcript_ENST00000645076.1, transcript_ENST00000645417.1, transcript_ENST00000645744.1, transcript_ENST00000645760.1, transcript_ENST00000645884.1, transcript_ENST00000646003.1, transcript_ENST00000646207.1, transcript_ENST00000646276.1, transcript_ENST00000646592.1, transcript_ENST00000646737.1, transcript_ENST00000646902.1, transcript_ENST00000646993.1, transcript_ENST00000647013.1, transcript_ENST00000647015.1, transcript_ENST00000647086.1, and transcript_ENST00000647158.1.

In some embodiments, the ABCC8 pre-mRNA transcript is encoded by a genetic sequence with at least about 80%, 85%, 90%, 95%, 96%, 97%, 98%, 99% or 100% sequence identity to the Ensembl reference number ENSG00000006071.15 or a complement thereof. In some embodiments, the ABCC8 pre-mRNA transcript comprises a sequence with at least about 80%, 85%, 90%, 95%, 96%, 97%, 98%, 99% or 100% sequence identity to a ABCC8 pre-mRNA transcript or a complement thereof described herein.

In some embodiments, the targeted portion of the ABCC8 pre-mRNA comprises a sequence with at least 80%, 85%, 90%, 95%, 97%, or 100% sequence identity to a region comprising at least 8 contiguous nucleic acids of a sequence of premrna_ENST00000302539.9, premrna_ENST00000389817.7, premrna_ENST00000524561.1, premrna_ENST00000525022.1, premrna_ENST00000526002.1, premrna_ENST00000526037.5, premrna_ENST00000526168.5, premrna_ENST00000526921.5, premrna_ENST00000527905.5, premrna_ENST00000528202.5, premrna_ENST00000528374.1, premrna_ENST00000529967.5, premrna_ENST00000530147.5, premrna_ENST00000531137.1, premrna_ENST00000531642.5, premrna_ENST00000531891.1, premrna_ENST00000531911.1, premrna_ENST00000532220.1, premrna_ENST00000532728.6, premrna_ENST00000612903.1, premrna_ENST00000635881.1, premrna_ENST00000642271.1, premrna_ENST00000642579.1, premrna_ENST00000642611.1, premrna_ENST00000642902.1, premrna_ENST00000643260.1, premrna_ENST00000643562.1, premrna_ENST00000643925.1, premrna_ENST00000644057.1, premrna_ENST00000644447.1, premrna_ENST00000644472.1, premrna_ENST00000644484.1, premrna_ENST00000644542.1, premrna_ENST00000644649.1, premrna_ENST00000644675.1, premrna_ENST00000644757.1, premrna_ENST00000644772.1, premrna_ENST00000645004.1, premrna_ENST00000645076.1, premrna_ENST00000645417.1, premrna_ENST00000645744.1, premrna_ENST00000645760.1, premrna_ENST00000645884.1, premrna_ENST00000646003.1, premrna_ENST00000646207.1, premrna_ENST00000646276.1, premrna_ENST00000646592.1, premrna_ENST00000646737.1, premrna_ENST00000646902.1, premrna_ENST00000646993.1, premrna_ENST00000647013.1, premrna_ENST00000647015.1, premrna_ENST00000647086.1, premrna_ENST00000647158.1 or a sequence of Table 3 or complements thereof. In some embodiments, the targeted portion of the ABCC8 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 186-SEQ ID NO: 189 or complements thereof. In some embodiments, the targeted portion of the ABCC8 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ABCD1 genomic sequence. In some embodiments, the ASO targets a NSAE pre-mRNA transcript from a ABCD1 genomic sequence comprising a NSAE exon. In some embodiments, the ASO targets a NSAE pre-mRNA transcript from a ABCD1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000218104.6, premrna_ENST00000370129.4, and premrna_ENST00000443684.2. In some embodiments, the ASO targets a ABCD1 pre-mRNA sequence comprising a NSAE exon. In some embodiments, the ASO targets a ABCD1 pre-mRNA sequence comprising an intron flanking the 3′ splice site of the NSAE exon. In some embodiments, the ASO targets a ABCD1 pre-mRNA sequence comprising an intron flanking the 5′ splice site of the NSAE exon. In some embodiments, the transcript is selected from the group consisting of transcript_ENST00000218104.6, transcript_ENST00000370129.4, and transcript_ENST00000443684.2.

In some embodiments, the ABCD1 pre-mRNA transcript is encoded by a genetic sequence with at least about 80%, 85%, 90%, 95%, 96%, 97%, 98%, 99% or 100% sequence identity to the Ensembl reference number ENSG00000101986.12 or a complement thereof. In some embodiments, the ABCD1 pre-mRNA transcript comprises a sequence with at least about 80%, 85%, 90%, 95%, 96%, 97%, 98%, 99% or 100% sequence identity to a ABCD1 pre-mRNA transcript or a complement thereof described herein.

In some embodiments, the targeted portion of the ABCD1 pre-mRNA comprises a sequence with at least 80%, 85%, 90%, 95%, 97%, or 100% sequence identity to a region comprising at least 8 contiguous nucleic acids of a sequence of premrna_ENST00000218104.6, premrna_ENST00000370129.4, premrna_ENST00000443684.2 or a sequence of Table 3 or complements thereof. In some embodiments, the targeted portion of the ABCD1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 190-SEQ ID NO: 193 or complements thereof. In some embodiments, the targeted portion of the ABCD1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ABR genomic sequence. In some embodiments, the ASO targets a NSAE pre-mRNA transcript from a ABR genomic sequence comprising a NSAE exon. In some embodiments, the ASO targets a NSAE pre-mRNA transcript from a ABR genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000291107.6, premrna_ENST00000302538.9, premrna_ENST00000536794.6, premrna_ENST00000543210.6, premrna_ENST00000544583.6, premrna_ENST00000570441.5, premrna_ENST00000570525.5, premrna_ENST00000570688.1, premrna_ENST00000571022.5, premrna_ENST00000571120.5, premrna_ENST00000571306.1, premrna_ENST00000571383.5, premrna_ENST00000571543.1, premrna_ENST00000571797.5, premrna_ENST00000571945.5, premrna_ENST00000572152.5, premrna_ENST00000572441.5, premrna_ENST00000572585.5, premrna_ENST00000572650.1, premrna_ENST00000573325.1, premrna_ENST00000573559.3, premrna_ENST00000573667.1, premrna_ENST00000573895.1, premrna_ENST00000574048.2, premrna_ENST00000574139.6, premrna_ENST00000574257.5, premrna_ENST00000574266.1, premrna_ENST00000574437.5, premrna_ENST00000574544.1, premrna_ENST00000574632.5, premrna_ENST00000574875.1, premrna_ENST00000575770.5, premrna_ENST00000575934.5, premrna_ENST00000576668.1, premrna_ENST00000576964.5, and premrna_ENST00000577052.5. In some embodiments, the ASO targets a ABR pre-mRNA sequence comprising a NSAE exon. In some embodiments, the ASO targets a ABR pre-mRNA sequence comprising an intron flanking the 3′ splice site of the NSAE exon. In some embodiments, the ASO targets a ABR pre-mRNA sequence comprising an intron flanking the 5′ splice site of the NSAE exon. In some embodiments, the transcript is selected from the group consisting of transcript_ENST00000291107.6, transcript_ENST00000302538.9, transcript_ENST00000536794.6, transcript_ENST00000543210.6, transcript_ENST00000544583.6, transcript_ENST00000570441.5, transcript_ENST00000570525.5, transcript_ENST00000570688.1, transcript_ENST00000571022.5, transcript_ENST00000571120.5, transcript_ENST00000571306.1, transcript_ENST00000571383.5, transcript_ENST00000571543.1, transcript_ENST00000571797.5, transcript_ENST00000571945.5, transcript_ENST00000572152.5, transcript_ENST00000572441.5, transcript_ENST00000572585.5, transcript_ENST00000572650.1, transcript_ENST00000573325.1, transcript_ENST00000573559.3, transcript_ENST00000573667.1, transcript_ENST00000573895.1, transcript_ENST00000574048.2, transcript_ENST00000574139.6, transcript_ENST00000574257.5, transcript_ENST00000574266.1, transcript_ENST00000574437.5, transcript_ENST00000574544.1, transcript_ENST00000574632.5, transcript_ENST00000574875.1, transcript_ENST00000575770.5, transcript_ENST00000575934.5, transcript_ENST00000576668.1, transcript_ENST00000576964.5, and transcript_ENST00000577052.5.

In some embodiments, the ABR pre-mRNA transcript is encoded by a genetic sequence with at least about 80%, 85%, 90%, 95%, 96%, 97%, 98%, 99% or 100% sequence identity to the Ensembl reference number ENSG00000276016.4, ENSG00000278741.4 or ENSG00000159842.15 or a complement thereof. In some embodiments, the ABR pre-mRNA transcript comprises a sequence with at least about 80%, 85%, 90%, 95%, 96%, 97%, 98%, 99% or 100% sequence identity to a ABR pre-mRNA transcript or a complement thereof described herein.

In some embodiments, the targeted portion of the ABR pre-mRNA comprises a sequence with at least 80%, 85%, 90%, 95%, 97%, or 100% sequence identity to a region comprising at least 8 contiguous nucleic acids of a sequence of premrna_ENST00000291107.6, premrna_ENST00000302538.9, premrna_ENST00000536794.6, premrna_ENST00000543210.6, premrna_ENST00000544583.6, premrna_ENST00000570441.5, premrna_ENST00000570525.5, premrna_ENST00000570688.1, premrna_ENST00000571022.5, premrna_ENST00000571120.5, premrna_ENST00000571306.1, premrna_ENST00000571383.5, premrna_ENST00000571543.1, premrna_ENST00000571797.5, premrna_ENST00000571945.5, premrna_ENST00000572152.5, premrna_ENST00000572441.5, premrna_ENST00000572585.5, premrna_ENST00000572650.1, premrna_ENST00000573325.1, premrna_ENST00000573559.3, premrna_ENST00000573667.1, premrna_ENST00000573895.1, premrna_ENST00000574048.2, premrna_ENST00000574139.6, premrna_ENST00000574257.5, premrna_ENST00000574266.1, premrna_ENST00000574437.5, premrna_ENST00000574544.1, premrna_ENST00000574632.5, premrna_ENST00000574875.1, premrna_ENST00000575770.5, premrna_ENST00000575934.5, premrna_ENST00000576668.1, premrna_ENST00000576964.5, premrna_ENST00000577052.5 or a sequence of Table 3 or complements thereof. In some embodiments, the targeted portion of the ABR pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 194-SEQ ID NO: 201 or complements thereof. In some embodiments, the targeted portion of the ABR pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ACAD9 genomic sequence. In some embodiments, the ASO targets a NSAE pre-mRNA transcript from a ACAD9 genomic sequence comprising a NSAE exon. In some embodiments, the ASO targets a NSAE pre-mRNA transcript from a ACAD9 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000308982.12, premrna_ENST00000505192.5, premrna_ENST00000505602.1, premrna_ENST00000505867.5, premrna_ENST00000508971.1, premrna_ENST00000511227.5, premrna_ENST00000511325.1, premrna_ENST00000511526.5, premrna_ENST00000512801.5, premrna_ENST00000514336.1, premrna_ENST00000514643.5, and premrna_ENST00000515429.1. In some embodiments, the ASO targets a ACAD9 pre-mRNA sequence comprising a NSAE exon. In some embodiments, the ASO targets a ACAD9 pre-mRNA sequence comprising an intron flanking the 3′ splice site of the NSAE exon. In some embodiments, the ASO targets a ACAD9 pre-mRNA sequence comprising an intron flanking the 5′ splice site of the NSAE exon. In some embodiments, the transcript is selected from the group consisting of transcript_ENST00000308982.12, transcript_ENST00000505192.5, transcript_ENST00000505602.1, transcript_ENST00000505867.5, transcript_ENST00000508971.1, transcript_ENST00000511227.5, transcript_ENST00000511325.1, transcript_ENST00000511526.5, transcript_ENST00000512801.5, transcript_ENST00000514336.1, transcript_ENST00000514643.5, and transcript_ENST00000515429.1.

In some embodiments, the ACAD9 pre-mRNA transcript is encoded by a genetic sequence with at least about 80%, 85%, 90%, 95%, 96%, 97%, 98%, 99% or 100% sequence identity to the Ensembl reference number ENSG00000177646.19 or a complement thereof. In some embodiments, the ACAD9 pre-mRNA transcript comprises a sequence with at least about 80%, 85%, 90%, 95%, 96%, 97%, 98%, 99% or 100% sequence identity to a ACAD9 pre-mRNA transcript or a complement thereof described herein.

In some embodiments, the targeted portion of the ACAD9 pre-mRNA comprises a sequence with at least 80%, 85%, 90%, 95%, 97%, or 100% sequence identity to a region comprising at least 8 contiguous nucleic acids of a sequence of premrna_ENST00000308982.12, premrna_ENST00000505192.5, premrna_ENST00000505602.1, premrna_ENST00000505867.5, premrna_ENST00000508971.1, premrna_ENST00000511227.5, premrna_ENST00000511325.1, premrna_ENST00000511526.5, premrna_ENST00000512801.5, premrna_ENST00000514336.1, premrna_ENST00000514643.5, premrna_ENST00000515429.1 or a sequence of Table 3 or complements thereof. In some embodiments, the targeted portion of the ACAD9 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 202-SEQ ID NO: 207 or complements thereof. In some embodiments, the targeted portion of the ACAD9 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ACAP1 genomic sequence. In some embodiments, the ASO targets a NSAE pre-mRNA transcript from a ACAP1 genomic sequence comprising a NSAE exon. In some embodiments, the ASO targets a NSAE pre-mRNA transcript from a ACAP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000308982.12, premrna_ENST00000505192.5, premr-na_ENST00000505602.1, premrna_ENST00000505867.5, premrna_ENST00000508971.1, premr-na_ENST00000511227.5, premrna_ENST00000511325.1, premrna_ENST00000511526.5, premr-na_ENST00000512801.5, premrna_ENST00000514336.1, premrna_ENST00000514643.5, and premrna_ENST00000515429.1. In some embodiments, the ASO targets a ACAP1 pre-mRNA sequence comprising a NSAE exon. In some embodiments, the ASO targets a ACAP1 pre-mRNA sequence comprising an intron flanking the 3′ splice site of the NSAE exon. In some embodiments, the ASO targets a ACAP1 pre-mRNA sequence comprising an intron flanking the 5′ splice site of the NSAE exon. In some embodiments, the transcript is selected from the group consisting of transcript_ENST00000308982.12, transcript_ENST00000505192.5, tran-script_ENST00000505602.1, transcript_ENST00000505867.5, transcript_ENST00000508971.1, transcript_ENST00000511227.5, transcript_ENST00000511325.1, tran-script_ENST00000511526.5, transcript_ENST00000512801.5, transcript_ENST00000514336.1, transcript_ENST00000514643.5, and transcript_ENST00000515429.1.

In some embodiments, the ACAP1 pre-mRNA transcript is encoded by a genetic sequence with at least about 80%, 85%, 90%, 95%, 96%, 97%, 98%, 99% or 100% sequence identity to the Ensembl reference number ENSG00000072818.12 or ENSG00000288169.1 or a complement thereof. In some embodiments, the ACAP1 pre-mRNA transcript comprises a sequence with at least about 80%, 85%, 90%, 95%, 96%, 97%, 98%, 99% or 100% sequence identity to a ACAP1 pre-mRNA transcript or a complement thereof described herein.

In some embodiments, the targeted portion of the ACAP1 pre-mRNA comprises a sequence with at least 80%, 85%, 90%, 95%, 97%, or 100% sequence identity to a region comprising at least 8 contiguous nucleic acids of a sequence of premrna_ENST00000308982.12, premrna_ENST00000505192.5, premr-na_ENST00000505602.1, premrna_ENST00000505867.5, premrna_ENST00000508971.1, premrna_ENST00000511227.5, premrna_ENST00000511325.1, premrna_ENST00000511526.5, premrna_ENST00000512801.5, premrna_ENST00000514336.1, premrna_ENST00000514643.5, premrna_ENST00000515429.1, or a sequence of Table 3 or complements thereof. In some embodiments, the targeted portion of the ACAP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 208-SEQ ID NO: 211 or complements thereof. In some embodiments, the targeted portion of the ACAP1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ACOX2 genomic sequence. In some embodiments, the ASO targets a NSAE pre-mRNA transcript from a ACOX2 genomic sequence comprising a NSAE exon. In some embodiments, the ASO targets a NSAE pre-mRNA transcript from a ACOX2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000302819.10, premrna_ENST00000459701.6, premrna_ENST00000459888.1, premrna_ENST00000460921.1, premrna_ENST00000466689.1, premrna_ENST00000466810.5, premrna_ENST00000467738.1, premrna_ENST00000474098.1, premrna_ENST00000475143.5, premrna_ENST00000480791.1, premrna_ENST00000481527.5, premrna_ENST00000489472.1, and premrna_ENST00000492530.1. In some embodiments, the ASO targets a ACOX2 pre-mRNA sequence comprising a NSAE exon. In some embodiments, the ASO targets a ACOX2 pre-mRNA sequence comprising an intron flanking the 3′ splice site of the NSAE exon. In some embodiments, the ASO targets a ACOX2 pre-mRNA sequence comprising an intron flanking the 5′ splice site of the NSAE exon. In some embodiments, the transcript is selected from the group consisting of transcript_ENST00000302819.10, transcript_ENST00000459701.6, transcript_ENST00000459888.1, transcript_ENST00000460921.1, transcript_ENST00000466689.1, transcript_ENST00000466810.5, transcript_ENST00000467738.1, transcript_ENST00000474098.1, transcript_ENST00000475143.5, transcript_ENST00000480791.1, transcript_ENST00000481527.5, transcript_ENST00000489472.1, and transcript_ENST00000492530.1.

In some embodiments, the ACOX2 pre-mRNA transcript is encoded by a genetic sequence with at least about 80%, 85%, 90%, 95%, 96%, 97%, 98%, 99% or 100% sequence identity to the Ensembl reference number ENSG00000168306.13 or a complement thereof. In some embodiments, the ACOX2 pre-mRNA transcript comprises a sequence with at least about 80%, 85%, 90%, 95%, 96%, 97%, 98%, 99% or 100% sequence identity to a ACOX2 pre-mRNA transcript or a complement thereof described herein.

In some embodiments, the targeted portion of the ACOX2 pre-mRNA comprises a sequence with at least 80%, 85%, 90%, 95%, 97%, or 100% sequence identity to a region comprising at least 8 contiguous nucleic acids of a sequence of premrna_ENST00000302819.10, premrna_ENST00000459701.6, premrna_ENST00000459888.1, premrna_ENST00000460921.1, premrna_ENST00000466689.1, premrna_ENST00000466810.5, premrna_ENST00000467738.1, premrna_ENST00000474098.1, premrna_ENST00000475143.5, premrna_ENST00000480791.1, premrna_ENST00000481527.5, premrna_ENST00000489472.1, premrna_ENST00000492530.1 or a sequence of Table 3 or complements thereof. In some embodiments, the targeted portion of the ACOX2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 212-SEQ ID NO: 215 or complements thereof. In some embodiments, the targeted portion of the ACOX2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ACSF3 genomic sequence. In some embodiments, the ASO targets a NSAE pre-mRNA transcript from a ACSF3 genomic sequence comprising a NSAE exon. In some embodiments, the ASO targets a NSAE pre-mRNA transcript from a ACSF3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000317447.9, premrna_ENST00000378345.8, premrna_ENST00000393145.5, premrna_ENST00000406948.7, premrna_ENST00000535176.1, premrna_ENST00000537116.5, premrna_ENST00000537155.1, premrna_ENST00000537290.5, premrna_ENST00000537895.5, premrna_ENST00000538340.5, premrna_ENST00000540697.5, premrna_ENST00000541755.2, premrna_ENST00000542688.5, premrna_ENST00000543676.1, premrna_ENST00000544543.5, premrna_ENST00000562204.1, premrna_ENST00000562750.1, premma_ENST00000614302.5, and premrna_ENST00000649953.1. In some embodiments, the ASO targets a ACSF3 pre-mRNA sequence comprising a NSAE exon. In some embodiments, the ASO targets a ACSF3 pre-mRNA sequence comprising an intron flanking the 3′ splice site of the NSAE exon. In some embodiments, the ASO targets a ACSF3 pre-mRNA sequence comprising an intron flanking the 5′ splice site of the NSAE exon. In some embodiments, the transcript is selected from the group consisting of transcript_ENST00000317447.9, transcript_ENST00000378345.8, transcript_ENST00000393145.5, transcript_ENST00000406948.7, transcript_ENST00000535176.1, transcript_ENST00000537116.5, transcript_ENST00000537155.1, transcript_ENST00000537290.5, transcript_ENST00000537895.5, transcript_ENST00000538340.5, transcript_ENST00000540697.5, transcript_ENST00000541755.2, transcript_ENST00000542688.5, transcript_ENST00000543676.1, transcript_ENST00000544543.5, transcript_ENST00000562204.1, transcript_ENST00000562750.1, transcript_ENST00000614302.5, and transcript_ENST00000649953.1.

In some embodiments, the ACSF3 pre-mRNA transcript is encoded by a genetic sequence with at least about 80%, 85%, 90%, 95%, 96%, 97%, 98%, 99% or 100% sequence identity to the Ensembl reference number ENSG00000176715.17 or a complement thereof. In some embodiments, the ACSF3 pre-mRNA transcript comprises a sequence with at least about 80%, 85%, 90%, 95%, 96%, 97%, 98%, 99% or 100% sequence identity to a ACSF3 pre-mRNA transcript or a complement thereof described herein.

In some embodiments, the targeted portion of the ACSF3 pre-mRNA comprises a sequence with at least 80%, 85%, 90%, 95%, 97%, or 100% sequence identity to a region comprising at least 8 contiguous nucleic acids of a sequence of premrna_ENST00000317447.9, premrna_ENST00000378345.8, premrna_ENST00000393145.5, premrna_ENST00000406948.7, premrna_ENST00000535176.1, premrna_ENST00000537116.5, premrna_ENST00000537155.1, premrna_ENST00000537290.5, premrna_ENST00000537895.5, premrna_ENST00000538340.5, premrna_ENST00000540697.5, premrna_ENST00000541755.2, premrna_ENST00000542688.5, premrna_ENST00000543676.1, premrna_ENST00000544543.5, premrna_ENST00000562204.1, premrna_ENST00000562750.1, premrna_ENST00000614302.5, premrna_ENST00000649953.1 or a sequence of Table 3 or complements thereof. In some embodiments, the targeted portion of the ACSF3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 216-SEQ ID NO: 221 or complements thereof. In some embodiments, the targeted portion of the ACSF3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ACTN4 genomic sequence. In some embodiments, the ASO targets a NSAE pre-mRNA transcript from a ACTN4 genomic sequence comprising a NSAE exon. In some embodiments, the ASO targets a NSAE pre-mRNA transcript from a ACTN4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000252699.7, premrna_ENST00000390009.7, premrna_ENST00000424234.6, premrna_ENST00000440400.1, premrna_ENST00000477174.1, premrna_ENST00000489451.1, premrna_ENST00000495553.1, premrna_ENST00000497637.5, premrna_ENST00000586538.1, premrna_ENST00000588618.5, and premrna_ENST00000589528.1. In some embodiments, the ASO targets a ACTN4 pre-mRNA sequence comprising a NSAE exon. In some embodiments, the ASO targets a ACTN4 pre-mRNA sequence comprising an intron flanking the 3′ splice site of the NSAE exon. In some embodiments, the ASO targets a ACTN4 pre-mRNA sequence comprising an intron flanking the 5′ splice site of the NSAE exon. In some embodiments, the transcript is selected from the group consisting of transcript_ENST00000252699.7, transcript_ENST00000390009.7, transcript_ENST00000424234.6, transcript_ENST00000440400.1, transcript_ENST00000477174.1, transcript_ENST00000489451.1, transcript_ENST00000495553.1, transcript_ENST00000497637.5, transcript_ENST00000586538.1, transcript_ENST00000588618.5, and transcript_ENST00000589528.1.

In some embodiments, the ACTN4 pre-mRNA transcript is encoded by a genetic sequence with at least about 80%, 85%, 90%, 95%, 96%, 97%, 98%, 99% or 100% sequence identity to the Ensembl reference number ENSG00000130402.12 or ENSG00000282844.2 or a complement thereof. In some embodiments, the ACTN4 pre-mRNA transcript comprises a sequence with at least about 80%, 85%, 90%, 95%, 96%, 97%, 98%, 99% or 100% sequence identity to a ACTN4 pre-mRNA transcript or a complement thereof described herein.

In some embodiments, the targeted portion of the ACTN4 pre-mRNA comprises a sequence with at least 80%, 85%, 90%, 95%, 97%, or 100% sequence identity to a region comprising at least 8 contiguous nucleic acids of a sequence of premrna_ENST00000252699.7, premrna_ENST00000390009.7, premrna_ENST00000424234.6, premrna_ENST00000440400.1, premrna_ENST00000477174.1, premrna_ENST00000489451.1, premrna_ENST00000495553.1, premrna_ENST00000497637.5, premrna_ENST00000586538.1, premrna_ENST00000588618.5, premrna_ENST00000589528.1 or a sequence of Table 3 or complements thereof. In some embodiments, the targeted portion of the ACTN4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 222-SEQ ID NO: 225 or complements thereof. In some embodiments, the targeted portion of the ACTN4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ADAM17 genomic sequence. In some embodiments, the ASO targets a NSAE pre-mRNA transcript from a ADAM17 genomic sequence comprising a NSAE exon. In some embodiments, the ASO targets a NSAE pre-mRNA transcript from a ADAM17 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000310823.8, premrna_ENST00000478059.1, premrna_ENST00000618923.2, premrna_ENST00000647610.1, premrna_ENST00000647622.1, premrna_ENST00000647979.1, premrna_ENST00000648002.1, premrna_ENST00000648548.1, premrna_ENST00000648857.1, premrna_ENST00000649068.1, premrna_ENST00000649227.1, premrna_ENST00000649686.1, premrna_ENST00000649798.1, premrna_ENST00000649972.1, premrna_ENST00000650116.1, and premrna_ENST00000650241.1 In some embodiments, the ASO targets a ADAM17 pre-mRNA sequence comprising a NSAE exon. In some embodiments, the ASO targets a ADAM17 pre-mRNA sequence comprising an intron flanking the 3′ splice site of the NSAE exon. In some embodiments, the ASO targets a ADAM17 pre-mRNA sequence comprising an intron flanking the 5′ splice site of the NSAE exon. In some embodiments, the transcript is selected from the group consisting of transcript_ENST00000310823.8, transcript_ENST00000478059.1, transcript_ENST00000618923.2, transcript_ENST00000647610.1, transcript_ENST00000647622.1, transcript_ENST00000647979.1, transcript_ENST00000648002.1, transcript_ENST00000648548.1, transcript_ENST00000648857.1, transcript_ENST00000649068.1, transcript_ENST00000649227.1, transcript_ENST00000649686.1, transcript_ENST00000649798.1, transcript_ENST00000649972.1, transcript_ENST00000650116.1, and transcript_ENST00000650241.1.

In some embodiments, the ADAM17 pre-mRNA transcript is encoded by a genetic sequence with at least about 80%, 85%, 90%, 95%, 96%, 97%, 98%, 99% or 100% sequence identity to the Ensembl reference number ENSG00000151694.14 or a complement thereof. In some embodiments, the ADAM17 pre-mRNA transcript comprises a sequence with at least about 80%, 85%, 90%, 95%, 96%, 97%, 98%, 99% or 100% sequence identity to a ADAM17 pre-mRNA transcript or a complement thereof described herein.

In some embodiments, the targeted portion of the ADAM17 pre-mRNA comprises a sequence with at least 80%, 85%, 90%, 95%, 97%, or 100% sequence identity to a region comprising at least 8 contiguous nucleic acids of a sequence of premrna_ENST00000310823.8, premrna_ENST00000478059.1, premrna_ENST00000618923.2, premrna_ENST00000647610.1, premrna_ENST00000647622.1, premrna_ENST00000647979.1, premrna_ENST00000648002.1, premrna_ENST00000648548.1, premrna_ENST00000648857.1, premrna_ENST00000649068.1, premrna_ENST00000649227.1, premrna_ENST00000649686.1, premrna_ENST00000649798.1, premrna_ENST00000649972.1, premrna_ENST00000650116.1, premrna_ENST00000650241.1 or a sequence of Table 3 or complements thereof. In some embodiments, the targeted portion of the ADAM17 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 226-SEQ ID NO: 229 or complements thereof. In some embodiments, the targeted portion of the ADAM17 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ADAMTS13 genomic sequence. In some embodiments, the ASO targets a NSAE pre-mRNA transcript from a ADAMTS13 genomic sequence comprising a NSAE exon. In some embodiments, the ASO targets a NSAE pre-mRNA transcript from a ADAMTS13 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000355699.6, premrna_ENST00000356589.6, premrna_ENST00000371910.1, premrna_ENST00000371911.7, premrna_ENST00000371916.5, premrna_ENST00000371929.7, premrna_ENST00000474918.1, premrna_ENST00000485925.5, and premrna_ENST00000495234.5. In some embodiments, the ASO targets a ADAMTS13 pre-mRNA sequence comprising a NSAE exon. In some embodiments, the ASO targets a ADAMTS13 pre-mRNA sequence comprising an intron flanking the 3′ splice site of the NSAE exon. In some embodiments, the ASO targets a ADAMTS13 pre-mRNA sequence comprising an intron flanking the 5′ splice site of the NSAE exon. In some embodiments, the transcript is selected from the group consisting of transcript_ENST00000355699.6, transcript_ENST00000356589.6, transcript_ENST00000371910.1, transcript_ENST00000371911.7, transcript_ENST00000371916.5, transcript_ENST00000371929.7, transcript_ENST00000474918.1, transcript_ENST00000485925.5, and transcript_ENST00000495234.5.

In some embodiments, the ADAMTS13 pre-mRNA transcript is encoded by a genetic sequence with at least about 80%, 85%, 90%, 95%, 96%, 97%, 98%, 99% or 100% sequence identity to the Ensembl reference number ENSG00000160323.18 or ENSG00000281244.2 or a complement thereof. In some embodiments, the ADAMTS13 pre-mRNA transcript comprises a sequence with at least about 80%, 85%, 90%, 95%, 96%, 97%, 98%, 99% or 100% sequence identity to a ADAMTS13 pre-mRNA transcript or a complement thereof described herein.

In some embodiments, the targeted portion of the ADAMTS13 pre-mRNA comprises a sequence with at least 80%, 85%, 90%, 95%, 97%, or 100% sequence identity to a region comprising at least 8 contiguous nucleic acids of a sequence of premrna_ENST00000355699.6, premrna_ENST00000356589.6, premrna_ENST00000371910.1, premrna_ENST00000371911.7, premrna_ENST00000371916.5, premrna_ENST00000371929.7, premrna_ENST00000474918.1, premrna_ENST00000485925.5, premrna_ENST00000495234.5, or a sequence of Table 3 or complements thereof. In some embodiments, the targeted portion of the ADAMTS13 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 230-SEQ ID NO: 238 or complements thereof. In some embodiments, the targeted portion of the ADAMTS13 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a AKR1E2 genomic sequence. In some embodiments, the ASO targets a NSAE pre-mRNA transcript from a AKR1E2 genomic sequence comprising a NSAE exon. In some embodiments, the ASO targets a NSAE pre-mRNA transcript from a AKR1E2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000298375.12, premrna_ENST00000334019.4, premrna_ENST00000345253.9, premrna_ENST00000441590.5, premrna_ENST00000462718.7, premrna_ENST00000463345.5, premrna_ENST00000474119.5, premrna_ENST00000487985.1, premrna_ENST00000525281.5, premrna_ENST00000525572.1, premrna_ENST00000525627.1, premrna_ENST00000532248.5, and premrna_ENST00000533295.5. In some embodiments, the ASO targets a AKR1E2 pre-mRNA sequence comprising a NSAE exon. In some embodiments, the ASO targets a AKR1E2 pre-mRNA sequence comprising an intron flanking the 3′ splice site of the NSAE exon. In some embodiments, the ASO targets a AKR1E2 pre-mRNA sequence comprising an intron flanking the 5′ splice site of the NSAE exon. In some embodiments, the transcript is selected from the group consisting of transcript_ENST00000298375.12, transcript_ENST00000334019.4, transcript_ENST00000345253.9, transcript_ENST00000441590.5, transcript_ENST00000462718.7, transcript_ENST00000463345.5, transcript_ENST00000474119.5, transcript_ENST00000487985.1, transcript_ENST00000525281.5, transcript_ENST00000525572.1, transcript_ENST00000525627.1, transcript_ENST00000532248.5, and transcript_ENST00000533295.5.

In some embodiments, the AKR1E2 pre-mRNA transcript is encoded by a genetic sequence with at least about 80%, 85%, 90%, 95%, 96%, 97%, 98%, 99% or 100% sequence identity to the Ensembl reference number ENSG00000165568.18 or a complement thereof. In some embodiments, the AKR1E2 pre-mRNA transcript comprises a sequence with at least about 80%, 85%, 90%, 95%, 96%, 97%, 98%, 99% or 100% sequence identity to a AKR1E2 pre-mRNA transcript or a complement thereof described herein.

In some embodiments, the targeted portion of the AKR1E2 pre-mRNA comprises a sequence with at least 80%, 85%, 90%, 95%, 97%, or 100% sequence identity to a region comprising at least 8 contiguous nucleic acids of a sequence of premrna_ENST00000298375.12, premrna_ENST00000334019.4, premrna_ENST00000345253.9, premrna_ENST00000441590.5, premrna_ENST00000462718.7, premrna_ENST00000463345.5, premrna_ENST00000474119.5, premrna_ENST00000487985.1, premrna_ENST00000525281.5, premrna_ENST00000525572.1, premrna_ENST00000525627.1, premrna_ENST00000532248.5, premrna_ENST00000533295.5 or a sequence of Table 3 or complements thereof. In some embodiments, the targeted portion of the AKR1E2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 239-SEQ ID NO: 242 or complements thereof. In some embodiments, the targeted portion of the AKR1E2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ALAD genomic sequence. In some embodiments, the ASO targets a NSAE pre-mRNA transcript from a ALAD genomic sequence comprising a NSAE exon. In some embodiments, the ASO targets a NSAE pre-mRNA transcript from a ALAD genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000409155.8, premrna_ENST00000445750.1, premrna_ENST00000448137.5, premrna_ENST00000452726.1, premrna_ENST00000464749.5, premrna_ENST00000468504.5, premrna_ENST00000482001.1, premrna_ENST00000482847.5, and premrna_ENST00000494848.1. In some embodiments, the ASO targets a ALAD pre-mRNA sequence comprising a NSAE exon. In some embodiments, the ASO targets a ALAD pre-mRNA sequence comprising an intron flanking the 3′ splice site of the NSAE exon. In some embodiments, the ASO targets a ALAD pre-mRNA sequence comprising an intron flanking the 5′ splice site of the NSAE exon. In some embodiments, the transcript is selected from the group consisting of transcript_ENST00000409155.8, transcript_ENST00000445750.1, transcript_ENST00000448137.5, transcript_ENST00000452726.1, transcript_ENST00000464749.5, transcript_ENST00000468504.5, transcript_ENST00000482001.1, transcript_ENST00000482847.5, and transcript_ENST00000494848.1.

In some embodiments, the ALAD pre-mRNA transcript is encoded by a genetic sequence with at least about 80%, 85%, 90%, 95%, 96%, 97%, 98%, 99% or 100% sequence identity to the Ensembl reference number ENSG00000148218.16 or a complement thereof. In some embodiments, the ALAD pre-mRNA transcript comprises a sequence with at least about 80%, 85%, 90%, 95%, 96%, 97%, 98%, 99% or 100% sequence identity to a ALAD pre-mRNA transcript or a complement thereof described herein.

In some embodiments, the targeted portion of the ALAD pre-mRNA comprises a sequence with at least 80%, 85%, 90%, 95%, 97%, or 100% sequence identity to a region comprising at least 8 contiguous nucleic acids of a sequence of premrna_ENST00000409155.8, premrna_ENST00000445750.1, premrna_ENST00000448137.5, premrna_ENST00000452726.1, premrna_ENST00000464749.5, premrna_ENST00000468504.5, premrna_ENST00000482001.1, premrna_ENST00000482847.5, premrna_ENST00000494848.1 or a sequence of Table 3 or complements thereof. In some embodiments, the targeted portion of the ALAD pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 243-SEQ ID NO: 248 or complements thereof. In some embodiments, the targeted portion of the ALAD pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ALG3 genomic sequence. In some embodiments, the ASO targets a NSAE pre-mRNA transcript from a ALG3 genomic sequence comprising a NSAE exon. In some embodiments, the ASO targets a NSAE pre-mRNA transcript from a ALG3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000397676.8, premrna_ENST00000411922.5, premrna_ENST00000414845.5, premrna_ENST00000423996.5, premrna_ENST00000445626.6, premrna_ENST00000446569.1, premrna_ENST00000455059.5, premrna_ENST00000461415.5, premrna_ENST00000462735.6, premrna_ENST00000463495.5, premrna_ENST00000477959.1, premrna_ENST00000482048.1, premrna_ENST00000485912.1, premrna_ENST00000488976.5. In some embodiments, the ASO targets a ALG3 pre-mRNA sequence comprising a NSAE exon. In some embodiments, the ASO targets a ALG3 pre-mRNA sequence comprising an intron flanking the 3′ splice site of the NSAE exon. In some embodiments, the ASO targets a ALG3 pre-mRNA sequence comprising an intron flanking the 5′ splice site of the NSAE exon. In some embodiments, the transcript is selected from the group consisting of transcript_ENST00000397676.8, transcript_ENST00000411922.5, transcript_ENST00000414845.5, transcript_ENST00000423996.5, transcript_ENST00000445626.6, transcript_ENST00000446569.1, transcript_ENST00000455059.5, transcript_ENST00000461415.5, transcript_ENST00000462735.6, transcript_ENST00000463495.5, transcript_ENST00000477959.1, transcript_ENST00000482048.1, transcript_ENST00000485912.1, and transcript_ENST00000488976.5.

In some embodiments, the ALG3 pre-mRNA transcript is encoded by a genetic sequence with at least about 80%, 85%, 90%, 95%, 96%, 97%, 98%, 99% or 100% sequence identity to the Ensembl reference number ENSG00000214160.10 or a complement thereof. In some embodiments, the ALG3 pre-mRNA transcript comprises a sequence with at least about 80%, 85%, 90%, 95%, 96%, 97%, 98%, 99% or 100% sequence identity to a ALG3 pre-mRNA transcript or a complement thereof described herein.

In some embodiments, the targeted portion of the ALG3 pre-mRNA comprises a sequence with at least 80%, 85%, 90%, 95%, 97%, or 100% sequence identity to a region comprising at least 8 contiguous nucleic acids of a sequence of premrna_ENST00000397676.8, premrna_ENST00000411922.5, premr-na_ENST00000414845.5, premrna_ENST00000423996.5, premrna_ENST00000445626.6, premrna_ENST00000446569.1, premrna_ENST00000455059.5, premrna_ENST00000461415.5, premrna_ENST00000462735.6, premrna_ENST00000463495.5, premrna_ENST00000477959.1, premrna_ENST00000482048.1, premrna_ENST00000485912.1, premrna_ENST00000488976.5, or a sequence of Table 3 or complements thereof. In some embodiments, the targeted portion of the ALG3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 249-SEQ ID NO: 255 or complements thereof. In some embodiments, the targeted portion of the ALG3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ANKRD29 genomic sequence. In some embodiments, the ASO targets a NSAE pre-mRNA transcript from a ANKRD29 genomic sequence comprising a NSAE exon. In some embodiments, the ASO targets a NSAE pre-mRNA transcript from a ANKRD29 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000322980.13, premrna_ENST00000585908.2, premrna_ENST00000586087.1, premrna_ENST00000586511.1, premrna_ENST00000587763.1, premrna_ENST00000591280.5, premrna_ENST00000591617.1, and premrna_ENST00000592179.6. In some embodiments, the ASO targets a ANKRD29 pre-mRNA sequence comprising a NSAE exon. In some embodiments, the ASO targets a ANKRD29 pre-mRNA sequence comprising an intron flanking the 3′ splice site of the NSAE exon. In some embodiments, the ASO targets a ANKRD29 pre-mRNA sequence comprising an intron flanking the 5′ splice site of the NSAE exon. In some embodiments, the transcript is selected from the group consisting of transcript_ENST00000322980.13, transcript_ENST00000585908.2, transcript_ENST00000586087.1, transcript_ENST00000586511.1, transcript_ENST00000587763.1, transcript_ENST00000591280.5, transcript_ENST00000591617.1, and transcript_ENST00000592179.6.

In some embodiments, the ANKRD29 pre-mRNA transcript is encoded by a genetic sequence with at least about 80%, 85%, 90%, 95%, 96%, 97%, 98%, 99% or 100% sequence identity to the Ensembl reference number ENSG00000154065.17 or a complement thereof. In some embodiments, the ANKRD29 pre-mRNA transcript comprises a sequence with at least about 80%, 85%, 90%, 95%, 96%, 97%, 98%, 99% or 100% sequence identity to a ANKRD29 pre-mRNA transcript or a complement thereof described herein.

In some embodiments, the targeted portion of the ANKRD29 pre-mRNA comprises a sequence with at least 80%, 85%, 90%, 95%, 97%, or 100% sequence identity to a region comprising at least 8 contiguous nucleic acids of a sequence of premrna_ENST00000322980.13, premrna_ENST00000585908.2, premrna_ENST00000586087.1, premrna_ENST00000586511.1, premrna_ENST00000587763.1, premrna_ENST00000591280.5, premrna_ENST00000591617.1, premrna_ENST00000592179.6 or a sequence of Table 3 or complements thereof. In some embodiments, the targeted portion of the ANKRD29 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 256-SEQ ID NO: 259 or complements thereof. In some embodiments, the targeted portion of the ANKRD29 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ANKS3 genomic sequence. In some embodiments, the ASO targets a NSAE pre-mRNA transcript from a ANKS3 genomic sequence comprising a NSAE exon. In some embodiments, the ASO targets a NSAE pre-mRNA transcript from a ANKS3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000304283.9, premrna_ENST00000446014.6, premrna_ENST00000450067.6, premrna_ENST00000585773.5, premrna_ENST00000586159.5, premrna_ENST00000586166.5, premrna_ENST00000586605.5, premrna_ENST00000586632.1, premrna_ENST00000587005.5, premrna_ENST00000588398.1, premrna_ENST00000588513.1, premrna_ENST00000589035.5, premrna_ENST00000589065.5, premrna_ENST00000590147.5, premrna_ENST00000590193.5, premrna_ENST00000590689.1, premrna_ENST00000590730.5, premrna_ENST00000590803.5, premrna_ENST00000591185.5, premrna_ENST00000591281.5, premrna_ENST00000591653.5, premrna_ENST00000592068.5, premrna_ENST00000592077.5, premrna_ENST00000592190.1, premrna_ENST00000592421.5, premrna_ENST00000592698.5, premrna_ENST00000592711.5, premrna_ENST00000592840.1, premrna_ENST00000593120.5, and premrna_ENST00000614075.4. In some embodiments, the ASO targets a ANKS3 pre-mRNA sequence comprising a NSAE exon. In some embodiments, the ASO targets a ANKS3 pre-mRNA sequence comprising an intron flanking the 3′ splice site of the NSAE exon. In some embodiments, the ASO targets a ANKS3 pre-mRNA sequence comprising an intron flanking the 5′ splice site of the NSAE exon. In some embodiments, the transcript is selected from the group consisting of transcript_ENST00000304283.9, transcript_ENST00000446014.6, transcript_ENST00000450067.6, transcript_ENST00000585773.5, transcript_ENST00000586159.5, transcript_ENST00000586166.5, transcript_ENST00000586605.5, transcript_ENST00000586632.1, transcript_ENST00000587005.5, transcript_ENST00000588398.1, transcript_ENST00000588513.1, transcript_ENST00000589035.5, transcript_ENST00000589065.5, transcript_ENST00000590147.5, transcript_ENST00000590193.5, transcript_ENST00000590689.1, transcript_ENST00000590730.5, transcript_ENST00000590803.5, transcript_ENST00000591185.5, transcript_ENST00000591281.5, transcript_ENST00000591653.5, transcript_ENST00000592068.5, transcript_ENST00000592077.5, transcript_ENST00000592190.1, transcript_ENST00000592421.5, transcript_ENST00000592698.5, transcript_ENST00000592711.5, transcript_ENST00000592840.1, transcript_ENST00000593120.5, and transcript_ENST00000614075.4.

In some embodiments, the ANKS3 pre-mRNA transcript is encoded by a genetic sequence with at least about 80%, 85%, 90%, 95%, 96%, 97%, 98%, 99% or 100% sequence identity to the Ensembl reference number ENSG00000168096.15 or a complement thereof. In some embodiments, the ANKS3 pre-mRNA transcript comprises a sequence with at least about 80%, 85%, 90%, 95%, 96%, 97%, 98%, 99% or 100% sequence identity to a ANKS3 pre-mRNA transcript or a complement thereof described herein.

In some embodiments, the targeted portion of the ANKS3 pre-mRNA comprises a sequence with at least 80%, 85%, 90%, 95%, 97%, or 100% sequence identity to a region comprising at least 8 contiguous nucleic acids of a sequence of premrna_ENST00000304283.9, premrna_ENST00000446014.6, premrna_ENST00000450067.6, premrna_ENST00000585773.5, premrna_ENST00000586159.5, premrna_ENST00000586166.5, premrna_ENST00000586605.5, premrna_ENST00000586632.1, premrna_ENST00000587005.5, premrna_ENST00000588398.1, premrna_ENST00000588513.1, premrna_ENST00000589035.5, premrna_ENST00000589065.5, premrna_ENST00000590147.5, premrna_ENST00000590193.5, premrna_ENST00000590689.1, premrna_ENST00000590730.5, premrna_ENST00000590803.5, premrna_ENST00000591185.5, premrna_ENST00000591281.5, premrna_ENST00000591653.5, premrna_ENST00000592068.5, premrna_ENST00000592077.5, premrna_ENST00000592190.1, premrna_ENST00000592421.5, premrna_ENST00000592698.5, premrna_ENST00000592711.5, premrna_ENST00000592840.1, premrna_ENST00000593120.5, premrna_ENST00000614075.4, or a sequence of Table 3 or complements thereof. In some embodiments, the targeted portion of the ANKS3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 260-SEQ ID NO: 263 or complements thereof. In some embodiments, the targeted portion of the ANKS3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ANO4 genomic sequence. In some embodiments, the ASO targets a NSAE pre-mRNA transcript from a ANO4 genomic sequence comprising a NSAE exon. In some embodiments, the ASO targets a NSAE pre-mRNA transcript from a ANO4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000392977.8, premrna_ENST00000392979.7, premrna_ENST00000546991.1, premrna_ENST00000548940.1, premrna_ENST00000549155.6, premrna_ENST00000549234.1, premrna_ENST00000550015.1, premrna_ENST00000551148.1, and premrna_ENST00000644049.1. In some embodiments, the ASO targets a ANO4 pre-mRNA sequence comprising a NSAE exon. In some embodiments, the ASO targets a ANO4 pre-mRNA sequence comprising an intron flanking the 3′ splice site of the NSAE exon. In some embodiments, the ASO targets a ANO4 pre-mRNA sequence comprising an intron flanking the 5′ splice site of the NSAE exon. In some embodiments, the transcript is selected from the group consisting of transcript_ENST00000392977.8, transcript_ENST00000392979.7, transcript_ENST00000546991.1, transcript_ENST00000548940.1, transcript_ENST00000549155.6, transcript_ENST00000549234.1, transcript_ENST00000550015.1, transcript_ENST00000551148.1, and transcript_ENST00000644049.1.

In some embodiments, the ANO4 pre-mRNA transcript is encoded by a genetic sequence with at least about 80%, 85%, 90%, 95%, 96%, 97%, 98%, 99% or 100% sequence identity to the Ensembl reference number ENSG00000151572.18 or ENSG00000262139.9 or a complement thereof. In some embodiments, the ANO4 pre-mRNA transcript comprises a sequence with at least about 80%, 85%, 90%, 95%, 96%, 97%, 98%, 99% or 100% sequence identity to a ANO4 pre-mRNA transcript or a complement thereof described herein.

In some embodiments, the targeted portion of the ANO4 pre-mRNA comprises a sequence with at least 80%, 85%, 90%, 95%, 97%, or 100% sequence identity to a region comprising at least 8 contiguous nucleic acids of a sequence of premrna_ENST00000392977.8, premrna_ENST00000392979.7, premrna_ENST00000546991.1, premrna_ENST00000548940.1, premrna_ENST00000549155.6, premrna_ENST00000549234.1, premrna_ENST00000550015.1, premrna_ENST00000551148.1, premrna_ENST00000644049.1 or a sequence of Table 3 or complements thereof. In some embodiments, the targeted portion of the ANO4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 264-SEQ ID NO: 267 or complements thereof. In some embodiments, the targeted portion of the ANO4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a AP3B2 genomic sequence. In some embodiments, the ASO targets a NSAE pre-mRNA transcript from a AP3B2 genomic sequence comprising a NSAE exon. In some embodiments, the ASO targets a NSAE pre-mRNA transcript from a AP3B2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000261722.8, premrna_ENST00000535348.5, premrna_ENST00000535359.6, premrna_ENST00000535385.6, premrna_ENST00000535513.2, premrna_ENST00000537735.2, premrna_ENST00000541693.5, premrna_ENST00000542200.2, premrna_ENST00000543938.6, premrna_ENST00000559888.1, premrna_ENST00000560529.1, premrna_ENST00000561455.5, premrna_ENST00000620652.4, premrna_ENST00000642989.2, premrna_ENST00000652847.1, premrna_ENST00000657321.1, premrna_ENST00000659252.1, premrna_ENST00000660624.1, premrna_ENST00000661532.1, premrna_ENST00000663651.1, premrna_ENST00000664460.1, premrna_ENST00000665513.1, premrna_ENST00000666055.1, premrna_ENST00000666672.1, premrna_ENST00000666894.1, premrna_ENST00000666973.1, premrna_ENST00000667758.1, premrna_ENST00000668385.1, premrna_ENST00000668458.1, premrna_ENST00000668990.2, premrna_ENST00000669880.1, and premrna_ENST00000669930.1. In some embodiments, the ASO targets a AP3B2 pre-mRNA sequence comprising a NSAE exon. In some embodiments, the ASO targets a AP3B2 pre-mRNA sequence comprising an intron flanking the 3′ splice site of the NSAE exon. In some embodiments, the ASO targets a AP3B2 pre-mRNA sequence comprising an intron flanking the 5′ splice site of the NSAE exon. In some embodiments, the transcript is selected from the group consisting of transcript_ENST00000261722.8, transcript_ENST00000535348.5, transcript_ENST00000535359.6, transcript_ENST00000535385.6, transcript_ENST00000535513.2, transcript_ENST00000537735.2, transcript_ENST00000541693.5, transcript_ENST00000542200.2, transcript_ENST00000543938.6, transcript_ENST00000559888.1, transcript_ENST00000560529.1, transcript_ENST00000561455.5, transcript_ENST00000620652.4, transcript_ENST00000642989.2, transcript_ENST00000652847.1, transcript_ENST00000657321.1, transcript_ENST00000659252.1, transcript_ENST00000660624.1, transcript_ENST00000661532.1, transcript_ENST00000663651.1, transcript_ENST00000664460.1, transcript_ENST00000665513.1, transcript_ENST00000666055.1, transcript_ENST00000666672.1, transcript_ENST00000666894.1, transcript_ENST00000666973.1, transcript_ENST00000667758.1, transcript_ENST00000668385.1, transcript_ENST00000668458.1, transcript_ENST00000668990.2, transcript_ENST00000669880.1, and transcript_ENST00000669930.1.

In some embodiments, the AP3B2 pre-mRNA transcript is encoded by a genetic sequence with at least about 80%, 85%, 90%, 95%, 96%, 97%, 98%, 99% or 100% sequence identity to the Ensembl reference number ENSG00000103723.15 or a complement thereof. In some embodiments, the AP3B2 pre-mRNA transcript comprises a sequence with at least about 80%, 85%, 90%, 95%, 96%, 97%, 98%, 99% or 100% sequence identity to a AP3B2 pre-mRNA transcript or a complement thereof described herein.

In some embodiments, the targeted portion of the AP3B2 pre-mRNA comprises a sequence with at least 80%, 85%, 90%, 95%, 97%, or 100% sequence identity to a region comprising at least 8 contiguous nucleic acids of a sequence of premrna_ENST00000261722.8, premrna_ENST00000535348.5, premrna_ENST00000535359.6, premrna_ENST00000535385.6, premrna_ENST00000535513.2, premrna_ENST00000537735.2, premrna_ENST00000541693.5, premrna_ENST00000542200.2, premrna_ENST00000543938.6, premrna_ENST00000559888.1, premrna_ENST00000560529.1, premrna_ENST00000561455.5, premrna_ENST00000620652.4, premrna_ENST00000642989.2, premrna_ENST00000652847.1, premrna_ENST00000657321.1, premrna_ENST00000659252.1, premrna_ENST00000660624.1, premrna_ENST00000661532.1, premrna_ENST00000663651.1, premrna_ENST00000664460.1, premrna_ENST00000665513.1, premrna_ENST00000666055.1, premrna_ENST00000666672.1, premrna_ENST00000666894.1, premrna_ENST00000666973.1, premrna_ENST00000667758.1, premrna_ENST00000668385.1, premrna_ENST00000668458.1, premrna_ENST00000668990.2, premrna_ENST00000669880.1, premrna_ENST00000669930.1 or a sequence of Table 3 or complements thereof. In some embodiments, the targeted portion of the AP3B2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 268-SEQ ID NO: 271 or complements thereof. In some embodiments, the targeted portion of the AP3B2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a AP3M1 genomic sequence. In some embodiments, the ASO targets a NSAE pre-mRNA transcript from a AP3M1 genomic sequence comprising a NSAE exon. In some embodiments, the ASO targets a NSAE pre-mRNA transcript from a AP3M1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000355264.8, premrna_ENST00000372745.1, premrna_ENST00000480373.1, and premrna_ENST00000487653.1. In some embodiments, the ASO targets aAP3M1 pre-mRNA sequence comprising a NSAE exon. In some embodiments, the ASO targets a AP3M1 pre-mRNA sequence comprising an intron flanking the 3′ splice site of the NSAE exon. In some embodiments, the ASO targets a AP3M1 pre-mRNA sequence comprising an intron flanking the 5′ splice site of the NSAE exon. In some embodiments, the transcript is selected from the group consisting of transcript_ENST00000355264.8, transcript_ENST00000372745.1, transcript_ENST00000480373.1, and transcript_ENST00000487653.1.

In some embodiments, the AP3M1 pre-mRNA transcript is encoded by a genetic sequence with at least about 80%, 85%, 90%, 95%, 96%, 97%, 98%, 99% or 100% sequence identity to the Ensembl reference number ENSG00000185009.12 or a complement thereof. In some embodiments, the AP3M1 pre-mRNA transcript comprises a sequence with at least about 80%, 85%, 90%, 95%, 96%, 97%, 98%, 99% or 100% sequence identity to a AP3M1 pre-mRNA transcript or a complement thereof described herein.

In some embodiments, the targeted portion of the AP3M1 pre-mRNA comprises a sequence with at least 80%, 85%, 90%, 95%, 97%, or 100% sequence identity to a region comprising at least 8 contiguous nucleic acids of a sequence of premrna_ENST00000355264.8, premrna_ENST00000372745.1, premrna_ENST00000480373.1, premrna_ENST00000487653.1 or a sequence of Table 3 or complements thereof. In some embodiments, the targeted portion of the AP3M1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 272-SEQ ID NO: 275 or complements thereof. In some embodiments, the targeted portion of the AP3M1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a AP5Z1 genomic sequence. In some embodiments, the ASO targets a NSAE pre-mRNA transcript from a AP5Z1 genomic sequence comprising a NSAE exon. In some embodiments, the ASO targets a NSAE pre-mRNA transcript from a AP5Z1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000469614.1, premrna_ENST00000477454.1, premrna_ENST00000477680.6, premrna_ENST00000490487.1, premrna_ENST00000491375.1, premrna_ENST00000496303.6, premrna_ENST00000647628.1, premrna_ENST00000647984.1, premrna_ENST00000648237.1, premrna_ENST00000648360.1, premrna_ENST00000648765.1, premrna_ENST00000648925.1, premrna_ENST00000649063.2, premrna_ENST00000649315.1, premrna_ENST00000649419.1, premrna_ENST00000649736.1, premrna_ENST00000650310.1, premrna_ENST00000650451.1, and premrna_ENST00000650581.1. In some embodiments, the ASO targets a AP5Z1 pre-mRNA sequence comprising a NSAE exon. In some embodiments, the ASO targets a AP5Z1 pre-mRNA sequence comprising an intron flanking the 3′ splice site of the NSAE exon. In some embodiments, the ASO targets a AP5Z1 pre-mRNA sequence comprising an intron flanking the 5′ splice site of the NSAE exon. In some embodiments, the transcript is selected from the group consisting of transcript_ENST00000469614.1, transcript_ENST00000477454.1, transcript_ENST00000477680.6, transcript_ENST00000490487.1, transcript_ENST00000491375.1, transcript_ENST00000496303.6, transcript_ENST00000647628.1, transcript_ENST00000647984.1, transcript_ENST00000648237.1, transcript_ENST00000648360.1, transcript_ENST00000648765.1, transcript_ENST00000648925.1, transcript_ENST00000649063.2, transcript_ENST00000649315.1, transcript_ENST00000649419.1, transcript_ENST00000649736.1, transcript_ENST00000650310.1, transcript_ENST00000650451.1, and transcript_ENST00000650581.1.

In some embodiments, the AP5Z1 pre-mRNA transcript is encoded by a genetic sequence with at least about 80%, 85%, 90%, 95%, 96%, 97%, 98%, 99% or 100% sequence identity to the Ensembl reference number ENSG00000242802.9 or a complement thereof. In some embodiments, the AP5Z1 pre-mRNA transcript comprises a sequence with at least about 80%, 85%, 90%, 95%, 96%, 97%, 98%, 99% or 100% sequence identity to a AP5Z1 pre-mRNA transcript or a complement thereof described herein.

In some embodiments, the targeted portion of the AP5Z1 pre-mRNA comprises a sequence with at least 80%, 85%, 90%, 95%, 97%, or 100% sequence identity to a region comprising at least 8 contiguous nucleic acids of a sequence of premrna_ENST00000469614.1, premrna_ENST00000477454.1, premrna_ENST00000477680.6, premrna_ENST00000490487.1, premrna_ENST00000491375.1, premrna_ENST00000496303.6, premrna_ENST00000647628.1, premrna_ENST00000647984.1, premrna_ENST00000648237.1, premrna_ENST00000648360.1, premrna_ENST00000648765.1, premrna_ENST00000648925.1, premrna_ENST00000649063.2, premrna_ENST00000649315.1, premrna_ENST00000649419.1, premrna_ENST00000649736.1, premrna_ENST00000650310.1, premrna_ENST00000650451.1, premrna_ENST00000650581.1 or a sequence of Table 3 or complements thereof. In some embodiments, the targeted portion of the AP5Z1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 276-SEQ ID NO: 279 or complements thereof. In some embodiments, the targeted portion of the AP5Z1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ARNTL genomic sequence. In some embodiments, the ASO targets a NSAE pre-mRNA transcript from a ARNTL genomic sequence comprising a NSAE exon. In some embodiments, the ASO targets a NSAE pre-mRNA transcript from a ARNTL genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000389707.8, premrna_ENST00000401424.6, premrna_ENST00000403290.5, premrna_ENST00000403482.7, premrna_ENST00000403510.8, premrna_ENST00000472842.1, premrna_ENST00000480685.5, premrna_ENST00000482049.6, premrna_ENST00000485918.2, premrna_ENST00000497429.5, premrna_ENST00000524392.5, premrna_ENST00000527998.5, premrna_ENST00000529050.5, premrna_ENST00000529388.6, premrna_ENST00000529390.1, premrna_ENST00000529825.6, premrna_ENST00000530357.6, premrna_ENST00000531665.5, premrna_ENST00000533520.5, premrna_ENST00000534102.1, premrna_ENST00000534544.5, premrna_ENST00000673626.1, premrna_ENST00000673817.1, premrna_ENST00000673834.1, premrna_ENST00000673837.1, premrna_ENST00000673868.1, premrna_ENST00000673888.1, premrna_ENST00000673892.1, and premrna_ENST00000674108.1. In some embodiments, the ASO targets a ARNTL pre-mRNA sequence comprising a NSAE exon. In some embodiments, the ASO targets a ARNTL pre-mRNA sequence comprising an intron flanking the 3′ splice site of the NSAE exon. In some embodiments, the ASO targets a ARNTL pre-mRNA sequence comprising an intron flanking the 5′ splice site of the NSAE exon. In some embodiments, the transcript is selected from the group consisting of transcript_ENST00000389707.8, transcript_ENST00000401424.6, transcript_ENST00000403290.5, transcript_ENST00000403482.7, transcript_ENST00000403510.8, transcript_ENST00000472842.1, transcript_ENST00000480685.5, transcript_ENST00000482049.6, transcript_ENST00000485918.2, transcript_ENST00000497429.5, transcript_ENST00000524392.5, transcript_ENST00000527998.5, transcript_ENST00000529050.5, transcript_ENST00000529388.6, transcript_ENST00000529390.1, transcript_ENST00000529825.6, transcript_ENST00000530357.6, transcript_ENST00000531665.5, transcript_ENST00000533520.5, transcript_ENST00000534102.1, transcript_ENST00000534544.5, transcript_ENST00000673626.1, transcript_ENST00000673817.1, transcript_ENST00000673834.1, transcript_ENST00000673837.1, transcript_ENST00000673868.1, transcript_ENST00000673888.1, transcript_ENST00000673892.1, and transcript_ENST00000674108.1.

In some embodiments, the ARNTL pre-mRNA transcript is encoded by a genetic sequence with at least about 80%, 85%, 90%, 95%, 96%, 97%, 98%, 99% or 100% sequence identity to the Ensembl reference number ENSG00000133794.19 or a complement thereof. In some embodiments, the ARNTL pre-mRNA transcript comprises a sequence with at least about 80%, 85%, 90%, 95%, 96%, 97%, 98%, 99% or 100% sequence identity to a ARNTL pre-mRNA transcript or a complement thereof described herein.

In some embodiments, the targeted portion of the ARNTL pre-mRNA comprises a sequence with at least 80%, 85%, 90%, 95%, 97%, or 100% sequence identity to a region comprising at least 8 contiguous nucleic acids of a sequence of premrna_ENST00000389707.8, premrna_ENST00000401424.6, premrna_ENST00000403290.5, premrna_ENST00000403482.7, premrna_ENST00000403510.8, premrna_ENST00000472842.1, premrna_ENST00000480685.5, premrna_ENST00000482049.6, premrna_ENST00000485918.2, premrna_ENST00000497429.5, premrna_ENST00000524392.5, premrna_ENST00000527998.5, premrna_ENST00000529050.5, premrna_ENST00000529388.6, premrna_ENST00000529390.1, premrna_ENST00000529825.6, premrna_ENST00000530357.6, premrna_ENST00000531665.5, premrna_ENST00000533520.5, premrna_ENST00000534102.1, premrna_ENST00000534544.5, premrna_ENST00000673626.1, premrna_ENST00000673817.1, premrna_ENST00000673834.1, premrna_ENST00000673837.1, premrna_ENST00000673868.1, premrna_ENST00000673888.1, premrna_ENST00000673892.1, premrna_ENST00000674108.1, or a sequence of Table 3 or complements thereof. In some embodiments, the targeted portion of the ARNTL pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 280-SEQ ID NO: 283 or complements thereof. In some embodiments, the targeted portion of the ARNTL pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ASAP3 genomic sequence. In some embodiments, the ASO targets a NSAE pre-mRNA transcript from a ASAP3 genomic sequence comprising a NSAE exon. In some embodiments, the ASO targets a NSAE pre-mRNA transcript from a ASAP3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000336689.8, premrna_ENST00000437606.6, premrna_ENST00000449467.2, premrna_ENST00000465372.5, premrna_ENST00000475814.5, premrna_ENST00000478858.5, premrna_ENST00000484418.1, premrna_ENST00000492982.6, premrna_ENST00000495646.5, premrna_ENST00000530874.1, premrna_ENST00000608765.1, and premrna_ENST00000618240.4. In some embodiments, the ASO targets a ASAP3 pre-mRNA sequence comprising a NSAE exon. In some embodiments, the ASO targets a ASAP3 pre-mRNA sequence comprising an intron flanking the 3′ splice site of the NSAE exon. In some embodiments, the ASO targets a ASAP3 pre-mRNA sequence comprising an intron flanking the 5′ splice site of the NSAE exon. In some embodiments, the transcript is selected from the group consisting of transcript_ENST00000336689.8, transcript_ENST00000437606.6, transcript_ENST00000449467.2, transcript_ENST00000465372.5, transcript_ENST00000475814.5, transcript_ENST00000478858.5, transcript_ENST00000484418.1, transcript_ENST00000492982.6, transcript_ENST00000495646.5, transcript_ENST00000530874.1, transcript_ENST00000608765.1, and transcript_ENST00000618240.4.

In some embodiments, the ASAP3 pre-mRNA transcript is encoded by a genetic sequence with at least about 80%, 85%, 90%, 95%, 96%, 97%, 98%, 99% or 100% sequence identity to the Ensembl reference number ENSG00000088280.19 or ENSG00000282854.2 a complement thereof. In some embodiments, the ASAP3 pre-mRNA transcript comprises a sequence with at least about 80%, 85%, 90%, 95%, 96%, 97%, 98%, 99% or 100% sequence identity to a ASAP3 pre-mRNA transcript or a complement thereof described herein.

In some embodiments, the targeted portion of the ASAP3 pre-mRNA comprises a sequence with at least 80%, 85%, 90%, 95%, 97%, or 100% sequence identity to a region comprising at least 8 contiguous nucleic acids of a sequence of premrna_ENST00000336689.8, premrna_ENST00000437606.6, premrna_ENST00000449467.2, premrna_ENST00000465372.5, premrna_ENST00000475814.5, premrna_ENST00000478858.5, premrna_ENST00000484418.1, premrna_ENST00000492982.6, premrna_ENST00000495646.5, premrna_ENST00000530874.1, premrna_ENST00000608765.1, premrna_ENST00000618240.4, or a sequence of Table 3 or complements thereof. In some embodiments, the targeted portion of the ASAP3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 284-SEQ ID NO: 287 or complements thereof. In some embodiments, the targeted portion of the ASAP3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ATRX genomic sequence. In some embodiments, the ASO targets a NSAE pre-mRNA transcript from a ATRX genomic sequence comprising a NSAE exon. In some embodiments, the ASO targets a NSAE pre-mRNA transcript from a ATRX genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000373344.10, premrna_ENST00000395603.7, premrna_ENST00000400866.4, premrna_ENST00000460639.2, premrna_ENST00000479487.1, premrna_ENST00000480283.5, premrna_ENST00000493470.2, premrna_ENST00000622960.1, premrna_ENST00000623242.3, premrna_ENST00000623316.1, premrna_ENST00000623321.3, premrna_ENST00000623706.3, premrna_ENST00000624032.3, premrna_ENST00000624166.3, premrna_ENST00000624193.1, premrna_ENST00000624403.1, premrna_ENST00000624668.3, premrna_ENST00000624766.1, premrna_ENST00000625063.3, premrna_ENST00000635865.1, premrna_ENST00000636152.1, premrna_ENST00000636868.1, premrna_ENST00000637175.1, and premrna_ENST00000637959.1. In some embodiments, the ASO targets a ATRX pre-mRNA sequence comprising a NSAE exon. In some embodiments, the ASO targets a ATRX pre-mRNA sequence comprising an intron flanking the 3′ splice site of the NSAE exon. In some embodiments, the ASO targets a ATRX pre-mRNA sequence comprising an intron flanking the 5′ splice site of the NSAE exon. In some embodiments, the transcript is selected from the group consisting of transcript_ENST00000373344.10, transcript_ENST00000395603.7, transcript_ENST00000400866.4, transcript_ENST00000460639.2, transcript_ENST00000479487.1, transcript_ENST00000480283.5, transcript_ENST00000493470.2, transcript_ENST00000622960.1, transcript_ENST00000623242.3, transcript_ENST00000623316.1, transcript_ENST00000623321.3, transcript_ENST00000623706.3, transcript_ENST00000624032.3, transcript_ENST00000624166.3, transcript_ENST00000624193.1, transcript_ENST00000624403.1, transcript_ENST00000624668.3, transcript_ENST00000624766.1, transcript_ENST00000625063.3, transcript_ENST00000635865.1, transcript_ENST00000636152.1, transcript_ENST00000636868.1, transcript_ENST00000637175.1, and transcript_ENST00000637959.1.

In some embodiments, the ATRX pre-mRNA transcript is encoded by a genetic sequence with at least about 80%, 85%, 90%, 95%, 96%, 97%, 98%, 99% or 100% sequence identity to the Ensembl reference number ENSG00000085224.22 or a complement thereof. In some embodiments, the ATRX pre-mRNA transcript comprises a sequence with at least about 80%, 85%, 90%, 95%, 96%, 97%, 98%, 99% or 100% sequence identity to a ATRX pre-mRNA transcript or a complement thereof described herein.

In some embodiments, the targeted portion of the ATRX pre-mRNA comprises a sequence with at least 80%, 85%, 90%, 95%, 97%, or 100% sequence identity to a region comprising at least 8 contiguous nucleic acids of a sequence of premrna_ENST00000373344.10, premrna_ENST00000395603.7, premrna_ENST00000400866.4, premrna_ENST00000460639.2, premrna_ENST00000479487.1, premrna_ENST00000480283.5, premrna_ENST00000493470.2, premrna_ENST00000622960.1, premrna_ENST00000623242.3, premrna_ENST00000623316.1, premrna_ENST00000623321.3, premrna_ENST00000623706.3, premrna_ENST00000624032.3, premrna_ENST00000624166.3, premrna_ENST00000624193.1, premrna_ENST00000624403.1, premrna_ENST00000624668.3, premrna_ENST00000624766.1, premrna_ENST00000625063.3, premrna_ENST00000635865.1, premrna_ENST00000636152.1, premrna_ENST00000636868.1, premrna_ENST00000637175.1, premrna_ENST00000637959.1 or a sequence of Table 3 or complements thereof. In some embodiments, the targeted portion of the ATRX pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 288-SEQ ID NO: 297 or complements thereof. In some embodiments, the targeted portion of the ATRX pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a BBS2 genomic sequence. In some embodiments, the ASO targets a NSAE pre-mRNA transcript from a BBS2 genomic sequence comprising a NSAE exon. In some embodiments, the ASO targets a NSAE pre-mRNA transcript from a BBS2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000245157.10, premrna_ENST00000561853.1, premrna_ENST00000561877.1, premrna_ENST00000561951.5, premrna_ENST00000562012.1, premrna_ENST00000562059.1, premrna_ENST00000562813.1, premrna_ENST00000564123.6, premrna_ENST00000564459.5, premrna_ENST00000565378.1, premrna_ENST00000565781.5, premrna_ENST00000565859.1, premrna_ENST00000566210.1, premrna_ENST00000566410.1, premrna_ENST00000566452.1, premrna_ENST00000566495.1, premrna_ENST00000566689.5, premrna_ENST00000568104.5, premrna_ENST00000569192.5, premrna_ENST00000569342.5, and premrna_ENST00000569941.5. In some embodiments, the ASO targets a BBS2 pre-mRNA sequence comprising a NSAE exon. In some embodiments, the ASO targets a BBS2 pre-mRNA sequence comprising an intron flanking the 3′ splice site of the NSAE exon. In some embodiments, the ASO targets a BBS2 pre-mRNA sequence comprising an intron flanking the 5′ splice site of the NSAE exon. In some embodiments, the transcript is selected from the group consisting of transcript_ENST00000245157.10, transcript_ENST00000561853.1, transcript_ENST00000561877.1, transcript_ENST00000561951.5, transcript_ENST00000562012.1, transcript_ENST00000562059.1, transcript_ENST00000562813.1, transcript_ENST00000564123.6, transcript_ENST00000564459.5, transcript_ENST00000565378.1, transcript_ENST00000565781.5, transcript_ENST00000565859.1, transcript_ENST00000566210.1, transcript_ENST00000566410.1, transcript_ENST00000566452.1, transcript_ENST00000566495.1, transcript_ENST00000566689.5, transcript_ENST00000568104.5, transcript_ENST00000569192.5, transcript_ENST00000569342.5, and transcript_ENST00000569941.5.

In some embodiments, the BBS2 pre-mRNA transcript is encoded by a genetic sequence with at least about 80%, 85%, 90%, 95%, 96%, 97%, 98%, 99% or 100% sequence identity to the Ensembl reference number ENSG00000125124.12 or a complement thereof. In some embodiments, the BBS2 pre-mRNA transcript comprises a sequence with at least about 80%, 85%, 90%, 95%, 96%, 97%, 98%, 99% or 100% sequence identity to a BBS2 pre-mRNA transcript or a complement thereof described herein.

In some embodiments, the targeted portion of the BBS2 pre-mRNA comprises a sequence with at least 80%, 85%, 90%, 95%, 97%, or 100% sequence identity to a region comprising at least 8 contiguous nucleic acids of a sequence of premrna_ENST00000245157.10, premrna_ENST00000561853.1, premrna_ENST00000561877.1, premrna_ENST00000561951.5, premrna_ENST00000562012.1, premrna_ENST00000562059.1, premrna_ENST00000562813.1, premrna_ENST00000564123.6, premrna_ENST00000564459.5, premrna_ENST00000565378.1, premrna_ENST00000565781.5, premrna_ENST00000565859.1, premrna_ENST00000566210.1, premrna_ENST00000566410.1, premrna_ENST00000566452.1, premrna_ENST00000566495.1, premrna_ENST00000566689.5, premrna_ENST00000568104.5, premrna_ENST00000569192.5, premrna_ENST00000569342.5, premrna_ENST00000569941.5 or a sequence of Table 3 or complements thereof. In some embodiments, the targeted portion of the BBS2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 298-SEQ ID NO: 301 or complements thereof. In some embodiments, the targeted portion of the BBS2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a BBS4 genomic sequence. In some embodiments, the ASO targets a NSAE pre-mRNA transcript from a BBS4 genomic sequence comprising a NSAE exon. In some embodiments, the ASO targets a NSAE pre-mRNA transcript from a BBS4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000268057.9, premrna_ENST00000395205.6, premrna_ENST00000561914.5, premrna_ENST00000562084.5, premrna_ENST00000562219.1, premrna_ENST00000563600.5, premrna_ENST00000564239.1, premrna_ENST00000565160.5, premrna_ENST00000566197.1, premrna_ENST00000566400.5, premrna_ENST00000566829.1, premrna_ENST00000566938.5, premrna_ENST00000567279.5, premrna_ENST00000568535.1, premrna_ENST00000569001.1, premrna_ENST00000569151.1, premrna_ENST00000569338.5, and premrna_ENST00000569440.5. In some embodiments, the ASO targets a BBS4 pre-mRNA sequence comprising a NSAE exon. In some embodiments, the ASO targets a BBS4 pre-mRNA sequence comprising an intron flanking the 3′ splice site of the NSAE exon. In some embodiments, the ASO targets a BBS4 pre-mRNA sequence comprising an intron flanking the 5′ splice site of the NSAE exon. In some embodiments, the transcript is selected from the group consisting of transcript_ENST00000268057.9, transcript_ENST00000395205.6, transcript_ENST00000561914.5, transcript_ENST00000562084.5, transcript_ENST00000562219.1, transcript_ENST00000563600.5, transcript_ENST00000564239.1, transcript_ENST00000565160.5, transcript_ENST00000566197.1, transcript_ENST00000566400.5, transcript_ENST00000566829.1, transcript_ENST00000566938.5, transcript_ENST00000567279.5, transcript_ENST00000568535.1, transcript_ENST00000569001.1, transcript_ENST00000569151.1, transcript_ENST00000569338.5, and transcript_ENST00000569440.5.

In some embodiments, the BBS4 pre-mRNA transcript is encoded by a genetic sequence with at least about 80%, 85%, 90%, 95%, 96%, 97%, 98%, 99% or 100% sequence identity to the Ensembl reference number ENSG00000140463.14 or a complement thereof. In some embodiments, the BBS4 pre-mRNA transcript comprises a sequence with at least about 80%, 85%, 90%, 95%, 96%, 97%, 98%, 99% or 100% sequence identity to a BBS4 pre-mRNA transcript or a complement thereof described herein.

In some embodiments, the targeted portion of the BBS4 pre-mRNA comprises a sequence with at least 80%, 85%, 90%, 95%, 97%, or 100% sequence identity to a region comprising at least 8 contiguous nucleic acids of a sequence of premrna_ENST00000268057.9, premrna_ENST00000395205.6, premrna_ENST00000561914.5, premrna_ENST00000562084.5, premrna_ENST00000562219.1, premrna_ENST00000563600.5, premrna_ENST00000564239.1, premrna_ENST00000565160.5, premrna_ENST00000566197.1, premrna_ENST00000566400.5, premrna_ENST00000566829.1, premrna_ENST00000566938.5, premrna_ENST00000567279.5, premrna_ENST00000568535.1, premrna_ENST00000569001.1, premrna_ENST00000569151.1, premrna_ENST00000569338.5, premrna_ENST00000569440.5 or a sequence of Table 3 or complements thereof. In some embodiments, the targeted portion of the BBS4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 302-SEQ ID NO: 309 or complements thereof. In some embodiments, the targeted portion of the BBS4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a BRD9 genomic sequence. In some embodiments, the ASO targets a NSAE pre-mRNA transcript from a BRD9 genomic sequence comprising a NSAE exon. In some embodiments, the ASO targets a NSAE pre-mRNA transcript from a BRD9 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000466684.5, premrna_ENST00000467963.6, premrna_ENST00000475706.5, premrna_ENST00000483173.5, premrna_ENST00000483234.5, premrna_ENST00000487688.1, premrna_ENST00000489093.1, premrna_ENST00000489816.5, premrna_ENST00000490814.6, premrna_ENST00000493082.5, premrna_ENST00000494422.1, premrna_ENST00000495265.5, premrna_ENST00000495794.5, premrna_ENST00000497410.5, premrna_ENST00000518250.5, premrna_ENST00000518251.1, premrna_ENST00000519112.5, premrna_ENST00000519838.5, and premrna_ENST00000523139.5. In some embodiments, the ASO targets a BRD9 pre-mRNA sequence comprising a NSAE exon. In some embodiments, the ASO targets a BRD9 pre-mRNA sequence comprising an intron flanking the 3′ splice site of the NSAE exon. In some embodiments, the ASO targets a BRD9 pre-mRNA sequence comprising an intron flanking the 5′ splice site of the NSAE exon. In some embodiments, the transcript is selected from the group consisting of transcript_ENST00000466684.5, transcript_ENST00000467963.6, transcript_ENST00000475706.5, transcript_ENST00000483173.5, transcript_ENST00000483234.5, transcript_ENST00000487688.1, transcript_ENST00000489093.1, transcript_ENST00000489816.5, transcript_ENST00000490814.6, transcript_ENST00000493082.5, transcript_ENST00000494422.1, transcript_ENST00000495265.5, transcript_ENST00000495794.5, transcript_ENST00000497410.5, transcript_ENST00000518250.5, transcript_ENST00000518251.1, transcript_ENST00000519112.5, transcript_ENST00000519838.5, and transcript_ENST00000523139.5.

In some embodiments, the BRD9 pre-mRNA transcript is encoded by a genetic sequence with at least about 80%, 85%, 90%, 95%, 96%, 97%, 98%, 99% or 100% sequence identity to the Ensembl reference number ENSG00000028310.18 or a complement thereof. In some embodiments, the BRD9 pre-mRNA transcript comprises a sequence with at least about 80%, 85%, 90%, 95%, 96%, 97%, 98%, 99% or 100% sequence identity to a BRD9 pre-mRNA transcript or a complement thereof described herein.

In some embodiments, the targeted portion of the BRD9 pre-mRNA comprises a sequence with at least 80%, 85%, 90%, 95%, 97%, or 100% sequence identity to a region comprising at least 8 contiguous nucleic acids of a sequence of premrna_ENST00000466684.5, premrna_ENST00000467963.6, premrna_ENST00000475706.5, premrna_ENST00000483173.5, premrna_ENST00000483234.5, premrna_ENST00000487688.1, premrna_ENST00000489093.1, premrna_ENST00000489816.5, premrna_ENST00000490814.6, premrna_ENST00000493082.5, premrna_ENST00000494422.1, premrna_ENST00000495265.5, premrna_ENST00000495794.5, premrna_ENST00000497410.5, premrna_ENST00000518250.5, premrna_ENST00000518251.1, premrna_ENST00000519112.5, premrna_ENST00000519838.5, premrna_ENST00000523139.5 or a sequence of Table 3 or complements thereof. In some embodiments, the targeted portion of the BRD9 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 310-SEQ ID NO: 313 or complements thereof. In some embodiments, the targeted portion of the BRD9 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CALM3 genomic sequence. In some embodiments, the ASO targets a NSAE pre-mRNA transcript from a CALM3 genomic sequence comprising a NSAE exon. In some embodiments, the ASO targets a NSAE pre-mRNA transcript from a CALM3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000291295.14, premrna_ENST00000391918.6, premrna_ENST00000477244.5, premrna_ENST00000482455.5, premrna_ENST00000486500.1, premrna_ENST00000594523.5, premrna_ENST00000595072.2, premrna_ENST00000596362.1, premrna_ENST00000597743.5, premrna_ENST00000597868.5, premrna_ENST00000598871.5, premrna_ENST00000599839.5, and premrna_ENST00000602169.2. In some embodiments, the ASO targets a CALM3 pre-mRNA sequence comprising a NSAE exon. In some embodiments, the ASO targets a CALM3 pre-mRNA sequence comprising an intron flanking the 3′ splice site of the NSAE exon. In some embodiments, the ASO targets a CALM3 pre-mRNA sequence comprising an intron flanking the 5′ splice site of the NSAE exon. In some embodiments, the transcript is selected from the group consisting of transcript_ENST00000291295.14, transcript_ENST00000391918.6, transcript_ENST00000477244.5, transcript_ENST00000482455.5, transcript_ENST00000486500.1, transcript_ENST00000594523.5, transcript_ENST00000595072.2, transcript_ENST00000596362.1, transcript_ENST00000597743.5, transcript_ENST00000597868.5, transcript_ENST00000598871.5, transcript_ENST00000599839.5, and transcript_ENST00000602169.2.

In some embodiments, the CALM3 pre-mRNA transcript is encoded by a genetic sequence with at least about 80%, 85%, 90%, 95%, 96%, 97%, 98%, 99% or 100% sequence identity to the Ensembl reference number ENSG00000160014.17 or a complement thereof. In some embodiments, the CALM3 pre-mRNA transcript comprises a sequence with at least about 80%, 85%, 90%, 95%, 96%, 97%, 98%, 99% or 100% sequence identity to a CALM3 pre-mRNA transcript or a complement thereof described herein.

In some embodiments, the targeted portion of the CALM3 pre-mRNA comprises a sequence with at least 80%, 85%, 90%, 95%, 97%, or 100% sequence identity to a region comprising at least 8 contiguous nucleic acids of a sequence of premrna_ENST00000291295.14, premrna_ENST00000391918.6, premrna_ENST00000477244.5, premrna_ENST00000482455.5, premrna_ENST00000486500.1, premrna_ENST00000594523.5, premrna_ENST00000595072.2, premrna_ENST00000596362.1, premrna_ENST00000597743.5, premrna_ENST00000597868.5, premrna_ENST00000598871.5, premrna_ENST00000599839.5, premrna_ENST00000602169.2 or a sequence of Table 3 or complements thereof. In some embodiments, the targeted portion of the CALM3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 314-SEQ ID NO: 317 or complements thereof. In some embodiments, the targeted portion of the CALM3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CARS1 genomic sequence. In some embodiments, the ASO targets a NSAE pre-mRNA transcript from a CARS1 genomic sequence comprising a NSAE exon. In some embodiments, the ASO targets a NSAE pre-mRNA transcript from a CARS1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000278224.13, premrna_ENST00000380525.8, premrna_ENST00000397111.9, premrna_ENST00000439280.6, premrna_ENST00000465207.5, premrna_ENST00000465240.1, premrna_ENST00000465331.1, premrna_ENST00000466442.6, premrna_ENST00000470221.6, premrna_ENST00000484484.5, premrna_ENST00000524825.5, premrna_ENST00000526890.5, premrna_ENST00000527330.2, premrna_ENST00000529772.5, premrna_ENST00000531387.5, and premrna_ENST00000639317.1. In some embodiments, the ASO targets a CARS1 pre-mRNA sequence comprising a NSAE exon. In some embodiments, the ASO targets a CARS1 pre-mRNA sequence comprising an intron flanking the 3′ splice site of the NSAE exon. In some embodiments, the ASO targets a CARS1 pre-mRNA sequence comprising an intron flanking the 5′ splice site of the NSAE exon. In some embodiments, the transcript is selected from the group consisting of transcript_ENST00000278224.13, transcript_ENST00000380525.8, transcript_ENST00000397111.9, transcript_ENST00000439280.6, transcript_ENST00000465207.5, transcript_ENST00000465240.1, transcript_ENST00000465331.1, transcript_ENST00000466442.6, transcript_ENST00000470221.6, transcript_ENST00000484484.5, transcript_ENST00000524825.5, transcript_ENST00000526890.5, transcript_ENST00000527330.2, transcript_ENST00000529772.5, transcript_ENST00000531387.5, and transcript_ENST00000639317.1.

In some embodiments, the CARS1 pre-mRNA transcript is encoded by a genetic sequence with at least about 80%, 85%, 90%, 95%, 96%, 97%, 98%, 99% or 100% sequence identity to the Ensembl reference number ENSG00000110619.17 or ENSG00000278191.4 or a complement thereof. In some embodiments, the CARS1 pre-mRNA transcript comprises a sequence with at least about 80%, 85%, 90%, 95%, 96%, 97%, 98%, 99% or 100% sequence identity to a CARS1 pre-mRNA transcript or a complement thereof described herein.

In some embodiments, the targeted portion of the CARS1 pre-mRNA comprises a sequence with at least 80%, 85%, 90%, 95%, 97%, or 100% sequence identity to a region comprising at least 8 contiguous nucleic acids of a sequence of premrna_ENST00000278224.13, premrna_ENST00000380525.8, premrna_ENST00000397111.9, premrna_ENST00000439280.6, premrna_ENST00000465207.5, premrna_ENST00000465240.1, premrna_ENST00000465331.1, premrna_ENST00000466442.6, premrna_ENST00000470221.6, premrna_ENST00000484484.5, premrna_ENST00000524825.5, premrna_ENST00000526890.5, premrna_ENST00000527330.2, premrna_ENST00000529772.5, premrna_ENST00000531387.5, premrna_ENST00000639317.1, or a sequence of Table 3 or complements thereof. In some embodiments, the targeted portion of the CARS1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 318-SEQ ID NO: 321 or complements thereof. In some embodiments, the targeted portion of the CARS1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CASP9 genomic sequence. In some embodiments, the ASO targets a NSAE pre-mRNA transcript from a CASP9 genomic sequence comprising a NSAE exon. In some embodiments, the ASO targets a NSAE pre-mRNA transcript from a CASP9 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000333868.10, premrna_ENST00000348549.9, premrna_ENST00000375890.8, premrna_ENST00000400777.7, premrna_ENST00000424908.5, premrna_ENST00000440484.1, premrna_ENST00000447522.5, premrna_ENST00000469637.1, premrna_ENST00000474305.2, premrna_ENST00000546424.5, and premrna_ENST00000546969.1. In some embodiments, the ASO targets a CASP9 pre-mRNA sequence comprising a NSAE exon. In some embodiments, the ASO targets a CASP9 pre-mRNA sequence comprising an intron flanking the 3′ splice site of the NSAE exon. In some embodiments, the ASO targets a CASP9 pre-mRNA sequence comprising an intron flanking the 5′ splice site of the NSAE exon. In some embodiments, the transcript is selected from the group consisting of transcript_ENST00000333868.10, transcript_ENST00000348549.9, transcript_ENST00000375890.8, transcript_ENST00000400777.7, transcript_ENST00000424908.5, transcript_ENST00000440484.1, transcript_ENST00000447522.5, transcript_ENST00000469637.1, transcript_ENST00000474305.2, transcript_ENST00000546424.5, and transcript_ENST00000546969.1.

In some embodiments, the CASP9 pre-mRNA transcript is encoded by a genetic sequence with at least about 80%, 85%, 90%, 95%, 96%, 97%, 98%, 99% or 100% sequence identity to the Ensembl reference number ENSG00000132906.18 or a complement thereof. In some embodiments, the CASP9 pre-mRNA transcript comprises a sequence with at least about 80%, 85%, 90%, 95%, 96%, 97%, 98%, 99% or 100% sequence identity to a CASP9 pre-mRNA transcript or a complement thereof described herein.

In some embodiments, the targeted portion of the CASP9 pre-mRNA comprises a sequence with at least 80%, 85%, 90%, 95%, 97%, or 100% sequence identity to a region comprising at least 8 contiguous nucleic acids of a sequence of premrna_ENST00000333868.10, premrna_ENST00000348549.9, premrna_ENST00000375890.8, premrna_ENST00000400777.7, premrna_ENST00000424908.5, premrna_ENST00000440484.1, premrna_ENST00000447522.5, premrna_ENST00000469637.1, premrna_ENST00000474305.2, premrna_ENST00000546424.5, premrna_ENST00000546969.1 or a sequence of Table 3 or complements thereof. In some embodiments, the targeted portion of the CASP9 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 322-SEQ ID NO: 325 or complements thereof. In some embodiments, the targeted portion of the CASP9 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CATSPER1 genomic sequence. In some embodiments, the ASO targets a NSAE pre-mRNA transcript from a CATSPER1 genomic sequence comprising a NSAE exon. In some embodiments, the ASO targets a NSAE pre-mRNA transcript from a CATSPER1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000312106.6 and premrna_ENST00000529244.1. In some embodiments, the ASO targets a CATSPER1 pre-mRNA sequence comprising a NSAE exon. In some embodiments, the ASO targets a CATSPER1 pre-mRNA sequence comprising an intron flanking the 3′ splice site of the NSAE exon. In some embodiments, the ASO targets a CATSPER1 pre-mRNA sequence comprising an intron flanking the 5′ splice site of the NSAE exon. In some embodiments, the transcript is selected from the group consisting of transcript_ENST00000312106.6 and transcript_ENST00000529244.1.

In some embodiments, the CATSPER1 pre-mRNA transcript is encoded by a genetic sequence with at least about 80%, 85%, 90%, 95%, 96%, 97%, 98%, 99% or 100% sequence identity to the Ensembl reference number ENSG00000175294.6 or a complement thereof. In some embodiments, the CATSPER1 pre-mRNA transcript comprises a sequence with at least about 80%, 85%, 90%, 95%, 96%, 97%, 98%, 99% or 100% sequence identity to a CATSPER1 pre-mRNA transcript or a complement thereof described herein.

In some embodiments, the targeted portion of the CATSPER1 pre-mRNA comprises a sequence with at least 80%, 85%, 90%, 95%, 97%, or 100% sequence identity to a region comprising at least 8 contiguous nucleic acids of a sequence of premrna_ENST00000312106.6, premrna_ENST00000529244.1, or a sequence of Table 3 or complements thereof. In some embodiments, the targeted portion of the CATSPER1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 326-SEQ ID NO: 329 or complements thereof. In some embodiments, the targeted portion of the CATSPER1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CCDC40 genomic sequence. In some embodiments, the ASO targets a NSAE pre-mRNA transcript from a CCDC40 genomic sequence comprising a NSAE exon. In some embodiments, the ASO targets a NSAE pre-mRNA transcript from a CCDC40 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000269318.9, premrna_ENST00000374876.4, premrna_ENST00000374877.7, premrna_ENST00000397545.9, premrna_ENST00000571028.1, premrna_ENST00000572083.5, premrna_ENST00000572253.5, premrna_ENST00000572270.1, premrna_ENST00000573474.5, premrna_ENST00000573903.1, premrna_ENST00000574099.1, premrna_ENST00000574799.5, premrna_ENST00000574933.1, premrna_ENST00000575431.1, premrna_ENST00000576033.5, and premrna_ENST00000576241.1. In some embodiments, the ASO targets a CCDC40 pre-mRNA sequence comprising a NSAE exon. In some embodiments, the ASO targets a CCDC40 pre-mRNA sequence comprising an intron flanking the 3′ splice site of the NSAE exon. In some embodiments, the ASO targets a CCDC40 pre-mRNA sequence comprising an intron flanking the 5′ splice site of the NSAE exon. In some embodiments, the transcript is selected from the group consisting of transcript_ENST00000269318.9, transcript_ENST00000374876.4, transcript_ENST00000374877.7, transcript_ENST00000397545.9, transcript_ENST00000571028.1, transcript_ENST00000572083.5, transcript_ENST00000572253.5, transcript_ENST00000572270.1, transcript_ENST00000573474.5, transcript_ENST00000573903.1, transcript_ENST00000574099.1, transcript_ENST00000574799.5, transcript_ENST00000574933.1, transcript_ENST00000575431.1, transcript_ENST00000576033.5, and transcript_ENST00000576241.1.

In some embodiments, the CCDC40 pre-mRNA transcript is encoded by a genetic sequence with at least about 80%, 85%, 90%, 95%, 96%, 97%, 98%, 99% or 100% sequence identity to the Ensembl reference number ENSG00000141519.15 or a complement thereof. In some embodiments, the CCDC40 pre-mRNA transcript comprises a sequence with at least about 80%, 85%, 90%, 95%, 96%, 97%, 98%, 99% or 100% sequence identity to a CCDC40 pre-mRNA transcript or a complement thereof described herein.

In some embodiments, the targeted portion of the CCDC40 pre-mRNA comprises a sequence with at least 80%, 85%, 90%, 95%, 97%, or 100% sequence identity to a region comprising at least 8 contiguous nucleic acids of a sequence of premrna_ENST00000269318.9, premrna_ENST00000374876.4, premrna_ENST00000374877.7, premrna_ENST00000397545.9, premrna_ENST00000571028.1, premrna_ENST00000572083.5, premrna_ENST00000572253.5, premrna_ENST00000572270.1, premrna_ENST00000573474.5, premrna_ENST00000573903.1, premrna_ENST00000574099.1, premrna_ENST00000574799.5, premrna_ENST00000574933.1, premrna_ENST00000575431.1, premrna_ENST00000576033.5, premrna_ENST00000576241.1, or a sequence of Table 3 or complements thereof. In some embodiments, the targeted portion of the CCDC40 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 330-SEQ ID NO: 333 or complements thereof. In some embodiments, the targeted portion of the CCDC40 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CCDC88B genomic sequence. In some embodiments, the ASO targets a NSAE pre-mRNA transcript from a CCDC88B genomic sequence comprising a NSAE exon. In some embodiments, the ASO targets a NSAE pre-mRNA transcript from a CCDC88B genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000301897.5, premrna_ENST00000356786.10, premrna_ENST00000359902.2, premrna_ENST00000463837.5, premrna_ENST00000472524.1, premrna_ENST00000473405.1, premrna_ENST00000479965.5, premrna_ENST00000492980.1, premma_ENST00000494080.5, and premrna_ENST00000494566.5. In some embodiments, the ASO targets a CCDC88B pre-mRNA sequence comprising a NSAE exon. In some embodiments, the ASO targets a CCDC88B pre-mRNA sequence comprising an intron flanking the 3′ splice site of the NSAE exon. In some embodiments, the ASO targets a CCDC88B pre-mRNA sequence comprising an intron flanking the 5′ splice site of the NSAE exon. In some embodiments, the transcript is selected from the group consisting of transcript_ENST00000301897.5, transcript_ENST00000356786.10, transcript_ENST00000359902.2, transcript_ENST00000463837.5, transcript_ENST00000472524.1, transcript_ENST00000473405.1, transcript_ENST00000479965.5, transcript_ENST00000492980.1, transcript_ENST00000494080.5, and transcript_ENST00000494566.5.

In some embodiments, the CCDC88B pre-mRNA transcript is encoded by a genetic sequence with at least about 80%, 85%, 90%, 95%, 96%, 97%, 98%, 99% or 100% sequence identity to the Ensembl reference number ENSG00000168071.22 or a complement thereof. In some embodiments, the CCDC88B pre-mRNA transcript comprises a sequence with at least about 80%, 85%, 90%, 95%, 96%, 97%, 98%, 99% or 100% sequence identity to a CCDC88B pre-mRNA transcript or a complement thereof described herein.

In some embodiments, the targeted portion of the CCDC88B pre-mRNA comprises a sequence with at least 80%, 85%, 90%, 95%, 97%, or 100% sequence identity to a region comprising at least 8 contiguous nucleic acids of a sequence of premrna_ENST00000301897.5, premrna_ENST00000356786.10, premrna_ENST00000359902.2, premrna_ENST00000463837.5, premma_ENST00000472524.1, premrna_ENST00000473405.1, premrna_ENST00000479965.5, premma_ENST00000492980.1, premrna_ENST00000494080.5, premrna_ENST00000494566.5 or a sequence of Table 3 or complements thereof. In some embodiments, the targeted portion of the CCDC88B pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 334-SEQ ID NO: 337 or complements thereof. In some embodiments, the targeted portion of the CCDC88B pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CD4 genomic sequence. In some embodiments, the ASO targets a NSAE pre-mRNA transcript from a CD4 genomic sequence comprising a NSAE exon. In some embodiments, the ASO targets a NSAE pre-mRNA transcript from a CD4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000011653.9, premrna_ENST00000437800.6, premma_ENST00000535466.5, premrna_ENST00000535707.5, premrna_ENST00000536563.1, premma_ENST00000536590.1, premrna_ENST00000536610.5, premrna_ENST00000538827.5, premma_ENST00000539492.1, premrna_ENST00000541982.5, premrna_ENST00000543755.1, and premma_ENST00000544344.5. In some embodiments, the ASO targets a CD4 pre-mRNA sequence comprising a NSAE exon. In some embodiments, the ASO targets a CD4 pre-mRNA sequence comprising an intron flanking the 3′ splice site of the NSAE exon. In some embodiments, the ASO targets a CD4 pre-mRNA sequence comprising an intron flanking the 5′ splice site of the NSAE exon. In some embodiments, the transcript is selected from the group consisting of transcript_ENST00000011653.9, transcript_ENST00000437800.6, transcript_ENST00000535466.5, transcript_ENST00000535707.5, transcript_ENST00000536563.1, transcript_ENST00000536590.1, transcript_ENST00000536610.5, transcript_ENST00000538827.5, transcript_ENST00000539492.1, transcript_ENST00000541982.5, transcript_ENST00000543755.1, and transcript_ENST00000544344.5.

In some embodiments, the CD4 pre-mRNA transcript is encoded by a genetic sequence with at least about 80%, 85%, 90%, 95%, 96%, 97%, 98%, 99% or 100% sequence identity to the Ensembl reference number ENSG00000010610.10 or a complement thereof. In some embodiments, the CD4 pre-mRNA transcript comprises a sequence with at least about 80%, 85%, 90%, 95%, 96%, 97%, 98%, 99% or 100% sequence identity to a CD4 pre-mRNA transcript or a complement thereof described herein.

In some embodiments, the targeted portion of the CD4 pre-mRNA comprises a sequence with at least 80%, 85%, 90%, 95%, 97%, or 100% sequence identity to a region comprising at least 8 contiguous nucleic acids of a sequence of premrna_ENST00000011653.9, premma_ENST00000437800.6, premrna_ENST00000535466.5, premrna_ENST00000535707.5, premma_ENST00000536563.1, premrna_ENST00000536590.1, premrna_ENST00000536610.5, premma_ENST00000538827.5, premrna_ENST00000539492.1, premrna_ENST00000541982.5, premma_ENST00000543755.1, premrna_ENST00000544344.5 or a sequence of Table 3 or complements thereof. In some embodiments, the targeted portion of the CD4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 338-SEQ ID NO: 341 or complements thereof. In some embodiments, the targeted portion of the CD4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CD58 genomic sequence. In some embodiments, the ASO targets a NSAE pre-mRNA transcript from a CD58 genomic sequence comprising a NSAE exon. In some embodiments, the ASO targets a NSAE pre-mRNA transcript from a CD58 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000369487.3, premrna_ENST00000369489.10, premma_ENST00000457047.6, premrna_ENST00000464088.5, and premma_ENST00000526981.1. In some embodiments, the ASO targets a CD58 pre-mRNA sequence comprising a NSAE exon. In some embodiments, the ASO targets a CD58 pre-mRNA sequence comprising an intron flanking the 3′ splice site of the NSAE exon. In some embodiments, the ASO targets a CD58 pre-mRNA sequence comprising an intron flanking the 5′ splice site of the NSAE exon. In some embodiments, the transcript is selected from the group consisting of transcript_ENST00000369487.3, transcript_ENST00000369489.10, transcript_ENST00000457047.6, transcript_ENST00000464088.5, and transcript_ENST00000526981.1.

In some embodiments, the CD58 pre-mRNA transcript is encoded by a genetic sequence with at least about 80%, 85%, 90%, 95%, 96%, 97%, 98%, 99% or 100% sequence identity to the Ensembl reference number ENSG00000116815.16 or a complement thereof. In some embodiments, the CD58 pre-mRNA transcript comprises a sequence with at least about 80%, 85%, 90%, 95%, 96%, 97%, 98%, 99% or 100% sequence identity to a CD58 pre-mRNA transcript or a complement thereof described herein.

In some embodiments, the targeted portion of the CD58 pre-mRNA comprises a sequence with at least 80%, 85%, 90%, 95%, 97%, or 100% sequence identity to a region comprising at least 8 contiguous nucleic acids of a sequence of premrna_ENST00000369487.3, premrna_ENST00000369489.10, premrna_ENST00000457047.6, premrna_ENST00000464088.5, premrna_ENST00000526981.1 or a sequence of Table 3 or complements thereof. In some embodiments, the targeted portion of the CD58 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 342-SEQ ID NO: 347 or complements thereof. In some embodiments, the targeted portion of the CD58 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CES2 genomic sequence. In some embodiments, the ASO targets a NSAE pre-mRNA transcript from a CES2 genomic sequence comprising a NSAE exon. In some embodiments, the ASO targets a NSAE pre-mRNA transcript from a CES2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000317091.10, premrna_ENST00000417689.6, premrna_ENST00000561697.5, premrna_ENST00000561843.1, premrna_ENST00000563988.1, premrna_ENST00000564420.1, premrna_ENST00000566182.1, premrna_ENST00000566359.1, premrna_ENST00000567128.1, premrna_ENST00000568347.1, premrna_ENST00000568470.6, premrna_ENST00000570032.2, premrna_ENST00000652196.1, and premrna_ENST00000652667.1.

In some embodiments, the ASO targets a CES2 pre-mRNA sequence comprising a NSAE exon. In some embodiments, the ASO targets a CES2 pre-mRNA sequence comprising an intron flanking the 3′ splice site of the NSAE exon. In some embodiments, the ASO targets a CES2 pre-mRNA sequence comprising an intron flanking the 5′ splice site of the NSAE exon. In some embodiments, the transcript is selected from the group consisting of transcript_ENST00000317091.10, transcript_ENST00000417689.6, transcript_ENST00000561697.5, transcript_ENST00000561843.1, transcript_ENST00000563988.1, transcript_ENST00000564420.1, transcript_ENST00000566182.1, transcript_ENST00000566359.1, transcript_ENST00000567128.1, transcript_ENST00000568347.1, transcript_ENST00000568470.6, transcript_ENST00000570032.2, transcript_ENST00000652196.1, and transcript_ENST00000652667.1.

In some embodiments, the CES2 pre-mRNA transcript is encoded by a genetic sequence with at least about 80%, 85%, 90%, 95%, 96%, 97%, 98%, 99% or 100% sequence identity to the Ensembl reference number ENSG00000172831.14 or a complement thereof. In some embodiments, the CES2 pre-mRNA transcript comprises a sequence with at least about 80%, 85%, 90%, 95%, 96%, 97%, 98%, 99% or 100% sequence identity to a CES2 pre-mRNA transcript or a complement thereof described herein.

In some embodiments, the targeted portion of the CES2 pre-mRNA comprises a sequence with at least 80%, 85%, 90%, 95%, 97%, or 100% sequence identity to a region comprising at least 8 contiguous nucleic acids of a sequence of premrna_ENST00000317091.10, premrna_ENST00000417689.6, premrna_ENST00000561697.5, premrna_ENST00000561843.1, premrna_ENST00000563988.1, premrna_ENST00000564420.1, premrna_ENST00000566182.1, premrna_ENST00000566359.1, premrna_ENST00000567128.1, premrna_ENST00000568347.1, premrna_ENST00000568470.6, premrna_ENST00000570032.2, premrna_ENST00000652196.1, premrna_ENST00000652667.1, or a sequence of Table 3 or complements thereof. In some embodiments, the targeted portion of the CES2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 348-SEQ ID NO: 359 or complements thereof. In some embodiments, the targeted portion of the CES2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CHFR genomic sequence. In some embodiments, the ASO targets a NSAE pre-mRNA transcript from a CHFR genomic sequence comprising a NSAE exon. In some embodiments, the ASO targets a NSAE pre-mRNA transcript from a CHFR genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000266880.11, premrna_ENST00000315585.11, premrna_ENST00000432561.6, premrna_ENST00000443047.6, premrna_ENST00000450056.6, premrna_ENST00000499045.2, premrna_ENST00000502279.2, premrna_ENST00000511001.2, premrna_ENST00000535181.5, premrna_ENST00000535527.5, premrna_ENST00000535897.1, premrna_ENST00000536196.1, premrna_ENST00000536843.5, premrna_ENST00000536932.5, premrna_ENST00000537551.5, premrna_ENST00000538235.2, premrna_ENST00000540537.5, premrna_ENST00000540963.1, premrna_ENST00000541341.5, premrna_ENST00000541817.5, premrna_ENST00000542714.5, premrna_ENST00000544093.5, premrna_ENST00000544268.5, and premrna_ENST00000545046.5. In some embodiments, the ASO targets a CHFR pre-mRNA sequence comprising a NSAE exon. In some embodiments, the ASO targets a CHFR pre-mRNA sequence comprising an intron flanking the 3′ splice site of the NSAE exon. In some embodiments, the ASO targets a CHFR pre-mRNA sequence comprising an intron flanking the 5′ splice site of the NSAE exon. In some embodiments, the transcript is selected from the group consisting of transcript_ENST00000266880.11, transcript_ENST00000315585.11, transcript_ENST00000432561.6, transcript_ENST00000443047.6, transcript_ENST00000450056.6, transcript_ENST00000499045.2, transcript_ENST00000502279.2, transcript_ENST00000511001.2, transcript_ENST00000535181.5, transcript_ENST00000535527.5, transcript_ENST00000535897.1, transcript_ENST00000536196.1, transcript_ENST00000536843.5, transcript_ENST00000536932.5, transcript_ENST00000537551.5, transcript_ENST00000538235.2, transcript_ENST00000540537.5, transcript_ENST00000540963.1, transcript_ENST00000541341.5, transcript_ENST00000541817.5, transcript_ENST00000542714.5, transcript_ENST00000544093.5, transcript_ENST00000544268.5, and transcript_ENST00000545046.5.

In some embodiments, the CHFR pre-mRNA transcript is encoded by a genetic sequence with at least about 80%, 85%, 90%, 95%, 96%, 97%, 98%, 99% or 100% sequence identity to the Ensembl reference number ENSG00000072609.17 or a complement thereof. In some embodiments, the CHFR pre-mRNA transcript comprises a sequence with at least about 80%, 85%, 90%, 95%, 96%, 97%, 98%, 99% or 100% sequence identity to a CHFR pre-mRNA transcript or a complement thereof described herein.

In some embodiments, the targeted portion of the CHFR pre-mRNA comprises a sequence with at least 80%, 85%, 90%, 95%, 97%, or 100% sequence identity to a region comprising at least 8 contiguous nucleic acids of a sequence of premrna_ENST00000266880.11, premrna_ENST00000315585.11, premrna_ENST00000432561.6, premrna_ENST00000443047.6, premrna_ENST00000450056.6, premrna_ENST00000499045.2, premrna_ENST00000502279.2, premrna_ENST00000511001.2, premrna_ENST00000535181.5, premrna_ENST00000535527.5, premrna_ENST00000535897.1, premrna_ENST00000536196.1, premrna_ENST00000536843.5, premrna_ENST00000536932.5, premrna_ENST00000537551.5, premrna_ENST00000538235.2, premrna_ENST00000540537.5, premrna_ENST00000540963.1, premrna_ENST00000541341.5, premrna_ENST00000541817.5, premrna_ENST00000542714.5, premrna_ENST00000544093.5, premrna_ENST00000544268.5, premrna_ENST00000545046.5 or a sequence of Table 3 or complements thereof. In some embodiments, the targeted portion of the CHFR pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 360-SEQ ID NO: 363 or complements thereof. In some embodiments, the targeted portion of the CHFR pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CLCN2 genomic sequence. In some embodiments, the ASO targets a NSAE pre-mRNA transcript from a CLCN2 genomic sequence comprising a NSAE exon. In some embodiments, the ASO targets a NSAE pre-mRNA transcript from a CLCN2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000265593.9, premrna_ENST00000344937.11, premrna_ENST00000430397.5, premrna_ENST00000434054.6, premrna_ENST00000457512.1, premrna_ENST00000465231.1, premrna_ENST00000475279.2, premrna_ENST00000485667.1, premrna_ENST00000491162.1, premrna_ENST00000636180.1, premrna_ENST00000636241.1, premrna_ENST00000636419.1, premrna_ENST00000636492.1, premrna_ENST00000636658.1, premrna_ENST00000636661.1, premrna_ENST00000636830.1, premrna_ENST00000636860.1, premrna_ENST00000637258.1, premrna_ENST00000637392.1, premrna_ENST00000637538.1, premrna_ENST00000637909.1, and premrna_ENST00000638134.1. In some embodiments, the ASO targets a CLCN2 pre-mRNA sequence comprising a NSAE exon. In some embodiments, the ASO targets a CLCN2 pre-mRNA sequence comprising an intron flanking the 3′ splice site of the NSAE exon. In some embodiments, the ASO targets a CLCN2 pre-mRNA sequence comprising an intron flanking the 5′ splice site of the NSAE exon. In some embodiments, the transcript is selected from the group consisting of transcript_ENST00000265593.9, transcript_ENST00000344937.11, transcript_ENST00000430397.5, transcript_ENST00000434054.6, transcript_ENST00000457512.1, transcript_ENST00000465231.1, transcript_ENST00000475279.2, transcript_ENST00000485667.1, transcript_ENST00000491162.1, transcript_ENST00000636180.1, transcript_ENST00000636241.1, transcript_ENST00000636419.1, transcript_ENST00000636492.1, transcript_ENST00000636658.1, transcript_ENST00000636661.1, transcript_ENST00000636830.1, transcript_ENST00000636860.1, transcript_ENST00000637258.1, transcript_ENST00000637392.1, transcript_ENST00000637538.1, transcript_ENST00000637909.1, and transcript_ENST00000638134.1.

In some embodiments, the CLCN2 pre-mRNA transcript is encoded by a genetic sequence with at least about 80%, 85%, 90%, 95%, 96%, 97%, 98%, 99% or 100% sequence identity to the Ensembl reference number ENSG00000114859.16 or a complement thereof. In some embodiments, the CLCN2 pre-mRNA transcript comprises a sequence with at least about 80%, 85%, 90%, 95%, 96%, 97%, 98%, 99% or 100% sequence identity to a CLCN2 pre-mRNA transcript or a complement thereof described herein.

In some embodiments, the targeted portion of the CLCN2 pre-mRNA comprises a sequence with at least 80%, 85%, 90%, 95%, 97%, or 100% sequence identity to a region comprising at least 8 contiguous nucleic acids of a sequence of premrna_ENST00000265593.9, premrna_ENST00000344937.11, premrna_ENST00000430397.5, premrna_ENST00000434054.6, premrna_ENST00000457512.1, premrna_ENST00000465231.1, premrna_ENST00000475279.2, premrna_ENST00000485667.1, premrna_ENST00000491162.1, premrna_ENST00000636180.1, premrna_ENST00000636241.1, premrna_ENST00000636419.1, premrna_ENST00000636492.1, premrna_ENST00000636658.1, premrna_ENST00000636661.1, premrna_ENST00000636830.1, premrna_ENST00000636860.1, premrna_ENST00000637258.1, premrna_ENST00000637392.1, premrna_ENST00000637538.1, premrna_ENST00000637909.1, premrna_ENST00000638134.1 or a sequence of Table 3 or complements thereof. In some embodiments, the targeted portion of the CLCN2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 364-SEQ ID NO: 369 or complements thereof. In some embodiments, the targeted portion of the CLCN2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CNOT3 genomic sequence. In some embodiments, the ASO targets a NSAE pre-mRNA transcript from a CNOT3 genomic sequence comprising a NSAE exon. In some embodiments, the ASO targets a NSAE pre-mRNA transcript from a CNOT3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000221232.11, premrna_ENST00000358389.7, premrna_ENST00000440571.6, premrna_ENST00000447684.5, premrna_ENST00000457463.1, premrna_ENST00000471126.1, premrna_ENST00000496327.2, premrna_ENST00000613073.4, premrna_ENST00000617930.2, premrna_ENST00000618939.5, premrna_ENST00000642159.1, premrna_ENST00000644245.1, premrna_ENST00000644707.1, premrna_ENST00000646002.1, and premrna_ENST00000647082.1. In some embodiments, the ASO targets a CNOT3 pre-mRNA sequence comprising a NSAE exon. In some embodiments, the ASO targets a CNOT3 pre-mRNA sequence comprising an intron flanking the 3′ splice site of the NSAE exon. In some embodiments, the ASO targets a CNOT3 pre-mRNA sequence comprising an intron flanking the 5′ splice site of the NSAE exon. In some embodiments, the transcript is selected from the group consisting of transcript_ENST00000221232.11, transcript_ENST00000358389.7, transcript_ENST00000440571.6, transcript_ENST00000447684.5, transcript_ENST00000457463.1, transcript_ENST00000471126.1, transcript_ENST00000496327.2, transcript_ENST00000613073.4, transcript_ENST00000617930.2, transcript_ENST00000618939.5, transcript_ENST00000642159.1, transcript_ENST00000644245.1, transcript_ENST00000644707.1, transcript_ENST00000646002.1, and transcript_ENST00000647082.1.

In some embodiments, the CNOT3 pre-mRNA transcript is encoded by a genetic sequence with at least about 80%, 85%, 90%, 95%, 96%, 97%, 98%, 99% or 100% sequence identity to the Ensembl reference number ENSG00000088038.19, ENSG00000273943.4, ENSG00000274176.4, ENSG00000274616.4, ENSG00000274941.4, ENSG00000275979.4, ENSG00000276082.4, ENSG00000277600.4, ENSG00000277114.4, or ENSG00000277615.4, or a complement thereof. In some embodiments, the CNOT3 pre-mRNA transcript comprises a sequence with at least about 80%, 85%, 90%, 95%, 96%, 97%, 98%, 99% or 100% sequence identity to a CNOT3 pre-mRNA transcript or a complement thereof described herein.

In some embodiments, the targeted portion of the CNOT3 pre-mRNA comprises a sequence with at least 80%, 85%, 90%, 95%, 97%, or 100% sequence identity to a region comprising at least 8 contiguous nucleic acids of a sequence of premrna_ENST00000221232.11, premrna_ENST00000358389.7, premrna_ENST00000440571.6, premrna_ENST00000447684.5, premrna_ENST00000457463.1, premrna_ENST00000471126.1, premrna_ENST00000496327.2, premrna_ENST00000613073.4, premrna_ENST00000617930.2, premrna_ENST00000618939.5, premrna_ENST00000642159.1, premrna_ENST00000644245.1, premrna_ENST00000644707.1, premrna_ENST00000646002.1, premrna_ENST00000647082.1 or a sequence of Table 3 or complements thereof. In some embodiments, the targeted portion of the CNOT3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 370-SEQ ID NO: 377 or complements thereof. In some embodiments, the targeted portion of the CNOT3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CNTROB genomic sequence. In some embodiments, the ASO targets a NSAE pre-mRNA transcript from a CNTROB genomic sequence comprising a NSAE exon. In some embodiments, the ASO targets a NSAE pre-mRNA transcript from a CNTROB genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000380262.7, premrna_ENST00000563694.6, premrna_ENST00000565740.5, premrna_ENST00000570782.1, premrna_ENST00000570784.1, premrna_ENST00000571540.5, premrna_ENST00000571632.5, premrna_ENST00000573862.1, premrna_ENST00000574430.5, premrna_ENST00000575408.5, premrna_ENST00000576536.1, premrna_ENST00000576538.5, premrna_ENST00000576587.1, premrna_ENST00000576723.5, and premrna_ENST00000576922.5. In some embodiments, the ASO targets a CNTROB pre-mRNA sequence comprising a NSAE exon. In some embodiments, the ASO targets a CNTROB pre-mRNA sequence comprising an intron flanking the 3′ splice site of the NSAE exon. In some embodiments, the ASO targets a CNTROB pre-mRNA sequence comprising an intron flanking the 5′ splice site of the NSAE exon. In some embodiments, the transcript is selected from the group consisting of transcript_ENST00000380262.7, transcript_ENST00000563694.6, transcript_ENST00000565740.5, transcript_ENST00000570782.1, transcript_ENST00000570784.1, transcript_ENST00000571540.5, transcript_ENST00000571632.5, transcript_ENST00000573862.1, transcript_ENST00000574430.5, transcript_ENST00000575408.5, transcript_ENST00000576536.1, transcript_ENST00000576538.5, transcript_ENST00000576587.1, transcript_ENST00000576723.5, and transcript_ENST00000576922.5.

In some embodiments, the CNTROB pre-mRNA transcript is encoded by a genetic sequence with at least about 80%, 85%, 90%, 95%, 96%, 97%, 98%, 99% or 100% sequence identity to the Ensembl reference number ENSG00000170037.14 or a complement thereof. In some embodiments, the CNTROB pre-mRNA transcript comprises a sequence with at least about 80%, 85%, 90%, 95%, 96%, 97%, 98%, 99% or 100% sequence identity to a CNTROB pre-mRNA transcript or a complement thereof described herein.

In some embodiments, the targeted portion of the CNTROB pre-mRNA comprises a sequence with at least 80%, 85%, 90%, 95%, 97%, or 100% sequence identity to a region comprising at least 8 contiguous nucleic acids of a sequence of premrna_ENST00000380262.7, premrna_ENST00000563694.6, premrna_ENST00000565740.5, premrna_ENST00000570782.1, premrna_ENST00000570784.1, premrna_ENST00000571540.5, premrna_ENST00000571632.5, premrna_ENST00000573862.1, premrna_ENST00000574430.5, premrna_ENST00000575408.5, premrna_ENST00000576536.1, premrna_ENST00000576538.5, premrna_ENST00000576587.1, premrna_ENST00000576723.5, premrna_ENST00000576922.5 or a sequence of Table 3 or complements thereof. In some embodiments, the targeted portion of the CNTROB pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 378-SEQ ID NO: 381 or complements thereof. In some embodiments, the targeted portion of the CNTROB pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a COL5A3 genomic sequence. In some embodiments, the ASO targets a NSAE pre-mRNA transcript from a COL5A3 genomic sequence comprising a NSAE exon. In some embodiments, the ASO targets a NSAE pre-mRNA transcript from a COL5A3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000264828.4 and premrna_ENST00000461214.1. In some embodiments, the ASO targets a COL5A3 pre-mRNA sequence comprising a NSAE exon. In some embodiments, the ASO targets a COL5A3 pre-mRNA sequence comprising an intron flanking the 3′ splice site of the NSAE exon. In some embodiments, the ASO targets a COL5A3 pre-mRNA sequence comprising an intron flanking the 5′ splice site of the NSAE exon. In some embodiments, the transcript is selected from the group consisting of transcript_ENST00000264828.4 and transcript_ENST00000461214.1.

In some embodiments, the COL5A3 pre-mRNA transcript is encoded by a genetic sequence with at least about 80%, 85%, 90%, 95%, 96%, 97%, 98%, 99% or 100% sequence identity to the Ensembl reference number ENSG00000080573.7 or a complement thereof. In some embodiments, the COL5A3 pre-mRNA transcript comprises a sequence with at least about 80%, 85%, 90%, 95%, 96%, 97%, 98%, 99% or 100% sequence identity to a COL5A3 pre-mRNA transcript or a complement thereof described herein.

In some embodiments, the targeted portion of the COL5A3 pre-mRNA comprises a sequence with at least 80%, 85%, 90%, 95%, 97%, or 100% sequence identity to a region comprising at least 8 contiguous nucleic acids of a sequence of premrna_ENST00000264828.4, premrna_ENST00000461214.1, or a sequence of Table 3 or complements thereof. In some embodiments, the targeted portion of the COL5A3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 382-SEQ ID NO: 385 or complements thereof. In some embodiments, the targeted portion of the COL5A3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CPSF1 genomic sequence. In some embodiments, the ASO targets a NSAE pre-mRNA transcript from a CPSF1 genomic sequence comprising a NSAE exon. In some embodiments, the ASO targets a NSAE pre-mRNA transcript from a CPSF1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000526271.2, premrna_ENST00000527827.1, premrna_ENST00000527916.1, premrna_ENST00000529288.1, premrna_ENST00000531042.5, premrna_ENST00000531480.2, premrna_ENST00000531727.5, premrna_ENST00000532560.5, premrna_ENST00000532725.1, premrna_ENST00000532935.5, premrna_ENST00000533492.1, premrna_ENST00000616140.2, premrna_ENST00000620219.4, and premrna_ENST00000622776.1. In some embodiments, the ASO targets a CPSF1 pre-mRNA sequence comprising a NSAE exon. In some embodiments, the ASO targets a CPSF1 pre-mRNA sequence comprising an intron flanking the 3′ splice site of the NSAE exon. In some embodiments, the ASO targets a CPSF1 pre-mRNA sequence comprising an intron flanking the 5′ splice site of the NSAE exon. In some embodiments, the transcript is selected from the group consisting of transcript_ENST00000526271.2, transcript_ENST00000527827.1, transcript_ENST00000527916.1, transcript_ENST00000529288.1, transcript_ENST00000531042.5, transcript_ENST00000531480.2, transcript_ENST00000531727.5, transcript_ENST00000532560.5, transcript_ENST00000532725.1, transcript_ENST00000532935.5, transcript_ENST00000533492.1, transcript_ENST00000616140.2, transcript_ENST00000620219.4, and transcript_ENST00000622776.1.

In some embodiments, the CPSF1 pre-mRNA transcript is encoded by a genetic sequence with at least about 80%, 85%, 90%, 95%, 96%, 97%, 98%, 99% or 100% sequence identity to the Ensembl reference number ENSG00000071894.17 or ENSG00000285049.2 or a complement thereof. In some embodiments, the CPSF1 pre-mRNA transcript comprises a sequence with at least about 80%, 85%, 90%, 95%, 96%, 97%, 98%, 99% or 100% sequence identity to a CPSF1 pre-mRNA transcript or a complement thereof described herein.

In some embodiments, the targeted portion of the CPSF1 pre-mRNA comprises a sequence with at least 80%, 85%, 90%, 95%, 97%, or 100% sequence identity to a region comprising at least 8 contiguous nucleic acids of a sequence of premrna_ENST00000526271.2, premrna_ENST00000527827.1, premrna_ENST00000527916.1, premrna_ENST00000529288.1, premrna_ENST00000531042.5, premrna_ENST00000531480.2, premrna_ENST00000531727.5, premrna_ENST00000532560.5, premrna_ENST00000532725.1, premrna_ENST00000532935.5, premrna_ENST00000533492.1, premrna_ENST00000616140.2, premrna_ENST00000620219.4, premrna_ENST00000622776.1, or a sequence of Table 3 or complements thereof. In some embodiments, the targeted portion of the CPSF1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 386-SEQ ID NO: 389 or complements thereof. In some embodiments, the targeted portion of the CPSF1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CSPP1 genomic sequence. In some embodiments, the ASO targets a NSAE pre-mRNA transcript from a CSPP1 genomic sequence comprising a NSAE exon. In some embodiments, the ASO targets a NSAE pre-mRNA transcript from a CSPP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000262210.9, premrna_ENST00000519163.6, premrna_ENST00000519668.1, premrna_ENST00000519701.5, premrna_ENST00000521168.5, premrna_ENST00000521324.1, and premrna_ENST00000521919.5. In some embodiments, the ASO targets a CSPP1 pre-mRNA sequence comprising a NSAE exon. In some embodiments, the ASO targets a CSPP1 pre-mRNA sequence comprising an intron flanking the 3′ splice site of the NSAE exon. In some embodiments, the ASO targets a CSPP1 pre-mRNA sequence comprising an intron flanking the 5′ splice site of the NSAE exon. In some embodiments, the transcript is selected from the group consisting of transcript_ENST00000262210.9, transcript_ENST00000519163.6, transcript_ENST00000519668.1, transcript_ENST00000519701.5, transcript_ENST00000521168.5, transcript_ENST00000521324.1, and transcript_ENST00000521919.5.

In some embodiments, the CSPP1 pre-mRNA transcript is encoded by a genetic sequence with at least about 80%, 85%, 90%, 95%, 96%, 97%, 98%, 99% or 100% sequence identity to the Ensembl reference number ENSG00000104218.14 or a complement thereof. In some embodiments, the CSPP1 pre-mRNA transcript comprises a sequence with at least about 80%, 85%, 90%, 95%, 96%, 97%, 98%, 99% or 100% sequence identity to a CSPP1 pre-mRNA transcript or a complement thereof described herein.

In some embodiments, the targeted portion of the CSPP1 pre-mRNA comprises a sequence with at least 80%, 85%, 90%, 95%, 97%, or 100% sequence identity to a region comprising at least 8 contiguous nucleic acids of a sequence of premrna_ENST00000262210.9, premrna_ENST00000519163.6, premrna_ENST00000519668.1, premrna_ENST00000519701.5, premrna_ENST00000521168.5, premrna_ENST00000521324.1, and premrna_ENST00000521919.5, or a sequence of Table 3 or complements thereof. In some embodiments, the targeted portion of the CSPP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 390-SEQ ID NO: 393 or complements thereof. In some embodiments, the targeted portion of the CSPP1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CTF1 genomic sequence. In some embodiments, the ASO targets a NSAE pre-mRNA transcript from a CTF1 genomic sequence comprising a NSAE exon. In some embodiments, the ASO targets a NSAE pre-mRNA transcript from a CTF1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000279804.2, and premrna_ENST00000395019.3. In some embodiments, the ASO targets a CTF1 pre-mRNA sequence comprising a NSAE exon. In some embodiments, the ASO targets a CTF1 pre-mRNA sequence comprising an intron flanking the 3′ splice site of the NSAE exon. In some embodiments, the ASO targets a CTF1 pre-mRNA sequence comprising an intron flanking the 5′ splice site of the NSAE exon. In some embodiments, the transcript is selected from the group consisting of transcript_ENST00000279804.2, and transcript_ENST00000395019.3.

In some embodiments, the CTF1 pre-mRNA transcript is encoded by a genetic sequence with at least about 80%, 85%, 90%, 95%, 96%, 97%, 98%, 99% or 100% sequence identity to the Ensembl reference number ENSG00000150281.6 or a complement thereof. In some embodiments, the CTF1 pre-mRNA transcript comprises a sequence with at least about 80%, 85%, 90%, 95%, 96%, 97%, 98%, 99% or 100% sequence identity to a CTF1 pre-mRNA transcript or a complement thereof described herein.

In some embodiments, the targeted portion of the CTF1 pre-mRNA comprises a sequence with at least 80%, 85%, 90%, 95%, 97%, or 100% sequence identity to a region comprising at least 8 contiguous nucleic acids of a sequence of premrna_ENST00000279804.2, premrna_ENST00000395019.3 or a sequence of Table 3 or complements thereof. In some embodiments, the targeted portion of the CTF1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 394-SEQ ID NO: 399 or complements thereof. In some embodiments, the targeted portion of the CTF1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CTH genomic sequence. In some embodiments, the ASO targets a NSAE pre-mRNA transcript from a CTH genomic sequence comprising a NSAE exon. In some embodiments, the ASO targets a NSAE pre-mRNA transcript from a CTH genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000346806.2, premrna_ENST00000370938.8, premrna_ENST00000411986.6, premrna_ENST00000464926.1, and premrna_ENST00000482383.1. In some embodiments, the ASO targets a CTH pre-mRNA sequence comprising a NSAE exon. In some embodiments, the ASO targets a CTH pre-mRNA sequence comprising an intron flanking the 3′ splice site of the NSAE exon. In some embodiments, the ASO targets a CTH pre-mRNA sequence comprising an intron flanking the 5′ splice site of the NSAE exon. In some embodiments, the transcript is selected from the group consisting of transcript_ENST00000346806.2, transcript_ENST00000370938.8, transcript_ENST00000411986.6, transcript_ENST00000464926.1, and transcript_ENST00000482383.1.

In some embodiments, the CTH pre-mRNA transcript is encoded by a genetic sequence with at least about 80%, 85%, 90%, 95%, 96%, 97%, 98%, 99% or 100% sequence identity to the Ensembl reference number ENSG00000116761.12 or a complement thereof. In some embodiments, the CTH pre-mRNA transcript comprises a sequence with at least about 80%, 85%, 90%, 95%, 96%, 97%, 98%, 99% or 100% sequence identity to a CTH pre-mRNA transcript or a complement thereof described herein.

In some embodiments, the targeted portion of the CTH pre-mRNA comprises a sequence with at least 80%, 85%, 90%, 95%, 97%, or 100% sequence identity to a region comprising at least 8 contiguous nucleic acids of a sequence of premrna_ENST00000346806.2, premrna_ENST00000370938.8, premrna_ENST00000411986.6, premrna_ENST00000464926.1, premrna_ENST00000482383.1 or a sequence of Table 3 or complements thereof. In some embodiments, the targeted portion of the CTH pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 400-SEQ ID NO: 407 or complements thereof. In some embodiments, the targeted portion of the CTH pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a CYP3A5 genomic sequence. In some embodiments, the ASO targets a NSAE pre-mRNA transcript from a CYP3A5 genomic sequence comprising a NSAE exon. In some embodiments, the ASO targets a NSAE pre-mRNA transcript from a CYP3A5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000222982.8, premrna_ENST00000339843.6, premrna_ENST00000439761.3, premrna_ENST00000456417.5, premrna_ENST00000461920.5, premrna_ENST00000463364.5, premrna_ENST00000463907.5, premrna_ENST00000466061.5, premrna_ENST00000469622.5, premrna_ENST00000469887.5, premrna_ENST00000473347.1, premrna_ENST00000480723.5, premrna_ENST00000481825.5, premrna_ENST00000488187.1, premrna_ENST00000489231.1, and premrna_ENST00000646887.1. In some embodiments, the ASO targets a CYP3A5 pre-mRNA sequence comprising a NSAE exon. In some embodiments, the ASO targets a CYP3A5 pre-mRNA sequence comprising an intron flanking the 3′ splice site of the NSAE exon. In some embodiments, the ASO targets a CYP3A5 pre-mRNA sequence comprising an intron flanking the 5′ splice site of the NSAE exon. In some embodiments, the transcript is selected from the group consisting of transcript_ENST00000222982.8, transcript_ENST00000339843.6, transcript_ENST00000439761.3, transcript_ENST00000456417.5, transcript_ENST00000461920.5, transcript_ENST00000463364.5, transcript_ENST00000463907.5, transcript_ENST00000466061.5, transcript_ENST00000469622.5, transcript_ENST00000469887.5, transcript_ENST00000473347.1, transcript_ENST00000480723.5, transcript_ENST00000481825.5, transcript_ENST00000488187.1, transcript_ENST00000489231.1, and transcript_ENST00000646887.1.

In some embodiments, the CYP3A5 pre-mRNA transcript is encoded by a genetic sequence with at least about 80%, 85%, 90%, 95%, 96%, 97%, 98%, 99% or 100% sequence identity to the Ensembl reference number ENSG00000106258.15 or a complement thereof. In some embodiments, the CYP3A5 pre-mRNA transcript comprises a sequence with at least about 80%, 85%, 90%, 95%, 96%, 97%, 98%, 99% or 100% sequence identity to a CYP3A5 pre-mRNA transcript or a complement thereof described herein.

In some embodiments, the targeted portion of the CYP3A5 pre-mRNA comprises a sequence with at least 80%, 85%, 90%, 95%, 97%, or 100% sequence identity to a region comprising at least 8 contiguous nucleic acids of a sequence of premrna_ENST00000222982.8, premrna_ENST00000339843.6, premrna_ENST00000439761.3, premrna_ENST00000456417.5, premrna_ENST00000461920.5, premrna_ENST00000463364.5, premrna_ENST00000463907.5, premrna_ENST00000466061.5, premrna_ENST00000469622.5, premrna_ENST00000469887.5, premrna_ENST00000473347.1, premrna_ENST00000480723.5, premrna_ENST00000481825.5, premrna_ENST00000488187.1, premrna_ENST00000489231.1, premrna_ENST00000646887.1 or a sequence of Table 3 or complements thereof. In some embodiments, the targeted portion of the CYP3A5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 408-SEQ ID NO: 411 or complements thereof. In some embodiments, the targeted portion of the CYP3A5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DAB2IP genomic sequence. In some embodiments, the ASO targets a NSAE pre-mRNA transcript from a DAB2IP genomic sequence comprising a NSAE exon. In some embodiments, the ASO targets a NSAE pre-mRNA transcript from a DAB2IP genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000259371.6, premrna_ENST00000309989.1, premrna_ENST00000373782.7, premrna_ENST00000394340.7, premrna_ENST00000408936.7, premrna_ENST00000436835.5, premrna_ENST00000459906.1, premrna_ENST00000465078.1, premrna_ENST00000487716.1, premrna_ENST00000489314.1, premrna_ENST00000648444.1, and premrna_ENST00000648693.1. In some embodiments, the ASO targets a DAB2IP pre-mRNA sequence comprising a NSAE exon. In some embodiments, the ASO targets a DAB2IP pre-mRNA sequence comprising an intron flanking the 3′ splice site of the NSAE exon. In some embodiments, the ASO targets a DAB2IP pre-mRNA sequence comprising an intron flanking the 5′ splice site of the NSAE exon. In some embodiments, the transcript is selected from the group consisting of transcript_ENST00000259371.6, transcript_ENST00000309989.1, transcript_ENST00000373782.7, transcript_ENST00000394340.7, transcript_ENST00000408936.7, transcript_ENST00000436835.5, transcript_ENST00000459906.1, transcript_ENST00000465078.1, transcript_ENST00000487716.1, transcript_ENST00000489314.1, transcript_ENST00000648444.1, and transcript_ENST00000648693.1.

In some embodiments, the DAB2IP pre-mRNA transcript is encoded by a genetic sequence with at least about 80%, 85%, 90%, 95%, 96%, 97%, 98%, 99% or 100% sequence identity to the Ensembl reference number ENSG00000136848.17 or a complement thereof. In some embodiments, the DAB2IP pre-mRNA transcript comprises a sequence with at least about 80%, 85%, 90%, 95%, 96%, 97%, 98%, 99% or 100% sequence identity to a DAB2IP pre-mRNA transcript or a complement thereof described herein.

In some embodiments, the targeted portion of the DAB2IP pre-mRNA comprises a sequence with at least 80%, 85%, 90%, 95%, 97%, or 100% sequence identity to a region comprising at least 8 contiguous nucleic acids of a sequence of premrna_ENST00000259371.6, premrna_ENST00000309989.1, premrna_ENST00000373782.7, premrna_ENST00000394340.7, premrna_ENST00000408936.7, premrna_ENST00000436835.5, premrna_ENST00000459906.1, premrna_ENST00000465078.1, premrna_ENST00000487716.1, premrna_ENST00000489314.1, premrna_ENST00000648444.1, premrna_ENST00000648693.1 or a sequence of Table 3 or complements thereof. In some embodiments, the targeted portion of the DAB2IP pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 412-SEQ ID NO: 415 or complements thereof. In some embodiments, the targeted portion of the DAB2IP pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DBN1 genomic sequence. In some embodiments, the ASO targets a NSAE pre-mRNA transcript from a DBN1 genomic sequence comprising a NSAE exon. In some embodiments, the ASO targets a NSAE pre-mRNA transcript from a DBN1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000292385.9, premrna_ENST00000309007.9, premrna_ENST00000393565.5, premrna_ENST00000467054.2, premrna_ENST00000471767.1, premrna_ENST00000472831.5, premrna_ENST00000477391.6, premrna_ENST00000505550.1, premrna_ENST00000506117.5, premrna_ENST00000512501.1, and premrna_ENST00000514833.1. In some embodiments, the ASO targets a DBN1 pre-mRNA sequence comprising a NSAE exon. In some embodiments, the ASO targets a DBN1 pre-mRNA sequence comprising an intron flanking the 3′ splice site of the NSAE exon. In some embodiments, the ASO targets a DBN1 pre-mRNA sequence comprising an intron flanking the 5′ splice site of the NSAE exon. In some embodiments, the transcript is selected from the group consisting of transcript_ENST00000292385.9, transcript_ENST00000309007.9, transcript_ENST00000393565.5, transcript_ENST00000467054.2, transcript_ENST00000471767.1, transcript_ENST00000472831.5, transcript_ENST00000477391.6, transcript_ENST00000505550.1, transcript_ENST00000506117.5, transcript_ENST00000512501.1, and transcript_ENST00000514833.1.

In some embodiments, the DBN1 pre-mRNA transcript is encoded by a genetic sequence with at least about 80%, 85%, 90%, 95%, 96%, 97%, 98%, 99% or 100% sequence identity to the Ensembl reference number ENSG00000113758.13 or a complement thereof. In some embodiments, the DBN1 pre-mRNA transcript comprises a sequence with at least about 80%, 85%, 90%, 95%, 96%, 97%, 98%, 99% or 100% sequence identity to a DBN1 pre-mRNA transcript or a complement thereof described herein.

In some embodiments, the targeted portion of the DBN1 pre-mRNA comprises a sequence with at least 80%, 85%, 90%, 95%, 97%, or 100% sequence identity to a region comprising at least 8 contiguous nucleic acids of a sequence of premrna_ENST00000292385.9, premrna_ENST00000309007.9, premrna_ENST00000393565.5, premrna_ENST00000467054.2, premrna_ENST00000471767.1, premrna_ENST00000472831.5, premrna_ENST00000477391.6, premrna_ENST00000505550.1, premrna_ENST00000506117.5, premrna_ENST00000512501.1, premrna_ENST00000514833.1 or a sequence of Table 3 or complements thereof. In some embodiments, the targeted portion of the DBN1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 416-SEQ ID NO: 424 or complements thereof. In some embodiments, the targeted portion of the DBN1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DDX51 genomic sequence. In some embodiments, the ASO targets a NSAE pre-mRNA transcript from a DDX51 genomic sequence comprising a NSAE exon. In some embodiments, the ASO targets a NSAE pre-mRNA transcript from a DDX51 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000329073.8, premrna_ENST00000397333.4, premrna_ENST00000462829.2, premrna_ENST00000541489.5, premrna_ENST00000545991.1, and premrna_ENST00000546058.1. In some embodiments, the ASO targets a DDX51 pre-mRNA sequence comprising a NSAE exon. In some embodiments, the ASO targets a DDX51 pre-mRNA sequence comprising an intron flanking the 3′ splice site of the NSAE exon. In some embodiments, the ASO targets a DDX51 pre-mRNA sequence comprising an intron flanking the 5′ splice site of the NSAE exon. In some embodiments, the transcript is selected from the group consisting of transcript_ENST00000329073.8, transcript_ENST00000397333.4, transcript_ENST00000462829.2, transcript_ENST00000541489.5, transcript_ENST00000545991.1, and transcript_ENST00000546058.1.

In some embodiments, the DDX51 pre-mRNA transcript is encoded by a genetic sequence with at least about 80%, 85%, 90%, 95%, 96%, 97%, 98%, 99% or 100% sequence identity to the Ensembl reference number ENSG00000185163.10 or ENSG00000288503.1 or a complement thereof. In some embodiments, the DDX51 pre-mRNA transcript comprises a sequence with at least about 80%, 85%, 90%, 95%, 96%, 97%, 98%, 99% or 100% sequence identity to a DDX51 pre-mRNA transcript or a complement thereof described herein.

In some embodiments, the targeted portion of the DDX51 pre-mRNA comprises a sequence with at least 80%, 85%, 90%, 95%, 97%, or 100% sequence identity to a region comprising at least 8 contiguous nucleic acids of a sequence of premrna_ENST00000329073.8, premrna_ENST00000397333.4, premrna_ENST00000462829.2, premrna_ENST00000541489.5, premrna_ENST00000545991.1, premrna_ENST00000546058.1 or a sequence of Table 3 or complements thereof. In some embodiments, the targeted portion of the DDX51 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 425-SEQ ID NO: 428 or complements thereof. In some embodiments, the targeted portion of the DDX51 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DEAF1 genomic sequence. In some embodiments, the ASO targets a NSAE pre-mRNA transcript from a DEAF1 genomic sequence comprising a NSAE exon. In some embodiments, the ASO targets a NSAE pre-mRNA transcript from a DEAF1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000382409.3, premrna_ENST00000524786.1, premrna_ENST00000525626.5, premrna_ENST00000525904.5, premrna_ENST00000526790.1, premrna_ENST00000526857.2, premrna_ENST00000527170.5, premrna_ENST00000527658.1, premrna_ENST00000528864.5, premrna_ENST00000529717.5, premrna_ENST00000529727.1, and premrna_ENST00000530813.1. In some embodiments, the ASO targets a DEAF1 pre-mRNA sequence comprising a NSAE exon. In some embodiments, the ASO targets a DEAF1 pre-mRNA sequence comprising an intron flanking the 3′ splice site of the NSAE exon. In some embodiments, the ASO targets a DEAF1 pre-mRNA sequence comprising an intron flanking the 5′ splice site of the NSAE exon. In some embodiments, the transcript is selected from the group consisting of transcript_ENST00000382409.3, transcript_ENST00000524786.1, transcript_ENST00000525626.5, transcript_ENST00000525904.5, transcript_ENST00000526790.1, transcript_ENST00000526857.2, transcript_ENST00000527170.5, transcript_ENST00000527658.1, transcript_ENST00000528864.5, transcript_ENST00000529717.5, transcript_ENST00000529727.1, and transcript_ENST00000530813.1.

In some embodiments, the DEAF1 pre-mRNA transcript is encoded by a genetic sequence with at least about 80%, 85%, 90%, 95%, 96%, 97%, 98%, 99% or 100% sequence identity to the Ensembl reference number ENSG00000177030.17 or ENSG00000282712.1 a complement thereof. In some embodiments, the DEAF1 pre-mRNA transcript comprises a sequence with at least about 80%, 85%, 90%, 95%, 96%, 97%, 98%, 99% or 100% sequence identity to a DEAF1 pre-mRNA transcript or a complement thereof described herein.

In some embodiments, the targeted portion of the DEAF1 pre-mRNA comprises a sequence with at least 80%, 85%, 90%, 95%, 97%, or 100% sequence identity to a region comprising at least 8 contiguous nucleic acids of a sequence of premrna_ENST00000382409.3, premrna_ENST00000524786.1, premrna_ENST00000525626.5, premrna_ENST00000525904.5, premrna_ENST00000526790.1, premrna_ENST00000526857.2, premrna_ENST00000527170.5, premrna_ENST00000527658.1, premrna_ENST00000528864.5, premrna_ENST00000529717.5, premrna_ENST00000529727.1, premrna_ENST00000530813.1 or a sequence of Table 3 or complements thereof. In some embodiments, the targeted portion of the DEAF1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 429-SEQ ID NO: 432 or complements thereof. In some embodiments, the targeted portion of the DEAF1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DGKE genomic sequence. In some embodiments, the ASO targets a NSAE pre-mRNA transcript from a DGKE genomic sequence comprising a NSAE exon. In some embodiments, the ASO targets a NSAE pre-mRNA transcript from a DGKE genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000284061.8, premrna_ENST00000570738.1, premrna_ENST00000571084.1, premrna_ENST00000572810.1, premrna_ENST00000572944.1, and premrna_ENST00000576869.5. In some embodiments, the ASO targets a DGKE pre-mRNA sequence comprising a NSAE exon. In some embodiments, the ASO targets a DGKE pre-mRNA sequence comprising an intron flanking the 3′ splice site of the NSAE exon. In some embodiments, the ASO targets a DGKE pre-mRNA sequence comprising an intron flanking the 5′ splice site of the NSAE exon. In some embodiments, the transcript is selected from the group consisting of transcript_ENST00000284061.8, transcript_ENST00000570738.1, transcript_ENST00000571084.1, transcript_ENST00000572810.1, transcript_ENST00000572944.1, and transcript_ENST00000576869.5.

In some embodiments, the DGKE pre-mRNA transcript is encoded by a genetic sequence with at least about 80%, 85%, 90%, 95%, 96%, 97%, 98%, 99% or 100% sequence identity to the Ensembl reference number ENSG00000153933.10 or a complement thereof. In some embodiments, the DGKE pre-mRNA transcript comprises a sequence with at least about 80%, 85%, 90%, 95%, 96%, 97%, 98%, 99% or 100% sequence identity to a DGKE pre-mRNA transcript or a complement thereof described herein.

In some embodiments, the targeted portion of the DGKE pre-mRNA comprises a sequence with at least 80%, 85%, 90%, 95%, 97%, or 100% sequence identity to a region comprising at least 8 contiguous nucleic acids of a sequence of premrna_ENST00000284061.8, premrna_ENST00000570738.1, premrna_ENST00000571084.1, premrna_ENST00000572810.1, premrna_ENST00000572944.1, premrna_ENST00000576869.5 or a sequence of Table 3 or complements thereof. In some embodiments, the targeted portion of the DGKE pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 433-SEQ ID NO: 436 or complements thereof. In some embodiments, the targeted portion of the DGKE pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DHDDS genomic sequence. In some embodiments, the ASO targets a NSAE pre-mRNA transcript from a DHDDS genomic sequence comprising a NSAE exon. In some embodiments, the ASO targets a NSAE pre-mRNA transcript from a DHDDS genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000236342.12, premrna_ENST00000360009.6, premrna_ENST00000374185.7, premrna_ENST00000374186.7, premrna_ENST00000416052.1, premrna_ENST00000427245.6, premrna_ENST00000430232.5, premrna_ENST00000431933.5, premrna_ENST00000434391.6, premrna_ENST00000436153.6, premrna_ENST00000487944.5, premrna_ENST00000525165.5, premrna_ENST00000525326.5, premrna_ENST00000525410.1, premrna_ENST00000525546.5, premrna_ENST00000525682.6, premrna_ENST00000526219.5, premrna_ENST00000526278.5, premrna_ENST00000527611.1, premrna_ENST00000528557.5, premrna_ENST00000529688.5, premrna_ENST00000530781.5, premrna_ENST00000531312.5, premrna_ENST00000531955.5, and premrna_ENST00000533087.5. In some embodiments, the ASO targets a DHDDS pre-mRNA sequence comprising a NSAE exon. In some embodiments, the ASO targets a DHDDS pre-mRNA sequence comprising an intron flanking the 3′ splice site of the NSAE exon. In some embodiments, the ASO targets a DHDDS pre-mRNA sequence comprising an intron flanking the 5′ splice site of the NSAE exon. In some embodiments, the transcript is selected from the group consisting of transcript_ENST00000236342.12, transcript_ENST00000360009.6, transcript_ENST00000374185.7, transcript_ENST00000374186.7, transcript_ENST00000416052.1, transcript_ENST00000427245.6, transcript_ENST00000430232.5, transcript_ENST00000431933.5, transcript_ENST00000434391.6, transcript_ENST00000436153.6, transcript_ENST00000487944.5, transcript_ENST00000525165.5, transcript_ENST00000525326.5, transcript_ENST00000525410.1, transcript_ENST00000525546.5, transcript_ENST00000525682.6, transcript_ENST00000526219.5, transcript_ENST00000526278.5, transcript_ENST00000527611.1, transcript_ENST00000528557.5, transcript_ENST00000529688.5, transcript_ENST00000530781.5, transcript_ENST00000531312.5, transcript_ENST00000531955.5, and transcript_ENST00000533087.5.

In some embodiments, the DHDDS pre-mRNA transcript is encoded by a genetic sequence with at least about 80%, 85%, 90%, 95%, 96%, 97%, 98%, 99% or 100% sequence identity to the Ensembl reference number ENSG00000117682.17 or a complement thereof. In some embodiments, the DHDDS pre-mRNA transcript comprises a sequence with at least about 80%, 85%, 90%, 95%, 96%, 97%, 98%, 99% or 100% sequence identity to a DHDDS pre-mRNA transcript or a complement thereof described herein.

In some embodiments, the targeted portion of the DHDDS pre-mRNA comprises a sequence with at least 80%, 85%, 90%, 95%, 97%, or 100% sequence identity to a region comprising at least 8 contiguous nucleic acids of a sequence of premrna_ENST00000236342.12, premrna_ENST00000360009.6, premrna_ENST00000374185.7, premrna_ENST00000374186.7, premrna_ENST00000416052.1, premrna_ENST00000427245.6, premrna_ENST00000430232.5, premrna_ENST00000431933.5, premrna_ENST00000434391.6, premrna_ENST00000436153.6, premrna_ENST00000487944.5, premrna_ENST00000525165.5, premrna_ENST00000525326.5, premrna_ENST00000525410.1, premrna_ENST00000525546.5, premrna_ENST00000525682.6, premrna_ENST00000526219.5, premrna_ENST00000526278.5, premrna_ENST00000527611.1, premrna_ENST00000528557.5, premrna_ENST00000529688.5, premrna_ENST00000530781.5, premrna_ENST00000531312.5, premrna_ENST00000531955.5, premrna_ENST00000533087.5, or a sequence of Table 3 or complements thereof. In some embodiments, the targeted portion of the DHDDS pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 437-SEQ ID NO: 444 or complements thereof. In some embodiments, the targeted portion of the DHDDS pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DMKN genomic sequence. In some embodiments, the ASO targets a NSAE pre-mRNA transcript from a DMKN genomic sequence comprising a NSAE exon. In some embodiments, the ASO targets a NSAE pre-mRNA transcript from a DMKN genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000339686.8, premrna_ENST00000392206.6, premrna_ENST00000402589.6, premrna_ENST00000408915.6, premrna_ENST00000414866.6, premrna_ENST00000418261.5, premrna_ENST00000419602.5, premrna_ENST00000424570.6, premrna_ENST00000429837.5, premrna_ENST00000434389.5, premrna_ENST00000436012.5, premrna_ENST00000443640.5, premrna_ENST00000447113.6, premrna_ENST00000450261.1, premrna_ENST00000451297.6, premrna_ENST00000458071.5, premrna_ENST00000460051.5, premrna_ENST00000460327.5, premrna_ENST00000461300.5, premrna_ENST00000462126.5, premrna_ENST00000462538.5, premrna_ENST00000462721.5, premrna_ENST00000463292.5, premrna_ENST00000464709.6, premrna_ENST00000464894.5, premrna_ENST00000465927.5, premrna_ENST00000466587.6, premrna_ENST00000467532.5, premrna_ENST00000467637.5, premrna_ENST00000469960.5, premrna_ENST00000470746.6, premrna_ENST00000470857.1, premrna_ENST00000470964.1, premrna_ENST00000471017.5, premrna_ENST00000471786.6, premrna_ENST00000472065.1, premrna_ENST00000472252.6, premrna_ENST00000472365.6, premrna_ENST00000474928.5, premrna_ENST00000474992.5, premrna_ENST00000476051.5, premrna_ENST00000476246.5, premrna_ENST00000478648.6, premrna_ENST00000480502.5, premrna_ENST00000480507.1, premrna_ENST00000482321.5, premrna_ENST00000483855.5, premrna_ENST00000484064.5, premrna_ENST00000486450.5, premrna_ENST00000486756.6, premrna_ENST00000488542.6, premrna_ENST00000488762.5, premrna_ENST00000488892.5, premrna_ENST00000489395.6, premrna_ENST00000490622.5, premrna_ENST00000492341.6, premrna_ENST00000493517.5, premrna_ENST00000493979.5, premrna_ENST00000498211.5, premrna_ENST00000498269.5, premrna_ENST00000498593.5, premrna_ENST00000593342.5, premrna_ENST00000595571.5, premrna_ENST00000597212.5, premrna_ENST00000601368.5, premrna_ENST00000601650.1, premrna_ENST00000602679.5, and premrna_ENST00000602781.5. In some embodiments, the ASO targets a DMKN pre-mRNA sequence comprising a NSAE exon. In some embodiments, the ASO targets a DMKN pre-mRNA sequence comprising an intron flanking the 3′ splice site of the NSAE exon. In some embodiments, the ASO targets a DMKN pre-mRNA sequence comprising an intron flanking the 5′ splice site of the NSAE exon. In some embodiments, the transcript is selected from the group consisting of transcript_ENST00000339686.8, transcript_ENST00000392206.6, transcript_ENST00000402589.6, transcript_ENST00000408915.6, transcript_ENST00000414866.6, transcript_ENST00000418261.5, transcript_ENST00000419602.5, transcript_ENST00000424570.6, transcript_ENST00000429837.5, transcript_ENST00000434389.5, transcript_ENST00000436012.5, transcript_ENST00000443640.5, transcript_ENST00000447113.6, transcript_ENST00000450261.1, transcript_ENST00000451297.6, transcript_ENST00000458071.5, transcript_ENST00000460051.5, transcript_ENST00000460327.5, transcript_ENST00000461300.5, transcript_ENST00000462126.5, transcript_ENST00000462538.5, transcript_ENST00000462721.5, transcript_ENST00000463292.5, transcript_ENST00000464709.6, transcript_ENST00000464894.5, transcript_ENST00000465927.5, transcript_ENST00000466587.6, transcript_ENST00000467532.5, transcript_ENST00000467637.5, transcript_ENST00000469960.5, transcript_ENST00000470746.6, transcript_ENST00000470857.1, transcript_ENST00000470964.1, transcript_ENST00000471017.5, transcript_ENST00000471786.6, transcript_ENST00000472065.1, transcript_ENST00000472252.6, transcript_ENST00000472365.6, transcript_ENST00000474928.5, transcript_ENST00000474992.5, transcript_ENST00000476051.5, transcript_ENST00000476246.5, transcript_ENST00000478648.6, transcript_ENST00000480502.5, transcript_ENST00000480507.1, transcript_ENST00000482321.5, transcript_ENST00000483855.5, transcript_ENST00000484064.5, transcript_ENST00000486450.5, transcript_ENST00000486756.6, transcript_ENST00000488542.6, transcript_ENST00000488762.5, transcript_ENST00000488892.5, transcript_ENST00000489395.6, transcript_ENST00000490622.5, transcript_ENST00000492341.6, transcript_ENST00000493517.5, transcript_ENST00000493979.5, transcript_ENST00000498211.5, transcript_ENST00000498269.5, transcript_ENST00000498593.5, transcript_ENST00000593342.5, transcript_ENST00000595571.5, transcript_ENST00000597212.5, transcript_ENST00000601368.5, transcript_ENST00000601650.1, transcript_ENST00000602679.5, and transcript_ENST00000602781.5.

In some embodiments, the DMKN pre-mRNA transcript is encoded by a genetic sequence with at least about 80%, 85%, 90%, 95%, 96%, 97%, 98%, 99% or 100% sequence identity to the Ensembl reference number ENSG00000161249.21 or a complement thereof. In some embodiments, the DMKN pre-mRNA transcript comprises a sequence with at least about 80%, 85%, 90%, 95%, 96%, 97%, 98%, 99% or 100% sequence identity to a DMKN pre-mRNA transcript or a complement thereof described herein.

In some embodiments, the targeted portion of the DMKN pre-mRNA comprises a sequence with at least 80%, 85%, 90%, 95%, 97%, or 100% sequence identity to a region comprising at least 8 contiguous nucleic acids of a sequence of premrna_ENST00000339686.8, premrna_ENST00000392206.6, premrna_ENST00000402589.6, premrna_ENST00000408915.6, premrna_ENST00000414866.6, premrna_ENST00000418261.5, premrna_ENST00000419602.5, premrna_ENST00000424570.6, premrna_ENST00000429837.5, premrna_ENST00000434389.5, premrna_ENST00000436012.5, premrna_ENST00000443640.5, premrna_ENST00000447113.6, premrna_ENST00000450261.1, premrna_ENST00000451297.6, premrna_ENST00000458071.5, premrna_ENST00000460051.5, premrna_ENST00000460327.5, premrna_ENST00000461300.5, premrna_ENST00000462126.5, premrna_ENST00000462538.5, premrna_ENST00000462721.5, premrna_ENST00000463292.5, premrna_ENST00000464709.6, premrna_ENST00000464894.5, premrna_ENST00000465927.5, premrna_ENST00000466587.6, premrna_ENST00000467532.5, premrna_ENST00000467637.5, premrna_ENST00000469960.5, premrna_ENST00000470746.6, premrna_ENST00000470857.1, premrna_ENST00000470964.1, premrna_ENST00000471017.5, premrna_ENST00000471786.6, premrna_ENST00000472065.1, premrna_ENST00000472252.6, premrna_ENST00000472365.6, premrna_ENST00000474928.5, premrna_ENST00000474992.5, premrna_ENST00000476051.5, premrna_ENST00000476246.5, premrna_ENST00000478648.6, premrna_ENST00000480502.5, premrna_ENST00000480507.1, premrna_ENST00000482321.5, premrna_ENST00000483855.5, premrna_ENST00000484064.5, premrna_ENST00000486450.5, premrna_ENST00000486756.6, premrna_ENST00000488542.6, premrna_ENST00000488762.5, premrna_ENST00000488892.5, premrna_ENST00000489395.6, premrna_ENST00000490622.5, premrna_ENST00000492341.6, premrna_ENST00000493517.5, premrna_ENST00000493979.5, premrna_ENST00000498211.5, premrna_ENST00000498269.5, premrna_ENST00000498593.5, premrna_ENST00000593342.5, premrna_ENST00000595571.5, premrna_ENST00000597212.5, premrna_ENST00000601368.5, premrna_ENST00000601650.1, premrna_ENST00000602679.5, premrna_ENST00000602781.5 or a sequence of Table 3 or complements thereof. In some embodiments, the targeted portion of the DMKN pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 445-SEQ ID NO: 448 or complements thereof. In some embodiments, the targeted portion of the DMKN pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DNHD1 genomic sequence. In some embodiments, the ASO targets a NSAE pre-mRNA transcript from a DNHD1 genomic sequence comprising a NSAE exon. In some embodiments, the ASO targets a NSAE pre-mRNA transcript from a DNHD1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000254579.11, premrna_ENST00000354685.7, premrna_ENST00000472080.1, premrna_ENST00000473019.5, premrna_ENST00000477562.1, premrna_ENST00000524401.2, premrna_ENST00000525080.1, premrna_ENST00000525883.5, premrna_ENST00000526027.1, premrna_ENST00000527143.1, premrna_ENST00000529821.5, premrna_ENST00000530197.5, premrna_ENST00000531903.5, premrna_ENST00000532027.5, premrna_ENST00000532467.1, premrna_ENST00000533635.5, premrna_ENST00000533649.1, and premrna_ENST00000534210.6. In some embodiments, the ASO targets a DNHD1 pre-mRNA sequence comprising a NSAE exon. In some embodiments, the ASO targets a DNHD1 pre-mRNA sequence comprising an intron flanking the 3′ splice site of the NSAE exon. In some embodiments, the ASO targets a DNHD1 pre-mRNA sequence comprising an intron flanking the 5′ splice site of the NSAE exon. In some embodiments, the transcript is selected from the group consisting of transcript_ENST00000254579.11, transcript_ENST00000354685.7, transcript_ENST00000472080.1, transcript_ENST00000473019.5, transcript_ENST00000477562.1, transcript_ENST00000524401.2, transcript_ENST00000525080.1, transcript_ENST00000525883.5, transcript_ENST00000526027.1, transcript_ENST00000527143.1, transcript_ENST00000529821.5, transcript_ENST00000530197.5, transcript_ENST00000531903.5, transcript_ENST00000532027.5, transcript_ENST00000532467.1, transcript_ENST00000533635.5, transcript_ENST00000533649.1, and transcript_ENST00000534210.6.

In some embodiments, the DNHD1 pre-mRNA transcript is encoded by a genetic sequence with at least about 80%, 85%, 90%, 95%, 96%, 97%, 98%, 99% or 100% sequence identity to the Ensembl reference number ENSG00000179532.13 or a complement thereof. In some embodiments, the DNHD1 pre-mRNA transcript comprises a sequence with at least about 80%, 85%, 90%, 95%, 96%, 97%, 98%, 99% or 100% sequence identity to a DNHD1 pre-mRNA transcript or a complement thereof described herein.

In some embodiments, the targeted portion of the DNHD1 pre-mRNA comprises a sequence with at least 80%, 85%, 90%, 95%, 97%, or 100% sequence identity to a region comprising at least 8 contiguous nucleic acids of a sequence of premrna_ENST00000254579.11, premrna_ENST00000354685.7, premrna_ENST00000472080.1, premrna_ENST00000473019.5, premrna_ENST00000477562.1, premrna_ENST00000524401.2, premrna_ENST00000525080.1, premrna_ENST00000525883.5, premrna_ENST00000526027.1, premrna_ENST00000527143.1, premrna_ENST00000529821.5, premrna_ENST00000530197.5, premrna_ENST00000531903.5, premrna_ENST00000532027.5, premrna_ENST00000532467.1, premrna_ENST00000533635.5, premrna_ENST00000533649.1, premrna_ENST00000534210.6 or a sequence of Table 3 or complements thereof. In some embodiments, the targeted portion of the DNHD1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 449-SEQ ID NO: 452 or complements thereof. In some embodiments, the targeted portion of the DNHD1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DOCK8 genomic sequence. In some embodiments, the ASO targets a NSAE pre-mRNA transcript from a DOCK8 genomic sequence comprising a NSAE exon. In some embodiments, the ASO targets a NSAE pre-mRNA transcript from a DOCK8 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000382329.1, premrna_ENST00000382331.5, premrna_ENST00000382341.5, premrna_ENST00000432829.6, premrna_ENST00000453981.5, premrna_ENST00000454469.6, premrna_ENST00000462618.1, premrna_ENST00000469197.5, premrna_ENST00000469391.5, premrna_ENST00000474772.1, premrna_ENST00000478380.5, premrna_ENST00000479404.5, premrna_ENST00000483757.5, premrna_ENST00000487230.5, premrna_ENST00000493666.2, premrna_ENST00000495184.5, and premrna_ENST00000524396.5. In some embodiments, the ASO targets a DOCK8 pre-mRNA sequence comprising a NSAE exon. In some embodiments, the ASO targets a DOCK8 pre-mRNA sequence comprising an intron flanking the 3′ splice site of the NSAE exon. In some embodiments, the ASO targets a DOCK8 pre-mRNA sequence comprising an intron flanking the 5′ splice site of the NSAE exon. In some embodiments, the transcript is selected from the group consisting of transcript_ENST00000382329.1, transcript_ENST00000382331.5, transcript_ENST00000382341.5, transcript_ENST00000432829.6, transcript_ENST00000453981.5, transcript_ENST00000454469.6, transcript_ENST00000462618.1, transcript_ENST00000469197.5, transcript_ENST00000469391.5, transcript_ENST00000474772.1, transcript_ENST00000478380.5, transcript_ENST00000479404.5, transcript_ENST00000483757.5, transcript_ENST00000487230.5, transcript_ENST00000493666.2, transcript_ENST00000495184.5, and transcript_ENST00000524396.5.

In some embodiments, the DOCK8 pre-mRNA transcript is encoded by a genetic sequence with at least about 80%, 85%, 90%, 95%, 96%, 97%, 98%, 99% or 100% sequence identity to the Ensembl reference number ENSG00000107099.15 or a complement thereof. In some embodiments, the DOCK8 pre-mRNA transcript comprises a sequence with at least about 80%, 85%, 90%, 95%, 96%, 97%, 98%, 99% or 100% sequence identity to a DOCK8 pre-mRNA transcript or a complement thereof described herein.

In some embodiments, the targeted portion of the DOCK8 pre-mRNA comprises a sequence with at least 80%, 85%, 90%, 95%, 97%, or 100% sequence identity to a region comprising at least 8 contiguous nucleic acids of a sequence of premrna_ENST00000382329.1, premrna_ENST00000382331.5, premrna_ENST00000382341.5, premrna_ENST00000432829.6, premrna_ENST00000453981.5, premrna_ENST00000454469.6, premrna_ENST00000462618.1, premrna_ENST00000469197.5, premrna_ENST00000469391.5, premrna_ENST00000474772.1, premrna_ENST00000478380.5, premrna_ENST00000479404.5, premrna_ENST00000483757.5, premrna_ENST00000487230.5, premrna_ENST00000493666.2, premrna_ENST00000495184.5, premrna_ENST00000524396.5 or a sequence of Table 3 or complements thereof. In some embodiments, the targeted portion of the DOCK8 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 453-SEQ ID NO: 456 or complements thereof. In some embodiments, the targeted portion of the DOCK8 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a DOK5 genomic sequence. In some embodiments, the ASO targets a NSAE pre-mRNA transcript from a DOK5 genomic sequence comprising a NSAE exon. In some embodiments, the ASO targets a NSAE pre-mRNA transcript from a DOK5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000262593.10, premrna_ENST00000395939.5, premrna_ENST00000484860.1, and premrna_ENST00000491469.1. In some embodiments, the ASO targets a DOK5 pre-mRNA sequence comprising a NSAE exon. In some embodiments, the ASO targets a DOK5 pre-mRNA sequence comprising an intron flanking the 3′ splice site of the NSAE exon. In some embodiments, the ASO targets a DOK5 pre-mRNA sequence comprising an intron flanking the 5′ splice site of the NSAE exon. In some embodiments, the transcript is selected from the group consisting of transcript_ENST00000262593.10, transcript_ENST00000395939.5, transcript_ENST00000484860.1, and transcript_ENST00000491469.1.

In some embodiments, the DOK5 pre-mRNA transcript is encoded by a genetic sequence with at least about 80%, 85%, 90%, 95%, 96%, 97%, 98%, 99% or 100% sequence identity to the Ensembl reference number ENSG00000101134.12 or a complement thereof. In some embodiments, the DOK5 pre-mRNA transcript comprises a sequence with at least about 80%, 85%, 90%, 95%, 96%, 97%, 98%, 99% or 100% sequence identity to a DOK5 pre-mRNA transcript or a complement thereof described herein.

In some embodiments, the targeted portion of the DOK5 pre-mRNA comprises a sequence with at least 80%, 85%, 90%, 95%, 97%, or 100% sequence identity to a region comprising at least 8 contiguous nucleic acids of a sequence of premrna_ENST00000262593.10, premrna_ENST00000395939.5, premrna_ENST00000484860.1, premrna_ENST00000491469.1 or a sequence of Table 3 or complements thereof. In some embodiments, the targeted portion of the DOK5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 457-SEQ ID NO: 460 or complements thereof. In some embodiments, the targeted portion of the DOK5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a EIF2B5 genomic sequence. In some embodiments, the ASO targets a NSAE pre-mRNA transcript from a EIF2B5 genomic sequence comprising a NSAE exon. In some embodiments, the ASO targets a NSAE pre-mRNA transcript from a EIF2B5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000432569.2, premrna_ENST00000432982.5, premrna_ENST00000465218.3, premrna_ENST00000468748.7, premrna_ENST00000471832.2, premrna_ENST00000479250.1, premrna_ENST00000481054.5, premrna_ENST00000484154.2, premrna_ENST00000491008.6, premrna_ENST00000491144.5, premrna_ENST00000492226.2, premrna_ENST00000492773.6, premrna_ENST00000493740.1, premrna_ENST00000498831.1, premrna_ENST00000647636.1, premrna_ENST00000647909.1, premrna_ENST00000648145.1, premrna_ENST00000648189.1, premrna_ENST00000648256.1, premrna_ENST00000648314.1, premrna_ENST00000648599.1, premrna_ENST00000648630.1, premrna_ENST00000648682.1, premrna_ENST00000648882.1, premrna_ENST00000648890.1, premrna_ENST00000648915.2, premrna_ENST00000649545.1, premrna_ENST00000649688.1, premrna_ENST00000649814.1, premrna_ENST00000650244.1, and premrna_ENST00000650270.1. In some embodiments, the ASO targets a EIF2B5 pre-mRNA sequence comprising a NSAE exon. In some embodiments, the ASO targets a EIF2B5 pre-mRNA sequence comprising an intron flanking the 3′ splice site of the NSAE exon. In some embodiments, the ASO targets a EIF2B5 pre-mRNA sequence comprising an intron flanking the 5′ splice site of the NSAE exon. In some embodiments, the transcript is selected from the group consisting of transcript_ENST00000432569.2, transcript_ENST00000432982.5, transcript_ENST00000465218.3, transcript_ENST00000468748.7, transcript_ENST00000471832.2, transcript_ENST00000479250.1, transcript_ENST00000481054.5, transcript_ENST00000484154.2, transcript_ENST00000491008.6, transcript_ENST00000491144.5, transcript_ENST00000492226.2, transcript_ENST00000492773.6, transcript_ENST00000493740.1, transcript_ENST00000498831.1, transcript_ENST00000647636.1, transcript_ENST00000647909.1, transcript_ENST00000648145.1, transcript_ENST00000648189.1, transcript_ENST00000648256.1, transcript_ENST00000648314.1, transcript_ENST00000648599.1, transcript_ENST00000648630.1, transcript_ENST00000648682.1, transcript_ENST00000648882.1, transcript_ENST00000648890.1, transcript_ENST00000648915.2, transcript_ENST00000649545.1, transcript_ENST00000649688.1, transcript_ENST00000649814.1, transcript_ENST00000650244.1, and transcript_ENST00000650270.1.

In some embodiments, the EIF2B5 pre-mRNA transcript is encoded by a genetic sequence with at least about 80%, 85%, 90%, 95%, 96%, 97%, 98%, 99% or 100% sequence identity to the Ensembl reference number ENSG00000145191.15 or a complement thereof. In some embodiments, the EIF2B5 pre-mRNA transcript comprises a sequence with at least about 80%, 85%, 90%, 95%, 96%, 97%, 98%, 99% or 100% sequence identity to a EIF2B5 pre-mRNA transcript or a complement thereof described herein.

In some embodiments, the targeted portion of the EIF2B5 pre-mRNA comprises a sequence with at least 80%, 85%, 90%, 95%, 97%, or 100% sequence identity to a region comprising at least 8 contiguous nucleic acids of a sequence of premrna_ENST00000432569.2, premrna_ENST00000432982.5, premrna_ENST00000465218.3, premrna_ENST00000468748.7, premrna_ENST00000471832.2, premrna_ENST00000479250.1, premrna_ENST00000481054.5, premrna_ENST00000484154.2, premrna_ENST00000491008.6, premrna_ENST00000491144.5, premrna_ENST00000492226.2, premrna_ENST00000492773.6, premrna_ENST00000493740.1, premrna_ENST00000498831.1, premrna_ENST00000647636.1, premrna_ENST00000647909.1, premrna_ENST00000648145.1, premrna_ENST00000648189.1, premrna_ENST00000648256.1, premrna_ENST00000648314.1, premrna_ENST00000648599.1, premrna_ENST00000648630.1, premrna_ENST00000648682.1, premrna_ENST00000648882.1, premrna_ENST00000648890.1, premrna_ENST00000648915.2, premrna_ENST00000649545.1, premrna_ENST00000649688.1, premrna_ENST00000649814.1, premrna_ENST00000650244.1, premrna_ENST00000650270.1 or a sequence of Table 3 or complements thereof. In some embodiments, the targeted portion of the EIF2B5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 461-SEQ ID NO: 464 or complements thereof. In some embodiments, the targeted portion of the EIF2B5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ELAC2 genomic sequence. In some embodiments, the ASO targets a NSAE pre-mRNA transcript from a ELAC2 genomic sequence comprising a NSAE exon. In some embodiments, the ASO targets a NSAE pre-mRNA transcript from a ELAC2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000338034.9, premrna_ENST00000395962.6, premrna_ENST00000426905.7, premrna_ENST00000446899.5, premrna_ENST00000465825.5, premrna_ENST00000476042.1, premrna_ENST00000480891.5, premrna_ENST00000484122.5, premrna_ENST00000487229.6, premrna_ENST00000491478.5, premrna_ENST00000492559.5, premrna_ENST00000578071.1, premrna_ENST00000578104.1, premrna_ENST00000578991.5, premrna_ENST00000580504.5, premrna_ENST00000581499.6, premrna_ENST00000583371.5, premrna_ENST00000584650.5, premrna_ENST00000609101.5, premrna_ENST00000609345.1, and premrna_ENST00000609757.5. In some embodiments, the ASO targets a ELAC2 pre-mRNA sequence comprising a NSAE exon. In some embodiments, the ASO targets a ELAC2 pre-mRNA sequence comprising an intron flanking the 3′ splice site of the NSAE exon. In some embodiments, the ASO targets a ELAC2 pre-mRNA sequence comprising an intron flanking the 5′ splice site of the NSAE exon. In some embodiments, the transcript is selected from the group consisting of transcript_ENST00000338034.9, transcript_ENST00000395962.6, transcript_ENST00000426905.7, transcript_ENST00000446899.5, transcript_ENST00000465825.5, transcript_ENST00000476042.1, transcript_ENST00000480891.5, transcript_ENST00000484122.5, transcript_ENST00000487229.6, transcript_ENST00000491478.5, transcript_ENST00000492559.5, transcript_ENST00000578071.1, transcript_ENST00000578104.1, transcript_ENST00000578991.5, transcript_ENST00000580504.5, transcript_ENST00000581499.6, transcript_ENST00000583371.5, transcript_ENST00000584650.5, transcript_ENST00000609101.5, transcript_ENST00000609345.1, and transcript_ENST00000609757.5.

In some embodiments, the ELAC2 pre-mRNA transcript is encoded by a genetic sequence with at least about 80%, 85%, 90%, 95%, 96%, 97%, 98%, 99% or 100% sequence identity to the Ensembl reference number ENSG00000006744.19 or a complement thereof. In some embodiments, the ELAC2 pre-mRNA transcript comprises a sequence with at least about 80%, 85%, 90%, 95%, 96%, 97%, 98%, 99% or 100% sequence identity to a ELAC2 pre-mRNA transcript or a complement thereof described herein.

In some embodiments, the targeted portion of the ELAC2 pre-mRNA comprises a sequence with at least 80%, 85%, 90%, 95%, 97%, or 100% sequence identity to a region comprising at least 8 contiguous nucleic acids of a sequence of premrna_ENST00000338034.9, premrna_ENST00000395962.6, premrna_ENST00000426905.7, premrna_ENST00000446899.5, premrna_ENST00000465825.5, premrna_ENST00000476042.1, premrna_ENST00000480891.5, premrna_ENST00000484122.5, premrna_ENST00000487229.6, premrna_ENST00000491478.5, premrna_ENST00000492559.5, premrna_ENST00000578071.1, premrna_ENST00000578104.1, premrna_ENST00000578991.5, premrna_ENST00000580504.5, premrna_ENST00000581499.6, premrna_ENST00000583371.5, premrna_ENST00000584650.5, premrna_ENST00000609101.5, premrna_ENST00000609345.1, premrna_ENST00000609757.5 or a sequence of Table 3 or complements thereof. In some embodiments, the targeted portion of the ELAC2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 465-SEQ ID NO: 472 or complements thereof. In some embodiments, the targeted portion of the ELAC2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ELP1 genomic sequence. In some embodiments, the ASO targets a NSAE pre-mRNA transcript from a ELP1 genomic sequence comprising a NSAE exon. In some embodiments, the ASO targets a NSAE pre-mRNA transcript from a ELP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000374647.10, premrna_ENST00000467959.1, premrna_ENST00000495759.5, and premrna_ENST00000537196.1. In some embodiments, the ASO targets a ELP1 pre-mRNA sequence comprising a NSAE exon. In some embodiments, the ASO targets a ELP1 pre-mRNA sequence comprising an intron flanking the 3′ splice site of the NSAE exon. In some embodiments, the ASO targets a ELP1 pre-mRNA sequence comprising an intron flanking the 5′ splice site of the NSAE exon. In some embodiments, the transcript is selected from the group consisting of transcript_ENST00000374647.10, transcript_ENST00000467959.1, transcript_ENST00000495759.5, and transcript_ENST00000537196.1.

In some embodiments, the ELP1 pre-mRNA transcript is encoded by a genetic sequence with at least about 80%, 85%, 90%, 95%, 96%, 97%, 98%, 99% or 100% sequence identity to the Ensembl reference number ENSG00000070061.15 or a complement thereof. In some embodiments, the ELP1 pre-mRNA transcript comprises a sequence with at least about 80%, 85%, 90%, 95%, 96%, 97%, 98%, 99% or 100% sequence identity to a ELP1 pre-mRNA transcript or a complement thereof described herein.

In some embodiments, the targeted portion of the ELP1 pre-mRNA comprises a sequence with at least 80%, 85%, 90%, 95%, 97%, or 100% sequence identity to a region comprising at least 8 contiguous nucleic acids of a sequence of premrna_ENST00000374647.10, premrna_ENST00000467959.1, premrna_ENST00000495759.5, premrna_ENST00000537196.1 or a sequence of Table 3 or complements thereof. In some embodiments, the targeted portion of the ELP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 473-SEQ ID NO: 476 or complements thereof. In some embodiments, the targeted portion of the ELP1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ENTPD4 genomic sequence. In some embodiments, the ASO targets a NSAE pre-mRNA transcript from a ENTPD4 genomic sequence comprising a NSAE exon. In some embodiments, the ASO targets a NSAE pre-mRNA transcript from a ENTPD4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000356206.10, premrna_ENST00000358689.9, premrna_ENST00000417069.6, premrna_ENST00000518471.5, premrna_ENST00000518718.1, premrna_ENST00000519839.1, premrna_ENST00000520936.1, premrna_ENST00000521321.5, premrna_ENST00000522255.1, premrna_ENST00000522913.1, and premrna_ENST00000523958.5. In some embodiments, the ASO targets a ENTPD4 pre-mRNA sequence comprising a NSAE exon. In some embodiments, the ASO targets a ENTPD4 pre-mRNA sequence comprising an intron flanking the 3′ splice site of the NSAE exon. In some embodiments, the ASO targets a ENTPD4 pre-mRNA sequence comprising an intron flanking the 5′ splice site of the NSAE exon. In some embodiments, the transcript is selected from the group consisting of transcript_ENST00000356206.10, transcript_ENST00000358689.9, transcript_ENST00000417069.6, transcript_ENST00000518471.5, transcript_ENST00000518718.1, transcript_ENST00000519839.1, transcript_ENST00000520936.1, transcript_ENST00000521321.5, transcript_ENST00000522255.1, transcript_ENST00000522913.1, and transcript_ENST00000523958.5.

In some embodiments, the ENTPD4 pre-mRNA transcript is encoded by a genetic sequence with at least about 80%, 85%, 90%, 95%, 96%, 97%, 98%, 99% or 100% sequence identity to the Ensembl reference number ENSG00000197217.13 or a complement thereof. In some embodiments, the ENTPD4 pre-mRNA transcript comprises a sequence with at least about 80%, 85%, 90%, 95%, 96%, 97%, 98%, 99% or 100% sequence identity to a ENTPD4 pre-mRNA transcript or a complement thereof described herein.

In some embodiments, the targeted portion of the ENTPD4 pre-mRNA comprises a sequence with at least 80%, 85%, 90%, 95%, 97%, or 100% sequence identity to a region comprising at least 8 contiguous nucleic acids of a sequence of premrna_ENST00000356206.10, premrna_ENST00000358689.9, premrna_ENST00000417069.6, premrna_ENST00000518471.5, premrna_ENST00000518718.1, premrna_ENST00000519839.1, premrna_ENST00000520936.1, premrna_ENST00000521321.5, premrna_ENST00000522255.1, premrna_ENST00000522913.1, premrna_ENST00000523958.5 or a sequence of Table 3 or complements thereof. In some embodiments, the targeted portion of the ENTPD4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 477-SEQ ID NO: 480 or complements thereof. In some embodiments, the targeted portion of the ENTPD4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ESRRA genomic sequence. In some embodiments, the ASO targets a NSAE pre-mRNA transcript from a ESRRA genomic sequence comprising a NSAE exon. In some embodiments, the ASO targets a NSAE pre-mRNA transcript from a ESRRA genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000000442.11, premrna_ENST00000405666.5, premrna_ENST00000406310.6, premrna_ENST00000467987.1, premrna_ENST00000468670.1, premrna_ENST00000539594.5, premrna_ENST00000545035.1, and premrna_ENST00000673656.1. In some embodiments, the ASO targets a ESRRA pre-mRNA sequence comprising a NSAE exon. In some embodiments, the ASO targets a ESRRA pre-mRNA sequence comprising an intron flanking the 3′ splice site of the NSAE exon. In some embodiments, the ASO targets a ESRRA pre-mRNA sequence comprising an intron flanking the 5′ splice site of the NSAE exon. In some embodiments, the transcript is selected from the group consisting of transcript_ENST00000000442.11, transcript_ENST00000405666.5, transcript_ENST00000406310.6, transcript_ENST00000467987.1, transcript_ENST00000468670.1, transcript_ENST00000539594.5, transcript_ENST00000545035.1, and transcript_ENST00000673656.1.

In some embodiments, the ESRRA pre-mRNA transcript is encoded by a genetic sequence with at least about 80%, 85%, 90%, 95%, 96%, 97%, 98%, 99% or 100% sequence identity to the Ensembl reference number ENSG00000173153.16 or a complement thereof. In some embodiments, the ESRRA pre-mRNA transcript comprises a sequence with at least about 80%, 85%, 90%, 95%, 96%, 97%, 98%, 99% or 100% sequence identity to a ESRRA pre-mRNA transcript or a complement thereof described herein.

In some embodiments, the targeted portion of the ESRRA pre-mRNA comprises a sequence with at least 80%, 85%, 90%, 95%, 97%, or 100% sequence identity to a region comprising at least 8 contiguous nucleic acids of a sequence of premrna_ENST00000000442.11, premrna_ENST00000405666.5, premrna_ENST00000406310.6, premrna_ENST00000467987.1, premrna_ENST00000468670.1, premrna_ENST00000539594.5, premrna_ENST00000545035.1, premrna_ENST00000673656.1 or a sequence of Table 3 or complements thereof. In some embodiments, the targeted portion of the ESRRA pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 481-SEQ ID NO: 484 or complements thereof. In some embodiments, the targeted portion of the ESRRA pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ESS2 genomic sequence. In some embodiments, the ASO targets a NSAE pre-mRNA transcript from a ESS2 genomic sequence comprising a NSAE exon. In some embodiments, the ASO targets a NSAE pre-mRNA transcript from a ESS2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000252137.11, premrna_ENST00000434568.5, premrna_ENST00000469466.1, and premrna_ENST00000472073.1. In some embodiments, the ASO targets a ESS2 pre-mRNA sequence comprising a NSAE exon. In some embodiments, the ASO targets a ESS2 pre-mRNA sequence comprising an intron flanking the 3′ splice site of the NSAE exon. In some embodiments, the ASO targets a ESS2 pre-mRNA sequence comprising an intron flanking the 5′ splice site of the NSAE exon. In some embodiments, the transcript is selected from the group consisting of transcript_ENST00000252137.11, transcript_ENST00000434568.5, transcript_ENST00000469466.1, and transcript_ENST00000472073.1.

In some embodiments, the ESS2 pre-mRNA transcript is encoded by a genetic sequence with at least about 80%, 85%, 90%, 95%, 96%, 97%, 98%, 99% or 100% sequence identity to the Ensembl reference number ENSG00000100056.12 or a complement thereof. In some embodiments, the ESS2 pre-mRNA transcript comprises a sequence with at least about 80%, 85%, 90%, 95%, 96%, 97%, 98%, 99% or 100% sequence identity to a ESS2 pre-mRNA transcript or a complement thereof described herein.

In some embodiments, the targeted portion of the ESS2 pre-mRNA comprises a sequence with at least 80%, 85%, 90%, 95%, 97%, or 100% sequence identity to a region comprising at least 8 contiguous nucleic acids of a sequence of premrna_ENST00000252137.11, premrna_ENST00000434568.5, premrna_ENST00000469466.1, premrna_ENST00000472073.1 or a sequence of Table 3 or complements thereof. In some embodiments, the targeted portion of the ESS2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 485-SEQ ID NO: 488 or complements thereof. In some embodiments, the targeted portion of the ESS2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ETAA1 genomic sequence. In some embodiments, the ASO targets a NSAE pre-mRNA transcript from a ETAA1 genomic sequence comprising a NSAE exon. In some embodiments, the ASO targets a NSAE pre-mRNA transcript from a ETAA1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000272342.6, premrna_ENST00000462772.2, premrna_ENST00000644028.1, and premrna_ENST00000645739.1. In some embodiments, the ASO targets a ETAA1 pre-mRNA sequence comprising a NSAE exon. In some embodiments, the ASO targets a ETAA1 pre-mRNA sequence comprising an intron flanking the 3′ splice site of the NSAE exon. In some embodiments, the ASO targets a ETAA1 pre-mRNA sequence comprising an intron flanking the 5′ splice site of the NSAE exon. In some embodiments, the transcript is selected from the group consisting of transcript_ENST00000272342.6, transcript_ENST00000462772.2, transcript_ENST00000644028.1, and transcript_ENST00000645739.1.

In some embodiments, the ETAA1 pre-mRNA transcript is encoded by a genetic sequence with at least about 80%, 85%, 90%, 95%, 96%, 97%, 98%, 99% or 100% sequence identity to the Ensembl reference number ENSG00000143971.9 or a complement thereof. In some embodiments, the ETAA1 pre-mRNA transcript comprises a sequence with at least about 80%, 85%, 90%, 95%, 96%, 97%, 98%, 99% or 100% sequence identity to a ETAA1 pre-mRNA transcript or a complement thereof described herein.

In some embodiments, the targeted portion of the ETAA1 pre-mRNA comprises a sequence with at least 80%, 85%, 90%, 95%, 97%, or 100% sequence identity to a region comprising at least 8 contiguous nucleic acids of a sequence of premrna_ENST00000272342.6, premrna_ENST00000462772.2, premrna_ENST00000644028.1, premrna_ENST00000645739.1 or a sequence of Table 3 or complements thereof. In some embodiments, the targeted portion of the ETAA1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 489-SEQ ID NO: 492 or complements thereof. In some embodiments, the targeted portion of the ETAA1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ETHE1 genomic sequence. In some embodiments, the ASO targets a NSAE pre-mRNA transcript from a ETHE1 genomic sequence comprising a NSAE exon. In some embodiments, the ASO targets a NSAE pre-mRNA transcript from a ETHE1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000292147.7, premrna_ENST00000594342.5, premrna_ENST00000595115.1, premrna_ENST00000598330.1, premrna_ENST00000600651.5, and premrna_ENST00000602138.1. In some embodiments, the ASO targets a ETHE1 pre-mRNA sequence comprising a NSAE exon. In some embodiments, the ASO targets a ETHE1 pre-mRNA sequence comprising an intron flanking the 3′ splice site of the NSAE exon. In some embodiments, the ASO targets a ETHE1 pre-mRNA sequence comprising an intron flanking the 5′ splice site of the NSAE exon. In some embodiments, the transcript is selected from the group consisting of transcript_ENST00000292147.7, transcript_ENST00000594342.5, transcript_ENST00000595115.1, transcript_ENST00000598330.1, transcript_ENST00000600651.5, and transcript_ENST00000602138.1.

In some embodiments, the ETHE1 pre-mRNA transcript is encoded by a genetic sequence with at least about 80%, 85%, 90%, 95%, 96%, 97%, 98%, 99% or 100% sequence identity to the Ensembl reference number ENSG00000105755.8 or a complement thereof. In some embodiments, the ETHE1 pre-mRNA transcript comprises a sequence with at least about 80%, 85%, 90%, 95%, 96%, 97%, 98%, 99% or 100% sequence identity to a ETHE1 pre-mRNA transcript or a complement thereof described herein.

In some embodiments, the targeted portion of the ETHE1 pre-mRNA comprises a sequence with at least 80%, 85%, 90%, 95%, 97%, or 100% sequence identity to a region comprising at least 8 contiguous nucleic acids of a sequence of premrna_ENST00000292147.7, premrna_ENST00000594342.5, premrna_ENST00000595115.1, premrna_ENST00000598330.1, premrna_ENST00000600651.5, premrna_ENST00000602138.1 or a sequence of Table 3 or complements thereof. In some embodiments, the targeted portion of the ETHE1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 493-SEQ ID NO: 496 or complements thereof. In some embodiments, the targeted portion of the ETHE1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a FANCA genomic sequence. In some embodiments, the ASO targets a NSAE pre-mRNA transcript from a FANCA genomic sequence comprising a NSAE exon. In some embodiments, the ASO targets a NSAE pre-mRNA transcript from a FANCA genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000305699.15, premrna_ENST00000389301.8, premrna_ENST00000389302.7, premrna_ENST00000534992.5, premrna_ENST00000543736.5, premrna_ENST00000561660.1, premrna_ENST00000561667.1, premrna_ENST00000561722.5, premrna_ENST00000562424.1, premrna_ENST00000563318.1, premrna_ENST00000563510.5, premrna_ENST00000563513.1, premrna_ENST00000563673.5, premrna_ENST00000563767.1, premrna_ENST00000564475.5, premrna_ENST00000564870.1, premrna_ENST00000564969.5, premrna_ENST00000565582.5, premrna_ENST00000566133.1, premrna_ENST00000566409.1, premrna_ENST00000566889.5, premrna_ENST00000567205.1, premrna_ENST00000567284.6, premrna_ENST00000567510.1, premrna_ENST00000567621.5, premrna_ENST00000567879.5, premrna_ENST00000567883.5, premrna_ENST00000567943.1, premrna_ENST00000567988.5, premrna_ENST00000568369.5, premrna_ENST00000568626.1, and premrna_ENST00000568983.5. In some embodiments, the ASO targets a FANCA pre-mRNA sequence comprising a NSAE exon. In some embodiments, the ASO targets a FANCA pre-mRNA sequence comprising an intron flanking the 3′ splice site of the NSAE exon. In some embodiments, the ASO targets a FANCA pre-mRNA sequence comprising an intron flanking the 5′ splice site of the NSAE exon. In some embodiments, the transcript is selected from the group consisting of transcript_ENST00000305699.15, transcript_ENST00000389301.8, transcript_ENST00000389302.7, transcript_ENST00000534992.5, transcript_ENST00000543736.5, transcript_ENST00000561660.1, transcript_ENST00000561667.1, transcript_ENST00000561722.5, transcript_ENST00000562424.1, transcript_ENST00000563318.1, transcript_ENST00000563510.5, transcript_ENST00000563513.1, transcript_ENST00000563673.5, transcript_ENST00000563767.1, transcript_ENST00000564475.5, transcript_ENST00000564870.1, transcript_ENST00000564969.5, transcript_ENST00000565582.5, transcript_ENST00000566133.1, transcript_ENST00000566409.1, transcript_ENST00000566889.5, transcript_ENST00000567205.1, transcript_ENST00000567284.6, transcript_ENST00000567510.1, transcript_ENST00000567621.5, transcript_ENST00000567879.5, transcript_ENST00000567883.5, transcript_ENST00000567943.1, transcript_ENST00000567988.5, transcript_ENST00000568369.5, transcript_ENST00000568626.1, and transcript_ENST00000568983.5.

In some embodiments, the FANCA pre-mRNA transcript is encoded by a genetic sequence with at least about 80%, 85%, 90%, 95%, 96%, 97%, 98%, 99% or 100% sequence identity to the Ensembl reference number ENSG00000187741.15 or a complement thereof. In some embodiments, the FANCA pre-mRNA transcript comprises a sequence with at least about 80%, 85%, 90%, 95%, 96%, 97%, 98%, 99% or 100% sequence identity to a FANCA pre-mRNA transcript or a complement thereof described herein.

In some embodiments, the targeted portion of the FANCA pre-mRNA comprises a sequence with at least 80%, 85%, 90%, 95%, 97%, or 100% sequence identity to a region comprising at least 8 contiguous nucleic acids of a sequence of premrna_ENST00000305699.15, premrna_ENST00000389301.8, premrna_ENST00000389302.7, premrna_ENST00000534992.5, premrna_ENST00000543736.5, premrna_ENST00000561660.1, premrna_ENST00000561667.1, premrna_ENST00000561722.5, premrna_ENST00000562424.1, premrna_ENST00000563318.1, premrna_ENST00000563510.5, premrna_ENST00000563513.1, premrna_ENST00000563673.5, premrna_ENST00000563767.1, premrna_ENST00000564475.5, premrna_ENST00000564870.1, premrna_ENST00000564969.5, premrna_ENST00000565582.5, premrna_ENST00000566133.1, premrna_ENST00000566409.1, premrna_ENST00000566889.5, premrna_ENST00000567205.1, premrna_ENST00000567284.6, premrna_ENST00000567510.1, premrna_ENST00000567621.5, premrna_ENST00000567879.5, premrna_ENST00000567883.5, premrna_ENST00000567943.1, premrna_ENST00000567988.5, premrna_ENST00000568369.5, premrna_ENST00000568626.1, premrna_ENST00000568983.5 or a sequence of Table 3 or complements thereof. In some embodiments, the targeted portion of the FANCA pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 497-SEQ ID NO: 502 or complements thereof. In some embodiments, the targeted portion of the FANCA pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a FASTK genomic sequence. In some embodiments, the ASO targets a NSAE pre-mRNA transcript from a FASTK genomic sequence comprising a NSAE exon. In some embodiments, the ASO targets a NSAE pre-mRNA transcript from a FASTK genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000297532.11, premrna_ENST00000353841.6, premrna_ENST00000459800.1, premrna_ENST00000460980.5, premrna_ENST00000461979.1, premrna_ENST00000465272.1, premrna_ENST00000466855.1, premrna_ENST00000467237.5, premrna_ENST00000469237.5, premrna_ENST00000478477.1, premrna_ENST00000478883.1, premrna_ENST00000482571.2, premrna_ENST00000482806.5, premrna_ENST00000483953.5, premrna_ENST00000489884.5, premrna_ENST00000496663.1, and premrna_ENST00000540185.5. In some embodiments, the ASO targets a FASTK pre-mRNA sequence comprising a NSAE exon. In some embodiments, the ASO targets a FASTK pre-mRNA sequence comprising an intron flanking the 3′ splice site of the NSAE exon. In some embodiments, the ASO targets a FASTK pre-mRNA sequence comprising an intron flanking the 5′ splice site of the NSAE exon. In some embodiments, the transcript is selected from the group consisting of transcript_ENST00000297532.11, transcript_ENST00000353841.6, transcript_ENST00000459800.1, transcript_ENST00000460980.5, transcript_ENST00000461979.1, transcript_ENST00000465272.1, transcript_ENST00000466855.1, transcript_ENST00000467237.5, transcript_ENST00000469237.5, transcript_ENST00000478477.1, transcript_ENST00000478883.1, transcript_ENST00000482571.2, transcript_ENST00000482806.5, transcript_ENST00000483953.5, transcript_ENST00000489884.5, transcript_ENST00000496663.1, and transcript_ENST00000540185.5.

In some embodiments, the FASTK pre-mRNA transcript is encoded by a genetic sequence with at least about 80%, 85%, 90%, 95%, 96%, 97%, 98%, 99% or 100% sequence identity to the Ensembl reference number ENSG00000164896.20 or a complement thereof. In some embodiments, the FASTK pre-mRNA transcript comprises a sequence with at least about 80%, 85%, 90%, 95%, 96%, 97%, 98%, 99% or 100% sequence identity to a FASTK pre-mRNA transcript or a complement thereof described herein.

In some embodiments, the targeted portion of the FASTK pre-mRNA comprises a sequence with at least 80%, 85%, 90%, 95%, 97%, or 100% sequence identity to a region comprising at least 8 contiguous nucleic acids of a sequence of premrna_ENST00000297532.11, premrna_ENST00000353841.6, premrna_ENST00000459800.1, premrna_ENST00000460980.5, premrna_ENST00000461979.1, premrna_ENST00000465272.1, premrna_ENST00000466855.1, premrna_ENST00000467237.5, premrna_ENST00000469237.5, premrna_ENST00000478477.1, premrna_ENST00000478883.1, premrna_ENST00000482571.2, premrna_ENST00000482806.5, premrna_ENST00000483953.5, premrna_ENST00000489884.5, premrna_ENST00000496663.1, premrna_ENST00000540185.5, or a sequence of Table 3 or complements thereof. In some embodiments, the targeted portion of the FASTK pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 503-SEQ ID NO: 517 or complements thereof. In some embodiments, the targeted portion of the FASTK pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a FASTKD3 genomic sequence. In some embodiments, the ASO targets a NSAE pre-mRNA transcript from a FASTKD3 genomic sequence comprising a NSAE exon. In some embodiments, the ASO targets a NSAE pre-mRNA transcript from a FASTKD3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000264669.10, premrna_ENST00000282110.8, premrna_ENST00000504695.1, premrna_ENST00000507036.1, premrna_ENST00000507572.1, premrna_ENST00000511261.5, premrna_ENST00000513577.1, and premrna_ENST00000513658.5. In some embodiments, the ASO targets a FASTKD3 pre-mRNA sequence comprising a NSAE exon. In some embodiments, the ASO targets a FASTKD3 pre-mRNA sequence comprising an intron flanking the 3′ splice site of the NSAE exon. In some embodiments, the ASO targets a FASTKD3 pre-mRNA sequence comprising an intron flanking the 5′ splice site of the NSAE exon. In some embodiments, the transcript is selected from the group consisting of transcript_ENST00000264669.10, transcript_ENST00000282110.8, transcript_ENST00000504695.1, transcript_ENST00000507036.1, transcript_ENST00000507572.1, transcript_ENST00000511261.5, transcript_ENST00000513577.1, and transcript_ENST00000513658.5.

In some embodiments, the FASTKD3 pre-mRNA transcript is encoded by a genetic sequence with at least about 80%, 85%, 90%, 95%, 96%, 97%, 98%, 99% or 100% sequence identity to the Ensembl reference number ENSG00000124279.12 or a complement thereof. In some embodiments, the FASTKD3 pre-mRNA transcript comprises a sequence with at least about 80%, 85%, 90%, 95%, 96%, 97%, 98%, 99% or 100% sequence identity to a FASTKD3 pre-mRNA transcript or a complement thereof described herein.

In some embodiments, the targeted portion of the FASTKD3 pre-mRNA comprises a sequence with at least 80%, 85%, 90%, 95%, 97%, or 100% sequence identity to a region comprising at least 8 contiguous nucleic acids of a sequence of premrna_ENST00000264669.10, premrna_ENST00000282110.8, premrna_ENST00000504695.1, premrna_ENST00000507036.1, premrna_ENST00000507572.1, premrna_ENST00000511261.5, premrna_ENST00000513577.1, premrna_ENST00000513658.5 or a sequence of Table 3 or complements thereof. In some embodiments, the targeted portion of the FASTKD3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 518-SEQ ID NO: 521 or complements thereof. In some embodiments, the targeted portion of the FASTKD3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a FPGS genomic sequence. In some embodiments, the ASO targets a NSAE pre-mRNA transcript from a FPGS genomic sequence comprising a NSAE exon. In some embodiments, the ASO targets a NSAE pre-mRNA transcript from a FPGS genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000373225.7, premrna_ENST00000373228.5, premrna_ENST00000373247.7, premrna_ENST00000393706.6, premrna_ENST00000423577.1, premrna_ENST00000431857.5, premrna_ENST00000460181.5, premrna_ENST00000467826.5, premrna_ENST00000469310.1, premrna_ENST00000473536.1, premrna_ENST00000475270.1, premrna_ENST00000475765.5, premrna_ENST00000479147.6, premrna_ENST00000479375.6, premrna_ENST00000481552.6, premrna_ENST00000488307.5, premrna_ENST00000488506.5, premrna_ENST00000489522.5, premrna_ENST00000496586.5, premrna_ENST00000497386.5, and premrna_ENST00000630236.2. In some embodiments, the ASO targets a FPGS pre-mRNA sequence comprising a NSAE exon. In some embodiments, the ASO targets a FPGS pre-mRNA sequence comprising an intron flanking the 3′ splice site of the NSAE exon. In some embodiments, the ASO targets a FPGS pre-mRNA sequence comprising an intron flanking the 5′ splice site of the NSAE exon. In some embodiments, the transcript is selected from the group consisting of transcript_ENST00000373225.7, transcript_ENST00000373228.5, transcript_ENST00000373247.7, transcript_ENST00000393706.6, transcript_ENST00000423577.1, transcript_ENST00000431857.5, transcript_ENST00000460181.5, transcript_ENST00000467826.5, transcript_ENST00000469310.1, transcript_ENST00000473536.1, transcript_ENST00000475270.1, transcript_ENST00000475765.5, transcript_ENST00000479147.6, transcript_ENST00000479375.6, transcript_ENST00000481552.6, transcript_ENST00000488307.5, transcript_ENST00000488506.5, transcript_ENST00000489522.5, transcript_ENST00000496586.5, transcript_ENST00000497386.5, and transcript_ENST00000630236.2.

In some embodiments, the FPGS pre-mRNA transcript is encoded by a genetic sequence with at least about 80%, 85%, 90%, 95%, 96%, 97%, 98%, 99% or 100% sequence identity to the Ensembl reference number ENSG00000136877.15 or a complement thereof. In some embodiments, the FPGS pre-mRNA transcript comprises a sequence with at least about 80%, 85%, 90%, 95%, 96%, 97%, 98%, 99% or 100% sequence identity to a FPGS pre-mRNA transcript or a complement thereof described herein.

In some embodiments, the targeted portion of the FPGS pre-mRNA comprises a sequence with at least 80%, 85%, 90%, 95%, 97%, or 100% sequence identity to a region comprising at least 8 contiguous nucleic acids of a sequence of premrna_ENST00000373225.7, premrna_ENST00000373228.5, premrna_ENST00000373247.7, premrna_ENST00000393706.6, premrna_ENST00000423577.1, premrna_ENST00000431857.5, premrna_ENST00000460181.5, premrna_ENST00000467826.5, premrna_ENST00000469310.1, premrna_ENST00000473536.1, premrna_ENST00000475270.1, premrna_ENST00000475765.5, premrna_ENST00000479147.6, premrna_ENST00000479375.6, premrna_ENST00000481552.6, premrna_ENST00000488307.5, premrna_ENST00000488506.5, premrna_ENST00000489522.5, premrna_ENST00000496586.5, premrna_ENST00000497386.5, premrna_ENST00000630236.2 or a sequence of Table 3 or complements thereof. In some embodiments, the targeted portion of the FPGS pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 522-SEQ ID NO: 525 or complements thereof. In some embodiments, the targeted portion of the FPGS pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a FREM1 genomic sequence. In some embodiments, the ASO targets a NSAE pre-mRNA transcript from a FREM1 genomic sequence comprising a NSAE exon. In some embodiments, the ASO targets a NSAE pre-mRNA transcript from a FREM1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000380875.7, premrna_ENST00000380880.3, premrna_ENST00000380894.5, premrna_ENST00000422223.6, premrna_ENST00000427623.5, premrna_ENST00000466679.1, premrna_ENST00000485068.5, premrna_ENST00000486223.1, and premrna_ENST00000497634.2. In some embodiments, the ASO targets a FREM1 pre-mRNA sequence comprising a NSAE exon. In some embodiments, the ASO targets a FREM1 pre-mRNA sequence comprising an intron flanking the 3′ splice site of the NSAE exon. In some embodiments, the ASO targets a FREM1 pre-mRNA sequence comprising an intron flanking the 5′ splice site of the NSAE exon. In some embodiments, the transcript is selected from the group consisting of transcript_ENST00000380875.7, transcript_ENST00000380880.3, transcript_ENST00000380894.5, transcript_ENST00000422223.6, transcript_ENST00000427623.5, transcript_ENST00000466679.1, transcript_ENST00000485068.5, transcript_ENST00000486223.1, and transcript_ENST00000497634.2.

In some embodiments, the FREM1 pre-mRNA transcript is encoded by a genetic sequence with at least about 80%, 85%, 90%, 95%, 96%, 97%, 98%, 99% or 100% sequence identity to the Ensembl reference number ENSG00000164946.19 or a complement thereof. In some embodiments, the FREM1 pre-mRNA transcript comprises a sequence with at least about 80%, 85%, 90%, 95%, 96%, 97%, 98%, 99% or 100% sequence identity to a FREM1 pre-mRNA transcript or a complement thereof described herein.

In some embodiments, the targeted portion of the FREM1 pre-mRNA comprises a sequence with at least 80%, 85%, 90%, 95%, 97%, or 100% sequence identity to a region comprising at least 8 contiguous nucleic acids of a sequence of premrna_ENST00000380875.7, premrna_ENST00000380880.3, premrna_ENST00000380894.5, premrna_ENST00000422223.6, premrna_ENST00000427623.5, premrna_ENST00000466679.1, premrna_ENST00000485068.5, premrna_ENST00000486223.1, premrna_ENST00000497634.2, or a sequence of Table 3 or complements thereof. In some embodiments, the targeted portion of the FREM1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 526-SEQ ID NO: 531 or complements thereof. In some embodiments, the targeted portion of the FREM1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GAS8 genomic sequence. In some embodiments, the ASO targets a NSAE pre-mRNA transcript from a GAS8 genomic sequence comprising a NSAE exon. In some embodiments, the ASO targets a NSAE pre-mRNA transcript from a GAS8 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000268699.9, premrna_ENST00000536122.7, premrna_ENST00000537797.5, premrna_ENST00000540721.5, premrna_ENST00000561675.1, premrna_ENST00000563980.5, premrna_ENST00000564392.5, premrna_ENST00000564789.5, premrna_ENST00000564802.1, premrna_ENST00000564853.1, premrna_ENST00000565000.1, premrna_ENST00000565062.5, premrna_ENST00000565957.5, premrna_ENST00000566266.5, premrna_ENST00000568284.1, premrna_ENST00000568664.5, premrna_ENST00000568705.5, premrna_ENST00000569399.1, premrna_ENST00000569558.5, and premrna_ENST00000620723.4. In some embodiments, the ASO targets a GAS8 pre-mRNA sequence comprising a NSAE exon. In some embodiments, the ASO targets a GAS8 pre-mRNA sequence comprising an intron flanking the 3′ splice site of the NSAE exon. In some embodiments, the ASO targets a GAS8 pre-mRNA sequence comprising an intron flanking the 5′ splice site of the NSAE exon. In some embodiments, the transcript is selected from the group consisting of transcript_ENST00000268699.9, transcript_ENST00000536122.7, transcript_ENST00000537797.5, transcript_ENST00000540721.5, transcript_ENST00000561675.1, transcript_ENST00000563980.5, transcript_ENST00000564392.5, transcript_ENST00000564789.5, transcript_ENST00000564802.1, transcript_ENST00000564853.1, transcript_ENST00000565000.1, transcript_ENST00000565062.5, transcript_ENST00000565957.5, transcript_ENST00000566266.5, transcript_ENST00000568284.1, transcript_ENST00000568664.5, transcript_ENST00000568705.5, transcript_ENST00000569399.1, transcript_ENST00000569558.5, and transcript_ENST00000620723.4.

In some embodiments, the GAS8 pre-mRNA transcript is encoded by a genetic sequence with at least about 80%, 85%, 90%, 95%, 96%, 97%, 98%, 99% or 100% sequence identity to the Ensembl reference number ENSG00000141013.17 or a complement thereof. In some embodiments, the GAS8 pre-mRNA transcript comprises a sequence with at least about 80%, 85%, 90%, 95%, 96%, 97%, 98%, 99% or 100% sequence identity to a GAS8 pre-mRNA transcript or a complement thereof described herein.

In some embodiments, the targeted portion of the GAS8 pre-mRNA comprises a sequence with at least 80%, 85%, 90%, 95%, 97%, or 100% sequence identity to a region comprising at least 8 contiguous nucleic acids of a sequence of premrna_ENST00000268699.9, premrna_ENST00000536122.7, premrna_ENST00000537797.5, premrna_ENST00000540721.5, premrna_ENST00000561675.1, premrna_ENST00000563980.5, premrna_ENST00000564392.5, premrna_ENST00000564789.5, premrna_ENST00000564802.1, premrna_ENST00000564853.1, premrna_ENST00000565000.1, premrna_ENST00000565062.5, premrna_ENST00000565957.5, premrna_ENST00000566266.5, premrna_ENST00000568284.1, premrna_ENST00000568664.5, premrna_ENST00000568705.5, premrna_ENST00000569399.1, premrna_ENST00000569558.5, premrna_ENST00000620723.4 or a sequence of Table 3 or complements thereof. In some embodiments, the targeted portion of the GAS8 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 532-SEQ ID NO: 539 or complements thereof. In some embodiments, the targeted portion of the GAS8 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GCK genomic sequence. In some embodiments, the ASO targets a NSAE pre-mRNA transcript from a GCK genomic sequence comprising a NSAE exon. In some embodiments, the ASO targets a NSAE pre-mRNA transcript from a GCK genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000336642.9, premrna_ENST00000345378.7, premrna_ENST00000395796.7, premrna_ENST00000403799.8, premrna_ENST00000437084.1, premrna_ENST00000459642.1, premrna_ENST00000473353.1, premrna_ENST00000476008.1, premrna_ENST00000616242.4, premrna_ENST00000671824.1, premrna_ENST00000672743.1, and premrna_ENST00000673284.1. In some embodiments, the ASO targets a GCK pre-mRNA sequence comprising a NSAE exon. In some embodiments, the ASO targets a GCK pre-mRNA sequence comprising an intron flanking the 3′ splice site of the NSAE exon. In some embodiments, the ASO targets a GCK pre-mRNA sequence comprising an intron flanking the 5′ splice site of the NSAE exon. In some embodiments, the transcript is selected from the group consisting of transcript_ENST00000336642.9, transcript_ENST00000345378.7, transcript_ENST00000395796.7, transcript_ENST00000403799.8, transcript_ENST00000437084.1, transcript_ENST00000459642.1, transcript_ENST00000473353.1, transcript_ENST00000476008.1, transcript_ENST00000616242.4, transcript_ENST00000671824.1, transcript_ENST00000672743.1, and transcript_ENST00000673284.1.

In some embodiments, the GCK pre-mRNA transcript is encoded by a genetic sequence with at least about 80%, 85%, 90%, 95%, 96%, 97%, 98%, 99% or 100% sequence identity to the Ensembl reference number ENSG00000106633.17 or a complement thereof. In some embodiments, the GCK pre-mRNA transcript comprises a sequence with at least about 80%, 85%, 90%, 95%, 96%, 97%, 98%, 99% or 100% sequence identity to a GCK pre-mRNA transcript or a complement thereof described herein.

In some embodiments, the targeted portion of the GCK pre-mRNA comprises a sequence with at least 80%, 85%, 90%, 95%, 97%, or 100% sequence identity to a region comprising at least 8 contiguous nucleic acids of a sequence of premrna_ENST00000336642.9, premrna_ENST00000345378.7, premrna_ENST00000395796.7, premrna_ENST00000403799.8, premrna_ENST00000437084.1, premrna_ENST00000459642.1, premrna_ENST00000473353.1, premrna_ENST00000476008.1, premrna_ENST00000616242.4, premrna_ENST00000671824.1, premrna_ENST00000672743.1, premrna_ENST00000673284.1 or a sequence of Table 3 or complements thereof. In some embodiments, the targeted portion of the GCK pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 540-SEQ ID NO: 543 or complements thereof. In some embodiments, the targeted portion of the GCK pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GGA1 genomic sequence. In some embodiments, the ASO targets a NSAE pre-mRNA transcript from a GGA1 genomic sequence comprising a NSAE exon. In some embodiments, the ASO targets a NSAE pre-mRNA transcript from a GGA1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000325180.12, premrna_ENST00000326597.6, premrna_ENST00000343632.9, premrna_ENST00000381756.9, premrna_ENST00000405147.7, premrna_ENST00000406772.5, premrna_ENST00000411501.5, premrna_ENST00000413251.5, premrna_ENST00000423024.1, premrna_ENST00000429218.5, premrna_ENST00000431745.5, premrna_ENST00000439161.5, premrna_ENST00000447515.5, premrna_ENST00000449944.5, premrna_ENST00000453208.5, premrna_ENST00000460957.1, premrna_ENST00000463672.1, premrna_ENST00000475445.1, premrna_ENST00000481613.1, premrna_ENST00000484804.5, premrna_ENST00000488672.1, premrna_ENST00000489772.5, and premrna_ENST00000491295.1. In some embodiments, the ASO targets a GGA1 pre-mRNA sequence comprising a NSAE exon. In some embodiments, the ASO targets a GGA1 pre-mRNA sequence comprising an intron flanking the 3′ splice site of the NSAE exon. In some embodiments, the ASO targets a GGA1 pre-mRNA sequence comprising an intron flanking the 5′ splice site of the NSAE exon. In some embodiments, the transcript is selected from the group consisting of transcript_ENST00000325180.12, transcript_ENST00000326597.6, transcript_ENST00000343632.9, transcript_ENST00000381756.9, transcript_ENST00000405147.7, transcript_ENST00000406772.5, transcript_ENST00000411501.5, transcript_ENST00000413251.5, transcript_ENST00000423024.1, transcript_ENST00000429218.5, transcript_ENST00000431745.5, transcript_ENST00000439161.5, transcript_ENST00000447515.5, transcript_ENST00000449944.5, transcript_ENST00000453208.5, transcript_ENST00000460957.1, transcript_ENST00000463672.1, transcript_ENST00000475445.1, transcript_ENST00000481613.1, transcript_ENST00000484804.5, transcript_ENST00000488672.1, transcript_ENST00000489772.5, and transcript_ENST00000491295.1.

In some embodiments, the GGA1 pre-mRNA transcript is encoded by a genetic sequence with at least about 80%, 85%, 90%, 95%, 96%, 97%, 98%, 99% or 100% sequence identity to the Ensembl reference number ENSG00000100083.19 or a complement thereof. In some embodiments, the GGA1 pre-mRNA transcript comprises a sequence with at least about 80%, 85%, 90%, 95%, 96%, 97%, 98%, 99% or 100% sequence identity to a GGA1 pre-mRNA transcript or a complement thereof described herein.

In some embodiments, the targeted portion of the GGA1 pre-mRNA comprises a sequence with at least 80%, 85%, 90%, 95%, 97%, or 100% sequence identity to a region comprising at least 8 contiguous nucleic acids of a sequence of premrna_ENST00000325180.12, premrna_ENST00000326597.6, premrna_ENST00000343632.9, premrna_ENST00000381756.9, premrna_ENST00000405147.7, premrna_ENST00000406772.5, premrna_ENST00000411501.5, premrna_ENST00000413251.5, premrna_ENST00000423024.1, premrna_ENST00000429218.5, premrna_ENST00000431745.5, premrna_ENST00000439161.5, premrna_ENST00000447515.5, premrna_ENST00000449944.5, premrna_ENST00000453208.5, premrna_ENST00000460957.1, premrna_ENST00000463672.1, premrna_ENST00000475445.1, premrna_ENST00000481613.1, premrna_ENST00000484804.5, premrna_ENST00000488672.1, premrna_ENST00000489772.5, premrna_ENST00000491295.1, or a sequence of Table 3 or complements thereof. In some embodiments, the targeted portion of the GGA1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 544-SEQ ID NO: 553 or complements thereof. In some embodiments, the targeted portion of the GGA1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GGA3genomic sequence. In some embodiments, the ASO targets a NSAE pre-mRNA transcript from a GGA3genomic sequence comprising a NSAE exon. In some embodiments, the ASO targets a NSAE pre-mRNA transcript from a GGA3genomic sequence comprising an intron flanking the 3′ splice site of the NSAE exon and an intron flanking the 5′ splice site of a NSAE exon. In some embodiments, the ASO targets a GGA3pre-mRNA sequence comprising a NSAE exon. In some embodiments, the ASO targets a GGA3pre-mRNA sequence comprising an intron flanking the 3′ splice site of the NSAE exon. In some embodiments, the ASO targets a GGA3pre-mRNA sequence comprising an intron flanking the 5′ splice site of the NSAE exon. In some embodiments, the transcript is selected from the group consisting of transcript_ENST00000537686.5, transcript_ENST00000538886.5, transcript_ENST00000577435.5, transcript_ENST00000578208.1, transcript_ENST00000578348.5, transcript_ENST00000578773.1, transcript_ENST00000578896.1, transcript_ENST00000579743.2, transcript_ENST00000580646.5, transcript_ENST00000580799.2, transcript_ENST00000582232.1, transcript_ENST00000582376.1, transcript_ENST00000582486.5, transcript_ENST00000582717.5, transcript_ENST00000582821.5, transcript_ENST00000583282.5, transcript_ENST00000583667.1, transcript_ENST00000584243.5, transcript_ENST00000584550.1, transcript_ENST00000613421.4, transcript_ENST00000614198.4, transcript_ENST00000621217.4, transcript_ENST00000621870.4, and transcript_ENST00000649398.1.

In some embodiments, the GGA3pre-mRNA transcript is encoded by a genetic sequence with at least about 80%, 85%, 90%, 95%, 96%, 97%, 98%, 99% or 100% sequence identity to the Ensembl reference number ENSG00000125447.18 or a complement thereof. In some embodiments, the GGA3pre-mRNA transcript comprises a sequence with at least about 80%, 85%, 90%, 95%, 96%, 97%, 98%, 99% or 100% sequence identity to a GGA3pre-mRNA transcript or a complement thereof described herein.

In some embodiments, the targeted portion of the GGA3pre-mRNA comprises a sequence with at least 80%, 85%, 90%, 95%, 97%, or 100% sequence identity to a region comprising at least 8 contiguous nucleic acids of a sequence of premrna_ENST00000537686.5, premrna_ENST00000538886.5, premrna_ENST00000577435.5, premrna_ENST00000578208.1, premrna_ENST00000578348.5, premrna_ENST00000578773.1, premrna_ENST00000578896.1, premrna_ENST00000579743.2, premrna_ENST00000580646.5, premrna_ENST00000580799.2, premrna_ENST00000582232.1, premrna_ENST00000582376.1, premrna_ENST00000582486.5, premrna_ENST00000582717.5, premrna_ENST00000582821.5, premrna_ENST00000583282.5, premrna_ENST00000583667.1, premrna_ENST00000584243.5, premrna_ENST00000584550.1, premrna_ENST00000613421.4, premrna_ENST00000614198.4, premrna_ENST00000621217.4, premrna_ENST00000621870.4, premrna_ENST00000649398.1 or a sequence of Table 3 or complements thereof. In some embodiments, the targeted portion of the GGA3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 554-SEQ ID NO: 557 or complements thereof. In some embodiments, the targeted portion of the GGA3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GGT1 genomic sequence. In some embodiments, the ASO targets a NSAE pre-mRNA transcript from a GGT1 genomic sequence comprising a NSAE exon. In some embodiments, the ASO targets a NSAE pre-mRNA transcript from a GGT1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000248923.9, premrna_ENST00000400380.5, premrna_ENST00000400382.6, premrna_ENST00000401885.5, premrna_ENST00000403838.5, premrna_ENST00000404223.5, premrna_ENST00000404532.5, premrna_ENST00000404603.5, premrna_ENST00000404920.1, premrna_ENST00000411974.5, premrna_ENST00000412658.5, premrna_ENST00000412898.5, premrna_ENST00000419133.5, premrna_ENST00000425895.5, premrna_ENST00000428855.5, premrna_ENST00000430289.5, premrna_ENST00000432867.5, premrna_ENST00000438643.6, premrna_ENST00000439775.1, premrna_ENST00000445029.5, premrna_ENST00000447416.5, premrna_ENST00000451366.5, premrna_ENST00000452551.5, premrna_ENST00000455483.5, premrna_ENST00000456869.5, premrna_ENST00000466310.5, premrna_ENST00000474191.6, premrna_ENST00000487766.1, premrna_ENST00000490087.1, premrna_ENST00000651180.1, and premrna_ENST00000652248.1. In some embodiments, the ASO targets a GGT1 pre-mRNA sequence comprising a NSAE exon. In some embodiments, the ASO targets a GGT1 pre-mRNA sequence comprising an intron flanking the 3′ splice site of the NSAE exon. In some embodiments, the ASO targets a GGT1 pre-mRNA sequence comprising an intron flanking the 5′ splice site of the NSAE exon. In some embodiments, the transcript is selected from the group consisting of transcript_ENST00000248923.9, transcript_ENST00000400380.5, transcript_ENST00000400382.6, transcript_ENST00000401885.5, transcript_ENST00000403838.5, transcript_ENST00000404223.5, transcript_ENST00000404532.5, transcript_ENST00000404603.5, transcript_ENST00000404920.1, transcript_ENST00000411974.5, transcript_ENST00000412658.5, transcript_ENST00000412898.5, transcript_ENST00000419133.5, transcript_ENST00000425895.5, transcript_ENST00000428855.5, transcript_ENST00000430289.5, transcript_ENST00000432867.5, transcript_ENST00000438643.6, transcript_ENST00000439775.1, transcript_ENST00000445029.5, transcript_ENST00000447416.5, transcript_ENST00000451366.5, transcript_ENST00000452551.5, transcript_ENST00000455483.5, transcript_ENST00000456869.5, transcript_ENST00000466310.5, transcript_ENST00000474191.6, transcript_ENST00000487766.1, transcript_ENST00000490087.1, transcript_ENST00000651180.1, and transcript_ENST00000652248.1.

In some embodiments, the GGT1 pre-mRNA transcript is encoded by a genetic sequence with at least about 80%, 85%, 90%, 95%, 96%, 97%, 98%, 99% or 100% sequence identity to the Ensembl reference number ENSG00000100031.19 or ENSG00000286070.1 or a complement thereof. In some embodiments, the GGT1 pre-mRNA transcript comprises a sequence with at least about 80%, 85%, 90%, 95%, 96%, 97%, 98%, 99% or 100% sequence identity to a GGT1 pre-mRNA transcript or a complement thereof described herein.

In some embodiments, the targeted portion of the GGT1 pre-mRNA comprises a sequence with at least 80%, 85%, 90%, 95%, 97%, or 100% sequence identity to a region comprising at least 8 contiguous nucleic acids of a sequence of premrna_ENST00000248923.9, premrna_ENST00000400380.5, premrna_ENST00000400382.6, premrna_ENST00000401885.5, premrna_ENST00000403838.5, premrna_ENST00000404223.5, premrna_ENST00000404532.5, premrna_ENST00000404603.5, premrna_ENST00000404920.1, premrna_ENST00000411974.5, premrna_ENST00000412658.5, premrna_ENST00000412898.5, premrna_ENST00000419133.5, premrna_ENST00000425895.5, premrna_ENST00000428855.5, premrna_ENST00000430289.5, premrna_ENST00000432867.5, premrna_ENST00000438643.6, premrna_ENST00000439775.1, premrna_ENST00000445029.5, premrna_ENST00000447416.5, premrna_ENST00000451366.5, premrna_ENST00000452551.5, premrna_ENST00000455483.5, premrna_ENST00000456869.5, premrna_ENST00000466310.5, premrna_ENST00000474191.6, premrna_ENST00000487766.1, premrna_ENST00000490087.1, premrna_ENST00000651180.1, premrna_ENST00000652248.1 or a sequence of Table 3 or complements thereof. In some embodiments, the targeted portion of the GGT1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 558-SEQ ID NO: 561 or complements thereof. In some embodiments, the targeted portion of the GGT1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GLMN genomic sequence. In some embodiments, the ASO targets a NSAE pre-mRNA transcript from a GLMN genomic sequence comprising a NSAE exon. In some embodiments, the ASO targets a NSAE pre-mRNA transcript from a GLMN genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000370360.8, premrna_ENST00000463560.1, premrna_ENST00000471465.1, premrna_ENST00000487911.1, premrna_ENST00000495106.5, and premrna_ENST00000495852.6. In some embodiments, the ASO targets a GLMN pre-mRNA sequence comprising a NSAE exon. In some embodiments, the ASO targets a GLMN pre-mRNA sequence comprising an intron flanking the 3′ splice site of the NSAE exon. In some embodiments, the ASO targets a GLMN pre-mRNA sequence comprising an intron flanking the 5′ splice site of the NSAE exon. In some embodiments, the transcript is selected from the group consisting of transcript_ENST00000370360.8, transcript_ENST00000463560.1, transcript_ENST00000471465.1, transcript_ENST00000487911.1, transcript_ENST00000495106.5, and transcript_ENST00000495852.6.

In some embodiments, the GLMN pre-mRNA transcript is encoded by a genetic sequence with at least about 80%, 85%, 90%, 95%, 96%, 97%, 98%, 99% or 100% sequence identity to the Ensembl reference number ENSG00000174842.17 or a complement thereof. In some embodiments, the GLMN pre-mRNA transcript comprises a sequence with at least about 80%, 85%, 90%, 95%, 96%, 97%, 98%, 99% or 100% sequence identity to a GLMN pre-mRNA transcript or a complement thereof described herein.

In some embodiments, the targeted portion of the GLMN pre-mRNA comprises a sequence with at least 80%, 85%, 90%, 95%, 97%, or 100% sequence identity to a region comprising at least 8 contiguous nucleic acids of a sequence of premrna_ENST00000370360.8, premrna_ENST00000463560.1, premrna_ENST00000471465.1, premrna_ENST00000487911.1, premrna_ENST00000495106.5, premrna_ENST00000495852.6 or a sequence of Table 3 or complements thereof. In some embodiments, the targeted portion of the GLMN pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 562-SEQ ID NO: 569 or complements thereof. In some embodiments, the targeted portion of the GLMN pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GON4L genomic sequence. In some embodiments, the ASO targets a NSAE pre-mRNA transcript from a GON4L genomic sequence comprising a NSAE exon. In some embodiments, the ASO targets a NSAE pre-mRNA transcript from a GON4L genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000271883.9, premrna_ENST00000361040.9, premrna_ENST00000368331.5, premrna_ENST00000437809.5, premrna_ENST00000460075.1, premrna_ENST00000466224.1, premrna_ENST00000467009.1, premrna_ENST00000468867.1, premrna_ENST00000471341.5, premrna_ENST00000473267.1, premrna_ENST00000482386.5, premrna_ENST00000483032.1, premrna_ENST00000488251.1, premrna_ENST00000490801.1, premrna_ENST00000496021.1, premrna_ENST00000497369.5, premrna_ENST00000615926.4, premrna_ENST00000620426.4, and premrna_ENST00000622608.1. In some embodiments, the ASO targets a GON4L pre-mRNA sequence comprising a NSAE exon. In some embodiments, the ASO targets a GON4L pre-mRNA sequence comprising an intron flanking the 3′ splice site of the NSAE exon. In some embodiments, the ASO targets a GON4L pre-mRNA sequence comprising an intron flanking the 5′ splice site of the NSAE exon. In some embodiments, the transcript is selected from the group consisting of transcript_ENST00000271883.9, transcript_ENST00000361040.9, transcript_ENST00000368331.5, transcript_ENST00000437809.5, transcript_ENST00000460075.1, transcript_ENST00000466224.1, transcript_ENST00000467009.1, transcript_ENST00000468867.1, transcript_ENST00000471341.5, transcript_ENST00000473267.1, transcript_ENST00000482386.5, transcript_ENST00000483032.1, transcript_ENST00000488251.1, transcript_ENST00000490801.1, transcript_ENST00000496021.1, transcript_ENST00000497369.5, transcript_ENST00000615926.4, transcript_ENST00000620426.4, and transcript_ENST00000622608.1.

In some embodiments, the GON4L pre-mRNA transcript is encoded by a genetic sequence with at least about 80%, 85%, 90%, 95%, 96%, 97%, 98%, 99% or 100% sequence identity to the Ensembl reference number ENSG00000116580.18 or a complement thereof. In some embodiments, the GON4L pre-mRNA transcript comprises a sequence with at least about 80%, 85%, 90%, 95%, 96%, 97%, 98%, 99% or 100% sequence identity to a GON4L pre-mRNA transcript or a complement thereof described herein.

In some embodiments, the targeted portion of the GON4L pre-mRNA comprises a sequence with at least 80%, 85%, 90%, 95%, 97%, or 100% sequence identity to a region comprising at least 8 contiguous nucleic acids of a sequence of premrna_ENST00000271883.9, premrna_ENST00000361040.9, premrna_ENST00000368331.5, premrna_ENST00000437809.5, premrna_ENST00000460075.1, premrna_ENST00000466224.1, premrna_ENST00000467009.1, premrna_ENST00000468867.1, premrna_ENST00000471341.5, premrna_ENST00000473267.1, premrna_ENST00000482386.5, premrna_ENST00000483032.1, premrna_ENST00000488251.1, premrna_ENST00000490801.1, premrna_ENST00000496021.1, premrna_ENST00000497369.5, premrna_ENST00000615926.4, premrna_ENST00000620426.4, premrna_ENST00000622608.1 or a sequence of Table 3 or complements thereof. In some embodiments, the targeted portion of the GON4L pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 570-SEQ ID NO: 573 or complements thereof. In some embodiments, the targeted portion of the GON4L pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a GRB14 genomic sequence. In some embodiments, the ASO targets a NSAE pre-mRNA transcript from a GRB14 genomic sequence comprising a NSAE exon. In some embodiments, the ASO targets a NSAE pre-mRNA transcript from a GRB14 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000263915.8, premrna_ENST00000424693.1, premrna_ENST00000446413.6, premrna_ENST00000469573.1, premrna_ENST00000488342.5, and premrna_ENST00000497306.1. In some embodiments, the ASO targets a GRB14 pre-mRNA sequence comprising a NSAE exon. In some embodiments, the ASO targets a GRB14 pre-mRNA sequence comprising an intron flanking the 3′ splice site of the NSAE exon. In some embodiments, the ASO targets a GRB14 pre-mRNA sequence comprising an intron flanking the 5′ splice site of the NSAE exon. In some embodiments, the transcript is selected from the group consisting of transcript_ENST00000263915.8, transcript_ENST00000424693.1, transcript_ENST00000446413.6, transcript_ENST00000469573.1, transcript_ENST00000488342.5, and transcript_ENST00000497306.1.

In some embodiments, the GRB14 pre-mRNA transcript is encoded by a genetic sequence with at least about 80%, 85%, 90%, 95%, 96%, 97%, 98%, 99% or 100% sequence identity to the Ensembl reference number ENSG00000115290.10 or a complement thereof. In some embodiments, the GRB14 pre-mRNA transcript comprises a sequence with at least about 80%, 85%, 90%, 95%, 96%, 97%, 98%, 99% or 100% sequence identity to a GRB14 pre-mRNA transcript or a complement thereof described herein.

In some embodiments, the targeted portion of the GRB14 pre-mRNA comprises a sequence with at least 80%, 85%, 90%, 95%, 97%, or 100% sequence identity to a region comprising at least 8 contiguous nucleic acids of a sequence of premrna_ENST00000263915.8, premrna_ENST00000424693.1, premrna_ENST00000446413.6, premrna_ENST00000469573.1, premrna_ENST00000488342.5, premrna_ENST00000497306.1 or a sequence of Table 3 or complements thereof. In some embodiments, the targeted portion of the GRB14 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 574-SEQ ID NO: 577 or complements thereof. In some embodiments, the targeted portion of the GRB14 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a HDAC3 genomic sequence. In some embodiments, the ASO targets a NSAE pre-mRNA transcript from a HDAC3 genomic sequence comprising a NSAE exon. In some embodiments, the ASO targets a NSAE pre-mRNA transcript from a HDAC3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000305264.8, premrna_ENST00000459727.5, premrna_ENST00000467533.5, premrna_ENST00000469207.5, premrna_ENST00000469550.6, premrna_ENST00000471968.1, premrna_ENST00000475549.1, premrna_ENST00000476739.5, premrna_ENST00000486618.1, premrna_ENST00000490808.1, premrna_ENST00000491581.5, premrna_ENST00000492407.1, premrna_ENST00000492506.1, premrna_ENST00000495485.1, premrna_ENST00000519474.5, and premrna_ENST00000523088.5. In some embodiments, the ASO targets a HDAC3 pre-mRNA sequence comprising a NSAE exon. In some embodiments, the ASO targets a HDAC3 pre-mRNA sequence comprising an intron flanking the 3′ splice site of the NSAE exon. In some embodiments, the ASO targets a HDAC3 pre-mRNA sequence comprising an intron flanking the 5′ splice site of the NSAE exon. In some embodiments, the transcript is selected from the group consisting of transcript_ENST00000305264.8, transcript_ENST00000459727.5, transcript_ENST00000467533.5, transcript_ENST00000469207.5, transcript_ENST00000469550.6, transcript_ENST00000471968.1, transcript_ENST00000475549.1, transcript_ENST00000476739.5, transcript_ENST00000486618.1, transcript_ENST00000490808.1, transcript_ENST00000491581.5, transcript_ENST00000492407.1, transcript_ENST00000492506.1, transcript_ENST00000495485.1, transcript_ENST00000519474.5, and transcript_ENST00000523088.5.

In some embodiments, the HDAC3 pre-mRNA transcript is encoded by a genetic sequence with at least about 80%, 85%, 90%, 95%, 96%, 97%, 98%, 99% or 100% sequence identity to the Ensembl reference number ENSG00000171720.10 or a complement thereof. In some embodiments, the HDAC3 pre-mRNA transcript comprises a sequence with at least about 80%, 85%, 90%, 95%, 96%, 97%, 98%, 99% or 100% sequence identity to a HDAC3 pre-mRNA transcript or a complement thereof described herein.

In some embodiments, the targeted portion of the HDAC3 pre-mRNA comprises a sequence with at least 80%, 85%, 90%, 95%, 97%, or 100% sequence identity to a region comprising at least 8 contiguous nucleic acids of a sequence of premrna_ENST00000305264.8, premrna_ENST00000459727.5, premrna_ENST00000467533.5, premrna_ENST00000469207.5, premrna_ENST00000469550.6, premrna_ENST00000471968.1, premrna_ENST00000475549.1, premrna_ENST00000476739.5, premrna_ENST00000486618.1, premrna_ENST00000490808.1, premrna_ENST00000491581.5, premrna_ENST00000492407.1, premrna_ENST00000492506.1, premrna_ENST00000495485.1, premrna_ENST00000519474.5, premrna_ENST00000523088.5 or a sequence of Table 3 or complements thereof. In some embodiments, the targeted portion of the HDAC3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 578-SEQ ID NO: 581 or complements thereof. In some embodiments, the targeted portion of the HDAC3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a HELQ genomic sequence. In some embodiments, the ASO targets a NSAE pre-mRNA transcript from a HELQ genomic sequence comprising a NSAE exon. In some embodiments, the ASO targets a NSAE pre-mRNA transcript from a HELQ genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000295488.8, premrna_ENST00000440639.2, premrna_ENST00000508591.5, premrna_ENST00000510985.1, premrna_ENST00000512539.1, and premrna_ENST00000515482.1. In some embodiments, the ASO targets a HELQ pre-mRNA sequence comprising a NSAE exon. In some embodiments, the ASO targets a HELQ pre-mRNA sequence comprising an intron flanking the 3′ splice site of the NSAE exon. In some embodiments, the ASO targets a HELQ pre-mRNA sequence comprising an intron flanking the 5′ splice site of the NSAE exon. In some embodiments, the transcript is selected from the group consisting of transcript_ENST00000295488.8, transcript_ENST00000440639.2, transcript_ENST00000508591.5, transcript_ENST00000510985.1, transcript_ENST00000512539.1, and transcript_ENST00000515482.1.

In some embodiments, the HELQ pre-mRNA transcript is encoded by a genetic sequence with at least about 80%, 85%, 90%, 95%, 96%, 97%, 98%, 99% or 100% sequence identity to the Ensembl reference number ENSG00000163312.11 or a complement thereof. In some embodiments, the HELQ pre-mRNA transcript comprises a sequence with at least about 80%, 85%, 90%, 95%, 96%, 97%, 98%, 99% or 100% sequence identity to a HELQ pre-mRNA transcript or a complement thereof described herein.

In some embodiments, the targeted portion of the HELQ pre-mRNA comprises a sequence with at least 80%, 85%, 90%, 95%, 97%, or 100% sequence identity to a region comprising at least 8 contiguous nucleic acids of a sequence of premrna_ENST00000295488.8, premrna_ENST00000440639.2, premrna_ENST00000508591.5, premrna_ENST00000510985.1, premrna_ENST00000512539.1, premrna_ENST00000515482.1, or a sequence of Table 3 or complements thereof. In some embodiments, the targeted portion of the HELQ pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 582-SEQ ID NO: 585 or complements thereof. In some embodiments, the targeted portion of the HELQ pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a HP1BP3 genomic sequence. In some embodiments, the ASO targets a NSAE pre-mRNA transcript from a HP1BP3 genomic sequence comprising a NSAE exon. In some embodiments, the ASO targets a NSAE pre-mRNA transcript from a HP1BP3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000312239.10, premrna_ENST00000375000.5, premrna_ENST00000375003.6, premrna_ENST00000414993.1, premrna_ENST00000417710.5, premrna_ENST00000419490.5, premrna_ENST00000419948.5, premrna_ENST00000424732.5, premrna_ENST00000437575.5, premrna_ENST00000438032.6, premrna_ENST00000443615.1, premrna_ENST00000487117.1, premrna_ENST00000488722.5, and premrna_ENST00000491748.1. In some embodiments, the ASO targets a HP1BP3 pre-mRNA sequence comprising a NSAE exon. In some embodiments, the ASO targets a HP1BP3 pre-mRNA sequence comprising an intron flanking the 3′ splice site of the NSAE exon. In some embodiments, the ASO targets a HP1BP3 pre-mRNA sequence comprising an intron flanking the 5′ splice site of the NSAE exon. In some embodiments, the transcript is selected from the group consisting of transcript_ENST00000312239.10, transcript_ENST00000375000.5, transcript_ENST00000375003.6, transcript_ENST00000414993.1, transcript_ENST00000417710.5, transcript_ENST00000419490.5, transcript_ENST00000419948.5, transcript_ENST00000424732.5, transcript_ENST00000437575.5, transcript_ENST00000438032.6, transcript_ENST00000443615.1, transcript_ENST00000487117.1, transcript_ENST00000488722.5, and transcript_ENST00000491748.1.

In some embodiments, the HP1BP3 pre-mRNA transcript is encoded by a genetic sequence with at least about 80%, 85%, 90%, 95%, 96%, 97%, 98%, 99% or 100% sequence identity to the Ensembl reference number ENSG00000127483.19 or a complement thereof. In some embodiments, the HP1BP3 pre-mRNA transcript comprises a sequence with at least about 80%, 85%, 90%, 95%, 96%, 97%, 98%, 99% or 100% sequence identity to a HP1BP3 pre-mRNA transcript or a complement thereof described herein.

In some embodiments, the targeted portion of the HP1BP3 pre-mRNA comprises a sequence with at least 80%, 85%, 90%, 95%, 97%, or 100% sequence identity to a region comprising at least 8 contiguous nucleic acids of a sequence of premrna_ENST00000312239.10, premrna_ENST00000375000.5, premrna_ENST00000375003.6, premrna_ENST00000414993.1, premrna_ENST00000417710.5, premrna_ENST00000419490.5, premrna_ENST00000419948.5, premrna_ENST00000424732.5, premrna_ENST00000437575.5, premrna_ENST00000438032.6, premrna_ENST00000443615.1, premrna_ENST00000487117.1, premrna_ENST00000488722.5, premrna_ENST00000491748.1 or a sequence of Table 3 or complements thereof. In some embodiments, the targeted portion of the HP1BP3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 586-SEQ ID NO: 589 or complements thereof. In some embodiments, the targeted portion of the HP1BP3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a HPS1 genomic sequence. In some embodiments, the ASO targets a NSAE pre-mRNA transcript from a HPS1 genomic sequence comprising a NSAE exon. In some embodiments, the ASO targets a NSAE pre-mRNA transcript from a HPS1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000325103.10, premrna_ENST00000338546.9, premrna_ENST00000359632.7, premrna_ENST00000361490.9, premrna_ENST00000414009.1, premrna_ENST00000462743.1, premrna_ENST00000465957.1, premrna_ENST00000467246.5, premrna_ENST00000470095.5, premrna_ENST00000474873.5, premrna_ENST00000478087.5, premrna_ENST00000480020.5, premrna_ENST00000497527.1, premrna_ENST00000498219.5, and premrna_ENST00000613394.4. In some embodiments, the ASO targets a HPS1 pre-mRNA sequence comprising a NSAE exon. In some embodiments, the ASO targets a HPS1 pre-mRNA sequence comprising an intron flanking the 3′ splice site of the NSAE exon. In some embodiments, the ASO targets a HPS1 pre-mRNA sequence comprising an intron flanking the 5′ splice site of the NSAE exon. In some embodiments, the transcript is selected from the group consisting of transcript_ENST00000325103.10, transcript_ENST00000338546.9, transcript_ENST00000359632.7, transcript_ENST00000361490.9, transcript_ENST00000414009.1, transcript_ENST00000462743.1, transcript_ENST00000465957.1, transcript_ENST00000467246.5, transcript_ENST00000470095.5, transcript_ENST00000474873.5, transcript_ENST00000478087.5, transcript_ENST00000480020.5, transcript_ENST00000497527.1, transcript_ENST00000498219.5, and transcript_ENST00000613394.4.

In some embodiments, the HPS1 pre-mRNA transcript is encoded by a genetic sequence with at least about 80%, 85%, 90%, 95%, 96%, 97%, 98%, 99% or 100% sequence identity to the Ensembl reference number ENSG00000107521.19 or a complement thereof. In some embodiments, the HPS1 pre-mRNA transcript comprises a sequence with at least about 80%, 85%, 90%, 95%, 96%, 97%, 98%, 99% or 100% sequence identity to a HPS1 pre-mRNA transcript or a complement thereof described herein.

In some embodiments, the targeted portion of the HPS1 pre-mRNA comprises a sequence with at least 80%, 85%, 90%, 95%, 97%, or 100% sequence identity to a region comprising at least 8 contiguous nucleic acids of a sequence of premrna_ENST00000325103.10, premrna_ENST00000338546.9, premrna_ENST00000359632.7, premrna_ENST00000361490.9, premrna_ENST00000414009.1, premrna_ENST00000462743.1, premrna_ENST00000465957.1, premrna_ENST00000467246.5, premrna_ENST00000470095.5, premrna_ENST00000474873.5, premrna_ENST00000478087.5, premrna_ENST00000480020.5, premrna_ENST00000497527.1, premrna_ENST00000498219.5, premrna_ENST00000613394.4 or a sequence of Table 3 or complements thereof. In some embodiments, the targeted portion of the HPS1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 590-SEQ ID NO: 593 or complements thereof. In some embodiments, the targeted portion of the HPS1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a HPS4 genomic sequence. In some embodiments, the ASO targets a NSAE pre-mRNA transcript from a HPS4 genomic sequence comprising a NSAE exon. In some embodiments, the ASO targets a NSAE pre-mRNA transcript from a HPS4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000336873.9, premrna_ENST00000398145.6, premrna_ENST00000402105.7, premrna_ENST00000422379.2, premrna_ENST00000429411.5, premrna_ENST00000439453.5, premrna_ENST00000459918.1, premrna_ENST00000464362.5, premrna_ENST00000466781.5, premrna_ENST00000473782.1, premrna_ENST00000479064.5, premrna_ENST00000481910.1, premrna_ENST00000483631.1, premrna_ENST00000485842.5, premrna_ENST00000491142.1, premrna_ENST00000493455.6, premrna_ENST00000496385.5, and premrna_ENST00000519774.5. In some embodiments, the ASO targets a HPS4 pre-mRNA sequence comprising a NSAE exon. In some embodiments, the ASO targets a HPS4 pre-mRNA sequence comprising an intron flanking the 3′ splice site of the NSAE exon. In some embodiments, the ASO targets a HPS4 pre-mRNA sequence comprising an intron flanking the 5′ splice site of the NSAE exon. In some embodiments, the transcript is selected from the group consisting of transcript_ENST00000336873.9, transcript_ENST00000398145.6, transcript_ENST00000402105.7, transcript_ENST00000422379.2, transcript_ENST00000429411.5, transcript_ENST00000439453.5, transcript_ENST00000459918.1, transcript_ENST00000464362.5, transcript_ENST00000466781.5, transcript_ENST00000473782.1, transcript_ENST00000479064.5, transcript_ENST00000481910.1, transcript_ENST00000483631.1, transcript_ENST00000485842.5, transcript_ENST00000491142.1, transcript_ENST00000493455.6, transcript_ENST00000496385.5, and transcript_ENST00000519774.5.

In some embodiments, the HPS4 pre-mRNA transcript is encoded by a genetic sequence with at least about 80%, 85%, 90%, 95%, 96%, 97%, 98%, 99% or 100% sequence identity to the Ensembl reference number ENSG00000100099.20 or a complement thereof. In some embodiments, the HPS4 pre-mRNA transcript comprises a sequence with at least about 80%, 85%, 90%, 95%, 96%, 97%, 98%, 99% or 100% sequence identity to a HPS4 pre-mRNA transcript or a complement thereof described herein.

In some embodiments, the targeted portion of the HPS4 pre-mRNA comprises a sequence with at least 80%, 85%, 90%, 95%, 97%, or 100% sequence identity to a region comprising at least 8 contiguous nucleic acids of a sequence of premrna_ENST00000336873.9, premrna_ENST00000398145.6, premrna_ENST00000402105.7, premrna_ENST00000422379.2, premrna_ENST00000429411.5, premrna_ENST00000439453.5, premrna_ENST00000459918.1, premrna_ENST00000464362.5, premrna_ENST00000466781.5, premrna_ENST00000473782.1, premrna_ENST00000479064.5, premrna_ENST00000481910.1, premrna_ENST00000483631.1, premrna_ENST00000485842.5, premrna_ENST00000491142.1, premrna_ENST00000493455.6, premrna_ENST00000496385.5, premrna_ENST00000519774.5 or a sequence of Table 3 or complements thereof. In some embodiments, the targeted portion of the HPS4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 594-SEQ ID NO: 597 or complements thereof. In some embodiments, the targeted portion of the HPS4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a IDUA genomic sequence. In some embodiments, the ASO targets a NSAE pre-mRNA transcript from a IDUA genomic sequence comprising a NSAE exon. In some embodiments, the ASO targets a NSAE pre-mRNA transcript from a IDUA genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000247933.9, premrna_ENST00000502829.1, premrna_ENST00000502910.5, premrna_ENST00000504568.5, premrna_ENST00000506561.5, premrna_ENST00000508168.5, premrna_ENST00000509744.1, premrna_ENST00000509948.5, premrna_ENST00000514192.5, premrna_ENST00000514224.2, premrna_ENST00000514417.1, premrna_ENST00000514698.5, and premrna_ENST00000652070.1. In some embodiments, the ASO targets a IDUA pre-mRNA sequence comprising a NSAE exon. In some embodiments, the ASO targets a IDUA pre-mRNA sequence comprising an intron flanking the 3′ splice site of the NSAE exon. In some embodiments, the ASO targets a IDUA pre-mRNA sequence comprising an intron flanking the 5′ splice site of the NSAE exon. In some embodiments, the transcript is selected from the group consisting of transcript_ENST00000247933.9, transcript_ENST00000502829.1, transcript_ENST00000502910.5, transcript_ENST00000504568.5, transcript_ENST00000506561.5, transcript_ENST00000508168.5, transcript_ENST00000509744.1, transcript_ENST00000509948.5, transcript_ENST00000514192.5, transcript_ENST00000514224.2, transcript_ENST00000514417.1, transcript_ENST00000514698.5, and transcript_ENST00000652070.1.

In some embodiments, the IDUA pre-mRNA transcript is encoded by a genetic sequence with at least about 80%, 85%, 90%, 95%, 96%, 97%, 98%, 99% or 100% sequence identity to the Ensembl reference number ENSG00000127415.13 or a complement thereof. In some embodiments, the IDUA pre-mRNA transcript comprises a sequence with at least about 80%, 85%, 90%, 95%, 96%, 97%, 98%, 99% or 100% sequence identity to a IDUA pre-mRNA transcript or a complement thereof described herein.

In some embodiments, the targeted portion of the IDUA pre-mRNA comprises a sequence with at least 80%, 85%, 90%, 95%, 97%, or 100% sequence identity to a region comprising at least 8 contiguous nucleic acids of a sequence of premrna_ENST00000247933.9, premrna_ENST00000502829.1, premrna_ENST00000502910.5, premrna_ENST00000504568.5, premrna_ENST00000506561.5, premrna_ENST00000508168.5, premrna_ENST00000509744.1, premrna_ENST00000509948.5, premrna_ENST00000514192.5, premrna_ENST00000514224.2, premrna_ENST00000514417.1, premrna_ENST00000514698.5, premrna_ENST00000652070.1 or a sequence of Table 3 or complements thereof. In some embodiments, the targeted portion of the IDUA pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 598-SEQ ID NO: 603 or complements thereof. In some embodiments, the targeted portion of the IDUA pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a IFT122 genomic sequence. In some embodiments, the ASO targets a NSAE pre-mRNA transcript from a IFT122 genomic sequence comprising a NSAE exon. In some embodiments, the ASO targets a NSAE pre-mRNA transcript from a IFT122 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000296266.7, premrna_ENST00000347300.6, premrna_ENST00000348417.6, premrna_ENST00000349441.6, premrna_ENST00000431818.6, premrna_ENST00000440957.6, premrna_ENST00000448668.2, premrna_ENST00000502304.5, premrna_ENST00000502456.5, premrna_ENST00000504021.5, premrna_ENST00000504444.1, premrna_ENST00000504653.5, premrna_ENST00000506507.5, premrna_ENST00000507221.1, premrna_ENST00000507564.5, premrna_ENST00000508654.1, premrna_ENST00000508826.5, premrna_ENST00000509195.5, premrna_ENST00000509522.5, premrna_ENST00000509815.1, premrna_ENST00000510524.5, premrna_ENST00000511425.5, premrna_ENST00000511498.1, premrna_ENST00000512157.5, premrna_ENST00000512220.5, premrna_ENST00000512814.5, premrna_ENST00000513190.1, premrna_ENST00000513891.1, premrna_ENST00000513932.1, premrna_ENST00000514081.1, premrna_ENST00000514275.5, and premrna_ENST00000515783.5. In some embodiments, the ASO targets a IFT122 pre-mRNA sequence comprising a NSAE exon. In some embodiments, the ASO targets a IFT122 pre-mRNA sequence comprising an intron flanking the 3′ splice site of the NSAE exon. In some embodiments, the ASO targets a IFT122 pre-mRNA sequence comprising an intron flanking the 5′ splice site of the NSAE exon. In some embodiments, the transcript is selected from the group consisting of transcript_ENST00000296266.7, transcript_ENST00000347300.6, transcript_ENST00000348417.6, transcript_ENST00000349441.6, transcript_ENST00000431818.6, transcript_ENST00000440957.6, transcript_ENST00000448668.2, transcript_ENST00000502304.5, transcript_ENST00000502456.5, transcript_ENST00000504021.5, transcript_ENST00000504444.1, transcript_ENST00000504653.5, transcript_ENST00000506507.5, transcript_ENST00000507221.1, transcript_ENST00000507564.5, transcript_ENST00000508654.1, transcript_ENST00000508826.5, transcript_ENST00000509195.5, transcript_ENST00000509522.5, transcript_ENST00000509815.1, transcript_ENST00000510524.5, transcript_ENST00000511425.5, transcript_ENST00000511498.1, transcript_ENST00000512157.5, transcript_ENST00000512220.5, transcript_ENST00000512814.5, transcript_ENST00000513190.1, transcript_ENST00000513891.1, transcript_ENST00000513932.1, transcript_ENST00000514081.1, transcript_ENST00000514275.5, and transcript_ENST00000515783.5.

In some embodiments, the IFT122 pre-mRNA transcript is encoded by a genetic sequence with at least about 80%, 85%, 90%, 95%, 96%, 97%, 98%, 99% or 100% sequence identity to the Ensembl reference number ENSG00000163913.12 or a complement thereof. In some embodiments, the IFT122 pre-mRNA transcript comprises a sequence with at least about 80%, 85%, 90%, 95%, 96%, 97%, 98%, 99% or 100% sequence identity to a IFT122 pre-mRNA transcript or a complement thereof described herein.

In some embodiments, the targeted portion of the IFT122 pre-mRNA comprises a sequence with at least 80%, 85%, 90%, 95%, 97%, or 100% sequence identity to a region comprising at least 8 contiguous nucleic acids of a sequence of premrna_ENST00000296266.7, premrna_ENST00000347300.6, premrna_ENST00000348417.6, premrna_ENST00000349441.6, premrna_ENST00000431818.6, premrna_ENST00000440957.6, premrna_ENST00000448668.2, premrna_ENST00000502304.5, premrna_ENST00000502456.5, premrna_ENST00000504021.5, premrna_ENST00000504444.1, premrna_ENST00000504653.5, premrna_ENST00000506507.5, premrna_ENST00000507221.1, premrna_ENST00000507564.5, premrna_ENST00000508654.1, premrna_ENST00000508826.5, premrna_ENST00000509195.5, premrna_ENST00000509522.5, premrna_ENST00000509815.1, premrna_ENST00000510524.5, premrna_ENST00000511425.5, premrna_ENST00000511498.1, premrna_ENST00000512157.5, premrna_ENST00000512220.5, premrna_ENST00000512814.5, premrna_ENST00000513190.1, premrna_ENST00000513891.1, premrna_ENST00000513932.1, premrna_ENST00000514081.1, premrna_ENST00000514275.5, premrna_ENST00000515783.5 or a sequence of Table 3 or complements thereof. In some embodiments, the targeted portion of the IFT122 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 604-SEQ ID NO: 607 or complements thereof. In some embodiments, the targeted portion of the IFT122 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a IL17RC genomic sequence. In some embodiments, the ASO targets a NSAE pre-mRNA transcript from a IL17RC genomic sequence comprising a NSAE exon. In some embodiments, the ASO targets a NSAE pre-mRNA transcript from a IL17RC genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000295981.7, premrna_ENST00000383812.9, premrna_ENST00000403601.8, premrna_ENST00000412901.5, premrna_ENST00000413608.2, premrna_ENST00000416074.6, premrna_ENST00000424206.5, premrna_ENST00000434756.5, premrna_ENST00000436503.5, premrna_ENST00000438091.5, premrna_ENST00000440502.5, premrna_ENST00000451165.6, premrna_ENST00000451231.5, premrna_ENST00000451271.5, premrna_ENST00000455057.5, premrna_ENST00000461995.5, premrna_ENST00000464406.5, premrna_ENST00000465794.1, premrna_ENST00000466046.5, premrna_ENST00000466712.1, premrna_ENST00000469686.5, premrna_ENST00000476810.5, premrna_ENST00000478206.1, premrna_ENST00000481032.1, premrna_ENST00000483582.5, premrna_ENST00000490512.5, premrna_ENST00000494365.5, premrna_ENST00000497102.5, premrna_ENST00000497387.5, and premrna_ENST00000498214.6. In some embodiments, the ASO targets a IL17RC pre-mRNA sequence comprising a NSAE exon. In some embodiments, the ASO targets a IL17RC pre-mRNA sequence comprising an intron flanking the 3′ splice site of the NSAE exon. In some embodiments, the ASO targets a IL17RC pre-mRNA sequence comprising an intron flanking the 5′ splice site of the NSAE exon. In some embodiments, the transcript is selected from the group consisting of transcript_ENST00000295981.7, transcript_ENST00000383812.9, transcript_ENST00000403601.8, transcript_ENST00000412901.5, transcript_ENST00000413608.2, transcript_ENST00000416074.6, transcript_ENST00000424206.5, transcript_ENST00000434756.5, transcript_ENST00000436503.5, transcript_ENST00000438091.5, transcript_ENST00000440502.5, transcript_ENST00000451165.6, transcript_ENST00000451231.5, transcript_ENST00000451271.5, transcript_ENST00000455057.5, transcript_ENST00000461995.5, transcript_ENST00000464406.5, transcript_ENST00000465794.1, transcript_ENST00000466046.5, transcript_ENST00000466712.1, transcript_ENST00000469686.5, transcript_ENST00000476810.5, transcript_ENST00000478206.1, transcript_ENST00000481032.1, transcript_ENST00000483582.5, transcript_ENST00000490512.5, transcript_ENST00000494365.5, transcript_ENST00000497102.5, transcript_ENST00000497387.5, and transcript_ENST00000498214.6.

In some embodiments, the IL17RC pre-mRNA transcript is encoded by a genetic sequence with at least about 80%, 85%, 90%, 95%, 96%, 97%, 98%, 99% or 100% sequence identity to the Ensembl reference number ENSG00000163702.20 or a complement thereof. In some embodiments, the IL17RC pre-mRNA transcript comprises a sequence with at least about 80%, 85%, 90%, 95%, 96%, 97%, 98%, 99% or 100% sequence identity to a IL17RC pre-mRNA transcript or a complement thereof described herein.

In some embodiments, the targeted portion of the IL17RC pre-mRNA comprises a sequence with at least 80%, 85%, 90%, 95%, 97%, or 100% sequence identity to a region comprising at least 8 contiguous nucleic acids of a sequence of premrna_ENST00000295981.7, premrna_ENST00000383812.9, premrna_ENST00000403601.8, premrna_ENST00000412901.5, premrna_ENST00000413608.2, premrna_ENST00000416074.6, premrna_ENST00000424206.5, premrna_ENST00000434756.5, premrna_ENST00000436503.5, premrna_ENST00000438091.5, premrna_ENST00000440502.5, premrna_ENST00000451165.6, premrna_ENST00000451231.5, premrna_ENST00000451271.5, premrna_ENST00000455057.5, premrna_ENST00000461995.5, premrna_ENST00000464406.5, premrna_ENST00000465794.1, premrna_ENST00000466046.5, premrna_ENST00000466712.1, premrna_ENST00000469686.5, premrna_ENST00000476810.5, premrna_ENST00000478206.1, premrna_ENST00000481032.1, premrna_ENST00000483582.5, premrna_ENST00000490512.5, premrna_ENST00000494365.5, premrna_ENST00000497102.5, premrna_ENST00000497387.5, premrna_ENST00000498214.6 or a sequence of Table 3 or complements thereof. In some embodiments, the targeted portion of the IL17RC pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 608-SEQ ID NO: 611 or complements thereof. In some embodiments, the targeted portion of the IL17RC pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a IMPDH1 genomic sequence. In some embodiments, the ASO targets a NSAE pre-mRNA transcript from a IMPDH1 genomic sequence comprising a NSAE exon. In some embodiments, the ASO targets a NSAE pre-mRNA transcript from a IMPDH1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000338791.11, premrna_ENST00000348127.10, premrna_ENST00000354269.9, premrna_ENST00000419067.6, premrna_ENST00000460045.1, premrna_ENST00000468842.1, premrna_ENST00000469328.5, premrna_ENST00000470772.5, premrna_ENST00000473463.1, premrna_ENST00000480861.5, premrna_ENST00000484496.5, premrna_ENST00000489263.1, premrna_ENST00000491376.5, premrna_ENST00000496200.5, premrna_ENST00000496487.5, premrna_ENST00000497868.5, premrna_ENST00000626419.2, and premrna_ENST00000648462.1. In some embodiments, the ASO targets a IMPDH1 pre-mRNA sequence comprising a NSAE exon. In some embodiments, the ASO targets a IMPDH1 pre-mRNA sequence comprising an intron flanking the 3′ splice site of the NSAE exon. In some embodiments, the ASO targets a IMPDH1 pre-mRNA sequence comprising an intron flanking the 5′ splice site of the NSAE exon. In some embodiments, the transcript is selected from the group consisting of transcript_ENST00000338791.11, transcript_ENST00000348127.10, transcript_ENST00000354269.9, transcript_ENST00000419067.6, transcript_ENST00000460045.1, transcript_ENST00000468842.1, transcript_ENST00000469328.5, transcript_ENST00000470772.5, transcript_ENST00000473463.1, transcript_ENST00000480861.5, transcript_ENST00000484496.5, transcript_ENST00000489263.1, transcript_ENST00000491376.5, transcript_ENST00000496200.5, transcript_ENST00000496487.5, transcript_ENST00000497868.5, transcript_ENST00000626419.2, and transcript_ENST00000648462.1.

In some embodiments, the IMPDH1 pre-mRNA transcript is encoded by a genetic sequence with at least about 80%, 85%, 90%, 95%, 96%, 97%, 98%, 99% or 100% sequence identity to the Ensembl reference number ENSG00000106348.18 or a complement thereof. In some embodiments, the IMPDH1 pre-mRNA transcript comprises a sequence with at least about 80%, 85%, 90%, 95%, 96%, 97%, 98%, 99% or 100% sequence identity to a IMPDH1 pre-mRNA transcript or a complement thereof described herein.

In some embodiments, the targeted portion of the IMPDH1 pre-mRNA comprises a sequence with at least 80%, 85%, 90%, 95%, 97%, or 100% sequence identity to a region comprising at least 8 contiguous nucleic acids of a sequence of premrna_ENST00000338791.11, premrna_ENST00000348127.10, premrna_ENST00000354269.9, premrna_ENST00000419067.6, premrna_ENST00000460045.1, premrna_ENST00000468842.1, premrna_ENST00000469328.5, premrna_ENST00000470772.5, premrna_ENST00000473463.1, premrna_ENST00000480861.5, premrna_ENST00000484496.5, premrna_ENST00000489263.1, premrna_ENST00000491376.5, premrna_ENST00000496200.5, premrna_ENST00000496487.5, premrna_ENST00000497868.5, premrna_ENST00000626419.2, premrna_ENST00000648462.1 or a sequence of Table 3 or complements thereof. In some embodiments, the targeted portion of the IMPDH1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 612-SEQ ID NO: 617 or complements thereof. In some embodiments, the targeted portion of the IMPDH1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a JNVS genomic sequence. In some embodiments, the ASO targets a NSAE pre-mRNA transcript from a INVS genomic sequence comprising a NSAE exon. In some embodiments, the ASO targets a NSAE pre-mRNA transcript from a INVS genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000262456.6, premrna_ENST00000262457.7, premrna_ENST00000374921.3, premrna_ENST00000460636.2, premrna_ENST00000466647.5, premrna_ENST00000480309.1, and premrna_ENST00000496467.5. In some embodiments, the ASO targets a INVS pre-mRNA sequence comprising a NSAE exon. In some embodiments, the ASO targets a INVS pre-mRNA sequence comprising an intron flanking the 3′ splice site of the NSAE exon. In some embodiments, the ASO targets a INVS pre-mRNA sequence comprising an intron flanking the 5′ splice site of the NSAE exon. In some embodiments, the transcript is selected from the group consisting of transcript_ENST00000262456.6, transcript_ENST00000262457.7, transcript_ENST00000374921.3, transcript_ENST00000460636.2, transcript_ENST00000466647.5, transcript_ENST00000480309.1, and transcript_ENST00000496467.5.

In some embodiments, the INVS pre-mRNA transcript is encoded by a genetic sequence with at least about 80%, 85%, 90%, 95%, 96%, 97%, 98%, 99% or 100% sequence identity to the Ensembl reference number ENSG00000119509.13 or a complement thereof. In some embodiments, the INVS pre-mRNA transcript comprises a sequence with at least about 80%, 85%, 90%, 95%, 96%, 97%, 98%, 99% or 100% sequence identity to a INVS pre-mRNA transcript or a complement thereof described herein.

In some embodiments, the targeted portion of the INVS pre-mRNA comprises a sequence with at least 80%, 85%, 90%, 95%, 97%, or 100% sequence identity to a region comprising at least 8 contiguous nucleic acids of a sequence of premrna_ENST00000262456.6, premrna_ENST00000262457.7, premrna_ENST00000374921.3, premrna_ENST00000460636.2, premrna_ENST00000466647.5, premrna_ENST00000480309.1, premrna_ENST00000496467.5 or a sequence of Table 3 or complements thereof. In some embodiments, the targeted portion of the INVS pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 618-SEQ ID NO: 621 or complements thereof. In some embodiments, the targeted portion of the INVS pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a IRF3 genomic sequence. In some embodiments, the ASO targets a NSAE pre-mRNA transcript from a IRF3 genomic sequence comprising a NSAE exon. In some embodiments, the ASO targets a NSAE pre-mRNA transcript from a IRF3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000309877.11, premrna_ENST00000377135.8, premrna_ENST00000377139.8, premrna_ENST00000442265.2, premrna_ENST00000593337.5, premrna_ENST00000593818.5, premrna_ENST00000593922.5, premrna_ENST00000594387.1, premrna_ENST00000595034.5, premrna_ENST00000595240.5, premrna_ENST00000596644.5, premrna_ENST00000596756.5, premrna_ENST00000596765.5, premrna_ENST00000596788.1, premrna_ENST00000596822.5, premrna_ENST00000597180.1, premrna_ENST00000597198.5, premrna_ENST00000597369.1, premrna_ENST00000597636.5, premrna_ENST00000598108.5, premrna_ENST00000598808.5, premrna_ENST00000599144.5, premrna_ENST00000599223.5, premrna_ENST00000599680.1, premrna_ENST00000600022.5, premrna_ENST00000600453.1, premrna_ENST00000600911.5, premrna_ENST00000601291.5, premrna_ENST00000601373.5, premrna_ENST00000601809.5, and premrna_ENST00000602190.1. In some embodiments, the ASO targets a IRF3 pre-mRNA sequence comprising a NSAE exon. In some embodiments, the ASO targets a IRF3 pre-mRNA sequence comprising an intron flanking the 3′ splice site of the NSAE exon. In some embodiments, the ASO targets a IRF3 pre-mRNA sequence comprising an intron flanking the 5′ splice site of the NSAE exon. In some embodiments, the transcript is selected from the group consisting of transcript_ENST00000309877.11, transcript_ENST00000377135.8, transcript_ENST00000377139.8, transcript_ENST00000442265.2, transcript_ENST00000593337.5, transcript_ENST00000593818.5, transcript_ENST00000593922.5, transcript_ENST00000594387.1, transcript_ENST00000595034.5, transcript_ENST00000595240.5, transcript_ENST00000596644.5, transcript_ENST00000596756.5, transcript_ENST00000596765.5, transcript_ENST00000596788.1, transcript_ENST00000596822.5, transcript_ENST00000597180.1, transcript_ENST00000597198.5, transcript_ENST00000597369.1, transcript_ENST00000597636.5, transcript_ENST00000598108.5, transcript_ENST00000598808.5, transcript_ENST00000599144.5, transcript_ENST00000599223.5, transcript_ENST00000599680.1, transcript_ENST00000600022.5, transcript_ENST00000600453.1, transcript_ENST00000600911.5, transcript_ENST00000601291.5, transcript_ENST00000601373.5, transcript_ENST00000601809.5, and transcript_ENST00000602190.1.

In some embodiments, the IRF3 pre-mRNA transcript is encoded by a genetic sequence with at least about 80%, 85%, 90%, 95%, 96%, 97%, 98%, 99% or 100% sequence identity to the Ensembl reference number ENSG00000126456.16 or a complement thereof. In some embodiments, the IRF3 pre-mRNA transcript comprises a sequence with at least about 80%, 85%, 90%, 95%, 96%, 97%, 98%, 99% or 100% sequence identity to a IRF3 pre-mRNA transcript or a complement thereof described herein.

In some embodiments, the targeted portion of the IRF3 pre-mRNA comprises a sequence with at least 80%, 85%, 90%, 95%, 97%, or 100% sequence identity to a region comprising at least 8 contiguous nucleic acids of a sequence of premrna_ENST00000309877.11, premrna_ENST00000377135.8, premrna_ENST00000377139.8, premrna_ENST00000442265.2, premrna_ENST00000593337.5, premrna_ENST00000593818.5, premrna_ENST00000593922.5, premrna_ENST00000594387.1, premrna_ENST00000595034.5, premrna_ENST00000595240.5, premrna_ENST00000596644.5, premrna_ENST00000596756.5, premrna_ENST00000596765.5, premrna_ENST00000596788.1, premrna_ENST00000596822.5, premrna_ENST00000597180.1, premrna_ENST00000597198.5, premrna_ENST00000597369.1, premrna_ENST00000597636.5, premrna_ENST00000598108.5, premrna_ENST00000598808.5, premrna_ENST00000599144.5, premrna_ENST00000599223.5, premrna_ENST00000599680.1, premrna_ENST00000600022.5, premrna_ENST00000600453.1, premrna_ENST00000600911.5, premrna_ENST00000601291.5, premrna_ENST00000601373.5, premrna_ENST00000601809.5, premrna_ENST00000602190.1 or a sequence of Table 3 or complements thereof. In some embodiments, the targeted portion of the IRF3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 622-SEQ ID NO: 627 or complements thereof. In some embodiments, the targeted portion of the IRF3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a KCTD3 genomic sequence. In some embodiments, the ASO targets a NSAE pre-mRNA transcript from a KCTD3 genomic sequence comprising a NSAE exon. In some embodiments, the ASO targets a NSAE pre-mRNA transcript from a KCTD3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000259154.9, premrna_ENST00000448333.1, premrna_ENST00000452413.1, premrna_ENST00000465650.1, and premrna_ENST00000495537.1. In some embodiments, the ASO targets a KCTD3 pre-mRNA sequence comprising a NSAE exon. In some embodiments, the ASO targets a KCTD3 pre-mRNA sequence comprising an intron flanking the 3′ splice site of the NSAE exon. In some embodiments, the ASO targets a KCTD3 pre-mRNA sequence comprising an intron flanking the 5′ splice site of the NSAE exon. In some embodiments, the transcript is selected from the group consisting of transcript_ENST00000259154.9, transcript_ENST00000448333.1, transcript_ENST00000452413.1, transcript_ENST00000465650.1, and transcript_ENST00000495537.1.

In some embodiments, the KCTD3 pre-mRNA transcript is encoded by a genetic sequence with at least about 80%, 85%, 90%, 95%, 96%, 97%, 98%, 99% or 100% sequence identity to the Ensembl reference number ENSG00000136636.13 or a complement thereof. In some embodiments, the KCTD3 pre-mRNA transcript comprises a sequence with at least about 80%, 85%, 90%, 95%, 96%, 97%, 98%, 99% or 100% sequence identity to a KCTD3 pre-mRNA transcript or a complement thereof described herein.

In some embodiments, the targeted portion of the KCTD3 pre-mRNA comprises a sequence with at least 80%, 85%, 90%, 95%, 97%, or 100% sequence identity to a region comprising at least 8 contiguous nucleic acids of a sequence of premrna_ENST00000259154.9, premrna_ENST00000448333.1, premrna_ENST00000452413.1, premrna_ENST00000465650.1, premrna_ENST00000495537.1 or a sequence of Table 3 or complements thereof. In some embodiments, the targeted portion of the KCTD3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 628-SEQ ID NO: 631 or complements thereof. In some embodiments, the targeted portion of the KCTD3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a KLKB1 genomic sequence. In some embodiments, the ASO targets a NSAE pre-mRNA transcript from a KLKB1 genomic sequence comprising a NSAE exon. In some embodiments, the ASO targets a NSAE pre-mRNA transcript from a KLKB1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000264690.11, premrna_ENST00000428196.5, premrna_ENST00000446598.6, premrna_ENST00000467271.1, premrna_ENST00000511406.5, premma_ENST00000511608.5, and premrna_ENST00000513864.2. In some embodiments, the ASO targets a KLKB1 pre-mRNA sequence comprising a NSAE exon. In some embodiments, the ASO targets a KLKB1 pre-mRNA sequence comprising an intron flanking the 3′ splice site of the NSAE exon. In some embodiments, the ASO targets a KLKB1 pre-mRNA sequence comprising an intron flanking the 5′ splice site of the NSAE exon. In some embodiments, the transcript is selected from the group consisting of transcript_ENST00000264690.11, transcript_ENST00000428196.5, transcript_ENST00000446598.6, transcript_ENST00000467271.1, transcript_ENST00000511406.5, transcript_ENST00000511608.5, and transcript_ENST00000513864.2.

In some embodiments, the KLKB1 pre-mRNA transcript is encoded by a genetic sequence with at least about 80%, 85%, 90%, 95%, 96%, 97%, 98%, 99% or 100% sequence identity to the Ensembl reference number ENSG00000164344.16 or a complement thereof. In some embodiments, the KLKB1 pre-mRNA transcript comprises a sequence with at least about 80%, 85%, 90%, 95%, 96%, 97%, 98%, 99% or 100% sequence identity to a KLKB1 pre-mRNA transcript or a complement thereof described herein.

In some embodiments, the targeted portion of the KLKB1 pre-mRNA comprises a sequence with at least 80%, 85%, 90%, 95%, 97%, or 100% sequence identity to a region comprising at least 8 contiguous nucleic acids of a sequence of premrna_ENST00000264690.11, premrna_ENST00000428196.5, premrna_ENST00000446598.6, premrna_ENST00000467271.1, premrna_ENST00000511406.5, premrna_ENST00000511608.5, premrna_ENST00000513864.2 or a sequence of Table 3 or complements thereof. In some embodiments, the targeted portion of the KLKB1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 632-SEQ ID NO: 635 or complements thereof. In some embodiments, the targeted portion of the KLKB1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a KYAT1 genomic sequence. In some embodiments, the ASO targets a NSAE pre-mRNA transcript from a KYAT1 genomic sequence comprising a NSAE exon. In some embodiments, the ASO targets a NSAE pre-mRNA transcript from a KYAT1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000302586.8, premrna_ENST00000320665.10, premrna_ENST00000416084.5, premrna_ENST00000427720.1, premrna_ENST00000436267.7, premrna_ENST00000451800.5, premrna_ENST00000462722.5, premrna_ENST00000466418.1, premrna_ENST00000474824.1, and premrna_ENST00000483599.5. In some embodiments, the ASO targets a KYAT1 pre-mRNA sequence comprising a NSAE exon. In some embodiments, the ASO targets a KYAT1 pre-mRNA sequence comprising an intron flanking the 3′ splice site of the NSAE exon. In some embodiments, the ASO targets a KYAT1 pre-mRNA sequence comprising an intron flanking the 5′ splice site of the NSAE exon. In some embodiments, the transcript is selected from the group consisting of transcript_ENST00000302586.8, transcript_ENST00000320665.10, transcript_ENST00000416084.5, transcript_ENST00000427720.1, transcript_ENST00000436267.7, transcript_ENST00000451800.5, transcript_ENST00000462722.5, transcript_ENST00000466418.1, transcript_ENST00000474824.1, and transcript_ENST00000483599.5.

In some embodiments, the KYAT1 pre-mRNA transcript is encoded by a genetic sequence with at least about 80%, 85%, 90%, 95%, 96%, 97%, 98%, 99% or 100% sequence identity to the Ensembl reference number ENSG00000171097.14 or a complement thereof. In some embodiments, the KYAT1 pre-mRNA transcript comprises a sequence with at least about 80%, 85%, 90%, 95%, 96%, 97%, 98%, 99% or 100% sequence identity to a KYAT1 pre-mRNA transcript or a complement thereof described herein.

In some embodiments, the targeted portion of the KYAT1 pre-mRNA comprises a sequence with at least 80%, 85%, 90%, 95%, 97%, or 100% sequence identity to a region comprising at least 8 contiguous nucleic acids of a sequence of premrna_ENST00000302586.8, premrna_ENST00000320665.10, premrna_ENST00000416084.5, premrna_ENST00000427720.1, premrna_ENST00000436267.7, premrna_ENST00000451800.5, premrna_ENST00000462722.5, premrna_ENST00000466418.1, premrna_ENST00000474824.1, premrna_ENST00000483599.5 or a sequence of Table 3 or complements thereof. In some embodiments, the targeted portion of the KYAT1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 636-SEQ ID NO: 641 or complements thereof. In some embodiments, the targeted portion of the KYAT1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a LAMC3 genomic sequence. In some embodiments, the ASO targets a NSAE pre-mRNA transcript from a LAMC3 genomic sequence comprising a NSAE exon. In some embodiments, the ASO targets a NSAE pre-mRNA transcript from a LAMC3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000355452.5, premrna_ENST00000361069.9, premrna_ENST00000462567.1, and premrna_ENST00000480883.1. In some embodiments, the ASO targets a LAMC3 pre-mRNA sequence comprising a NSAE exon. In some embodiments, the ASO targets a LAMC3 pre-mRNA sequence comprising an intron flanking the 3′ splice site of the NSAE exon. In some embodiments, the ASO targets a LAMC3 pre-mRNA sequence comprising an intron flanking the 5′ splice site of the NSAE exon. In some embodiments, the transcript is selected from the group consisting of transcript_ENST00000355452.5, transcript_ENST00000361069.9, transcript_ENST00000462567.1, and transcript_ENST00000480883.1.

In some embodiments, the LAMC3 pre-mRNA transcript is encoded by a genetic sequence with at least about 80%, 85%, 90%, 95%, 96%, 97%, 98%, 99% or 100% sequence identity to the Ensembl reference number ENSG00000050555.18 or a complement thereof. In some embodiments, the LAMC3 pre-mRNA transcript comprises a sequence with at least about 80%, 85%, 90%, 95%, 96%, 97%, 98%, 99% or 100% sequence identity to a LAMC3 pre-mRNA transcript or a complement thereof described herein.

In some embodiments, the targeted portion of the LAMC3 pre-mRNA comprises a sequence with at least 80%, 85%, 90%, 95%, 97%, or 100% sequence identity to a region comprising at least 8 contiguous nucleic acids of a sequence of premrna_ENST00000355452.5, premrna_ENST00000361069.9, premrna_ENST00000462567.1, premrna_ENST00000480883.1 or a sequence of Table 3 or complements thereof. In some embodiments, the targeted portion of the LAMC3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 642-SEQ ID NO: 647 or complements thereof. In some embodiments, the targeted portion of the LAMC3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a LDAH genomic sequence. In some embodiments, the ASO targets a NSAE pre-mRNA transcript from a LDAH genomic sequence comprising a NSAE exon. In some embodiments, the ASO targets a NSAE pre-mRNA transcript from a LDAH genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000237822.8, premrna_ENST00000381090.7, premrna_ENST00000402479.6, premrna_ENST00000403006.6, premrna_ENST00000412261.5, premrna_ENST00000419825.2, premrna_ENST00000432947.1, premrna_ENST00000435420.6, premrna_ENST00000440866.6, premrna_ENST00000470099.1, premrna_ENST00000541941.5, premrna_ENST00000619656.4, and premrna_ENST00000626491.2. In some embodiments, the ASO targets a LDAH pre-mRNA sequence comprising a NSAE exon. In some embodiments, the ASO targets a LDAH pre-mRNA sequence comprising an intron flanking the 3′ splice site of the NSAE exon. In some embodiments, the ASO targets a LDAH pre-mRNA sequence comprising an intron flanking the 5′ splice site of the NSAE exon. In some embodiments, the transcript is selected from the group consisting of transcript_ENST00000237822.8, transcript_ENST00000381090.7, transcript_ENST00000402479.6, transcript_ENST00000403006.6, transcript_ENST00000412261.5, transcript_ENST00000419825.2, transcript_ENST00000432947.1, transcript_ENST00000435420.6, transcript_ENST00000440866.6, transcript_ENST00000470099.1, transcript_ENST00000541941.5, transcript_ENST00000619656.4, and transcript_ENST00000626491.2.

In some embodiments, the LDAH pre-mRNA transcript is encoded by a genetic sequence with at least about 80%, 85%, 90%, 95%, 96%, 97%, 98%, 99% or 100% sequence identity to the Ensembl reference number ENSG00000118961.15 or a complement thereof. In some embodiments, the LDAH pre-mRNA transcript comprises a sequence with at least about 80%, 85%, 90%, 95%, 96%, 97%, 98%, 99% or 100% sequence identity to a LDAH pre-mRNA transcript or a complement thereof described herein.

In some embodiments, the targeted portion of the LDAH pre-mRNA comprises a sequence with at least 80%, 85%, 90%, 95%, 97%, or 100% sequence identity to a region comprising at least 8 contiguous nucleic acids of a sequence of premrna_ENST00000237822.8, premrna_ENST00000381090.7, premrna_ENST00000402479.6, premrna_ENST00000403006.6, premrna_ENST00000412261.5, premrna_ENST00000419825.2, premrna_ENST00000432947.1, premrna_ENST00000435420.6, premrna_ENST00000440866.6, premrna_ENST00000470099.1, premrna_ENST00000541941.5, premrna_ENST00000619656.4, premrna_ENST00000626491.2 or a sequence of Table 3 or complements thereof. In some embodiments, the targeted portion of the LDAH pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 648-SEQ ID NO: 651 or complements thereof. In some embodiments, the targeted portion of the LDAH pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a LIMS2 genomic sequence. In some embodiments, the ASO targets a NSAE pre-mRNA transcript from a LIMS2 genomic sequence comprising a NSAE exon. In some embodiments, the ASO targets a NSAE pre-mRNA transcript from a LIMS2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000324938.9, premrna_ENST00000355119.8, premrna_ENST00000409254.1, premrna_ENST00000409286.5, premrna_ENST00000409455.5, premrna_ENST00000409754.5, premrna_ENST00000409808.6, premrna_ENST00000410011.5, premrna_ENST00000410038.5, premrna_ENST00000413578.5, premrna_ENST00000426981.5, premrna_ENST00000466410.5, premrna_ENST00000469300.6, premrna_ENST00000476932.5, premrna_ENST00000484252.5, premrna_ENST00000494613.5, premrna_ENST00000545738.6, and premrna_ENST00000582671.1. In some embodiments, the ASO targets a LIMS2 pre-mRNA sequence comprising a NSAE exon. In some embodiments, the ASO targets a LIMS2 pre-mRNA sequence comprising an intron flanking the 3′ splice site of the NSAE exon. In some embodiments, the ASO targets a LIMS2 pre-mRNA sequence comprising an intron flanking the 5′ splice site of the NSAE exon. In some embodiments, the transcript is selected from the group consisting of transcript_ENST00000324938.9, transcript_ENST00000355119.8, transcript_ENST00000409254.1, transcript_ENST00000409286.5, transcript_ENST00000409455.5, transcript_ENST00000409754.5, transcript_ENST00000409808.6, transcript_ENST00000410011.5, transcript_ENST00000410038.5, transcript_ENST00000413578.5, transcript_ENST00000426981.5, transcript_ENST00000466410.5, transcript_ENST00000469300.6, transcript_ENST00000476932.5, transcript_ENST00000484252.5, transcript_ENST00000494613.5, transcript_ENST00000545738.6, and transcript_ENST00000582671.1.

In some embodiments, the LIMS2 pre-mRNA transcript is encoded by a genetic sequence with at least about 80%, 85%, 90%, 95%, 96%, 97%, 98%, 99% or 100% sequence identity to the Ensembl reference number ENSG00000072163.19 or a complement thereof. In some embodiments, the LIMS2 pre-mRNA transcript comprises a sequence with at least about 80%, 85%, 90%, 95%, 96%, 97%, 98%, 99% or 100% sequence identity to a LIMS2 pre-mRNA transcript or a complement thereof described herein.

In some embodiments, the targeted portion of the LIMS2 pre-mRNA comprises a sequence with at least 80%, 85%, 90%, 95%, 97%, or 100% sequence identity to a region comprising at least 8 contiguous nucleic acids of a sequence of premrna_ENST00000324938.9, premrna_ENST00000355119.8, premrna_ENST00000409254.1, premrna_ENST00000409286.5, premrna_ENST00000409455.5, premrna_ENST00000409754.5, premrna_ENST00000409808.6, premrna_ENST00000410011.5, premrna_ENST00000410038.5, premrna_ENST00000413578.5, premrna_ENST00000426981.5, premrna_ENST00000466410.5, premrna_ENST00000469300.6, premrna_ENST00000476932.5, premrna_ENST00000484252.5, premrna_ENST00000494613.5, premrna_ENST00000545738.6, premrna_ENST00000582671.1 or a sequence of Table 3 or complements thereof. In some embodiments, the targeted portion of the LIMS2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 652-SEQ ID NO: 659 or complements thereof. In some embodiments, the targeted portion of the LIMS2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a MALT1 genomic sequence. In some embodiments, the ASO targets a NSAE pre-mRNA transcript from a MALT1 genomic sequence comprising a NSAE exon. In some embodiments, the ASO targets a NSAE pre-mRNA transcript from a MALT1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000345724.7, premrna_ENST00000587438.1, premrna_ENST00000589873.5, premrna_ENST00000591792.1, premrna_ENST00000648670.1, premrna_ENST00000649125.1, premrna_ENST00000649202.1, premrna_ENST00000649217.2, premrna_ENST00000649756.1, premrna_ENST00000650045.1, and premrna_ENST00000650355.1. In some embodiments, the ASO targets a MALT1 pre-mRNA sequence comprising a NSAE exon. In some embodiments, the ASO targets a MALT1 pre-mRNA sequence comprising an intron flanking the 3′ splice site of the NSAE exon. In some embodiments, the ASO targets a MALT1 pre-mRNA sequence comprising an intron flanking the 5′ splice site of the NSAE exon. In some embodiments, the transcript is selected from the group consisting of transcript_ENST00000345724.7, transcript_ENST00000587438.1, transcript_ENST00000589873.5, transcript_ENST00000591792.1, transcript_ENST00000648670.1, transcript_ENST00000649125.1, transcript_ENST00000649202.1, transcript_ENST00000649217.2, transcript_ENST00000649756.1, transcript_ENST00000650045.1, and transcript_ENST00000650355.1.

In some embodiments, the MALT1 pre-mRNA transcript is encoded by a genetic sequence with at least about 80%, 85%, 90%, 95%, 96%, 97%, 98%, 99% or 100% sequence identity to the Ensembl reference number ENSG00000172175.15 or a complement thereof. In some embodiments, the MALT1 pre-mRNA transcript comprises a sequence with at least about 80%, 85%, 90%, 95%, 96%, 97%, 98%, 99% or 100% sequence identity to a MALT1 pre-mRNA transcript or a complement thereof described herein.

In some embodiments, the targeted portion of the MALT1 pre-mRNA comprises a sequence with at least 80%, 85%, 90%, 95%, 97%, or 100% sequence identity to a region comprising at least 8 contiguous nucleic acids of a sequence of premrna_ENST00000345724.7, premrna_ENST00000587438.1, premrna_ENST00000589873.5, premrna_ENST00000591792.1, premrna_ENST00000648670.1, premrna_ENST00000649125.1, premrna_ENST00000649202.1, premrna_ENST00000649217.2, premrna_ENST00000649756.1, premrna_ENST00000650045.1, premrna_ENST00000650355.1, or a sequence of Table 3 or complements thereof. In some embodiments, the targeted portion of the MALT1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 660-SEQ ID NO: 663 or complements thereof. In some embodiments, the targeted portion of the MALT1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a MAP3K7 genomic sequence. In some embodiments, the ASO targets a NSAE pre-mRNA transcript from a MAP3K7 genomic sequence comprising a NSAE exon. In some embodiments, the ASO targets a NSAE pre-mRNA transcript from a MAP3K7 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000369320.1, premrna_ENST00000369325.7, premrna_ENST00000369327.7, premrna_ENST00000369329.8, premrna_ENST00000369332.7, and premrna_ENST00000479630.1. In some embodiments, the ASO targets a MAP3K7 pre-mRNA sequence comprising a NSAE exon. In some embodiments, the ASO targets a MAP3K7 pre-mRNA sequence comprising an intron flanking the 3′ splice site of the NSAE exon. In some embodiments, the ASO targets a MAP3K7 pre-mRNA sequence comprising an intron flanking the 5′ splice site of the NSAE exon. In some embodiments, the transcript is selected from the group consisting of transcript_ENST00000369320.1, transcript_ENST00000369325.7, transcript_ENST00000369327.7, transcript_ENST00000369329.8, transcript_ENST00000369332.7, and transcript_ENST00000479630.1.

In some embodiments, the MAP3K7 pre-mRNA transcript is encoded by a genetic sequence with at least about 80%, 85%, 90%, 95%, 96%, 97%, 98%, 99% or 100% sequence identity to the Ensembl reference number ENSG00000135341.18 or a complement thereof. In some embodiments, the MAP3K7 pre-mRNA transcript comprises a sequence with at least about 80%, 85%, 90%, 95%, 96%, 97%, 98%, 99% or 100% sequence identity to a MAP3K7 pre-mRNA transcript or a complement thereof described herein.

In some embodiments, the targeted portion of the MAP3K7 pre-mRNA comprises a sequence with at least 80%, 85%, 90%, 95%, 97%, or 100% sequence identity to a region comprising at least 8 contiguous nucleic acids of a sequence of premrna_ENST00000369320.1, premrna_ENST00000369325.7, premrna_ENST00000369327.7, premrna_ENST00000369329.8, premrna_ENST00000369332.7, premrna_ENST00000479630.1 or a sequence of Table 3 or complements thereof. In some embodiments, the targeted portion of the MAP3K7 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 664-SEQ ID NO: 667 or complements thereof. In some embodiments, the targeted portion of the MAP3K7 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a MAPK13 genomic sequence. In some embodiments, the ASO targets a NSAE pre-mRNA transcript from a MAPK13 genomic sequence comprising a NSAE exon. In some embodiments, the ASO targets a NSAE pre-mRNA transcript from a MAPK13 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000211287.9, premrna_ENST00000373759.1, premrna_ENST00000373766.9, premrna_ENST00000476951.5, and premrna_ENST00000490334.1. In some embodiments, the ASO targets a MAPK13 pre-mRNA sequence comprising a NSAE exon. In some embodiments, the ASO targets a MAPK13 pre-mRNA sequence comprising an intron flanking the 3′ splice site of the NSAE exon. In some embodiments, the ASO targets a MAPK13 pre-mRNA sequence comprising an intron flanking the 5′ splice site of the NSAE exon. In some embodiments, the transcript is selected from the group consisting of transcript_ENST00000211287.9, transcript_ENST00000373759.1, transcript_ENST00000373766.9, transcript_ENST00000476951.5, and transcript_ENST00000490334.1.

In some embodiments, the MAPK13 pre-mRNA transcript is encoded by a genetic sequence with at least about 80%, 85%, 90%, 95%, 96%, 97%, 98%, 99% or 100% sequence identity to the Ensembl reference number ENSG00000156711.17 or a complement thereof. In some embodiments, the MAPK13 pre-mRNA transcript comprises a sequence with at least about 80%, 85%, 90%, 95%, 96%, 97%, 98%, 99% or 100% sequence identity to a MAPK13 pre-mRNA transcript or a complement thereof described herein.

In some embodiments, the targeted portion of the MAPK13 pre-mRNA comprises a sequence with at least 80%, 85%, 90%, 95%, 97%, or 100% sequence identity to a region comprising at least 8 contiguous nucleic acids of a sequence of premrna_ENST00000211287.9, premrna_ENST00000373759.1, premrna_ENST00000373766.9, premrna_ENST00000476951.5, premrna_ENST00000490334.1 or a sequence of Table 3 or complements thereof. In some embodiments, the targeted portion of the MAPK13 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 668-SEQ ID NO: 675 or complements thereof. In some embodiments, the targeted portion of the MAPK13 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a MCAT genomic sequence. In some embodiments, the ASO targets a NSAE pre-mRNA transcript from a MCAT genomic sequence comprising a NSAE exon. In some embodiments, the ASO targets a NSAE pre-mRNA transcript from a MCAT genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000290429.11, premrna_ENST00000327555.5, premrna_ENST00000464244.1, and premrna_ENST00000608052.1. In some embodiments, the ASO targets a MCAT pre-mRNA sequence comprising a NSAE exon. In some embodiments, the ASO targets a MCAT pre-mRNA sequence comprising an intron flanking the 3′ splice site of the NSAE exon. In some embodiments, the ASO targets a MCAT pre-mRNA sequence comprising an intron flanking the 5′ splice site of the NSAE exon. In some embodiments, the transcript is selected from the group consisting of transcript_ENST00000290429.11, transcript_ENST00000327555.5, transcript_ENST00000464244.1, and transcript_ENST00000608052.1.

In some embodiments, the MCAT pre-mRNA transcript is encoded by a genetic sequence with at least about 80%, 85%, 90%, 95%, 96%, 97%, 98%, 99% or 100% sequence identity to the Ensembl reference number ENSG00000100294.13 or a complement thereof. In some embodiments, the MCAT pre-mRNA transcript comprises a sequence with at least about 80%, 85%, 90%, 95%, 96%, 97%, 98%, 99% or 100% sequence identity to a MCAT pre-mRNA transcript or a complement thereof described herein.

In some embodiments, the targeted portion of the MCAT pre-mRNA comprises a sequence with at least 80%, 85%, 90%, 95%, 97%, or 100% sequence identity to a region comprising at least 8 contiguous nucleic acids of a sequence of premrna_ENST00000290429.11, premrna_ENST00000327555.5, premrna_ENST00000464244.1, premrna_ENST00000608052.1 or a sequence of Table 3 or complements thereof. In some embodiments, the targeted portion of the MCAT pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 676-SEQ ID NO: 683 or complements thereof. In some embodiments, the targeted portion of the MCAT pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a MCEE genomic sequence. In some embodiments, the ASO targets a NSAE pre-mRNA transcript from a MCEE genomic sequence comprising a NSAE exon. In some embodiments, the ASO targets a NSAE pre-mRNA transcript from a MCEE genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000244217.6, premrna_ENST00000413592.5, premrna_ENST00000462609.2, premrna_ENST00000486135.1, and premrna_ENST00000494660.6. In some embodiments, the ASO targets a MCEE pre-mRNA sequence comprising a NSAE exon. In some embodiments, the ASO targets a MCEE pre-mRNA sequence comprising an intron flanking the 3′ splice site of the NSAE exon. In some embodiments, the ASO targets a MCEE pre-mRNA sequence comprising an intron flanking the 5′ splice site of the NSAE exon. In some embodiments, the transcript is selected from the group consisting of transcript_ENST00000244217.6, transcript_ENST00000413592.5, transcript_ENST00000462609.2, transcript_ENST00000486135.1, and transcript_ENST00000494660.6.

In some embodiments, the MCEE pre-mRNA transcript is encoded by a genetic sequence with at least about 80%, 85%, 90%, 95%, 96%, 97%, 98%, 99% or 100% sequence identity to the Ensembl reference number ENSG00000124370.11 or a complement thereof. In some embodiments, the MCEE pre-mRNA transcript comprises a sequence with at least about 80%, 85%, 90%, 95%, 96%, 97%, 98%, 99% or 100% sequence identity to a MCEE pre-mRNA transcript or a complement thereof described herein.

In some embodiments, the targeted portion of the MCEE pre-mRNA comprises a sequence with at least 80%, 85%, 90%, 95%, 97%, or 100% sequence identity to a region comprising at least 8 contiguous nucleic acids of a sequence of premrna_ENST00000244217.6, premrna_ENST00000413592.5, premrna_ENST00000462609.2, premrna_ENST00000486135.1, premrna_ENST00000494660.6 or a sequence of Table 3 or complements thereof. In some embodiments, the targeted portion of the MCEE pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 684-SEQ ID NO: 689 or complements thereof. In some embodiments, the targeted portion of the MCEE pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a MPI genomic sequence. In some embodiments, the ASO targets a NSAE pre-mRNA transcript from a MPI genomic sequence comprising a NSAE exon. In some embodiments, the ASO targets a NSAE pre-mRNA transcript from a MPI genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000323744.10, premrna_ENST00000352410.9, premrna_ENST00000535694.5, premrna_ENST00000561470.5, premrna_ENST00000562606.5, premrna_ENST00000562800.5, premrna_ENST00000563422.5, premrna_ENST00000563786.5, premrna_ENST00000564003.5, premrna_ENST00000564633.5, premrna_ENST00000564692.1, premrna_ENST00000565576.5, premrna_ENST00000566377.5, premrna_ENST00000566556.1, premrna_ENST00000567116.5, premrna_ENST00000567132.5, premrna_ENST00000567177.1, premrna_ENST00000567570.5, premrna_ENST00000568303.1, premrna_ENST00000568828.5, premrna_ENST00000568840.1, premrna_ENST00000568907.5, premrna_ENST00000569233.5, and premrna_ENST00000569931.5. In some embodiments, the ASO targets a MPI pre-mRNA sequence comprising a NSAE exon. In some embodiments, the ASO targets a MPI pre-mRNA sequence comprising an intron flanking the 3′ splice site of the NSAE exon. In some embodiments, the ASO targets a MPI pre-mRNA sequence comprising an intron flanking the 5′ splice site of the NSAE exon. In some embodiments, the transcript is selected from the group consisting of transcript_ENST00000323744.10, transcript_ENST00000352410.9, transcript_ENST00000535694.5, transcript_ENST00000561470.5, transcript_ENST00000562606.5, transcript_ENST00000562800.5, transcript_ENST00000563422.5, transcript_ENST00000563786.5, transcript_ENST00000564003.5, transcript_ENST00000564633.5, transcript_ENST00000564692.1, transcript_ENST00000565576.5, transcript_ENST00000566377.5, transcript_ENST00000566556.1, transcript_ENST00000567116.5, transcript_ENST00000567132.5, transcript_ENST00000567177.1, transcript_ENST00000567570.5, transcript_ENST00000568303.1, transcript_ENST00000568828.5, transcript_ENST00000568840.1, transcript_ENST00000568907.5, transcript_ENST00000569233.5, and transcript_ENST00000569931.5.

In some embodiments, the MPI pre-mRNA transcript is encoded by a genetic sequence with at least about 80%, 85%, 90%, 95%, 96%, 97%, 98%, 99% or 100% sequence identity to the Ensembl reference number ENSG00000178802.18 or a complement thereof. In some embodiments, the MPI pre-mRNA transcript comprises a sequence with at least about 80%, 85%, 90%, 95%, 96%, 97%, 98%, 99% or 100% sequence identity to a MPI pre-mRNA transcript or a complement thereof described herein.

In some embodiments, the targeted portion of the MPI pre-mRNA comprises a sequence with at least 80%, 85%, 90%, 95%, 97%, or 100% sequence identity to a region comprising at least 8 contiguous nucleic acids of a sequence of premrna_ENST00000323744.10, premrna_ENST00000352410.9, premrna_ENST00000535694.5, premrna_ENST00000561470.5, premrna_ENST00000562606.5, premrna_ENST00000562800.5, premrna_ENST00000563422.5, premrna_ENST00000563786.5, premrna_ENST00000564003.5, premrna_ENST00000564633.5, premrna_ENST00000564692.1, premrna_ENST00000565576.5, premrna_ENST00000566377.5, premrna_ENST00000566556.1, premrna_ENST00000567116.5, premrna_ENST00000567132.5, premrna_ENST00000567177.1, premrna_ENST00000567570.5, premrna_ENST00000568303.1, premrna_ENST00000568828.5, premrna_ENST00000568840.1, premrna_ENST00000568907.5, premrna_ENST00000569233.5, premrna_ENST00000569931.5 or a sequence of Table 3 or complements thereof. In some embodiments, the targeted portion of the MPI pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 690-SEQ ID NO: 703 or complements thereof. In some embodiments, the targeted portion of the MPI pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a MSTO1 genomic sequence. In some embodiments, the ASO targets a NSAE pre-mRNA transcript from a MSTO1 genomic sequence comprising a NSAE exon. In some embodiments, the ASO targets a NSAE pre-mRNA transcript from a MSTO1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000245564.7, premrna_ENST00000368341.8, premrna_ENST00000460199.5, premrna_ENST00000462250.2, premrna_ENST00000465137.5, premrna_ENST00000466815.1, premrna_ENST00000471209.1, premrna_ENST00000473327.5, premrna_ENST00000475253.5, premrna_ENST00000478756.1, premrna_ENST00000482284.5, premrna_ENST00000483734.5, premrna_ENST00000483832.5, premrna_ENST00000488901.5, premrna_ENST00000490642.5, premrna_ENST00000490743.5, premrna_ENST00000491308.5, premrna_ENST00000494995.5, and premrna_ENST00000649846.1. In some embodiments, the ASO targets a MSTO1 pre-mRNA sequence comprising a NSAE exon. In some embodiments, the ASO targets a MSTO1 pre-mRNA sequence comprising an intron flanking the 3′ splice site of the NSAE exon. In some embodiments, the ASO targets a MSTO1 pre-mRNA sequence comprising an intron flanking the 5′ splice site of the NSAE exon. In some embodiments, the transcript is selected from the group consisting of transcript_ENST00000245564.7, transcript_ENST00000368341.8, transcript_ENST00000460199.5, transcript_ENST00000462250.2, transcript_ENST00000465137.5, transcript_ENST00000466815.1, transcript_ENST00000471209.1, transcript_ENST00000473327.5, transcript_ENST00000475253.5, transcript_ENST00000478756.1, transcript_ENST00000482284.5, transcript_ENST00000483734.5, transcript_ENST00000483832.5, transcript_ENST00000488901.5, transcript_ENST00000490642.5, transcript_ENST00000490743.5, transcript_ENST00000491308.5, transcript_ENST00000494995.5, and transcript_ENST00000649846.1.

In some embodiments, the MSTO1 pre-mRNA transcript is encoded by a genetic sequence with at least about 80%, 85%, 90%, 95%, 96%, 97%, 98%, 99% or 100% sequence identity to the Ensembl reference number ENSG00000125459.17 or a complement thereof. In some embodiments, the MSTO1 pre-mRNA transcript comprises a sequence with at least about 80%, 85%, 90%, 95%, 96%, 97%, 98%, 99% or 100% sequence identity to a MSTO1 pre-mRNA transcript or a complement thereof described herein.

In some embodiments, the targeted portion of the MSTO1 pre-mRNA comprises a sequence with at least 80%, 85%, 90%, 95%, 97%, or 100% sequence identity to a region comprising at least 8 contiguous nucleic acids of a sequence of premrna_ENST00000245564.7, premrna_ENST00000368341.8, premrna_ENST00000460199.5, premrna_ENST00000462250.2, premrna_ENST00000465137.5, premrna_ENST00000466815.1, premrna_ENST00000471209.1, premrna_ENST00000473327.5, premrna_ENST00000475253.5, premrna_ENST00000478756.1, premrna_ENST00000482284.5, premrna_ENST00000483734.5, premrna_ENST00000483832.5, premrna_ENST00000488901.5, premrna_ENST00000490642.5, premrna_ENST00000490743.5, premrna_ENST00000491308.5, premrna_ENST00000494995.5, premrna_ENST00000649846.1 or a sequence of Table 3 or complements thereof. In some embodiments, the targeted portion of the MSTO1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 704-SEQ ID NO: 707 or complements thereof. In some embodiments, the targeted portion of the MSTO1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NBEAL2 genomic sequence. In some embodiments, the ASO targets a NSAE pre-mRNA transcript from a NBEAL2 genomic sequence comprising a NSAE exon. In some embodiments, the ASO targets a NSAE pre-mRNA transcript from a NBEAL2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000416683.5, premrna_ENST00000423436.1, premrna_ENST00000441027.5, premrna_ENST00000443829.5, premrna_ENST00000450053.8, premrna_ENST00000461036.1, premrna_ENST00000469349.1, premrna_ENST00000475689.1, premrna_ENST00000476095.5, premrna_ENST00000477412.1, premrna_ENST00000486870.1, premrna_ENST00000651350.1, premrna_ENST00000651450.1, premrna_ENST00000651453.1, premrna_ENST00000651747.1, premrna_ENST00000652242.1, and premrna_ENST00000652744.1.

In some embodiments, the ASO targets a NBEAL2 pre-mRNA sequence comprising a NSAE exon. In some embodiments, the ASO targets a NBEAL2 pre-mRNA sequence comprising an intron flanking the 3′ splice site of the NSAE exon. In some embodiments, the ASO targets a NBEAL2 pre-mRNA sequence comprising an intron flanking the 5′ splice site of the NSAE exon. In some embodiments, the transcript is selected from the group consisting of transcript_ENST00000416683.5, transcript_ENST00000423436.1, transcript_ENST00000441027.5, transcript_ENST00000443829.5, transcript_ENST00000450053.8, transcript_ENST00000461036.1, transcript_ENST00000469349.1, transcript_ENST00000475689.1, transcript_ENST00000476095.5, transcript_ENST00000477412.1, transcript_ENST00000486870.1, transcript_ENST00000651350.1, transcript_ENST00000651450.1, transcript_ENST00000651453.1, transcript_ENST00000651747.1, transcript_ENST00000652242.1, and transcript_ENST00000652744.1.

In some embodiments, the NBEAL2 pre-mRNA transcript is encoded by a genetic sequence with at least about 80%, 85%, 90%, 95%, 96%, 97%, 98%, 99% or 100% sequence identity to the Ensembl reference number ENSG00000160796.18 or a complement thereof. In some embodiments, the NBEAL2 pre-mRNA transcript comprises a sequence with at least about 80%, 85%, 90%, 95%, 96%, 97%, 98%, 99% or 100% sequence identity to a NBEAL2 pre-mRNA transcript or a complement thereof described herein.

In some embodiments, the targeted portion of the NBEAL2 pre-mRNA comprises a sequence with at least 80%, 85%, 90%, 95%, 97%, or 100% sequence identity to a region comprising at least 8 contiguous nucleic acids of a sequence of premrna_ENST00000416683.5, premrna_ENST00000423436.1, premrna_ENST00000441027.5, premrna_ENST00000443829.5, premrna_ENST00000450053.8, premrna_ENST00000461036.1, premrna_ENST00000469349.1, premrna_ENST00000475689.1, premrna_ENST00000476095.5, premrna_ENST00000477412.1, premrna_ENST00000486870.1, premrna_ENST00000651350.1, premrna_ENST00000651450.1, premrna_ENST00000651453.1, premrna_ENST00000651747.1, premrna_ENST00000652242.1, premrna_ENST00000652744.1, or a sequence of Table 3 or complements thereof. In some embodiments, the targeted portion of the NBEAL2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 708-SEQ ID NO: 711 or complements thereof. In some embodiments, the targeted portion of the NBEAL2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NLE1 genomic sequence. In some embodiments, the ASO targets a NSAE pre-mRNA transcript from a NLE1 genomic sequence comprising a NSAE exon. In some embodiments, the ASO targets a NSAE pre-mRNA transcript from a NLE1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000360831.9, premrna_ENST00000442241.9, premrna_ENST00000586869.5, premrna_ENST00000588019.1, premrna_ENST00000588642.1, premrna_ENST00000589367.5, and premrna_ENST00000593176.1. In some embodiments, the ASO targets a NLE1 pre-mRNA sequence comprising a NSAE exon. In some embodiments, the ASO targets a NLE1 pre-mRNA sequence comprising an intron flanking the 3′ splice site of the NSAE exon. In some embodiments, the ASO targets a NLE1 pre-mRNA sequence comprising an intron flanking the 5′ splice site of the NSAE exon. In some embodiments, the transcript is selected from the group consisting of transcript_ENST00000360831.9, transcript_ENST00000442241.9, transcript_ENST00000586869.5, transcript_ENST00000588019.1, transcript_ENST00000588642.1, transcript_ENST00000589367.5, and transcript_ENST00000593176.1.

In some embodiments, the NLE1 pre-mRNA transcript is encoded by a genetic sequence with at least about 80%, 85%, 90%, 95%, 96%, 97%, 98%, 99% or 100% sequence identity to the Ensembl reference number ENSG00000073536.18 or a complement thereof. In some embodiments, the NLE1 pre-mRNA transcript comprises a sequence with at least about 80%, 85%, 90%, 95%, 96%, 97%, 98%, 99% or 100% sequence identity to a NLE1 pre-mRNA transcript or a complement thereof described herein.

In some embodiments, the targeted portion of the NLE1 pre-mRNA comprises a sequence with at least 80%, 85%, 90%, 95%, 97%, or 100% sequence identity to a region comprising at least 8 contiguous nucleic acids of a sequence of premrna_ENST00000360831.9, premrna_ENST00000442241.9, premrna_ENST00000586869.5, premrna_ENST00000588019.1, premrna_ENST00000588642.1, premrna_ENST00000589367.5, premrna_ENST00000593176.1, or a sequence of Table 3 or complements thereof. In some embodiments, the targeted portion of the NLE1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 712-SEQ ID NO: 715 or complements thereof. In some embodiments, the targeted portion of the NLE1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NLRC5 genomic sequence. In some embodiments, the ASO targets a NSAE pre-mRNA transcript from a NLRC5 genomic sequence comprising a NSAE exon. In some embodiments, the ASO targets a NSAE pre-mRNA transcript from a NLRC5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000262510.10, premrna_ENST00000399221.3, premrna_ENST00000436936.5, premrna_ENST00000534927.5, premrna_ENST00000534931.1, premrna_ENST00000535284.1, premrna_ENST00000535658.1, premrna_ENST00000536231.1, premrna_ENST00000537056.5, premrna_ENST00000538059.5, premrna_ENST00000538110.5, premrna_ENST00000538273.5, premrna_ENST00000538453.5, premrna_ENST00000538778.5, premrna_ENST00000538805.5, premrna_ENST00000538930.5, premrna_ENST00000539144.5, premrna_ENST00000539881.5, premrna_ENST00000540182.5, premrna_ENST00000541020.1, premrna_ENST00000543030.5, premrna_ENST00000543049.1, premrna_ENST00000543103.1, premrna_ENST00000543141.5, premrna_ENST00000543402.1, premrna_ENST00000544189.1, premrna_ENST00000544641.5, premrna_ENST00000545081.5, and premrna_ENST00000545349.1. In some embodiments, the ASO targets a NLRC5 pre-mRNA sequence comprising a NSAE exon. In some embodiments, the ASO targets a NLRC5 pre-mRNA sequence comprising an intron flanking the 3′ splice site of the NSAE exon. In some embodiments, the ASO targets a NLRC5 pre-mRNA sequence comprising an intron flanking the 5′ splice site of the NSAE exon. In some embodiments, the transcript is selected from the group consisting of transcript_ENST00000262510.10, transcript_ENST00000399221.3, transcript_ENST00000436936.5, transcript_ENST00000534927.5, transcript_ENST00000534931.1, transcript_ENST00000535284.1, transcript_ENST00000535658.1, transcript_ENST00000536231.1, transcript_ENST00000537056.5, transcript_ENST00000538059.5, transcript_ENST00000538110.5, transcript_ENST00000538273.5, transcript_ENST00000538453.5, transcript_ENST00000538778.5, transcript_ENST00000538805.5, transcript_ENST00000538930.5, transcript_ENST00000539144.5, transcript_ENST00000539881.5, transcript_ENST00000540182.5, transcript_ENST00000541020.1, transcript_ENST00000543030.5, transcript_ENST00000543049.1, transcript_ENST00000543103.1, transcript_ENST00000543141.5, transcript_ENST00000543402.1, transcript_ENST00000544189.1, transcript_ENST00000544641.5, transcript_ENST00000545081.5, transcript_ENST00000545349.1.

In some embodiments, the NLRC5 pre-mRNA transcript is encoded by a genetic sequence with at least about 80%, 85%, 90%, 95%, 96%, 97%, 98%, 99% or 100% sequence identity to the Ensembl reference number ENSG00000140853.15 or a complement thereof. In some embodiments, the NLRC5 pre-mRNA transcript comprises a sequence with at least about 80%, 85%, 90%, 95%, 96%, 97%, 98%, 99% or 100% sequence identity to a NLRC5 pre-mRNA transcript or a complement thereof described herein.

In some embodiments, the targeted portion of the NLRC5 pre-mRNA comprises a sequence with at least 80%, 85%, 90%, 95%, 97%, or 100% sequence identity to a region comprising at least 8 contiguous nucleic acids of a sequence of premrna_ENST00000262510.10, premrna_ENST00000399221.3, premrna_ENST00000436936.5, premrna_ENST00000534927.5, premrna_ENST00000534931.1, premrna_ENST00000535284.1, premrna_ENST00000535658.1, premrna_ENST00000536231.1, premrna_ENST00000537056.5, premrna_ENST00000538059.5, premrna_ENST00000538110.5, premrna_ENST00000538273.5, premrna_ENST00000538453.5, premrna_ENST00000538778.5, premrna_ENST00000538805.5, premrna_ENST00000538930.5, premrna_ENST00000539144.5, premrna_ENST00000539881.5, premrna_ENST00000540182.5, premrna_ENST00000541020.1, premrna_ENST00000543030.5, premrna_ENST00000543049.1, premrna_ENST00000543103.1, premrna_ENST00000543141.5, premrna_ENST00000543402.1, premrna_ENST00000544189.1, premrna_ENST00000544641.5, premrna_ENST00000545081.5, premrna_ENST00000545349.1, or a sequence of Table 3 or complements thereof. In some embodiments, the targeted portion of the NLRC5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 716-SEQ ID NO: 719 or complements thereof. In some embodiments, the targeted portion of the NLRC5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NOM1 genomic sequence. In some embodiments, the ASO targets a NSAE pre-mRNA transcript from a NOM1 genomic sequence comprising a NSAE exon. In some embodiments, the ASO targets a NSAE pre-mRNA transcript from a NOM1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000275820.4, premrna_ENST00000460332.1, premrna_ENST00000469271.1, premrna_ENST00000472491.1, premrna_ENST00000475176.1, premrna_ENST00000485661.1, premrna_ENST00000486131.1, and premrna_ENST00000489850.5. In some embodiments, the ASO targets a NOM1 pre-mRNA sequence comprising a NSAE exon. In some embodiments, the ASO targets a NOM1 pre-mRNA sequence comprising an intron flanking the 3′ splice site of the NSAE exon. In some embodiments, the ASO targets a NOM1 pre-mRNA sequence comprising an intron flanking the 5′ splice site of the NSAE exon. In some embodiments, the transcript is selected from the group consisting of transcript_ENST00000275820.4, transcript_ENST00000460332.1, transcript_ENST00000469271.1, transcript_ENST00000472491.1, transcript_ENST00000475176.1, transcript_ENST00000485661.1, transcript_ENST00000486131.1, and transcript_ENST00000489850.5.

In some embodiments, the NOM1 pre-mRNA transcript is encoded by a genetic sequence with at least about 80%, 85%, 90%, 95%, 96%, 97%, 98%, 99% or 100% sequence identity to the Ensembl reference number ENSG00000146909.8 or a complement thereof. In some embodiments, the NOM1 pre-mRNA transcript comprises a sequence with at least about 80%, 85%, 90%, 95%, 96%, 97%, 98%, 99% or 100% sequence identity to a NOM1 pre-mRNA transcript or a complement thereof described herein.

In some embodiments, the targeted portion of the NOM1 pre-mRNA comprises a sequence with at least 80%, 85%, 90%, 95%, 97%, or 100% sequence identity to a region comprising at least 8 contiguous nucleic acids of a sequence of premrna_ENST00000275820.4, premrna_ENST00000460332.1, premrna_ENST00000469271.1, premrna_ENST00000472491.1, premrna_ENST00000475176.1, premrna_ENST00000485661.1, premrna_ENST00000486131.1, premrna_ENST00000489850.5 or a sequence of Table 3 or complements thereof. In some embodiments, the targeted portion of the NOM1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 720-SEQ ID NO: 723 or complements thereof. In some embodiments, the targeted portion of the NOM1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NOP58 genomic sequence. In some embodiments, the ASO targets a NSAE pre-mRNA transcript from a NOP58 genomic sequence comprising a NSAE exon. In some embodiments, the ASO targets a NSAE pre-mRNA transcript from a NOP58 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000264279.10, premrna_ENST00000426814.5, premrna_ENST00000433543.2, premrna_ENST00000467734.5, premrna_ENST00000472050.5, premrna_ENST00000478508.1, premrna_ENST00000478941.1, premrna_ENST00000488403.5, premrna_ENST00000492688.5, and premrna_ENST00000492740.5. In some embodiments, the ASO targets a NOP58 pre-mRNA sequence comprising a NSAE exon. In some embodiments, the ASO targets a NOP58 pre-mRNA sequence comprising an intron flanking the 3′ splice site of the NSAE exon. In some embodiments, the ASO targets a NOP58 pre-mRNA sequence comprising an intron flanking the 5′ splice site of the NSAE exon. In some embodiments, the transcript is selected from the group consisting of transcript_ENST00000264279.10, transcript_ENST00000426814.5, transcript_ENST00000433543.2, transcript_ENST00000467734.5, transcript_ENST00000472050.5, transcript_ENST00000478508.1, transcript_ENST00000478941.1, transcript_ENST00000488403.5, transcript_ENST00000492688.5, and transcript_ENST00000492740.5.

In some embodiments, the NOP58 pre-mRNA transcript is encoded by a genetic sequence with at least about 80%, 85%, 90%, 95%, 96%, 97%, 98%, 99% or 100% sequence identity to the Ensembl reference number ENSG00000055044.11 or a complement thereof. In some embodiments, the NOP58 pre-mRNA transcript comprises a sequence with at least about 80%, 85%, 90%, 95%, 96%, 97%, 98%, 99% or 100% sequence identity to a NOP58 pre-mRNA transcript or a complement thereof described herein.

In some embodiments, the targeted portion of the NOP58 pre-mRNA comprises a sequence with at least 80%, 85%, 90%, 95%, 97%, or 100% sequence identity to a region comprising at least 8 contiguous nucleic acids of a sequence of premrna_ENST00000264279.10, premrna_ENST00000426814.5, premrna_ENST00000433543.2, premrna_ENST00000467734.5, premrna_ENST00000472050.5, premrna_ENST00000478508.1, premrna_ENST00000478941.1, premrna_ENST00000488403.5, premrna_ENST00000492688.5, premrna_ENST00000492740.5 or a sequence of Table 3 or complements thereof. In some embodiments, the targeted portion of the NOP58 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 724-SEQ ID NO: 727 or complements thereof. In some embodiments, the targeted portion of the NOP58 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NPHP1 genomic sequence. In some embodiments, the ASO targets a NSAE pre-mRNA transcript from a NPHP1 genomic sequence comprising a NSAE exon. In some embodiments, the ASO targets a NSAE pre-mRNA transcript from a NPHP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000316534.8, premrna_ENST00000355301.8, premrna_ENST00000393272.7, premrna_ENST00000417665.5, premrna_ENST00000418527.1, premrna_ENST00000422492.1, premrna_ENST00000445609.6, premrna_ENST00000449600.1, premrna_ENST00000461707.5, premrna_ENST00000493051.1, and premrna_ENST00000496524.5. In some embodiments, the ASO targets a NPHP1 pre-mRNA sequence comprising a NSAE exon. In some embodiments, the ASO targets a NPHP1 pre-mRNA sequence comprising an intron flanking the 3′ splice site of the NSAE exon. In some embodiments, the ASO targets a NPHP1 pre-mRNA sequence comprising an intron flanking the 5′ splice site of the NSAE exon. In some embodiments, the transcript is selected from the group consisting of transcript_ENST00000316534.8, transcript_ENST00000355301.8, transcript_ENST00000393272.7, transcript_ENST00000417665.5, transcript_ENST00000418527.1, transcript_ENST00000422492.1, transcript_ENST00000445609.6, transcript_ENST00000449600.1, transcript_ENST00000461707.5, transcript_ENST00000493051.1, and transcript_ENST00000496524.5.

In some embodiments, the NPHP1 pre-mRNA transcript is encoded by a genetic sequence with at least about 80%, 85%, 90%, 95%, 96%, 97%, 98%, 99% or 100% sequence identity to the Ensembl reference number ENSG00000144061.13 or a complement thereof. In some embodiments, the NPHP1 pre-mRNA transcript comprises a sequence with at least about 80%, 85%, 90%, 95%, 96%, 97%, 98%, 99% or 100% sequence identity to a NPHP1 pre-mRNA transcript or a complement thereof described herein.

In some embodiments, the targeted portion of the NPHP1 pre-mRNA comprises a sequence with at least 80%, 85%, 90%, 95%, 97%, or 100% sequence identity to a region comprising at least 8 contiguous nucleic acids of a sequence of premrna_ENST00000316534.8, premrna_ENST00000355301.8, premrna_ENST00000393272.7, premrna_ENST00000417665.5, premrna_ENST00000418527.1, premrna_ENST00000422492.1, premrna_ENST00000445609.6, premrna_ENST00000449600.1, premrna_ENST00000461707.5, premrna_ENST00000493051.1, premrna_ENST00000496524.5 or a sequence of Table 3 or complements thereof. In some embodiments, the targeted portion of the NPHP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 728-SEQ ID NO: 733 or complements thereof. In some embodiments, the targeted portion of the NPHP1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NPR1 genomic sequence. In some embodiments, the ASO targets a NSAE pre-mRNA transcript from a NPR1 genomic sequence comprising a NSAE exon. In some embodiments, the ASO targets a NSAE pre-mRNA transcript from a NPR1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000368677.2, premrna_ENST00000368680.4, and premrna_ENST00000413826.1. In some embodiments, the ASO targets a NPR1 pre-mRNA sequence comprising a NSAE exon. In some embodiments, the ASO targets a NPR1 pre-mRNA sequence comprising an intron flanking the 3′ splice site of the NSAE exon. In some embodiments, the ASO targets a NPR1 pre-mRNA sequence comprising an intron flanking the 5′ splice site of the NSAE exon. In some embodiments, the transcript is selected from the group consisting of transcript_ENST00000368677.2, transcript_ENST00000368680.4, and transcript_ENST00000413826.1.

In some embodiments, the NPR1 pre-mRNA transcript is encoded by a genetic sequence with at least about 80%, 85%, 90%, 95%, 96%, 97%, 98%, 99% or 100% sequence identity to the Ensembl reference number ENSG00000169418.10 or a complement thereof. In some embodiments, the NPR1 pre-mRNA transcript comprises a sequence with at least about 80%, 85%, 90%, 95%, 96%, 97%, 98%, 99% or 100% sequence identity to a NPR1 pre-mRNA transcript or a complement thereof described herein.

In some embodiments, the targeted portion of the NPR1 pre-mRNA comprises a sequence with at least 80%, 85%, 90%, 95%, 97%, or 100% sequence identity to a region comprising at least 8 contiguous nucleic acids of a sequence of premrna_ENST00000368677.2, premrna_ENST00000368680.4, premrna_ENST00000413826.1 or a sequence of Table 3 or complements thereof. In some embodiments, the targeted portion of the NPR1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 734-SEQ ID NO: 737 or complements thereof. In some embodiments, the targeted portion of the NPR1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a NUP188 genomic sequence. In some embodiments, the ASO targets a NSAE pre-mRNA transcript from a NUP188 genomic sequence comprising a NSAE exon. In some embodiments, the ASO targets a NSAE pre-mRNA transcript from a NUP188 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000372577.2, premrna_ENST00000464729.1, premrna_ENST00000465344.1, premrna_ENST00000467044.1, premrna_ENST00000477069.5, premrna_ENST00000485158.1, premrna_ENST00000487952.1, premrna_ENST00000491502.1, premrna_ENST00000491990.5, premrna_ENST00000495726.1, and premrna_ENST00000550219.1. In some embodiments, the ASO targets a NUP188 pre-mRNA sequence comprising a NSAE exon. In some embodiments, the ASO targets a NUP188 pre-mRNA sequence comprising an intron flanking the 3′ splice site of the NSAE exon. In some embodiments, the ASO targets a NUP188 pre-mRNA sequence comprising an intron flanking the 5′ splice site of the NSAE exon. In some embodiments, the transcript is selected from the group consisting of transcript_ENST00000372577.2, transcript_ENST00000464729.1, transcript_ENST00000465344.1, transcript_ENST00000467044.1, transcript_ENST00000477069.5, transcript_ENST00000485158.1, transcript_ENST00000487952.1, transcript_ENST00000491502.1, transcript_ENST00000491990.5, transcript_ENST00000495726.1, and transcript_ENST00000550219.1.

In some embodiments, the NUP188 pre-mRNA transcript is encoded by a genetic sequence with at least about 80%, 85%, 90%, 95%, 96%, 97%, 98%, 99% or 100% sequence identity to the Ensembl reference number ENSG00000095319.14 or a complement thereof. In some embodiments, the NUP188 pre-mRNA transcript comprises a sequence with at least about 80%, 85%, 90%, 95%, 96%, 97%, 98%, 99% or 100% sequence identity to a NUP188 pre-mRNA transcript or a complement thereof described herein.

In some embodiments, the targeted portion of the NUP188 pre-mRNA comprises a sequence with at least 80%, 85%, 90%, 95%, 97%, or 100% sequence identity to a region comprising at least 8 contiguous nucleic acids of a sequence of premrna_ENST00000372577.2, premrna_ENST00000464729.1, premrna_ENST00000465344.1, premrna_ENST00000467044.1, premrna_ENST00000477069.5, premrna_ENST00000485158.1, premrna_ENST00000487952.1, premrna_ENST00000491502.1, premrna_ENST00000491990.5, premrna_ENST00000495726.1, premrna_ENST00000550219.1 or a sequence of Table 3 or complements thereof. In some embodiments, the targeted portion of the NUP188 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 738-SEQ ID NO: 741 or complements thereof. In some embodiments, the targeted portion of the NUP188 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a OSGEP genomic sequence. In some embodiments, the ASO targets a NSAE pre-mRNA transcript from a OSGEP genomic sequence comprising a NSAE exon. In some embodiments, the ASO targets a NSAE pre-mRNA transcript from a OSGEP genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000206542.9, premrna_ENST00000553292.1, premrna_ENST00000553640.3, premrna_ENST00000554249.5, premrna_ENST00000554699.1, premrna_ENST00000554915.1, premrna_ENST00000555223.5, premrna_ENST00000555656.5, premrna_ENST00000555785.2, premrna_ENST00000556124.3, premrna_ENST00000556252.1, and premrna_ENST00000556439.1. In some embodiments, the ASO targets a OSGEP pre-mRNA sequence comprising a NSAE exon. In some embodiments, the ASO targets a OSGEP pre-mRNA sequence comprising an intron flanking the 3′ splice site of the NSAE exon. In some embodiments, the ASO targets a OSGEP pre-mRNA sequence comprising an intron flanking the 5′ splice site of the NSAE exon. In some embodiments, the transcript is selected from the group consisting of transcript_ENST00000206542.9, transcript_ENST00000553292.1, transcript_ENST00000553640.3, transcript_ENST00000554249.5, transcript_ENST00000554699.1, transcript_ENST00000554915.1, transcript_ENST00000555223.5, transcript_ENST00000555656.5, transcript_ENST00000555785.2, transcript_ENST00000556124.3, transcript_ENST00000556252.1, and transcript_ENST00000556439.1.

In some embodiments, the OSGEP pre-mRNA transcript is encoded by a genetic sequence with at least about 80%, 85%, 90%, 95%, 96%, 97%, 98%, 99% or 100% sequence identity to the Ensembl reference number ENSG00000092094.11 or a complement thereof. In some embodiments, the OSGEP pre-mRNA transcript comprises a sequence with at least about 80%, 85%, 90%, 95%, 96%, 97%, 98%, 99% or 100% sequence identity to a OSGEP pre-mRNA transcript or a complement thereof described herein.

In some embodiments, the targeted portion of the OSGEP pre-mRNA comprises a sequence with at least 80%, 85%, 90%, 95%, 97%, or 100% sequence identity to a region comprising at least 8 contiguous nucleic acids of a sequence of premrna_ENST00000206542.9, premrna_ENST00000553292.1, premrna_ENST00000553640.3, premrna_ENST00000554249.5, premrna_ENST00000554699.1, premrna_ENST00000554915.1, premrna_ENST00000555223.5, premrna_ENST00000555656.5, premrna_ENST00000555785.2, premrna_ENST00000556124.3, premrna_ENST00000556252.1, premrna_ENST00000556439.1, or a sequence of Table 3 or complements thereof. In some embodiments, the targeted portion of the OSGEP pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 742-SEQ ID NO: 745 or complements thereof. In some embodiments, the targeted portion of the OSGEP pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PABPC4 genomic sequence. In some embodiments, the ASO targets a NSAE pre-mRNA transcript from a PABPC4 genomic sequence comprising a NSAE exon. In some embodiments, the ASO targets a NSAE pre-mRNA transcript from a PABPC4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000372856.7, premrna_ENST00000372857.7, premrna_ENST00000372858.8, premrna_ENST00000372862.7, premrna_ENST00000421687.6, premrna_ENST00000437136.5, premrna_ENST00000451091.2, premrna_ENST00000461578.1, premrna_ENST00000468476.1, premrna_ENST00000470443.5, premrna_ENST00000474378.1, premrna_ENST00000477556.1, premrna_ENST00000482028.5, premrna_ENST00000483770.1, premrna_ENST00000484555.1, premrna_ENST00000492468.5, premrna_ENST00000492519.1, premrna_ENST00000513632.2, premrna_ENST00000525045.5, premrna_ENST00000525669.5, premrna_ENST00000525751.5, premrna_ENST00000527718.1, premrna_ENST00000529216.1, premma_ENST00000530186.1, and premrna_ENST00000531243.2. In some embodiments, the ASO targets a PABPC4 pre-mRNA sequence comprising a NSAE exon. In some embodiments, the ASO targets a PABPC4 pre-mRNA sequence comprising an intron flanking the 3′ splice site of the NSAE exon. In some embodiments, the ASO targets a PABPC4 pre-mRNA sequence comprising an intron flanking the 5′ splice site of the NSAE exon. In some embodiments, the transcript is selected from the group consisting of transcript_ENST00000372856.7, transcript_ENST00000372857.7, transcript_ENST00000372858.8, transcript_ENST00000372862.7, transcript_ENST00000421687.6, transcript_ENST00000437136.5, transcript_ENST00000451091.2, transcript_ENST00000461578.1, transcript_ENST00000468476.1, transcript_ENST00000470443.5, transcript_ENST00000474378.1, transcript_ENST00000477556.1, transcript_ENST00000482028.5, transcript_ENST00000483770.1, transcript_ENST00000484555.1, transcript_ENST00000492468.5, transcript_ENST00000492519.1, transcript_ENST00000513632.2, transcript_ENST00000525045.5, transcript_ENST00000525669.5, transcript_ENST00000525751.5, transcript_ENST00000527718.1, transcript_ENST00000529216.1, transcript_ENST00000530186.1, and transcript_ENST00000531243.2.

In some embodiments, the PABPC4 pre-mRNA transcript is encoded by a genetic sequence with at least about 80%, 85%, 90%, 95%, 96%, 97%, 98%, 99% or 100% sequence identity to the Ensembl reference number ENSG00000090621.14 or a complement thereof. In some embodiments, the PABPC4 pre-mRNA transcript comprises a sequence with at least about 80%, 85%, 90%, 95%, 96%, 97%, 98%, 99% or 100% sequence identity to a PABPC4 pre-mRNA transcript or a complement thereof described herein.

In some embodiments, the targeted portion of the PABPC4 pre-mRNA comprises a sequence with at least 80%, 85%, 90%, 95%, 97%, or 100% sequence identity to a region comprising at least 8 contiguous nucleic acids of a sequence of premrna_ENST00000372856.7, premrna_ENST00000372857.7, premrna_ENST00000372858.8, premrna_ENST00000372862.7, premrna_ENST00000421687.6, premrna_ENST00000437136.5, premrna_ENST00000451091.2, premrna_ENST00000461578.1, premrna_ENST00000468476.1, premrna_ENST00000470443.5, premrna_ENST00000474378.1, premrna_ENST00000477556.1, premrna_ENST00000482028.5, premrna_ENST00000483770.1, premrna_ENST00000484555.1, premrna_ENST00000492468.5, premrna_ENST00000492519.1, premrna_ENST00000513632.2, premrna_ENST00000525045.5, premrna_ENST00000525669.5, premrna_ENST00000525751.5, premrna_ENST00000527718.1, premrna_ENST00000529216.1, premrna_ENST00000530186.1, premrna_ENST00000531243.2 or a sequence of Table 3 or complements thereof. In some embodiments, the targeted portion of the PABPC4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 746-SEQ ID NO: 749 or complements thereof. In some embodiments, the targeted portion of the PABPC4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PCOLCE genomic sequence. In some embodiments, the ASO targets a NSAE pre-mRNA transcript from a PCOLCE genomic sequence comprising a NSAE exon. In some embodiments, the ASO targets a NSAE pre-mRNA transcript from a PCOLCE genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000223061.6, premrna_ENST00000460002.1, premrna_ENST00000462260.1, premrna_ENST00000468214.1, premrna_ENST00000472348.1, premrna_ENST00000482863.1, premrna_ENST00000486440.5, premrna_ENST00000487172.1, premrna_ENST00000490909.1, and premrna_ENST00000496269.1. In some embodiments, the ASO targets a PCOLCE pre-mRNA sequence comprising a NSAE exon. In some embodiments, the ASO targets a PCOLCE pre-mRNA sequence comprising an intron flanking the 3′ splice site of the NSAE exon. In some embodiments, the ASO targets a PCOLCE pre-mRNA sequence comprising an intron flanking the 5′ splice site of the NSAE exon. In some embodiments, the transcript is selected from the group consisting of transcript_ENST00000223061.6, transcript_ENST00000460002.1, transcript_ENST00000462260.1, transcript_ENST00000468214.1, transcript_ENST00000472348.1, transcript_ENST00000482863.1, transcript_ENST00000486440.5, transcript_ENST00000487172.1, transcript_ENST00000490909.1, and transcript_ENST00000496269.1.

In some embodiments, the PCOLCE pre-mRNA transcript is encoded by a genetic sequence with at least about 80%, 85%, 90%, 95%, 96%, 97%, 98%, 99% or 100% sequence identity to the Ensembl reference number ENSG00000106333.13 or ENSG00000224729.6 or a complement thereof. In some embodiments, the PCOLCE pre-mRNA transcript comprises a sequence with at least about 80%, 85%, 90%, 95%, 96%, 97%, 98%, 99% or 100% sequence identity to a PCOLCE pre-mRNA transcript or a complement thereof described herein.

In some embodiments, the targeted portion of the PCOLCE pre-mRNA comprises a sequence with at least 80%, 85%, 90%, 95%, 97%, or 100% sequence identity to a region comprising at least 8 contiguous nucleic acids of a sequence of premrna_ENST00000223061.6, premrna_ENST00000460002.1, premrna_ENST00000462260.1, premrna_ENST00000468214.1, premrna_ENST00000472348.1, premrna_ENST00000482863.1, premrna_ENST00000486440.5, premrna_ENST00000487172.1, premrna_ENST00000490909.1, premrna_ENST00000496269.1 or a sequence of Table 3 or complements thereof. In some embodiments, the targeted portion of the PCOLCE pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 750-SEQ ID NO: 753 or complements thereof. In some embodiments, the targeted portion of the PCOLCE pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PHKA2 genomic sequence. In some embodiments, the ASO targets a NSAE pre-mRNA transcript from a PHKA2 genomic sequence comprising a NSAE exon. In some embodiments, the ASO targets a NSAE pre-mRNA transcript from a PHKA2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000379942.5, premrna_ENST00000464455.1, premrna_ENST00000469485.5, premrna_ENST00000469645.5, premrna_ENST00000473597.1, premrna_ENST00000473739.5, premrna_ENST00000481718.1, and premrna_ENST00000486231.2. In some embodiments, the ASO targets a PHKA2 pre-mRNA sequence comprising a NSAE exon. In some embodiments, the ASO targets a PHKA2 pre-mRNA sequence comprising an intron flanking the 3′ splice site of the NSAE exon. In some embodiments, the ASO targets a PHKA2 pre-mRNA sequence comprising an intron flanking the 5′ splice site of the NSAE exon. In some embodiments, the transcript is selected from the group consisting of transcript_ENST00000379942.5, transcript_ENST00000464455.1, transcript_ENST00000469485.5, transcript_ENST00000469645.5, transcript_ENST00000473597.1, transcript_ENST00000473739.5, transcript_ENST00000481718.1, and transcript_ENST00000486231.2.

In some embodiments, the PHKA2 pre-mRNA transcript is encoded by a genetic sequence with at least about 80%, 85%, 90%, 95%, 96%, 97%, 98%, 99% or 100% sequence identity to the Ensembl reference number ENSG00000044446.12 or a complement thereof. In some embodiments, the PHKA2 pre-mRNA transcript comprises a sequence with at least about 80%, 85%, 90%, 95%, 96%, 97%, 98%, 99% or 100% sequence identity to a PHKA2 pre-mRNA transcript or a complement thereof described herein.

In some embodiments, the targeted portion of the PHKA2 pre-mRNA comprises a sequence with at least 80%, 85%, 90%, 95%, 97%, or 100% sequence identity to a region comprising at least 8 contiguous nucleic acids of a sequence of premrna_ENST00000379942.5, premrna_ENST00000464455.1, premrna_ENST00000469485.5, premrna_ENST00000469645.5, premrna_ENST00000473597.1, premrna_ENST00000473739.5, premrna_ENST00000481718.1, premrna_ENST00000486231.2 or a sequence of Table 3 or complements thereof. In some embodiments, the targeted portion of the PHKA2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 754-SEQ ID NO: 757 or complements thereof. In some embodiments, the targeted portion of the PHKA2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PIDD1 genomic sequence. In some embodiments, the ASO targets a NSAE pre-mRNA transcript from a PIDD1 genomic sequence comprising a NSAE exon. In some embodiments, the ASO targets a NSAE pre-mRNA transcript from a PIDD1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000347755.10, premrna_ENST00000411829.6, premrna_ENST00000524486.5, premrna_ENST00000525028.6, premrna_ENST00000527357.5, premrna_ENST00000527812.1, premrna_ENST00000528122.1, premrna_ENST00000530911.1, premrna_ENST00000531286.5, premrna_ENST00000534525.5, and premrna_ENST00000534649.1. In some embodiments, the ASO targets a PIDD1 pre-mRNA sequence comprising a NSAE exon. In some embodiments, the ASO targets a PIDD1 pre-mRNA sequence comprising an intron flanking the 3′ splice site of the NSAE exon. In some embodiments, the ASO targets a PIDD1 pre-mRNA sequence comprising an intron flanking the 5′ splice site of the NSAE exon. In some embodiments, the transcript is selected from the group consisting of transcript_ENST00000347755.10, transcript_ENST00000411829.6, transcript_ENST00000524486.5, transcript_ENST00000525028.6, transcript_ENST00000527357.5, transcript_ENST00000527812.1, transcript_ENST00000528122.1, transcript_ENST00000530911.1, transcript_ENST00000531286.5, transcript_ENST00000534525.5, and transcript_ENST00000534649.1.

In some embodiments, the PIDD1 pre-mRNA transcript is encoded by a genetic sequence with at least about 80%, 85%, 90%, 95%, 96%, 97%, 98%, 99% or 100% sequence identity to the Ensembl reference number ENSG00000177595.18 or a complement thereof. In some embodiments, the PIDD1 pre-mRNA transcript comprises a sequence with at least about 80%, 85%, 90%, 95%, 96%, 97%, 98%, 99% or 100% sequence identity to a PIDD1 pre-mRNA transcript or a complement thereof described herein.

In some embodiments, the targeted portion of the PIDD1 pre-mRNA comprises a sequence with at least 80%, 85%, 90%, 95%, 97%, or 100% sequence identity to a region comprising at least 8 contiguous nucleic acids of a sequence of premrna_ENST00000347755.10, premrna_ENST00000411829.6, premrna_ENST00000524486.5, premrna_ENST00000525028.6, premrna_ENST00000527357.5, premrna_ENST00000527812.1, premrna_ENST00000528122.1, premrna_ENST00000530911.1, premrna_ENST00000531286.5, premrna_ENST00000534525.5, premrna_ENST00000534649.1 or a sequence of Table 3 or complements thereof. In some embodiments, the targeted portion of the PIDD1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 758-SEQ ID NO: 765 or complements thereof. In some embodiments, the targeted portion of the PIDD1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PKD1 genomic sequence. In some embodiments, the ASO targets a NSAE pre-mRNA transcript from a PKD1 genomic sequence comprising a NSAE exon. In some embodiments, the ASO targets a NSAE pre-mRNA transcript from a PKD1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000262304.9, premrna_ENST00000415938.7, premrna_ENST00000423118.5, premrna_ENST00000468674.5, premrna_ENST00000469241.2, premrna_ENST00000469851.1, premrna_ENST00000471603.6, premrna_ENST00000472577.1, premrna_ENST00000472659.1, premrna_ENST00000473780.2, premrna_ENST00000474088.1, premrna_ENST00000475889.1, premrna_ENST00000480227.5, premrna_ENST00000483024.1, premrna_ENST00000483558.5, premrna_ENST00000483731.5, premrna_ENST00000483814.1, premrna_ENST00000485120.1, premrna_ENST00000486339.6, premrna_ENST00000487932.5, premrna_ENST00000488185.2, premrna_ENST00000496574.6, premrna_ENST00000561668.5, premrna_ENST00000561991.5, premrna_ENST00000562297.5, premrna_ENST00000562425.1, premrna_ENST00000564313.1, premrna_ENST00000564865.5, premrna_ENST00000564890.1, premrna_ENST00000565639.6, premrna_ENST00000566784.5, premrna_ENST00000566905.5, premrna_ENST00000567355.1, premrna_ENST00000567946.1, premrna_ENST00000568591.5, premrna_ENST00000568796.1, premrna_ENST00000569983.5, premrna_ENST00000570150.1, premrna_ENST00000570193.5, and premrna_ENST00000570253.5. In some embodiments, the ASO targets a PKD1 pre-mRNA sequence comprising a NSAE exon. In some embodiments, the ASO targets a PKD1 pre-mRNA sequence comprising an intron flanking the 3′ splice site of the NSAE exon. In some embodiments, the ASO targets a PKD1 pre-mRNA sequence comprising an intron flanking the 5′ splice site of the NSAE exon. In some embodiments, the transcript is selected from the group consisting of transcript_ENST00000262304.9, transcript_ENST00000415938.7, transcript_ENST00000423118.5, transcript_ENST00000468674.5, transcript_ENST00000469241.2, transcript_ENST00000469851.1, transcript_ENST00000471603.6, transcript_ENST00000472577.1, transcript_ENST00000472659.1, transcript_ENST00000473780.2, transcript_ENST00000474088.1, transcript_ENST00000475889.1, transcript_ENST00000480227.5, transcript_ENST00000483024.1, transcript_ENST00000483558.5, transcript_ENST00000483731.5, transcript_ENST00000483814.1, transcript_ENST00000485120.1, transcript_ENST00000486339.6, transcript_ENST00000487932.5, transcript_ENST00000488185.2, transcript_ENST00000496574.6, transcript_ENST00000561668.5, transcript_ENST00000561991.5, transcript_ENST00000562297.5, transcript_ENST00000562425.1, transcript_ENST00000564313.1, transcript_ENST00000564865.5, transcript_ENST00000564890.1, transcript_ENST00000565639.6, transcript_ENST00000566784.5, transcript_ENST00000566905.5, transcript_ENST00000567355.1, transcript_ENST00000567946.1, transcript_ENST00000568591.5, transcript_ENST00000568796.1, transcript_ENST00000569983.5, transcript_ENST00000570150.1, transcript_ENST00000570193.5, and transcript_ENST00000570253.5.

In some embodiments, the PKD1 pre-mRNA transcript is encoded by a genetic sequence with at least about 80%, 85%, 90%, 95%, 96%, 97%, 98%, 99% or 100% sequence identity to the Ensembl reference number ENSG00000008710.20 or a complement thereof. In some embodiments, the PKD1 pre-mRNA transcript comprises a sequence with at least about 80%, 85%, 90%, 95%, 96%, 97%, 98%, 99% or 100% sequence identity to a PKD1 pre-mRNA transcript or a complement thereof described herein.

In some embodiments, the targeted portion of the PKD1 pre-mRNA comprises a sequence with at least 80%, 85%, 90%, 95%, 97%, or 100% sequence identity to a region comprising at least 8 contiguous nucleic acids of a sequence of premrna_ENST00000262304.9, premrna_ENST00000415938.7, premrna_ENST00000423118.5, premrna_ENST00000468674.5, premrna_ENST00000469241.2, premrna_ENST00000469851.1, premrna_ENST00000471603.6, premrna_ENST00000472577.1, premrna_ENST00000472659.1, premrna_ENST00000473780.2, premrna_ENST00000474088.1, premrna_ENST00000475889.1, premrna_ENST00000480227.5, premrna_ENST00000483024.1, premrna_ENST00000483558.5, premrna_ENST00000483731.5, premrna_ENST00000483814.1, premrna_ENST00000485120.1, premrna_ENST00000486339.6, premrna_ENST00000487932.5, premrna_ENST00000488185.2, premrna_ENST00000496574.6, premrna_ENST00000561668.5, premrna_ENST00000561991.5, premrna_ENST00000562297.5, premrna_ENST00000562425.1, premrna_ENST00000564313.1, premrna_ENST00000564865.5, premrna_ENST00000564890.1, premrna_ENST00000565639.6, premrna_ENST00000566784.5, premrna_ENST00000566905.5, premrna_ENST00000567355.1, premrna_ENST00000567946.1, premrna_ENST00000568591.5, premrna_ENST00000568796.1, premrna_ENST00000569983.5, premrna_ENST00000570150.1, premrna_ENST00000570193.5, premrna_ENST00000570253.5 or a sequence of Table 3 or complements thereof. In some embodiments, the targeted portion of the PKD1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 766-SEQ ID NO: 769 or complements thereof. In some embodiments, the targeted portion of the PKD1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PLA2G6 genomic sequence. In some embodiments, the ASO targets a NSAE pre-mRNA transcript from a PLA2G6 genomic sequence comprising a NSAE exon. In some embodiments, the ASO targets a NSAE pre-mRNA transcript from a PLA2G6 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000332509.8, premrna_ENST00000335539.7, premrna_ENST00000402064.5, premrna_ENST00000417303.6, premrna_ENST00000420435.5, premrna_ENST00000426674.1, premrna_ENST00000427114.6, premrna_ENST00000427453.5, premrna_ENST00000430886.5, premrna_ENST00000435484.5, premrna_ENST00000436218.6, premrna_ENST00000445591.5, premrna_ENST00000447598.6, premrna_ENST00000448094.5, premrna_ENST00000452542.5, premrna_ENST00000452794.5, premrna_ENST00000452972.1, premrna_ENST00000454670.1, premrna_ENST00000455341.2, premrna_ENST00000463287.1, premrna_ENST00000471636.5, premrna_ENST00000479641.5, premrna_ENST00000480154.1, premrna_ENST00000490473.1, premrna_ENST00000491986.1, premrna_ENST00000496409.1, premrna_ENST00000498338.1, premrna_ENST00000594306.1, premrna_ENST00000655142.1, premrna_ENST00000660610.1, premrna_ENST00000663895.1, premrna_ENST00000664587.1, premrna_ENST00000665987.1, premrna_ENST00000667521.1, premrna_ENST00000668208.1, premrna_ENST00000668499.1, premrna_ENST00000668949.1, premrna_ENST00000671093.1, and premrna_ENST00000673413.1. In some embodiments, the ASO targets a PLA2G6 pre-mRNA sequence comprising a NSAE exon. In some embodiments, the ASO targets a PLA2G6 pre-mRNA sequence comprising an intron flanking the 3′ splice site of the NSAE exon. In some embodiments, the ASO targets a PLA2G6 pre-mRNA sequence comprising an intron flanking the 5′ splice site of the NSAE exon. In some embodiments, the transcript is selected from the group consisting of transcript_ENST00000332509.8, transcript_ENST00000335539.7, transcript_ENST00000402064.5, transcript_ENST00000417303.6, transcript_ENST00000420435.5, transcript_ENST00000426674.1, transcript_ENST00000427114.6, transcript_ENST00000427453.5, transcript_ENST00000430886.5, transcript_ENST00000435484.5, transcript_ENST00000436218.6, transcript_ENST00000445591.5, transcript_ENST00000447598.6, transcript_ENST00000448094.5, transcript_ENST00000452542.5, transcript_ENST00000452794.5, transcript_ENST00000452972.1, transcript_ENST00000454670.1, transcript_ENST00000455341.2, transcript_ENST00000463287.1, transcript_ENST00000471636.5, transcript_ENST00000479641.5, transcript_ENST00000480154.1, transcript_ENST00000490473.1, transcript_ENST00000491986.1, transcript_ENST00000496409.1, transcript_ENST00000498338.1, transcript_ENST00000594306.1, transcript_ENST00000655142.1, transcript_ENST00000660610.1, transcript_ENST00000663895.1, transcript_ENST00000664587.1, transcript_ENST00000665987.1, transcript_ENST00000667521.1, transcript_ENST00000668208.1, transcript_ENST00000668499.1, transcript_ENST00000668949.1, transcript_ENST00000671093.1, and transcript_ENST00000673413.1.

In some embodiments, the PLA2G6 pre-mRNA transcript is encoded by a genetic sequence with at least about 80%, 85%, 90%, 95%, 96%, 97%, 98%, 99% or 100% sequence identity to the Ensembl reference number ENSG00000184381.20 or a complement thereof. In some embodiments, the PLA2G6 pre-mRNA transcript comprises a sequence with at least about 80%, 85%, 90%, 95%, 96%, 97%, 98%, 99% or 100% sequence identity to a PLA2G6 pre-mRNA transcript or a complement thereof described herein.

In some embodiments, the targeted portion of the PLA2G6 pre-mRNA comprises a sequence with at least 80%, 85%, 90%, 95%, 97%, or 100% sequence identity to a region comprising at least 8 contiguous nucleic acids of a sequence of premrna_ENST00000332509.8, premrna_ENST00000335539.7, premrna_ENST00000402064.5, premrna_ENST00000417303.6, premrna_ENST00000420435.5, premrna_ENST00000426674.1, premrna_ENST00000427114.6, premrna_ENST00000427453.5, premrna_ENST00000430886.5, premrna_ENST00000435484.5, premrna_ENST00000436218.6, premrna_ENST00000445591.5, premrna_ENST00000447598.6, premrna_ENST00000448094.5, premrna_ENST00000452542.5, premrna_ENST00000452794.5, premrna_ENST00000452972.1, premrna_ENST00000454670.1, premrna_ENST00000455341.2, premrna_ENST00000463287.1, premrna_ENST00000471636.5, premrna_ENST00000479641.5, premrna_ENST00000480154.1, premrna_ENST00000490473.1, premrna_ENST00000491986.1, premrna_ENST00000496409.1, premrna_ENST00000498338.1, premrna_ENST00000594306.1, premrna_ENST00000655142.1, premrna_ENST00000660610.1, premrna_ENST00000663895.1, premrna_ENST00000664587.1, premrna_ENST00000665987.1, premrna_ENST00000667521.1, premrna_ENST00000668208.1, premrna_ENST00000668499.1, premrna_ENST00000668949.1, premrna_ENST00000671093.1, premrna_ENST00000673413.1 or a sequence of Table 3 or complements thereof. In some embodiments, the targeted portion of the PLA2G6 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 770-SEQ ID NO: 775 or complements thereof. In some embodiments, the targeted portion of the PLA2G6 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PLD2 genomic sequence. In some embodiments, the ASO targets a NSAE pre-mRNA transcript from a PLD2 genomic sequence comprising a NSAE exon. In some embodiments, the ASO targets a NSAE pre-mRNA transcript from a PLD2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000263088.11, premrna_ENST00000571273.5, premrna_ENST00000572127.1, premrna_ENST00000572199.1, premrna_ENST00000572940.5, premrna_ENST00000573258.1, premrna_ENST00000574268.1, premrna_ENST00000574796.1, premrna_ENST00000575246.6, premrna_ENST00000575316.1, premrna_ENST00000575813.5, premrna_ENST00000575945.1, premrna_ENST00000576329.1, premrna_ENST00000576864.1, and premrna_ENST00000576983.5. In some embodiments, the ASO targets a PLD2 pre-mRNA sequence comprising a NSAE exon. In some embodiments, the ASO targets a PLD2 pre-mRNA sequence comprising an intron flanking the 3′ splice site of the NSAE exon. In some embodiments, the ASO targets a PLD2 pre-mRNA sequence comprising an intron flanking the 5′ splice site of the NSAE exon. In some embodiments, the transcript is selected from the group consisting of transcript_ENST00000263088.11, transcript_ENST00000571273.5, transcript_ENST00000572127.1, transcript_ENST00000572199.1, transcript_ENST00000572940.5, transcript_ENST00000573258.1, transcript_ENST00000574268.1, transcript_ENST00000574796.1, transcript_ENST00000575246.6, transcript_ENST00000575316.1, transcript_ENST00000575813.5, transcript_ENST00000575945.1, transcript_ENST00000576329.1, transcript_ENST00000576864.1, and transcript_ENST00000576983.5.

In some embodiments, the PLD2 pre-mRNA transcript is encoded by a genetic sequence with at least about 80%, 85%, 90%, 95%, 96%, 97%, 98%, 99% or 100% sequence identity to the Ensembl reference number ENSG00000129219.14 or a complement thereof. In some embodiments, the PLD2 pre-mRNA transcript comprises a sequence with at least about 80%, 85%, 90%, 95%, 96%, 97%, 98%, 99% or 100% sequence identity to a PLD2 pre-mRNA transcript or a complement thereof described herein.

In some embodiments, the targeted portion of the PLD2 pre-mRNA comprises a sequence with at least 80%, 85%, 90%, 95%, 97%, or 100% sequence identity to a region comprising at least 8 contiguous nucleic acids of a sequence of premrna_ENST00000263088.11, premrna_ENST00000571273.5, premrna_ENST00000572127.1, premrna_ENST00000572199.1, premrna_ENST00000572940.5, premrna_ENST00000573258.1, premrna_ENST00000574268.1, premrna_ENST00000574796.1, premrna_ENST00000575246.6, premrna_ENST00000575316.1, premrna_ENST00000575813.5, premrna_ENST00000575945.1, premrna_ENST00000576329.1, premrna_ENST00000576864.1, premrna_ENST00000576983.5 or a sequence of Table 3 or complements thereof. In some embodiments, the targeted portion of the PLD2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 776-SEQ ID NO: 787 or complements thereof. In some embodiments, the targeted portion of the PLD2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PLEKHG5 genomic sequence. In some embodiments, the ASO targets a NSAE pre-mRNA transcript from a PLEKHG5 genomic sequence comprising a NSAE exon. In some embodiments, the ASO targets a NSAE pre-mRNA transcript from a PLEKHG5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000340850.9, premrna_ENST00000377725.5, premrna_ENST00000377728.7, premrna_ENST00000377732.5, premrna_ENST00000377740.4, premrna_ENST00000377748.5, premrna_ENST00000400913.5, premrna_ENST00000400915.7, premrna_ENST00000487949.4, premrna_ENST00000489097.5, premrna_ENST00000535355.5, premrna_ENST00000537245.5, and premrna_ENST00000673471.1. In some embodiments, the ASO targets a PLEKHG5 pre-mRNA sequence comprising a NSAE exon. In some embodiments, the ASO targets a PLEKHG5 pre-mRNA sequence comprising an intron flanking the 3′ splice site of the NSAE exon. In some embodiments, the ASO targets a PLEKHG5 pre-mRNA sequence comprising an intron flanking the 5′ splice site of the NSAE exon. In some embodiments, the transcript is selected from the group consisting of transcript_ENST00000340850.9, transcript_ENST00000377725.5, transcript_ENST00000377728.7, transcript_ENST00000377732.5, transcript_ENST00000377740.4, transcript_ENST00000377748.5, transcript_ENST00000400913.5, transcript_ENST00000400915.7, transcript_ENST00000487949.4, transcript_ENST00000489097.5, transcript_ENST00000535355.5, transcript_ENST00000537245.5, and transcript_ENST00000673471.1.

In some embodiments, the PLEKHG5 pre-mRNA transcript is encoded by a genetic sequence with at least about 80%, 85%, 90%, 95%, 96%, 97%, 98%, 99% or 100% sequence identity to the Ensembl reference number ENSG00000171680.22 or a complement thereof. In some embodiments, the PLEKHG5 pre-mRNA transcript comprises a sequence with at least about 80%, 85%, 90%, 95%, 96%, 97%, 98%, 99% or 100% sequence identity to a PLEKHG5 pre-mRNA transcript or a complement thereof described herein.

In some embodiments, the targeted portion of the PLEKHG5 pre-mRNA comprises a sequence with at least 80%, 85%, 90%, 95%, 97%, or 100% sequence identity to a region comprising at least 8 contiguous nucleic acids of a sequence of premrna_ENST00000340850.9, premrna_ENST00000377725.5, premrna_ENST00000377728.7, premrna_ENST00000377732.5, premrna_ENST00000377740.4, premrna_ENST00000377748.5, premrna_ENST00000400913.5, premrna_ENST00000400915.7, premrna_ENST00000487949.4, premrna_ENST00000489097.5, premrna_ENST00000535355.5, premrna_ENST00000537245.5, premrna_ENST00000673471.1 or a sequence of Table 3 or complements thereof. In some embodiments, the targeted portion of the PLEKHG5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 788-SEQ ID NO: 791 or complements thereof. In some embodiments, the targeted portion of the PLEKHG5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PNPO genomic sequence. In some embodiments, the ASO targets a NSAE pre-mRNA transcript from a PNPO genomic sequence comprising a NSAE exon. In some embodiments, the ASO targets a NSAE pre-mRNA transcript from a PNPO genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000225573.5, premrna_ENST00000434554.7, premrna_ENST00000582171.6, premrna_ENST00000583245.6, premrna_ENST00000583599.6, premrna_ENST00000584061.6, premrna_ENST00000584806.2, premrna_ENST00000585320.5, premrna_ENST00000641285.1, premrna_ENST00000641305.1, premrna_ENST00000641323.1, premrna_ENST00000641427.1, premrna_ENST00000641511.1, premrna_ENST00000641703.1, premrna_ENST00000641709.1, premrna_ENST00000641856.1, and premrna_ENST00000642017.1. In some embodiments, the ASO targets a PNPO pre-mRNA sequence comprising a NSAE exon. In some embodiments, the ASO targets a PNPO pre-mRNA sequence comprising an intron flanking the 3′ splice site of the NSAE exon. In some embodiments, the ASO targets a PNPO pre-mRNA sequence comprising an intron flanking the 5′ splice site of the NSAE exon. In some embodiments, the transcript is selected from the group consisting of transcript_ENST00000225573.5, transcript_ENST00000434554.7, transcript_ENST00000582171.6, transcript_ENST00000583245.6, transcript_ENST00000583599.6, transcript_ENST00000584061.6, transcript_ENST00000584806.2, transcript_ENST00000585320.5, transcript_ENST00000641285.1, transcript_ENST00000641305.1, transcript_ENST00000641323.1, transcript_ENST00000641427.1, transcript_ENST00000641511.1, transcript_ENST00000641703.1, transcript_ENST00000641709.1, transcript_ENST00000641856.1, and transcript_ENST00000642017.1.

In some embodiments, the PNPO pre-mRNA transcript is encoded by a genetic sequence with at least about 80%, 85%, 90%, 95%, 96%, 97%, 98%, 99% or 100% sequence identity to the Ensembl reference number ENSG00000108439.11 or a complement thereof. In some embodiments, the PNPO pre-mRNA transcript comprises a sequence with at least about 80%, 85%, 90%, 95%, 96%, 97%, 98%, 99% or 100% sequence identity to a PNPO pre-mRNA transcript or a complement thereof described herein.

In some embodiments, the targeted portion of the PNPO pre-mRNA comprises a sequence with at least 80%, 85%, 90%, 95%, 97%, or 100% sequence identity to a region comprising at least 8 contiguous nucleic acids of a sequence of premrna_ENST00000225573.5, premrna_ENST00000434554.7, premrna_ENST00000582171.6, premrna_ENST00000583245.6, premrna_ENST00000583599.6, premrna_ENST00000584061.6, premrna_ENST00000584806.2, premrna_ENST00000585320.5, premrna_ENST00000641285.1, premrna_ENST00000641305.1, premrna_ENST00000641323.1, premrna_ENST00000641427.1, premrna_ENST00000641511.1, premrna_ENST00000641703.1, premrna_ENST00000641709.1, premrna_ENST00000641856.1, premrna_ENST00000642017.1, or a sequence of Table 3 or complements thereof. In some embodiments, the targeted portion of the PNPO pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 792-SEQ ID NO: 803 or complements thereof. In some embodiments, the targeted portion of the PNPO pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a POLE genomic sequence. In some embodiments, the ASO targets a NSAE pre-mRNA transcript from a POLE genomic sequence comprising a NSAE exon. In some embodiments, the ASO targets a NSAE pre-mRNA transcript from a POLE genomic sequence comprising an intron flanking the 3′ splice site of the NSAE exon and an intron flanking the 5′ splice site of a NSAE exon. In some embodiments, the ASO targets a POLE pre-mRNA sequence comprising a NSAE exon. In some embodiments, the ASO targets a POLE pre-mRNA sequence comprising an intron flanking the 3′ splice site of the NSAE exon. In some embodiments, the ASO targets a POLE pre-mRNA sequence comprising an intron flanking the 5′ splice site of the NSAE exon.

In some embodiments, the POLE pre-mRNA transcript is encoded by a genetic sequence with at least about 80%, 85%, 90%, 95%, 96%, 97%, 98%, 99% or 100% sequence identity to the Ensembl reference number ENSG00000177084.18 or a complement thereof. In some embodiments, the POLE pre-mRNA transcript comprises a sequence with at least about 80%, 85%, 90%, 95%, 96%, 97%, 98%, 99% or 100% sequence identity to a POLE pre-mRNA transcript or a complement thereof described herein.

In some embodiments, the targeted portion of the POLE pre-mRNA comprises a sequence with at least 80%, 85%, 90%, 95%, 97%, or 100% sequence identity to a region comprising at least 8 contiguous nucleic acids of a sequence of Table 3 or complements thereof. In some embodiments, the targeted portion of the POLE pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 804-SEQ ID NO: 807 or complements thereof. In some embodiments, the targeted portion of the POLE pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PON2 genomic sequence. In some embodiments, the ASO targets a NSAE pre-mRNA transcript from a PON2 genomic sequence comprising a NSAE exon. In some embodiments, the ASO targets a NSAE pre-mRNA transcript from a PON2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000222572.8, premrna_ENST00000433091.6, premrna_ENST00000446142.5, premrna_ENST00000455123.5, premrna_ENST00000459842.1, premrna_ENST00000460873.5, premrna_ENST00000469716.1, premrna_ENST00000469926.5, premrna_ENST00000471883.1, premrna_ENST00000478801.5, premrna_ENST00000483292.5, premrna_ENST00000490778.5, premrna_ENST00000491069.5, premrna_ENST00000493290.5, premrna_ENST00000493469.5, premrna_ENST00000632034.1, premrna_ENST00000633192.1, and premrna_ENST00000633531.1. In some embodiments, the ASO targets a PON2 pre-mRNA sequence comprising a NSAE exon. In some embodiments, the ASO targets a PON2 pre-mRNA sequence comprising an intron flanking the 3′ splice site of the NSAE exon. In some embodiments, the ASO targets a PON2 pre-mRNA sequence comprising an intron flanking the 5′ splice site of the NSAE exon. In some embodiments, the transcript is selected from the group consisting of transcript_ENST00000222572.8, transcript_ENST00000433091.6, transcript_ENST00000446142.5, transcript_ENST00000455123.5, transcript_ENST00000459842.1, transcript_ENST00000460873.5, transcript_ENST00000469716.1, transcript_ENST00000469926.5, transcript_ENST00000471883.1, transcript_ENST00000478801.5, transcript_ENST00000483292.5, transcript_ENST00000490778.5, transcript_ENST00000491069.5, transcript_ENST00000493290.5, transcript_ENST00000493469.5, transcript_ENST00000632034.1, transcript_ENST00000633192.1, and transcript_ENST00000633531.1.

In some embodiments, the PON2 pre-mRNA transcript is encoded by a genetic sequence with at least about 80%, 85%, 90%, 95%, 96%, 97%, 98%, 99% or 100% sequence identity to the Ensembl reference number ENSG00000105854.13 or a complement thereof. In some embodiments, the PON2 pre-mRNA transcript comprises a sequence with at least about 80%, 85%, 90%, 95%, 96%, 97%, 98%, 99% or 100% sequence identity to a PON2 pre-mRNA transcript or a complement thereof described herein.

In some embodiments, the targeted portion of the PON2 pre-mRNA comprises a sequence with at least 80%, 85%, 90%, 95%, 97%, or 100% sequence identity to a region comprising at least 8 contiguous nucleic acids of a sequence of premrna_ENST00000222572.8, premrna_ENST00000433091.6, premrna_ENST00000446142.5, premrna_ENST00000455123.5, premrna_ENST00000459842.1, premrna_ENST00000460873.5, premrna_ENST00000469716.1, premrna_ENST00000469926.5, premrna_ENST00000471883.1, premrna_ENST00000478801.5, premrna_ENST00000483292.5, premrna_ENST00000490778.5, premrna_ENST00000491069.5, premrna_ENST00000493290.5, premrna_ENST00000493469.5, premrna_ENST00000632034.1, premrna_ENST00000633192.1, premrna_ENST00000633531.1 or a sequence of Table 3 or complements thereof. In some embodiments, the targeted portion of the PON2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 808-SEQ ID NO: 811 or complements thereof. In some embodiments, the targeted portion of the PON2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PRMT7 genomic sequence. In some embodiments, the ASO targets a NSAE pre-mRNA transcript from a PRMT7 genomic sequence comprising a NSAE exon. In some embodiments, the ASO targets a NSAE pre-mRNA transcript from a PRMT7 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000339507.9, premrna_ENST00000441236.2, premrna_ENST00000449359.7, premrna_ENST00000561806.5, premrna_ENST00000562050.5, premrna_ENST00000562381.7, premrna_ENST00000562456.1, premrna_ENST00000563443.1, premrna_ENST00000563520.5, premrna_ENST00000563562.5, premrna_ENST00000563608.2, premrna_ENST00000564050.6, premrna_ENST00000564441.5, premrna_ENST00000565356.5, premrna_ENST00000565745.5, premrna_ENST00000565761.1, premrna_ENST00000565983.5, premrna_ENST00000566341.5, premrna_ENST00000566430.2, premrna_ENST00000566657.5, premrna_ENST00000566687.1, premrna_ENST00000566708.1, premrna_ENST00000567542.5, premrna_ENST00000568463.1, premrna_ENST00000568975.5, premrna_ENST00000569047.7, and premrna_ENST00000569571.5. In some embodiments, the ASO targets a PRMT7 pre-mRNA sequence comprising a NSAE exon. In some embodiments, the ASO targets a PRMT7 pre-mRNA sequence comprising an intron flanking the 3′ splice site of the NSAE exon. In some embodiments, the ASO targets a PRMT7 pre-mRNA sequence comprising an intron flanking the 5′ splice site of the NSAE exon. In some embodiments, the transcript is selected from the group consisting of transcript_ENST00000339507.9, transcript_ENST00000441236.2, transcript_ENST00000449359.7, transcript_ENST00000561806.5, transcript_ENST00000562050.5, transcript_ENST00000562381.7, transcript_ENST00000562456.1, transcript_ENST00000563443.1, transcript_ENST00000563520.5, transcript_ENST00000563562.5, transcript_ENST00000563608.2, transcript_ENST00000564050.6, transcript_ENST00000564441.5, transcript_ENST00000565356.5, transcript_ENST00000565745.5, transcript_ENST00000565761.1, transcript_ENST00000565983.5, transcript_ENST00000566341.5, transcript_ENST00000566430.2, transcript_ENST00000566657.5, transcript_ENST00000566687.1, transcript_ENST00000566708.1, transcript_ENST00000567542.5, transcript_ENST00000568463.1, transcript_ENST00000568975.5, transcript_ENST00000569047.7, and transcript_ENST00000569571.5.

In some embodiments, the PRMT7 pre-mRNA transcript is encoded by a genetic sequence with at least about 80%, 85%, 90%, 95%, 96%, 97%, 98%, 99% or 100% sequence identity to the Ensembl reference number ENSG00000132600.16 or a complement thereof. In some embodiments, the PRMT7 pre-mRNA transcript comprises a sequence with at least about 80%, 85%, 90%, 95%, 96%, 97%, 98%, 99% or 100% sequence identity to a PRMT7 pre-mRNA transcript or a complement thereof described herein.

In some embodiments, the targeted portion of the PRMT7 pre-mRNA comprises a sequence with at least 80%, 85%, 90%, 95%, 97%, or 100% sequence identity to a region comprising at least 8 contiguous nucleic acids of a sequence of premrna_ENST00000339507.9, premrna_ENST00000441236.2, premrna_ENST00000449359.7, premrna_ENST00000561806.5, premrna_ENST00000562050.5, premrna_ENST00000562381.7, premrna_ENST00000562456.1, premrna_ENST00000563443.1, premrna_ENST00000563520.5, premrna_ENST00000563562.5, premrna_ENST00000563608.2, premrna_ENST00000564050.6, premrna_ENST00000564441.5, premrna_ENST00000565356.5, premrna_ENST00000565745.5, premrna_ENST00000565761.1, premrna_ENST00000565983.5, premrna_ENST00000566341.5, premrna_ENST00000566430.2, premrna_ENST00000566657.5, premrna_ENST00000566687.1, premrna_ENST00000566708.1, premrna_ENST00000567542.5, premrna_ENST00000568463.1, premrna_ENST00000568975.5, premrna_ENST00000569047.7, premrna_ENST00000569571.5 or a sequence of Table 3 or complements thereof. In some embodiments, the targeted portion of the PRMT7 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 812-SEQ ID NO: 815 or complements thereof. In some embodiments, the targeted portion of the PRMT7 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PRODH genomic sequence. In some embodiments, the ASO targets a NSAE pre-mRNA transcript from a PRODH genomic sequence comprising a NSAE exon. In some embodiments, the ASO targets a NSAE pre-mRNA transcript from a PRODH genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000313755.9, premrna_ENST00000334029.6, premrna_ENST00000357068.10, premrna_ENST00000399694.1, premrna_ENST00000420436.5, premrna_ENST00000429300.5, premrna_ENST00000438924.5, premrna_ENST00000446371.1, premrna_ENST00000450579.1, premrna_ENST00000457083.1, premrna_ENST00000482858.5, premrna_ENST00000491604.5, premrna_ENST00000496625.1, premrna_ENST00000609229.1, and premrna_ENST00000610940.4. In some embodiments, the ASO targets a PRODH pre-mRNA sequence comprising a NSAE exon. In some embodiments, the ASO targets a PRODH pre-mRNA sequence comprising an intron flanking the 3′ splice site of the NSAE exon. In some embodiments, the ASO targets a PRODH pre-mRNA sequence comprising an intron flanking the 5′ splice site of the NSAE exon. In some embodiments, the transcript is selected from the group consisting of transcript_ENST00000313755.9, transcript_ENST00000334029.6, transcript_ENST00000357068.10, transcript_ENST00000399694.1, transcript_ENST00000420436.5, transcript_ENST00000429300.5, transcript_ENST00000438924.5, transcript_ENST00000446371.1, transcript_ENST00000450579.1, transcript_ENST00000457083.1, transcript_ENST00000482858.5, transcript_ENST00000491604.5, transcript_ENST00000496625.1, transcript_ENST00000609229.1, and transcript_ENST00000610940.4.

In some embodiments, the PRODH pre-mRNA transcript is encoded by a genetic sequence with at least about 80%, 85%, 90%, 95%, 96%, 97%, 98%, 99% or 100% sequence identity to the Ensembl reference number ENSG00000100033.16 or a complement thereof. In some embodiments, the PRODH pre-mRNA transcript comprises a sequence with at least about 80%, 85%, 90%, 95%, 96%, 97%, 98%, 99% or 100% sequence identity to a PRODH pre-mRNA transcript or a complement thereof described herein.

In some embodiments, the targeted portion of the PRODH pre-mRNA comprises a sequence with at least 80%, 85%, 90%, 95%, 97%, or 100% sequence identity to a region comprising at least 8 contiguous nucleic acids of a sequence of premrna_ENST00000313755.9, premrna_ENST00000334029.6, premrna_ENST00000357068.10, premrna_ENST00000399694.1, premrna_ENST00000420436.5, premrna_ENST00000429300.5, premrna_ENST00000438924.5, premrna_ENST00000446371.1, premrna_ENST00000450579.1, premrna_ENST00000457083.1, premrna_ENST00000482858.5, premrna_ENST00000491604.5, premrna_ENST00000496625.1, premrna_ENST00000609229.1, premrna_ENST00000610940.4, or a sequence of Table 3 or complements thereof. In some embodiments, the targeted portion of the PRODH pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 816-SEQ ID NO: 819 or complements thereof. In some embodiments, the targeted portion of the PRODH pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PRPF3 genomic sequence. In some embodiments, the ASO targets a NSAE pre-mRNA transcript from a PRPF3 genomic sequence comprising a NSAE exon. In some embodiments, the ASO targets a NSAE pre-mRNA transcript from a PRPF3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000324862.7, premrna_ENST00000467329.5, premrna_ENST00000467514.1, premrna_ENST00000470824.1, premrna_ENST00000476970.1, premrna_ENST00000493553.1, and premrna_ENST00000496202.5. In some embodiments, the ASO targets a PRPF3 pre-mRNA sequence comprising a NSAE exon. In some embodiments, the ASO targets a PRPF3 pre-mRNA sequence comprising an intron flanking the 3′ splice site of the NSAE exon. In some embodiments, the ASO targets a PRPF3 pre-mRNA sequence comprising an intron flanking the 5′ splice site of the NSAE exon. In some embodiments, the transcript is selected from the group consisting of transcript_ENST00000324862.7, transcript_ENST00000467329.5, transcript_ENST00000467514.1, transcript_ENST00000470824.1, transcript_ENST00000476970.1, transcript_ENST00000493553.1, and transcript_ENST00000496202.5.

In some embodiments, the PRPF3 pre-mRNA transcript is encoded by a genetic sequence with at least about 80%, 85%, 90%, 95%, 96%, 97%, 98%, 99% or 100% sequence identity to the Ensembl reference number ENSG00000117360.13 or a complement thereof. In some embodiments, the PRPF3 pre-mRNA transcript comprises a sequence with at least about 80%, 85%, 90%, 95%, 96%, 97%, 98%, 99% or 100% sequence identity to a PRPF3 pre-mRNA transcript or a complement thereof described herein.

In some embodiments, the targeted portion of the PRPF3 pre-mRNA comprises a sequence with at least 80%, 85%, 90%, 95%, 97%, or 100% sequence identity to a region comprising at least 8 contiguous nucleic acids of a sequence of premrna_ENST00000324862.7, premrna_ENST00000467329.5, premrna_ENST00000467514.1, premrna_ENST00000470824.1, premrna_ENST00000476970.1, premrna_ENST00000493553.1, premrna_ENST00000496202.5, or a sequence of Table 3 or complements thereof. In some embodiments, the targeted portion of the PRPF3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 820-SEQ ID NO: 823 or complements thereof. In some embodiments, the targeted portion of the PRPF3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PRPF4 genomic sequence. In some embodiments, the ASO targets a NSAE pre-mRNA transcript from a PRPF4 genomic sequence comprising a NSAE exon. In some embodiments, the ASO targets a NSAE pre-mRNA transcript from a PRPF4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000374198.4, premrna_ENST00000374199.8, and premrna_ENST00000488937.1. In some embodiments, the ASO targets a PRPF4 pre-mRNA sequence comprising a NSAE exon. In some embodiments, the ASO targets a PRPF4 pre-mRNA sequence comprising an intron flanking the 3′ splice site of the NSAE exon. In some embodiments, the ASO targets a PRPF4 pre-mRNA sequence comprising an intron flanking the 5′ splice site of the NSAE exon. In some embodiments, the transcript is selected from the group consisting of transcript_ENST00000374198.4, transcript_ENST00000374199.8, and transcript_ENST00000488937.1

In some embodiments, the PRPF4 pre-mRNA transcript is encoded by a genetic sequence with at least about 80%, 85%, 90%, 95%, 96%, 97%, 98%, 99% or 100% sequence identity to the Ensembl reference number ENSG00000136875.13 or a complement thereof. In some embodiments, the PRPF4 pre-mRNA transcript comprises a sequence with at least about 80%, 85%, 90%, 95%, 96%, 97%, 98%, 99% or 100% sequence identity to a PRPF4 pre-mRNA transcript or a complement thereof described herein.

In some embodiments, the targeted portion of the PRPF4 pre-mRNA comprises a sequence with at least 80%, 85%, 90%, 95%, 97%, or 100% sequence identity to a region comprising at least 8 contiguous nucleic acids of a sequence of premrna_ENST00000374198.4, premrna_ENST00000374199.8, premrna_ENST00000488937.1, or a sequence of Table 3 or complements thereof. In some embodiments, the targeted portion of the PRPF4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 824-SEQ ID NO: 827 or complements thereof. In some embodiments, the targeted portion of the PRPF4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a PYCR1 genomic sequence. In some embodiments, the ASO targets a NSAE pre-mRNA transcript from a PYCR1 genomic sequence comprising a NSAE exon. In some embodiments, the ASO targets a NSAE pre-mRNA transcript from a PYCR1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000329875.13, premrna_ENST00000337943.9, premrna_ENST00000402252.6, premrna_ENST00000403172.8, premrna_ENST00000405481.8, premrna_ENST00000577624.5, premrna_ENST00000577756.5, premrna_ENST00000579366.5, premrna_ENST00000579698.5, premrna_ENST00000581271.5, premrna_ENST00000582198.5, premrna_ENST00000583564.5, premrna_ENST00000584848.5, premrna_ENST00000585215.5, premrna_ENST00000585244.1, premrna_ENST00000619204.4, and premrna_ENST00000629768.2. In some embodiments, the ASO targets a PYCR1 pre-mRNA sequence comprising a NSAE exon. In some embodiments, the ASO targets a PYCR1 pre-mRNA sequence comprising an intron flanking the 3′ splice site of the NSAE exon. In some embodiments, the ASO targets a PYCR1 pre-mRNA sequence comprising an intron flanking the 5′ splice site of the NSAE exon. In some embodiments, the transcript is selected from the group consisting of transcript_ENST00000329875.13, transcript_ENST00000337943.9, transcript_ENST00000402252.6, transcript_ENST00000403172.8, transcript_ENST00000405481.8, transcript_ENST00000577624.5, transcript_ENST00000577756.5, transcript_ENST00000579366.5, transcript_ENST00000579698.5, transcript_ENST00000581271.5, transcript_ENST00000582198.5, transcript_ENST00000583564.5, transcript_ENST00000584848.5, transcript_ENST00000585215.5, transcript_ENST00000585244.1, transcript_ENST00000619204.4, and transcript_ENST00000629768.2.

In some embodiments, the PYCR1 pre-mRNA transcript is encoded by a genetic sequence with at least about 80%, 85%, 90%, 95%, 96%, 97%, 98%, 99% or 100% sequence identity to the Ensembl reference number ENSG00000183010.17 or a complement thereof. In some embodiments, the PYCR1 pre-mRNA transcript comprises a sequence with at least about 80%, 85%, 90%, 95%, 96%, 97%, 98%, 99% or 100% sequence identity to a PYCR1 pre-mRNA transcript or a complement thereof described herein.

In some embodiments, the targeted portion of the PYCR1 pre-mRNA comprises a sequence with at least 80%, 85%, 90%, 95%, 97%, or 100% sequence identity to a region comprising at least 8 contiguous nucleic acids of a sequence of premrna_ENST00000329875.13, premrna_ENST00000337943.9, premrna_ENST00000402252.6, premrna_ENST00000403172.8, premrna_ENST00000405481.8, premrna_ENST00000577624.5, premrna_ENST00000577756.5, premrna_ENST00000579366.5, premrna_ENST00000579698.5, premrna_ENST00000581271.5, premrna_ENST00000582198.5, premrna_ENST00000583564.5, premrna_ENST00000584848.5, premrna_ENST00000585215.5, premrna_ENST00000585244.1, premrna_ENST00000619204.4, premrna_ENST00000629768.2 or a sequence of Table 3 or complements thereof. In some embodiments, the targeted portion of the PYCR1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 828-SEQ ID NO: 831 or complements thereof. In some embodiments, the targeted portion of the PYCR1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a RAD52 genomic sequence. In some embodiments, the ASO targets a NSAE pre-mRNA transcript from a RAD52 genomic sequence comprising a NSAE exon. In some embodiments, the ASO targets a NSAE pre-mRNA transcript from a RAD52 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000228345.9, premrna_ENST00000358495.8, premrna_ENST00000397230.6, premrna_ENST00000430095.6, premrna_ENST00000461568.5, premrna_ENST00000463750.5, premrna_ENST00000468231.5, premrna_ENST00000481052.5, premrna_ENST00000488642.6, premrna_ENST00000535376.5, premrna_ENST00000536177.5, premrna_ENST00000541619.1, premrna_ENST00000542297.1, premrna_ENST00000542584.5, premrna_ENST00000542785.5, premrna_ENST00000543912.5, premrna_ENST00000544742.5, premrna_ENST00000545564.5, and premrna_ENST00000545967.1.

In some embodiments, the ASO targets a RAD52 pre-mRNA sequence comprising a NSAE exon. In some embodiments, the ASO targets a RAD52 pre-mRNA sequence comprising an intron flanking the 3′ splice site of the NSAE exon. In some embodiments, the ASO targets a RAD52 pre-mRNA sequence comprising an intron flanking the 5′ splice site of the NSAE exon. In some embodiments, the transcript is selected from the group consisting of transcript_ENST00000228345.9, transcript_ENST00000358495.8, transcript_ENST00000397230.6, transcript_ENST00000430095.6, transcript_ENST00000461568.5, transcript_ENST00000463750.5, transcript_ENST00000468231.5, transcript_ENST00000481052.5, transcript_ENST00000488642.6, transcript_ENST00000535376.5, transcript_ENST00000536177.5, transcript_ENST00000541619.1, transcript_ENST00000542297.1, transcript_ENST00000542584.5, transcript_ENST00000542785.5, transcript_ENST00000543912.5, transcript_ENST00000544742.5, transcript_ENST00000545564.5, and transcript_ENST00000545967.1.

In some embodiments, the RAD52 pre-mRNA transcript is encoded by a genetic sequence with at least about 80%, 85%, 90%, 95%, 96%, 97%, 98%, 99% or 100% sequence identity to the Ensembl reference number ENSG00000002016.18 or a complement thereof. In some embodiments, the RAD52 pre-mRNA transcript comprises a sequence with at least about 80%, 85%, 90%, 95%, 96%, 97%, 98%, 99% or 100% sequence identity to a RAD52 pre-mRNA transcript or a complement thereof described herein.

In some embodiments, the targeted portion of the RAD52 pre-mRNA comprises a sequence with at least 80%, 85%, 90%, 95%, 97%, or 100% sequence identity to a region comprising at least 8 contiguous nucleic acids of a sequence of premrna_ENST00000228345.9, premrna_ENST00000358495.8, premrna_ENST00000397230.6, premrna_ENST00000430095.6, premrna_ENST00000461568.5, premrna_ENST00000463750.5, premrna_ENST00000468231.5, premrna_ENST00000481052.5, premrna_ENST00000488642.6, premrna_ENST00000535376.5, premrna_ENST00000536177.5, premrna_ENST00000541619.1, premrna_ENST00000542297.1, premrna_ENST00000542584.5, premrna_ENST00000542785.5, premrna_ENST00000543912.5, premrna_ENST00000544742.5, premrna_ENST00000545564.5, premrna_ENST00000545967.1, or a sequence of Table 3 or complements thereof. In some embodiments, the targeted portion of the RAD52 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 832-SEQ ID NO: 839 or complements thereof. In some embodiments, the targeted portion of the RAD52 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a REXO1 genomic sequence. In some embodiments, the ASO targets a NSAE pre-mRNA transcript from a REXO1 genomic sequence comprising a NSAE exon. In some embodiments, the ASO targets a NSAE pre-mRNA transcript from a REXO1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000170168.9, premrna_ENST00000586291.1, premrna_ENST00000586343.2, premrna_ENST00000587404.1, premrna_ENST00000587524.1, premrna_ENST00000588743.2, premrna_ENST00000590936.5, and premrna_ENST00000643515.1. In some embodiments, the ASO targets a REXO1 pre-mRNA sequence comprising a NSAE exon. In some embodiments, the ASO targets a REXO1 pre-mRNA sequence comprising an intron flanking the 3′ splice site of the NSAE exon. In some embodiments, the ASO targets a REXO1 pre-mRNA sequence comprising an intron flanking the 5′ splice site of the NSAE exon. In some embodiments, the transcript is selected from the group consisting of transcript_ENST00000170168.9, transcript_ENST00000586291.1, transcript_ENST00000586343.2, transcript_ENST00000587404.1, transcript_ENST00000587524.1, transcript_ENST00000588743.2, transcript_ENST00000590936.5, and transcript_ENST00000643515.1.

In some embodiments, the REXO1 pre-mRNA transcript is encoded by a genetic sequence with at least about 80%, 85%, 90%, 95%, 96%, 97%, 98%, 99% or 100% sequence identity to the Ensembl reference number ENSG00000079313.15 or a complement thereof. In some embodiments, the REXO1 pre-mRNA transcript comprises a sequence with at least about 80%, 85%, 90%, 95%, 96%, 97%, 98%, 99% or 100% sequence identity to a REXO1 pre-mRNA transcript or a complement thereof described herein.

In some embodiments, the targeted portion of the REXO1 pre-mRNA comprises a sequence with at least 80%, 85%, 90%, 95%, 97%, or 100% sequence identity to a region comprising at least 8 contiguous nucleic acids of a sequence of premrna_ENST00000170168.9, premrna_ENST00000586291.1, premrna_ENST00000586343.2, premrna_ENST00000587404.1, premrna_ENST00000587524.1, premrna_ENST00000588743.2, premrna_ENST00000590936.5, premrna_ENST00000643515.1 or a sequence of Table 3 or complements thereof. In some embodiments, the targeted portion of the REXO1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 840-SEQ ID NO: 847 or complements thereof. In some embodiments, the targeted portion of the REXO1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a RFX5 genomic sequence. In some embodiments, the ASO targets a NSAE pre-mRNA transcript from a RFX5 genomic sequence comprising a NSAE exon. In some embodiments, the ASO targets a NSAE pre-mRNA transcript from a RFX5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000290524.8, premrna_ENST00000368870.6, premrna_ENST00000392746.7, premrna_ENST00000412774.5, premrna_ENST00000421986.5, premrna_ENST00000422595.5, premrna_ENST00000430227.5, premrna_ENST00000435314.5, premrna_ENST00000436271.5, premrna_ENST00000436637.5, premrna_ENST00000437327.5, premrna_ENST00000444392.5, premrna_ENST00000450506.5, premrna_ENST00000452456.1, premrna_ENST00000452671.6, premrna_ENST00000458484.5, premrna_ENST00000469513.5, premrna_ENST00000475144.1, premrna_ENST00000478564.5, premrna_ENST00000479681.5, and premrna_ENST00000494217.5. In some embodiments, the ASO targets a RFX5 pre-mRNA sequence comprising a NSAE exon. In some embodiments, the ASO targets a RFX5 pre-mRNA sequence comprising an intron flanking the 3′ splice site of the NSAE exon. In some embodiments, the ASO targets a RFX5 pre-mRNA sequence comprising an intron flanking the 5′ splice site of the NSAE exon. In some embodiments, the transcript is selected from the group consisting of transcript_ENST00000290524.8, transcript_ENST00000368870.6, transcript_ENST00000392746.7, transcript_ENST00000412774.5, transcript_ENST00000421986.5, transcript_ENST00000422595.5, transcript_ENST00000430227.5, transcript_ENST00000435314.5, transcript_ENST00000436271.5, transcript_ENST00000436637.5, transcript_ENST00000437327.5, transcript_ENST00000444392.5, transcript_ENST00000450506.5, transcript_ENST00000452456.1, transcript_ENST00000452671.6, transcript_ENST00000458484.5, transcript_ENST00000469513.5, transcript_ENST00000475144.1, transcript_ENST00000478564.5, transcript_ENST00000479681.5, and transcript_ENST00000494217.5.

In some embodiments, the RFX5 pre-mRNA transcript is encoded by a genetic sequence with at least about 80%, 85%, 90%, 95%, 96%, 97%, 98%, 99% or 100% sequence identity to the Ensembl reference number ENSG00000143390.17 or a complement thereof. In some embodiments, the RFX5 pre-mRNA transcript comprises a sequence with at least about 80%, 85%, 90%, 95%, 96%, 97%, 98%, 99% or 100% sequence identity to a RFX5 pre-mRNA transcript or a complement thereof described herein.

In some embodiments, the targeted portion of the RFX5 pre-mRNA comprises a sequence with at least 80%, 85%, 90%, 95%, 97%, or 100% sequence identity to a region comprising at least 8 contiguous nucleic acids of a sequence of premrna_ENST00000290524.8, premrna_ENST00000368870.6, premrna_ENST00000392746.7, premrna_ENST00000412774.5, premrna_ENST00000421986.5, premrna_ENST00000422595.5, premrna_ENST00000430227.5, premrna_ENST00000435314.5, premrna_ENST00000436271.5, premrna_ENST00000436637.5, premrna_ENST00000437327.5, premrna_ENST00000444392.5, premrna_ENST00000450506.5, premrna_ENST00000452456.1, premrna_ENST00000452671.6, premrna_ENST00000458484.5, premrna_ENST00000469513.5, premrna_ENST00000475144.1, premrna_ENST00000478564.5, premrna_ENST00000479681.5, premrna_ENST00000494217.5 or a sequence of Table 3 or complements thereof. In some embodiments, the targeted portion of the RFX5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 848-SEQ ID NO: 853 or complements thereof. In some embodiments, the targeted portion of the RFX5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a RHBDF2 genomic sequence. In some embodiments, the ASO targets a NSAE pre-mRNA transcript from a RHBDF2 genomic sequence comprising a NSAE exon. In some embodiments, the ASO targets a NSAE pre-mRNA transcript from a RHBDF2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000313080.8, premrna_ENST00000585701.5, premrna_ENST00000585989.5, premrna_ENST00000587640.1, premrna_ENST00000589526.5, premrna_ENST00000589582.5, premrna_ENST00000590168.5, premrna_ENST00000590288.1, premrna_ENST00000590322.1, premrna_ENST00000591192.1, premrna_ENST00000591255.5, premrna_ENST00000591697.5, premrna_ENST00000591860.1, premrna_ENST00000591879.1, premrna_ENST00000591885.5, premrna_ENST00000592123.5, premrna_ENST00000592378.5, and premrna_ENST00000593103.1. In some embodiments, the ASO targets a RHBDF2 pre-mRNA sequence comprising a NSAE exon. In some embodiments, the ASO targets a RHBDF2 pre-mRNA sequence comprising an intron flanking the 3′ splice site of the NSAE exon. In some embodiments, the ASO targets a RHBDF2 pre-mRNA sequence comprising an intron flanking the 5′ splice site of the NSAE exon. In some embodiments, the transcript is selected from the group consisting of transcript_ENST00000313080.8, transcript_ENST00000585701.5, transcript_ENST00000585989.5, transcript_ENST00000587640.1, transcript_ENST00000589526.5, transcript_ENST00000589582.5, transcript_ENST00000590168.5, transcript_ENST00000590288.1, transcript_ENST00000590322.1, transcript_ENST00000591192.1, transcript_ENST00000591255.5, transcript_ENST00000591697.5, transcript_ENST00000591860.1, transcript_ENST00000591879.1, transcript_ENST00000591885.5, transcript_ENST00000592123.5, transcript_ENST00000592378.5, and transcript_ENST00000593103.1.

In some embodiments, the RHBDF2 pre-mRNA transcript is encoded by a genetic sequence with at least about 80%, 85%, 90%, 95%, 96%, 97%, 98%, 99% or 100% sequence identity to the Ensembl reference number ENSG00000129667.12 or a complement thereof. In some embodiments, the RHBDF2 pre-mRNA transcript comprises a sequence with at least about 80%, 85%, 90%, 95%, 96%, 97%, 98%, 99% or 100% sequence identity to a RHBDF2 pre-mRNA transcript or a complement thereof described herein.

In some embodiments, the targeted portion of the RHBDF2 pre-mRNA comprises a sequence with at least 80%, 85%, 90%, 95%, 97%, or 100% sequence identity to a region comprising at least 8 contiguous nucleic acids of a sequence of premrna_ENST00000313080.8, premrna_ENST00000585701.5, premrna_ENST00000585989.5, premrna_ENST00000587640.1, premrna_ENST00000589526.5, premrna_ENST00000589582.5, premrna_ENST00000590168.5, premrna_ENST00000590288.1, premrna_ENST00000590322.1, premrna_ENST00000591192.1, premrna_ENST00000591255.5, premrna_ENST00000591697.5, premrna_ENST00000591860.1, premrna_ENST00000591879.1, premrna_ENST00000591885.5, premrna_ENST00000592123.5, premrna_ENST00000592378.5, premrna_ENST00000593103.1 or a sequence of Table 3 or complements thereof. In some embodiments, the targeted portion of the RHBDF2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 854-SEQ ID NO: 857 or complements thereof. In some embodiments, the targeted portion of the RHBDF2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a RMND1 genomic sequence. In some embodiments, the ASO targets a NSAE pre-mRNA transcript from a RMND1 genomic sequence comprising a NSAE exon. In some embodiments, the ASO targets a NSAE pre-mRNA transcript from a RMND1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000336451.8, premrna_ENST00000444024.3, premrna_ENST00000491268.2, premrna_ENST00000622845.5, premrna_ENST00000643550.1, premrna_ENST00000643564.1, premrna_ENST00000644054.1, premrna_ENST00000644711.1, premrna_ENST00000645367.1, premrna_ENST00000645895.1, premrna_ENST00000645917.1, and premrna_ENST00000646926.1. In some embodiments, the ASO targets a RMND1 pre-mRNA sequence comprising a NSAE exon. In some embodiments, the ASO targets a RMND1 pre-mRNA sequence comprising an intron flanking the 3′ splice site of the NSAE exon. In some embodiments, the ASO targets a RMND1 pre-mRNA sequence comprising an intron flanking the 5′ splice site of the NSAE exon. In some embodiments, the transcript is selected from the group consisting of transcript_ENST00000336451.8, transcript_ENST00000444024.3, transcript_ENST00000491268.2, transcript_ENST00000622845.5, transcript_ENST00000643550.1, transcript_ENST00000643564.1, transcript_ENST00000644054.1, transcript_ENST00000644711.1, transcript_ENST00000645367.1, transcript_ENST00000645895.1, transcript_ENST00000645917.1, and transcript_ENST00000646926.1.

In some embodiments, the RMND1 pre-mRNA transcript is encoded by a genetic sequence with at least about 80%, 85%, 90%, 95%, 96%, 97%, 98%, 99% or 100% sequence identity to the Ensembl reference number ENSG00000155906.19 or a complement thereof. In some embodiments, the RMND1 pre-mRNA transcript comprises a sequence with at least about 80%, 85%, 90%, 95%, 96%, 97%, 98%, 99% or 100% sequence identity to a RMND1 pre-mRNA transcript or a complement thereof described herein.

In some embodiments, the targeted portion of the RMND1 pre-mRNA comprises a sequence with at least 80%, 85%, 90%, 95%, 97%, or 100% sequence identity to a region comprising at least 8 contiguous nucleic acids of a sequence of premrna_ENST00000336451.8, premrna_ENST00000444024.3, premrna_ENST00000491268.2, premrna_ENST00000622845.5, premrna_ENST00000643550.1, premrna_ENST00000643564.1, premrna_ENST00000644054.1, premrna_ENST00000644711.1, premrna_ENST00000645367.1, premrna_ENST00000645895.1, premrna_ENST00000645917.1, premrna_ENST00000646926.1 or a sequence of Table 3 or complements thereof. In some embodiments, the targeted portion of the RMND1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 858-SEQ ID NO: 861 or complements thereof. In some embodiments, the targeted portion of the RMND1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ROBO3 genomic sequence. In some embodiments, the ASO targets a NSAE pre-mRNA transcript from a ROBO3 genomic sequence comprising a NSAE exon. In some embodiments, the ASO targets a NSAE pre-mRNA transcript from a ROBO3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000397801.6, premrna_ENST00000524971.1, premrna_ENST00000525304.5, premrna_ENST00000525448.5, premrna_ENST00000525482.5, premrna_ENST00000526551.5, premrna_ENST00000527196.5, premrna_ENST00000527245.5, premrna_ENST00000528068.5, premrna_ENST00000528144.5, premrna_ENST00000528820.5, premrna_ENST00000529658.5, premrna_ENST00000530647.5, premrna_ENST00000531075.5, premrna_ENST00000531119.1, premrna_ENST00000531545.5, premrna_ENST00000531888.1, premrna_ENST00000532472.1, premrna_ENST00000534598.5, premrna_ENST00000538940.5, and premrna_ENST00000543966.5.

In some embodiments, the ASO targets a ROBO3 pre-mRNA sequence comprising a NSAE exon. In some embodiments, the ASO targets a ROBO3 pre-mRNA sequence comprising an intron flanking the 3′ splice site of the NSAE exon. In some embodiments, the ASO targets a ROBO3 pre-mRNA sequence comprising an intron flanking the 5′ splice site of the NSAE exon. In some embodiments, the transcript is selected from the group consisting of transcript_ENST00000397801.6, transcript_ENST00000524971.1, transcript_ENST00000525304.5, transcript_ENST00000525448.5, transcript_ENST00000525482.5, transcript_ENST00000526551.5, transcript_ENST00000527196.5, transcript_ENST00000527245.5, transcript_ENST00000528068.5, transcript_ENST00000528144.5, transcript_ENST00000528820.5, transcript_ENST00000529658.5, transcript_ENST00000530647.5, transcript_ENST00000531075.5, transcript_ENST00000531119.1, transcript_ENST00000531545.5, transcript_ENST00000531888.1, transcript_ENST00000532472.1, transcript_ENST00000534598.5, transcript_ENST00000538940.5, and transcript_ENST00000543966.5.

In some embodiments, the ROBO3 pre-mRNA transcript is encoded by a genetic sequence with at least about 80%, 85%, 90%, 95%, 96%, 97%, 98%, 99% or 100% sequence identity to the Ensembl reference number ENSG00000154134.15 or a complement thereof. In some embodiments, the ROBO3 pre-mRNA transcript comprises a sequence with at least about 80%, 85%, 90%, 95%, 96%, 97%, 98%, 99% or 100% sequence identity to a ROBO3 pre-mRNA transcript or a complement thereof described herein.

In some embodiments, the targeted portion of the ROBO3 pre-mRNA comprises a sequence with at least 80%, 85%, 90%, 95%, 97%, or 100% sequence identity to a region comprising at least 8 contiguous nucleic acids of a sequence of premrna_ENST00000397801.6, premrna_ENST00000524971.1, premrna_ENST00000525304.5, premrna_ENST00000525448.5, premrna_ENST00000525482.5, premrna_ENST00000526551.5, premrna_ENST00000527196.5, premrna_ENST00000527245.5, premrna_ENST00000528068.5, premrna_ENST00000528144.5, premrna_ENST00000528820.5, premrna_ENST00000529658.5, premrna_ENST00000530647.5, premrna_ENST00000531075.5, premrna_ENST00000531119.1, premrna_ENST00000531545.5, premrna_ENST00000531888.1, premrna_ENST00000532472.1, premrna_ENST00000534598.5, premrna_ENST00000538940.5, premrna_ENST00000543966.5, or a sequence of Table 3 or complements thereof. In some embodiments, the targeted portion of the ROBO3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 862-SEQ ID NO: 871 or complements thereof. In some embodiments, the targeted portion of the ROBO3 pre-mRNA pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a RPGRIP1 genomic sequence. In some embodiments, the ASO targets a NSAE pre-mRNA transcript from a RPGRIP1 genomic sequence comprising a NSAE exon. In some embodiments, the ASO targets a NSAE pre-mRNA transcript from a RPGRIP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000382933.8, premrna_ENST00000400017.6, premrna_ENST00000553500.5, premrna_ENST00000553927.1, premrna_ENST00000554303.1, premrna_ENST00000554750.1, premrna_ENST00000555322.5, premrna_ENST00000555489.5, premrna_ENST00000555587.5, premrna_ENST00000556336.5, premrna_ENST00000557351.1, premrna_ENST00000557606.1, and premrna_ENST00000557771.5. In some embodiments, the ASO targets a RPGRIP1 pre-mRNA sequence comprising a NSAE exon. In some embodiments, the ASO targets a RPGRIP1 pre-mRNA sequence comprising an intron flanking the 3′ splice site of the NSAE exon. In some embodiments, the ASO targets a RPGRIP1 pre-mRNA sequence comprising an intron flanking the 5′ splice site of the NSAE exon. In some embodiments, the transcript is selected from the group consisting of transcript_ENST00000382933.8, transcript_ENST00000400017.6, transcript_ENST00000553500.5, transcript_ENST00000553927.1, transcript_ENST00000554303.1, transcript_ENST00000554750.1, transcript_ENST00000555322.5, transcript_ENST00000555489.5, transcript_ENST00000555587.5, transcript_ENST00000556336.5, transcript_ENST00000557351.1, transcript_ENST00000557606.1, and transcript_ENST00000557771.5.

In some embodiments, the RPGRIP1 pre-mRNA transcript is encoded by a genetic sequence with at least about 80%, 85%, 90%, 95%, 96%, 97%, 98%, 99% or 100% sequence identity to the Ensembl reference number ENSG00000092200.12 or a complement thereof. In some embodiments, the RPGRIP1 pre-mRNA transcript comprises a sequence with at least about 80%, 85%, 90%, 95%, 96%, 97%, 98%, 99% or 100% sequence identity to a RPGRIP1 pre-mRNA transcript or a complement thereof described herein.

In some embodiments, the targeted portion of the RPGRIP1 pre-mRNA comprises a sequence with at least 80%, 85%, 90%, 95%, 97%, or 100% sequence identity to a region comprising at least 8 contiguous nucleic acids of a sequence of premrna_ENST00000382933.8, premrna_ENST00000400017.6, premrna_ENST00000553500.5, premrna_ENST00000553927.1, premrna_ENST00000554303.1, premrna_ENST00000554750.1, premrna_ENST00000555322.5, premrna_ENST00000555489.5, premrna_ENST00000555587.5, premrna_ENST00000556336.5, premrna_ENST00000557351.1, premrna_ENST00000557606.1, premrna_ENST00000557771.5 or a sequence of Table 3 or complements thereof. In some embodiments, the targeted portion of the RPGRIP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 872-SEQ ID NO: 879 or complements thereof. In some embodiments, the targeted portion of the RPGRIP1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a RTTN genomic sequence. In some embodiments, the ASO targets a NSAE pre-mRNA transcript from a RTTN genomic sequence comprising a NSAE exon. In some embodiments, the ASO targets a NSAE pre-mRNA transcript from a RTTN genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000255674.11, premrna_ENST00000578780.2, premrna_ENST00000579021.1, premrna_ENST00000579986.6, premrna_ENST00000580034.2, premrna_ENST00000581161.5, premrna_ENST00000581583.1, premrna_ENST00000581709.1, premrna_ENST00000583043.5, premrna_ENST00000583765.1, premrna_ENST00000638251.1, premrna_ENST00000638298.1, premrna_ENST00000638799.1, premrna_ENST00000639128.1, premrna_ENST00000639487.1, premrna_ENST00000640376.1, premrna_ENST00000640393.1, premrna_ENST00000640408.1, premrna_ENST00000640525.1, premrna_ENST00000640654.1, premrna_ENST00000640736.1, premrna_ENST00000640769.2, and premrna_ENST00000640931.1.

In some embodiments, the ASO targets a RTTN pre-mRNA sequence comprising a NSAE exon. In some embodiments, the ASO targets a RTTN pre-mRNA sequence comprising an intron flanking the 3′ splice site of the NSAE exon. In some embodiments, the ASO targets a RTTN pre-mRNA sequence comprising an intron flanking the 5′ splice site of the NSAE exon. In some embodiments, the transcript is selected from the group consisting of transcript_ENST00000255674.11, transcript_ENST00000578780.2, transcript_ENST00000579021.1, transcript_ENST00000579986.6, transcript_ENST00000580034.2, transcript_ENST00000581161.5, transcript_ENST00000581583.1, transcript_ENST00000581709.1, transcript_ENST00000583043.5, transcript_ENST00000583765.1, transcript_ENST00000638251.1, transcript_ENST00000638298.1, transcript_ENST00000638799.1, transcript_ENST00000639128.1, transcript_ENST00000639487.1, transcript_ENST00000640376.1, transcript_ENST00000640393.1, transcript_ENST00000640408.1, transcript_ENST00000640525.1, transcript_ENST00000640654.1, transcript_ENST00000640736.1, transcript_ENST00000640769.2, and transcript_ENST00000640931.1.

In some embodiments, the RTTN pre-mRNA transcript is encoded by a genetic sequence with at least about 80%, 85%, 90%, 95%, 96%, 97%, 98%, 99% or 100% sequence identity to the Ensembl reference number ENSG00000176225.14 or a complement thereof. In some embodiments, the RTTN pre-mRNA transcript comprises a sequence with at least about 80%, 85%, 90%, 95%, 96%, 97%, 98%, 99% or 100% sequence identity to a RTTN pre-mRNA transcript or a complement thereof described herein.

In some embodiments, the targeted portion of the RTTN pre-mRNA comprises a sequence with at least 80%, 85%, 90%, 95%, 97%, or 100% sequence identity to a region comprising at least 8 contiguous nucleic acids of a sequence of premrna_ENST00000255674.11, premrna_ENST00000578780.2, premrna_ENST00000579021.1, premrna_ENST00000579986.6, premrna_ENST00000580034.2, premrna_ENST00000581161.5, premrna_ENST00000581583.1, premrna_ENST00000581709.1, premrna_ENST00000583043.5, premrna_ENST00000583765.1, premrna_ENST00000638251.1, premrna_ENST00000638298.1, premrna_ENST00000638799.1, premrna_ENST00000639128.1, premrna_ENST00000639487.1, premrna_ENST00000640376.1, premrna_ENST00000640393.1, premrna_ENST00000640408.1, premrna_ENST00000640525.1, premrna_ENST00000640654.1, premrna_ENST00000640736.1, premrna_ENST00000640769.2, premrna_ENST00000640931.1, or a sequence of Table 3 or complements thereof. In some embodiments, the targeted portion of the RTTN pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 880-SEQ ID NO: 883 or complements thereof. In some embodiments, the targeted portion of the RTTN pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a RUFY3 genomic sequence. In some embodiments, the ASO targets a NSAE pre-mRNA transcript from a RUFY3 genomic sequence comprising a NSAE exon. In some embodiments, the ASO targets a NSAE pre-mRNA transcript from a RUFY3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000226328.8, premrna_ENST00000381006.8, premrna_ENST00000417478.6, premrna_ENST00000502653.5, premrna_ENST00000503025.5, premrna_ENST00000503876.5, premrna_ENST00000504805.6, premrna_ENST00000507333.5, premrna_ENST00000512103.5, premrna_ENST00000512331.5, premrna_ENST00000513593.1, premrna_ENST00000513597.5, premrna_ENST00000514898.1, premrna_ENST00000515442.1, and premrna_ENST00000515479.5. In some embodiments, the ASO targets a RUFY3 pre-mRNA sequence comprising a NSAE exon. In some embodiments, the ASO targets a RUFY3 pre-mRNA sequence comprising an intron flanking the 3′ splice site of the NSAE exon. In some embodiments, the ASO targets a RUFY3 pre-mRNA sequence comprising an intron flanking the 5′ splice site of the NSAE exon. In some embodiments, the transcript is selected from the group consisting of transcript_ENST00000226328.8, transcript_ENST00000381006.8, transcript_ENST00000417478.6, transcript_ENST00000502653.5, transcript_ENST00000503025.5, transcript_ENST00000503876.5, transcript_ENST00000504805.6, transcript_ENST00000507333.5, transcript_ENST00000512103.5, transcript_ENST00000512331.5, transcript_ENST00000513593.1, transcript_ENST00000513597.5, transcript_ENST00000514898.1, transcript_ENST00000515442.1, and transcript_ENST00000515479.5.

In some embodiments, the RUFY3 pre-mRNA transcript is encoded by a genetic sequence with at least about 80%, 85%, 90%, 95%, 96%, 97%, 98%, 99% or 100% sequence identity to the Ensembl reference number ENSG00000018189.13 or a complement thereof. In some embodiments, the RUFY3 pre-mRNA transcript comprises a sequence with at least about 80%, 85%, 90%, 95%, 96%, 97%, 98%, 99% or 100% sequence identity to a RUFY3 pre-mRNA transcript or a complement thereof described herein.

In some embodiments, the targeted portion of the RUFY3 pre-mRNA comprises a sequence with at least 80%, 85%, 90%, 95%, 97%, or 100% sequence identity to a region comprising at least 8 contiguous nucleic acids of a sequence of premrna_ENST00000226328.8, premrna_ENST00000381006.8, premrna_ENST00000417478.6, premrna_ENST00000502653.5, premrna_ENST00000503025.5, premrna_ENST00000503876.5, premrna_ENST00000504805.6, premrna_ENST00000507333.5, premrna_ENST00000512103.5, premrna_ENST00000512331.5, premrna_ENST00000513593.1, premrna_ENST00000513597.5, premrna_ENST00000514898.1, premrna_ENST00000515442.1, premrna_ENST00000515479.5 or a sequence of Table 3 or complements thereof. In some embodiments, the targeted portion of the RUFY3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 884-SEQ ID NO: 887 or complements thereof. In some embodiments, the targeted portion of the RUFY3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SEMA3B genomic sequence. In some embodiments, the ASO targets a NSAE pre-mRNA transcript from a SEMA3B genomic sequence comprising a NSAE exon. In some embodiments, the ASO targets a NSAE pre-mRNA transcript from a SEMA3B genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000416295.1, premrna_ENST00000418576.3, premrna_ENST00000419007.5, premrna_ENST00000433753.4, premrna_ENST00000434030.1, premrna_ENST00000439487.5, premrna_ENST00000441915.5, premrna_ENST00000456210.5, premrna_ENST00000456560.6, premrna_ENST00000611067.4, premrna_ENST00000612509.4, premrna_ENST00000616701.5, premrna_ENST00000618865.4, premrna_ENST00000619119.4, and premrna_ENST00000621029.4. In some embodiments, the ASO targets a SEMA3B pre-mRNA sequence comprising a NSAE exon. In some embodiments, the ASO targets a SEMA3B pre-mRNA sequence comprising an intron flanking the 3′ splice site of the NSAE exon. In some embodiments, the ASO targets a SEMA3B pre-mRNA sequence comprising an intron flanking the 5′ splice site of the NSAE exon. In some embodiments, the transcript is selected from the group consisting of transcript_ENST00000416295.1, transcript_ENST00000418576.3, transcript_ENST00000419007.5, transcript_ENST00000433753.4, transcript_ENST00000434030.1, transcript_ENST00000439487.5, transcript_ENST00000441915.5, transcript_ENST00000456210.5, transcript_ENST00000456560.6, transcript_ENST00000611067.4, transcript_ENST00000612509.4, transcript_ENST00000616701.5, transcript_ENST00000618865.4, transcript_ENST00000619119.4, and transcript_ENST00000621029.4.

In some embodiments, the SEMA3B pre-mRNA transcript is encoded by a genetic sequence with at least about 80%, 85%, 90%, 95%, 96%, 97%, 98%, 99% or 100% sequence identity to the Ensembl reference number ENSG00000012171.20 or ENSG00000232352.1 or a complement thereof. In some embodiments, the SEMA3B pre-mRNA transcript comprises a sequence with at least about 80%, 85%, 90%, 95%, 96%, 97%, 98%, 99% or 100% sequence identity to a SEMA3B pre-mRNA transcript or a complement thereof described herein.

In some embodiments, the targeted portion of the SEMA3B pre-mRNA comprises a sequence with at least 80%, 85%, 90%, 95%, 97%, or 100% sequence identity to a region comprising at least 8 contiguous nucleic acids of a sequence of premrna_ENST00000416295.1, premrna_ENST00000418576.3, premrna_ENST00000419007.5, premrna_ENST00000433753.4, premrna_ENST00000434030.1, premrna_ENST00000439487.5, premrna_ENST00000441915.5, premrna_ENST00000456210.5, premrna_ENST00000456560.6, premrna_ENST00000611067.4, premrna_ENST00000612509.4, premrna_ENST00000616701.5, premrna_ENST00000618865.4, premrna_ENST00000619119.4, premrna_ENST00000621029.4 or a sequence of Table 3 or complements thereof. In some embodiments, the targeted portion of the SEMA3B pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 888-SEQ ID NO: 891 or complements thereof. In some embodiments, the targeted portion of the SEMA3B pre-mRNA pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SEMA3F genomic sequence. In some embodiments, the ASO targets a NSAE pre-mRNA transcript from a SEMA3F genomic sequence comprising a NSAE exon. In some embodiments, the ASO targets a NSAE pre-mRNA transcript from a SEMA3F genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000002829.8, premrna_ENST00000413852.5, premrna_ENST00000414301.5, premrna_ENST00000420831.1, premrna_ENST00000426511.5, premrna_ENST00000434342.5, premrna_ENST00000450338.5, premrna_ENST00000470737.1, and premrna_ENST00000493743.1. In some embodiments, the ASO targets a SEMA3F pre-mRNA sequence comprising a NSAE exon. In some embodiments, the ASO targets a SEMA3F pre-mRNA sequence comprising an intron flanking the 3′ splice site of the NSAE exon. In some embodiments, the ASO targets a SEMA3F pre-mRNA sequence comprising an intron flanking the 5′ splice site of the NSAE exon. In some embodiments, the transcript is selected from the group consisting of transcript_ENST00000002829.8, transcript_ENST00000413852.5, transcript_ENST00000414301.5, transcript_ENST00000420831.1, transcript_ENST00000426511.5, transcript_ENST00000434342.5, transcript_ENST00000450338.5, transcript_ENST00000470737.1, and transcript_ENST00000493743.1.

In some embodiments, the SEMA3F pre-mRNA transcript is encoded by a genetic sequence with at least about 80%, 85%, 90%, 95%, 96%, 97%, 98%, 99% or 100% sequence identity to the Ensembl reference number ENSG00000001617.12 or ENSG00000235016.1 or a complement thereof. In some embodiments, the SEMA3F pre-mRNA transcript comprises a sequence with at least about 80%, 85%, 90%, 95%, 96%, 97%, 98%, 99% or 100% sequence identity to a SEMA3F pre-mRNA transcript or a complement thereof described herein.

In some embodiments, the targeted portion of the SEMA3F pre-mRNA comprises a sequence with at least 80%, 85%, 90%, 95%, 97%, or 100% sequence identity to a region comprising at least 8 contiguous nucleic acids of a sequence of premrna_ENST00000002829.8, premrna_ENST00000413852.5, premrna_ENST00000414301.5, premrna_ENST00000420831.1, premrna_ENST00000426511.5, premrna_ENST00000434342.5, premrna_ENST00000450338.5, premrna_ENST00000470737.1, premrna_ENST00000493743.1 or a sequence of Table 3 or complements thereof. In some embodiments, the targeted portion of the SEMA3F pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 892-SEQ ID NO: 895 or complements thereof. In some embodiments, the targeted portion of the SEMA3F pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SH2D3A genomic sequence. In some embodiments, the ASO targets a NSAE pre-mRNA transcript from a SH2D3A genomic sequence comprising a NSAE exon. In some embodiments, the ASO targets a NSAE pre-mRNA transcript from a SH2D3A genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000245908.11, premrna_ENST00000437152.7, premrna_ENST00000595369.1, premrna_ENST00000595681.5, premrna_ENST00000597168.1, premrna_ENST00000597254.1, premrna_ENST00000597687.1, and premrna_ENST00000599563.5. In some embodiments, the ASO targets a SH2D3A pre-mRNA sequence comprising a NSAE exon. In some embodiments, the ASO targets a SH2D3A pre-mRNA sequence comprising an intron flanking the 3′ splice site of the NSAE exon. In some embodiments, the ASO targets a SH2D3A pre-mRNA sequence comprising an intron flanking the 5′ splice site of the NSAE exon. In some embodiments, the transcript is selected from the group consisting of transcript_ENST00000245908.11, transcript_ENST00000437152.7, transcript_ENST00000595369.1, transcript_ENST00000595681.5, transcript_ENST00000597168.1, transcript_ENST00000597254.1, transcript_ENST00000597687.1, and transcript_ENST00000599563.5.

In some embodiments, the SH2D3A pre-mRNA transcript is encoded by a genetic sequence with at least about 80%, 85%, 90%, 95%, 96%, 97%, 98%, 99% or 100% sequence identity to the Ensembl reference number ENSG00000125731.13 or a complement thereof. In some embodiments, the SH2D3A pre-mRNA transcript comprises a sequence with at least about 80%, 85%, 90%, 95%, 96%, 97%, 98%, 99% or 100% sequence identity to a SH2D3A pre-mRNA transcript or a complement thereof described herein.

In some embodiments, the targeted portion of the SH2D3A pre-mRNA comprises a sequence with at least 80%, 85%, 90%, 95%, 97%, or 100% sequence identity to a region comprising at least 8 contiguous nucleic acids of a sequence of premrna_ENST00000245908.11, premrna_ENST00000437152.7, premrna_ENST00000595369.1, premrna_ENST00000595681.5, premrna_ENST00000597168.1, premrna_ENST00000597254.1, premrna_ENST00000597687.1, premrna_ENST00000599563.5 or a sequence of Table 3 or complements thereof. In some embodiments, the targeted portion of the SH2D3A pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 896-SEQ ID NO: 901 or complements thereof. In some embodiments, the targeted portion of the SH2D3A pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SIK3 genomic sequence. In some embodiments, the ASO targets a NSAE pre-mRNA transcript from a SIK3 genomic sequence comprising a NSAE exon. In some embodiments, the ASO targets a NSAE pre-mRNA transcript from a SIK3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000375300.6, premrna_ENST00000413553.1, premrna_ENST00000415541.5, premrna_ENST00000445177.5, premrna_ENST00000446921.6, premrna_ENST00000454905.5, premrna_ENST00000465421.5, premrna_ENST00000472648.1, premrna_ENST00000480222.1, premrna_ENST00000480468.1, premrna_ENST00000485363.1, premrna_ENST00000488337.5, and premrna_ENST00000497049.5. In some embodiments, the ASO targets a SIK3 pre-mRNA sequence comprising a NSAE exon. In some embodiments, the ASO targets a SIK3 pre-mRNA sequence comprising an intron flanking the 3′ splice site of the NSAE exon. In some embodiments, the ASO targets a SIK3 pre-mRNA sequence comprising an intron flanking the 5′ splice site of the NSAE exon. In some embodiments, the transcript is selected from the group consisting of transcript_ENST00000375300.6, transcript_ENST00000413553.1, transcript_ENST00000415541.5, transcript_ENST00000445177.5, transcript_ENST00000446921.6, transcript_ENST00000454905.5, transcript_ENST00000465421.5, transcript_ENST00000472648.1, transcript_ENST00000480222.1, transcript_ENST00000480468.1, transcript_ENST00000485363.1, transcript_ENST00000488337.5, and transcript_ENST00000497049.5.

In some embodiments, the SIK3 pre-mRNA transcript is encoded by a genetic sequence with at least about 80%, 85%, 90%, 95%, 96%, 97%, 98%, 99% or 100% sequence identity to the Ensembl reference number ENSG00000160584.16 or a complement thereof. In some embodiments, the SIK3 pre-mRNA transcript comprises a sequence with at least about 80%, 85%, 90%, 95%, 96%, 97%, 98%, 99% or 100% sequence identity to a SIK3 pre-mRNA transcript or a complement thereof described herein.

In some embodiments, the targeted portion of the SIK3 pre-mRNA comprises a sequence with at least 80%, 85%, 90%, 95%, 97%, or 100% sequence identity to a region comprising at least 8 contiguous nucleic acids of a sequence of premrna_ENST00000375300.6, premrna_ENST00000413553.1, premrna_ENST00000415541.5, premrna_ENST00000445177.5, premrna_ENST00000446921.6, premrna_ENST00000454905.5, premrna_ENST00000465421.5, premrna_ENST00000472648.1, premrna_ENST00000480222.1, premrna_ENST00000480468.1, premrna_ENST00000485363.1, premrna_ENST00000488337.5, premrna_ENST00000497049.5, or a sequence of Table 3 or complements thereof. In some embodiments, the targeted portion of the SIK3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 902-SEQ ID NO: 905 or complements thereof. In some embodiments, the targeted portion of the SIK3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SIRT3 genomic sequence. In some embodiments, the ASO targets a NSAE pre-mRNA transcript from a SIRT3 genomic sequence comprising a NSAE exon. In some embodiments, the ASO targets a NSAE pre-mRNA transcript from a SIRT3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000382743.9, premrna_ENST00000524564.5, premrna_ENST00000525237.1, premrna_ENST00000525319.5, premrna_ENST00000525776.1, premrna_ENST00000526854.5, premrna_ENST00000528469.1, premrna_ENST00000528702.5, premrna_ENST00000529055.5, premrna_ENST00000529382.5, premrna_ENST00000529937.1, premrna_ENST00000530067.1, premrna_ENST00000531753.5, premrna_ENST00000532837.5, premrna_ENST00000532956.5, and premrna_ENST00000534381.1. In some embodiments, the ASO targets a SIRT3 pre-mRNA sequence comprising a NSAE exon. In some embodiments, the ASO targets a SIRT3 pre-mRNA sequence comprising an intron flanking the 3′ splice site of the NSAE exon. In some embodiments, the ASO targets a SIRT3 pre-mRNA sequence comprising an intron flanking the 5′ splice site of the NSAE exon. In some embodiments, the transcript is selected from the group consisting of transcript_ENST00000382743.9, transcript_ENST00000524564.5, transcript_ENST00000525237.1, transcript_ENST00000525319.5, transcript_ENST00000525776.1, transcript_ENST00000526854.5, transcript_ENST00000528469.1, transcript_ENST00000528702.5, transcript_ENST00000529055.5, transcript_ENST00000529382.5, transcript_ENST00000529937.1, transcript_ENST00000530067.1, transcript_ENST00000531753.5, transcript_ENST00000532837.5, transcript_ENST00000532956.5, and transcript_ENST00000534381.1.

In some embodiments, the SIRT3 pre-mRNA transcript is encoded by a genetic sequence with at least about 80%, 85%, 90%, 95%, 96%, 97%, 98%, 99% or 100% sequence identity to the Ensembl reference number ENSG00000142082.15 or a complement thereof. In some embodiments, the SIRT3 pre-mRNA transcript comprises a sequence with at least about 80%, 85%, 90%, 95%, 96%, 97%, 98%, 99% or 100% sequence identity to a SIRT3 pre-mRNA transcript or a complement thereof described herein.

In some embodiments, the targeted portion of the SIRT3 pre-mRNA comprises a sequence with at least 80%, 85%, 90%, 95%, 97%, or 100% sequence identity to a region comprising at least 8 contiguous nucleic acids of a sequence of premrna_ENST00000382743.9, premrna_ENST00000524564.5, premrna_ENST00000525237.1, premrna_ENST00000525319.5, premrna_ENST00000525776.1, premrna_ENST00000526854.5, premrna_ENST00000528469.1, premrna_ENST00000528702.5, premrna_ENST00000529055.5, premrna_ENST00000529382.5, premrna_ENST00000529937.1, premrna_ENST00000530067.1, premrna_ENST00000531753.5, premrna_ENST00000532837.5, premrna_ENST00000532956.5, premrna_ENST00000534381.1 or a sequence of Table 3 or complements thereof. In some embodiments, the targeted portion of the SIRT3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 906-SEQ ID NO: 909 or complements thereof. In some embodiments, the targeted portion of the SIRT3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SKIV2L genomic sequence. In some embodiments, the ASO targets a NSAE pre-mRNA transcript from a SKIV2L genomic sequence comprising a NSAE exon. In some embodiments, the ASO targets a NSAE pre-mRNA transcript from a SKIV2L genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000375394.7, premrna_ENST00000461073.5, premrna_ENST00000461915.5, premrna_ENST00000465703.5, premrna_ENST00000466290.1, premrna_ENST00000470453.1, premrna_ENST00000471818.1, premrna_ENST00000474839.5, premrna_ENST00000483553.5, premrna_ENST00000484835.1, premrna_ENST00000485349.5, premrna_ENST00000488648.5, premrna_ENST00000491994.1, premrna_ENST00000492900.1, premma_ENST00000494058.5, and premrna_ENST00000628157.1. In some embodiments, the ASO targets a SKIV2L pre-mRNA sequence comprising a NSAE exon. In some embodiments, the ASO targets a SKIV2L pre-mRNA sequence comprising an intron flanking the 3′ splice site of the NSAE exon. In some embodiments, the ASO targets a SKIV2L pre-mRNA sequence comprising an intron flanking the 5′ splice site of the NSAE exon. In some embodiments, the transcript is selected from the group consisting of transcript_ENST00000375394.7, transcript_ENST00000461073.5, transcript_ENST00000461915.5, transcript_ENST00000465703.5, transcript_ENST00000466290.1, transcript_ENST00000470453.1, transcript_ENST00000471818.1, transcript_ENST00000474839.5, transcript_ENST00000483553.5, transcript_ENST00000484835.1, transcript_ENST00000485349.5, transcript_ENST00000488648.5, transcript_ENST00000491994.1, transcript_ENST00000492900.1, transcript_ENST00000494058.5, and transcript_ENST00000628157.1.

In some embodiments, the SKIV2L pre-mRNA transcript is encoded by a genetic sequence with at least about 80%, 85%, 90%, 95%, 96%, 97%, 98%, 99% or 100% sequence identity to the Ensembl reference number ENSG00000204351.12, ENSG00000206353.11, ENSG00000228896.9, ENSG00000232616.9, ENSG00000223493.9, or ENSG00000225737.9 or a complement thereof. In some embodiments, the SKIV2L pre-mRNA transcript comprises a sequence with at least about 80%, 85%, 90%, 95%, 96%, 97%, 98%, 99% or 100% sequence identity to a SKIV2L pre-mRNA transcript or a complement thereof described herein.

In some embodiments, the targeted portion of the SKIV2L pre-mRNA comprises a sequence with at least 80%, 85%, 90%, 95%, 97%, or 100% sequence identity to a region comprising at least 8 contiguous nucleic acids of a sequence of premrna_ENST00000375394.7, premrna_ENST00000461073.5, premrna_ENST00000461915.5, premrna_ENST00000465703.5, premrna_ENST00000466290.1, premrna_ENST00000470453.1, premrna_ENST00000471818.1, premrna_ENST00000474839.5, premrna_ENST00000483553.5, premrna_ENST00000484835.1, premrna_ENST00000485349.5, premrna_ENST00000488648.5, premrna_ENST00000491994.1, premrna_ENST00000492900.1, premrna_ENST00000494058.5, premrna_ENST00000628157.1 or a sequence of Table 3 or complements thereof. In some embodiments, the targeted portion of the SKIV2L pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 910-SEQ ID NO: 913 or complements thereof. In some embodiments, the targeted portion of the SKIV2L pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC2A13 genomic sequence. In some embodiments, the ASO targets a NSAE pre-mRNA transcript from a SLC2A13 genomic sequence comprising a NSAE exon. In some embodiments, the ASO targets a NSAE pre-mRNA transcript from a SLC2A13 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000280871.9, premrna_ENST00000380858.1, premrna_ENST00000465517.1, and premrna_ENST00000505338.1. In some embodiments, the ASO targets a SLC2A13 pre-mRNA sequence comprising a NSAE exon. In some embodiments, the ASO targets a SLC2A13 pre-mRNA sequence comprising an intron flanking the 3′ splice site of the NSAE exon. In some embodiments, the ASO targets a SLC2A13 pre-mRNA sequence comprising an intron flanking the 5′ splice site of the NSAE exon. In some embodiments, the transcript is selected from the group consisting of transcript_ENST00000280871.9, transcript_ENST00000380858.1, transcript_ENST00000465517.1, and transcript_ENST00000505338.1.

In some embodiments, the SLC2A13 pre-mRNA transcript is encoded by a genetic sequence with at least about 80%, 85%, 90%, 95%, 96%, 97%, 98%, 99% or 100% sequence identity to the Ensembl reference number ENSG00000151229.13 or a complement thereof. In some embodiments, the SLC2A13 pre-mRNA transcript comprises a sequence with at least about 80%, 85%, 90%, 95%, 96%, 97%, 98%, 99% or 100% sequence identity to a SLC2A13 pre-mRNA transcript or a complement thereof described herein.

In some embodiments, the targeted portion of the SLC2A13 pre-mRNA comprises a sequence with at least 80%, 85%, 90%, 95%, 97%, or 100% sequence identity to a region comprising at least 8 contiguous nucleic acids of a sequence of premrna_ENST00000280871.9, premrna_ENST00000380858.1, premrna_ENST00000465517.1, premrna_ENST00000505338.1 or a sequence of Table 3 or complements thereof. In some embodiments, the targeted portion of the SLC2A13 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 914-SEQ ID NO: 917 or complements thereof. In some embodiments, the targeted portion of the SLC2A13 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC12A7 genomic sequence. In some embodiments, the ASO targets a NSAE pre-mRNA transcript from a SLC12A7 genomic sequence comprising a NSAE exon. In some embodiments, the ASO targets a NSAE pre-mRNA transcript from a SLC12A7 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000264930.10, premrna_ENST00000504576.2, premrna_ENST00000510943.3, premrna_ENST00000513223.2, premrna_ENST00000514994.1, and premrna_ENST00000634447.1. In some embodiments, the ASO targets a SLC12A7 pre-mRNA sequence comprising a NSAE exon. In some embodiments, the ASO targets a SLC12A7 pre-mRNA sequence comprising an intron flanking the 3′ splice site of the NSAE exon. In some embodiments, the ASO targets a SLC12A7 pre-mRNA sequence comprising an intron flanking the 5′ splice site of the NSAE exon. In some embodiments, the transcript is selected from the group consisting of transcript_ENST00000264930.10, transcript_ENST00000504576.2, transcript_ENST00000510943.3, transcript_ENST00000513223.2, transcript_ENST00000514994.1, and transcript_ENST00000634447.1.

In some embodiments, the SLC12A7 pre-mRNA transcript is encoded by a genetic sequence with at least about 80%, 85%, 90%, 95%, 96%, 97%, 98%, 99% or 100% sequence identity to the Ensembl reference number ENSG00000113504.21 or ENSG00000276482.3 or a complement thereof. In some embodiments, the SLC12A7 pre-mRNA transcript comprises a sequence with at least about 80%, 85%, 90%, 95%, 96%, 97%, 98%, 99% or 100% sequence identity to a SLC12A7 pre-mRNA transcript or a complement thereof described herein.

In some embodiments, the targeted portion of the SLC12A7 pre-mRNA comprises a sequence with at least 80%, 85%, 90%, 95%, 97%, or 100% sequence identity to a region comprising at least 8 contiguous nucleic acids of a sequence of premrna_ENST00000264930.10, premrna_ENST00000504576.2, premrna_ENST00000510943.3, premrna_ENST00000513223.2, premrna_ENST00000514994.1, premrna_ENST00000634447.1, or a sequence of Table 3 or complements thereof. In some embodiments, the targeted portion of the SLC12A7 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 918-SEQ ID NO: 921 or complements thereof. In some embodiments, the targeted portion of the SLC12A7 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC22A3 genomic sequence. In some embodiments, the ASO targets a NSAE pre-mRNA transcript from a SLC22A3 genomic sequence comprising a NSAE exon. In some embodiments, the ASO targets a NSAE pre-mRNA transcript from a SLC22A3 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence of premrna_ENST00000275300.3. In some embodiments, the ASO targets a SLC22A3 pre-mRNA sequence comprising a NSAE exon. In some embodiments, the ASO targets a SLC22A3 pre-mRNA sequence comprising an intron flanking the 3′ splice site of the NSAE exon. In some embodiments, the ASO targets a SLC22A3 pre-mRNA sequence comprising an intron flanking the 5′ splice site of the NSAE exon. In some embodiments, the transcript is selected from the group consisting of transcript_ENST00000275300.3.

In some embodiments, the SLC22A3 pre-mRNA transcript is encoded by a genetic sequence with at least about 80%, 85%, 90%, 95%, 96%, 97%, 98%, 99% or 100% sequence identity to the Ensembl reference number ENSG00000146477.6 or a complement thereof. In some embodiments, the SLC22A3 pre-mRNA transcript comprises a sequence with at least about 80%, 85%, 90%, 95%, 96%, 97%, 98%, 99% or 100% sequence identity to a SLC22A3 pre-mRNA transcript or a complement thereof described herein.

In some embodiments, the targeted portion of the SLC22A3 pre-mRNA comprises a sequence with at least 80%, 85%, 90%, 95%, 97%, or 100% sequence identity to a region comprising at least 8 contiguous nucleic acids of a sequence of premrna_ENST00000275300.3 or a sequence of Table 3 or complements thereof. In some embodiments, the targeted portion of the SLC22A3 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 922-SEQ ID NO: 925 or complements thereof. In some embodiments, the targeted portion of the SLC22A3 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC25A13 genomic sequence. In some embodiments, the ASO targets a NSAE pre-mRNA transcript from a SLC25A13 genomic sequence comprising a NSAE exon. In some embodiments, the ASO targets a NSAE pre-mRNA transcript from a SLC25A13 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000265631.9, premrna_ENST00000416240.6, premrna_ENST00000472162.2, premrna_ENST00000484495.5, premrna_ENST00000487710.1, premrna_ENST00000490072.5, premrna_ENST00000492869.1, and premrna_ENST00000494085.1. In some embodiments, the ASO targets a SLC25A13 pre-mRNA sequence comprising a NSAE exon. In some embodiments, the ASO targets a SLC25A13 pre-mRNA sequence comprising an intron flanking the 3′ splice site of the NSAE exon. In some embodiments, the ASO targets a SLC25A13 pre-mRNA sequence comprising an intron flanking the 5′ splice site of the NSAE exon. In some embodiments, the transcript is selected from the group consisting of transcript_ENST00000265631.9, transcript_ENST00000416240.6, transcript_ENST00000472162.2, transcript_ENST00000484495.5, transcript_ENST00000487710.1, transcript_ENST00000490072.5, transcript_ENST00000492869.1, and transcript_ENST00000494085.1.

In some embodiments, the SLC25A13 pre-mRNA transcript is encoded by a genetic sequence with at least about 80%, 85%, 90%, 95%, 96%, 97%, 98%, 99% or 100% sequence identity to the Ensembl reference number ENSG00000004864.14 or a complement thereof. In some embodiments, the SLC25A13 pre-mRNA transcript comprises a sequence with at least about 80%, 85%, 90%, 95%, 96%, 97%, 98%, 99% or 100% sequence identity to a SLC25A13 pre-mRNA transcript or a complement thereof described herein.

In some embodiments, the targeted portion of the SLC25A13 pre-mRNA comprises a sequence with at least 80%, 85%, 90%, 95%, 97%, or 100% sequence identity to a region comprising at least 8 contiguous nucleic acids of a sequence of premrna_ENST00000265631.9, premrna_ENST00000416240.6, premrna_ENST00000472162.2, premrna_ENST00000484495.5, premrna_ENST00000487710.1, premrna_ENST00000490072.5, premrna_ENST00000492869.1, premrna_ENST00000494085.1 or a sequence of Table 3 or complements thereof. In some embodiments, the targeted portion of the SLC25A13 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 926-SEQ ID NO: 929 or complements thereof. In some embodiments, the targeted portion of the SLC25A13 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC25A37 genomic sequence. In some embodiments, the ASO targets a NSAE pre-mRNA transcript from a SLC25A37 genomic sequence comprising a NSAE exon. In some embodiments, the ASO targets a NSAE pre-mRNA transcript from a SLC25A37 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000290075.10, premrna_ENST00000417331.6, premrna_ENST00000517923.1, premrna_ENST00000518881.5, premrna_ENST00000519192.1, premrna_ENST00000519973.6, premrna_ENST00000520654.1, premrna_ENST00000520949.1, premrna_ENST00000521637.1, premrna_ENST00000522164.5, premrna_ENST00000523883.5, and premrna_ENST00000523930.1. In some embodiments, the ASO targets a SLC25A37 pre-mRNA sequence comprising a NSAE exon. In some embodiments, the ASO targets a SLC25A37 pre-mRNA sequence comprising an intron flanking the 3′ splice site of the NSAE exon. In some embodiments, the ASO targets a SLC25A37 pre-mRNA sequence comprising an intron flanking the 5′ splice site of the NSAE exon. In some embodiments, the transcript is selected from the group consisting of transcript_ENST00000290075.10, transcript_ENST00000417331.6, transcript_ENST00000517923.1, transcript_ENST00000518881.5, transcript_ENST00000519192.1, transcript_ENST00000519973.6, transcript_ENST00000520654.1, transcript_ENST00000520949.1, transcript_ENST00000521637.1, transcript_ENST00000522164.5, transcript_ENST00000523883.5, and transcript_ENST00000523930.1.

In some embodiments, the SLC25A37 pre-mRNA transcript is encoded by a genetic sequence with at least about 80%, 85%, 90%, 95%, 96%, 97%, 98%, 99% or 100% sequence identity to the Ensembl reference number ENSG00000147454.14 or a complement thereof. In some embodiments, the SLC25A37 pre-mRNA transcript comprises a sequence with at least about 80%, 85%, 90%, 95%, 96%, 97%, 98%, 99% or 100% sequence identity to a SLC25A37 pre-mRNA transcript or a complement thereof described herein.

In some embodiments, the targeted portion of the SLC25A37 pre-mRNA comprises a sequence with at least 80%, 85%, 90%, 95%, 97%, or 100% sequence identity to a region comprising at least 8 contiguous nucleic acids of a sequence of premrna_ENST00000290075.10, premrna_ENST00000417331.6, premrna_ENST00000517923.1, premrna_ENST00000518881.5, premrna_ENST00000519192.1, premrna_ENST00000519973.6, premrna_ENST00000520654.1, premrna_ENST00000520949.1, premrna_ENST00000521637.1, premrna_ENST00000522164.5, premrna_ENST00000523883.5, premrna_ENST00000523930.1 or a sequence of Table 3 or complements thereof. In some embodiments, the targeted portion of the SLC25A37 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 930-SEQ ID NO: 933 or complements thereof. In some embodiments, the targeted portion of the SLC25A37 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC27A5 genomic sequence. In some embodiments, the ASO targets a NSAE pre-mRNA transcript from a SLC27A5 genomic sequence comprising a NSAE exon. In some embodiments, the ASO targets a NSAE pre-mRNA transcript from a SLC27A5 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000263093.7, premrna_ENST00000593745.1, premrna_ENST00000594683.1, premrna_ENST00000594786.1, premrna_ENST00000595851.5, premrna_ENST00000599700.1, premrna_ENST00000601355.1, and premrna_ENST00000601997.1. In some embodiments, the ASO targets a SLC27A5 pre-mRNA sequence comprising a NSAE exon. In some embodiments, the ASO targets a SLC27A5 pre-mRNA sequence comprising an intron flanking the 3′ splice site of the NSAE exon. In some embodiments, the ASO targets a SLC27A5 pre-mRNA sequence comprising an intron flanking the 5′ splice site of the NSAE exon. In some embodiments, the transcript is selected from the group consisting of transcript_ENST00000263093.7, transcript_ENST00000593745.1, transcript_ENST00000594683.1, transcript_ENST00000594786.1, transcript_ENST00000595851.5, transcript_ENST00000599700.1, transcript_ENST00000601355.1, and transcript_ENST00000601997.1.

In some embodiments, the SLC27A5 pre-mRNA transcript is encoded by a genetic sequence with at least about 80%, 85%, 90%, 95%, 96%, 97%, 98%, 99% or 100% sequence identity to the Ensembl reference number ENSG00000083807.10 or a complement thereof. In some embodiments, the SLC27A5 pre-mRNA transcript comprises a sequence with at least about 80%, 85%, 90%, 95%, 96%, 97%, 98%, 99% or 100% sequence identity to a SLC27A5 pre-mRNA transcript or a complement thereof described herein.

In some embodiments, the targeted portion of the SLC27A5 pre-mRNA comprises a sequence with at least 80%, 85%, 90%, 95%, 97%, or 100% sequence identity to a region comprising at least 8 contiguous nucleic acids of a sequence of premrna_ENST00000263093.7, premrna_ENST00000593745.1, premrna_ENST00000594683.1, premrna_ENST00000594786.1, premrna_ENST00000595851.5, premrna_ENST00000599700.1, premrna_ENST00000601355.1, premrna_ENST00000601997.1, or a sequence of Table 3 or complements thereof. In some embodiments, the targeted portion of the SLC27A5 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 934-SEQ ID NO: 937 or complements thereof. In some embodiments, the targeted portion of the SLC27A5 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC30A9 genomic sequence. In some embodiments, the ASO targets a NSAE pre-mRNA transcript from a SLC30A9 genomic sequence comprising a NSAE exon. In some embodiments, the ASO targets a NSAE pre-mRNA transcript from a SLC30A9 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000264451.12, premrna_ENST00000505523.1, premrna_ENST00000509683.5, premrna_ENST00000510460.1, and premrna_ENST00000513699.5. In some embodiments, the ASO targets a SLC30A9 pre-mRNA sequence comprising a NSAE exon. In some embodiments, the ASO targets a SLC30A9 pre-mRNA sequence comprising an intron flanking the 3′ splice site of the NSAE exon. In some embodiments, the ASO targets a SLC30A9 pre-mRNA sequence comprising an intron flanking the 5′ splice site of the NSAE exon. In some embodiments, the transcript is selected from the group consisting of transcript_ENST00000264451.12, transcript_ENST00000505523.1, transcript_ENST00000509683.5, transcript_ENST00000510460.1, and transcript_ENST00000513699.5.

In some embodiments, the SLC30A9 pre-mRNA transcript is encoded by a genetic sequence with at least about 80%, 85%, 90%, 95%, 96%, 97%, 98%, 99% or 100% sequence identity to the Ensembl reference number ENSG00000014824.14 or a complement thereof. In some embodiments, the SLC30A9 pre-mRNA transcript comprises a sequence with at least about 80%, 85%, 90%, 95%, 96%, 97%, 98%, 99% or 100% sequence identity to a SLC30A9 pre-mRNA transcript or a complement thereof described herein.

In some embodiments, the targeted portion of the SLC30A9 pre-mRNA comprises a sequence with at least 80%, 85%, 90%, 95%, 97%, or 100% sequence identity to a region comprising at least 8 contiguous nucleic acids of a sequence of premrna_ENST00000264451.12, premrna_ENST00000505523.1, premrna_ENST00000509683.5, premrna_ENST00000510460.1, premrna_ENST00000513699.5 or a sequence of Table 3 or complements thereof. In some embodiments, the targeted portion of the SLC30A9 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 938-SEQ ID NO: 941 or complements thereof. In some embodiments, the targeted portion of the SLC30A9 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SLC30A10 genomic sequence. In some embodiments, the ASO targets a NSAE pre-mRNA transcript from a SLC30A10 genomic sequence comprising a NSAE exon. In some embodiments, the ASO targets a NSAE pre-mRNA transcript from a SLC30A10 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000356609.2, premrna_ENST00000366926.3, premrna_ENST00000484079.1, and premrna_ENST00000484239.5. In some embodiments, the ASO targets a SLC30A10 pre-mRNA sequence comprising a NSAE exon. In some embodiments, the ASO targets a SLC30A10 pre-mRNA sequence comprising an intron flanking the 3′ splice site of the NSAE exon. In some embodiments, the ASO targets a SLC30A10 pre-mRNA sequence comprising an intron flanking the 5′ splice site of the NSAE exon. In some embodiments, the transcript is selected from the group consisting of transcript_ENST00000356609.2, transcript_ENST00000366926.3, transcript_ENST00000484079.1, and transcript_ENST00000484239.5.

In some embodiments, the SLC30A10 pre-mRNA transcript is encoded by a genetic sequence with at least about 80%, 85%, 90%, 95%, 96%, 97%, 98%, 99% or 100% sequence identity to the Ensembl reference number ENSG00000196660.11 or a complement thereof. In some embodiments, the SLC30A10 pre-mRNA transcript comprises a sequence with at least about 80%, 85%, 90%, 95%, 96%, 97%, 98%, 99% or 100% sequence identity to a SLC30A10 pre-mRNA transcript or a complement thereof described herein.

In some embodiments, the targeted portion of the SLC30A10 pre-mRNA comprises a sequence with at least 80%, 85%, 90%, 95%, 97%, or 100% sequence identity to a region comprising at least 8 contiguous nucleic acids of a sequence of premrna_ENST00000356609.2, premrna_ENST00000366926.3, premrna_ENST00000484079.1, premrna_ENST00000484239.5 or a sequence of Table 3 or complements thereof. In some embodiments, the targeted portion of the SLC30A10 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 942-SEQ ID NO: 945 or complements thereof. In some embodiments, the targeted portion of the SLC30A10 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SMPD1 genomic sequence. In some embodiments, the ASO targets a NSAE pre-mRNA transcript from a SMPD1 genomic sequence comprising a NSAE exon. In some embodiments, the ASO targets a NSAE pre-mRNA transcript from a SMPD1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000342245.9, premrna_ENST00000526280.1, premrna_ENST00000527275.5, premrna_ENST00000530395.1, premrna_ENST00000531303.5, premrna_ENST00000531336.1, premrna_ENST00000532367.1, premrna_ENST00000533123.5, premrna_ENST00000533196.1, and premrna_ENST00000534405.5. In some embodiments, the ASO targets a SMPD1 pre-mRNA sequence comprising a NSAE exon. In some embodiments, the ASO targets a SMPD1 pre-mRNA sequence comprising an intron flanking the 3′ splice site of the NSAE exon. In some embodiments, the ASO targets a SMPD1 pre-mRNA sequence comprising an intron flanking the 5′ splice site of the NSAE exon. In some embodiments, the transcript is selected from the group consisting of transcript_ENST00000342245.9, transcript_ENST00000526280.1, transcript_ENST00000527275.5, transcript_ENST00000530395.1, transcript_ENST00000531303.5, transcript_ENST00000531336.1, transcript_ENST00000532367.1, transcript_ENST00000533123.5, transcript_ENST00000533196.1, and transcript_ENST00000534405.5.

In some embodiments, the SMPD1 pre-mRNA transcript is encoded by a genetic sequence with at least about 80%, 85%, 90%, 95%, 96%, 97%, 98%, 99% or 100% sequence identity to the Ensembl reference number ENSG00000166311.10 or a complement thereof. In some embodiments, the SMPD1 pre-mRNA transcript comprises a sequence with at least about 80%, 85%, 90%, 95%, 96%, 97%, 98%, 99% or 100% sequence identity to a SMPD1 pre-mRNA transcript or a complement thereof described herein.

In some embodiments, the targeted portion of the SMPD1 pre-mRNA comprises a sequence with at least 80%, 85%, 90%, 95%, 97%, or 100% sequence identity to a region comprising at least 8 contiguous nucleic acids of a sequence of premrna_ENST00000342245.9, premrna_ENST00000526280.1, premrna_ENST00000527275.5, premrna_ENST00000530395.1, premrna_ENST00000531303.5, premrna_ENST00000531336.1, premrna_ENST00000532367.1, premrna_ENST00000533123.5, premrna_ENST00000533196.1, premrna_ENST00000534405.5 or a sequence of Table 3 or complements thereof. In some embodiments, the targeted portion of the SMPD1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 946-SEQ ID NO: 951 or complements thereof. In some embodiments, the targeted portion of the SMPD1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SMTN genomic sequence. In some embodiments, the ASO targets a NSAE pre-mRNA transcript from a SMTN genomic sequence comprising a NSAE exon. In some embodiments, the ASO targets a NSAE pre-mRNA transcript from a SMTN genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000333137.11, premrna_ENST00000347557.6, premrna_ENST00000358743.5, premrna_ENST00000404574.5, premrna_ENST00000416786.5, premrna_ENST00000422839.5, premrna_ENST00000426927.5, premrna_ENST00000431481.1, premrna_ENST00000432777.5, premrna_ENST00000438223.5, premrna_ENST00000440425.5, premrna_ENST00000455608.5, premrna_ENST00000460658.5, premrna_ENST00000466272.1, premrna_ENST00000472911.1, premrna_ENST00000475548.5, premrna_ENST00000482444.5, premrna_ENST00000489337.5, premrna_ENST00000493335.5, premrna_ENST00000497697.5, premrna_ENST00000504335.1, premrna_ENST00000612341.4, premrna_ENST00000619644.4, and premrna_ENST00000624247.1. In some embodiments, the ASO targets a SMTN pre-mRNA sequence comprising a NSAE exon. In some embodiments, the ASO targets a SMTN pre-mRNA sequence comprising an intron flanking the 3′ splice site of the NSAE exon. In some embodiments, the ASO targets a SMTN pre-mRNA sequence comprising an intron flanking the 5′ splice site of the NSAE exon. In some embodiments, the transcript is selected from the group consisting of transcript_ENST00000333137.11, transcript_ENST00000347557.6, transcript_ENST00000358743.5, transcript_ENST00000404574.5, transcript_ENST00000416786.5, transcript_ENST00000422839.5, transcript_ENST00000426927.5, transcript_ENST00000431481.1, transcript_ENST00000432777.5, transcript_ENST00000438223.5, transcript_ENST00000440425.5, transcript_ENST00000455608.5, transcript_ENST00000460658.5, transcript_ENST00000466272.1, transcript_ENST00000472911.1, transcript_ENST00000475548.5, transcript_ENST00000482444.5, transcript_ENST00000489337.5, transcript_ENST00000493335.5, transcript_ENST00000497697.5, transcript_ENST00000504335.1, transcript_ENST00000612341.4, transcript_ENST00000619644.4, and transcript_ENST00000624247.1.

In some embodiments, the SMTN pre-mRNA transcript is encoded by a genetic sequence with at least about 80%, 85%, 90%, 95%, 96%, 97%, 98%, 99% or 100% sequence identity to the Ensembl reference number ENSG00000183963.18 or a complement thereof. In some embodiments, the SMTN pre-mRNA transcript comprises a sequence with at least about 80%, 85%, 90%, 95%, 96%, 97%, 98%, 99% or 100% sequence identity to a SMTN pre-mRNA transcript or a complement thereof described herein.

In some embodiments, the targeted portion of the SMTN pre-mRNA comprises a sequence with at least 80%, 85%, 90%, 95%, 97%, or 100% sequence identity to a region comprising at least 8 contiguous nucleic acids of a sequence of premrna_ENST00000333137.11, premrna_ENST00000347557.6, premrna_ENST00000358743.5, premrna_ENST00000404574.5, premrna_ENST00000416786.5, premrna_ENST00000422839.5, premrna_ENST00000426927.5, premrna_ENST00000431481.1, premrna_ENST00000432777.5, premrna_ENST00000438223.5, premrna_ENST00000440425.5, premrna_ENST00000455608.5, premrna_ENST00000460658.5, premrna_ENST00000466272.1, premrna_ENST00000472911.1, premrna_ENST00000475548.5, premrna_ENST00000482444.5, premrna_ENST00000489337.5, premrna_ENST00000493335.5, premrna_ENST00000497697.5, premrna_ENST00000504335.1, premrna_ENST00000612341.4, premrna_ENST00000619644.4, premrna_ENST00000624247.1 or a sequence of Table 3 or complements thereof. In some embodiments, the targeted portion of the SMTN pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 952-SEQ ID NO: 959 or complements thereof. In some embodiments, the targeted portion of the SMTN pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SNRPB genomic sequence. In some embodiments, the ASO targets a NSAE pre-mRNA transcript from a SNRPB genomic sequence comprising a NSAE exon. In some embodiments, the ASO targets a NSAE pre-mRNA transcript from a SNRPB genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000381342.6, premrna_ENST00000438552.6, and premrna_ENST00000474384.2. In some embodiments, the ASO targets a SNRPB pre-mRNA sequence comprising a NSAE exon. In some embodiments, the ASO targets a SNRPB pre-mRNA sequence comprising an intron flanking the 3′ splice site of the NSAE exon. In some embodiments, the ASO targets a SNRPB pre-mRNA sequence comprising an intron flanking the 5′ splice site of the NSAE exon. In some embodiments, the transcript is selected from the group consisting of transcript_ENST00000381342.6, transcript_ENST00000438552.6, and transcript_ENST00000474384.2.

In some embodiments, the SNRPB pre-mRNA transcript is encoded by a genetic sequence with at least about 80%, 85%, 90%, 95%, 96%, 97%, 98%, 99% or 100% sequence identity to the Ensembl reference number ENSG00000125835.19 or a complement thereof. In some embodiments, the SNRPB pre-mRNA transcript comprises a sequence with at least about 80%, 85%, 90%, 95%, 96%, 97%, 98%, 99% or 100% sequence identity to a SNRPB pre-mRNA transcript or a complement thereof described herein.

In some embodiments, the targeted portion of the SNRPB pre-mRNA comprises a sequence with at least 80%, 85%, 90%, 95%, 97%, or 100% sequence identity to a region comprising at least 8 contiguous nucleic acids of a sequence of premrna_ENST00000381342.6, premrna_ENST00000438552.6, premrna_ENST00000474384.2 or a sequence of Table 3 or complements thereof. In some embodiments, the targeted portion of the SNRPB pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 960-SEQ ID NO: 963 or complements thereof. In some embodiments, the targeted portion of the SNRPB pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SP140 genomic sequence. In some embodiments, the ASO targets a NSAE pre-mRNA transcript from a SP140 genomic sequence comprising a NSAE exon. In some embodiments, the ASO targets a NSAE pre-mRNA transcript from a SP140 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000343805.10, premrna_ENST00000373645.3, premrna_ENST00000392045.8, premrna_ENST00000417495.7, premrna_ENST00000420434.7, premrna_ENST00000441657.1, premrna_ENST00000456542.5, premrna_ENST00000473711.1, premrna_ENST00000476126.1, premrna_ENST00000479539.1, premrna_ENST00000486750.1, premrna_ENST00000538494.1, premrna_ENST00000543928.5, premrna_ENST00000544128.5. In some embodiments, the ASO targets a SP140 pre-mRNA sequence comprising a NSAE exon. In some embodiments, the ASO targets a SP140 pre-mRNA sequence comprising an intron flanking the 3′ splice site of the NSAE exon. In some embodiments, the ASO targets a SP140 pre-mRNA sequence comprising an intron flanking the 5′ splice site of the NSAE exon. In some embodiments, the transcript is selected from the group consisting of transcript_ENST00000343805.10, transcript_ENST00000373645.3, transcript_ENST00000392045.8, transcript_ENST00000417495.7, transcript_ENST00000420434.7, transcript_ENST00000441657.1, transcript_ENST00000456542.5, transcript_ENST00000473711.1, transcript_ENST00000476126.1, transcript_ENST00000479539.1, transcript_ENST00000486750.1, transcript_ENST00000538494.1, transcript_ENST00000543928.5, and transcript_ENST00000544128.5.

In some embodiments, the SP140 pre-mRNA transcript is encoded by a genetic sequence with at least about 80%, 85%, 90%, 95%, 96%, 97%, 98%, 99% or 100% sequence identity to the Ensembl reference number ENSG00000079263.19 or a complement thereof. In some embodiments, the SP140 pre-mRNA transcript comprises a sequence with at least about 80%, 85%, 90%, 95%, 96%, 97%, 98%, 99% or 100% sequence identity to a SP140 pre-mRNA transcript or a complement thereof described herein.

In some embodiments, the targeted portion of the SP140 pre-mRNA comprises a sequence with at least 80%, 85%, 90%, 95%, 97%, or 100% sequence identity to a region comprising at least 8 contiguous nucleic acids of a sequence of premrna_ENST00000343805.10, premrna_ENST00000373645.3, premrna_ENST00000392045.8, premrna_ENST00000417495.7, premrna_ENST00000420434.7, premrna_ENST00000441657.1, premrna_ENST00000456542.5, premrna_ENST00000473711.1, premrna_ENST00000476126.1, premrna_ENST00000479539.1, premrna_ENST00000486750.1, premrna_ENST00000538494.1, premrna_ENST00000543928.5, premrna_ENST00000544128.5 or a sequence of Table 3 or complements thereof. In some embodiments, the targeted portion of the SP140 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 964-SEQ ID NO: 967 or complements thereof. In some embodiments, the targeted portion of the SP140 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a STAMBP genomic sequence. In some embodiments, the ASO targets a NSAE pre-mRNA transcript from a STAMBP genomic sequence comprising a NSAE exon. In some embodiments, the ASO targets a NSAE pre-mRNA transcript from a STAMBP genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000339566.7, premrna_ENST00000394070.7, premrna_ENST00000394073.5, premrna_ENST00000409707.5, premrna_ENST00000424659.5, premrna_ENST00000432295.6, premrna_ENST00000452725.5, premrna_ENST00000478946.1, premrna_ENST00000486458.1, premrna_ENST00000487811.1, and premrna_ENST00000536064.1. In some embodiments, the ASO targets a STAMBP pre-mRNA sequence comprising a NSAE exon. In some embodiments, the ASO targets a STAMBP pre-mRNA sequence comprising an intron flanking the 3′ splice site of the NSAE exon. In some embodiments, the ASO targets a STAMBP pre-mRNA sequence comprising an intron flanking the 5′ splice site of the NSAE exon. In some embodiments, the transcript is selected from the group consisting of transcript_ENST00000339566.7, transcript_ENST00000394070.7, transcript_ENST00000394073.5, transcript_ENST00000409707.5, transcript_ENST00000424659.5, transcript_ENST00000432295.6, transcript_ENST00000452725.5, transcript_ENST00000478946.1, transcript_ENST00000486458.1, transcript_ENST00000487811.1, and transcript_ENST00000536064.1.

In some embodiments, the STAMBP pre-mRNA transcript is encoded by a genetic sequence with at least about 80%, 85%, 90%, 95%, 96%, 97%, 98%, 99% or 100% sequence identity to the Ensembl reference number ENSG00000124356.16 or a complement thereof. In some embodiments, the STAMBP pre-mRNA transcript comprises a sequence with at least about 80%, 85%, 90%, 95%, 96%, 97%, 98%, 99% or 100% sequence identity to a STAMBP pre-mRNA transcript or a complement thereof described herein.

In some embodiments, the targeted portion of the STAMBP pre-mRNA comprises a sequence with at least 80%, 85%, 90%, 95%, 97%, or 100% sequence identity to a region comprising at least 8 contiguous nucleic acids of a sequence of premrna_ENST00000339566.7, premrna_ENST00000394070.7, premrna_ENST00000394073.5, premrna_ENST00000409707.5, premrna_ENST00000424659.5, premrna_ENST00000432295.6, premrna_ENST00000452725.5, premrna_ENST00000478946.1, premrna_ENST00000486458.1, premrna_ENST00000487811.1, premrna_ENST00000536064.1 or a sequence of Table 3 or complements thereof. In some embodiments, the targeted portion of the STAMBP pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 968-SEQ ID NO: 971 or complements thereof. In some embodiments, the targeted portion of the STAMBP pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a STXBP2 genomic sequence. In some embodiments, the ASO targets a NSAE pre-mRNA transcript from a STXBP2 genomic sequence comprising a NSAE exon. In some embodiments, the ASO targets a NSAE pre-mRNA transcript from a STXBP2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000221283.10, premrna_ENST00000414284.6, premrna_ENST00000441779.6, premrna_ENST00000593535.5, premrna_ENST00000593854.5, premrna_ENST00000594221.5, premrna_ENST00000595181.5, premrna_ENST00000595800.1, premrna_ENST00000595861.1, premrna_ENST00000595950.5, premrna_ENST00000597068.5, premrna_ENST00000597467.1, premrna_ENST00000599278.1, premrna_ENST00000599400.1, premrna_ENST00000599558.1, premrna_ENST00000599648.1, premrna_ENST00000599737.5, premrna_ENST00000599905.1, premrna_ENST00000600702.5, premrna_ENST00000601061.1, premrna_ENST00000602355.1, and premrna_ENST00000612033.1. In some embodiments, the ASO targets a STXBP2 pre-mRNA sequence comprising a NSAE exon. In some embodiments, the ASO targets a STXBP2 pre-mRNA sequence comprising an intron flanking the 3′ splice site of the NSAE exon. In some embodiments, the ASO targets a STXBP2 pre-mRNA sequence comprising an intron flanking the 5′ splice site of the NSAE exon. In some embodiments, the transcript is selected from the group consisting of transcript_ENST00000221283.10, transcript_ENST00000414284.6, transcript_ENST00000441779.6, transcript_ENST00000593535.5, transcript_ENST00000593854.5, transcript_ENST00000594221.5, transcript_ENST00000595181.5, transcript_ENST00000595800.1, transcript_ENST00000595861.1, transcript_ENST00000595950.5, transcript_ENST00000597068.5, transcript_ENST00000597467.1, transcript_ENST00000599278.1, transcript_ENST00000599400.1, transcript_ENST00000599558.1, transcript_ENST00000599648.1, transcript_ENST00000599737.5, transcript_ENST00000599905.1, transcript_ENST00000600702.5, transcript_ENST00000601061.1, transcript_ENST00000602355.1, and transcript_ENST00000612033.1.

In some embodiments, the STXBP2 pre-mRNA transcript is encoded by a genetic sequence with at least about 80%, 85%, 90%, 95%, 96%, 97%, 98%, 99% or 100% sequence identity to the Ensembl reference number ENSG00000076944.16 or a complement thereof. In some embodiments, the STXBP2 pre-mRNA transcript comprises a sequence with at least about 80%, 85%, 90%, 95%, 96%, 97%, 98%, 99% or 100% sequence identity to a STXBP2 pre-mRNA transcript or a complement thereof described herein.

In some embodiments, the targeted portion of the STXBP2 pre-mRNA comprises a sequence with at least 80%, 85%, 90%, 95%, 97%, or 100% sequence identity to a region comprising at least 8 contiguous nucleic acids of a sequence of premrna_ENST00000221283.10, premrna_ENST00000414284.6, premrna_ENST00000441779.6, premrna_ENST00000593535.5, premrna_ENST00000593854.5, premrna_ENST00000594221.5, premrna_ENST00000595181.5, premrna_ENST00000595800.1, premrna_ENST00000595861.1, premrna_ENST00000595950.5, premrna_ENST00000597068.5, premrna_ENST00000597467.1, premrna_ENST00000599278.1, premrna_ENST00000599400.1, premrna_ENST00000599558.1, premrna_ENST00000599648.1, premrna_ENST00000599737.5, premrna_ENST00000599905.1, premrna_ENST00000600702.5, premrna_ENST00000601061.1, premrna_ENST00000602355.1, premrna_ENST00000612033.1 or a sequence of Table 3 or complements thereof. In some embodiments, the targeted portion of the STXBP2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 972-SEQ ID NO: 975 or complements thereof. In some embodiments, the targeted portion of the STXBP2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SULF2 genomic sequence. In some embodiments, the ASO targets a NSAE pre-mRNA transcript from a SULF2 genomic sequence comprising a NSAE exon. In some embodiments, the ASO targets a NSAE pre-mRNA transcript from a SULF2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000359930.8, premrna_ENST00000433632.1, premrna_ENST00000437955.1, premrna_ENST00000463221.2, premrna_ENST00000465769.1, premrna_ENST00000467815.5, premrna_ENST00000474450.5, premrna_ENST00000478766.1, premrna_ENST00000479472.1, premrna_ENST00000479970.1, premrna_ENST00000484875.5, and premrna_ENST00000495544.5. In some embodiments, the ASO targets a SULF2 pre-mRNA sequence comprising a NSAE exon. In some embodiments, the ASO targets a SULF2 pre-mRNA sequence comprising an intron flanking the 3′ splice site of the NSAE exon. In some embodiments, the ASO targets a SULF2 pre-mRNA sequence comprising an intron flanking the 5′ splice site of the NSAE exon. In some embodiments, the transcript is selected from the group consisting of transcript_ENST00000359930.8, transcript_ENST00000433632.1, transcript_ENST00000437955.1, transcript_ENST00000463221.2, transcript_ENST00000465769.1, transcript_ENST00000467815.5, transcript_ENST00000474450.5, transcript_ENST00000478766.1, transcript_ENST00000479472.1, transcript_ENST00000479970.1, transcript_ENST00000484875.5, and transcript_ENST00000495544.5.

In some embodiments, the SULF2 pre-mRNA transcript is encoded by a genetic sequence with at least about 80%, 85%, 90%, 95%, 96%, 97%, 98%, 99% or 100% sequence identity to the Ensembl reference number ENSG00000196562.14 or a complement thereof. In some embodiments, the SULF2 pre-mRNA transcript comprises a sequence with at least about 80%, 85%, 90%, 95%, 96%, 97%, 98%, 99% or 100% sequence identity to a SULF2 pre-mRNA transcript or a complement thereof described herein.

In some embodiments, the targeted portion of the SULF2 pre-mRNA comprises a sequence with at least 80%, 85%, 90%, 95%, 97%, or 100% sequence identity to a region comprising at least 8 contiguous nucleic acids of a sequence of premrna_ENST00000359930.8, premrna_ENST00000433632.1, premrna_ENST00000437955.1, premrna_ENST00000463221.2, premrna_ENST00000465769.1, premrna_ENST00000467815.5, premrna_ENST00000474450.5, premrna_ENST00000478766.1, premrna_ENST00000479472.1, premrna_ENST00000479970.1, premrna_ENST00000484875.5, premrna_ENST00000495544.5 or a sequence of Table 3 or complements thereof. In some embodiments, the targeted portion of the SULF2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 976-SEQ ID NO: 979 or complements thereof. In some embodiments, the targeted portion of the SULF2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SYNGAP1 genomic sequence. In some embodiments, the ASO targets a NSAE pre-mRNA transcript from a SYNGAP1 genomic sequence comprising a NSAE exon. In some embodiments, the ASO targets a NSAE pre-mRNA transcript from a SYNGAP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000293748.9, premrna_ENST00000418600.7, premrna_ENST00000428982.4, premrna_ENST00000449372.7, premrna_ENST00000470232.1, premrna_ENST00000479510.2, premrna_ENST00000628646.2, premrna_ENST00000629380.3, premrna_ENST00000635885.1, premrna_ENST00000636075.1, premrna_ENST00000636116.1, premrna_ENST00000636146.1, premrna_ENST00000636193.1, premrna_ENST00000636436.1, premrna_ENST00000636443.1, premrna_ENST00000636640.1, premrna_ENST00000636731.1, premrna_ENST00000636905.1, premrna_ENST00000637052.1, premrna_ENST00000637194.1, premrna_ENST00000637490.1, premrna_ENST00000637587.1, premrna_ENST00000637671.1, premrna_ENST00000637721.1, premrna_ENST00000637911.1, premrna_ENST00000638127.1, premrna_ENST00000638142.2, premrna_ENST00000644458.1, premrna_ENST00000645250.1, and premrna_ENST00000646630.1. In some embodiments, the ASO targets a SYNGAP1 pre-mRNA sequence comprising a NSAE exon. In some embodiments, the ASO targets a SYNGAP1 pre-mRNA sequence comprising an intron flanking the 3′ splice site of the NSAE exon. In some embodiments, the ASO targets a SYNGAP1 pre-mRNA sequence comprising an intron flanking the 5′ splice site of the NSAE exon. In some embodiments, the transcript is selected from the group consisting of transcript_ENST00000293748.9, transcript_ENST00000418600.7, transcript_ENST00000428982.4, transcript_ENST00000449372.7, transcript_ENST00000470232.1, transcript_ENST00000479510.2, transcript_ENST00000628646.2, transcript_ENST00000629380.3, transcript_ENST00000635885.1, transcript_ENST00000636075.1, transcript_ENST00000636116.1, transcript_ENST00000636146.1, transcript_ENST00000636193.1, transcript_ENST00000636436.1, transcript_ENST00000636443.1, transcript_ENST00000636640.1, transcript_ENST00000636731.1, transcript_ENST00000636905.1, transcript_ENST00000637052.1, transcript_ENST00000637194.1, transcript_ENST00000637490.1, transcript_ENST00000637587.1, transcript_ENST00000637671.1, transcript_ENST00000637721.1, transcript_ENST00000637911.1, transcript_ENST00000638127.1, transcript_ENST00000638142.2, transcript_ENST00000644458.1, transcript_ENST00000645250.1, and transcript_ENST00000646630.1.

In some embodiments, the SYNGAP1 pre-mRNA transcript is encoded by a genetic sequence with at least about 80%, 85%, 90%, 95%, 96%, 97%, 98%, 99% or 100% sequence identity to the Ensembl reference number ENSG00000197283.17, ENSG00000227460.8, or ENSG00000274259.2 or a complement thereof. In some embodiments, the SYNGAP1 pre-mRNA transcript comprises a sequence with at least about 80%, 85%, 90%, 95%, 96%, 97%, 98%, 99% or 100% sequence identity to a SYNGAP1 pre-mRNA transcript or a complement thereof described herein.

In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence of premrna_ENST00000293748.9, premrna_ENST00000418600.7, premrna_ENST00000428982.4, premrna_ENST00000449372.7, premrna_ENST00000470232.1, premrna_ENST00000479510.2, premrna_ENST00000628646.2, premrna_ENST00000629380.3, premrna_ENST00000635885.1, premrna_ENST00000636075.1, premrna_ENST00000636116.1, premrna_ENST00000636146.1, premrna_ENST00000636193.1, premrna_ENST00000636436.1, premrna_ENST00000636443.1, premrna_ENST00000636640.1, premrna_ENST00000636731.1, premrna_ENST00000636905.1, premrna_ENST00000637052.1, premrna_ENST00000637194.1, premrna_ENST00000637490.1, premrna_ENST00000637587.1, premrna_ENST00000637671.1, premrna_ENST00000637721.1, premrna_ENST00000637911.1, premrna_ENST00000638127.1, premrna_ENST00000638142.2, premrna_ENST00000644458.1, premrna_ENST00000645250.1, premrna_ENST00000646630.1 or a sequence of Table 2 or complements thereof. In some embodiments, the targeted portion of the SYNGAP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 980-SEQ ID NO: 983 or complements thereof. In some embodiments, the targeted portion of the SYNGAP1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a SYNJ2 genomic sequence. In some embodiments, the ASO targets a NSAE pre-mRNA transcript from a SYNJ2 genomic sequence comprising a NSAE exon. In some embodiments, the ASO targets a NSAE pre-mRNA transcript from a SYNJ2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000355585.9, premrna_ENST00000367112.1, premrna_ENST00000367113.5, premrna_ENST00000367122.6, premrna_ENST00000449320.2, premrna_ENST00000485863.1, premrna_ENST00000638626.1, premrna_ENST00000640338.1, and premrna_ENST00000640569.1. In some embodiments, the ASO targets a SYNJ2 pre-mRNA sequence comprising a NSAE exon. In some embodiments, the ASO targets a SYNJ2 pre-mRNA sequence comprising an intron flanking the 3′ splice site of the NSAE exon. In some embodiments, the ASO targets a SYNJ2 pre-mRNA sequence comprising an intron flanking the 5′ splice site of the NSAE exon. In some embodiments, the transcript is selected from the group consisting of transcript_ENST00000355585.9, transcript_ENST00000367112.1, transcript_ENST00000367113.5, transcript_ENST00000367122.6, transcript_ENST00000449320.2, transcript_ENST00000485863.1, transcript_ENST00000638626.1, transcript_ENST00000640338.1, and transcript_ENST00000640569.1.

In some embodiments, the SYNJ2 pre-mRNA transcript is encoded by a genetic sequence with at least about 80%, 85%, 90%, 95%, 96%, 97%, 98%, 99% or 100% sequence identity to the Ensembl reference number ENSG00000078269.15 or ENSG00000233496.1 or a complement thereof. In some embodiments, the SYNJ2 pre-mRNA transcript comprises a sequence with at least about 80%, 85%, 90%, 95%, 96%, 97%, 98%, 99% or 100% sequence identity to a SYNJ2 pre-mRNA transcript or a complement thereof described herein.

In some embodiments, the targeted portion of the SYNJ2 pre-mRNA comprises a sequence with at least 80%, 85%, 90%, 95%, 97%, or 100% sequence identity to a region comprising at least 8 contiguous nucleic acids of a sequence of premrna_ENST00000355585.9, premrna_ENST00000367112.1, premrna_ENST00000367113.5, premrna_ENST00000367122.6, premrna_ENST00000449320.2, premrna_ENST00000485863.1, premrna_ENST00000638626.1, premrna_ENST00000640338.1, premrna_ENST00000640569.1 or a sequence of Table 3 or complements thereof. In some embodiments, the targeted portion of the SYNJ2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 984-SEQ ID NO: 987 or complements thereof. In some embodiments, the targeted portion of the SYNJ2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TAZ genomic sequence. In some embodiments, the ASO targets a NSAE pre-mRNA transcript from a TAZ genomic sequence comprising a NSAE exon. In some embodiments, the ASO targets a NSAE pre-mRNA transcript from a TAZ genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000369776.8, premrna_ENST00000426231.5, premrna_ENST00000439735.2, premrna_ENST00000470127.2, premrna_ENST00000475699.6, premrna_ENST00000476679.5, premrna_ENST00000476800.2, premrna_ENST00000479875.1, premrna_ENST00000483674.3, premrna_ENST00000483780.5, premrna_ENST00000494912.5, premrna_ENST00000498029.1, premrna_ENST00000601016.6, premrna_ENST00000612012.5, premrna_ENST00000612460.5, premrna_ENST00000613002.4, premrna_ENST00000613634.4, premrna_ENST00000614595.2, premrna_ENST00000615658.5, premrna_ENST00000615986.4, premrna_ENST00000616020.5, premrna_ENST00000617701.5, premrna_ENST00000620808.4, premrna_ENST00000621647.2, premrna_ENST00000651139.1, premrna_ENST00000652354.1, premrna_ENST00000652358.1, premrna_ENST00000652390.1, premrna_ENST00000652476.1, premrna_ENST00000652644.1, premrna_ENST00000652682.1, and premrna_ENST00000652685.1. In some embodiments, the ASO targets a TAZ pre-mRNA sequence comprising a NSAE exon. In some embodiments, the ASO targets a TAZ pre-mRNA sequence comprising an intron flanking the 3′ splice site of the NSAE exon. In some embodiments, the ASO targets a TAZ pre-mRNA sequence comprising an intron flanking the 5′ splice site of the NSAE exon. In some embodiments, the transcript is selected from the group consisting of transcript_ENST00000369776.8, transcript_ENST00000426231.5, transcript_ENST00000439735.2, transcript_ENST00000470127.2, transcript_ENST00000475699.6, transcript_ENST00000476679.5, transcript_ENST00000476800.2, transcript_ENST00000479875.1, transcript_ENST00000483674.3, transcript_ENST00000483780.5, transcript_ENST00000494912.5, transcript_ENST00000498029.1, transcript_ENST00000601016.6, transcript_ENST00000612012.5, transcript_ENST00000612460.5, transcript_ENST00000613002.4, transcript_ENST00000613634.4, transcript_ENST00000614595.2, transcript_ENST00000615658.5, transcript_ENST00000615986.4, transcript_ENST00000616020.5, transcript_ENST00000617701.5, transcript_ENST00000620808.4, transcript_ENST00000621647.2, transcript_ENST00000651139.1, transcript_ENST00000652354.1, transcript_ENST00000652358.1, transcript_ENST00000652390.1, transcript_ENST00000652476.1, transcript_ENST00000652644.1, transcript_ENST00000652682.1, and transcript_ENST00000652685.1.

In some embodiments, the TAZ pre-mRNA transcript is encoded by a genetic sequence with at least about 80%, 85%, 90%, 95%, 96%, 97%, 98%, 99% or 100% sequence identity to the Ensembl reference number ENSG00000102125.16 or a complement thereof. In some embodiments, the TAZ pre-mRNA transcript comprises a sequence with at least about 80%, 85%, 90%, 95%, 96%, 97%, 98%, 99% or 100% sequence identity to a TAZ pre-mRNA transcript or a complement thereof described herein.

In some embodiments, the targeted portion of the TAZ pre-mRNA comprises a sequence with at least 80%, 85%, 90%, 95%, 97%, or 100% sequence identity to a region comprising at least 8 contiguous nucleic acids of a sequence of premrna_ENST00000369776.8, premrna_ENST00000426231.5, premrna_ENST00000439735.2, premrna_ENST00000470127.2, premrna_ENST00000475699.6, premrna_ENST00000476679.5, premrna_ENST00000476800.2, premrna_ENST00000479875.1, premrna_ENST00000483674.3, premrna_ENST00000483780.5, premrna_ENST00000494912.5, premrna_ENST00000498029.1, premrna_ENST00000601016.6, premrna_ENST00000612012.5, premrna_ENST00000612460.5, premrna_ENST00000613002.4, premrna_ENST00000613634.4, premrna_ENST00000614595.2, premrna_ENST00000615658.5, premrna_ENST00000615986.4, premrna_ENST00000616020.5, premrna_ENST00000617701.5, premrna_ENST00000620808.4, premrna_ENST00000621647.2, premrna_ENST00000651139.1, premrna_ENST00000652354.1, premrna_ENST00000652358.1, premrna_ENST00000652390.1, premrna_ENST00000652476.1, premrna_ENST00000652644.1, premrna_ENST00000652682.1, premrna_ENST00000652685.1, or a sequence of Table 3 or complements thereof. In some embodiments, the targeted portion of the TAZ pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 988-SEQ ID NO: 991 or complements thereof. In some embodiments, the targeted portion of the TAZ pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TBL2 genomic sequence. In some embodiments, the ASO targets a NSAE pre-mRNA transcript from a TBL2 genomic sequence comprising a NSAE exon. In some embodiments, the ASO targets a NSAE pre-mRNA transcript from a TBL2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000305632.11, premrna_ENST00000417008.5, premrna_ENST00000424598.5, premrna_ENST00000426966.5, premrna_ENST00000432538.5, premrna_ENST00000433464.5, premrna_ENST00000435792.5, premrna_ENST00000437521.5, premrna_ENST00000450285.5, premrna_ENST00000452125.5, premrna_ENST00000452475.5, premrna_ENST00000458466.1, premrna_ENST00000459913.5, premrna_ENST00000465279.5, premrna_ENST00000468669.5, premrna_ENST00000469518.1, premrna_ENST00000476136.5, premrna_ENST00000479892.5, premrna_ENST00000488915.1, premrna_ENST00000495885.1, premrna_ENST00000496056.5, and premrna_ENST00000610724.4. In some embodiments, the ASO targets a TBL2 pre-mRNA sequence comprising a NSAE exon. In some embodiments, the ASO targets a TBL2 pre-mRNA sequence comprising an intron flanking the 3′ splice site of the NSAE exon. In some embodiments, the ASO targets a TBL2 pre-mRNA sequence comprising an intron flanking the 5′ splice site of the NSAE exon. In some embodiments, the transcript is selected from the group consisting of transcript_ENST00000305632.11, transcript_ENST00000417008.5, transcript_ENST00000424598.5, transcript_ENST00000426966.5, transcript_ENST00000432538.5, transcript_ENST00000433464.5, transcript_ENST00000435792.5, transcript_ENST00000437521.5, transcript_ENST00000450285.5, transcript_ENST00000452125.5, transcript_ENST00000452475.5, transcript_ENST00000458466.1, transcript_ENST00000459913.5, transcript_ENST00000465279.5, transcript_ENST00000468669.5, transcript_ENST00000469518.1, transcript_ENST00000476136.5, transcript_ENST00000479892.5, transcript_ENST00000488915.1, transcript_ENST00000495885.1, transcript_ENST00000496056.5, and transcript_ENST00000610724.4.

In some embodiments, the TBL2 pre-mRNA transcript is encoded by a genetic sequence with at least about 80%, 85%, 90%, 95%, 96%, 97%, 98%, 99% or 100% sequence identity to the Ensembl reference number ENSG00000106638.17 or a complement thereof. In some embodiments, the TBL2 pre-mRNA transcript comprises a sequence with at least about 80%, 85%, 90%, 95%, 96%, 97%, 98%, 99% or 100% sequence identity to a TBL2 pre-mRNA transcript or a complement thereof described herein.

In some embodiments, the targeted portion of the TBL2 pre-mRNA comprises a sequence with at least 80%, 85%, 90%, 95%, 97%, or 100% sequence identity to a region comprising at least 8 contiguous nucleic acids of a sequence of premrna_ENST00000305632.11, premrna_ENST00000417008.5, premrna_ENST00000424598.5, premrna_ENST00000426966.5, premrna_ENST00000432538.5, premrna_ENST00000433464.5, premrna_ENST00000435792.5, premrna_ENST00000437521.5, premrna_ENST00000450285.5, premrna_ENST00000452125.5, premrna_ENST00000452475.5, premrna_ENST00000458466.1, premrna_ENST00000459913.5, premrna_ENST00000465279.5, premrna_ENST00000468669.5, premrna_ENST00000469518.1, premrna_ENST00000476136.5, premrna_ENST00000479892.5, premrna_ENST00000488915.1, premrna_ENST00000495885.1, premrna_ENST00000496056.5, premrna_ENST00000610724.4 or a sequence of Table 3 or complements thereof. In some embodiments, the targeted portion of the TBL2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 992-SEQ ID NO: 995 or complements thereof. In some embodiments, the targeted portion of the TBL2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TDRD7 genomic sequence. In some embodiments, the ASO targets a NSAE pre-mRNA transcript from a TDRD7 genomic sequence comprising a NSAE exon. In some embodiments, the ASO targets a NSAE pre-mRNA transcript from a TDRD7 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000355295.5, and premrna_ENST00000492428.1. In some embodiments, the ASO targets a TDRD7 pre-mRNA sequence comprising a NSAE exon. In some embodiments, the ASO targets a TDRD7 pre-mRNA sequence comprising an intron flanking the 3′ splice site of the NSAE exon. In some embodiments, the ASO targets a TDRD7 pre-mRNA sequence comprising an intron flanking the 5′ splice site of the NSAE exon. In some embodiments, the transcript is selected from the group consisting of transcript_ENST00000355295.5, and transcript_ENST00000492428.1.

In some embodiments, the TDRD7 pre-mRNA transcript is encoded by a genetic sequence with at least about 80%, 85%, 90%, 95%, 96%, 97%, 98%, 99% or 100% sequence identity to the Ensembl reference number ENSG00000196116.8 or a complement thereof. In some embodiments, the TDRD7 pre-mRNA transcript comprises a sequence with at least about 80%, 85%, 90%, 95%, 96%, 97%, 98%, 99% or 100% sequence identity to a TDRD7 pre-mRNA transcript or a complement thereof described herein.

In some embodiments, the targeted portion of the TDRD7 pre-mRNA comprises a sequence with at least 80%, 85%, 90%, 95%, 97%, or 100% sequence identity to a region comprising at least 8 contiguous nucleic acids of a sequence of premrna_ENST00000355295.5, premrna_ENST00000492428.1 or a sequence of Table 3 or complements thereof. In some embodiments, the targeted portion of the TDRD7 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 996-SEQ ID NO: 999 or complements thereof. In some embodiments, the targeted portion of the TDRD7 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TECPR2 genomic sequence. In some embodiments, the ASO targets a NSAE pre-mRNA transcript from a TECPR2 genomic sequence comprising a NSAE exon. In some embodiments, the ASO targets a NSAE pre-mRNA transcript from a TECPR2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000359520.12, premrna_ENST00000557786.1, premrna_ENST00000558678.1, premrna_ENST00000559124.1, premrna_ENST00000560060.5, premma_ENST00000561099.1, and premrna_ENST00000561228.1. In some embodiments, the ASO targets a TECPR2 pre-mRNA sequence comprising a NSAE exon. In some embodiments, the ASO targets a TECPR2 pre-mRNA sequence comprising an intron flanking the 3′ splice site of the NSAE exon. In some embodiments, the ASO targets a TECPR2 pre-mRNA sequence comprising an intron flanking the 5′ splice site of the NSAE exon. In some embodiments, the transcript is selected from the group consisting of transcript_ENST00000359520.12, transcript_ENST00000557786.1, transcript_ENST00000558678.1, transcript_ENST00000559124.1, transcript_ENST00000560060.5, transcript_ENST00000561099.1, and transcript_ENST00000561228.1.

In some embodiments, the TECPR2 pre-mRNA transcript is encoded by a genetic sequence with at least about 80%, 85%, 90%, 95%, 96%, 97%, 98%, 99% or 100% sequence identity to the Ensembl reference number ENSG00000196663.16 or a complement thereof. In some embodiments, the TECPR2 pre-mRNA transcript comprises a sequence with at least about 80%, 85%, 90%, 95%, 96%, 97%, 98%, 99% or 100% sequence identity to a TECPR2 pre-mRNA transcript or a complement thereof described herein.

In some embodiments, the targeted portion of the TECPR2 pre-mRNA comprises a sequence with at least 80%, 85%, 90%, 95%, 97%, or 100% sequence identity to a region comprising at least 8 contiguous nucleic acids of a sequence of premrna_ENST00000359520.12, premrna_ENST00000557786.1, premrna_ENST00000558678.1, premrna_ENST00000559124.1, premrna_ENST00000560060.5, premrna_ENST00000561099.1, premrna_ENST00000561228.1 or a sequence of Table 3 or complements thereof. In some embodiments, the targeted portion of the TECPR2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1000-SEQ ID NO: 1003 or complements thereof. In some embodiments, the targeted portion of the TECPR2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TFB1M genomic sequence. In some embodiments, the ASO targets a NSAE pre-mRNA transcript from a TFB1M genomic sequence comprising a NSAE exon. In some embodiments, the ASO targets a NSAE pre-mRNA transcript from a TFB1M genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000367166.5, premrna_ENST00000466349.1, premrna_ENST00000468889.5, premrna_ENST00000470239.1, premrna_ENST00000475849.1, premrna_ENST00000480390.1, premrna_ENST00000487586.5, premrna_ENST00000489874.5, and premrna_ENST00000495806.1. In some embodiments, the ASO targets a TFB1M pre-mRNA sequence comprising a NSAE exon. In some embodiments, the ASO targets a TFB1M pre-mRNA sequence comprising an intron flanking the 3′ splice site of the NSAE exon. In some embodiments, the ASO targets a TFB1M pre-mRNA sequence comprising an intron flanking the 5′ splice site of the NSAE exon. In some embodiments, the transcript is selected from the group consisting of transcript_ENST00000367166.5, transcript_ENST00000466349.1, transcript_ENST00000468889.5, transcript_ENST00000470239.1, transcript_ENST00000475849.1, transcript_ENST00000480390.1, transcript_ENST00000487586.5, transcript_ENST00000489874.5, and transcript_ENST00000495806.1.

In some embodiments, the TFB1M pre-mRNA transcript is encoded by a genetic sequence with at least about 80%, 85%, 90%, 95%, 96%, 97%, 98%, 99% or 100% sequence identity to the Ensembl reference number ENSG00000029639.11 or a complement thereof. In some embodiments, the TFB1M pre-mRNA transcript comprises a sequence with at least about 80%, 85%, 90%, 95%, 96%, 97%, 98%, 99% or 100% sequence identity to a TFB1M pre-mRNA transcript or a complement thereof described herein.

In some embodiments, the targeted portion of the TFB1M pre-mRNA comprises a sequence with at least 80%, 85%, 90%, 95%, 97%, or 100% sequence identity to a region comprising at least 8 contiguous nucleic acids of a sequence of premrna_ENST00000367166.5, premrna_ENST00000466349.1, premrna_ENST00000468889.5, premrna_ENST00000470239.1, premrna_ENST00000475849.1, premrna_ENST00000480390.1, premrna_ENST00000487586.5, premrna_ENST00000489874.5, premrna_ENST00000495806.1 or a sequence of Table 3 or complements thereof. In some embodiments, the targeted portion of the TFB1M pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1004-SEQ ID NO: 1007 or complements thereof. In some embodiments, the targeted portion of the TFB1M pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TM6SF1 genomic sequence. In some embodiments, the ASO targets a NSAE pre-mRNA transcript from a TM6SF1 genomic sequence comprising a NSAE exon. In some embodiments, the ASO targets a NSAE pre-mRNA transcript from a TM6SF1 genomic sequence comprising an intron flanking the 3′ splice site of the NSAE pre-mRNA transcribed from a TM6SF1 genomic sequence exon and an intron flanking the 5′ splice site of a NSAE exon. In some embodiments, the ASO targets a NSAE pre-mRNA transcript comprising a sequence selected from the group consisting of premrna_ENST00000258909.13, premrna_ENST00000322019.14, premrna_ENST00000379384.9, premrna_ENST00000379390.10, premrna_ENST00000561551.1, premrna_ENST00000564988.5, premrna_ENST00000565774.5, and premrna_ENST00000565982.1. In some embodiments, the ASO targets a TM6SF1 pre-mRNA sequence comprising a NSAE exon. In some embodiments, the ASO targets a TM6SF1 pre-mRNA sequence comprising an intron flanking the 3′ splice site of the NSAE exon. In some embodiments, the ASO targets a TM6SF1 pre-mRNA sequence comprising an intron flanking the 5′ splice site of the NSAE exon. In some embodiments, the transcript is selected from the group consisting of transcript_ENST00000258909.13, transcript_ENST00000322019.14, transcript_ENST00000379384.9, transcript_ENST00000379390.10, transcript_ENST00000561551.1, transcript_ENST00000564988.5, transcript_ENST00000565774.5, and transcript_ENST00000565982.1.

In some embodiments, the TM6SF1 pre-mRNA transcript is encoded by a genetic sequence with at least about 80%, 85%, 90%, 95%, 96%, 97%, 98%, 99% or 100% sequence identity to the Ensembl reference number ENSG00000136404.16 or a complement thereof. In some embodiments, the TM6SF1 pre-mRNA transcript comprises a sequence with at least about 80%, 85%, 90%, 95%, 96%, 97%, 98%, 99% or 100% sequence identity to a TM6SF1 pre-mRNA transcript or a complement thereof described herein.

In some embodiments, the targeted portion of the TM6SF1 pre-mRNA comprises a sequence with at least 80%, 85%, 90%, 95%, 97%, or 100% sequence identity to a region comprising at least 8 contiguous nucleic acids of a sequence of premrna_ENST00000258909.13, premrna_ENST00000322019.14, premrna_ENST00000379384.9, premrna_ENST00000379390.10, premrna_ENST00000561551.1, premrna_ENST00000564988.5, premrna_ENST00000565774.5, premrna_ENST00000565982.1 or a sequence of Table 3 or complements thereof. In some embodiments, the targeted portion of the TM6SF1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1008-SEQ ID NO: 1011 or complements thereof. In some embodiments, the targeted portion of the TM6SF1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TNK2 genomic sequence. In some embodiments, the ASO targets a NSAE pre-mRNA transcript from a TNK2 genomic sequence comprising a NSAE exon. In some embodiments, the ASO targets a NSAE pre-mRNA transcript from a TNK2 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000333602.13, premrna_ENST00000381916.7, premrna_ENST00000428187.7, premrna_ENST00000439230.6, premrna_ENST00000671726.1, premrna_ENST00000671734.1, premrna_ENST00000671753.1, premrna_ENST00000671767.1, premrna_ENST00000671831.1, premrna_ENST00000671880.1, premrna_ENST00000672024.1, premrna_ENST00000672098.1, premrna_ENST00000672145.1, premrna_ENST00000672320.1, premrna_ENST00000672542.1, premrna_ENST00000672548.1, premrna_ENST00000672614.1, premrna_ENST00000672623.1, premrna_ENST00000672669.1, premrna_ENST00000672886.1, premrna_ENST00000672887.1, premrna_ENST00000673038.1, premrna_ENST00000673167.1, premrna_ENST00000673236.1, premrna_ENST00000673358.1, premrna_ENST00000673374.1, premrna_ENST00000673420.1, premrna_ENST00000673440.1, premrna_ENST00000673443.1, and premrna_ENST00000673559.1. In some embodiments, the ASO targets a TNK2 pre-mRNA sequence comprising a NSAE exon. In some embodiments, the ASO targets a TNK2 pre-mRNA sequence comprising an intron flanking the 3′ splice site of the NSAE exon. In some embodiments, the ASO targets a TNK2 pre-mRNA sequence comprising an intron flanking the 5′ splice site of the NSAE exon. In some embodiments, the transcript is selected from the group consisting of transcript_ENST00000333602.13, transcript_ENST00000381916.7, transcript_ENST00000428187.7, transcript_ENST00000439230.6, transcript_ENST00000671726.1, transcript_ENST00000671734.1, transcript_ENST00000671753.1, transcript_ENST00000671767.1, transcript_ENST00000671831.1, transcript_ENST00000671880.1, transcript_ENST00000672024.1, transcript_ENST00000672098.1, transcript_ENST00000672145.1, transcript_ENST00000672320.1, transcript_ENST00000672542.1, transcript_ENST00000672548.1, transcript_ENST00000672614.1, transcript_ENST00000672623.1, transcript_ENST00000672669.1, transcript_ENST00000672886.1, transcript_ENST00000672887.1, transcript_ENST00000673038.1, transcript_ENST00000673167.1, transcript_ENST00000673236.1, transcript_ENST00000673358.1, transcript_ENST00000673374.1, transcript_ENST00000673420.1, transcript_ENST00000673440.1, transcript_ENST00000673443.1, and transcript_ENST00000673559.1.

In some embodiments, the TNK2 pre-mRNA transcript is encoded by a genetic sequence with at least about 80%, 85%, 90%, 95%, 96%, 97%, 98%, 99% or 100% sequence identity to the Ensembl reference number ENSG00000061938.19 or ENSG00000224614.2 or a complement thereof. In some embodiments, the TNK2 pre-mRNA transcript comprises a sequence with at least about 80%, 85%, 90%, 95%, 96%, 97%, 98%, 99% or 100% sequence identity to a TNK2 pre-mRNA transcript or a complement thereof described herein.

In some embodiments, the targeted portion of the TNK2 pre-mRNA comprises a sequence with at least 80%, 85%, 90%, 95%, 97%, or 100% sequence identity to a region comprising at least 8 contiguous nucleic acids of a sequence of premrna_ENST00000333602.13, premrna_ENST00000381916.7, premrna_ENST00000428187.7, premrna_ENST00000439230.6, premrna_ENST00000671726.1, premrna_ENST00000671734.1, premrna_ENST00000671753.1, premrna_ENST00000671767.1, premrna_ENST00000671831.1, premrna_ENST00000671880.1, premrna_ENST00000672024.1, premrna_ENST00000672098.1, premrna_ENST00000672145.1, premrna_ENST00000672320.1, premrna_ENST00000672542.1, premrna_ENST00000672548.1, premrna_ENST00000672614.1, premrna_ENST00000672623.1, premrna_ENST00000672669.1, premrna_ENST00000672886.1, premrna_ENST00000672887.1, premrna_ENST00000673038.1, premrna_ENST00000673167.1, premrna_ENST00000673236.1, premrna_ENST00000673358.1, premrna_ENST00000673374.1, premrna_ENST00000673420.1, premrna_ENST00000673440.1, premrna_ENST00000673443.1, premrna_ENST00000673559.1 or a sequence of Table 3 or complements thereof. In some embodiments, the targeted portion of the TNK2 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1012-SEQ ID NO: 1015 or complements thereof. In some embodiments, the targeted portion of the TNK2 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TOE1 genomic sequence. In some embodiments, the ASO targets a NSAE pre-mRNA transcript from a TOE1 genomic sequence comprising a NSAE exon. In some embodiments, the ASO targets a NSAE pre-mRNA transcript from a TOE1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000372090.6, premrna_ENST00000460057.1, premrna_ENST00000471337.5, premrna_ENST00000477731.5, and premrna_ENST00000495703.5. In some embodiments, the ASO targets a TOE1 pre-mRNA sequence comprising a NSAE exon. In some embodiments, the ASO targets a TOE1 pre-mRNA sequence comprising an intron flanking the 3′ splice site of the NSAE exon. In some embodiments, the ASO targets a TOE1 pre-mRNA sequence comprising an intron flanking the 5′ splice site of the NSAE exon. In some embodiments, the transcript is selected from the group consisting of transcript_ENST00000372090.6, transcript_ENST00000460057.1, transcript_ENST00000471337.5, transcript_ENST00000477731.5, and transcript_ENST00000495703.5.

In some embodiments, the TOE1 pre-mRNA transcript is encoded by a genetic sequence with at least about 80%, 85%, 90%, 95%, 96%, 97%, 98%, 99% or 100% sequence identity to the Ensembl reference number ENSG00000132773.12 or a complement thereof. In some embodiments, the TOE1 pre-mRNA transcript comprises a sequence with at least about 80%, 85%, 90%, 95%, 96%, 97%, 98%, 99% or 100% sequence identity to a TOE1 pre-mRNA transcript or a complement thereof described herein.

In some embodiments, the targeted portion of the TOE1 pre-mRNA comprises a sequence with at least 80%, 85%, 90%, 95%, 97%, or 100% sequence identity to a region comprising at least 8 contiguous nucleic acids of a sequence of premrna_ENST00000372090.6, premrna_ENST00000460057.1, premrna_ENST00000471337.5, premrna_ENST00000477731.5, premrna_ENST00000495703.5 or a sequence of Table 3 or complements thereof. In some embodiments, the targeted portion of the TOE1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1016-SEQ ID NO: 1019 or complements thereof. In some embodiments, the targeted portion of the TOE1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TOR1B genomic sequence. In some embodiments, the ASO targets a NSAE pre-mRNA transcript from a TOR1B genomic sequence comprising a NSAE exon. In some embodiments, the ASO targets a NSAE pre-mRNA transcript from a TOR1B genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000259339.7, premrna_ENST00000427860.1, premrna_ENST00000486372.1, and premrna_ENST00000488169.1. In some embodiments, the ASO targets a TOR1B pre-mRNA sequence comprising a NSAE exon. In some embodiments, the ASO targets a TOR1B pre-mRNA sequence comprising an intron flanking the 3′ splice site of the NSAE exon. In some embodiments, the ASO targets a TOR1B pre-mRNA sequence comprising an intron flanking the 5′ splice site of the NSAE exon. In some embodiments, the transcript is selected from the group consisting of transcript_ENST00000259339.7, transcript_ENST00000427860.1, transcript_ENST00000486372.1, and transcript_ENST00000488169.1.

In some embodiments, the TOR1B pre-mRNA transcript is encoded by a genetic sequence with at least about 80%, 85%, 90%, 95%, 96%, 97%, 98%, 99% or 100% sequence identity to the Ensembl reference number ENSG00000136816.16 or a complement thereof. In some embodiments, the TOR1B pre-mRNA transcript comprises a sequence with at least about 80%, 85%, 90%, 95%, 96%, 97%, 98%, 99% or 100% sequence identity to a TOR1B pre-mRNA transcript or a complement thereof described herein.

In some embodiments, the targeted portion of the TOR1B pre-mRNA comprises a sequence with at least 80%, 85%, 90%, 95%, 97%, or 100% sequence identity to a region comprising at least 8 contiguous nucleic acids of a sequence of premrna_ENST00000259339.7, premrna_ENST00000427860.1, premrna_ENST00000486372.1, premrna_ENST00000488169.1, or a sequence of Table 3 or complements thereof. In some embodiments, the targeted portion of the TOR1B pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1020-SEQ ID NO: 1023 or complements thereof. In some embodiments, the targeted portion of the TOR1B pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TRAPPC6B genomic sequence. In some embodiments, the ASO targets a NSAE pre-mRNA transcript from a TRAPPC6B genomic sequence comprising a NSAE exon. In some embodiments, the ASO targets a NSAE pre-mRNA transcript from a TRAPPC6B genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000330149.10, premrna_ENST00000347691.9, premrna_ENST00000469361.5, premrna_ENST00000554018.1, premrna_ENST00000555269.5, premrna_ENST00000556765.1, and premrna_ENST00000557764.5. In some embodiments, the ASO targets a TRAPPC6B pre-mRNA sequence comprising a NSAE exon. In some embodiments, the ASO targets a TRAPPC6B pre-mRNA sequence comprising an intron flanking the 3′ splice site of the NSAE exon. In some embodiments, the ASO targets a TRAPPC6B pre-mRNA sequence comprising an intron flanking the 5′ splice site of the NSAE exon. In some embodiments, the transcript is selected from the group consisting of transcript_ENST00000330149.10, transcript_ENST00000347691.9, transcript_ENST00000469361.5, transcript_ENST00000554018.1, transcript_ENST00000555269.5, transcript_ENST00000556765.1, and transcript_ENST00000557764.5.

In some embodiments, the TRAPPC6B pre-mRNA transcript is encoded by a genetic sequence with at least about 80%, 85%, 90%, 95%, 96%, 97%, 98%, 99% or 100% sequence identity to the Ensembl reference number

ENSG00000182400.15 or a complement thereof. In some embodiments, the TRAPPC6B pre-mRNA transcript comprises a sequence with at least about 80%, 85%, 90%, 95%, 96%, 97%, 98%, 99% or 100% sequence identity to a TRAPPC6B pre-mRNA transcript or a complement thereof described herein.

In some embodiments, the targeted portion of the TRAPPC6B pre-mRNA comprises a sequence with at least 80%, 85%, 90%, 95%, 97%, or 100% sequence identity to a region comprising at least 8 contiguous nucleic acids of a sequence of premrna_ENST00000330149.10, premrna_ENST00000347691.9, premrna_ENST00000469361.5, premrna_ENST00000554018.1, premrna_ENST00000555269.5, premrna_ENST00000556765.1, premrna_ENST00000557764.5 or a sequence of Table 3 or complements thereof. In some embodiments, the targeted portion of the TRAPPC6B pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1024-SEQ ID NO: 1027 or complements thereof. In some embodiments, the targeted portion of the TRAPPC6B pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TRIP4 genomic sequence. In some embodiments, the ASO targets a NSAE pre-mRNA transcript from a TRIP4 genomic sequence comprising a NSAE exon. In some embodiments, the ASO targets a NSAE pre-mRNA transcript from a TRIP4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000261884.8, premrna_ENST00000557834.5, premrna_ENST00000558162.1, premrna_ENST00000558442.1, premrna_ENST00000558820.5, premrna_ENST00000559565.5, premrna_ENST00000559777.1, premrna_ENST00000559833.5, premrna_ENST00000560475.1, premrna_ENST00000560567.5, premrna_ENST00000560920.6, and premrna_ENST00000561265.1. In some embodiments, the ASO targets a TRIP4 pre-mRNA sequence comprising a NSAE exon. In some embodiments, the ASO targets a TRIP4 pre-mRNA sequence comprising an intron flanking the 3′ splice site of the NSAE exon. In some embodiments, the ASO targets a TRIP4 pre-mRNA sequence comprising an intron flanking the 5′ splice site of the NSAE exon. In some embodiments, the transcript is selected from the group consisting of transcript_ENST00000261884.8, transcript_ENST00000557834.5, transcript_ENST00000558162.1, transcript_ENST00000558442.1, transcript_ENST00000558820.5, transcript_ENST00000559565.5, transcript_ENST00000559777.1, transcript_ENST00000559833.5, transcript_ENST00000560475.1, transcript_ENST00000560567.5, transcript_ENST00000560920.6, and transcript_ENST00000561265.1.

In some embodiments, the TRIP4 pre-mRNA transcript is encoded by a genetic sequence with at least about 80%, 85%, 90%, 95%, 96%, 97%, 98%, 99% or 100% sequence identity to the Ensembl reference number ENSG00000103671.10 or a complement thereof. In some embodiments, the TRIP4 pre-mRNA transcript comprises a sequence with at least about 80%, 85%, 90%, 95%, 96%, 97%, 98%, 99% or 100% sequence identity to a TRIP4 pre-mRNA transcript or a complement thereof described herein.

In some embodiments, the targeted portion of the TRIP4 pre-mRNA comprises a sequence with at least 80%, 85%, 90%, 95%, 97%, or 100% sequence identity to a region comprising at least 8 contiguous nucleic acids of a sequence of premrna_ENST00000261884.8, premrna_ENST00000557834.5, premrna_ENST00000558162.1, premrna_ENST00000558442.1, premrna_ENST00000558820.5, premrna_ENST00000559565.5, premrna_ENST00000559777.1, premrna_ENST00000559833.5, premrna_ENST00000560475.1, premrna_ENST00000560567.5, premrna_ENST00000560920.6, premrna_ENST00000561265.1 or a sequence of Table 3 or complements thereof. In some embodiments, the targeted portion of the TRIP4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1028-SEQ ID NO: 1031 or complements thereof. In some embodiments, the targeted portion of the TRIP4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TRMT2A genomic sequence. In some embodiments, the ASO targets a NSAE pre-mRNA transcript from a TRMT2A genomic sequence comprising a NSAE exon. In some embodiments, the ASO targets a NSAE pre-mRNA transcript from a TRMT2A genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000252136.12, premrna_ENST00000403707.7, premrna_ENST00000404751.7, premrna_ENST00000439169.2, premrna_ENST00000444256.1, premrna_ENST00000444845.5, premrna_ENST00000445045.1, premrna_ENST00000459644.1, premrna_ENST00000463710.1, premrna_ENST00000464535.1, premrna_ENST00000468917.1, premrna_ENST00000471040.5, premrna_ENST00000480339.1, premrna_ENST00000480460.1, premrna_ENST00000487378.1, premrna_ENST00000487668.5, premrna_ENST00000488335.1, premrna_ENST00000492988.5, premrna_ENST00000494641.1, and premrna_ENST00000494820.5.

In some embodiments, the ASO targets a TRMT2A pre-mRNA sequence comprising a NSAE exon. In some embodiments, the ASO targets a TRMT2A pre-mRNA sequence comprising an intron flanking the 3′ splice site of the NSAE exon. In some embodiments, the ASO targets a TRMT2A pre-mRNA sequence comprising an intron flanking the 5′ splice site of the NSAE exon. In some embodiments, the transcript is selected from the group consisting of transcript_ENST00000252136.12, transcript_ENST00000403707.7, transcript_ENST00000404751.7, transcript_ENST00000439169.2, transcript_ENST00000444256.1, transcript_ENST00000444845.5, transcript_ENST00000445045.1, transcript_ENST00000459644.1, transcript_ENST00000463710.1, transcript_ENST00000464535.1, transcript_ENST00000468917.1, transcript_ENST00000471040.5, transcript_ENST00000480339.1, transcript_ENST00000480460.1, transcript_ENST00000487378.1, transcript_ENST00000487668.5, transcript_ENST00000488335.1, transcript_ENST00000492988.5, transcript_ENST00000494641.1, and transcript_ENST00000494820.5.

In some embodiments, the TRMT2A pre-mRNA transcript is encoded by a genetic sequence with at least about 80%, 85%, 90%, 95%, 96%, 97%, 98%, 99% or 100% sequence identity to the Ensembl reference number ENSG00000099899.15 or a complement thereof. In some embodiments, the TRMT2A pre-mRNA transcript comprises a sequence with at least about 80%, 85%, 90%, 95%, 96%, 97%, 98%, 99% or 100% sequence identity to a TRMT2A pre-mRNA transcript or a complement thereof described herein.

In some embodiments, the targeted portion of the TRMT2A pre-mRNA comprises a sequence with at least 80%, 85%, 90%, 95%, 97%, or 100% sequence identity to a region comprising at least 8 contiguous nucleic acids of a sequence of premrna_ENST00000252136.12, premrna_ENST00000403707.7, premrna_ENST00000404751.7, premrna_ENST00000439169.2, premrna_ENST00000444256.1, premrna_ENST00000444845.5, premrna_ENST00000445045.1, premrna_ENST00000459644.1, premrna_ENST00000463710.1, premrna_ENST00000464535.1, premrna_ENST00000468917.1, premrna_ENST00000471040.5, premrna_ENST00000480339.1, premrna_ENST00000480460.1, premrna_ENST00000487378.1, premrna_ENST00000487668.5, premrna_ENST00000488335.1, premrna_ENST00000492988.5, premrna_ENST00000494641.1, premrna_ENST00000494820.5, or a sequence of Table 3 or complements thereof. In some embodiments, the targeted portion of the TRMT2A pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1032-SEQ ID NO: 1035 or complements thereof. In some embodiments, the targeted portion of the TRMT2A pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TRMU genomic sequence. In some embodiments, the ASO targets a NSAE pre-mRNA transcript from a TRMU genomic sequence comprising a NSAE exon. In some embodiments, the ASO targets a NSAE pre-mRNA transcript from a TRMU genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000381019.3, premrna_ENST00000381021.7, premrna_ENST00000441818.5, premrna_ENST00000453630.5, premrna_ENST00000456595.5, premrna_ENST00000457572.5, premrna_ENST00000463785.1, premrna_ENST00000465378.6, premrna_ENST00000470831.1, premrna_ENST00000476901.1, premrna_ENST00000479648.1, premrna_ENST00000485175.5, premrna_ENST00000485559.1, premrna_ENST00000486620.5, premrna_ENST00000491612.1, premrna_ENST00000493556.2, premrna_ENST00000496831.5, premrna_ENST00000642562.1, premrna_ENST00000642923.1, premrna_ENST00000643137.1, premrna_ENST00000644006.1, premrna_ENST00000645026.1, premrna_ENST00000645190.1, and premrna_ENST00000647301.1. In some embodiments, the ASO targets a TRMU pre-mRNA sequence comprising a NSAE exon. In some embodiments, the ASO targets a TRMU pre-mRNA sequence comprising an intron flanking the 3′ splice site of the NSAE exon. In some embodiments, the ASO targets a TRMU pre-mRNA sequence comprising an intron flanking the 5′ splice site of the NSAE exon. In some embodiments, the transcript is selected from the group consisting of transcript_ENST00000381019.3, transcript_ENST00000381021.7, transcript_ENST00000441818.5, transcript_ENST00000453630.5, transcript_ENST00000456595.5, transcript_ENST00000457572.5, transcript_ENST00000463785.1, transcript_ENST00000465378.6, transcript_ENST00000470831.1, transcript_ENST00000476901.1, transcript_ENST00000479648.1, transcript_ENST00000485175.5, transcript_ENST00000485559.1, transcript_ENST00000486620.5, transcript_ENST00000491612.1, transcript_ENST00000493556.2, transcript_ENST00000496831.5, transcript_ENST00000642562.1, transcript_ENST00000642923.1, transcript_ENST00000643137.1, transcript_ENST00000644006.1, transcript_ENST00000645026.1, transcript_ENST00000645190.1, and transcript_ENST00000647301.1.

In some embodiments, the TRMU pre-mRNA transcript is encoded by a genetic sequence with at least about 80%, 85%, 90%, 95%, 96%, 97%, 98%, 99% or 100% sequence identity to the Ensembl reference number ENSG00000100416.15 or a complement thereof. In some embodiments, the TRMU pre-mRNA transcript comprises a sequence with at least about 80%, 85%, 90%, 95%, 96%, 97%, 98%, 99% or 100% sequence identity to a TRMU pre-mRNA transcript or a complement thereof described herein.

In some embodiments, the targeted portion of the TRMU pre-mRNA comprises a sequence with at least 80%, 85%, 90%, 95%, 97%, or 100% sequence identity to a region comprising at least 8 contiguous nucleic acids of a sequence of premrna_ENST00000381019.3, premrna_ENST00000381021.7, premrna_ENST00000441818.5, premrna_ENST00000453630.5, premrna_ENST00000456595.5, premrna_ENST00000457572.5, premrna_ENST00000463785.1, premrna_ENST00000465378.6, premrna_ENST00000470831.1, premrna_ENST00000476901.1, premrna_ENST00000479648.1, premrna_ENST00000485175.5, premrna_ENST00000485559.1, premrna_ENST00000486620.5, premrna_ENST00000491612.1, premrna_ENST00000493556.2, premrna_ENST00000496831.5, premrna_ENST00000642562.1, premrna_ENST00000642923.1, premrna_ENST00000643137.1, premrna_ENST00000644006.1, premrna_ENST00000645026.1, premrna_ENST00000645190.1, premrna_ENST00000647301.1 or a sequence of Table 3 or complements thereof. In some embodiments, the targeted portion of the TRMU pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1036-SEQ ID NO: 1043 or complements thereof. In some embodiments, the targeted portion of the TRMU pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TRPV4 genomic sequence. In some embodiments, the ASO targets a NSAE pre-mRNA transcript from a TRPV4 genomic sequence comprising a NSAE exon. In some embodiments, the ASO targets a NSAE pre-mRNA transcript from a TRPV4 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000261740.7, premrna_ENST00000418703.6, premrna_ENST00000536570.1, premrna_ENST00000536838.1, premrna_ENST00000537083.5, premrna_ENST00000538125.5, premrna_ENST00000541794.5, and premrna_ENST00000544971.5. In some embodiments, the ASO targets a TRPV4 pre-mRNA sequence comprising a NSAE exon. In some embodiments, the ASO targets a TRPV4 pre-mRNA sequence comprising an intron flanking the 3′ splice site of the NSAE exon. In some embodiments, the ASO targets a TRPV4 pre-mRNA sequence comprising an intron flanking the 5′ splice site of the NSAE exon. In some embodiments, the transcript is selected from the group consisting of transcript_ENST00000261740.7, transcript_ENST00000418703.6, transcript_ENST00000536570.1, transcript_ENST00000536838.1, transcript_ENST00000537083.5, transcript_ENST00000538125.5, transcript_ENST00000541794.5, and transcript_ENST00000544971.5.

In some embodiments, the TRPV4 pre-mRNA transcript is encoded by a genetic sequence with at least about 80%, 85%, 90%, 95%, 96%, 97%, 98%, 99% or 100% sequence identity to the Ensembl reference number ENSG00000111199.11 or a complement thereof. In some embodiments, the TRPV4 pre-mRNA transcript comprises a sequence with at least about 80%, 85%, 90%, 95%, 96%, 97%, 98%, 99% or 100% sequence identity to a TRPV4 pre-mRNA transcript or a complement thereof described herein.

In some embodiments, the targeted portion of the TRPV4 pre-mRNA comprises a sequence with at least 80%, 85%, 90%, 95%, 97%, or 100% sequence identity to a region comprising at least 8 contiguous nucleic acids of a sequence of premrna_ENST00000261740.7, premrna_ENST00000418703.6, premrna_ENST00000536570.1, premrna_ENST00000536838.1, premrna_ENST00000537083.5, premrna_ENST00000538125.5, premrna_ENST00000541794.5, premrna_ENST00000544971.5 or a sequence of Table 3 or complements thereof. In some embodiments, the targeted portion of the TRPV4 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1044-SEQ ID NO: 1047 or complements thereof. In some embodiments, the targeted portion of the TRPV4 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TTC19 genomic sequence. In some embodiments, the ASO targets a NSAE pre-mRNA transcript from a TTC19 genomic sequence comprising a NSAE exon. In some embodiments, the ASO targets a NSAE pre-mRNA transcript from a TTC19 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000261647.10, premrna_ENST00000465567.1, premrna_ENST00000466729.5, premrna_ENST00000470399.1, premrna_ENST00000470649.1, premrna_ENST00000475723.5, premrna_ENST00000481107.1, premrna_ENST00000497842.6, premma_ENST00000578103.1, and premrna_ENST00000583704.1. In some embodiments, the ASO targets a TTC19 pre-mRNA sequence comprising a NSAE exon. In some embodiments, the ASO targets a TTC19 pre-mRNA sequence comprising an intron flanking the 3′ splice site of the NSAE exon. In some embodiments, the ASO targets a TTC19 pre-mRNA sequence comprising an intron flanking the 5′ splice site of the NSAE exon. In some embodiments, the transcript is selected from the group consisting of transcript_ENST00000261647.10, transcript_ENST00000465567.1, transcript_ENST00000466729.5, transcript_ENST00000470399.1, transcript_ENST00000470649.1, transcript_ENST00000475723.5, transcript_ENST00000481107.1, transcript_ENST00000497842.6, transcript_ENST00000578103.1, and transcript_ENST00000583704.1.

In some embodiments, the TTC19 pre-mRNA transcript is encoded by a genetic sequence with at least about 80%, 85%, 90%, 95%, 96%, 97%, 98%, 99% or 100% sequence identity to the Ensembl reference number ENSG00000011295.16 or a complement thereof. In some embodiments, the TTC19 pre-mRNA transcript comprises a sequence with at least about 80%, 85%, 90%, 95%, 96%, 97%, 98%, 99% or 100% sequence identity to a TTC19 pre-mRNA transcript or a complement thereof described herein.

In some embodiments, the targeted portion of the TTC19 pre-mRNA comprises a sequence with at least 80%, 85%, 90%, 95%, 97%, or 100% sequence identity to a region comprising at least 8 contiguous nucleic acids of a sequence of premrna_ENST00000261647.10, premrna_ENST00000465567.1, premrna_ENST00000466729.5, premrna_ENST00000470399.1, premrna_ENST00000470649.1, premrna_ENST00000475723.5, premrna_ENST00000481107.1, premrna_ENST00000497842.6, premrna_ENST00000578103.1, premrna_ENST00000583704.1 or a sequence of Table 3 or complements thereof. In some embodiments, the targeted portion of the TTC19 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1048-SEQ ID NO: 1051 or complements thereof. In some embodiments, the targeted portion of the TTC19 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a TYMP genomic sequence. In some embodiments, the ASO targets a NSAE pre-mRNA transcript from a TYMP genomic sequence comprising a NSAE exon. In some embodiments, the ASO targets a NSAE pre-mRNA transcript from a TYMP genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000252029.8, premrna_ENST00000395678.7, premrna_ENST00000395680.6, premrna_ENST00000395681.6, premrna_ENST00000425169.1, premrna_ENST00000476284.1, premrna_ENST00000487162.1, premrna_ENST00000487577.5, premrna_ENST00000650719.1, premrna_ENST00000651095.1, premrna_ENST00000651196.1, premrna_ENST00000651401.1, premrna_ENST00000651490.1, premrna_ENST00000651906.1, premrna_ENST00000652237.1, premrna_ENST00000652352.1, and premrna_ENST00000652401.1. In some embodiments, the ASO targets a TYMP pre-mRNA sequence comprising a NSAE exon. In some embodiments, the ASO targets a TYMP pre-mRNA sequence comprising an intron flanking the 3′ splice site of the NSAE exon. In some embodiments, the ASO targets a TYMP pre-mRNA sequence comprising an intron flanking the 5′ splice site of the NSAE exon. In some embodiments, the transcript is selected from the group consisting of transcript_ENST00000252029.8, transcript_ENST00000395678.7, transcript_ENST00000395680.6, transcript_ENST00000395681.6, transcript_ENST00000425169.1, transcript_ENST00000476284.1, transcript_ENST00000487162.1, transcript_ENST00000487577.5, transcript_ENST00000650719.1, transcript_ENST00000651095.1, transcript_ENST00000651196.1, transcript_ENST00000651401.1, transcript_ENST00000651490.1, transcript_ENST00000651906.1, transcript_ENST00000652237.1, transcript_ENST00000652352.1, and transcript_ENST00000652401.1.

In some embodiments, the TYMP pre-mRNA transcript is encoded by a genetic sequence with at least about 80%, 85%, 90%, 95%, 96%, 97%, 98%, 99% or 100% sequence identity to the Ensembl reference number ENSG00000025708.14 or a complement thereof. In some embodiments, the TYMP pre-mRNA transcript comprises a sequence with at least about 80%, 85%, 90%, 95%, 96%, 97%, 98%, 99% or 100% sequence identity to a TYMP pre-mRNA transcript or a complement thereof described herein.

In some embodiments, the targeted portion of the TYMP pre-mRNA comprises a sequence with at least 80%, 85%, 90%, 95%, 97%, or 100% sequence identity to a region comprising at least 8 contiguous nucleic acids of a sequence of premrna_ENST00000252029.8, premrna_ENST00000395678.7, premrna_ENST00000395680.6, premrna_ENST00000395681.6, premrna_ENST00000425169.1, premrna_ENST00000476284.1, premrna_ENST00000487162.1, premrna_ENST00000487577.5, premrna_ENST00000650719.1, premrna_ENST00000651095.1, premrna_ENST00000651196.1, premrna_ENST00000651401.1, premrna_ENST00000651490.1, premrna_ENST00000651906.1, premrna_ENST00000652237.1, premrna_ENST00000652352.1, premrna_ENST00000652401.1 or a sequence of Table 3 or complements thereof. In some embodiments, the targeted portion of the TYMP pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1052-SEQ ID NO: 1057 or complements thereof. In some embodiments, the targeted portion of the TYMP pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a UMPS genomic sequence. In some embodiments, the ASO targets a NSAE pre-mRNA transcript from a UMPS genomic sequence comprising a NSAE exon. In some embodiments, the ASO targets a NSAE pre-mRNA transcript from a UMPS genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000232607.7, premrna_ENST00000460034.5, premrna_ENST00000462091.5, premrna_ENST00000467167.5, premrna_ENST00000474588.5, premrna_ENST00000479719.5, premrna_ENST00000487622.5, premrna_ENST00000495751.1, premrna_ENST00000497791.5, premrna_ENST00000498715.1, and premrna_ENST00000628619.1. In some embodiments, the ASO targets a UMPS pre-mRNA sequence comprising a NSAE exon. In some embodiments, the ASO targets a UMPS pre-mRNA sequence comprising an intron flanking the 3′ splice site of the NSAE exon. In some embodiments, the ASO targets a UMPS pre-mRNA sequence comprising an intron flanking the 5′ splice site of the NSAE exon. In some embodiments, the transcript is selected from the group consisting of transcript_ENST00000232607.7, transcript_ENST00000460034.5, transcript_ENST00000462091.5, transcript_ENST00000467167.5, transcript_ENST00000474588.5, transcript_ENST00000479719.5, transcript_ENST00000487622.5, transcript_ENST00000495751.1, transcript_ENST00000497791.5, transcript_ENST00000498715.1, and transcript_ENST00000628619.1.

In some embodiments, the UMPS pre-mRNA transcript is encoded by a genetic sequence with at least about 80%, 85%, 90%, 95%, 96%, 97%, 98%, 99% or 100% sequence identity to the Ensembl reference number ENSG00000114491.14 or a complement thereof. In some embodiments, the UMPS pre-mRNA transcript comprises a sequence with at least about 80%, 85%, 90%, 95%, 96%, 97%, 98%, 99% or 100% sequence identity to a UMPS pre-mRNA transcript or a complement thereof described herein.

In some embodiments, the targeted portion of the UMPS pre-mRNA comprises a sequence with at least 80%, 85%, 90%, 95%, 97%, or 100% sequence identity to a region comprising at least 8 contiguous nucleic acids of a sequence of premrna_ENST00000232607.7, premrna_ENST00000460034.5, premrna_ENST00000462091.5, premrna_ENST00000467167.5, premrna_ENST00000474588.5, premrna_ENST00000479719.5, premrna_ENST00000487622.5, premrna_ENST00000495751.1, premrna_ENST00000497791.5, premrna_ENST00000498715.1, premrna_ENST00000628619.1 or a sequence of Table 3 or complements thereof. In some embodiments, the targeted portion of the UMPS pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1058-SEQ ID NO: 1061 or complements thereof. In some embodiments, the targeted portion of the UMPS pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a UROD genomic sequence. In some embodiments, the ASO targets a NSAE pre-mRNA transcript from a UROD genomic sequence comprising a NSAE exon. In some embodiments, the ASO targets a NSAE pre-mRNA transcript from a UROD genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000246337.9, premrna_ENST00000428106.1, premrna_ENST00000434478.6, premrna_ENST00000460334.5, premrna_ENST00000460906.5, premrna_ENST00000461035.5, premrna_ENST00000462688.5, premrna_ENST00000463092.5, premrna_ENST00000465678.1, premrna_ENST00000466193.1, premrna_ENST00000469548.5, premrna_ENST00000472254.1, premrna_ENST00000473012.1, premrna_ENST00000478467.5, premrna_ENST00000486699.5, premrna_ENST00000490385.5, premrna_ENST00000491300.5, premrna_ENST00000491773.6, premrna_ENST00000494399.5, premrna_ENST00000496439.1, premrna_ENST00000636293.1, premrna_ENST00000636836.1, premrna_ENST00000650713.1, premrna_ENST00000651476.1, premrna_ENST00000652165.1, premrna_ENST00000652287.1, and premrna_ENST00000652514.1. In some embodiments, the ASO targets a UROD pre-mRNA sequence comprising a NSAE exon. In some embodiments, the ASO targets a UROD pre-mRNA sequence comprising an intron flanking the 3′ splice site of the NSAE exon. In some embodiments, the ASO targets a UROD pre-mRNA sequence comprising an intron flanking the 5′ splice site of the NSAE exon. In some embodiments, the transcript is selected from the group consisting of transcript_ENST00000246337.9, transcript_ENST00000428106.1, transcript_ENST00000434478.6, transcript_ENST00000460334.5, transcript_ENST00000460906.5, transcript_ENST00000461035.5, transcript_ENST00000462688.5, transcript_ENST00000463092.5, transcript_ENST00000465678.1, transcript_ENST00000466193.1, transcript_ENST00000469548.5, transcript_ENST00000472254.1, transcript_ENST00000473012.1, transcript_ENST00000478467.5, transcript_ENST00000486699.5, transcript_ENST00000490385.5, transcript_ENST00000491300.5, transcript_ENST00000491773.6, transcript_ENST00000494399.5, transcript_ENST00000496439.1, transcript_ENST00000636293.1, transcript_ENST00000636836.1, transcript_ENST00000650713.1, transcript_ENST00000651476.1, transcript_ENST00000652165.1, transcript_ENST00000652287.1, and transcript_ENST00000652514.1.

In some embodiments, the UROD pre-mRNA transcript is encoded by a genetic sequence with at least about 80%, 85%, 90%, 95%, 96%, 97%, 98%, 99% or 100% sequence identity to the Ensembl reference number ENSG00000126088.14 or a complement thereof. In some embodiments, the UROD pre-mRNA transcript comprises a sequence with at least about 80%, 85%, 90%, 95%, 96%, 97%, 98%, 99% or 100% sequence identity to a UROD pre-mRNA transcript or a complement thereof described herein.

In some embodiments, the targeted portion of the UROD pre-mRNA comprises a sequence with at least 80%, 85%, 90%, 95%, 97%, or 100% sequence identity to a region comprising at least 8 contiguous nucleic acids of a sequence of premrna_ENST00000246337.9, premrna_ENST00000428106.1, premrna_ENST00000434478.6, premrna_ENST00000460334.5, premrna_ENST00000460906.5, premrna_ENST00000461035.5, premrna_ENST00000462688.5, premrna_ENST00000463092.5, premrna_ENST00000465678.1, premrna_ENST00000466193.1, premrna_ENST00000469548.5, premrna_ENST00000472254.1, premrna_ENST00000473012.1, premrna_ENST00000478467.5, premrna_ENST00000486699.5, premrna_ENST00000490385.5, premrna_ENST00000491300.5, premrna_ENST00000491773.6, premrna_ENST00000494399.5, premrna_ENST00000496439.1, premrna_ENST00000636293.1, premrna_ENST00000636836.1, premrna_ENST00000650713.1, premrna_ENST00000651476.1, premrna_ENST00000652165.1, premrna_ENST00000652287.1, premrna_ENST00000652514.1 or a sequence of Table 3 or complements thereof. In some embodiments, the targeted portion of the UROD pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1062-SEQ ID NO: 1065 or complements thereof. In some embodiments, the targeted portion of the UROD pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a WDR11 genomic sequence. In some embodiments, the ASO targets a NSAE pre-mRNA transcript from a WDR11 genomic sequence comprising a NSAE exon. In some embodiments, the ASO targets a NSAE pre-mRNA transcript from a WDR11 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000263461.11, premrna_ENST00000462529.2, premrna_ENST00000470052.5, premrna_ENST00000478567.5, premrna_ENST00000497136.6, premrna_ENST00000603658.1, premrna_ENST00000604220.5, premrna_ENST00000604509.5, premrna_ENST00000604585.5, premrna_ENST00000604714.1, premrna_ENST00000605069.5, premrna_ENST00000605178.5, premrna_ENST00000605202.5, premrna_ENST00000605320.1, premrna_ENST00000605376.5, premrna_ENST00000605543.5, and premrna_ENST00000605659.1. In some embodiments, the ASO targets a WDR11 pre-mRNA sequence comprising a NSAE exon. In some embodiments, the ASO targets a WDR11 pre-mRNA sequence comprising an intron flanking the 3′ splice site of the NSAE exon. In some embodiments, the ASO targets a WDR11 pre-mRNA sequence comprising an intron flanking the 5′ splice site of the NSAE exon. In some embodiments, the transcript is selected from the group consisting of transcript_ENST00000263461.11, transcript_ENST00000462529.2, transcript_ENST00000470052.5, transcript_ENST00000478567.5, transcript_ENST00000497136.6, transcript_ENST00000603658.1, transcript_ENST00000604220.5, transcript_ENST00000604509.5, transcript_ENST00000604585.5, transcript_ENST00000604714.1, transcript_ENST00000605069.5, transcript_ENST00000605178.5, transcript_ENST00000605202.5, transcript_ENST00000605320.1, transcript_ENST00000605376.5, transcript_ENST00000605543.5, and transcript_ENST00000605659.1.

In some embodiments, the WDR11 pre-mRNA transcript is encoded by a genetic sequence with at least about 80%, 85%, 90%, 95%, 96%, 97%, 98%, 99% or 100% sequence identity to the Ensembl reference number ENSG00000120008.16 or a complement thereof. In some embodiments, the WDR11 pre-mRNA transcript comprises a sequence with at least about 80%, 85%, 90%, 95%, 96%, 97%, 98%, 99% or 100% sequence identity to a WDR11 pre-mRNA transcript or a complement thereof described herein.

In some embodiments, the targeted portion of the WDR11 pre-mRNA comprises a sequence with at least 80%, 85%, 90%, 95%, 97%, or 100% sequence identity to a region comprising at least 8 contiguous nucleic acids of a sequence of premrna_ENST00000263461.11, premrna_ENST00000462529.2, premrna_ENST00000470052.5, premrna_ENST00000478567.5, premrna_ENST00000497136.6, premrna_ENST00000603658.1, premrna_ENST00000604220.5, premrna_ENST00000604509.5, premrna_ENST00000604585.5, premrna_ENST00000604714.1, premrna_ENST00000605069.5, premrna_ENST00000605178.5, premrna_ENST00000605202.5, premrna_ENST00000605320.1, premrna_ENST00000605376.5, premrna_ENST00000605543.5, premrna_ENST00000605659.1 or a sequence of Table 3 or complements thereof. In some embodiments, the targeted portion of the WDR11 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1066-SEQ ID NO: 1069 or complements thereof. In some embodiments, the targeted portion of the WDR11 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a WDR62 genomic sequence. In some embodiments, the ASO targets a NSAE pre-mRNA transcript from a WDR62 genomic sequence comprising a NSAE exon. In some embodiments, the ASO targets a NSAE pre-mRNA transcript from a WDR62 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000270301.11, premrna_ENST00000378860.8, premrna_ENST00000401500.7, premrna_ENST00000427823.2, premrna_ENST00000587391.5, premrna_ENST00000589953.1, premrna_ENST00000608676.1, and premrna_ENST00000644764.1.

In some embodiments, the ASO targets a WDR62 pre-mRNA sequence comprising a NSAE exon. In some embodiments, the ASO targets a WDR62 pre-mRNA sequence comprising an intron flanking the 3′ splice site of the NSAE exon. In some embodiments, the ASO targets a WDR62 pre-mRNA sequence comprising an intron flanking the 5′ splice site of the NSAE exon. In some embodiments, the transcript is selected from the group consisting of transcript_ENST00000270301.11, transcript_ENST00000378860.8, transcript_ENST00000401500.7, transcript_ENST00000427823.2, transcript_ENST00000587391.5, transcript_ENST00000589953.1, transcript_ENST00000608676.1, and transcript_ENST00000644764.1.

In some embodiments, the WDR62 pre-mRNA transcript is encoded by a genetic sequence with at least about 80%, 85%, 90%, 95%, 96%, 97%, 98%, 99% or 100% sequence identity to the Ensembl reference number ENSG00000075702.18 or a complement thereof. In some embodiments, the WDR62 pre-mRNA transcript comprises a sequence with at least about 80%, 85%, 90%, 95%, 96%, 97%, 98%, 99% or 100% sequence identity to a WDR62 pre-mRNA transcript or a complement thereof described herein.

In some embodiments, the targeted portion of the WDR62 pre-mRNA comprises a sequence with at least 80%, 85%, 90%, 95%, 97%, or 100% sequence identity to a region comprising at least 8 contiguous nucleic acids of a sequence of premrna_ENST00000270301.11, premrna_ENST00000378860.8, premrna_ENST00000401500.7, premrna_ENST00000427823.2, premrna_ENST00000587391.5, premrna_ENST00000589953.1, premrna_ENST00000608676.1, premrna_ENST00000644764.1, or a sequence of Table 3 or complements thereof. In some embodiments, the targeted portion of the WDR62 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1070-SEQ ID NO: 1073 or complements thereof. In some embodiments, the targeted portion of the WDR62 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a WIPI1 genomic sequence. In some embodiments, the ASO targets a NSAE pre-mRNA transcript from a WIPI1 genomic sequence comprising a NSAE exon. In some embodiments, the ASO targets a NSAE pre-mRNA transcript from a WIPI1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000262139.10, premrna_ENST00000546360.5, premrna_ENST00000585393.1, premrna_ENST00000586815.1, premrna_ENST00000587731.1, premrna_ENST00000589316.5, premrna_ENST00000589459.5, premrna_ENST00000590402.1, premrna_ENST00000591494.5, premrna_ENST00000591744.1, and premrna_ENST00000592645.5. In some embodiments, the ASO targets a WIPI1 pre-mRNA sequence comprising a NSAE exon. In some embodiments, the ASO targets a WIPI1 pre-mRNA sequence comprising an intron flanking the 3′ splice site of the NSAE exon. In some embodiments, the ASO targets a WIPI1 pre-mRNA sequence comprising an intron flanking the 5′ splice site of the NSAE exon. In some embodiments, the transcript is selected from the group consisting of transcript_ENST00000262139.10, transcript_ENST00000546360.5, transcript_ENST00000585393.1, transcript_ENST00000586815.1, transcript_ENST00000587731.1, transcript_ENST00000589316.5, transcript_ENST00000589459.5, transcript_ENST00000590402.1, transcript_ENST00000591494.5, transcript_ENST00000591744.1, and transcript_ENST00000592645.5.

In some embodiments, the WIPI1 pre-mRNA transcript is encoded by a genetic sequence with at least about 80%, 85%, 90%, 95%, 96%, 97%, 98%, 99% or 100% sequence identity to the Ensembl reference number ENSG00000070540.13 or a complement thereof. In some embodiments, the WIPI1 pre-mRNA transcript comprises a sequence with at least about 80%, 85%, 90%, 95%, 96%, 97%, 98%, 99% or 100% sequence identity to a WIPI1 pre-mRNA transcript or a complement thereof described herein.

In some embodiments, the targeted portion of the WIPI1 pre-mRNA comprises a sequence with at least 80%, 85%, 90%, 95%, 97%, or 100% sequence identity to a region comprising at least 8 contiguous nucleic acids of a sequence of premrna_ENST00000262139.10, premrna_ENST00000546360.5, premrna_ENST00000585393.1, premrna_ENST00000586815.1, premrna_ENST00000587731.1, premrna_ENST00000589316.5, premrna_ENST00000589459.5, premrna_ENST00000590402.1, premrna_ENST00000591494.5, premrna_ENST00000591744.1, premrna_ENST00000592645.5 or a sequence of Table 3 or complements thereof. In some embodiments, the targeted portion of the WIPI1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1074-SEQ ID NO: 1077 or complements thereof. In some embodiments, the targeted portion of the WIPI1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a WRAP53 genomic sequence. In some embodiments, the ASO targets a NSAE pre-mRNA transcript from a WRAP53 genomic sequence comprising a NSAE exon. In some embodiments, the ASO targets a NSAE pre-mRNA transcript from a WRAP53 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000316024.9, premrna_ENST00000396463.6, premrna_ENST00000431639.6, premrna_ENST00000457584.6, premrna_ENST00000463804.6, premrna_ENST00000467699.5, premrna_ENST00000471973.6, premrna_ENST00000498114.1, premrna_ENST00000498311.5, and premrna_ENST00000534050.5. In some embodiments, the ASO targets a WRAP53 pre-mRNA sequence comprising a NSAE exon. In some embodiments, the ASO targets a WRAP53 pre-mRNA sequence comprising an intron flanking the 3′ splice site of the NSAE exon. In some embodiments, the ASO targets a WRAP53 pre-mRNA sequence comprising an intron flanking the 5′ splice site of the NSAE exon. In some embodiments, the transcript is selected from the group consisting of transcript_ENST00000316024.9, transcript_ENST00000396463.6, transcript_ENST00000431639.6, transcript_ENST00000457584.6, transcript_ENST00000463804.6, transcript_ENST00000467699.5, transcript_ENST00000471973.6, transcript_ENST00000498114.1, transcript_ENST00000498311.5, and transcript_ENST00000534050.5.

In some embodiments, the WRAP53 pre-mRNA transcript is encoded by a genetic sequence with at least about 80%, 85%, 90%, 95%, 96%, 97%, 98%, 99% or 100% sequence identity to the Ensembl reference number ENSG00000141499.17 or a complement thereof. In some embodiments, the WRAP53 pre-mRNA transcript comprises a sequence with at least about 80%, 85%, 90%, 95%, 96%, 97%, 98%, 99% or 100% sequence identity to a WRAP53 pre-mRNA transcript or a complement thereof described herein.

In some embodiments, the targeted portion of the WRAP53 pre-mRNA comprises a sequence with at least 80%, 85%, 90%, 95%, 97%, or 100% sequence identity to a region comprising at least 8 contiguous nucleic acids of a sequence of premrna_ENST00000316024.9, premrna_ENST00000396463.6, premrna_ENST00000431639.6, premrna_ENST00000457584.6, premrna_ENST00000463804.6, premrna_ENST00000467699.5, premrna_ENST00000471973.6, premrna_ENST00000498114.1, premrna_ENST00000498311.5, premrna_ENST00000534050.5 or a sequence of Table 3 or complements thereof. In some embodiments, the targeted portion of the WRAP53 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1078-SEQ ID NO: 1081 or complements thereof. In some embodiments, the targeted portion of the WRAP53 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a XPO1 genomic sequence. In some embodiments, the ASO targets a NSAE pre-mRNA transcript from a XPO1 genomic sequence comprising a NSAE exon. In some embodiments, the ASO targets a NSAE pre-mRNA transcript from a XPO1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000401558.7, premrna_ENST00000404992.6, premrna_ENST00000406957.5, premrna_ENST00000420673.5, premrna_ENST00000422552.5, premrna_ENST00000428210.5, premrna_ENST00000436018.1, premrna_ENST00000437159.5, premrna_ENST00000443240.5, premrna_ENST00000449444.5, premrna_ENST00000451765.5, premrna_ENST00000457483.5, premrna_ENST00000460037.1, premrna_ENST00000461407.5, premrna_ENST00000468259.1, premrna_ENST00000469337.1, premrna_ENST00000475744.1, premrna_ENST00000476585.5, premrna_ENST00000481073.5, premrna_ENST00000481214.1, premrna_ENST00000489954.1, premrna_ENST00000492182.5, premrna_ENST00000494468.1, and premrna_ENST00000495003.1. In some embodiments, the ASO targets a XPO1 pre-mRNA sequence comprising a NSAE exon. In some embodiments, the ASO targets a XPO1 pre-mRNA sequence comprising an intron flanking the 3′ splice site of the NSAE exon. In some embodiments, the ASO targets a XPO1 pre-mRNA sequence comprising an intron flanking the 5′ splice site of the NSAE exon. In some embodiments, the transcript is selected from the group consisting of transcript_ENST00000401558.7, transcript_ENST00000404992.6, transcript_ENST00000406957.5, transcript_ENST00000420673.5, transcript_ENST00000422552.5, transcript_ENST00000428210.5, transcript_ENST00000436018.1, transcript_ENST00000437159.5, transcript_ENST00000443240.5, transcript_ENST00000449444.5, transcript_ENST00000451765.5, transcript_ENST00000457483.5, transcript_ENST00000460037.1, transcript_ENST00000461407.5, transcript_ENST00000468259.1, transcript_ENST00000469337.1, transcript_ENST00000475744.1, transcript_ENST00000476585.5, transcript_ENST00000481073.5, transcript_ENST00000481214.1, transcript_ENST00000489954.1, transcript_ENST00000492182.5, transcript_ENST00000494468.1, and transcript_ENST00000495003.1.

In some embodiments, the XPO1 pre-mRNA transcript is encoded by a genetic sequence with at least about 80%, 85%, 90%, 95%, 96%, 97%, 98%, 99% or 100% sequence identity to the Ensembl reference number ENSG00000082898.17 or a complement thereof. In some embodiments, the XPO1 pre-mRNA transcript comprises a sequence with at least about 80%, 85%, 90%, 95%, 96%, 97%, 98%, 99% or 100% sequence identity to a XPO1 pre-mRNA transcript or a complement thereof described herein.

In some embodiments, the targeted portion of the XPO1 pre-mRNA comprises a sequence with at least 80%, 85%, 90%, 95%, 97%, or 100% sequence identity to a region comprising at least 8 contiguous nucleic acids of a sequence of premrna_ENST00000401558.7, premrna_ENST00000404992.6, premrna_ENST00000406957.5, premrna_ENST00000420673.5, premrna_ENST00000422552.5, premrna_ENST00000428210.5, premrna_ENST00000436018.1, premrna_ENST00000437159.5, premrna_ENST00000443240.5, premrna_ENST00000449444.5, premrna_ENST00000451765.5, premrna_ENST00000457483.5, premrna_ENST00000460037.1, premrna_ENST00000461407.5, premrna_ENST00000468259.1, premrna_ENST00000469337.1, premrna_ENST00000475744.1, premrna_ENST00000476585.5, premrna_ENST00000481073.5, premrna_ENST00000481214.1, premrna_ENST00000489954.1, premrna_ENST00000492182.5, premrna_ENST00000494468.1, premrna_ENST00000495003.1 or a sequence of Table 3 or complements thereof. In some embodiments, the targeted portion of the XPO1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1082-SEQ ID NO: 1085 or complements thereof. In some embodiments, the targeted portion of the XPO1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a YY1AP1 genomic sequence. In some embodiments, the ASO targets a NSAE pre-mRNA transcript from a YY1AP1 genomic sequence comprising a NSAE exon. In some embodiments, the ASO targets a NSAE pre-mRNA transcript from a YY1AP1 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000295566.8, premrna_ENST00000311573.9, premrna_ENST00000347088.9, premrna_ENST00000354691.9, premrna_ENST00000355499.8, premrna_ENST00000359205.9, premrna_ENST00000361140.8, premrna_ENST00000361831.9, premrna_ENST00000368330.6, premrna_ENST00000368339.9, premrna_ENST00000368340.9, premrna_ENST00000404643.5, premrna_ENST00000405763.7, premrna_ENST00000407221.5, premrna_ENST00000436865.5, premrna_ENST00000442834.6, premrna_ENST00000443231.5, premrna_ENST00000454523.5, premrna_ENST00000466366.1, premrna_ENST00000476027.5, premrna_ENST00000476093.5, premrna_ENST00000477470.1, premrna_ENST00000488784.5, premrna_ENST00000493625.5, and premrna_ENST00000496324.5. In some embodiments, the ASO targets a YY1AP1 pre-mRNA sequence comprising a NSAE exon. In some embodiments, the ASO targets a YY1AP1 pre-mRNA sequence comprising an intron flanking the 3′ splice site of the NSAE exon. In some embodiments, the ASO targets a YY1AP1 pre-mRNA sequence comprising an intron flanking the 5′ splice site of the NSAE exon. In some embodiments, the transcript is selected from the group consisting of transcript_ENST00000295566.8, transcript_ENST00000311573.9, transcript_ENST00000347088.9, transcript_ENST00000354691.9, transcript_ENST00000355499.8, transcript_ENST00000359205.9, transcript_ENST00000361140.8, transcript_ENST00000361831.9, transcript_ENST00000368330.6, transcript_ENST00000368339.9, transcript_ENST00000368340.9, transcript_ENST00000404643.5, transcript_ENST00000405763.7, transcript_ENST00000407221.5, transcript_ENST00000436865.5, transcript_ENST00000442834.6, transcript_ENST00000443231.5, transcript_ENST00000454523.5, transcript_ENST00000466366.1, transcript_ENST00000476027.5, transcript_ENST00000476093.5, transcript_ENST00000477470.1, transcript_ENST00000488784.5, transcript_ENST00000493625.5, and transcript_ENST00000496324.5.

In some embodiments, the YY1AP1 pre-mRNA transcript is encoded by a genetic sequence with at least about 80%, 85%, 90%, 95%, 96%, 97%, 98%, 99% or 100% sequence identity to the Ensembl reference number ENSG00000163374.19 or a complement thereof. In some embodiments, the YY1AP1 pre-mRNA transcript comprises a sequence with at least about 80%, 85%, 90%, 95%, 96%, 97%, 98%, 99% or 100% sequence identity to a YY1AP1 pre-mRNA transcript or a complement thereof described herein.

In some embodiments, the targeted portion of the YY1AP1 pre-mRNA comprises a sequence with at least 80%, 85%, 90%, 95%, 97%, or 100% sequence identity to a region comprising at least 8 contiguous nucleic acids of a sequence of premrna_ENST00000295566.8, premrna_ENST00000311573.9, premrna_ENST00000347088.9, premrna_ENST00000354691.9, premrna_ENST00000355499.8, premrna_ENST00000359205.9, premrna_ENST00000361140.8, premrna_ENST00000361831.9, premrna_ENST00000368330.6, premrna_ENST00000368339.9, premrna_ENST00000368340.9, premrna_ENST00000404643.5, premrna_ENST00000405763.7, premrna_ENST00000407221.5, premrna_ENST00000436865.5, premrna_ENST00000442834.6, premrna_ENST00000443231.5, premrna_ENST00000454523.5, premrna_ENST00000466366.1, premrna_ENST00000476027.5, premrna_ENST00000476093.5, premrna_ENST00000477470.1, premrna_ENST00000488784.5, premrna_ENST00000493625.5, premrna_ENST00000496324.5 or a sequence of Table 3 or complements thereof. In some embodiments, the targeted portion of the YY1AP1 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1086-SEQ ID NO: 1089 or complements thereof. In some embodiments, the targeted portion of the YY1AP1 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a ZC3H14 genomic sequence. In some embodiments, the ASO targets a NSAE pre-mRNA transcript from a ZC3H14 genomic sequence comprising a NSAE exon. In some embodiments, the ASO targets a NSAE pre-mRNA transcript from a ZC3H14 genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the group consisting of premrna_ENST00000251038.10, premrna_ENST00000302216.12, premrna_ENST00000318308.10, premrna_ENST00000336693.8, premrna_ENST00000393514.9, premrna_ENST00000406216.7, premrna_ENST00000553495.5, premrna_ENST00000554020.5, premrna_ENST00000554602.5, premrna_ENST00000555120.1, premrna_ENST00000555755.5, premrna_ENST00000555792.1, premrna_ENST00000555799.5, premrna_ENST00000555851.6, premrna_ENST00000555900.5, premrna_ENST00000556000.5, premrna_ENST00000556110.5, premrna_ENST00000556158.5, premrna_ENST00000556945.5, premrna_ENST00000557047.2, premrna_ENST00000557491.1, premrna_ENST00000557605.1, premrna_ENST00000557607.5, premrna_ENST00000557693.5, premrna_ENST00000557737.1, and premrna_ENST00000649731.1. In some embodiments, the ASO targets a ZC3H14 pre-mRNA sequence comprising a NSAE exon. In some embodiments, the ASO targets a ZC3H14 pre-mRNA sequence comprising an intron flanking the 3′ splice site of the NSAE exon. In some embodiments, the ASO targets a ZC3H14 pre-mRNA sequence comprising an intron flanking the 5′ splice site of the NSAE exon. In some embodiments, the transcript is selected from the group consisting of transcript_ENST00000251038.10, transcript_ENST00000302216.12, transcript_ENST00000318308.10, transcript_ENST00000336693.8, transcript_ENST00000393514.9, transcript_ENST00000406216.7, transcript_ENST00000553495.5, transcript_ENST00000554020.5, transcript_ENST00000554602.5, transcript_ENST00000555120.1, transcript_ENST00000555755.5, transcript_ENST00000555792.1, transcript_ENST00000555799.5, transcript_ENST00000555851.6, transcript_ENST00000555900.5, transcript_ENST00000556000.5, transcript_ENST00000556110.5, transcript_ENST00000556158.5, transcript_ENST00000556945.5, transcript_ENST00000557047.2, transcript_ENST00000557491.1, transcript_ENST00000557605.1, transcript_ENST00000557607.5, transcript_ENST00000557693.5, transcript_ENST00000557737.1, and transcript_ENST00000649731.1.

In some embodiments, the ZC3H14 pre-mRNA transcript is encoded by a genetic sequence with at least about 80%, 85%, 90%, 95%, 96%, 97%, 98%, 99% or 100% sequence identity to the Ensembl reference number ENSG00000100722.20 or a complement thereof. In some embodiments, the ZC3H14 pre-mRNA transcript comprises a sequence with at least about 80%, 85%, 90%, 95%, 96%, 97%, 98%, 99% or 100% sequence identity to a ZC3H14 pre-mRNA transcript or a complement thereof described herein.

In some embodiments, the targeted portion of the ZC3H14 pre-mRNA comprises a sequence with at least 80%, 85%, 90%, 95%, 97%, or 100% sequence identity to a region comprising at least 8 contiguous nucleic acids of a sequence of premrna_ENST00000251038.10, premrna_ENST00000302216.12, premrna_ENST00000318308.10, premrna_ENST00000336693.8, premrna_ENST00000393514.9, premrna_ENST00000406216.7, premrna_ENST00000553495.5, premrna_ENST00000554020.5, premrna_ENST00000554602.5, premrna_ENST00000555120.1, premrna_ENST00000555755.5, premrna_ENST00000555792.1, premrna_ENST00000555799.5, premrna_ENST00000555851.6, premrna_ENST00000555900.5, premrna_ENST00000556000.5, premrna_ENST00000556110.5, premrna_ENST00000556158.5, premrna_ENST00000556945.5, premrna_ENST00000557047.2, premrna_ENST00000557491.1, premrna_ENST00000557605.1, premrna_ENST00000557607.5, premrna_ENST00000557693.5, premrna_ENST00000557737.1, premrna_ENST00000649731.1 or a sequence of Table 3 or complements thereof. In some embodiments, the targeted portion of the ZC3H14 pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 1090-SEQ ID NO: 1098 or complements thereof. In some embodiments, the targeted portion of the ZC3H14 pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASOs disclosed herein target a NSAE pre-mRNA transcribed from a target genomic sequence. In some embodiments, the ASO targets a NSAE pre-mRNA transcript from a target genomic sequence comprising a NSAE exon. In some embodiments, the ASO targets a NSAE pre-mRNA transcript from a target genomic sequence comprising an intron flanking the 3′ splice site of the NSAE exon and an intron flanking the 5′ splice site of a NSAE exon. In some embodiments, the ASO targets a NSAE pre-mRNA transcript comprising a sequence selected from the pre-mRNA transcript sequences of Table 4. In some embodiments, the ASO targets a NSAE pre-mRNA transcript comprising a sequence selected from the pre-mRNA transcript sequences of Table 4 as represented by the Ensembl reference numbers. In some embodiments, the ASO targets a target pre-mRNA sequence comprising a NSAE exon. In some embodiments, the ASO targets a target pre-mRNA sequence comprising an intron flanking the 3′ splice site of the NSAE exon. In some embodiments, the ASO targets a target pre-mRNA sequence comprising an intron flanking the 5′ splice site of the NSAE exon. In some embodiments, the transcript is selected from the group consisting of the transcript sequences of Table 4. In some embodiments, the transcript is selected from the group consisting of the transcript sequences of Table 4 as represented by the Ensembl reference numbers.

In some embodiments, the target pre-mRNA transcript is encoded by a genetic sequence with at least about 80%, 85%, 90%, 95%, 96%, 97%, 98%, 99% or 100% sequence identity to the gene sequence as represented by the Ensembl reference number or a complement thereof. In some embodiments, the target pre-mRNA transcript comprises a sequence with at least about 80%, 85%, 90%, 95%, 96%, 97%, 98%, 99% or 100% sequence identity to target pre-mRNA transcript or a complement thereof described herein.

In some embodiments, the targeted portion of the target pre-mRNA comprises a sequence with at least 80%, 85%, 90%, 95%, 97%, or 100% sequence identity to a region comprising at least 8 contiguous nucleic acids of a sequence of Table 4 or a sequence of Table 3 or complements thereof. In some embodiments, the targeted portion of the target pre-mRNA comprises a sequence with at least 80%, 85%, 90%, 95%, 97%, or 100% sequence identity to a region comprising at least 8 contiguous nucleic acids of a sequence of Table 4 as represented by the Ensembl reference numbers or a sequence of Table 3 or complements thereof. In some embodiments, the targeted portion of the target pre-mRNA comprises a sequence with at least 80%, 85%, 90%, 95%, 97%, or 100% sequence identity to a region comprising at least 8 contiguous nucleic acids of a sequence selected from the group consisting of SEQ ID NO: 170-SEQ ID NO: 1098 or complements thereof. In some embodiments, the targeted portion of the target pre-mRNA comprises a sequence that is complementary to at least 8, 9, 10, 11, 12, 13, 14, 15, 16, 17, 18, 19 or 20 contiguous nucleic acids of a sequence of Table 6A or Table 6B or a complement thereof.

In some embodiments, the ASO targets exon 11 of a SYNGAP1 pre-mRNA comprising a NSAE exon. In some embodiments, the ASO targets a sequence about 2 nucleotides downstream (or 3′) from the 3′ splice site of exon 11 to about 4 nucleotides upstream (or 5′) from the 5′ splice site of exon 11. In some embodiments, the ASO has a sequence according to any one of SEQ ID NOs: _or complements thereof.

In some embodiments, the ASO targets an exon of a target pre-mRNA comprising a NSAE exon, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the ASO targets a sequence about 2 nucleotides downstream (or 3′) from the 3′ splice site of the exon to about 4 nucleotides upstream (or 5′) from the 5′ splice site of the exon. In some embodiments, the ASO has a sequence according to any one of SEQ ID NOs: _ or complements thereof.

In some embodiments, the ASO targets intron 10 of a SYNGAP1 pre-mRNA comprising a NSAE exon. In some embodiments, the ASO targets a sequence about 4 to about 300 nucleotides upstream (or 5′) from the 3′ splice site of exon 11. In some embodiments, the ASO targets a sequence about 16 to about 100 nucleotides upstream (or 5′) from the 3′ splice site of exon 11. In some embodiments, the ASO targets a sequence about 4 to about 300 nucleotides downstream (or 3′) from the 5′ splice site of exon 10. In some embodiments, the ASO has a sequence according to any sequence of Table 1 or complements thereof.

In some embodiments, the ASO targets an intron of a target pre-mRNA comprising a NSAE exon, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the ASO targets a sequence about 4 to about 300 nucleotides upstream (or 5′) from the 3′ splice site of the exon. In some embodiments, the ASO targets a sequence about 16 to about 100 nucleotides upstream (or 5′) from the 3′ splice site of the exon. In some embodiments, the ASO targets a sequence about 4 to about 300 nucleotides downstream (or 3′) from the 5′ splice site of the exon. In some embodiments, the ASO has a sequence according to any sequence of Table 1 or complements thereof.

In some embodiments, the ASO targets intron 10 of a SYNGAP1 pre-mRNA comprising a NSAE exon. In some embodiments, the ASO targets a sequence about 4 to about 300 nucleotides upstream (or 5′) from the 3′ splice site of exon 11. In some embodiments, the ASO targets a sequence about 4 to about 300 nucleotides downstream (or 3′) from the 5′ splice site of intron 10. In some embodiments, the ASO targets a sequence about 6 to about 100 nucleotides downstream (or 5′) from the 5′ splice site of intron 10. In some embodiments, the ASO has a sequence according to any sequence of Table 1 or complements thereof.

In some embodiments, the ASO targets an intron of a pre-mRNA comprising a NSAE exon, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the ASO targets a sequence about 4 to about 300 nucleotides upstream (or 5′) from the 3′ splice site of the exon. In some embodiments, the ASO targets a sequence about 4 to about 300 nucleotides downstream (or 3′) from the 5′ splice site of the intron. In some embodiments, the ASO targets a sequence about 6 to about 100 nucleotides downstream (or 5′) from the 5′ splice site of the intron. In some embodiments, the ASO has a sequence according to any sequence of Table 1 or complements thereof.

In some embodiments, the targeted portion of the SYNGAP1 pre-mRNA is in intron 9, 10, 11 or 12 In some embodiments, the targeted portion of the SYNGAP1 pre-mRNA is in exon 9, 10, 11 or 12. In some embodiments, hybridization of an ASO to the targeted portion of the NSAE pre-mRNA results in inclusion of canonical exon 11, and subsequently increases SYNGAP1 protein production. In some embodiments, hybridization of an ASO to the targeted portion of the NSAE pre-mRNA results in exclusion of a canonical exon, and subsequently decreases SYNGAP1 protein production. In some embodiments, hybridization of an ASO to the targeted portion of the NSAE pre-mRNA results in inclusion or exclusion of a canonical exon, and subsequently modulates SYNGAP1 protein production. In some embodiments, the targeted portion of the SYNGAP1 pre-mRNA is in exon 10 or 11. In some embodiments, the targeted portion of the SYNGAP1 pre-mRNA is in intron 10. In some embodiments, the targeted portion of the SYNGAP1 pre-mRNA is in intron 11.

In some embodiments, the targeted portion of the target pre-mRNA is in an intron, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the targeted portion of the target pre-mRNA is in an exon. In some embodiments, hybridization of an ASO to the targeted portion of the NSAE pre-mRNA results in inclusion of a canonical exon, and subsequently increases target protein production. In some embodiments, hybridization of an ASO to the targeted portion of the NSAE pre-mRNA results in exclusion of a canonical exon, and subsequently decreases target protein production. In some embodiments, hybridization of an ASO to the targeted portion of the NSAE pre-mRNA results in inclusion or exclusion of a canonical exon, and subsequently modulates target protein production.

### SYNGAP1 Protein

In some embodiments, a mutation occurs in both alleles. In some embodiments, a mutation occurs in one of the two alleles. In some embodiments, additional mutation occurs in one of the two alleles. In some embodiments, the additional mutation occurs in the same allele as the first mutation. In other embodiments, the additional mutation occurs is a trans mutation.

In some embodiments, the methods described herein are used to increase the production of a functional SYNGAP1 protein. In some embodiments, the methods described herein are used to decrease the production of a functional SYNGAP1 protein. In some embodiments, the methods described herein are used to modulate the production of a functional SYNGAP1 protein. As used herein, the term “functional” refers to the amount of activity or function of a SYNGAP1 protein that is necessary to prevent or eliminate any one or more symptoms of a treated disease or condition, e.g., autosomal dominant mental retardation. Alternatively, overexpression of a functional SYNGAP1 protein may induce or increase any one or more symptoms of a treated disease or condition. In some embodiments, the methods are used to increase the production of a partially functional SYNGAP1 protein. In some embodiments, the methods are used to decrease the production of a partially functional SYNGAP1 protein. In some embodiments, the methods are used to modulate the production of a partially functional SYNGAP1 protein. As used herein, the term “partially functional” refers to any amount of activity or function of the SYNGAP1 protein that is less than the amount of activity or function that is necessary to eliminate or prevent any one or more symptoms of a disease or condition, e.g., autosomal dominant mental retardation. Alternatively, expression or overexpression of a functional SYNGAP1 protein may induce or increase any one or more symptoms of a treated disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the SYNGAP1 protein by cells of a subject having a NSAE pre-mRNA encoding the SYNGAP1 protein, wherein the subject has autosomal dominant mental retardation caused by a deficient amount of activity of SYNGAP1 protein, and wherein the deficient amount of the SYNGAP1 protein is caused by sporadic mutation. In such an embodiment, the subject has a first allele carrying the_mutation and a second allele from which the SYNGAP1 protein is not produced. In another such embodiment, the subject has a first allele carrying a mutation and a second allele encoding a nonfunctional SYNGAP1 protein. In another such embodiment, the subject has a first allele carrying a mutation and a second allele encoding a partially functional SYNGAP1 protein. In another such an embodiment, the subject has a first allele carrying a mutation and a second allele carrying the mutation. In any of these embodiments, the antisense oligomer binds to a targeted portion of the NSAE pre-mRNA transcribed from the allele carrying a mutation, thereby prevent alternate splicing of NSAEs into the pre-mRNA, and causing an increase in the level of mature mRNA encoding functional SYNGAP1 protein, and an increase in the expression of the SYNGAP1 protein in the cells of the subject.

In related embodiments, the method is a method of using an ASO to increase the expression of a functional protein or functional RNA. In some embodiments, an ASO is used to increase the expression of SYNGAP1 protein in cells of a subject having a NSAE pre-mRNA encoding SYNGAP1 protein, wherein the subject has a deficiency, e.g., autosomal dominant mental retardation, in the amount or function of SYNGAP1 protein.

In some embodiments, the method is a method of decreasing the expression of the SYNGAP1 protein by cells of a subject having a NSAE pre-mRNA encoding the SYNGAP1 protein, wherein the subject has a disease caused by an excess amount of activity of SYNGAP1 protein, and wherein the excess amount of SYNGAP1 protein is caused by a mutation. In some embodiments, the antisense oligomer binds to a targeted portion of the NSAE pre-mRNA transcribed from the allele carrying a mutation, thereby increasing alternate splicing of NSAEs into the pre-mRNA, and causing an decrease in the level of mature mRNA encoding functional SYNGAP1 protein, and an decrease in the expression of the SYNGAP1 protein in the cells of the subject. In related embodiments, the method is a method of using an ASO to decrease the expression of a functional protein or functional RNA. In some embodiments, an ASO is used to decrease the expression of SYNGAP1 protein in cells of a subject having a NSAE pre-mRNA encoding the SYNGAP1 protein, wherein the subject has an excess in the amount or function of SYNGAP1 protein.

In some embodiments, the method is a method of modulating the expression of the SYNGAP1 protein by cells of a subject having a NSAE pre-mRNA encoding the SYNGAP1 protein, wherein the subject has a disease caused by a deficient or excess amount of activity of SYNGAP1 protein, and wherein the deficient or excess amount of the SYNGAP1 protein is caused by a mutation. In some embodiments, the antisense oligomer binds to a targeted portion of the NSAE pre-mRNA transcribed from the allele carrying a mutation, thereby modulating alternate splicing of NSAEs into the pre-mRNA, and causing an modulation in the level of mature mRNA encoding functional SYNGAP1 protein, and an modulation in the expression of SYNGAP1 protein in the cells of the subject. In related embodiments, the method is a method of using an ASO to modulate the expression of a functional protein or functional RNA. In some embodiments, an ASO is used to modulate the expression of SYNGAP1 protein in cells of a subject having a NSAE pre-mRNA encoding the SYNGAP1 protein, wherein the subject has an abnormality in the amount or function of the SYNGAP1 protein.

In some embodiments, the NSAE pre-mRNA transcript that encodes the protein that is causative of the disease or condition is targeted by the ASOs described herein. In some embodiments, a NSAE pre-mRNA transcript that encodes a protein that is not causative of the disease is targeted by the ASOs. For example, a disease that is the result of a mutation or deficiency of a first protein in a particular pathway may be ameliorated by targeting a NSAE containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele carrying a mutation from which the SYNGAP1 protein is produced at a reduced level compared to production from a wild-type allele, and
(b) a second mutant allele from which
   (i) the SYNGAP1 protein is produced at a reduced level compared to production from a wild-type allele due to the_mutation
   (ii) the SYNGAP1 protein is produced at a reduced level compared to production from a wild-type allele,
   (iii) the SYNGAP1 protein is produced in a form having reduced function compared to an equivalent wild-type protein, or
   (iv) the SYNGAP1 protein is not produced, and
wherein the NSAE pre-mRNA is transcribed from the first allele and/or the second allele carrying the mutation. In these embodiments, the ASO binds to a targeted portion of the NSAE pre-mRNA transcribed from the first allele or the second allele, thereby promoting exon inclusion from the NSAE pre-mRNA, and causing an increase in the level of full-length mRNA encoding SYNGAP1 protein and an increase in the expression of the target protein or functional RNA in the cells of the subject. In these embodiments, the target protein or functional RNA having an increase in expression level resulting from exon inclusion from the NSAE pre-mRNA has full function compared to the equivalent wild-type protein (fully-functional). In these embodiments, the ASO binds to a targeted portion of the NSAE pre-mRNA transcribed from the first allele or the second allele, thereby reducing exon inclusion from the NSAE pre-mRNA, and causing a decrease in the level of full-length mRNA encoding SYNGAP1 protein and a decrease in the expression of the target protein or functional RNA in the cells of the subject. In these embodiments, the target protein or functional RNA having a decrease in expression level resulting from exon exclusion from the NSAE pre-mRNA has full function compared to the equivalent wild-type protein (fully-functional).

In some embodiments, contacting cells with an ASO that is complementary to a targeted portion of a SYNGAP1 pre-mRNA transcript results in an increase in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SYNGAP1 protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding SYNGAP1 protein is increased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 containing pre-mRNA.

In some embodiments, the level of mRNA encoding SYNGAP1 protein is increased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 pre-mRNA.

In some embodiments of the present invention, a subject can have a mutation in SYNGAP1. A variety of pathogenic variants have been reported to cause SYNGAP1 deficiency, including missense variants, nonsense variants, single- and double-nucleotide insertions and deletions, complex insertion/deletions, and splice site variants. In the presence of this pathogenic variant approximately 2%-5% of transcripts are correctly spliced, allowing for residual enzyme activity. In some embodiments, disease results from loss of function of SYNGAP1 caused by SYNGAP1 pathogenic variants that generate truncated proteins or proteins with altered conformations or reduced activity.

In some embodiments, a subject having any SYNGAP1 mutation known in the art and described as above can be treated using the methods and compositions described herein. In some embodiments, the mutation is within any SYNGAP1 intron or exon. In some embodiments, the mutation is within SYNGAP1 exon 10 or 11.

In some embodiments, contacting cells with an ASO that is complementary to a targeted portion of a SYNGAP1 pre-mRNA transcript results in a decrease in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SYNGAP1 protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding SYNGAP1 protein is decreased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 containing pre-mRNA.

In some embodiments, the level of mRNA encoding SYNGAP1 protein is decreased 1.1 to 10-fold, when compared to the amount of mRNA encoding SYNGAP1 protein that is produced in a control cell, e.g., one that is not treated with the antisense oligomer or one that is treated with an antisense oligomer that does not bind to the targeted portion of the SYNGAP1 pre-mRNA.

In some embodiments of the present invention, a subject can have a mutation in SYNGAP1. A variety of pathogenic variants may cause SYNGAP1 overexpression, including missense variants, nonsense variants, single- and double-nucleotide insertions and deletions, complex insertion/deletions, and splice site variants. In some embodiments, disease results from an overexpression of function of SYNGAP1 caused by SYNGAP1 pathogenic variants that generate truncated proteins or proteins with altered conformations or increased activity.

In some embodiments, a subject having any SYNGAP1 mutation known in the art and described as above can be treated using the methods and compositions described herein. In some embodiments, the mutation is within any SYNGAP1 intron or exon. In some embodiments, the mutation is within SYNGAP1 exon 10 or 11.

### Target Protein

In some embodiments, a mutation occurs in both alleles. In some embodiments, a mutation occurs in one of the two alleles, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, additional mutation occurs in one of the two alleles. In some embodiments, the additional mutation occurs in the same allele as the first mutation. In other embodiments, the additional mutation occurs is a trans mutation.

In some embodiments, the methods described herein are used to increase the production of a functional target protein, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the methods described herein are used to decrease the production of a functional target protein. In some embodiments, the methods described herein are used to modulate the production of a functional target protein. As used herein, the term “functional” refers to the amount of activity or function of a target protein that is necessary to prevent or eliminate any one or more symptoms of a treated disease or condition, e.g., autosomal dominant mental retardation. Alternatively, overexpression of a functional target protein may induce or increase any one or more symptoms of a treated disease or condition. In some embodiments, the methods are used to increase the production of a partially functional target protein. In some embodiments, the methods are used to decrease the production of a partially functional target protein. In some embodiments, the methods are used to modulate the production of a partially functional target protein. As used herein, the term “partially functional” refers to any amount of activity or function of the target protein that is less than the amount of activity or function that is necessary to eliminate or prevent any one or more symptoms of a disease or condition, e.g., autosomal dominant mental retardation. Alternatively, expression or overexpression of a functional target protein may induce or increase any one or more symptoms of a treated disease or condition. In some embodiments, a partially functional protein or RNA will have at least 10%, at least 20%, at least 30%, at least 40%, at least 50%, at least 60%, at least 70%, at least 75%, at least 80%, at least 85%, at least 90%, or at least 95% less activity relative to the fully functional protein or RNA.

In some embodiments, the method is a method of increasing the expression of the target protein by cells of a subject having a NSAE pre-mRNA encoding the target protein, wherein the subject has autosomal dominant mental retardation caused by a deficient amount of activity of target protein, wherein the deficient amount of the target protein is caused by sporadic mutation, and wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In such an embodiment, the subject has a first allele carrying a mutation and a second allele from which the target protein is not produced. In another such embodiment, the subject has a first allele carrying a mutation and a second allele encoding a nonfunctional target protein. In another such embodiment, the subject has a first allele carrying a mutation and a second allele encoding a partially functional target protein. In another such an embodiment, the subject has a first allele carrying a mutation and a second allele carrying the mutation. In any of these embodiments, the antisense oligomer binds to a targeted portion of the NSAE pre-mRNA transcribed from the allele carrying a mutation, thereby prevent alternate splicing of NSAEs into the pre-mRNA, and causing an increase in the level of mature mRNA encoding functional target protein, and an increase in the expression of the target protein in the cells of the subject. In related embodiments, the method is a method of using an ASO to increase the expression of a functional protein or functional RNA. In some embodiments, an ASO is used to increase the expression of target protein in cells of a subject having a NSAE pre-mRNA encoding the target protein, wherein the subject has a deficiency, e.g., autosomal dominant mental retardation, in the amount or function of the target protein.

In some embodiments, the method is a method of decreasing the expression of the target protein by cells of a subject having a NSAE pre-mRNA encoding the target protein, wherein the subject has a disease caused by an excess amount of activity of the target protein, wherein the excess amount of the target protein is caused by a mutation, and wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the antisense oligomer binds to a targeted portion of the NSAE pre-mRNA transcribed from the allele carrying a mutation, thereby increasing alternate splicing of NSAEs into the pre-mRNA, and causing an decrease in the level of mature mRNA encoding the functional target protein, and an decrease in the expression of the target protein in the cells of the subject. In related embodiments, the method is a method of using an ASO to decrease the expression of a functional protein or functional RNA. In some embodiments, an ASO is used to decrease the expression of the target protein in cells of a subject having a NSAE pre-mRNA encoding the target protein, wherein the subject has an excess in the amount or function of the target protein.

In some embodiments, the method is a method of modulating the expression of the target protein by cells of a subject having a NSAE pre-mRNA encoding the target protein, wherein the subject has a disease caused by a deficient or excess amount of activity of the target protein, wherein the deficient or excess amount of the target protein is caused by a mutation, and wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the antisense oligomer binds to a targeted portion of the NSAE pre-mRNA transcribed from the allele carrying a mutation, thereby modulating alternate splicing of NSAEs into the pre-mRNA, and causing an modulation in the level of mature mRNA encoding the functional target protein, and an modulation in the expression of the target protein in the cells of the subject. In related embodiments, the method is a method of using an ASO to modulate the expression of a functional protein or functional RNA. In some embodiments, an ASO is used to modulate the expression of the target protein in cells of a subject having a NSAE pre-mRNA encoding the target protein, wherein the subject has an abnormality in the amount or function of the target protein.

In some embodiments, the NSAE pre-mRNA transcript that encodes the protein that is causative of the disease or condition is targeted by the ASOs described herein. In some embodiments, a NSAE pre-mRNA transcript that encodes a protein that is not causative of the disease is targeted by the ASOs. For example, a disease that is the result of a mutation or deficiency of a first protein in a particular pathway may be ameliorated by targeting a NSAE containing pre-mRNA that encodes a second protein, thereby increasing production of the second protein. In some embodiments, the function of the second protein is able to compensate for the mutation or deficiency of the first protein (which is causative of the disease or condition).

In some embodiments, the subject has:
(a) a first mutant allele carrying a mutation from which the target protein is produced at a reduced level compared to production from a wild-type allele, and
(c) a second mutant allele from which
   (v) the target protein is produced at a reduced level compared to production from a wild-type allele due to the_mutation
   (vi) the target protein is produced at a reduced level compared to production from a wild-type allele,
   (vii) the target protein is produced in a form having reduced function compared to an equivalent wild-type protein, or
   (viii) the target protein is not produced, and
wherein the NSAE pre-mRNA is transcribed from the first allele and/or the second allele carrying the mutation and wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In these embodiments, the ASO binds to a targeted portion of the NSAE pre-mRNA transcribed from the first allele or the second allele, thereby promoting exon inclusion from the NSAE pre-mRNA, and causing an increase in the level of full-length mRNA encoding the target protein and an increase in the expression of the target protein or functional RNA in the cells of the subject. In these embodiments, the target protein or functional RNA having an increase in expression level resulting from exon inclusion from the NSAE pre-mRNA has full function compared to the equivalent wild-type protein (fully-functional). In these embodiments, the ASO binds to a targeted portion of the NSAE pre-mRNA transcribed from the first allele or the second allele, thereby reducing exon inclusion from the NSAE pre-mRNA, and causing a decrease in the level of full-length mRNA encoding the target protein and a decrease in the expression of the target protein or functional RNA in the cells of the subject. In these embodiments, the target protein or functional RNA having a decrease in expression level resulting from exon exclusion from the NSAE pre-mRNA has full function compared to the equivalent wild-type protein (fully-functional).

In some embodiments, contacting cells with an ASO that is complementary to a targeted portion of a target pre-mRNA transcript results in an increase in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the total amount of the target protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding the target protein is increased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target containing pre-mRNA, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the level of mRNA encoding the target protein is increased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target pre-mRNA.

In some embodiments of the present invention, a subject can have a mutation in the target gene, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. A variety of pathogenic variants have been reported to cause the target deficiency, including missense variants, nonsense variants, single- and double-nucleotide insertions and deletions, complex insertion/deletions, and splice site variants. In some embodiments, disease results from loss of function of the target caused by the target pathogenic variants that generate truncated proteins or proteins with altered conformations or reduced activity.

In some embodiments, a subject having any mutation in the target gene known in the art and described as above can be treated using the methods and compositions described herein, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the mutation is within any intron or exon in the target gene.

In some embodiments, contacting cells with an ASO that is complementary to a targeted portion of a target pre-mRNA transcript results in a decrease in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the total amount of the target protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, the level of mRNA encoding the target protein is decreased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target containing pre-mRNA, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14.

In some embodiments, the level of mRNA encoding the target protein is decreased 1.1 to 10-fold, when compared to the amount of mRNA encoding the target protein that is produced in a control cell, e.g., one that is not treated with the antisense oligomer or one that is treated with an antisense oligomer that does not bind to the targeted portion of the target pre-mRNA, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14.

In some embodiments of the present invention, a subject can have a mutation in the target gene, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. A variety of pathogenic variants may cause the target protein overexpression, including missense variants, nonsense variants, single- and double-nucleotide insertions and deletions, complex insertion/deletions, and splice site variants. In some embodiments, disease results from an overexpression of function of the target protein caused by the pathogenic variants in the target that generate truncated proteins or proteins with altered conformations or increased activity.

In some embodiments, a subject having any mutation in the target gene known in the art and described as above can be treated using the methods and compositions described herein, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the mutation is within any intron or exon in the target gene.

### Alternative Splicing

As used herein, a “non-sense mediated RNA decay alternative exon” (or “NSAE” or “NMD exon”) is an exon created from alternative splicing events that contains a premature stop codon or other sequences that facilitate degradation of the mRNA containing the of the instant exon. “NSAE pre-mRNA” is a pre-mRNA transcript that contains at least one non-sense mediated RNA decay alternative exon. Alternative or aberrant splicing of the pre-mRNA can result in the inclusion of at least one NSAE in the mature mRNA transcripts. The terms “mature mRNA,” “fully processed,” and “fully-spliced mRNA,” are used interchangeably herein to describe a fully processed mRNA. Inclusion of at least one NSAE can result in non-productive mRNA. Mature NSAE-containing mRNA may sometimes lead to aberrant protein expression. The term “canonical exon” refers to the exon that does not lead to non-sense mediated decay, or default exon, or exon that is not the product of alternative splicing. The term “canonical splicing” refers to a splicing process that results in the preservation of the canonical exon in the mature RNA.

A NSAE can be created as a result of splicing out additional basepairs.

The degree of alternative splicing can be expressed as percent alternative splicing, e.g., the percentage of transcripts in which a given NSAE is included. In brief, percent alternative splicing can be calculated as the percentage of the amount of RNA transcripts with the NSAE, over the sum of the average of the amount of RNA transcripts with a NSAE plus the average of the amount of RNA transcripts with only the canonical exons.

In some embodiments, a NSAE is an exon that is identified as a NSAE based on a determination of at least about 5%, at least about 10%, at least about 15%, at least about 20%, at least about 25%, at least about 30%, at least about 35%, at least about 40%, at least about 45%, or at least about 50%, exclusion from the RNA transcript. In some embodiments, a NSAE is an exon that is identified as a NSAE based on a determination of about 5% to about 100%, about 5% to about 95%, about 5% to about 90%, about 5% to about 85%, about 5% to about 80%, about 5% to about 75%, about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 15% to about 100%, about 15% to about 95%, about 15% to about 90%, about 15% to about 85%, about 15% to about 80%, about 15% to about 75%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, or about 25% to about 35%, exclusion from the RNA transcript. ENCODE data (described by, e.g., Tilgner, et al., 2012, “Deep sequencing of subcellular RNA fractions shows splicing to be predominantly co-transcriptional in the human genome but inefficient for lncRNAs,” Genome Research 22(9):1616-25) can be used to aid in identifying NSAE's created from alternative splicing events.

In some embodiments, contacting cells with an ASO that is complementary to a targeted portion of a SYNGAP1 pre-mRNA transcript results in an increase in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of SYNGAP1 protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SYNGAP1 pre-mRNA transcript results in a decrease in the amount of SYNGAP1 protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the mutation is within any intron or exon in the target gene. In some embodiments, the total amount of SYNGAP1 protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of SYNGAP1 protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a SYNGAP1 pre-mRNA transcript results in an increase in the amount of mRNA encoding SYNGAP1, including the mature mRNA encoding the target protein. In some embodiments, the amount of mRNA encoding SYNGAP1 protein, or the mature mRNA encoding the SYNGAP1 protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SYNGAP1 protein, or the mature mRNA encoding SYNGAP1 protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding SYNGAP1 protein, or the mature mRNA encoding SYNGAP1 protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SYNGAP1 pre-mRNA.

In some embodiments, contacting cells with an ASO that is complementary to a targeted portion of a SYNGAP1 pre-mRNA transcript results in a decrease in the amount of mRNA encoding SYNGAP1, including the mature mRNA encoding the target protein. In some embodiments, the mutation is within any intron or exon in the target gene. In some embodiments, the amount of mRNA encoding SYNGAP1 protein, or the mature mRNA encoding the SYNGAP1 protein, is decreased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding SYNGAP1 protein, or the mature mRNA encoding SYNGAP1 protein produced in the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding SYNGAP1 protein, or the mature mRNA encoding SYNGAP1 protein produced in the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the SYNGAP1 pre-mRNA.

In some embodiments, contacting cells with an ASO that is complementary to a targeted portion of a target pre-mRNA transcript results in an increase in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the mutation is within any intron or exon in the target gene. In some embodiments, the total amount of the target protein produced by the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a target pre-mRNA transcript results in a decrease in the amount of the target protein produced by at least 10, 20, 30, 40, 50, 60, 80, 100, 150, 200, 250, 300, 350, 400, 450, 500, or 1000%, compared to the amount of the protein produced by a cell in the absence of the ASO/absence of treatment, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the mutation is within any intron or exon in the target gene. In some embodiments, the total amount of the target protein produced by the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of target protein produced by a control compound. In some embodiments, the total amount of the target protein produced by the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold, compared to the amount of target protein produced by a control compound. A control compound can be, for example, an oligonucleotide that is not complementary to a targeted portion of the pre-mRNA.

In some embodiments, contacting cells with an ASO that is complementary to a targeted portion of a target pre-mRNA transcript results in an increase in the amount of mRNA encoding the target protein, including the mature mRNA encoding the target protein, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the mutation is within any intron or exon in the target gene. In some embodiments, the amount of mRNA encoding the target protein, or the mature mRNA encoding the target protein, is increased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding the target protein, or the mature mRNA encoding the target protein produced in the cell to which the antisense oligomer is contacted is increased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding the target protein, or the mature mRNA encoding the target protein produced in the cell to which the antisense oligomer is contacted is increased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the target pre-mRNA.

In some embodiments, contacting cells with an ASO that is complementary to a targeted portion of a target pre-mRNA transcript results in a decrease in the amount of mRNA encoding the target protein, including the mature mRNA encoding the target protein, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the mutation is within any intron or exon in the target gene. In some embodiments, the amount of mRNA encoding the target protein, or the mature mRNA encoding the target protein, is decreased by at least 10, 20, 30, 40, 50, 60, 80, 100, 150, 200, 250, 300, 350, 400, 450, 500, or 1000%, compared to the amount of the protein produced by a cell in the absence of the ASO/absence of treatment. In some embodiments, the total amount of the mRNA encoding the target protein, or the mature mRNA encoding the target protein produced in the cell to which the antisense oligomer is contacted is decreased about 20% to about 300%, about 50% to about 300%, about 100% to about 300%, about 150% to about 300%, about 20% to about 50%, about 20% to about 100%, about 20% to about 150%, about 20% to about 200%, about 20% to about 250%, about 50% to about 100%, about 50% to about 150%, about 50% to about 200%, about 50% to about 250%, about 100% to about 150%, about 100% to about 200%, about 100% to about 250%, about 150% to about 200%, about 150% to about 250%, about 200% to about 250%, at least about 10%, at least about 20%, at least about 50%, at least about 100%, at least about 150%, at least about 200%, at least about 250%, or at least about 300%, compared to the amount of mature RNA produced in an untreated cell, e.g., an untreated cell or a cell treated with a control compound. In some embodiments, the total amount of the mRNA encoding the target protein, or the mature mRNA encoding the target protein produced in the cell to which the antisense oligomer is contacted is decreased about 1.1 to about 10-fold, about 1.5 to about 10-fold, about 2 to about 10-fold, about 3 to about 10-fold, about 4 to about 10-fold, about 1.1 to about 5-fold, about 1.1 to about 6-fold, about 1.1 to about 7-fold, about 1.1 to about 8-fold, about 1.1 to about 9-fold, about 2 to about 5-fold, about 2 to about 6-fold, about 2 to about 7-fold, about 2 to about 8-fold, about 2 to about 9-fold, about 3 to about 6-fold, about 3 to about 7-fold, about 3 to about 8-fold, about 3 to about 9-fold, about 4 to about 7-fold, about 4 to about 8-fold, about 4 to about 9-fold, at least about 1.1-fold, at least about 1.5-fold, at least about 2-fold, at least about 2.5-fold, at least about 3-fold, at least about 3.5-fold, at least about 4-fold, at least about 5-fold, or at least about 10-fold compared to the amount of mature RNA produced in an untreated cell, e.g., an untreated cell or a cell treated with a control compound. A control compound can be, for example, an oligonucleotide that is not complementary to a targeted portion of the target pre-mRNA.

### Therapeutic Agents

In various embodiments of the present disclosure, compositions and methods comprising a therapeutic agent are provided to modulate protein expression level of SYNGAP1. In some embodiments, provided herein are compositions and methods to modulate alternative splicing of SYNGAP1 pre-mRNA. In some embodiments, provided herein are compositions and methods to modulate alternative splicing in the splicing of SYNGAP1 pre-mRNA, e.g., to prevent inclusion of a NSAE during splicing of SYNGAP1 pre-mRNA and preserve canonical exons.

In various embodiments of the present disclosure, compositions and methods comprising a therapeutic agent are provided to modulate protein expression level of the target. In some embodiments, provided herein are compositions and methods to modulate alternative splicing of the target pre-mRNA. In some embodiments, provided herein are compositions and methods to modulate alternative splicing in the splicing of the target pre-mRNA, e.g., to prevent inclusion of a NSAE during splicing of the target pre-mRNA and preserve canonical exons, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14.

A therapeutic agent disclosed herein can be an alternative splicing repressor agent. In some embodiments, a therapeutic agent may comprise a polynucleic acid polymer. In other embodiments, a therapeutic agent may comprise a small molecule. In other embodiments, a therapeutic agent may comprise a polypeptide. In some embodiments, the therapeutic agent is a nucleic acid binding protein, with or without being complexed with a nucleic acid molecule. In other embodiments, the therapeutic agent is a nucleic acid molecule that encodes for another therapeutic agent. In further embodiments, the therapeutic agent is incorporated into a viral delivery system, such as an adenovirus-associated vector.

According to one aspect of the present disclosure, provided herein is a method of treatment or prevention of a condition associated with a functional-SYNGAP1 protein deficiency, comprising administering an alternative splicing repressor agent to a subject to increase levels of functional SYNGAP1 protein, wherein the agent binds to a region of the pre-mRNA transcript to decrease inclusion of the NSAE in the mature transcript. For example, provided herein is a method of treatment or prevention of a condition associated with a functional-SYNGAP1 protein deficiency, comprising administering an alternative splicing repressor agent to a subject to increase levels of functional SYNGAP1 protein, wherein the agent binds to a region of an exon or an intron (e.g., exon 10 or 11, intron 10 or intron 11 in human SYNGAP1 gene) of the pre-mRNA transcript.

According to one aspect of the present disclosure, provided herein is a method of treatment or prevention of a condition associated with a functional target protein deficiency, comprising administering an alternative splicing repressor agent to a subject to increase levels of functional target protein, wherein the agent binds to a region of the pre-mRNA transcript to decrease inclusion of the NSAE in the mature transcript. For example, provided herein is a method of treatment or prevention of a condition associated with a functional target protein deficiency, comprising administering an alternative splicing repressor agent to a subject to increase levels of functional target protein, wherein the agent binds to a region of an exon or an intron of the pre-mRNA transcript, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14.

In some embodiments, provided herein is a method of treatment or prevention of a condition associated with a functional-SYNGAP1 protein overexpression, comprising administering an alternative splicing modulator agent to a subject to decrease levels of functional SYNGAP1 protein, wherein the agent binds to a region of the pre-mRNA transcript to increase inclusion of the NSAE in the mature transcript. For example, provided herein is a method of treatment or prevention of a condition associated with a functional-SYNGAP1 protein overexpression, comprising administering an alternative splicing modulator agent to a subject to decrease levels of functional SYNGAP1 protein, wherein the agent binds to a region of an exon or an intron (e.g., exon 10 or 11, intron 10 or intron 11 in human SYNGAP1 gene) of the pre-mRNA transcript.

According to one aspect of the present disclosure, provided herein is a method of treatment or prevention of a condition associated with a functional target protein overexpression, comprising administering an alternative splicing modulator agent to a subject to decrease levels of functional target protein, wherein the agent binds to a region of the pre-mRNA transcript to increase inclusion of the NSAE in the mature transcript. For example, provided herein is a method of treatment or prevention of a condition associated with a functional target protein overexpression, comprising administering an alternative splicing repressor agent to a subject to decrease levels of functional target protein, wherein the agent binds to a region of an exon or an intron of the pre-mRNA transcript, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14.

Where reference is made to reducing alternative splicing in the mature mRNA, the reduction may be complete, e.g., 100%, or may be partial. The reduction may be clinically significant. The reduction/correction may be relative to the level of alternative splicing in the subject without treatment, or relative to the amount of alternative splicing in a population of similar subjects. The reduction/correction may be at least 10% less alternative splicing relative to the average subject, or the subject prior to treatment. The reduction may be at least 20% less alternative splicing relative to an average subject, or the subject prior to treatment. The reduction may be at least 40% less alternative splicing relative to an average subject, or the subject prior to treatment. The reduction may be at least 50% less alternative splicing relative to an average subject, or the subject prior to treatment. The reduction may be at least 60% less alternative splicing relative to an average subject, or the subject prior to treatment. The reduction may be at least 80% less alternative splicing relative to an average subject, or the subject prior to treatment. The reduction may be at least 90% less alternative splicing relative to an average subject, or the subject prior to treatment.

Where reference is made to increasing alternative splicing in the mature mRNA, the increase may be complete, e.g., 100%, or may be partial. The increase may be clinically significant. The increase/correction may be relative to the level of alternative splicing in the subject without treatment, or relative to the amount of alternative splicing in a population of similar subjects. The increase/correction may be at least 10% more alternative splicing relative to the average subject, or the subject prior to treatment. The increase may be at least 20% more alternative splicing relative to an average subject, or the subject prior to treatment. The increase may be at least 40% more alternative splicing relative to an average subject, or the subject prior to treatment. The increase may be at least 50% more alternative splicing relative to an average subject, or the subject prior to treatment. The increase may be at least 60% more alternative splicing relative to an average subject, or the subject prior to treatment. The increase may be at least 80% more alternative splicing relative to an average subject, or the subject prior to treatment. The increase may be at least 90% more alternative splicing relative to an average subject, or the subject prior to treatment.

Where reference is made to increasing functional-SYNGAP1 protein levels, the increase may be clinically significant. The increase may be relative to the level of functional-SYNGAP1 protein in the subject without treatment, or relative to the amount of functional-SYNGAP1 protein in a population of similar subjects. The increase may be at least 10% more functional-SYNGAP1 protein relative to the average subject, or the subject prior to treatment. The increase may be at least 20% more functional-SYNGAP1 protein relative to the average subject, or the subject prior to treatment. The increase may be at least 40% more functional-SYNGAP1 protein relative to the average subject, or the subject prior to treatment. The increase may be at least 50% more functional-SYNGAP1 protein relative to the average subject, or the subject prior to treatment. The increase may be at least 80% more functional-SYNGAP1 protein relative to the average subject, or the subject prior to treatment. The increase may be at least 100% more functional-SYNGAP1 protein relative to the average subject, or the subject prior to treatment. The increase may be at least 200% more functional-SYNGAP1 protein relative to the average subject, or the subject prior to treatment. The increase may be at least 500% more functional-SYNGAP1 protein relative to the average subject, or the subject prior to treatment.

Where reference is made to decreasing functional-SYNGAP1 protein levels, the decrease may be clinically significant. The decrease may be relative to the level of functional-SYNGAP1 protein in the subject without treatment, or relative to the amount of functional-SYNGAP1 protein in a population of similar subjects. The decrease may be at least 10% less functional-SYNGAP1 protein relative to the average subject, or the subject prior to treatment. The decrease may be at least 20% less functional-SYNGAP1 protein relative to the average subject, or the subject prior to treatment. The decrease may be at least 40% less functional-SYNGAP1 protein relative to the average subject, or the subject prior to treatment. The decrease may be at least 50% less functional-SYNGAP1 protein relative to the average subject, or the subject prior to treatment. The decrease may be at least 80% less functional-SYNGAP1 protein relative to the average subject, or the subject prior to treatment. The decrease may be at least 100% less functional-SYNGAP1 protein relative to the average subject, or the subject prior to treatment.

Where reference is made to increasing functional target protein levels, the increase may be clinically significant, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. The increase may be relative to the level of functional target protein in the subject without treatment, or relative to the amount of functional target protein in a population of similar subjects. The increase may be at least 10% more functional target protein relative to the average subject, or the subject prior to treatment. The increase may be at least 20% more functional target protein relative to the average subject, or the subject prior to treatment. The increase may be at least 40% more functional target protein relative to the average subject, or the subject prior to treatment. The increase may be at least 50% more functional target protein relative to the average subject, or the subject prior to treatment. The increase may be at least 80% more functional target protein relative to the average subject, or the subject prior to treatment. The increase may be at least 100% more functional target protein relative to the average subject, or the subject prior to treatment. The increase may be at least 200% more functional target protein relative to the average subject, or the subject prior to treatment. The increase may be at least 500% more functional target protein relative to the average subject, or the subject prior to treatment.

Where reference is made to decreasing functional target protein levels, the decrease may be clinically significant, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. The decrease may be relative to the level of functional target protein in the subject without treatment, or relative to the amount of functional target protein in a population of similar subjects. The decrease may be at least 10% less functional target protein relative to the average subject, or the subject prior to treatment. The decrease may be at least 20% less functional target protein relative to the average subject, or the subject prior to treatment. The decrease may be at least 40% less functional target protein relative to the average subject, or the subject prior to treatment. The decrease may be at least 50% less functional target protein relative to the average subject, or the subject prior to treatment. The decrease may be at least 80% less functional target protein relative to the average subject, or the subject prior to treatment. The decrease may be at least 100% less functional target protein relative to the average subject, or the subject prior to treatment.

In embodiments wherein the alternative splicing repressor agent comprises a polynucleic acid polymer, the polynucleic acid polymer may be about 50 nucleotides in length. In embodiments wherein the alternative splicing modulator agent comprises a polynucleic acid polymer, the polynucleic acid polymer may be about 50 nucleotides in length. The polynucleic acid polymer may be about 45 nucleotides in length. The polynucleic acid polymer may be about 40 nucleotides in length. The polynucleic acid polymer may be about 35 nucleotides in length. The polynucleic acid polymer may be about 30 nucleotides in length. The polynucleic acid polymer may be about 24 nucleotides in length. The polynucleic acid polymer may be about 25 nucleotides in length. The polynucleic acid polymer may be about 20 nucleotides in length. The polynucleic acid polymer may be about 19 nucleotides in length. The polynucleic acid polymer may be about 18 nucleotides in length. The polynucleic acid polymer may be about 17 nucleotides in length. The polynucleic acid polymer may be about 16 nucleotides in length. The polynucleic acid polymer may be about 15 nucleotides in length. The polynucleic acid polymer may be about 14 nucleotides in length. The polynucleic acid polymer may be about 13 nucleotides in length. The polynucleic acid polymer may be about 12 nucleotides in length. The polynucleic acid polymer may be about 11 nucleotides in length. The polynucleic acid polymer may be about 10 nucleotides in length. The polynucleic acid polymer may be between about 10 and about 50 nucleotides in length. The polynucleic acid polymer may be between about 10 and about 45 nucleotides in length. The polynucleic acid polymer may be between about 10 and about 40 nucleotides in length. The polynucleic acid polymer may be between about 10 and about 35 nucleotides in length. The polynucleic acid polymer may be between about 10 and about 30 nucleotides in length. The polynucleic acid polymer may be between about 10 and about 25 nucleotides in length. The polynucleic acid polymer may be between about 10 and about 20 nucleotides in length. The polynucleic acid polymer may be between about 15 and about 25 nucleotides in length. The polynucleic acid polymer may be between about 15 and about 30 nucleotides in length. The polynucleic acid polymer may be between about 12 and about 30 nucleotides in length.

The sequence of the polynucleic acid polymer may be at least 50%, 55%, 60%, 65%, 70%, 75%, 80%, 85%, 90%, 91%, 92%, 93%, 94%, 95%, 96%, 97%, 98%, 99%, or 99.5% complementary to a target sequence of an mRNA transcript, e.g., a partially processed mRNA transcript. The sequence of the polynucleic acid polymer may be 100% complementary to a target sequence of a pre-mRNA transcript.

The sequence of the polynucleic acid polymer may have 4 or fewer mismatches to a target sequence of the pre-mRNA transcript. The sequence of the polynucleic acid polymer may have 3 or fewer mismatches to a target sequence of the pre-mRNA transcript. The sequence of the polynucleic acid polymer may have 2 or fewer mismatches to a target sequence of the pre-mRNA transcript. The sequence of the polynucleic acid polymer may have 1 or fewer mismatches to a target sequence of the pre-mRNA transcript. The sequence of the polynucleic acid polymer may have no mismatches to a target sequence of the pre-mRNA transcript.

In some embodiments, the polynucleic acid polymer may specifically hybridize to a target sequence of the pre-mRNA transcript. For example, the polynucleic acid polymer may have 91%, 92%, 93%, 94%, 95%, 96%, 97%, 98%, 99%, 99.5% or 100% sequence complementarity to a target sequence of the pre-mRNA transcript. The hybridization may be under high stringent hybridization conditions.

The polynucleic acid polymer may have a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to a sequence of Table 1, Table 6A, and Table 6B. The polynucleic acid polymer may have a sequence with 100% sequence identity to a sequence selected from the group consisting of Table 1, Table 6A, and Table 6B. In some embodiments, the polynucleic acid polymer comprises a sequence with at least 50%, 55%, 60%, 65%, 70%, 75%, 80%, 85%, 90%, 91%, 92%, 93%, 94%, 95%, 96%, 97%, 98%, 99%, or 99.5% sequence identity to any one selected from the group consisting of SEQ ID NO: 1-SEQ ID NO: 165, and SEQ ID NO: 1301-SEQ ID NO: 10225. In some embodiments, the polynucleic acid polymer comprises a sequence with 100% sequence identity to any one selected from the group consisting of SEQ ID NO: 1-SEQ ID NO: 165, and SEQ ID NO: 1301-SEQ ID NO: 10225. In some embodiments the polynucleic acid polymer is a polynucleic acid polymer of at least 50%, 55%, 60%, 65%, 70%, 75%, 80%, 85%, 90%, 91%, 92%, 93%, 94%, 95%, 96%, 97%, 98%, 99%, or 99.5% sequence identity to any one selected from the group consisting of SEQ ID NO: 1-SEQ ID NO: 165, and SEQ ID NO: 1301-SEQ ID NO: 10225. In some embodiments, the polynucleic acid polymer is a polynucleic acid polymer of any one selected from the group consisting of SEQ ID NO: 1-SEQ ID NO: 165, and SEQ ID NO: 1301-SEQ ID NO: 10225.

TABLE 1
(gene = SYNGAP1)

| ASO (Bolde = 2′MOE, | | | | | | | |
|---|---|---|---|---|---|---|---|
| Italicized = LNA, | SEQ | ASO sequence with | SEQ | | | | Name |
| underlined C = 5methylC) | ID | no mismatches | ID | Coordinate | | | Position |
| Sequence 5′-3′ | NO: | Sequence 5′-3′ | NO: | Chr | Start | End | ID |
| ATGGGAGAGAGATGGAGG | 1 | | | chr6 | 33440369 | 33440387 | SYNGAP |
| | | | | | | | 1-IVS10- |
| | | | | | | | 166 |
| CAGAGATGGGAGAGAGAT | 2 | | | chr6 | 33440374 | 33440392 | SYNGAP |
| | | | | | | | 1-IVS10- |
| | | | | | | | 161 |
| AGAGACAGAGATGGGAGA | 3 | | | chr6 | 33440379 | 33440397 | SYNGAP |
| | | | | | | | 1-IVS10- |
| | | | | | | | 156 |
| CAGGGAGAGACAGAGATG | 4 | | | chr6 | 33440384 | 33440402 | SYNGAP |
| | | | | | | | 1-IVS10- |
| | | | | | | | 151 |
| GACACCAGGGAGAGACAG | 5 | | | chr6 | 33440389 | 33440407 | SYNGAP |
| | | | | | | | 1-IVS10- |
| | | | | | | | 146 |
| AAACAGACACCAGGGAGA | 6 | | | chr6 | 33440394 | 33440412 | SYNGAP |
| | | | | | | | 1-IVS10- |
| | | | | | | | 141 |
| AAGAAAAACAGACACCAG | 7 | | | chr6 | 33440399 | 33440417 | SYNGAP |
| | | | | | | | 1-IVS10- |
| | | | | | | | 136 |
| AGGAGAAGAAAAACAGAC | 8 | | | chr6 | 33440404 | 33440422 | SYNGAP |
| | | | | | | | 1-IVS10- |
| | | | | | | | 131 |
| AGAGGAGGAGAAGAAAAA | 9 | | | chr6 | 33440409 | 33440427 | SYNGAP |
| | | | | | | | 1-IVS10- |
| | | | | | | | 126 |
| CAAGGAGAGGAGGAGAAG | 10 | | | chr6 | 33440414 | 33440432 | SYNGAP |
| | | | | | | | 1-IVS10- |
| | | | | | | | 121 |
| AGAGACAAGGAGAGGAGG | 11 | | | chr6 | 33440419 | 33440437 | SYNGAP |
| | | | | | | | 1-IVS10- |
| | | | | | | | 116 |
| GGGAGAGAGACAAGGAGA | 12 | | | chr6 | 33440424 | 33440442 | SYNGAP |
| | | | | | | | 1-IVS10- |
| | | | | | | | 111 |
| GGTGTGGGAGAGAGACAA | 13 | | | chr6 | 33440429 | 33440447 | SYNGAP |
| | | | | | | | 1-IVS10- |
| | | | | | | | 106 |
| GGAGGGTTGTGGGAGAGA | 14 | GGAGGGGTGTG | 144 | chr6 | 33440434 | 33440452 | SYNGAP |
| | | GGAGAGA | | | | | 1-IVS10- |
| | | | | | | | 101 |
| GAGATGGATGGGTGTGGG | 15 | GAGATGGAGGG | 145 | chr6 | 33440439 | 33440457 | SYNGAP |
| | | GTGTGGG | | | | | 1-IVS10- |
| | | | | | | | 96 |
| GGAGAGAGATGGATGGGT | 16 | GGAGAGAGATG | 146 | chr6 | 33440444 | 33440462 | SYNGAP |
| | | GAGGGGT | | | | | 1-IVS10- |
| | | | | | | | 91 |
| ACGTGGGAGAGAGATGGA | 17 | | | chr6 | 33440449 | 33440467 | SYNGAP |
| | | | | | | | 1-IVS10- |
| | | | | | | | 86 |
| GAGACACGTGGGAGAGAG | 18 | | | chr6 | 33440454 | 33440472 | SYNGAP |
| | | | | | | | 1-IVS10- |
| | | | | | | | 81 |
| AGGGAGAGACACGTGGGA | 19 | GGGGAGAGACA | 147 | chr6 | 33440459 | 33440477 | SYNGAP |
| | | CGTGGGA | | | | | 1-IVS10- |
| | | | | | | | 76 |
| GGTGAGGGAAGAGACACG | 20 | GGTGAGGGGAG | 148 | chr6 | 33440464 | 33440482 | SYNGAP |
| | | AGACACG | | | | | 1-IVS10- |
| | | | | | | | 71 |
| GAGAAGGTGAAGGGAGAG | 21 | GAGAAGGTGAG | 149 | chr6 | 33440469 | 33440487 | SYNGAP |
| | | GGGAGAG | | | | | 1-IVS10- |
| | | | | | | | 66 |
| AGGGAGAGAAGGTGAGGG | 22 | GGGGAGAGAAG | 150 | chr6 | 33440474 | 33440492 | SYNGAP |
| | | GTGAGGG | | | | | 1-IVS10- |
| | | | | | | | 61 |
| TGGAGGGAGAGAGAAGGT | 23 | TGGAGGGGGAG | 151 | chr6 | 33440479 | 33440497 | SYNGAP |
| | | AGAAGGT | | | | | 1-IVS10- |
| | | | | | | | 56 |
| AGAAATGGAGAGGGAGAG | 24 | AGAAATGGAGG | 152 | chr6 | 33440484 | 33440502 | SYNGAP |
| | | GGGAGAG | | | | | 1-IVS10- |
| | | | | | | | 51 |
| GAGAGAGAAATGGAGGGA | 25 | GAGAGAGAAAT | 153 | chr6 | 33440489 | 33440507 | SYNGAP |
| | | GGAGGGG | | | | | 1-IVS10- |
| | | | | | | | 46 |
| TTAGGGAGAGAGAAATGG | 26 | | | chr6 | 33440494 | 33440512 | SYNGAP |
| | | | | | | | 1-IVS10- |
| | | | | | | | 41 |
| ACAGATTAGGGAGAGAGA | 27 | | | chr6 | 33440499 | 33440517 | SYNGAP |
| | | | | | | | 1-IVS10- |
| | | | | | | | 36 |
| AACAGACAGATTAGGGAG | 28 | | | chr6 | 33440504 | 33440522 | SYNGAP |
| | | | | | | | 1-IVS10- |
| | | | | | | | 31 |
| GAGGGAACAGACAGATTA | 29 | | | chr6 | 33440509 | 33440527 | SYNGAP |
| | | | | | | | 1-IVS10- |
| | | | | | | | 26 |
| TGGCAGAGGGAACAGACA | 30 | | | chr6 | 33440514 | 33440532 | SYNGAP |
| | | | | | | | 1-IVS10- |
| | | | | | | | 21 |
| GGCCATGGCAGAGGGAAC | 31 | | | chr6 | 33440519 | 33440537 | SYNGAP |
| | | | | | | | 1-IVS10- |
| | | | | | | | 16 |
| AAGGGAGCCATGGCAGAG | 32 | AAGGGGGCCAT | 154 | chr6 | 33440524 | 33440542 | SYNGAP |
| | | GGCAGAG | | | | | 1-IVS10- |
| | | | | | | | 11 |
| TGAAGAAGGAGGCCATGG | 33 | TGAAGAAGGGG | 155 | chr6 | 33440529 | 33440547 | SYNGAP |
| | | GCCATGG | | | | | 1-IVS10-6 |
| CTGCTTGAAGAAGAGGGC | 34 | CTGCTTGAAGA | 156 | chr6 | 33440534 | 33440552 | SYNGAP |
| | | AGGGGGC | | | | | 1-IVS10-1 |
| GGAGGCTGCTTGAAGAAG | 35 | | | chr6 | 33440539 | 33440557 | SYNGAP |
| | | | | | | | 1- |
| | | | | | | | I10AltE11 |
| | | | | | | | +5 |
| AGATGGGAGGCTGCTTGA | 36 | | | chr6 | 33440544 | 33440562 | SYNGAP |
| | | | | | | | 1- |
| | | | | | | | I10AltE11 |
| | | | | | | | +10 |
| GAGCAAGATGGGAGGCTG | 37 | | | chr6 | 33440549 | 33440567 | SYNGAP |
| | | | | | | | 1- |
| | | | | | | | I10AltE11 |
| | | | | | | | +15 |
| CAGGAGCAAGATGGGAGG | 38 | | | chr6 | 33440552 | 33440570 | SYNGAP |
| | | | | | | | 1- |
| | | | | | | | AltEx11+ |
| | | | | | | | 1 |
| GACCGCAGGAGCAAGATG | 39 | | | chr6 | 33440557 | 33440575 | SYNGAP |
| | | | | | | | 1- |
| | | | | | | | AltEx11+ |
| | | | | | | | 6 |
| GGAGGGACCGCAGGAGCA | 40 | | | chr6 | 33440562 | 33440580 | SYNGAP |
| | | | | | | | 1- |
| | | | | | | | AltEx11+ |
| | | | | | | | 11 |
| GGGAAGGAGGGACCGCAG | 41 | | | chr6 | 33440567 | 33440585 | SYNGAP |
| | | | | | | | 1- |
| | | | | | | | AltEx11+ |
| | | | | | | | 16 |
| AGACAGGGAAGGAGGGAC | 42 | | | chr6 | 33440572 | 33440590 | SYNGAP |
| | | | | | | | 1- |
| | | | | | | | AltEx11+ |
| | | | | | | | 21 |
| GAGAGAGACAGGGAAGGA | 43 | | | chr6 | 33440577 | 33440595 | SYNGAP |
| | | | | | | | 1- |
| | | | | | | | AltEx11+ |
| | | | | | | | 26 |
| AGGGTGAGAGAGACAGGG | 44 | GGGGTGAGAGA | 157 | chr6 | 33440582 | 33440600 | SYNGAP |
| | | GACAGGG | | | | | 1- |
| | | | | | | | AltEx11+ |
| | | | | | | | 31 |
| AAACAGGGATGAGAGAGA | 45 | AAACAGGGGTG | 158 | chr6 | 33440587 | 33440605 | SYNGAP |
| | | AGAGAGA | | | | | 1- |
| | | | | | | | AltEx11+ |
| | | | | | | | 36 |
| TGTGGAAACAAGGGTGAG | 46 | TGTGGAAACAG | 159 | chr6 | 33440592 | 33440610 | SYNGAP |
| | | GGGTGAG | | | | | 1- |
| | | | | | | | AltEx11+ |
| | | | | | | | 41 |
| GAGGGTGTGGAAACAGGG | 47 | | | chr6 | 33440597 | 33440615 | SYNGAP |
| | | | | | | | 1- |
| | | | | | | | AltEx11+ |
| | | | | | | | 46 |
| GAGGTGAGGGTGTGGAAA | 48 | | | chr6 | 33440602 | 33440620 | SYNGAP |
| | | | | | | | 1- |
| | | | | | | | AltEx11+ |
| | | | | | | | 51 |
| GGTAGGAGGTGAGGGTGT | 49 | | | chr6 | 33440607 | 33440625 | SYNGAP |
| | | | | | | | 1- |
| | | | | | | | AltEx11+ |
| | | | | | | | 56 |
| AGGGTGGTAGGAGGTGAG | 50 | GGGGTGGTAGG | 160 | chr6 | 33440612 | 33440630 | SYNGAP |
| | | AGGTGAG | | | | | 1- |
| | | | | | | | AltEx11+ |
| | | | | | | | 61 |
| TGAGGGAGGTGGTAGGAG | 51 | TGAGGGGGGTG | 161 | chr6 | 33440617 | 33440635 | SYNGAP |
| | | GTAGGAG | | | | | 1- |
| | | | | | | | AltEx11+ |
| | | | | | | | 66 |
| CATGCTGAGGAGGGTGGT | 52 | CATGCTGAGGG | 162 | chr6 | 33440622 | 33440640 | SYNGAP |
| | | GGGTGGT | | | | | 1- |
| | | | | | | | AltEx11+ |
| | | | | | | | 71 |
| GGGAACATGCTGAGAGGG | 53 | GGGAACATGCT | 163 | chr6 | 33440627 | 33440645 | SYNGAP |
| | | GAGGGGG | | | | | 1- |
| | | | | | | | AltEx11+ |
| | | | | | | | 76 |
| TTCCAGGGAACATGCTGA | 54 | | | chr6 | 33440632 | 33440650 | SYNGAP |
| | | | | | | | 1- |
| | | | | | | | AltEx11+ |
| | | | | | | | 81 |
| TCAGCTTCCAGGGAACAT | 55 | | | chr6 | 33440637 | 33440655 | SYNGAP |
| | | | | | | | 1- |
| | | | | | | | AltEx11+ |
| | | | | | | | 86 |
| GGGAGAGAGATGGATGGG | 56 | GGGAGAGAGAT | 164 | chr6 | 33440445 | 33440463 | SYNGAP |
| | | GGAGGGG | | | | | 1-IVS10- |
| | | | | | | | 90 |
| TGGGAGAGAGATGGAGGG | 57 | | | chr6 | 33440446 | 33440464 | SYNGAP |
| | | | | | | | 1-IVS10- |
| | | | | | | | 89 |
| GTGGGAGAGAGATGGAGG | 58 | | | chr6 | 33440447 | 33440465 | SYNGAP |
| | | | | | | | 1-IVS10- |
| | | | | | | | 88 |
| CGTGGGAGAGAGATGGAG | 59 | | | chr6 | 33440448 | 33440466 | SYNGAP |
| | | | | | | | 1-IVS10- |
| | | | | | | | 87 |
| CACGTGGGAGAGAGATGG | 60 | | | chr6 | 33440450 | 33440468 | SYNGAP |
| | | | | | | | 1-IVS10- |
| | | | | | | | 85 |
| ACACGTGGGAGAGAGATG | 61 | | | chr6 | 33440451 | 33440469 | SYNGAP |
| | | | | | | | 1-IVS10- |
| | | | | | | | 84 |
| GACACGTGGGAGAGAGAT | 62 | | | chr6 | 33440452 | 33440470 | SYNGAP |
| | | | | | | | 1-IVS10- |
| | | | | | | | 83 |
| AGACACGTGGGAGAGAGA | 63 | | | chr6 | 33440453 | 33440471 | SYNGAP |
| | | | | | | | 1-IVS10- |
| | | | | | | | 82 |
| AGAGACACGTGGGAGAGA | 64 | | | chr6 | 33440455 | 33440473 | SYNGAP |
| | | | | | | | 1-IVS10- |
| | | | | | | | 80 |
| GAGAGACACGTGGGAGAG | 65 | | | chr6 | 33440456 | 33440474 | SYNGAP |
| | | | | | | | 1-IVS10- |
| | | | | | | | 79 |
| GGAGAGACACGTGGGAGA | 66 | | | chr6 | 33440457 | 33440475 | SYNGAP |
| | | | | | | | 1-IVS10- |
| | | | | | | | 78 |
| GGGAGAGACACGTGGGAG | 67 | | | chr6 | 33440458 | 33440476 | SYNGAP |
| | | | | | | | 1-IVS10- |
| | | | | | | | 77 |
| AGGGAACATGCTGATGGG | 68 | AGGGAACATGC | 165 | chr6 | 33440628 | 33440646 | SYNGAP |
| | | TGAGGGG | | | | | 1- |
| | | | | | | | AltEX11+ |
| | | | | | | | 77 |
| CAGGGAACATGCTGAGGG | 69 | | | chr6 | 33440629 | 33440647 | SYNGAP |
| | | | | | | | 1- |
| | | | | | | | AltEX11+ |
| | | | | | | | 78 |
| CCAGGGAACATGCTGAGG | 70 | | | chr6 | 33440630 | 33440648 | SYNGAP |
| | | | | | | | 1- |
| | | | | | | | AltEX11+ |
| | | | | | | | 79 |
| TCCAGGGAACATGCTGAG | 71 | | | chr6 | 33440631 | 33440649 | SYNGAP |
| | | | | | | | 1- |
| | | | | | | | AltEX11+ |
| | | | | | | | 80 |
| CTTCCAGGGAACATGCTG | 72 | | | chr6 | 33440633 | 33440651 | SYNGAP |
| | | | | | | | 1- |
| | | | | | | | AltEX11+ |
| | | | | | | | 82 |
| GCTTCCAGGGAACATGCT | 73 | | | chr6 | 33440634 | 33440652 | SYNGAP |
| | | | | | | | 1- |
| | | | | | | | AltEX11+ |
| | | | | | | | 83 |
| AGCTTCCAGGGAACATGC | 74 | | | chr6 | 33440635 | 33440653 | SYNGAP |
| | | | | | | | 1- |
| | | | | | | | AltEX11+ |
| | | | | | | | 84 |
| CAGCTTCCAGGGAACATG | 75 | | | chr6 | 33440636 | 33440654 | SYNGAP |
| | | | | | | | 1- |
| | | | | | | | AltEX11+ |
| | | | | | | | 85 |
| GAGACACGTGGGAGAG | 76 | | | chr6 | 33440456 | 33440472 | SYNGAP |
| | | | | | | | 1-IVS10- |
| | | | | | | | 81(16) |
| GACACGTGGGAGAGAG | 77 | | | chr6 | 33440454 | 33440470 | SYNGAP |
| | | | | | | | 1-IVS10- |
| | | | | | | | 83(16) |
| CACGTGGGAGAGAGAT | 78 | | | chr6 | 33440452 | 33440468 | SYNGAP |
| | | | | | | | 1-IVS10- |
| | | | | | | | 85(16) |
| CGTGGGAGAGAGATGG | 79 | | | chr6 | 33440450 | 33440466 | SYNGAP |
| | | | | | | | 1-IVS10- |
| | | | | | | | 87(16) |
| GTGGGAGAGAGATGGA | 80 | | | chr6 | 33440449 | 33440465 | SYNGAP |
| | | | | | | | 1-IVS10- |
| | | | | | | | 88(16) |
| GAGACACGTGGGAG | 81 | | | chr6 | 33440458 | 33440472 | SYNGAP |
| | | | | | | | 1-IVS10- |
| | | | | | | | 81(14) |
| GACACGTGGGAGAG | 82 | | | chr6 | 33440456 | 33440470 | SYNGAP |
| | | | | | | | 1-IVS10- |
| | | | | | | | 83(14) |
| CACGTGGGAGAGAG | 83 | | | chr6 | 33440454 | 33440468 | SYNGAP |
| | | | | | | | 1-IVS10- |
| | | | | | | | 85(14) |
| CGTGGGAGAGAGAT | 84 | | | chr6 | 33440452 | 33440466 | SYNGAP |
| | | | | | | | 1-IVS10- |
| | | | | | | | 87(14) |
| TGGGAGAGAGATGG | 85 | | | chr6 | 33440450 | 33440464 | SYNGAP |
| | | | | | | | 1-IVS10- |
| | | | | | | | 89(14) |
| GGGAGAGAGATGGA | 86 | | | chr6 | 33440449 | 33440463 | SYNGAP |
| | | | | | | | 1-IVS10- |
| | | | | | | | 90(14) |
| GAGACACGTGGG | 87 | | | chr6 | 33440460 | 33440472 | SYNGAP |
| | | | | | | | 1-IVS10- |
| | | | | | | | 81(12) |
| GACACGTGGGAG | 88 | | | chr6 | 33440458 | 33440470 | SYNGAP |
| | | | | | | | 1-IVS10- |
| | | | | | | | 83(12) |
| CACGTGGGAGAG | 89 | | | chr6 | 33440456 | 33440468 | SYNGAP |
| | | | | | | | 1-IVS10- |
| | | | | | | | 85(12) |
| CGTGGGAGAGAG | 90 | | | chr6 | 33440454 | 33440466 | SYNGAP |
| | | | | | | | 1-IVS10- |
| | | | | | | | 87(12) |
| TGGGAGAGAGAT | 91 | | | chr6 | 33440452 | 33440464 | SYNGAP |
| | | | | | | | 1-IVS10- |
| | | | | | | | 89(12) |
| GGAGAGAGATGG | 92 | | | chr6 | 33440450 | 33440462 | SYNGAP |
| | | | | | | | 1-IVS10- |
| | | | | | | | 91(12) |
| GAGAGAGATGGA | 93 | | | chr6 | 33440449 | 33440461 | SYNGAP |
| | | | | | | | 1-IVS10- |
| | | | | | | | 92(12) |
| CCAGGGAGAGACAGAGAT | 94 | | | chr6 | 33440385 | 33440403 | SYNGAP |
| | | | | | | | 1-IVS10- |
| | | | | | | | 150 |
| ACCAGGGAGAGACAGAGA | 95 | | | chr6 | 33440386 | 33440404 | SYNGAP |
| | | | | | | | 1-IVS10- |
| | | | | | | | 149 |
| CACCAGGGAGAGACAGAG | 96 | | | chr6 | 33440387 | 33440405 | SYNGAP |
| | | | | | | | 1-IVS10- |
| | | | | | | | 148 |
| ACACCAGGGAGAGACAGA | 97 | | | chr6 | 33440388 | 33440406 | SYNGAP |
| | | | | | | | 1-IVS10- |
| | | | | | | | 147 |
| AGACACCAGGGAGAGACA | 98 | | | chr6 | 33440390 | 33440408 | SYNGAP |
| | | | | | | | 1-IVS10- |
| | | | | | | | 145 |
| CAGACACCAGGGAGAGAC | 99 | | | chr6 | 33440391 | 33440409 | SYNGAP |
| | | | | | | | 1-IVS10- |
| | | | | | | | 144 |
| ACAGACACCAGGGAGAGA | 100 | | | chr6 | 33440392 | 33440410 | SYNGAP |
| | | | | | | | 1-IVS10- |
| | | | | | | | 143 |
| AACAGACACCAGGGAGAG | 101 | | | chr6 | 33440393 | 33440411 | SYNGAP |
| | | | | | | | 1-IVS10- |
| | | | | | | | 142 |
| AAAACAGACACCAGGGAG | 102 | | | chr6 | 33440395 | 33440413 | SYNGAP |
| | | | | | | | 1-IVS10- |
| | | | | | | | 140 |
| AAAAACAGACACCAGGGA | 103 | | | chr6 | 33440396 | 33440414 | SYNGAP |
| | | | | | | | 1-IVS10- |
| | | | | | | | 139 |
| GAAAAACAGACACCAGGG | 104 | | | chr6 | 33440397 | 33440415 | SYNGAP |
| | | | | | | | 1-IVS10- |
| | | | | | | | 138 |
| AGAAAAACAGACACCAGG | 105 | | | chr6 | 33440398 | 33440416 | SYNGAP |
| | | | | | | | 1-IVS10- |
| | | | | | | | 137 |
| GAGACAGGGAAGGAGGGA | 106 | | | chr6 | 33440573 | 33440591 | SYNGAP |
| | | | | | | | 1- |
| | | | | | | | AltEx11+ |
| | | | | | | | 22 |
| AGAGACAGGGAAGGAGGG | 107 | | | chr6 | 33440574 | 33440592 | SYNGAP |
| | | | | | | | 1- |
| | | | | | | | AltEx11+ |
| | | | | | | | 23 |
| GAGAGACAGGGAAGGAGG | 108 | | | chr6 | 33440575 | 33440593 | SYNGAP |
| | | | | | | | 1- |
| | | | | | | | AltEx11+ |
| | | | | | | | 24 |
| AGAGAGACAGGGAAGGAG | 109 | | | chr6 | 33440576 | 33440594 | SYNGAP |
| | | | | | | | 1- |
| | | | | | | | AltEx11+ |
| | | | | | | | 25 |
| TGAGAGAGACAGGGAAGG | 110 | | | chr6 | 33440578 | 33440596 | SYNGAP |
| | | | | | | | 1- |
| | | | | | | | AltEx11+ |
| | | | | | | | 27 |
| GTGAGAGAGACAGGGAAG | 111 | | | chr6 | 33440579 | 33440597 | SYNGAP |
| | | | | | | | 1- |
| | | | | | | | AltEx11+ |
| | | | | | | | 28 |
| GGTGAGAGAGACAGGGAA | 112 | | | chr6 | 33440580 | 33440598 | SYNGAP |
| | | | | | | | 1- |
| | | | | | | | AltEx11+ |
| | | | | | | | 29 |
| GGGTGAGAGAGACAGGGA | 113 | | | chr6 | 33440581 | 93344059 | SYNGAP |
| | | | | | | | 1- |
| | | | | | | | AltEx11+ |
| | | | | | | | 30 |
| TTCCAGGGAACATGCTGA | 114 | | | chr6 | 33440630 | 33440650 | SYNGAP |
| GG | | | | | | | 1- |
| | | | | | | | AltEX11+ |
| | | | | | | | 79 |
| AGGGAACATGCTGAGG | 115 | | | chr6 | 33440630 | 33440646 | SYNGAP |
| | | | | | | | 1- |
| | | | | | | | AltEX11+ |
| | | | | | | | 79 |
| CAGGGAACATGCTGAG | 116 | | | chr6 | 33440631 | 33440647 | SYNGAP |
| | | | | | | | 1- |
| | | | | | | | AltEX11+ |
| | | | | | | | 80 |
| CCAGGGAACATGCTGA | 117 | | | chr6 | 33440632 | 33440648 | SYNGAP |
| | | | | | | | 1- |
| | | | | | | | AltEX11+ |
| | | | | | | | 81 |
| TCCAGGGAACATGCTG | 118 | | | chr6 | 33440633 | 33440649 | SYNGAP |
| | | | | | | | 1- |
| | | | | | | | AltEX11+ |
| | | | | | | | 82 |
| TTCCAGGGAACATGCT | 119 | | | chr6 | 33440634 | 33440650 | SYNGAP |
| | | | | | | | 1- |
| | | | | | | | AltEx11+ |
| | | | | | | | 83 |
| [symbol] A [symbol] GG [symbol] AC [symbol] TG [symbol] TG [symbol] GG | 120 | | | chr6 | 33440630 | 33440648 | SYNGAP |
| | | | | | | | 1- |
| | | | | | | | AltEX11+ |
| | | | | | | | 79 |
| [symbol] CC [symbol] GG [symbol] AA [symbol] AT [symbol] CT [symbol] AG | 121 | | | chr6 | 33440631 | 33440649 | SYNGAP |
| | | | | | | | 1- |
| | | | | | | | AltEX11+ |
| | | | | | | | 80 |
| [symbol] T [symbol] AG [symbol] GA [symbol] CA [symbol] GC [symbol] GA | 122 | | | chr6 | 33440632 | 33440650 | SYNGAP |
| | | | | | | | 1- |
| | | | | | | | AltEx11+ |
| | | | | | | | 81 |
| [symbol] GG [symbol] AA [symbol] AT [symbol] CT [symbol] AG [symbol] | 123 | | | chr6 | 33440630 | 33440646 | SYNGAP |
| | | | | | | | 1- |
| | | | | | | | AltEX11+ |
| | | | | | | | 79 |
| [symbol] AG [symbol] GA [symbol] CA [symbol] GC [symbol] GA [symbol] | 124 | | | chr6 | 33440631 | 33440647 | SYNGAP |
| | | | | | | | 1- |
| | | | | | | | AltEX11+ |
| | | | | | | | 80 |
| [symbol] A [symbol] GG [symbol] AC [symbol] TG [symbol] TG [symbol] | 125 | | | chr6 | 33440632 | 33440648 | SYNGAP |
| | | | | | | | 1- |
| | | | | | | | AltEX11+ |
| | | | | | | | 81 |
| [symbol] CC [symbol] GG [symbol] AA [symbol] AT [symbol] CT [symbol] | 126 | | | chr6 | 33440633 | 33440649 | SYNGAP |
| | | | | | | | 1- |
| | | | | | | | AltEX11+ |
| | | | | | | | 82 |
| [symbol] T [symbol] AG [symbol] GA [symbol] CA [symbol] GC [symbol] | 127 | | | chr6 | 33440634 | 33440650 | SYNGAP |
| | | | | | | | 1- |
| | | | | | | | AltEx11+ |
| | | | | | | | 83 |
| [symbol] GGG [symbol] AC [symbol] TG [symbol] TG [symbol] GG | 128 | | | chr6 | 33440630 | 33440648 | SYNGAP |
| | | | | | | | 1- |
| | | | | | | | AltEX11+ |
| | | | | | | | 79a |
| [symbol] CC [symbol] GG [symbol] AAC [symbol] T [symbol] CT [symbol] AG | 129 | | | chr6 | 33440631 | 33440649 | SYNGAP |
| | | | | | | | 1- |
| | | | | | | | AltEX11+ |
| | | | | | | | 80a |
| [symbol] TCC [symbol] GG [symbol] AAC [symbol] T [symbol] CTG [symbol] | 130 | | | chr6 | 33440632 | 33440650 | SYNGAP |
| | | | | | | | 1- |
| | | | | | | | AltEx11+ |
| | | | | | | | 81a |
| [symbol] GG [symbol] A [symbol] CAT [symbol] CT [symbol] AG [symbol] | 131 | | | chr6 | 33440630 | 33440646 | SYNGAP |
| | | | | | | | 1- |
| | | | | | | | AltEX11+ |
| | | | | | | | 79a |
| C [symbol] G [symbol] GA [symbol] CAT [symbol] CT [symbol] A [symbol] | 132 | | | chr6 | 33440631 | 33440647 | SYNGAP |
| | | | | | | | 1- |
| | | | | | | | AltEX11+ |
| | | | | | | | 80a |
| [symbol] GGG [symbol] AC [symbol] TG [symbol] TG [symbol] | 133 | | | chr6 | 33440632 | 33440648 | SYNGAP |
| | | | | | | | 1- |
| | | | | | | | AltEX11+ |
| | | | | | | | 81a |
| [symbol] CC [symbol] GG [symbol] A [symbol] CAT [symbol] CT [symbol] | 134 | | | chr6 | 33440633 | 33440649 | SYNGAP |
| | | | | | | | 1- |
| | | | | | | | AltEX11+ |
| | | | | | | | 82a |
| [symbol] TCC [symbol] G [symbol] GA [symbol] CAT [symbol] C [symbol] | 135 | | | chr6 | 33440634 | 33440650 | SYNGAP |
| | | | | | | | 1- |
| | | | | | | | AltEx11+ |
| | | | | | | | 83a |
| CAGACACCAGGGAGAGAC | 136 | | | chr6 | 33440389 | 33440409 | SYNGAP |
| AG | | | | | | | 1-IVS10- |
| | | | | | | | 146(20) |
| ACAGACACCAGGGAGAGA | 137 | | | chr6 | 33440389 | 33440410 | SYNGAP |
| CAG | | | | | | | 1-IVS10- |
| | | | | | | | 146(21) |
| AACAGACACCAGGGAGAG | 138 | | | chr6 | 33440389 | 33440411 | SYNGAP |
| ACAG | | | | | | | 1-IVS10- |
| | | | | | | | 146(22) |
| AAACAGACACCAGGGAGA | 139 | | | chr6 | 33440389 | 33440412 | SYNGAP |
| GACAG | | | | | | | 1-IVS10- |
| | | | | | | | 146(23) |
| CAGACACCAGGGAGAGAC | 140 | | | chr6 | 33440389 | 33440409 | SYNGAP |
| AG | | | | | | | 1-IVS10- |
| | | | | | | | 146(20)-P |
| ACAGACACCAGGGAGAGA | 141 | | | chr6 | 33440389 | 33440410 | SYNGAP |
| CAG | | | | | | | 1-IVS10- |
| | | | | | | | 146(21)-P |
| AACAGACACCAGGGAGAG | 142 | | | chr6 | 33440389 | 33440411 | SYNGAP |
| ACAG | | | | | | | 1-IVS10- |
| | | | | | | | 146(22)-P |
| AAACAGACACCAGGGAGA | 143 | | | chr6 | 33440389 | 33440412 | SYNGAP |
| GACAG | | | | | | | 1-IVS10- |
| | | | | | | | 146(23)-P |

TABLE 2
SYNGAP1

| | |
|---|---|
| Canonical intron | SEQ ID NO: 166 |
| chr6: 33438919-33440728 | |
| Alternative intron | SEQ ID NO: 167 |
| chr6: 33438919-33440552 | |
| Canonical exon | SEQ ID NO: 168 |
| chr6: 33440729-33440965 | |
| Alternative exon | SEQ ID NO: 169 |
| chr6: 33440553-33440965 | |

TABLE 3
ABCA5

| | |
|---|---|
| Canonical exon chr17: 50659237-50659368 | SEQ ID NO: 170 |
| Alternative exon chr17: 50659182-50659368 | SEQ ID NO: 171 |
| Canonical intron chr17: 50658497-50659236 | SEQ ID NO: 172 |
| Alternative intron chr17: 50658497-50659181 | SEQ ID NO: 173 |

ABCA7

| | |
|---|---|
| Canonical exon chr19: 1063760-1063863 | SEQ ID NO: 174 |
| Alternative exon chr19: 1063760-1063899 | SEQ ID NO: 175 |
| Canonical intron chr19: 1063864-1064160 | SEQ ID NO: 176 |
| Alternative intron chr19: 1063900-1064160 | SEQ ID NO: 177 |

ABCC3

| | |
|---|---|
| Canonical exon chr17: 50659237-50659368 | SEQ ID NO: 178 |
| Alternative exon chr17: 50659182-50659368 | SEQ ID NO: 179 |
| Canonical intron chr17: 50658497-50659236 | SEQ ID NO: 180 |
| Alternative intron chr17: 50658497-50659181 | SEQ ID NO: 181 |

ABCC5

| | |
|---|---|
| Canonical exon chr3: 183971563-183971919 | SEQ ID NO: 182 |
| Alternative exon chr3: 183971563-183972034 | SEQ ID NO: 183 |
| Canonical intron chr3: 183971920-183977516 | SEQ ID NO: 184 |
| Alternative intron chr3: 183972035-183977516 | SEQ ID NO: 185 |

ABCC8

| | |
|---|---|
| Canonical exon chr11: 17406888-17407129 | SEQ ID NO: 186 |
| Alternative exon chr11: 17406888-17407209 | SEQ ID NO: 187 |
| Canonical intron chr11: 17407130-17407353 | SEQ ID NO: 188 |
| Alternative intron chr11: 17407210-17407353 | SEQ ID NO: 189 |

ABCD1

| | |
|---|---|
| Canonical exon chrX: 153736345-153736513 | SEQ ID NO: 190 |
| Alternative exon chrX: 153736345-153736911 | SEQ ID NO: 191 |
| Canonical intron chrX: 153736514-153737156 | SEQ ID NO: 192 |
| Alternative intron chrX: 153736912-153737156 | SEQ ID NO: 193 |

ABR

| | |
|---|---|
| Canonical exon chr17: 1007165-1007312 | SEQ ID NO: 194 |
| Alternative exon chr17: 1006775-1007312 | SEQ ID NO: 195 |
| Alternative exon chr17: 1006840-1007312 | SEQ ID NO: 196 |
| Alternative exon chr17: 1006905-1007312 | SEQ ID NO: 197 |
| Canonical intron chr17: 1006170-1007164 | SEQ ID NO: 198 |
| Alternative intron chr17: 1006170-1006774 | SEQ ID NO: 199 |
| Alternative intron chr17: 1006170-1006839 | SEQ ID NO: 200 |
| Alternative intron chr17: 1006170-1006904 | SEQ ID NO: 201 |

ACAD9

| | |
|---|---|
| Canonical exon chr3: 128879620-128879841 | SEQ ID NO: 202 |
| Canonical exon chr3: 128879629-128879841 | SEQ ID NO: 203 |
| Alternative exon chr3: 128879620-128880017 | SEQ ID NO: 204 |
| Alternative exon chr3: 128879629-128880017 | SEQ ID NO: 205 |
| Canonical intron chr3: 128879842-128884652 | SEQ ID NO: 206 |
| Alternative intron chr3: 128880018-128884652 | SEQ ID NO: 207 |

ACAP1

| | |
|---|---|
| Canonical exon | SEQ ID NO: 208 |
| Canonical intron | SEQ ID NO: 209 |
| Alternative exon | SEQ ID NO: 210 |
| Alternative intron | SEQ ID NO: 211 |

ACOX2

| | |
|---|---|
| Canonical exon chr3: 58534360-58534522 | SEQ ID NO: 212 |
| Alternative exon chr3: 58534360-58534608 | SEQ ID NO: 213 |
| Canonical intron chr3: 58534523-58534946 | SEQ ID NO: 214 |
| Alternative intron chr3: 58534609-58534946 | SEQ ID NO: 215 |

ACSF3

| | |
|---|---|
| Canonical exon chr16: 89145267-89145400 | SEQ ID NO: 216 |
| Canonical exon chr16: 89145267-89145401 | SEQ ID NO: 217 |
| Alternative exon chr16: 89145214-89145400 | SEQ ID NO: 218 |
| Alternative exon chr16: 89145214-89145401 | SEQ ID NO: 219 |
| Canonical intron chr16: 89133263-89145266 | SEQ ID NO: 220 |
| Alternative intron chr16: 89133263-89145213 | SEQ ID NO: 221 |

ACTN4

| | |
|---|---|
| Canonical exon chr19: 38727946-38728026 | SEQ ID NO: 222 |
| Alternative exon chr19: 38727946-38728381 | SEQ ID NO: 223 |
| Canonical intron chr19: 38728027-38728995 | SEQ ID NO: 224 |
| Alternative intron chr19: 38728382-38728995 | SEQ ID NO: 225 |

ADAM17

| | |
|---|---|
| Canonical exon chr2: 9526111-9526244 | SEQ ID NO: 226 |
| Alternative exon chr2: 9526111-9526264 | SEQ ID NO: 227 |
| Canonical intron chr2: 9526245-9527785 | SEQ ID NO: 228 |
| Alternative intron chr2 9526265 9527785 | SEQ ID NO: 229 |

ADAMTS13

| | |
|---|---|
| Canonical exon | SEQ ID NO: 230 |
| Canonical exon | SEQ ID NO: 231 |
| Canonical intron | SEQ ID NO: 232 |
| Canonical intron | SEQ ID NO: 233 |
| Alternative exon | SEQ ID NO: 234 |
| Alternative exon | SEQ ID NO: 235 |
| Alternative exon | SEQ ID NO: 236 |
| Alternative intron | SEQ ID NO: 237 |
| Alternative intron | SEQ ID NO: 238 |

AKR1E2

| | |
|---|---|
| Canonical exon chr10: 4847148-4847230 | SEQ ID NO: 239 |
| Alternative exon chr10: 4847148-4847299 | SEQ ID NO: 240 |
| Canonical intron chr10: 4847231-4847487 | SEQ ID NO: 241 |
| Alternative intron chr10: 4847300-4847487 | SEQ ID NO: 242 |

ALAD

| | |
|---|---|
| Canonical exon chr9: 113392119-113392169 | SEQ ID NO: 243 |
| Canonical exon chr9: 113392132-113392169 | SEQ ID NO: 244 |
| Alternative exon chr9: 113392119-113392335 | SEQ ID NO: 245 |
| Alternative exon chr9: 113392132-113392335 | SEQ ID NO: 246 |
| Canonical intron chr9: 113392170-113393446 | SEQ ID NO: 247 |
| Alternative intron chr9: 113392336-113393446 | SEQ ID NO: 248 |

ALG3

| | |
|---|---|
| Canonical exon | SEQ ID NO: 249 |
| Canonical exon | SEQ ID NO: 250 |
| Canonical intron | SEQ ID NO: 251 |
| Canonical intron | SEQ ID NO: 252 |
| Alternative exon | SEQ ID NO: 253 |
| Alternative exon | SEQ ID NO: 254 |
| Alternative intron | SEQ ID NO: 255 |

ANKRD29

| | |
|---|---|
| Canonical exon chr18: 23619531-23619629 | SEQ ID NO: 256 |
| Alternative exon chr18: 23619395-23619629 | SEQ ID NO: 257 |
| Canonical intron chr18: 23617828-23619530 | SEQ ID NO: 258 |
| Alternative intron chr18: 23617828-23619394 | SEQ ID NO: 259 |

ANKS3

| | |
|---|---|
| Canonical exon | SEQ ID NO: 260 |
| Canonical intron | SEQ ID NO: 261 |
| Alternative exon | SEQ ID NO: 262 |
| Alternative intron | SEQ ID NO: 263 |

ANO4

| | |
|---|---|
| Canonical exon chr12: 100974845-100974889 | SEQ ID NO: 264 |
| Alternative exon chr12: 100974783-100974889 | SEQ ID NO: 265 |
| Canonical intron chr12: 100971407-100974844 | SEQ ID NO: 266 |
| Alternative intron chr12: 100971407-100974782 | SEQ ID NO: 267 |

AP3B2

| | |
|---|---|
| Canonical exon chr15: 82663127-82663233 | SEQ ID NO: 268 |
| Alternative exon chr15: 82663127-82663295 | SEQ ID NO: 269 |
| Canonical intron chr15: 82663234-82663559 | SEQ ID NO: 270 |
| Alternative intron chr15: 82663296-82663559 | SEQ ID NO: 271 |

AP3M1

| | |
|---|---|
| Canonical exon chr10: 74126148-74126355 | SEQ ID NO: 272 |
| Alternative exon chr10: 74126126-74126355 | SEQ ID NO: 273 |
| Canonical intron chr10: 74124525-74126147 | SEQ ID NO: 274 |
| Alternative intron chr10: 74124525-74126125 | SEQ ID NO: 275 |

AP5Z1

| | |
|---|---|
| Canonical exon chr7: 4787634-4787776 | SEQ ID NO: 276 |
| Alternative exon chr7: 4787596-4787776 | SEQ ID NO: 277 |
| Canonical intron chr7: 4786429-4787633 | SEQ ID NO: 278 |
| Alternative intron chr7: 4786429-4787595 | SEQ ID NO: 279 |

ARNTL

| | |
|---|---|
| Canonical exon chr11: 13376640-13376728 | SEQ ID NO: 280 |
| Alternative exon chr11: 13376545-13376728 | SEQ ID NO: 281 |
| Canonical intron chr11: 13375763-13376639 | SEQ ID NO: 282 |
| Alternative intron chr11: 13375763-13376544 | SEQ ID NO: 283 |

ASAP3

| | |
|---|---|
| Canonical exon | SEQ ID NO: 284 |
| Canonical intron | SEQ ID NO: 285 |
| Alternative exon | SEQ ID NO: 286 |
| Alternative intron | SEQ ID NO: 287 |

ATRX

| | |
|---|---|
| Canonical exon chrX: 77785982-77786208 | SEQ ID NO: 288 |
| Canonical exon chrX: 77785982-77786216 | SEQ ID NO: 289 |
| Canonical exon chrX: 77785982-77786220 | SEQ ID NO: 290 |
| Canonical exon chrX: 77785982-77786225 | SEQ ID NO: 291 |
| Alternative exon chrX: 77785845-77786208 | SEQ ID NO: 292 |
| Alternative exon chrX:77785845-77786216 | SEQ ID NO: 293 |
| Alternative exon chrX: 77785845-77786220 | SEQ ID NO: 294 |
| Alternative exon chrX: 77785845-77786225 | SEQ ID NO: 295 |
| Canonical intron chrX: 77717244-77785981 | SEQ ID NO: 296 |
| Alternative intron chrX: 77717244-77785844 | SEQ ID NO: 297 |

BBS2

| | |
|---|---|
| Canonical exon chr16: 56498437-56498568 | SEQ ID NO: 298 |
| Alternative exon chr16: 56498437-56498693 | SEQ ID NO: 299 |
| Canonical intron chr16: 56498569-56499777 | SEQ ID NO: 300 |
| Alternative intron chr16: 56498694-56499777 | SEQ ID NO: 301 |

BBS4

| | |
|---|---|
| Canonical exon chr15: 72686179-72686251 | SEQ ID NO: 302 |
| Canonical exon chr15: 72686207-72686251 | SEQ ID NO: 303 |
| Canonical exon chr15: 72686237-72686251 | SEQ ID NO: 304 |
| Alternative exon chr15: 72686179-72686564 | SEQ ID NO: 305 |
| Alternative exon chr15:72686207-72686564 | SEQ ID NO: 306 |
| Alternative exon chr15: 72686237-72686564 | SEQ ID NO: 307 |
| Canonical intron chr15: 72686252-72695176 | SEQ ID NO: 308 |
| Alternative intron chr15: 72686565-72695176 | SEQ ID NO: 309 |

BRD9

| | |
|---|---|
| Canonical exon chr5: 889587-889647 | SEQ ID NO: 310 |
| Alternative exon chr5: 889587-889766 | SEQ ID NO: 311 |
| Canonical intron chr5: 889648-891154 | SEQ ID NO: 312 |
| Alternative intron chr5: 889767-891154 | SEQ ID NO: 313 |

CALM3

| | |
|---|---|
| Canonical exon chr19: 46608924-46608981 | SEQ ID NO: 314 |
| Alternative exon chr19: 46608482-46608981 | SEQ ID NO: 315 |
| Canonical intron chr19: 46608328-46608923 | SEQ ID NO: 316 |
| Alternative intron chr19: 46608328-46608481 | SEQ ID NO: 317 |

CARS1

| | |
|---|---|
| Canonical exon | SEQ ID NO: 318 |
| Canonical intron | SEQ ID NO: 319 |
| Alternative exon | SEQ ID NO: 320 |
| Alternative intron | SEQ ID NO: 321 |

CASP9

| | |
|---|---|
| Canonical exon chr1: 15506899-15507075 | SEQ ID NO: 322 |
| Alternative exon chr1: 15506899-15507133 | SEQ ID NO: 323 |
| Canonical intron chr1: 15507076-15507872 | SEQ ID NO: 324 |
| Alternative intron chr1: 15507134-15507872 | SEQ ID NO: 325 |

CATSPER1

| | |
|---|---|
| Canonical exon | SEQ ID NO: 326 |
| Canonical intron | SEQ ID NO: 327 |
| Alternative exon | SEQ ID NO: 328 |
| Alternative intron | SEQ ID NO: 329 |

CCDC40

| | |
|---|---|
| Canonical exon | SEQ ID NO: 330 |
| Canonical intron | SEQ ID NO: 331 |
| Alternative exon | SEQ ID NO: 332 |
| Alternative intron | SEQ ID NO: 333 |

CCDC88B

| | |
|---|---|
| Canonical exon chr11: 64342522-64342680 | SEQ ID NO: 334 |
| Alternative exon chr11: 64342522-64342807 | SEQ ID NO: 335 |
| Canonical intron chr11: 64342681-64343178 | SEQ ID NO: 336 |
| Alternative intron chr11: 64342808-64343178 | SEQ ID NO: 337 |

CD4

| | |
|---|---|
| Canonical exon chr12: 6818421-6818542 | SEQ ID NO: 338 |
| Alternative exon chr12: 6818421-6818650 | SEQ ID NO: 339 |
| Canonical intron chr12: 6818543-6818846 | SEQ ID NO: 340 |
| Alternative intron chr12: 6818651-6818846 | SEQ ID NO: 341 |

CD58

| | |
|---|---|
| Canonical exon chr1: 116518700-116519267 | SEQ ID NO: 342 |
| Canonical exon chr1: 116519231-116519267 | SEQ ID NO: 343 |
| Alternative exon chr1: 116518700-116519302 | SEQ ID NO: 344 |
| Alternative exon chr1: 116519231-116519302 | SEQ ID NO: 345 |
| Canonical intron chr1: 116519268-116521905 | SEQ ID NO: 346 |
| Alternative intron chr1: 116519303-116521905 | SEQ ID NO: 347 |

CES2

| | |
|---|---|
| Canonical exon | SEQ ID NO: 348 |
| Canonical exon | SEQ ID NO: 349 |
| Canonical exon | SEQ ID NO: 350 |
| Canonical intron | SEQ ID NO: 351 |
| Alternative exon | SEQ ID NO: 352 |
| Alternative exon | SEQ ID NO: 353 |
| Alternative exon | SEQ ID NO: 354 |
| Alternative exon | SEQ ID NO: 355 |
| Alternative exon | SEQ ID NO: 356 |
| Alternative exon | SEQ ID NO: 357 |
| Alternative intron | SEQ ID NO: 358 |
| Alternative intron | SEQ ID NO: 359 |

CHFR

| | |
|---|---|
| Canonical exon chr12: 132848085-132848155 | SEQ ID NO: 360 |
| Alternative exon chr12: 132848085-132848257 | SEQ ID NO: 361 |
| Canonical intron chr12: 132848156-132848640 | SEQ ID NO: 362 |
| Alternative intron chr12: 132848258-132848640 | SEQ ID NO: 363 |

CLCN2

| | |
|---|---|
| Canonical exon chr3: 184354548-184354607 | SEQ ID NO: 364 |
| Canonical exon chr3: 184354548-184354658 | SEQ ID NO: 365 |
| Alternative exon chr3: 184354548-184354714 | SEQ ID NO: 366 |
| Canonical intron chr3: 184354608-184354903 | SEQ ID NO: 367 |
| Canonical intron chr3: 184354659-184354903 | SEQ ID NO: 368 |
| Alternative intron chr3: 184354715-184354903 | SEQ ID NO: 369 |

CNOT3

| | |
|---|---|
| Canonical exon chr19: 54145598-54145698 | SEQ ID NO: 370 |
| Canonical exon chr19: 54145598-54145718 | SEQ ID NO: 371 |
| Canonical exon chr19: 54145598-54145817 | SEQ ID NO: 372 |
| Alternative exon chr19: 54145542-54145698 | SEQ ID NO: 373 |
| Alternative exon chr19: 54145542-54145718 | SEQ ID NO: 374 |
| Alternative exon chr19: 54145542-54145817 | SEQ ID NO: 375 |
| Canonical intron chr19: 54144333-54145597 | SEQ ID NO: 376 |
| Alternative intron chr19: 54144333-54145541 | SEQ ID NO: 377 |

CNTROB

| | |
|---|---|
| Canonical exon chr17: 7936366-7936482 | SEQ ID NO: 378 |
| Alternative exon chr17: 7936238-7936482 | SEQ ID NO: 379 |
| Canonical intron chr17: 7935146-7936365 | SEQ ID NO: 380 |
| Alternative intron chr17: 7935146-7936237 | SEQ ID NO: 381 |

COL5A3

| | |
|---|---|
| Canonical exon | SEQ ID NO: 382 |
| Canonical intron | SEQ ID NO: 383 |
| Alternative exon | SEQ ID NO: 384 |
| Alternative intron | SEQ ID NO: 385 |

CPSF1

| | |
|---|---|
| Canonical exon | SEQ ID NO: 386 |
| Canonical intron | SEQ ID NO: 387 |
| Alternative exon | SEQ ID NO: 388 |
| Alternative intron | SEQ ID NO: 389 |

CSPP1

| | |
|---|---|
| Canonical exon chr8: 67064368-67064496 | SEQ ID NO: 390 |
| Alternative exon chr8: 67064368-67064538 | SEQ ID NO: 391 |
| Canonical intron chr8: 67064497-67074242 | SEQ ID NO: 392 |
| Alternative intron chr8: 67064539-67074242 | SEQ ID NO: 393 |

CTF1

| | |
|---|---|
| Canonical exon chr16: 30899415-30899533 | SEQ ID NO: 394 |
| Canonical exon chr16: 30899418-30899533 | SEQ ID NO: 395 |
| Alternative exon chr16: 30899307-30899533 | SEQ ID NO: 396 |
| Canonical intron chr16: 30896669-30899414 | SEQ ID NO: 397 |
| Canonical intron chr16: 30896669-30899417 | SEQ ID NO: 398 |
| Alternative intron chr16: 30896669-30899306 | SEQ ID NO: 399 |

CTH

| | |
|---|---|
| Canonical exon chr1: 70411218-70411583 | SEQ ID NO: 400 |
| Canonical exon chr1: 70411268-70411583 | SEQ ID NO: 401 |
| Canonical exon chr1: 70411383-70411583 | SEQ ID NO: 402 |
| Alternative exon chr1: 70411218-70411656 | SEQ ID NO: 403 |
| Alternative exon chr1: 70411268-70411656 | SEQ ID NO: 404 |
| Alternative exon chr1: 70411383-70411656 | SEQ ID NO: 405 |
| Canonical intron chr1: 70411584-70415955 | SEQ ID NO: 406 |
| Alternative intron chr1: 70411657-70415955 | SEQ ID NO: 407 |

CYP3A5

| | |
|---|---|
| Canonical exon chr7: 99666601-99666689 | SEQ ID NO: 408 |
| Alternative exon chr7: 99666601-99666810 | SEQ ID NO: 409 |
| Canonical intron chr7: 99666690-99666951 | SEQ ID NO: 410 |
| Alternative intron chr7: 99666811-99666951 | SEQ ID NO: 411 |

DAB2IP

| | |
|---|---|
| Canonical exon chr9: 121678678-121678781 | SEQ ID NO: 412 |
| Alternative exon chr9: 121678678-121678917 | SEQ ID NO: 413 |
| Canonical intron chr9: 121678782-121699324 | SEQ ID NO: 414 |
| Alternative intron chr9: 121678918-121699324 | SEQ ID NO: 415 |

DBN1

| | |
|---|---|
| Canonical exon chr5: 177468108-177468217 | SEQ ID NO: 416 |
| Canonical exon chr5: 177468108-177468220 | SEQ ID NO: 417 |
| Alternative exon chr5: 177467996-177468217 | SEQ ID NO: 418 |
| Alternative exon chr5: 177467996-177468220 | SEQ ID NO: 419 |
| Alternative exon chr5: 177468052-177468217 | SEQ ID NO: 420 |
| Alternative exon chr5: 177468052-177468220 | SEQ ID NO: 421 |
| Canonical intron chr5: 177467818-177468107 | SEQ ID NO: 422 |
| Alternative intron chr5: 177467818-177467995 | SEQ ID NO: 423 |
| Alternative intron chr5: 177467818-177468051 | SEQ ID NO: 424 |

DDX51

| | |
|---|---|
| Canonical exon chr12: 132141275-132141420 | SEQ ID NO: 425 |
| Alternative exon chr12: 132141103-132141420 | SEQ ID NO: 426 |
| Canonical intron chr12: 132141021-132141274 | SEQ ID NO: 427 |
| Alternative intron chr12: 132141021-132141102 | SEQ ID NO: 428 |

DEAF1

| | |
|---|---|
| Canonical exon chr11: 694759-695532 | SEQ ID NO: 429 |
| Alternative exon chr11: 694679-695532 | SEQ ID NO: 430 |
| Canonical intron chr11: 691599-694758 | SEQ ID NO: 431 |
| Alternative intron chr11: 691599-694678 | SEQ ID NO: 432 |

DCKE

| | |
|---|---|
| Canonical exon chr17: 56856512-56856625 | SEQ ID NO: 433 |
| Alternative exon chr17: 56856512-56856665 | SEQ ID NO: 434 |
| Canonical intron chr17: 56856626-56858593 | SEQ ID NO: 435 |
| Alternative intron chr17: 56856666-56858593 | SEQ ID NO: 436 |

DHDDS

| | |
|---|---|
| Canonical exon | SEQ ID NO: 437 |
| Canonical exon | SEQ ID NO: 438 |
| Canonical exon | SEQ ID NO: 439 |
| Canonical intron | SEQ ID NO: 440 |
| Alternative exon | SEQ ID NO: 441 |
| Alternative exon | SEQ ID NO: 442 |
| Alternative exon | SEQ ID NO: 443 |
| Alternative intron | SEQ ID NO: 444 |

DMKN

| | |
|---|---|
| Canonical exon chr19: 35498716-35498763 | SEQ ID NO: 445 |
| Alternative exon chr19: 35498598-35498763 | SEQ ID NO: 446 |
| Canonical intron chr19: 35497539-35498715 | SEQ ID NO: 447 |
| Alternative intron chr19: 35497539-35498597 | SEQ ID NO: 448 |

DNHD1

| | |
|---|---|
| Canonical exon chr canonical_exon_chr11_6566241_6566393 | SEQ ID NO: 449 |
| Alternative exon chr nmd_exon_chr11_6566101_6566393 | SEQ ID NO: 450 |
| Canonical intron chr canonical_intron_chr11_6565992_6566240 | SEQ ID NO: 451 |
| Alternative intron chr nmd_intron_chr11_6565992_6566100 | SEQ ID NO: 452 |

DOCK8

| | |
|---|---|
| Canonical exon chr9: 214865-215029 | SEQ ID NO: 453 |
| Alternative exon chr9: 214865-215416 | SEQ ID NO: 454 |
| Canonical intron chr9: 215030-271626 | SEQ ID NO: 455 |
| Alternative intron chr9: 215417-271626 | SEQ ID NO: 456 |

DOK5

| | |
|---|---|
| Canonical exon chr20: 54475593-54476012 | SEQ ID NO: 457 |
| Alternative exon chr20: 54475593-54476185 | SEQ ID NO: 458 |
| Canonical intron chr20: 54476013-54554932 | SEQ ID NO: 459 |
| Alternative intron chr20: 54476186-54554932 | SEQ ID NO: 460 |

EIF2B5

| | |
|---|---|
| Canonical exon chr3: 184140080-184140157 | SEQ ID NO: 461 |
| Alternative exon chr3: 184140080-184140221 | SEQ ID NO: 462 |
| Canonical intron chr3: 184140158-184140417 | SEQ ID NO: 463 |
| Alternative intron chr3: 184140222-184140417 | SEQ ID NO: 464 |

ELAC2

| | |
|---|---|
| Canonical exon chr17: 13017703-13017966 | SEQ ID NO: 465 |
| Canonical exon chr17: 13017703-13017999 | SEQ ID NO: 466 |
| Canonical exon chr17: 13017703-13018027 | SEQ ID NO: 467 |
| Alternative exon chr17: 13017567-13017966 | SEQ ID NO: 468 |
| Alternative exon chr17: 13017567-13017999 | SEQ ID NO: 469 |
| Alternative exon chr17: 13017567-13018027 | SEQ ID NO: 470 |
| Canonical intron chr17: 13017122-13017702 | SEQ ID NO: 471 |
| Alternative intron chr17: 13017122-13017566 | SEQ ID NO: 472 |

ELP1

| | |
|---|---|
| Canonical exon chr9: 108896953-108897038 | SEQ ID NO: 473 |
| Alternative exon chr9:108896891-108897038 | SEQ ID NO: 474 |
| Canonical intron chr9: 108896645-108896952 | SEQ ID NO: 475 |
| Alternative intron chr9:108896645-108896890 | SEQ ID NO: 476 |

ENTPD4

| | |
|---|---|
| Canonical exon chr8: 23434317-23434478 | SEQ ID NO: 477 |
| Alternative exon chr8: 23434317-23434681 | SEQ ID NO: 478 |
| Canonical intron chr8: 23434479-23435391 | SEQ ID NO: 479 |
| Alternative intron chr8: 23434682-23435391 | SEQ ID NO: 480 |

ESRRA

| | |
|---|---|
| Canonical exon chr11: 64313951-64314067 | SEQ ID NO: 481 |
| Alternative exon chr11: 64313854-64314067 | SEQ ID NO: 482 |
| Canonical intron chr11: 64307505-64313950 | SEQ ID NO: 483 |
| Alternative intron chr11: 64307505-64313853 | SEQ ID NO: 484 |

ESS2

| | |
|---|---|
| Canonical exon chr22: 19138215-19138317 | SEQ ID NO: 485 |
| Alternative exon chr22: 19138215-19138479 | SEQ ID NO: 486 |
| Canonical intron chr22: 19138318-19139158 | SEQ ID NO: 487 |
| Alternative intron chr22: 19138480-19139158 | SEQ ID NO: 488 |

ETAA1

| | |
|---|---|
| Canonical exon chr2: 67405236-67405335 | SEQ ID NO: 489 |
| Alternative exon chr2: 67404997-67405335 | SEQ ID NO: 490 |
| Canonical intron chr2: 67402975-67405235 | SEQ ID NO: 491 |
| Alternative intron chr2: 67402975-67404996 | SEQ ID NO: 492 |

ETHE1

| | |
|---|---|
| Canonical exon chr19: 43527097-43527201 | SEQ ID NO: 493 |
| Alternative exon chr19: 43526937-43527201 | SEQ ID NO: 494 |
| Canonical intron chr19: 43526660-43527096 | SEQ ID NO: 495 |
| Alternative intron chr19: 43526660-43526936 | SEQ ID NO: 496 |

FANCA

| | |
|---|---|
| Canonical exon chr16: 89739994-89740078 | SEQ ID NO: 497 |
| Canonical exon chr16: 89739994-89740099 | SEQ ID NO: 498 |
| Alternative exon chr16: 89739706-89740078 | SEQ ID NO: 499 |
| Alternative exon chr16: 89739706-89740099 | SEQ ID NO: 500 |
| Canonical intron chr16: 89739554-89739993 | SEQ ID NO: 501 |
| Alternative intron chr16: 89739554-89739705 | SEQ ID NO: 502 |

FASTK

| | |
|---|---|
| Canonical exon | SEQ ID NO: 503 |
| Canonical exon | SEQ ID NO: 504 |
| Canonical exon | SEQ ID NO: 505 |
| Canonical exon | SEQ ID NO: 506 |
| Canonical intron | SEQ ID NO: 507 |
| Alternative exon | SEQ ID NO: 508 |
| Alternative exon | SEQ ID NO: 509 |
| Alternative exon | SEQ ID NO: 510 |
| Alternative exon | SEQ ID NO: 511 |
| Alternative exon | SEQ ID NO: 512 |
| Alternative exon | SEQ ID NO: 513 |
| Alternative exon | SEQ ID NO: 514 |
| Alternative exon | SEQ ID NO: 515 |
| Alternative intron | SEQ ID NO: 516 |
| Alternative intron | SEQ ID NO: 517 |

FASTKD3

| | |
|---|---|
| Canonical exon chr5: 7861583-7861652 | SEQ ID NO: 518 |
| Alternative exon chr5: 7861583-7861773 | SEQ ID NO: 519 |
| Canonical intron chr5: 7861653-7862822 | SEQ ID NO: 520 |
| Alternative intron chr5: 7861774-7862822 | SEQ ID NO: 521 |

FPGS

| | |
|---|---|
| Canonical exon chr9: 127807586-127807688 | SEQ ID NO: 522 |
| Alternative exon chr9: 127807586-127807775 | SEQ ID NO: 523 |
| Canonical intron chr9: 127807689-127808233 | SEQ ID NO: 524 |
| Alternative intron chr9: 127807776-127808233 | SEQ ID NO: 525 |

FREM1

| | |
|---|---|
| Canonical exon | SEQ ID NO: 526 |
| Canonical intron | SEQ ID NO: 527 |
| Alternative exon | SEQ ID NO: 528 |
| Alternative exon | SEQ ID NO: 529 |
| Alternative intron | SEQ ID NO: 530 |
| Alternative intron | SEQ ID NO: 531 |

GAS8

| | |
|---|---|
| Canonical exon chr16: 90031299-90031496 | SEQ ID NO: 532 |
| Canonical exon chr16: 90035633-90035687 | SEQ ID NO: 533 |
| Alternative exon chr16: 90031190-90031496 | SEQ ID NO: 534 |
| Alternative exon chr16: 90035633-90035879 | SEQ ID NO: 535 |
| Canonical intron chr16: 90027723-90031298 | SEQ ID NO: 536 |
| Canonical intron chr16: 90035688-90036380 | SEQ ID NO: 537 |
| Alternative intron chr16: 90027723-90031189 | SEQ ID NO: 538 |
| Alternative intron chr16: 90035880-90036380 | SEQ ID NO: 539 |

GCK

| | |
|---|---|
| Canonical exon chr7: 44146463-44146618 | SEQ ID NO: 540 |
| Alternative exon chr7: 44146074-44146618 | SEQ ID NO: 541 |
| Canonical intron chr7: 44145731-44146462 | SEQ ID NO: 542 |
| Alternative intron chr7: 44145731-44146073 | SEQ ID NO: 543 |

GGA1

| | |
|---|---|
| Canonical exon | SEQ ID NO: 544 |
| Canonical exon | SEQ ID NO: 545 |
| Canonical exon | SEQ ID NO: 546 |
| Canonical exon | SEQ ID NO: 547 |
| Canonical intron | SEQ ID NO: 548 |
| Alternative exon | SEQ ID NO: 549 |
| Alternative exon | SEQ ID NO: 550 |
| Alternative exon | SEQ ID NO: 551 |
| Alternative exon | SEQ ID NO: 552 |
| Alternative intron | SEQ ID NO: 553 |

GGA3

| | |
|---|---|
| Canonical exon chr17: 75243063-75243166 | SEQ ID NO: 554 |
| Alternative exon chr17: 75243063-75243258 | SEQ ID NO: 555 |
| Canonical intron chr17: 75243167-75243446 | SEQ ID NO: 556 |
| Alternative intron chr17: 75243259-75243446 | SEQ ID NO: 557 |

GGT1

| | |
|---|---|
| Canonical exon chr22: 24627432-24627619 | SEQ ID NO: 558 |
| Alternative exon chr22: 24627410-24627619 | SEQ ID NO: 559 |
| Canonical intron chr22: 24623917-24627431 | SEQ ID NO: 560 |
| Alternative intron chr22: 24623917-24627409 | SEQ ID NO: 561 |

GLMN

| | |
|---|---|
| Canonical exon chr1: 92266419-92266492 | SEQ ID NO: 562 |
| Canonical exon chr1: 92290198-92290306 | SEQ ID NO: 563 |
| Alternative exon chr1: 92266419-92266593 | SEQ ID NO: 564 |
| Alternative exon chr1: 92290198-92290330 | SEQ ID NO: 565 |
| Canonical intron chr1: 92266493-92266699 | SEQ ID NO: 566 |
| Canonical intron chr1: 92290307-92291417 | SEQ ID NO: 567 |
| Alternative intron chr1: 92266594-92266699 | SEQ ID NO: 568 |
| Alternative intron chr1: 92290331-92291417 | SEQ ID NO: 569 |

GON4L

| | |
|---|---|
| Canonical exon chr1: 155775002-155775173 | SEQ ID NO: 570 |
| Alternative exon chr1: 155774963-155775173 | SEQ ID NO: 571 |
| Canonical intron chr1: 155773211-155775001 | SEQ ID NO: 572 |
| Alternative intron chr1: 155773211-155774962 | SEQ ID NO: 573 |

GRB14

| | |
|---|---|
| Canonical exon chr2: 164508455-164508550 | SEQ ID NO: 574 |
| Alternative exon chr2: 164508455-164508603 | SEQ ID NO: 575 |
| Canonical intron chr2: 164508551-164508741 | SEQ ID NO: 576 |
| Alternative intron chr2: 164508604-164508741 | SEQ ID NO: 577 |

HDAC3

| | |
|---|---|
| Canonical exon chr5: 141628114-141628187 | SEQ ID NO: 578 |
| Alternative exon chr5: 141628114-141628212 | SEQ ID NO: 579 |
| Canonical intron chr5: 141628188-141628558 | SEQ ID NO: 580 |
| Alternative intron chr5: 141628213-141628558 | SEQ ID NO: 581 |

HELQ

| | |
|---|---|
| Canonical exon | SEQ ID NO: 582 |
| Canonical intron | SEQ ID NO: 583 |
| Alternative exon | SEQ ID NO: 584 |
| Alternative intron | SEQ ID NO: 585 |

HP1BP3

| | |
|---|---|
| Canonical exon chr1: 20749723-20749882 | SEQ ID NO: 586 |
| Alternative exon chr1: 20749723-20749975 | SEQ ID NO: 587 |
| Canonical intron chr1: 20749883-20757165 | SEQ ID NO: 588 |
| Alternative intron chr1: 20749976-20757165 | SEQ ID NO: 589 |

HPS1

| | |
|---|---|
| Canonical exon chr10: 98433983-98434091 | SEQ ID NO: 590 |
| Alternative exon chr10: 98433940-98434091 | SEQ ID NO: 591 |
| Canonical intron chr10: 98431292-98433982 | SEQ ID NO: 592 |
| Alternative intron chr10: 98431292-98433939 | SEQ ID NO: 593 |

HPS4

| | |
|---|---|
| Canonical exon chr22: 26470719-26470813 | SEQ ID NO: 594 |
| Alternative exon chr22: 26470719-26470922 | SEQ ID NO: 595 |
| Canonical intron chr22: 26470814-26472301 | SEQ ID NO: 596 |
| Alternative intron chr22: 26470923-26472301 | SEQ ID NO: 597 |

IDUA

| | |
|---|---|
| Canonical exon chr4: 1001679-1001715 | SEQ ID NO: 598 |
| Canonical exon chr4: 1001679-1001881 | SEQ ID NO: 599 |
| Alternative exon chr4: 1001651-1001715 | SEQ ID NO: 600 |
| Alternative exon chr4: 1001651-1001881 | SEQ ID NO: 601 |
| Canonical intron chr4: 1001564-1001678 | SEQ ID NO: 602 |
| Alternative intron chr4: 1001564-1001650 | SEQ ID NO: 603 |

IFT122

| | |
|---|---|
| Canonical exon chr3: 129515488-129515599 | SEQ ID NO: 604 |
| Alternative exon chr3: 129515400-129515599 | SEQ ID NO: 605 |
| Canonical intron chr3: 129514555-129515487 | SEQ ID NO: 606 |
| Alternative intron chr3: 129514555-129515399 | SEQ ID NO: 607 |

IL17RC

| | |
|---|---|
| Canonical exon chr3: 9923881-9924020 | SEQ ID NO: 608 |
| Alternative exon chr3: 9923881-9924137 | SEQ ID NO: 609 |
| Canonical intron chr3: 9924021-9924231 | SEQ ID NO: 610 |
| Alternative intron chr3: 9924138-9924231 | SEQ ID NO: 611 |

IMPDH1

| | |
|---|---|
| Canonical exon chr7: 128400095-128400182 | SEQ ID NO: 612 |
| Canonical exon chr7: 128400135-128400182 | SEQ ID NO: 613 |
| Alternative exon chr7: 128400095-128400204 | SEQ ID NO: 614 |
| Alternative exon chr7: 128400135-128400204 | SEQ ID NO: 615 |
| Canonical intron chr7: 128400183-128400332 | SEQ ID NO: 616 |
| Alternative intron chr7: 128400205-128400332 | SEQ ID NO: 617 |

INVS

| | |
|---|---|
| Canonical exon chr9: 100292326-100292436 | SEQ ID NO: 618 |
| Alternative exon chr9: 100292326-100292873 | SEQ ID NO: 619 |
| Canonical intron chr9: 100292437-100292946 | SEQ ID NO: 620 |
| Alternative intron chr9: 100292874-100292946 | SEQ ID NO: 621 |

IRF3

| | |
|---|---|
| Canonical exon chr19: 49661948-49662328 | SEQ ID NO: 622 |
| Alternative exon chr19: 49661450-49662328 | SEQ ID NO: 623 |
| Canonical intron chr19: 49660829-49661947 | SEQ ID NO: 624 |
| Canonical intron chr19: 49660845-49661947 | SEQ ID NO: 625 |
| Alternative intron chr19: 49660829-49661449 | SEQ ID NO: 626 |
| Alternative intron chr19: 49660845-49661449 | SEQ ID NO: 627 |

KCTD3

| | |
|---|---|
| Canonical exon chr1: 215578001-215578081 | SEQ ID NO: 628 |
| Alternative exon chr1: 215577929-215578081 | SEQ ID NO: 629 |
| Canonical intron chr1: 215577729-215578000 | SEQ ID NO: 630 |
| Alternative intron chr1: 215577729-215577928 | SEQ ID NO: 631 |

KLKB1

| | |
|---|---|
| Canonical exon chr4: 186250243-186250402 | SEQ ID NO: 632 |
| Alternative exon chr4: 186250243-186250433 | SEQ ID NO: 633 |
| Canonical intron chr4: 186250403-186251218 | SEQ ID NO: 634 |
| Alternative intron chr4: 186250434-186251218 | SEQ ID NO: 635 |

KYAT1

| | |
|---|---|
| Canonical exon chr9: 128835997-128836073 | SEQ ID NO: 636 |
| Canonical exon chr9: 128836009-128836073 | SEQ ID NO: 637 |
| Alternative exon chr9: 128835997-128836105 | SEQ ID NO: 638 |
| Alternative exon chr9: 128836009-128836105 | SEQ ID NO: 639 |
| Canonical intron chr9: 128836074-128836801 | SEQ ID NO: 640 |
| Alternative intron chr9: 128836106-128836801 | SEQ ID NO: 641 |

LAMC3

| | |
|---|---|
| Canonical exon chr9: 131085524-131085723 | SEQ ID NO: 642 |
| Canonical exon chr9: 131085608-131085723 | SEQ ID NO: 643 |
| Alternative exon chr9: 131085524-131085871 | SEQ ID NO: 644 |
| Alternative exon chr9: 131085608-131085871 | SEQ ID NO: 645 |
| Canonical intron chr9: 131085724-131087475 | SEQ ID NO: 646 |
| Alternative intron chr9: 131085872-131087475 | SEQ ID NO: 647 |

LDAH

| | |
|---|---|
| Canonical exon chr2: 20686907-20687094 | SEQ ID NO: 648 |
| Alternative exon chr2: 20686838-20687094 | SEQ ID NO: 649 |
| Canonical intron chr2: 20685658-20686906 | SEQ ID NO: 650 |
| Alternative intron chr2: 20685658-20686837 | SEQ ID NO: 651 |

LIMS2

| | |
|---|---|
| Canonical exon chrcanonical_exon_chr2_127675014_127675102.txt | SEQ ID NO: 652 |
| Canonical exon chrcanonical_exon_chr2_127675014_127675190.txt | SEQ ID NO: 653 |
| Canonical exon chrcanonical_exon_chr2_127675014_127675286.txt | SEQ ID NO: 654 |
| Alternative exon chrnmd_exon_chr2_127674621_127675102.txt | SEQ ID NO: 655 |
| Alternative exon chrnmd_exon_chr2_127674621_127675190.txt | SEQ ID NO: 656 |
| Alternative exon chrnmd_exon_chr2_127674621_127675286.txt | SEQ ID NO: 657 |
| Canonical intron chrcanonical_intron_chr2_127673744_127675013.txt | SEQ ID NO: 658 |
| Alternative intron chrnmd_intron_chr2_127673744_127674620.txt | SEQ ID NO: 659 |

MALT1

| | |
|---|---|
| Canonical exon | SEQ ID NO: 660 |
| Canonical intron | SEQ ID NO: 661 |
| Alternative exon | SEQ ID NO: 662 |
| Alternative intron | SEQ ID NO: 663 |

MAP3K7

| | |
|---|---|
| Canonical exon chr6: 90560076-90560214 | SEQ ID NO: 664 |
| Alternative exon chr6: 90560076-90560234 | SEQ ID NO: 665 |
| Canonical intron chr6: 90560215-90561621 | SEQ ID NO: 666 |
| Alternative intron chr6: 90560235-90561621 | SEQ ID NO: 667 |

MAPK13

| | |
|---|---|
| Canonical exon chr6: 36130484-36130701 | SEQ ID NO: 668 |
| Canonical exon chr6: 36130513-36130701 | SEQ ID NO: 669 |
| Canonical exon chr6: 36130663-36130701 | SEQ ID NO: 670 |
| Alternative exon chr6: 36130484-36131157 | SEQ ID NO: 671 |
| Alternative exon chr6: 36130513-36131157 | SEQ ID NO: 672 |
| Alternative exon chr6: 36130663-36131157 | SEQ ID NO: 673 |
| Canonical intron chr6: 36130702-36131270 | SEQ ID NO: 674 |
| Alternative intron chr6: 36131158-36131270 | SEQ ID NO: 675 |

MCAT

| | |
|---|---|
| Canonical exon chr22: 43142926-43142964 | SEQ ID NO: 676 |
| Canonical exon chr22: 43142926-43143364 | SEQ ID NO: 677 |
| Canonical exon chr22: 43142926-43143398 | SEQ ID NO: 678 |
| Alternative exon chr22: 43142892-43142964 | SEQ ID NO: 679 |
| Alternative exon chr22: 43142892-43143364 | SEQ ID NO: 680 |
| Alternative exon chr22: 43142892-43143398 | SEQ ID NO: 681 |
| Canonical intron chr22: 43141250-43142925 | SEQ ID NO: 682 |
| Alternative intron chr22: 43141250-43142891 | SEQ ID NO: 683 |

MCEE

| | |
|---|---|
| Canonical exon chr2: 71130180-71130229 | SEQ ID NO: 684 |
| Canonical exon chr2: 71130180-71130239 | SEQ ID NO: 685 |
| Alternative exon chr2: 71129717-71130229 | SEQ ID NO: 686 |
| Alternative exon chr2: 71129717-71130239 | SEQ ID NO: 687 |
| Canonical intron chr2: 71124544-71130179 | SEQ ID NO: 688 |
| Alternative intron chr2: 71124544-71129716 | SEQ ID NO: 689 |

MPI

| | |
|---|---|
| Canonical exon chr15: 74890005-74890089 | SEQ ID NO: 690 |
| Canonical exon chr15: 74890039-74890089 | SEQ ID NO: 691 |
| Canonical exon chr15: 74890042-74890089 | SEQ ID NO: 692 |
| Canonical exon chr15: 74890052-74890089 | SEQ ID NO: 693 |
| Canonical exon chr15: 74890053-74890089 | SEQ ID NO: 694 |
| Canonical exon chr15: 74890062-74890089 | SEQ ID NO: 695 |
| Alternative exon chr15: 74890005-74890201 | SEQ ID NO: 696 |
| Alternative exon chr15: 74890039-74890201 | SEQ ID NO: 697 |
| Alternative exon chr15: 74890042-74890201 | SEQ ID NO: 698 |
| Alternative exon chr15: 74890052-74890201 | SEQ ID NO: 699 |
| Alternative exon chr15: 74890053-74890201 | SEQ ID NO: 700 |
| Alternative exon chr15: 74890062-74890201 | SEQ ID NO: 701 |
| Canonical intron chr15: 74890090-74890526 | SEQ ID NO: 702 |
| Alternative intron chr15: 74890202-74890526 | SEQ ID NO: 703 |

MSTO1

| | |
|---|---|
| Canonical exon chr1: 155611216-155611291 | SEQ ID NO: 704 |
| Alternative exon chr1: 155611216-155611355 | SEQ ID NO: 705 |
| Canonical intron chr1: 155611292-155611548 | SEQ ID NO: 706 |
| Alternative intron chr1: 155611356-155611548 | SEQ ID NO: 707 |

NBEAL2

| | |
|---|---|
| Canonical exon | SEQ ID NO: 708 |
| Canonical intron | SEQ ID NO: 709 |
| Alternative exon | SEQ ID NO: 710 |
| Alternative intron | SEQ ID NO: 711 |

NLE1

| | |
|---|---|
| Canonical exon | SEQ ID NO: 712 |
| Canonical intron | SEQ ID NO: 713 |
| Alternative exon | SEQ ID NO: 714 |
| Alternative intron | SEQ ID NO: 715 |

NLRC5

| | |
|---|---|
| Canonical exon | SEQ ID NO: 716 |
| Canonical intron | SEQ ID NO: 717 |
| Alternative exon | SEQ ID NO: 718 |
| Alternative intron | SEQ ID NO: 719 |

NOM1

| | |
|---|---|
| Canonical exon chr7: 156959851-156960174 | SEQ ID NO: 720 |
| Alternative exon chr7: 156959851-156960253 | SEQ ID NO: 721 |
| Canonical intron chr7: 156960175-156962150 | SEQ ID NO: 722 |
| Alternative intron chr7: 156960254-156962150 | SEQ ID NO: 723 |

NOP58

| | |
|---|---|
| Canonical exon chr2: 202291125-202291270 | SEQ ID NO: 724 |
| Alternative exon chr2: 202291021-202291270 | SEQ ID NO: 725 |
| Canonical intron chr2: 202290458-202291124 | SEQ ID NO: 726 |
| Alternative intron chr2: 202290458-202291020 | SEQ ID NO: 727 |

NPHP1

| | |
|---|---|
| Canonical exon chr2: 110178041-110178547 | SEQ ID NO: 728 |
| Canonical exon chr2: 110178423-110178547 | SEQ ID NO: 729 |
| Alternative exon chr2: 110178041-110178935 | SEQ ID NO: 730 |
| Alternative exon chr2: 110178423-110178935 | SEQ ID NO: 731 |
| Canonical intron chr2: 110178548-110179623 | SEQ ID NO: 732 |
| Alternative intron chr2: 110178936-110179623 | SEQ ID NO: 733 |

NPR1

| | |
|---|---|
| Canonical exon chr1: 153681704-153681839 | SEQ ID NO: 734 |
| Alternative exon chr1: 153681704-153682197 | SEQ ID NO: 735 |
| Canonical intron chr1: 153681840-153682497 | SEQ ID NO: 736 |
| Alternative intron chr1: 153682198-153682497 | SEQ ID NO: 737 |

NUP188

| | |
|---|---|
| Canonical exon chr9: 128988047-128988186 | SEQ ID NO: 738 |
| Alternative exon chr9: 128987916-128988186 | SEQ ID NO: 739 |
| Canonical intron chr9: 128987718-128988046 | SEQ ID NO: 740 |
| Alternative intron chr9: 128987718-128987915 | SEQ ID NO: 741 |

OSGEP

| | |
|---|---|
| Canonical exon | SEQ ID NO: 742 |
| Canonical intron | SEQ ID NO: 743 |
| Alternative exon | SEQ ID NO: 744 |
| Alternative intron | SEQ ID NO: 745 |

PABPC4

| | |
|---|---|
| Canonical exon chr1: 39571234-39571349 | SEQ ID NO: 746 |
| Alternative exon chr1: 39571097-39571349 | SEQ ID NO: 747 |
| Canonical intron chr1: 39570003-39571233 | SEQ ID NO: 748 |
| Alternative intron chr1: 39570003-39571096 | SEQ ID NO: 749 |

PCOLCE

| | |
|---|---|
| Canonical exon chr7: 100603959-100604217 | SEQ ID NO: 750 |
| Alternative exon chr7: 100603959-100604502 | SEQ ID NO: 751 |
| Canonical intron chr7: 100604218-100605090 | SEQ ID NO: 752 |
| Alternative intron chr7: 100604503-100605090 | SEQ ID NO: 753 |

PHKA2

| | |
|---|---|
| Canonical exon chrX: 18936055-18936150 | SEQ ID NO: 754 |
| Alternative exon chrX: 18936055-18936201 | SEQ ID NO: 755 |
| Canonical intron chrX: 18936151-18938626 | SEQ ID NO: 756 |
| Alternative intron chrX: 18936202-18938626 | SEQ ID NO: 757 |

PIDD1

| | |
|---|---|
| Canonical exon chr11: 802682-802891 | SEQ ID NO: 758 |
| Canonical exon chr11: 803174-803587 | SEQ ID NO: 759 |
| Alternative exon chr11: 802682-803085 | SEQ ID NO: 760 |
| Alternative exon chr11: 803174-803647 | SEQ ID NO: 761 |
| Canonical intron chr11: 802892-803173 | SEQ ID NO: 762 |
| Canonical intron chr11: 803588-804093 | SEQ ID NO: 763 |
| Alternative intron chr11: 803086-803173 | SEQ ID NO: 764 |
| Alternative intron chr11: 803648-804093 | SEQ ID NO: 765 |

PKD1

| | |
|---|---|
| Canonical exon chr16: 2105322-2105474 | SEQ ID NO: 766 |
| Alternative exon chr16: 2105322-2105662 | SEQ ID NO: 767 |
| Canonical intron chr16: 2105475-2105864 | SEQ ID NO: 768 |
| Alternative intron chr16: 2105663-2105864 | SEQ ID NO: 769 |

PLA2G6

| | |
|---|---|
| Canonical exon chr22: 38115527-38115681 | SEQ ID NO: 770 |
| Canonical exon chr22: 38115548-38115681 | SEQ ID NO: 771 |
| Alternative exon chr22: 38115527-38115849 | SEQ ID NO: 772 |
| Alternative exon chr22: 38115548-38115849 | SEQ ID NO: 773 |
| Canonical intron chr22: 38115682-38116074 | SEQ ID NO: 774 |
| Alternative intron chr22: 38115850-38116074 | SEQ ID NO: 775 |

PLD2

| | |
|---|---|
| Canonical exon chr17: 4814418-4814501 | SEQ ID NO: 776 |
| Canonical exon chr17: 4816620-4816746 | SEQ ID NO: 777 |
| Canonical exon chr17: 4818297-4818385 | SEQ ID NO: 778 |
| Alternative exon chr17: 4814367-4814501 | SEQ ID NO: 779 |
| Alternative exon chr17: 4816542-4816746 | SEQ ID NO: 780 |
| Alternative exon chr17: 4818297-4818406 | SEQ ID NO: 781 |
| Canonical intron chr17: 4810952-4814417 | SEQ ID NO: 782 |
| Canonical intron chr17: 4815935-4816619 | SEQ ID NO: 783 |
| Canonical intron chr17: 4818386-4818493 | SEQ ID NO: 784 |
| Alternative intron chr17: 4810952-4814366 | SEQ ID NO: 785 |
| Alternative intron chr17: 4815935-4816541 | SEQ ID NO: 786 |
| Alternative intron chr17: 4818407-4818493 | SEQ ID NO: 787 |

PLEKHG5

| | |
|---|---|
| Canonical exon chr1: 6470990-6471100 | SEQ ID NO: 788 |
| Alternative exon chr1: 6470990-6471240 | SEQ ID NO: 789 |
| Canonical intron chr1: 6471101-6471487 | SEQ ID NO: 790 |
| Alternative intron chr1: 6471241-6471487 | SEQ ID NO: 791 |

PNPO

| | |
|---|---|
| Canonical exon | SEQ ID NO: 792 |
| Canonical exon | SEQ ID NO: 793 |
| Canonical exon | SEQ ID NO: 794 |
| Canonical exon | SEQ ID NO: 795 |
| Canonical exon | SEQ ID NO: 796 |
| Canonical intron | SEQ ID NO: 797 |
| Alternative exon | SEQ ID NO: 798 |
| Alternative exon | SEQ ID NO: 799 |
| Alternative exon | SEQ ID NO: 800 |
| Alternative exon | SEQ ID NO: 801 |
| Alternative exon | SEQ ID NO: 802 |
| Alternative intron | SEQ ID NO: 803 |

POLE

| | |
|---|---|
| Canonical exon | SEQ ID NO: 804 |
| Canonical intron | SEQ ID NO: 805 |
| Alternative exon | SEQ ID NO: 806 |
| Alternative intron | SEQ ID NO: 807 |

PON2

| | |
|---|---|
| Canonical exon chr7: 95416242-95416297 | SEQ ID NO: 808 |
| Alternative exon chr7: 95416172-95416297 | SEQ ID NO: 809 |
| Canonical intron chr7: 95412478-95416241 | SEQ ID NO: 810 |
| Alternative intron chr7: 95412478-95416171 | SEQ ID NO: 811 |

PRMT7

| | |
|---|---|
| Canonical exon chr16: 68353492-68353566 | SEQ ID NO: 812 |
| Alternative exon chr16: 68353395-68353566 | SEQ ID NO: 813 |
| Canonical intron chr16: 68352410-68353491 | SEQ ID NO: 814 |
| Alternative intron chr16: 68352410-68353394 | SEQ ID NO: 815 |

PRODH

| | |
|---|---|
| Canonical exon | SEQ ID NO: 816 |
| Canonical intron | SEQ ID NO: 817 |
| Alternative exon | SEQ ID NO: 818 |
| Alternative intron | SEQ ID NO: 819 |

PRPF3

| | |
|---|---|
| Canonical exon | SEQ ID NO: 820 |
| Canonical intron | SEQ ID NO: 821 |
| Alternative exon | SEQ ID NO: 822 |
| Alternative intron | SEQ ID NO: 823 |

PRPF4

| | |
|---|---|
| Canonical exon | SEQ ID NO: 824 |
| Canonical intron | SEQ ID NO: 825 |
| Alternative exon | SEQ ID NO: 826 |
| Alternative intron | SEQ ID NO: 827 |

PYCR1

| | |
|---|---|
| Canonical exon chr17: 81937180-81937239 | SEQ ID NO: 828 |
| Alternative exon chr17: 81936970-81937239 | SEQ ID NO: 829 |
| Canonical intron chr17: 81936862-81937179 | SEQ ID NO: 830 |
| Alternative intron chr17: 81936862-81936969 | SEQ ID NO: 831 |

RAD52

| | |
|---|---|
| Canonical exon | SEQ ID NO: 832 |
| Canonical exon | SEQ ID NO: 833 |
| Canonical intron | SEQ ID NO: 834 |
| Canonical intron | SEQ ID NO: 835 |
| Alternative exon | SEQ ID NO: 836 |
| Alternative exon | SEQ ID NO: 837 |
| Alternative intron | SEQ ID NO: 838 |
| Alternative intron | SEQ ID NO: 839 |

REXO1

| | |
|---|---|
| Canonical exon chr19: 1817219-1817329 | SEQ ID NO: 840 |
| Canonical exon chr19: 1819934-1820057 | SEQ ID NO: 841 |
| Alternative exon chr19: 1817219-1817458 | SEQ ID NO: 842 |
| Alternative exon chr19: 1819934-1820180 | SEQ ID NO: 843 |
| Canonical intron chr19: 1817330-1817706 | SEQ ID NO: 844 |
| Canonical intron chr19: 1820058-1820263 | SEQ ID NO: 845 |
| Alternative intron chr19: 1817459-1817706 | SEQ ID NO: 846 |
| Alternative intron chr19: 1820181-1820263 | SEQ ID NO: 847 |

RFX5

| | |
|---|---|
| Canonical exon chr1: 151344417-151344536 | SEQ ID NO: 848 |
| Canonical exon chr1: 151344521-151344536 | SEQ ID NO: 849 |
| Alternative exon chr1: 151344417-151344604 | SEQ ID NO: 850 |
| Alternative exon chr1: 151344521-151344604 | SEQ ID NO: 851 |
| Canonical intron chr1: 151344537-151344727 | SEQ ID NO: 852 |
| Alternative intron chr1: 151344605-151344727 | SEQ ID NO: 853 |

RHBDF2

| | |
|---|---|
| Canonical exon chr17: 76474730-76474804 | SEQ ID NO: 854 |
| Alternative exon chr17: 76474660-76474804 | SEQ ID NO: 855 |
| Canonical intron chr17: 76474535-76474729 | SEQ ID NO: 856 |
| Alternative intron chr17: 76474535-76474659 | SEQ ID NO: 857 |

RMND1

| | |
|---|---|
| Canonical exon chr6: 151436446-151436554 | SEQ ID NO: 858 |
| Alternative exon chr6: 151436420-151436554 | SEQ ID NO: 859 |
| Canonical intron chr6: 151433231-151436445 | SEQ ID NO: 860 |
| Alternative intron chr6: 151433231-151436419 | SEQ ID NO: 861 |

ROBO3

| | |
|---|---|
| Canonical exon | SEQ ID NO: 862 |
| Canonical exon | SEQ ID NO: 863 |
| Canonical exon | SEQ ID NO: 864 |
| Canonical intron | SEQ ID NO: 865 |
| Canonical intron | SEQ ID NO: 866 |
| Alternative exon | SEQ ID NO: 867 |
| Alternative exon | SEQ ID NO: 868 |
| Alternative exon | SEQ ID NO: 869 |
| Alternative intron | SEQ ID NO: 870 |
| Alternative intron | SEQ ID NO: 871 |

RPGRIP1

| | |
|---|---|
| Canonical exon chr14: 21321259-21321402 | SEQ ID NO: 872 |
| Canonical exon chr14: 21321364-21321402 | SEQ ID NO: 873 |
| Canonical exon chr14: 21321373-21321402 | SEQ ID NO: 874 |
| Alternative exon chr14: 21321259-21321629 | SEQ ID NO: 875 |
| Alternative exon chr14: 21321364-21321629 | SEQ ID NO: 876 |
| Alternative exon chr14: 21321373-21321629 | SEQ ID NO: 877 |
| Canonical intron chr14: 21321403-21321853 | SEQ ID NO: 878 |
| Alternative intron chr14: 21321630-21321853 | SEQ ID NO: 879 |

RTTN

| | |
|---|---|
| Canonical exon | SEQ ID NO: 880 |
| Canonical intron | SEQ ID NO: 881 |
| Alternative exon | SEQ ID NO: 882 |
| Alternative intron | SEQ ID NO: 883 |

RUFY3

| | |
|---|---|
| Canonical exon chr4: 70793785-70793904 | SEQ ID NO: 884 |
| Alternative exon chr4: 70793765-70793904 | SEQ ID NO: 885 |
| Canonical intron chr4: 70789593-70793784 | SEQ ID NO: 886 |
| Alternative intron chr4: 70789593-70793764 | SEQ ID NO: 887 |

SEMA3B

| | |
|---|---|
| Canonical exon chr3: 50275302-50275459 | SEQ ID NO: 888 |
| Alternative exon chr3: 50275177-50275459 | SEQ ID NO: 889 |
| Canonical intron chr3: 50275054-50275301 | SEQ ID NO: 890 |
| Alternative intron chr3: 50275054-50275176 | SEQ ID NO: 891 |

SEMA3F

| | |
|---|---|
| Canonical exon chr3: 50185443-50185531 | SEQ ID NO: 892 |
| Alternative exon chr3: 50185403-50185531 | SEQ ID NO: 893 |
| Canonical intron chr3: 50184815-50185442 | SEQ ID NO: 894 |
| Alternative intron chr3: 50184815-50185402 | SEQ ID NO: 895 |

SH2D3A

| | |
|---|---|
| Canonical exon chr19: 6754052-6754425 | SEQ ID NO: 896 |
| Canonical exon chr19: 6754251-6754425 | SEQ ID NO: 897 |
| Alternative exon chr19: 6754052-6754448 | SEQ ID NO: 898 |
| Alternative exon chr19: 6754251-6754448 | SEQ ID NO: 899 |
| Canonical intron chr19: 6754426-6754615 | SEQ ID NO: 900 |
| Alternative intron chr19: 6754449-6754615 | SEQ ID NO: 901 |

SIK3

| | |
|---|---|
| Canonical exon | SEQ ID NO: 902 |
| Canonical intron | SEQ ID NO: 903 |
| Alternative exon | SEQ ID NO: 904 |
| Alternative intron | SEQ ID NO: 905 |

SIRT3

| | |
|---|---|
| Canonical exon chr11: 230452-230552 | SEQ ID NO: 906 |
| Alternative exon chr11: 230452-230706 | SEQ ID NO: 907 |
| Canonical intron chr11: 230553-232982 | SEQ ID NO: 908 |
| Alternative intron chr11: 230707-232982 | SEQ ID NO: 909 |

SKIV2L

| | |
|---|---|
| Canonical exon chr6: 31968686-31968796 | SEQ ID NO: 910 |
| Alternative exon chr6: 31968623-31968796 | SEQ ID NO: 911 |
| Canonical intron chr6: 31968539-31968685 | SEQ ID NO: 912 |
| Alternative intron chr6: 31968539-31968622 | SEQ ID NO: 913 |

SLC2A13

| | |
|---|---|
| Canonical exon chr12: 39830103-39830228 | SEQ ID NO: 914 |
| Alternative exon chr12: 39830103-39830480 | SEQ ID NO: 915 |
| Canonical intron chr12: 39830229-39864761 | SEQ ID NO: 916 |
| Alternative intron chr12: 39830481-39864761 | SEQ ID NO: 917 |

SLC12A7

| | |
|---|---|
| Canonical exon chr5: 1076138-1076236 | SEQ ID NO: 918 |
| Canonical intron chr5: 1075491-1076137 | SEQ ID NO: 919 |
| Alternative exon chr5: 1075891-1076236 | SEQ ID NO: 920 |
| Alternative intron chr5: 1075491-1075890 | SEQ ID NO: 921 |

SLC22A3

| | |
|---|---|
| Canonical exon chr6: 160348378-160348848 | SEQ ID NO: 922 |
| Alternative exon chr6: 160348378-160349058 | SEQ ID NO: 923 |
| Canonical intron chr6: 160348849-160397978 | SEQ ID NO: 924 |
| Alternative intron chr6: 160349059-160397978 | SEQ ID NO: 925 |

SLC25A13

| | |
|---|---|
| Canonical exon chr7: 96170045-96170125 | SEQ ID NO: 926 |
| Alternative exon chr7: 96170045-96170149 | SEQ ID NO: 927 |
| Canonical intron chr7: 96170126-96171471 | SEQ ID NO: 928 |
| Alternative intron chr7: 96170150-96171471 | SEQ ID NO: 929 |

SLC25A37

| | |
|---|---|
| Canonical exon chr8: 23566108-23566336 | SEQ ID NO: 930 |
| Alternative exon chr8: 23566108-23566813 | SEQ ID NO: 931 |
| Canonical intron chr8: 23566337-23568321 | SEQ ID NO: 932 |
| Alternative intron chr8: 23566814-s23568321 | SEQ ID NO: 933 |

SLC27A5

| | |
|---|---|
| Canonical intron | SEQ ID NO: 934 |
| Canonical exon | SEQ ID NO: 935 |
| Alternative intron | SEQ ID NO: 936 |
| Alternative exon | SEQ ID NO: 937 |

SLC30A9

| | |
|---|---|
| Canonical exon chr4: 42066550-42066621 | SEQ ID NO: 938 |
| Alternative exon chr4: 42066485-42066621 | SEQ ID NO: 939 |
| Canonical intron chr4: 42065350-42066549 | SEQ ID NO: 940 |
| Alternative intron chr4: 42065350-42066484 | SEQ ID NO: 941 |

SLC30A10

| | |
|---|---|
| Canonical exon chr1: 219927028-219927105 | SEQ ID NO: 942 |
| Alternative exon chr1: 219927028-219927143 | SEQ ID NO: 943 |
| Canonical intron chr1: 219927106-219927800 | SEQ ID NO: 944 |
| Alternative intron chr1: 219927144-219927800 | SEQ ID NO: 945 |

SMPD1

| | |
|---|---|
| Canonical exon chr11: 6391384-6392156 | SEQ ID NO: 946 |
| Canonical exon chr11: 6391387-6392156 | SEQ ID NO: 947 |
| Alternative exon chr11: 6391384-6392196 | SEQ ID NO: 948 |
| Alternative exon chr11: 6391387-6392196 | SEQ ID NO: 949 |
| Canonical intron chr11: 6392157-6393215 | SEQ ID NO: 950 |
| Alternative intron chr11: 6392197-6393215 | SEQ ID NO: 951 |

SMTN

| | |
|---|---|
| Canonical exon chr22: 31088513-31088574 | SEQ ID NO: 952 |
| Canonical exon chr22: 31088513-31088605 | SEQ ID NO: 953 |
| Canonical exon chr22: 31088513-31088606 | SEQ ID NO: 954 |
| Alternative exon chr22: 31088454-31088574 | SEQ ID NO: 955 |
| Alternative exon chr22: 31088454-31088605 | SEQ ID NO: 956 |
| Alternative exon chr22: 31088454-31088606 | SEQ ID NO: 957 |
| Canonical intron chr22: 31088114-31088512 | SEQ ID NO: 958 |
| Alternative intron chr22: 31088114-31088453 | SEQ ID NO: 959 |

SNRPB

| | |
|---|---|
| Canonical exon chr20: 2467607-2467758 | SEQ ID NO: 960 |
| Alternative exon chr20: 2467192-2467758 | SEQ ID NO: 961 |
| Canonical intron chr20: 2465820-2467606 | SEQ ID NO: 962 |
| Alternative intron chr20: 2465820-2467191 | SEQ ID NO: 963 |

SP140

| | |
|---|---|
| Canonical exon chr2: 230309924-230310039 | SEQ ID NO: 964 |
| Alternative exon chr2: 230309924-230310211 | SEQ ID NO: 965 |
| Canonical intron chr2: 230310040-230310742 | SEQ ID NO: 966 |
| Alternative intron chr2: 230310212-230310742 | SEQ ID NO: 967 |

STAMBP

| | |
|---|---|
| Canonical exon chr2: 73844813-73844888 | SEQ ID NO: 968 |
| Alternative exon chr2: 73844813-73845036 | SEQ ID NO: 969 |
| Canonical intron chr2: 73844889-73845166 | SEQ ID NO: 970 |
| Alternative intron chr2: 73845037-73845166 | SEQ ID NO: 971 |

STXBP2

| | |
|---|---|
| Canonical exon chr19: 7646249-7646344 | SEQ ID NO: 972 |
| Alternative exon chr19: 7645760-7646344 | SEQ ID NO: 973 |
| Canonical intron chr19: 7645307-7646248 | SEQ ID NO: 974 |
| Alternative intron chr19: 7645307-7645759 | SEQ ID NO: 975 |

SULF2

| | |
|---|---|
| Canonical exon chr20: 47666260-47666488 | SEQ ID NO: 976 |
| Alternative exon chr20: 47666088-47666488 | SEQ ID NO: 977 |
| Canonical intron chr20: 47665954-47666259 | SEQ ID NO: 978 |
| Alternative intron chr20: 47665954-47666087 | SEQ ID NO: 979 |

SYNGAP1

| | |
|---|---|
| Canonical exon | SEQ ID NO: 980 |
| Canonical intron | SEQ ID NO: 981 |
| Alternative exon | SEQ ID NO: 982 |
| Alternative intron | SEQ ID NO: 983 |

SYNJ2

| | |
|---|---|
| Canonical exon chr6: 158061992-158062164 | SEQ ID NO: 984 |
| Alternative exon chr6: 158061992-158062221 | SEQ ID NO: 985 |
| Canonical intron chr6: 158062165-158063790 | SEQ ID NO: 986 |
| Alternative intron chr6: 158062222-158063790 | SEQ ID NO: 987 |

TAZ

| | |
|---|---|
| Canonical exon | SEQ ID NO: 988 |
| Canonical intron | SEQ ID NO: 989 |
| Alternative exon | SEQ ID NO: 990 |
| Alternative intron | SEQ ID NO: 991 |

TBL2

| | |
|---|---|
| Canonical exon chr7: 73574383-73574513 | SEQ ID NO: 992 |
| Alternative exon chr7: 73574383-73574804 | SEQ ID NO: 993 |
| Canonical intron chr7: 73574514-73578419 | SEQ ID NO: 994 |
| Alternative intron chr7: 73574805-73578419 | SEQ ID NO: 995 |

TDRD7

| | |
|---|---|
| Canonical exon chr9: 97478439-97478573 | SEQ ID NO: 996 |
| Alternative exon chr9: 97478439-97478635 | SEQ ID NO: 997 |
| Canonical intron chr9: 97478574-97480827 | SEQ ID NO: 998 |
| Alternative intron chr9: 97478636-97480827 | SEQ ID NO: 999 |

TECPR2

| | |
|---|---|
| Canonical exon chr14: 102497570-102497719 | SEQ ID NO: 1000 |
| Alternative exon chr14: 102497469-102497719 | SEQ ID NO: 1001 |
| Canonical intron chr14: 102497121-102497569 | SEQ ID NO: 1002 |
| Alternative intron chr14: 102497121-102497468 | SEQ ID NO: 1003 |

TFB1M

| | |
|---|---|
| Canonical exon chr6: 155314296-155314484 | SEQ ID NO: 1004 |
| Alternative exon chr6: 155314117-155314484 | SEQ ID NO: 1005 |
| Canonical intron chr6: 155311340-155314295 | SEQ ID NO: 1006 |
| Alternative intron chr6: 155311340-155314116 | SEQ ID NO: 1007 |

TM6SF1

| | |
|---|---|
| Canonical exon chr15: 83119578-83119681 | SEQ ID NO: 1008 |
| Alternative exon chr15: 83119578-83119734 | SEQ ID NO: 1009 |
| Canonical intron chr15: 83119682-83121920 | SEQ ID NO: 1010 |
| Alternative intron chr15: 83119735-83121920 | SEQ ID NO: 1011 |

TNK2

| | |
|---|---|
| Canonical exon chr3: 195870114-195870205 | SEQ ID NO: 1012 |
| Alternative exon chr3: 195870114-195870378 | SEQ ID NO: 1013 |
| Canonical intron chr3: 195870206-195872275 | SEQ ID NO: 1014 |
| Alternative intron chr3: 195870379-195872275 | SEQ ID NO: 1015 |

TOE1

| | |
|---|---|
| Canonical exon chr1: 45342843-45343002 | SEQ ID NO: 1016 |
| Alternative exon chr1: 45342735-45343002 | SEQ ID NO: 1017 |
| Canonical intron chr1: 45342644-45342842 | SEQ ID NO: 1018 |
| Alternative intron chr1: 45342644-45342734 | SEQ ID NO: 1019 |

TOR1B

| | |
|---|---|
| Canonical exon | SEQ ID NO: 1020 |
| Canonical intron | SEQ ID NO: 1021 |
| Alternative exon | SEQ ID NO: 1022 |
| Alternative intron | SEQ ID NO: 1023 |

TRAPPC6B

| | |
|---|---|
| Canonical exon chr14: 39159483-39159550 | SEQ ID NO: 1024 |
| Alternative exon chr14: 39159450-39159550 | SEQ ID NO: 1025 |
| Canonical intron chr14: 39158403-39159482 | SEQ ID NO: 1026 |
| Alternative intron chr14: 39158403-39159449 | SEQ ID NO: 1027 |

TRIP4

| | |
|---|---|
| Canonical exon chr15: 64387836-64387964 | SEQ ID NO: 1028 |
| Alternative exon chr15: 64387836-64388048 | SEQ ID NO: 1029 |
| Canonical intron chr15: 64387965-64393945 | SEQ ID NO: 1030 |
| Alternative intron chr15: 64388049-64393945 | SEQ ID NO: 1031 |

TRMT2A

| | |
|---|---|
| Canonical exon | SEQ ID NO: 1032 |
| Canonical intron | SEQ ID NO: 1033 |
| Alternative exon | SEQ ID NO: 1034 |
| Alternative intron | SEQ ID NO: 1035 |

TRMU

| | |
|---|---|
| Canonical exon chr22: 46335714-46335846 | SEQ ID NO: 1036 |
| Canonical exon chr22: 46335725-46335846 | SEQ ID NO: 1037 |
| Canonical exon chr22: 46335745-46335846 | SEQ ID NO: 1038 |
| Alternative exon chr22: 46335714-46336106 | SEQ ID NO: 1039 |
| Alternative exon chr22: 46335725-46336106 | SEQ ID NO: 1040 |
| Alternative exon chr22: 46335745-46336106 | SEQ ID NO: 1041 |
| Canonical intron chr22: 46335847-46337778 | SEQ ID NO: 1042 |
| Alternative intron chr22: 46336107-46337778 | SEQ ID NO: 1043 |

TRPV4

| | |
|---|---|
| Canonical exon chr12: 109788400-109788716 | SEQ ID NO: 1044 |
| Alternative exon chr12: 109788400-109788742 | SEQ ID NO: 1045 |
| Canonical intron chr12: 109788717-109792362 | SEQ ID NO: 1046 |
| Alternative intron chr12: 109788743-109792362 | SEQ ID NO: 1047 |

TTC19

| | |
|---|---|
| Canonical exon chr17: 16000118-16000245 | SEQ ID NO: 1048 |
| Alternative exon chr17: 16000118-16000382 | SEQ ID NO: 1049 |
| Canonical intron chr17: 16000246-16001914 | SEQ ID NO: 1050 |
| Alternative intron chr17: 16000383-16001914 | SEQ ID NO: 1051 |

TYMP

| | |
|---|---|
| Canonical exon chr22: 50527165-50527283 | SEQ ID NO: 1052 |
| Canonical exon chr22: 50527165-50527328 | SEQ ID NO: 1053 |
| Alternative exon chr22: 50526898-50527283 | SEQ ID NO: 1054 |
| Alternative exon chr22: 50526898-50527328 | SEQ ID NO: 1055 |
| Canonical intron chr22: 50526739-50527164 | SEQ ID NO: 1056 |
| Alternative intron chr22: 50526739-50526897 | SEQ ID NO: 1057 |

UMPS

| | |
|---|---|
| Canonical exon chr3: 124737568-124738239 | SEQ ID NO: 1058 |
| Alternative exon chr3: 124737568-124738443 | SEQ ID NO: 1059 |
| Canonical intron chr3: 124738240-124740023 | SEQ ID NO: 1060 |
| Alternative intron chr3: 124738444-124740023 | SEQ ID NO: 1061 |

UROD

| | |
|---|---|
| Canonical exon chr1: 45012907-45013019 | SEQ ID NO: 1062 |
| Alternative exon chr1: 45012857-45013019 | SEQ ID NO: 1063 |
| Canonical intron chr1: 45012286-45012906 | SEQ ID NO: 1064 |
| Alternative intron chr1: 45012286-45012856 | SEQ ID NO: 1065 |

WDR11

| | |
|---|---|
| Canonical exon chr10: 120851362-120851506 | SEQ ID NO: 1066 |
| Alternative exon chr10: 120851362-120851527 | SEQ ID NO: 1067 |
| Canonical intron chr10: 120851507-120852523 | SEQ ID NO: 1068 |
| Alternative intron chr10: 120851528-120852523 | SEQ ID NO: 1069 |

WDR62

| | |
|---|---|
| Canonical exon | SEQ ID NO: 1070 |
| Canonical intron | SEQ ID NO: 1071 |
| Alternative exon | SEQ ID NO: 1072 |
| Alternative intron | SEQ ID NO: 1073 |

WIPI1

| | |
|---|---|
| Canonical exon chr17: 68434556-68434626 | SEQ ID NO: 1074 |
| Alternative exon chr17: 68434420-68434626 | SEQ ID NO: 1075 |
| Canonical intron chr17: 68433576-68434555 | SEQ ID NO: 1076 |
| Alternative intron chr17: 68433576-68434419 | SEQ ID NO: 1077 |

WRAP53

| | |
|---|---|
| Canonical exon chr17: 7701459-7701549 | SEQ ID NO: 1078 |
| Alternative exon chr17: 7701427-7701549 | SEQ ID NO: 1079 |
| Canonical intron chr17: 7700830-7701458 | SEQ ID NO: 1080 |
| Alternative intron chr17: 7700830-7701426 | SEQ ID NO: 1081 |

XPO1

| | |
|---|---|
| Canonical exon chr2: 61492035-61492198 | SEQ ID NO: 1082 |
| Alternative exon chr2: 61492035-61492250 | SEQ ID NO: 1083 |
| Canonical intron chr2: 61492199-61492324 | SEQ ID NO: 1084 |
| Alternative intron chr2: 61492251-61492324 | SEQ ID NO: 1085 |

YY1AP1

| | |
|---|---|
| Canonical exon chr1: 155688930-155689000 | SEQ ID NO: 1086 |
| Alternative exon chr1: 155688579-155689000 | SEQ ID NO: 1087 |
| Canonical intron chr1: 155688202-155688929 | SEQ ID NO: 1088 |
| Alternative intron chr1: 155688202-155688578 | SEQ ID NO: 1089 |

ZC3H14

| | |
|---|---|
| Canonical exon chr14: 88607243-88607363 | SEQ ID NO: 1090 |
| Canonical exon chr14: 88607284-88607363 | SEQ ID NO: 1091 |
| Canonical intron chr14: 88607364-88609266 | SEQ ID NO: 1092 |
| Alternative exon chr14: 88607243-88607394 | SEQ ID NO: 1093 |
| Alternative exon chr14: 88607243-88607425 | SEQ ID NO: 1094 |
| Alternative exon chr14: 88607284-88607394 | SEQ ID NO: 1095 |
| Alternative exon chr14: 88607284-88607425 | SEQ ID NO: 1096 |
| Alternative intron chr14: 88607395-88609266 | SEQ ID NO: 1097 |
| Alternative intron chr14: 88607426-88609266 | SEQ ID NO: 1098 |

TABLE 4
Target: ABCA5

| | |
|---|---|
| pre-mRNA | ENST00000392676.8 (SEQ ID NO: 15288); ENST00000586601.1 (SEQ ID NO: 10230); |
| transcript | ENST00000586811.1 (SEQ ID NO: 10232); ENST00000586995.5 (SEQ ID NO: 15289); |
| | ENST00000587607.5 (SEQ ID NO: 10235); ENST00000588106.1 (SEQ ID NO: 10237); |
| | ENST00000588665.5 (SEQ ID NO: 10239); ENST00000588877.5 (SEQ ID NO: 15290); |
| | ENST00000589609.1 (SEQ ID NO: 10242); ENST00000589975.5 (SEQ ID NO: 10244); |
| | ENST00000591234.5 (SEQ ID NO: 15291); ENST00000592568.1 (SEQ ID NO: 10247); |
| | ENST00000593153.5 (SEQ ID NO: 15292); ENST00000593253.5 (SEQ ID NO: 10250) |
| transcript | ENST00000392676.8 (SEQ ID NO: 10229); ENST00000586601.1 (SEQ ID NO: 10231); |
| | ENST00000586811.1 (SEQ ID NO: 10233); ENST00000586995.5 (SEQ ID NO: 10234); |
| | ENST00000587607.5 (SEQ ID NO: 10236); ENST00000588106.1 (SEQ ID NO: 10238); |
| | ENST00000588665.5 (SEQ ID NO: 10240); ENST00000588877.5 (SEQ ID NO: 10241); |
| | ENST00000589609.1 (SEQ ID NO: 10243); ENST00000589975.5 (SEQ ID NO: 10245); |
| | ENST00000591234.5 (SEQ ID NO: 10246); ENST00000592568.1 (SEQ ID NO: 10248); |
| | ENST00000593153.5 (SEQ ID NO: 10249); ENST00000593253.5 (SEQ ID NO: 10251) |

Target: ABCA7

| | |
|---|---|
| pre-mRNA | ENST00000263094.11 (SEQ ID NO: 10252); ENST00000433129.6 (SEQ ID NO: 10254); |
| transcript | ENST00000435683.7 (SEQ ID NO: 10256); ENST00000524383.1 (SEQ ID NO: 10258); |
| | ENST00000524850.5 (SEQ ID NO: 10260); ENST00000525073.6 (SEQ ID NO: 10262); |
| | ENST00000525238.2 (SEQ ID NO: 10264); ENST00000526885.5 (SEQ ID NO: 10266); |
| | ENST00000527496.1 (SEQ ID NO: 10268); ENST00000529442.7 (SEQ ID NO: 10270); |
| | ENST00000530092.2 (SEQ ID NO: 10272); ENST00000530703.1 (SEQ ID NO: 10274); |
| | ENST00000531467.5 (SEQ ID NO: 10276); ENST00000531478.5 (SEQ ID NO: 10278); |
| | ENST00000532194.3 (SEQ ID NO: 10280); ENST00000533574.1 (SEQ ID NO: 10282); |
| | ENST00000612569.1 (SEQ ID NO: 10284); ENST00000673773.1 (SEQ ID NO: 10286) |
| transcript | ENST00000263094.11 (SEQ ID NO: 10253); ENST00000433129.6 (SEQ ID NO: 10255); |
| | ENST00000435683.7 (SEQ ID NO: 10257); ENST00000524383.1 (SEQ ID NO: 10259); |
| | ENST00000524850.5 (SEQ ID NO: 10261); ENST00000525073.6 (SEQ ID NO: 10263); |
| | ENST00000525238.2 (SEQ ID NO: 10265); ENST00000526885.5 (SEQ ID NO: 10267); |
| | ENST00000527496.1 (SEQ ID NO: 10269); ENST00000529442.7 (SEQ ID NO: 10271); |
| | ENST00000530092.2 (SEQ ID NO: 10273); ENST00000530703.1 (SEQ ID NO: 10275); |
| | ENST00000531467.5 (SEQ ID NO: 10277); ENST00000531478.5 (SEQ ID NO: 10279); |
| | ENST00000532194.3 (SEQ ID NO: 10281); ENST00000533574.1 (SEQ ID NO: 10283); |
| | ENST00000612569.1 (SEQ ID NO: 10285); ENST00000673773.1 (SEQ ID NO: 10287) |

Target: ABCC3

| | |
|---|---|
| pre-mRNA | ENST00000285238.13 (SEQ ID NO: 15293); ENST00000427699.5 (SEQ ID NO: 15294); |
| transcript | ENST00000502426.5 (SEQ ID NO: 15295); ENST00000503304.1 (SEQ ID NO: 10291); |
| | ENST00000503337.1 (SEQ ID NO: 10293); ENST00000504586.1 (SEQ ID NO: 10295); |
| | ENST00000505699.5 (SEQ ID NO: 15296); ENST00000506464.5 (SEQ ID NO: 10298); |
| | ENST00000508929.1 (SEQ ID NO: 10300); ENST00000510633.5 (SEQ ID NO: 10302); |
| | ENST00000510891.1 (SEQ ID NO: 10304); ENST00000513511.5 (SEQ ID NO: 10306); |
| | ENST00000513589.1 (SEQ ID NO: 10308); ENST00000513745.1 (SEQ ID NO: 10310); |
| | ENST00000515070.1 (SEQ ID NO: 10312); ENST00000515585.1 (SEQ ID NO: 10314); |
| | ENST00000515707.1 (SEQ ID NO: 10316); ENST00000571855.1 (SEQ ID NO: 10318) |
| transcript | ENST00000285238.13 (SEQ ID NO: 10288); ENST00000427699.5 (SEQ ID NO: 10289); |
| | ENST00000502426.5 (SEQ ID NO: 10290); ENST00000503304.1 (SEQ ID NO: 10292); |
| | ENST00000503337.1 (SEQ ID NO: 10294); ENST00000504586.1 (SEQ ID NO: 10296); |
| | ENST00000505699.5 (SEQ ID NO: 10297); ENST00000506464.5 (SEQ ID NO: 10299); |
| | ENST00000508929.1 (SEQ ID NO: 10301); ENST00000510633.5 (SEQ ID NO: 10303); |
| | ENST00000510891.1 (SEQ ID NO: 10305); ENST00000513511.5 (SEQ ID NO: 10307); |
| | ENST00000513589.1 (SEQ ID NO: 10309); ENST00000513745.1 (SEQ ID NO: 10311); |
| | ENST00000515070.1 (SEQ ID NO: 10313); ENST00000515585.1 (SEQ ID NO: 10315); |
| | ENST00000515707.1 (SEQ ID NO: 10317); ENST00000571855.1 (SEQ ID NO: 10319) |

Target: ABCC5

| | |
|---|---|
| pre-mRNA | ENST00000265586.10 (SEQ ID NO: 15297); ENST00000334444.11 (SEQ ID NO: 15298); |
| transcript | ENST00000382494.6 (SEQ ID NO: 15299); ENST00000392579.6 (SEQ ID NO: 15300); |
| | ENST00000427120.6 (SEQ ID NO: 15301); ENST00000437205.5 (SEQ ID NO: 15302); |
| | ENST00000437341.5 (SEQ ID NO: 10326); ENST00000438979.6 (SEQ ID NO: 15303); |
| | ENST00000443376.5 (SEQ ID NO: 15304); ENST00000443497.1 (SEQ ID NO: 10330); |
| | ENST00000446941.2 (SEQ ID NO: 10332); ENST00000476402.1 (SEQ ID NO: 10334); |
| | ENST00000492216.1 (SEQ ID NO: 10336) |
| transcript | ENST00000265586.10 (SEQ ID NO: 10320); ENST00000334444.11 (SEQ ID NO: 10321); |
| | ENST00000382494.6 (SEQ ID NO: 10322); ENST00000392579.6 (SEQ ID NO: 10323); |
| | ENST00000427120.6 (SEQ ID NO: 10324); ENST00000437205.5 (SEQ ID NO: 10325); |
| | ENST00000437341.5 (SEQ ID NO: 10327); ENST00000438979.6 (SEQ ID NO: 10328); |
| | ENST00000443376.5 (SEQ ID NO: 10329); ENST00000443497.1 (SEQ ID NO: 10331); |
| | ENST00000446941.2 (SEQ ID NO: 10333); ENST00000476402.1 (SEQ ID NO: 10335); |
| | ENST00000492216.1 (SEQ ID NO: 10337) |

Target: ABCC8

| | |
|---|---|
| pre-mRNA | ENST00000302539.9 (SEQ ID NO: 15305); ENST00000389817.7 (SEQ ID NO: 15306); |
| transcript | ENST00000524561.1 (SEQ ID NO: 10340); ENST00000525022.1 (SEQ ID NO: 10342); |
| | ENST00000526002.1 (SEQ ID NO: 10344); ENST00000526037.5 (SEQ ID NO: 10346); |
| | ENST00000526168.5 (SEQ ID NO: 10348); ENST00000526921.5 (SEQ ID NO: 10350); |
| | ENST00000527905.5 (SEQ ID NO: 15307); ENST00000528202.5 (SEQ ID NO: 10353); |
| | ENST00000528374.1 (SEQ ID NO: 10355); ENST00000529967.5 (SEQ ID NO: 10357); |
| | ENST00000530147.5 (SEQ ID NO: 10359); ENST00000531137.1 (SEQ ID NO: 10361); |
| | ENST00000531642.5 (SEQ ID NO: 10363); ENST00000531891.1 (SEQ ID NO: 10365); |
| | ENST00000531911.1 (SEQ ID NO: 10367); ENST00000532220.1 (SEQ ID NO: 10369); |
| | ENST00000532728.6 (SEQ ID NO: 15308); ENST00000612903.1 (SEQ ID NO: 10372); |
| | ENST00000635881.1 (SEQ ID NO: 15309); ENST00000642271.1 (SEQ ID NO: 15310); |
| | ENST00000642579.1 (SEQ ID NO: 15311); ENST00000642611.1 (SEQ ID NO: 15312); |
| | ENST00000642902.1 (SEQ ID NO: 15313); ENST00000643260.1 (SEQ ID NO: 15314); |
| | ENST00000643562.1 (SEQ ID NO: 15315); ENST00000643925.1 (SEQ ID NO: 15316); |
| | ENST00000644057.1 (SEQ ID NO: 10382); ENST00000644447.1 (SEQ ID NO: 15317); |
| | ENST00000644472.1 (SEQ ID NO: 15318); ENST00000644484.1 (SEQ ID NO: 15319); |
| | ENST00000644542.1 (SEQ ID NO: 15320); ENST00000644649.1 (SEQ ID NO: 15321); |
| | ENST00000644675.1 (SEQ ID NO: 15322); ENST00000644757.1 (SEQ ID NO: 15323); |
| | ENST00000644772.1 (SEQ ID NO: 15324); ENST00000645004.1 (SEQ ID NO: 10392); |
| | ENST00000645076.1 (SEQ ID NO: 15325); ENST00000645417.1 (SEQ ID NO: 10395); |
| | ENST00000645744.1 (SEQ ID NO: 15326); ENST00000645760.1 (SEQ ID NO: 15327); |
| | ENST00000645884.1 (SEQ ID NO: 15328); ENST00000646003.1 (SEQ ID NO: 15329); |
| | ENST00000646207.1 (SEQ ID NO: 15330); ENST00000646276.1 (SEQ ID NO: 15331); |
| | ENST00000646592.1 (SEQ ID NO: 15332); ENST00000646737.1 (SEQ ID NO: 10404); |
| | ENST00000646902.1 (SEQ ID NO: 15333); ENST00000646993.1 (SEQ ID NO: 15334); |
| | ENST00000647013.1 (SEQ ID NO: 15335); ENST00000647015.1 (SEQ ID NO: 15336); |
| | ENST00000647086.1 (SEQ ID NO: 15337); ENST00000647158.1 (SEQ ID NO: 15338) |
| transcript | ENST00000302539.9 (SEQ ID NO: 10338); ENST00000389817.7 (SEQ ID NO: 10339); |
| | ENST00000524561.1 (SEQ ID NO: 10341); ENST00000525022.1 (SEQ ID NO: 10343); |
| | ENST00000526002.1 (SEQ ID NO: 10345); ENST00000526037.5 (SEQ ID NO: 10347); |
| | ENST00000526168.5 (SEQ ID NO: 10349); ENST00000526921.5 (SEQ ID NO: 10351); |
| | ENST00000527905.5 (SEQ ID NO: 10352); ENST00000528202.5 (SEQ ID NO: 10354); |
| | ENST00000528374.1 (SEQ ID NO: 10356); ENST00000529967.5 (SEQ ID NO: 10358); |
| | ENST00000530147.5 (SEQ ID NO: 10360); ENST00000531137.1 (SEQ ID NO: 10362); |
| | ENST00000531642.5 (SEQ ID NO: 10364); ENST00000531891.1 (SEQ ID NO: 10366); |
| | ENST00000531911.1 (SEQ ID NO: 10368); ENST00000532220.1 (SEQ ID NO: 10370); |
| | ENST00000532728.6 (SEQ ID NO: 10371); ENST00000612903.1 (SEQ ID NO: 10373); |
| | ENST00000635881.1 (SEQ ID NO: 10374); ENST00000642271.1 (SEQ ID NO: 10375); |
| | ENST00000642579.1 (SEQ ID NO: 10376); ENST00000642611.1 (SEQ ID NO: 10377); |
| | ENST00000642902.1 (SEQ ID NO: 10378); ENST00000643260.1 (SEQ ID NO: 10379); |
| | ENST00000643562.1 (SEQ ID NO: 10380); ENST00000643925.1 (SEQ ID NO: 10381); |
| | ENST00000644057.1 (SEQ ID NO: 10383); ENST00000644447.1 (SEQ ID NO: 10384); |
| | ENST00000644472.1 (SEQ ID NO: 10385); ENST00000644484.1 (SEQ ID NO: 10386); |
| | ENST00000644542.1 (SEQ ID NO: 10387); ENST00000644649.1 (SEQ ID NO: 10388); |
| | ENST00000644675.1 (SEQ ID NO: 10389); ENST00000644757.1 (SEQ ID NO: 10390); |
| | ENST00000644772.1 (SEQ ID NO: 10391); ENST00000645004.1 (SEQ ID NO: 10393); |
| | ENST00000645076.1 (SEQ ID NO: 10394); ENST00000645417.1 (SEQ ID NO: 10396); |
| | ENST00000645744.1 (SEQ ID NO: 10397); ENST00000645760.1 (SEQ ID NO: 10398); |
| | ENST00000645884.1 (SEQ ID NO: 10399); ENST00000646003.1 (SEQ ID NO: 10400); |
| | ENST00000646207.1 (SEQ ID NO: 10401); ENST00000646276.1 (SEQ ID NO: 10402); |
| | ENST00000646592.1 (SEQ ID NO: 10403); ENST00000646737.1 (SEQ ID NO: 10405); |
| | ENST00000646902.1 (SEQ ID NO: 10406); ENST00000646993.1 (SEQ ID NO; 10407); |
| | ENST00000647013.1 (SEQ ID NO: 10408); ENST00000647015.1 (SEQ ID NO: 10409); |
| | ENST00000647086.1 (SEQ ID NO: 10410); ENST00000647158.1 (SEQ ID NO: 10411) |

Target: ABCD1

| | |
|---|---|
| pre-mRNA | ENST00000218104.6 (SEQ ID NO: 10412); ENST00000370129.4 (SEQ ID NO: 10414); |
| transcript | ENST00000443684.2 (SEQ ID NO: 10416) |
| transcript | ENST00000218104.6 (SEQ ID NO: 10413); ENST00000370129.4 (SEQ ID NO: 10415); |
| | ENST00000443684.2 (SEQ ID NO: 10417) |

Target: ABR

| | |
|---|---|
| pre-mRNA | ENST00000291107.6 (SEQ ID NO: 15339); ENST00000302538.9 (SEQ ID NO: 15340); |
| transcript | ENST00000536794.6 (SEQ ID NO: 15341); ENST00000543210.6 (SEQ ID NO: 10421); |
| | ENST00000544583.6 (SEQ ID NO: 15342); ENST00000570441.5 (SEQ ID NO: 15343); |
| | ENST00000570525.5 (SEQ ID NO: 15344); ENST00000570688.1 (SEQ ID NO: 10426); |
| | ENST00000571022.5 (SEQ ID NO: 10428); ENST00000571120.5 (SEQ ID NO: 10430); |
| | ENST00000571306.1 (SEQ ID NO: 10432); ENST00000571383.5 (SEQ ID NO: 10434); |
| | ENST00000571543.1 (SEQ ID NO: 10436); ENST00000571797.5 (SEQ ID NO: 10438); |
| | ENST00000571945.5 (SEQ ID NO: 10440); ENST00000572152.5 (SEQ ID NO: 15345); |
| | ENST00000572441.5 (SEQ ID NO: 10443); ENST00000572585.5 (SEQ ID NO: 10445); |
| | ENST00000572650.1 (SEQ ID NO: 10447); ENST00000573325.1 (SEQ ID NO: 10449); |
| | ENST00000573559.3 (SEQ ID NO: 10451); ENST00000573667.1 (SEQ ID NO: 10453); |
| | ENST00000573895.1 (SEQ ID NO: 10455); ENST00000574048.2 (SEQ ID NO: 10457); |
| | ENST00000574139.6 (SEQ ID NO: 15346); ENST00000574257.5 (SEQ ID NO: 15347); |
| | ENST00000574266.1 (SEQ ID NO: 15348); ENST00000574437.5 (SEQ ID NO: 15349); |
| | ENST00000574544.1 (SEQ ID NO: 10463); ENST00000574632.5 (SEQ ID NO: 10465); |
| | ENST00000574875.1 (SEQ ID NO: 10467); ENST00000575770.5 (SEQ ID NO: 10469); |
| | ENST00000575934.5 (SEQ ID NO: 15350); ENST00000576668.1 (SEQ ID NO: 10472); |
| | ENST00000576964.5 (SEQ ID NO: 15351); ENST00000577052.5 (SEQ ID NO: 10475) |
| transcript | ENST00000291107.6 (SEQ ID NO: 10418); ENST00000302538.9 (SEQ ID NO: 10419); |
| | ENST00000536794.6 (SEQ ID NO: 10420); ENST00000543210.6 (SEQ ID NO: 10422); |
| | ENST00000544583.6 (SEQ ID NO: 10423); ENST00000570441.5 (SEQ ID NO: 10424); |
| | ENST00000570525.5 (SEQ ID NO: 10425); ENST00000570688.1 (SEQ ID NO: 10427); |
| | ENST00000571022.5 (SEQ ID NO: 10429); ENST00000571120.5 (SEQ ID NO: 10431); |
| | ENST00000571306.1 (SEQ ID NO: 10433); ENST00000571383.5 (SEQ ID NO: 10435); |
| | ENST00000571543.1 (SEQ ID NO: 10437); ENST00000571797.5 (SEQ ID NO: 10439); |
| | ENST00000571945.5 (SEQ ID NO: 10441); ENST00000572152.5 (SEQ ID NO: 10442); |
| | ENST00000572441.5 (SEQ ID NO: 10444); ENST00000572585.5 (SEQ ID NO: 10446); |
| | ENST00000572650.1 (SEQ ID NO: 10448); ENST00000573325.1 (SEQ ID NO: 10450); |
| | ENST00000573559.3 (SEQ ID NO: 10452); ENST00000573667.1 (SEQ ID NO: 10454); |
| | ENST00000573895.1 (SEQ ID NO: 10456); ENST00000574048.2 (SEQ ID NO: 10458); |
| | ENST00000574139.6 (SEQ ID NO: 10459); ENST00000574257.5 (SEQ ID NO: 10460); |
| | ENST00000574266.1 (SEQ ID NO: 10461); ENST00000574437.5 (SEQ ID NO: 10462); |
| | ENST00000574544.1 (SEQ ID NO: 10464); ENST00000574632.5 (SEQ ID NO: 10466); |
| | ENST00000574875.1 (SEQ ID NO: 10468); ENST00000575770.5 (SEQ ID NO: 10470); |
| | ENST00000575934.5 (SEQ ID NO: 10471); ENST00000576668.1 (SEQ ID NO: 10473); |
| | ENST00000576964.5 (SEQ ID NO: 10474); ENST00000577052.5 (SEQ ID NO: 10476) |

Target: ACAD9

| | |
|---|---|
| pre-mRNA | ENST00000308982.12 (SEQ ID NO: 15352); ENST00000505192.5 (SEQ ID NO: 10478); |
| transcript | ENST00000505602.1 (SEQ ID NO: 10480); ENST00000505867.5 (SEQ ID NO: 15353); |
| | ENST00000508971.1 (SEQ ID NO: 10483); ENST00000511227.5 (SEQ ID NO: 15354); |
| | ENST00000511325.1 (SEQ ID NO: 10486); ENST00000511526.5 (SEQ ID NO: 10488); |
| | ENST00000512801.5 (SEQ ID NO: 10490); ENST00000514336.1 (SEQ ID NO: 10492); |
| | ENST00000514643.5 (SEQ ID NO: 10494); ENST00000515429.1 (SEQ ID NO: 10496) |
| transcript | ENST00000308982.12 (SEQ ID NO: 10477); ENST00000505192.5 (SEQ ID NO: 10479); |
| | ENST00000505602.1 (SEQ ID NO: 10481); ENST00000505867.5 (SEQ ID NO: 10482); |
| | ENST00000508971.1 (SEQ ID NO: 10484); ENST00000511227.5 (SEQ ID NO: 10485); |
| | ENST00000511325.1 (SEQ ID NO: 10487); ENST00000511526.5 (SEQ ID NO: 10489); |
| | ENST00000512801.5 (SEQ ID NO: 10491); ENST00000514336.1 (SEQ ID NO: 10493); |
| | ENST00000514643.5 (SEQ ID NO: 10495); ENST00000515429.1 (SEQ ID NO: 10497) |

Target: ACAP1

| | |
|---|---|
| pre-mRNA | premrna_ENST00000158762.8; premrna_ENST00000570439.1; premrna_ENST00000570457.6; |
| transcript | premrna_ENST00000570504.5; premrna_ENST00000571220.1; premrna_ENST00000571471.5; |
| | premrna_ENST00000573893.5; premrna_ENST00000574499.1; premrna_ENST00000575415.1; |
| | premrna_ENST00000575425.1; premrna_ENST00000576594.1; premrna_ENST00000576628.1; |
| transcript | transcript_ENST00000158762.8; transcript_ENST00000570439.1; transcript_ENST00000570457.6; |
| | transcript_ENST00000570504.5; transcript_ENST00000571220.1; transcript_ENST00000571471.5; |
| | transcript_ENST00000573893.5; transcript_ENST00000574499.1; transcript_ENST00000575415.1; |
| | transcript_ENST00000575425.1; transcript_ENST00000576594.1; transcript_ENST00000576628.1 |

Target: ACOX2

| | |
|---|---|
| pre-mRNA | ENST00000302819.10 (SEQ ID NO: 15355); ENST00000459701.6 (SEQ ID NO: 15356); |
| transcript | ENST00000459888.1 (SEQ ID NO: 10500); ENST00000460921.1 (SEQ ID NO: 10502); |
| | ENST00000466689.1 (SEQ ID NO: 10504); ENST00000466810.5 (SEQ ID NO: 10506); |
| | ENST00000467738.1 (SEQ ID NO: 10508); ENST00000474098.1 (SEQ ID NO: 10510); |
| | ENST00000475143.5 (SEQ ID NO: 10512); ENST00000480791.1 (SEQ ID NO: 10514); |
| | ENST00000481527.5 (SEQ ID NO: 10516); ENST00000489472.1 (SEQ ID NO: 10518); |
| | ENST00000492530.1 (SEQ ID NO: 10520) |
| transcript | ENST00000302819.10 (SEQ ID NO: 10498); ENST00000459701.6 (SEQ ID NO: 10499); |
| | ENST00000459888.1 (SEQ ID NO: 10501); ENST00000460921.1 (SEQ ID NO: 10503); |
| | ENST00000466689.1 (SEQ ID NO: 10505); ENST00000466810.5 (SEQ ID NO: 10507); |
| | ENST00000467738.1 (SEQ ID NO: 10509); ENST00000474098.1 (SEQ ID NO: 10511); |
| | ENST00000475143.5 (SEQ ID NO: 10513); ENST00000480791.1 (SEQ ID NO: 10515); |
| | ENST00000481527.5 (SEQ ID NO: 10517); ENST00000489472.1 (SEQ ID NO: 10519); |
| | ENST00000492530.1 (SEQ ID NO: 10521) |

Target: ACSF3

| | |
|---|---|
| pre-mRNA | ENST00000317447.9 (SEQ ID NO: 15357); ENST00000378345.8 (SEQ ID NO: 15358); |
| transcript | ENST00000393145.5 (SEQ ID NO: 10524); ENST00000406948.7 (SEQ ID NO: 15359); |
| | ENST00000535176.1 (SEQ ID NO: 10527); ENST00000537116.5 (SEQ ID NO: 10529); |
| | ENST00000537155.1 (SEQ ID NO: 10531); ENST00000537290.5 (SEQ ID NO: 10533); |
| | ENST00000537895.5 (SEQ ID NO: 10535); ENST00000538340.5 (SEQ ID NO: 10537); |
| | ENST00000540697.5 (SEQ ID NO: 15360); ENST00000541755.2 (SEQ ID NO: 10540); |
| | ENST00000542688.5 (SEQ ID NO: 15361); ENST00000543676.1 (SEQ ID NO: 10543); |
| | ENST00000544543.5 (SEQ ID NO: 15362); ENST00000562204.1 (SEQ ID NO: 15363); |
| | ENST00000562750.1 (SEQ ID NO: 10547); ENST00000614302.5 (SEQ ID NO: 15364); |
| | ENST00000649953.1 (SEQ ID NO: 15365) |
| transcript | ENST00000317447.9 (SEQ ID NO: 10522); ENST00000378345.8 (SEQ ID NO: 10523); |
| | ENST00000393145.5 (SEQ ID NO: 10525); ENST00000406948.7 (SEQ ID NO: 10526); |
| | ENST00000535176.1 (SEQ ID NO: 10528); ENST00000537116.5 (SEQ ID NO: 10530); |
| | ENST00000537155.1 (SEQ ID NO: 10532); ENST00000537290.5 (SEQ ID NO: 10534); |
| | ENST00000537895.5 (SEQ ID NO: 10536); ENST00000538340.5 (SEQ ID NO: 10538); |
| | ENST00000540697.5 (SEQ ID NO: 10539); ENST00000541755.2 (SEQ ID NO: 10541); |
| | ENST00000542688.5 (SEQ ID NO: 10542); ENST00000543676.1 (SEQ ID NO: 10544); |
| | ENST00000544543.5 (SEQ ID NO: 10545); ENST00000562204.1 (SEQ ID NO: 10546); |
| | ENST00000562750.1 (SEQ ID NO: 10548); ENST00000614302.5 (SEQ ID NO: 10549); |
| | ENST00000649953.1 (SEQ ID NO: 10550) |

Target: ACTN4

| | |
|---|---|
| pre-mRNA | ENST00000252699.7 (SEQ ID NO: 15366); ENST00000390009.7 (SEQ ID NO: 15367); |
| transcript | ENST00000424234.6 (SEQ ID NO: 15368); ENST00000440400.1 (SEQ ID NO: 10554); |
| | ENST00000477174.1 (SEQ ID NO: 10556); ENST00000489451.1 (SEQ ID NO: 10558); |
| | ENST00000495553.1 (SEQ ID NO: 15369); ENST00000497637.5 (SEQ ID NO: 10561); |
| | ENST00000586538.1 (SEQ ID NO: 10563); ENST00000588618.5 (SEQ ID NO: 15370); |
| | ENST00000589528.1 (SEQ ID NO: 15371) |
| transcript | ENST00000252699.7 (SEQ ID NO: 10551); ENST00000390009.7 (SEQ ID NO: 10552); |
| | ENST00000424234.6 (SEQ ID NO: 10553); ENST00000440400.1 (SEQ ID NO: 10555); |
| | ENST00000477174.1 (SEQ ID NO: 10557); ENST00000489451.1 (SEQ ID NO: 10559); |
| | ENST00000495553.1 (SEQ ID NO: 10560); ENST00000497637.5 (SEQ ID NO: 10562); |
| | ENST00000586538.1 (SEQ ID NO: 10564); ENST00000588618.5 (SEQ ID NO: 10565); |
| | ENST00000589528.1 (SEQ ID NO: 10566) |

Target: ADAM17

| | |
|---|---|
| pre-mRNA | ENST00000310823.8 (SEQ ID NO: 15372); ENST00000478059.1 (SEQ ID NO: 10568); |
| transcript | ENST00000618923.2 (SEQ ID NO: 15373); ENST00000647610.1 (SEQ ID NO: 15374); |
| | ENST00000647622.1 (SEQ ID NO: 15375); ENST00000647979.1 (SEQ ID NO: 15376); |
| | ENST00000648002.1 (SEQ ID NO: 10574); ENST00000648548.1 (SEQ ID NO: 15377); |
| | ENST00000648857.1 (SEQ ID NO: 15378); ENST00000649068.1 (SEQ ID NO: 10578); |
| | ENST00000649227.1 (SEQ ID NO: 15379); ENST00000649686.1 (SEQ ID NO: 15380); |
| | ENST00000649798.1 (SEQ ID NO: 10582); ENST00000649972.1 (SEQ ID NO: 15381); |
| | ENST00000650116.1 (SEQ ID NO: 15382); ENST00000650241.1 (SEQ ID NO: 15383) |
| transcript | ENST00000310823.8 (SEQ ID NO: 10567); ENST00000478059.1 (SEQ ID NO: 10569); |
| | ENST00000618923.2 (SEQ ID NO: 10570); ENST00000647610.1 (SEQ ID NO: 10571); |
| | ENST00000647622.1 (SEQ ID NO: 10572); ENST00000647979.1 (SEQ ID NO: 10573); |
| | ENST00000648002.1 (SEQ ID NO: 10575); ENST00000648548.1 (SEQ ID NO: 10576); |
| | ENST00000648857.1 (SEQ ID NO: 10577); ENST00000649068.1 (SEQ ID NO: 10579); |
| | ENST00000649227.1 (SEQ ID NO: 10580); ENST00000649686.1 (SEQ ID NO: 10581); |
| | ENST00000649798.1 (SEQ ID NO: 10583); ENST00000649972.1 (SEQ ID NO: 10584); |
| | ENST00000650116.1 (SEQ ID NO: 10585); ENST00000650241.1 (SEQ ID NO: 10586) |

Target: ADAMTS13

| | |
|---|---|
| pre-mRNA | ENST00000355699.6 (SEQ ID NO: 15384); ENST00000356589.6 (SEQ ID NO: 15385); |
| transcript | ENST00000371910.1 (SEQ ID NO: 10589); ENST00000371911.7 (SEQ ID NO: 10591); |
| | ENST00000371916.5 (SEQ ID NO: 15386); ENST00000371929.7 (SEQ ID NO: 15387); |
| | ENST00000474918.1 (SEQ ID NO: 10595); ENST00000485925.5 (SEQ ID NO: 15388); |
| | ENST00000495234.5 (SEQ ID NO: 10598) |
| transcript | ENST00000355699.6 (SEQ ID NO: 10587); ENST00000356589.6 (SEQ ID NO: 10588); |
| | ENST00000371910.1 (SEQ ID NO: 10590); ENST00000371911.7 (SEQ ID NO: 10592); |
| | ENST00000371916.5 (SEQ ID NO: 10593); ENST00000371929.7 (SEQ ID NO: 10594); |
| | ENST00000474918.1 (SEQ ID NO: 10596); ENST00000485925.5 (SEQ ID NO: 10597); |
| | ENST00000495234.5 (SEQ ID NO: 10599) |

Target: AKR1E2

| | |
|---|---|
| pre-mRNA | ENST00000298375.12 (SEQ ID NO: 10600); ENST00000334019.4 (SEQ ID NO: 10602); |
| transcript | ENST00000345253.9 (SEQ ID NO: 10604); ENST00000441590.5 (SEQ ID NO: 10606); |
| | ENST00000462718.7 (SEQ ID NO: 10608); ENST00000463345.5 (SEQ ID NO: 10610); |
| | ENST00000474119.5 (SEQ ID NO: 10612); ENST00000487985.1 (SEQ ID NO: 10614); |
| | ENST00000525281.5 (SEQ ID NO: 10616); ENST00000525572.1 (SEQ ID NO: 10618); |
| | ENST00000525627.1 (SEQ ID NO: 10620); ENST00000532248.5 (SEQ ID NO: 10622); |
| | ENST00000533295.5 (SEQ ID NO: 10624) |
| transcript | ENST00000298375.12 (SEQ ID NO: 10601); ENST00000334019.4 (SEQ ID NO: 10603); |
| | ENST00000345253.9 (SEQ ID NO: 10605); ENST00000441590.5 (SEQ ID NO: 10607); |
| | ENST00000462718.7 (SEQ ID NO: 10609); ENST00000463345.5 (SEQ ID NO: 10611); |
| | ENST00000474119.5 (SEQ ID NO: 10613); ENST00000487985.1 (SEQ ID NO: 10615); |
| | ENST00000525281.5 (SEQ ID NO: 10617); ENST00000525572.1 (SEQ ID NO: 10619); |
| | ENST00000525627.1 (SEQ ID NO: 10621); ENST00000532248.5 (SEQ ID NO: 10623); |
| | ENST00000533295.5 (SEQ ID NO: 10625) |

Target: ALAD

| | |
|---|---|
| pre-mRNA | ENST00000409155.8 (SEQ ID NO: 10626); ENST00000445750.1 (SEQ ID NO: 10628); |
| transcript | ENST00000448137.5 (SEQ ID NO: 10630); ENST00000452726.1 (SEQ ID NO: 10632); |
| | ENST00000464749.5 (SEQ ID NO: 10634); ENST00000468504.5 (SEQ ID NO: 10636); |
| | ENST00000482001.1 (SEQ ID NO: 10638); ENST00000482847.5 (SEQ ID NO: 10640); |
| | ENST00000494848.1 (SEQ ID NO: 10642) |
| transcript | ENST00000409155.8 (SEQ ID NO: 10627); ENST00000445750.1 (SEQ ID NO: 10629); |
| | ENST00000448137.5 (SEQ ID NO: 10631); ENST00000452726.1 (SEQ ID NO: 10633); |
| | ENST00000464749.5 (SEQ ID NO: 10635); ENST00000468504.5 (SEQ ID NO: 10637); |
| | ENST00000482001.1 (SEQ ID NO: 10639); ENST00000482847.5 (SEQ ID NO: 10641); |
| | ENST00000494848.1 (SEQ ID NO: 10643) |

Target: ALG3

| | |
|---|---|
| pre-mRNA | ENST00000397676.8 (SEQ ID NO: 10644); ENST00000411922.5 (SEQ ID NO: 10646); |
| transcript | ENST00000414845.5 (SEQ ID NO: 10648); ENST00000423996.5 (SEQ ID NO: 10650); |
| | ENST00000445626.6 (SEQ ID NO: 10652); ENST00000446569.1 (SEQ ID NO: 10654); |
| | ENST00000455059.5 (SEQ ID NO: 10656); ENST00000461415.5 (SEQ ID NO: 10658); |
| | ENST00000462735.6 (SEQ ID NO: 10660); ENST00000463495.5 (SEQ ID NO: 10662); |
| | ENST00000477959.1 (SEQ ID NO: 10664); ENST00000482048.1 (SEQ ID NO: 10666); |
| | ENST00000485912.1 (SEQ ID NO: 10668); ENST00000488976.5 (SEQ ID NO: 10670) |
| transcript | ENST00000397676.8 (SEQ ID NO: 10645); ENST00000411922.5 (SEQ ID NO: 10647); |
| | ENST00000414845.5 (SEQ ID NO: 10649); ENST00000423996.5 (SEQ ID NO: 10651); |
| | ENST00000445626.6 (SEQ ID NO: 10653); ENST00000446569.1 (SEQ ID NO: 10655); |
| | ENST00000455059.5 (SEQ ID NO: 10657); ENST00000461415.5 (SEQ ID NO: 10659); |
| | ENST00000462735.6 (SEQ ID NO: 10661); ENST00000463495.5 (SEQ ID NO: 10663); |
| | ENST00000477959.1 (SEQ ID NO: 10665); ENST00000482048.1 (SEQ ID NO: 10667); |
| | ENST00000485912.1 (SEQ ID NO: 10669); ENST00000488976.5 (SEQ ID NO: 10671) |

Target: ANKRD29

| | |
|---|---|
| pre-mRNA | ENST00000322980.13 (SEQ ID NO: 15389); ENST00000585908.2 (SEQ ID NO: 10673); |
| transcript | ENST00000586087.1 (SEQ ID NO: 15390); ENST00000586511.1 (SEQ ID NO: 10676); |
| | ENST00000587763.1 (SEQ ID NO: 10678); ENST00000591280.5 (SEQ ID NO: 15391); |
| | ENST00000591617.1 (SEQ ID NO: 10681); ENST00000592179.6 (SEQ ID NO: 15392) |
| transcript | ENST00000322980.13 (SEQ ID NO: 10672); ENST00000585908.2 (SEQ ID NO: 10674); |
| | ENST00000586087.1 (SEQ ID NO: 10675); ENST00000586511.1 (SEQ ID NO: 10677); |
| | ENST00000587763.1 (SEQ ID NO: 10679); ENST00000591280.5 (SEQ ID NO: 10680); |
| | ENST00000591617.1 (SEQ ID NO: 10682); ENST00000592179.6 (SEQ ID NO: 10683) |

Target: ANKS3

| | |
|---|---|
| pre-mRNA | ENST00000304283.9 (SEQ ID NO: 15393); ENST00000446014.6 (SEQ ID NO: 15394); |
| transcript | ENST00000450067.6 (SEQ ID NO: 15395); ENST00000585773.5 (SEQ ID NO: 15396); |
| | ENST00000586159.5 (SEQ ID NO: 10688); ENST00000586166.5 (SEQ ID NO: 15397); |
| | ENST00000586605.5 (SEQ ID NO: 15398); ENST00000586632.1 (SEQ ID NO: 10692); |
| | ENST00000587005.5 (SEQ ID NO: 10694); ENST00000588398.1 (SEQ ID NO: 10696); |
| | ENST00000588513.1 (SEQ ID NO: 10698); ENST00000589035.5 (SEQ ID NO: 10700); |
| | ENST00000589065.5 (SEQ ID NO: 10702); ENST00000590147.5 (SEQ ID NO: 10704); |
| | ENST00000590193.5 (SEQ ID NO: 15399); ENST00000590689.1 (SEQ ID NO: 10707); |
| | ENST00000590730.5 (SEQ ID NO: 15400); ENST00000590803.5 (SEQ ID NO: 15401); |
| | ENST00000591185.5 (SEQ ID NO: 10711); ENST00000591281.5 (SEQ ID NO: 10713); |
| | ENST00000591653.5 (SEQ ID NO: 15402); ENST00000592068.5 (SEQ ID NO: 15403); |
| | ENST00000592077.5 (SEQ ID NO: 15404); ENST00000592190.1 (SEQ ID NO: 10718); |
| | ENST00000592421.5 (SEQ ID NO: 15405); ENST00000592698.5 (SEQ ID NO: 15406); |
| | ENST00000592711.5 (SEQ ID NO: 10722); ENST00000592840.1 (SEQ ID NO: 10724); |
| | ENST00000593120.5 (SEQ ID NO: 10726); ENST00000614075.4 (SEQ ID NO: 15407) |
| transcript | ENST00000304283.9 (SEQ ID NO: 10684); ENST00000446014.6 (SEQ ID NO: 10685); |
| | ENST00000450067.6 (SEQ ID NO: 10686); ENST00000585773.5 (SEQ ID NO: 10687); |
| | ENST00000586159.5 (SEQ ID NO: 10689); ENST00000586166.5 (SEQ ID NO: 10690); |
| | ENST00000586605.5 (SEQ ID NO: 10691); ENST00000586632.1 (SEQ ID NO: 10693); |
| | ENST00000587005.5 (SEQ ID NO: 10695); ENST00000588398.1 (SEQ ID NO: 10697); |
| | ENST00000588513.1 (SEQ ID NO: 10699); ENST00000589035.5 (SEQ ID NO: 10701); |
| | ENST00000589065.5 (SEQ ID NO: 10703); ENST00000590147.5 (SEQ ID NO: 10705); |
| | ENST00000590193.5 (SEQ ID NO: 10706); ENST00000590689.1 (SEQ ID NO: 10708); |
| | ENST00000590730.5 (SEQ ID NO: 10709); ENST00000590803.5 (SEQ ID NO: 10710); |
| | ENST00000591185.5 (SEQ ID NO: 10712); ENST00000591281.5 (SEQ ID NO: 10714); |
| | ENST00000591653.5 (SEQ ID NO: 10715); ENST00000592068.5 (SEQ ID NO: 10716); |
| | ENST00000592077.5 (SEQ ID NO: 10717); ENST00000592190.1 (SEQ ID NO: 10719); |
| | ENST00000592421.5 (SEQ ID NO: 10720); ENST00000592698.5 (SEQ ID NO: 10721); |
| | ENST00000592711.5 (SEQ ID NO: 10723); ENST00000592840.1 (SEQ ID NO: 10725); |
| | ENST00000593120.5 (SEQ ID NO: 10727); ENST00000614075.4 (SEQ ID NO: 10728) |

Target: ANO4

| | |
|---|---|
| pre-mRNA | ENST00000392977.8 (SEQ ID NO: 15408); ENST00000392979.7 (SEQ ID NO: 15409); |
| transcript | ENST00000546991.1 (SEQ ID NO: 15410); ENST00000548940.1 (SEQ ID NO: 15411); |
| | ENST00000549155.6 (SEQ ID NO: 15412); ENST00000549234.1 (SEQ ID NO: 15413); |
| | ENST00000550015.1 (SEQ ID NO: 15414); ENST00000551148.1 (SEQ ID NO: 15415); |
| | ENST00000644049.1 (SEQ ID NO: 15416) |
| transcript | ENST00000392977.8 (SEQ ID NO: 10729); ENST00000392979.7 (SEQ ID NO: 10730); |
| | ENST00000546991.1 (SEQ ID NO: 10731); ENST00000548940.1 (SEQ ID NO: 10732); |
| | ENST00000549155.6 (SEQ ID NO: 10733); ENST00000549234.1 (SEQ ID NO: 10734); |
| | ENST00000550015.1 (SEQ ID NO: 10735); ENST00000551148.1 (SEQ ID NO: 10736); |
| | ENST00000644049.1 (SEQ ID NO: 10737) |

Target: AP3B2

| | |
|---|---|
| pre-mRNA | ENST00000261722.8 (SEQ ID NO: 15417); ENST00000535348.5 (SEQ ID NO: 15418); |
| transcript | ENST00000535359.6 (SEQ ID NO: 15419); ENST00000535385.6 (SEQ ID NO: 15420); |
| | ENST00000535513.2 (SEQ ID NO: 10742); ENST00000537735.2 (SEQ ID NO: 15421); |
| | ENST00000541693.5 (SEQ ID NO: 10745); ENST00000542200.2 (SEQ ID NO: 10747); |
| | ENST00000543938.6 (SEQ ID NO: 15422); ENST00000559888.1 (SEQ ID NO: 10750); |
| | ENST00000560529.1 (SEQ ID NO: 10752); ENST00000561455.5 (SEQ ID NO: 10754); |
| | ENST00000620652.4 (SEQ ID NO: 15423); ENST00000642989.2 (SEQ ID NO: 15424); |
| | ENST00000652847.1 (SEQ ID NO: 15425); ENST00000657321.1 (SEQ ID NO: 15426); |
| | ENST00000659252.1 (SEQ ID NO: 10760); ENST00000660624.1 (SEQ ID NO: 10762); |
| | ENST00000661532.1 (SEQ ID NO: 10764); ENST00000663651.1 (SEQ ID NO: 15427); |
| | ENST00000664460.1 (SEQ ID NO: 15428); ENST00000665513.1 (SEQ ID NO: 10768); |
| | ENST00000666055.1 (SEQ ID NO: 15429); ENST00000666672.1 (SEQ ID NO: 10771); |
| | ENST00000666894.1 (SEQ ID NO: 10773); ENST00000666973.1 (SEQ ID NO: 15430); |
| | ENST00000667758.1 (SEQ ID NO: 15431); ENST00000668385.1 (SEQ ID NO: 15432); |
| | ENST00000668458.1 (SEQ ID NO: 10778); ENST00000668990.2 (SEQ ID NO: 15433); |
| | ENST00000669880.1 (SEQ ID NO: 10781); ENST00000669930.1 (SEQ ID NO: 15434) |
| transcript | ENST00000261722.8 (SEQ ID NO: 10738); ENST00000535348.5 (SEQ ID NO: 10739); |
| | ENST00000535359.6 (SEQ ID NO: 10740); ENST00000535385.6 (SEQ ID NO: 10741); |
| | ENST00000535513.2 (SEQ ID NO: 10743); ENST00000537735.2 (SEQ ID NO: 10744); |
| | ENST00000541693.5 (SEQ ID NO: 10746); ENST00000542200.2 (SEQ ID NO: 10748); |
| | ENST00000543938.6 (SEQ ID NO: 10749); ENST00000559888.1 (SEQ ID NO: 10751); |
| | ENST00000560529.1 (SEQ ID NO: 10753); ENST00000561455.5 (SEQ ID NO: 10755); |
| | ENST00000620652.4 (SEQ ID NO: 10756); ENST00000642989.2 (SEQ ID NO: 10757); |
| | ENST00000652847.1 (SEQ ID NO: 10758); ENST00000657321.1 (SEQ ID NO: 10759); |
| | ENST00000659252.1 (SEQ ID NO: 10761); ENST00000660624.1 (SEQ ID NO: 10763); |
| | ENST00000661532.1 (SEQ ID NO: 10765); ENST00000663651.1 (SEQ ID NO: 10766); |
| | ENST00000664460.1 (SEQ ID NO: 10767); ENST00000665513.1 (SEQ ID NO: 10769); |
| | ENST00000666055.1 (SEQ ID NO: 10770); ENST00000666672.1 (SEQ ID NO: 10772); |
| | ENST00000666894.1 (SEQ ID NO: 10774); ENST00000666973.1 (SEQ ID NO: 10775); |
| | ENST00000667758.1 (SEQ ID NO: 10776); ENST00000668385.1 (SEQ ID NO: 10777); |
| | ENST00000668458.1 (SEQ ID NO: 10779); ENST00000668990.2 (SEQ ID NO: 10780); |
| | ENST00000669880.1 (SEQ ID NO: 10782); ENST00000669930.1 (SEQ ID NO: 10783) |

Target: AP3M1

| | |
|---|---|
| pre-mRNA | ENST00000355264.8 (SEQ ID NO: 15435); ENST00000372745.1 (SEQ ID NO: 10785); |
| transcript | ENST00000480373.1 (SEQ ID NO: 10787); ENST00000487653.1 (SEQ ID NO: 10789) |
| transcript | ENST00000355264.8 (SEQ ID NO: 10784); ENST00000372745.1 (SEQ ID NO: 10786); |
| | ENST00000480373.1 (SEQ ID NO: 10788); ENST00000487653.1 (SEQ ID NO: 10790) |

Target: AP5Z1

| | |
|---|---|
| pre-mRNA | ENST00000469614.1 (SEQ ID NO: 10791); ENST00000477454.1 (SEQ ID NO: 10793); |
| transcript | ENST00000477680.6 (SEQ ID NO: 10795); ENST00000490487.1 (SEQ ID NO: 10797); |
| | ENST00000491375.1 (SEQ ID NO: 10799); ENST00000496303.6 (SEQ ID NO: 10801); |
| | ENST00000647628.1 (SEQ ID NO: 10803); ENST00000647984.1 (SEQ ID NO: 10805); |
| | ENST00000648237.1 (SEQ ID NO: 10807); ENST00000648360.1 (SEQ ID NO: 10809); |
| | ENST00000648765.1 (SEQ ID NO: 10811); ENST00000648925.1 (SEQ ID NO: 10813); |
| | ENST00000649063.2 (SEQ ID NO: 10815); ENST00000649315.1 (SEQ ID NO: 10817); |
| | ENST00000649419.1 (SEQ ID NO: 10819); ENST00000649736.1 (SEQ ID NO: 10821); |
| | ENST00000650310.1 (SEQ ID NO: 10823); ENST00000650451.1 (SEQ ID NO: 10825); |
| | ENST00000650581.1 (SEQ ID NO: 10827) |
| transcript | ENST00000469614.1 (SEQ ID NO: 10792); ENST00000477454.1 (SEQ ID NO: 10794); |
| | ENST00000477680.6 (SEQ ID NO: 10796); ENST00000490487.1 (SEQ ID NO: 10798); |
| | ENST00000491375.1 (SEQ ID NO: 10800); ENST00000496303.6 (SEQ ID NO: 10802); |
| | ENST00000647628.1 (SEQ ID NO: 10804); ENST00000647984.1 (SEQ ID NO: 10806); |
| | ENST00000648237.1 (SEQ ID NO: 10808); ENST00000648360.1 (SEQ ID NO: 10810); |
| | ENST00000648765.1 (SEQ ID NO: 10812); ENST00000648925.1 (SEQ ID NO: 10814); |
| | ENST00000649063.2 (SEQ ID NO: 10816); ENST00000649315.1 (SEQ ID NO: 10818); |
| | ENST00000649419.1 (SEQ ID NO: 10820); ENST00000649736.1 (SEQ ID NO: 10822); |
| | ENST00000650310.1 (SEQ ID NO: 10824); ENST00000650451.1 (SEQ ID NO: 10826); |
| | ENST00000650581.1 (SEQ ID NO: 10828) |

Target: ARNTL

| | |
|---|---|
| pre-mRNA | ENST00000389707.8 (SEQ ID NO: 15436); ENST00000401424.6 (SEQ ID NO: 15437); |
| transcript | ENST00000403290.5 (SEQ ID NO: 15438); ENST00000403482.7 (SEQ ID NO: 15439); |
| | ENST00000403510.8 (SEQ ID NO: 15440); ENST00000472842.1 (SEQ ID NO: 10834); |
| | ENST00000480685.5 (SEQ ID NO: 15441); ENST00000482049.6 (SEQ ID NO: 15442); |
| | ENST00000485918.2 (SEQ ID NO: 10838); ENST00000497429.5 (SEQ ID NO: 10840); |
| | ENST00000524392.5 (SEQ ID NO: 10842); ENST00000527998.5 (SEQ ID NO: 15443); |
| | ENST00000529050.5 (SEQ ID NO: 15444); ENST00000529388.6 (SEQ ID NO: 15445); |
| | ENST00000529390.1 (SEQ ID NO: 10847); ENST00000529825.6 (SEQ ID NO: 15446); |
| | ENST00000530357.6 (SEQ ID NO: 15447); ENST00000531665.5 (SEQ ID NO: 15448); |
| | ENST00000533520.5 (SEQ ID NO: 15449); ENST00000534102.1 (SEQ ID NO: 10853); |
| | ENST00000534544.5 (SEQ ID NO: 15450); ENST00000673626.1 (SEQ ID NO: 15451); |
| | ENST00000673817.1 (SEQ ID NO: 15452); ENST00000673834.1 (SEQ ID NO: 15453); |
| | ENST00000673837.1 (SEQ ID NO: 15454); ENST00000673868.1 (SEQ ID NO: 10860); |
| | ENST00000673888.1 (SEQ ID NO: 15455); ENST00000673892.1 (SEQ ID NO: 15456); |
| | ENST00000674108.1; (SEQ ID NO: 15457) |
| transcript | ENST00000389707.8 (SEQ ID NO: 10829); ENST00000401424.6 (SEQ ID NO: 10830); |
| | ENST00000403290.5 (SEQ ID NO: 10831); ENST00000403482.7 (SEQ ID NO: 10832); |
| | ENST00000403510.8 (SEQ ID NO: 10833); ENST00000472842.1 (SEQ ID NO: 10835); |
| | ENST00000480685.5 (SEQ ID NO: 10836); ENST00000482049.6 (SEQ ID NO: 10837); |
| | ENST00000485918.2 (SEQ ID NO: 10839); ENST00000497429.5 (SEQ ID NO: 10841); |
| | ENST00000524392.5 (SEQ ID NO: 10843); ENST00000527998.5 (SEQ ID NO: 10844); |
| | ENST00000529050.5 (SEQ ID NO: 10845); ENST00000529388.6 (SEQ ID NO: 10846); |
| | ENST00000529390.1 (SEQ ID NO: 10848); ENST00000529825.6 (SEQ ID NO: 10849); |
| | ENST00000530357.6 (SEQ ID NO: 10850); ENST00000531665.5 (SEQ ID NO: 10851); |
| | ENST00000533520.5 (SEQ ID NO: 10852); ENST00000534102.1 (SEQ ID NO: 10854); |
| | ENST00000534544.5 (SEQ ID NO: 10855); ENST00000673626.1 (SEQ ID NO: 10856); |
| | ENST00000673817.1 (SEQ ID NO: 10857); ENST00000673834.1 (SEQ ID NO: 10858); |
| | ENST00000673837.1 (SEQ ID NO: 10859); ENST00000673868.1 (SEQ ID NO: 10861); |
| | ENST00000673888.1 (SEQ ID NO: 10862); ENST00000673892.1 (SEQ ID NO: 10863); |
| | ENST00000674108.1 (SEQ ID NO: 10864) |

Target: ASAP3

| | |
|---|---|
| pre-mRNA | ENST00000336689.8 (SEQ ID NO: 15458); ENST00000437606.6 (SEQ ID NO: 15459); |
| transcript | ENST00000449467.2 (SEQ ID NO: 10867); ENST00000465372.5 (SEQ ID NO: 10869); |
| | ENST00000475814.5 (SEQ ID NO: 10871); ENST00000478858.5 (SEQ ID NO: 15460); |
| | ENST00000484418.1 (SEQ ID NO: 10874); ENST00000492982.6 (SEQ ID NO: 15461); |
| | ENST00000495646.5 (SEQ ID NO: 10877); ENST00000530874.1 (SEQ ID NO: 10879); |
| | ENST00000608765.1 (SEQ ID NO: 15462); ENST00000618240.4 (SEQ ID NO: 15463) |
| transcript | ENST00000336689.8 (SEQ ID NO: 10865); ENST00000437606.6 (SEQ ID NO: 10866); |
| | ENST00000449467.2 (SEQ ID NO: 10868); ENST00000465372.5 (SEQ ID NO: 10870); |
| | ENST00000475814.5 (SEQ ID NO: 10872); ENST00000478858.5 (SEQ ID NO: 10873); |
| | ENST00000484418.1 (SEQ ID NO: 10875); ENST00000492982.6 (SEQ ID NO: 10876); |
| | ENST00000495646.5 (SEQ ID NO: 10878); ENST00000530874.1 (SEQ ID NO: 10880); |
| | ENST00000608765.1 (SEQ ID NO: 10881); ENST00000618240.4 (SEQ ID NO: 10882) |

Target: ATRX

| | |
|---|---|
| pre-mRNA | ENST00000373344.10 (SEQ ID NO: 15464); ENST00000395603.7 (SEQ ID NO: 15465); |
| transcript | ENST00000400866.4 (SEQ ID NO: 10885); ENST00000460639.2 (SEQ ID NO: 10887); |
| | ENST00000479487.1 (SEQ ID NO: 10889); ENST00000480283.5 (SEQ ID NO: 15466); |
| | ENST00000493470.2 (SEQ ID NO: 10892); ENST00000622960.1 (SEQ ID NO: 15467); |
| | ENST00000623242.3 (SEQ ID NO: 10895); ENST00000623316.1 (SEQ ID NO: 10897); |
| | ENST00000623321.3 (SEQ ID NO: 15468); ENST00000623706.3 (SEQ ID NO: 15469); |
| | ENST00000624032.3 (SEQ ID NO: 15470); ENST00000624166.3 (SEQ ID NO: 15471); |
| | ENST00000624193.1 (SEQ ID NO: 15472); ENST00000624403.1 (SEQ ID NO: 10904); |
| | ENST00000624668.3 (SEQ ID NO: 15473); ENST00000624766.1 (SEQ ID NO: 10907); |
| | ENST00000625063.3 (SEQ ID NO: 10909); ENST00000635865.1 (SEQ ID NO: 10911); |
| | ENST00000636152.1 (SEQ ID NO: 10913); ENST00000636868.1 (SEQ ID NO: 10915); |
| | ENST00000637175.1 (SEQ ID NO: 10917); ENST00000637959.1 (SEQ ID NO: 10919) |
| transcript | ENST00000373344.10 (SEQ ID NO: 10883); ENST00000395603.7 (SEQ ID NO: 10884); |
| | ENST00000400866.4 (SEQ ID NO: 10886); ENST00000460639.2 (SEQ ID NO: 10888); |
| | ENST00000479487.1 (SEQ ID NO: 10890); ENST00000480283.5 (SEQ ID NO: 10891); |
| | ENST00000493470.2 (SEQ ID NO: 10893); ENST00000622960.1 (SEQ ID NO: 10894); |
| | ENST00000623242.3 (SEQ ID NO: 10896); ENST00000623316.1 (SEQ ID NO: 10898); |
| | ENST00000623321.3 (SEQ ID NO: 10899); ENST00000623706.3 (SEQ ID NO: 10900); |
| | ENST00000624032.3 (SEQ ID NO: 10901); ENST00000624166.3 (SEQ ID NO: 10902); |
| | ENST00000624193.1 (SEQ ID NO: 10903); ENST00000624403.1 (SEQ ID NO: 10905); |
| | ENST00000624668.3 (SEQ ID NO: 10906); ENST00000624766.1 (SEQ ID NO: 10908); |
| | ENST00000625063.3 (SEQ ID NO: 10910); ENST00000635865.1 (SEQ ID NO: 10912); |
| | ENST00000636152.1 (SEQ ID NO: 10914); ENST00000636868.1 (SEQ ID NO: 10916); |
| | ENST00000637175.1 (SEQ ID NO: 10918); ENST00000637959.1 (SEQ ID NO: 10920) |

Target: BBS2

| | |
|---|---|
| pre-mRNA | ENST00000245157.10 (SEQ ID NO: 15474); ENST00000561853.1 (SEQ ID NO: 10922); |
| transcript | ENST00000561877.1 (SEQ ID NO: 10924); ENST00000561951.5 (SEQ ID NO: 10926); |
| | ENST00000562012.1 (SEQ ID NO: 10928); ENST00000562059.1 (SEQ ID NO: 10930); |
| | ENST00000562813.1 (SEQ ID NO: 10932); ENST00000564123.6 (SEQ ID NO: 10934); |
| | ENST00000564459.5 (SEQ ID NO: 10936); ENST00000565378.1 (SEQ ID NO: 10938); |
| | ENST00000565781.5 (SEQ ID NO: 15475); ENST00000565859.1 (SEQ ID NO: 10941); |
| | ENST00000566210.1 (SEQ ID NO: 10943); ENST00000566410.1 (SEQ ID NO: 10945); |
| | ENST00000566452.1 (SEQ ID NO: 10947); ENST00000566495.1 (SEQ ID NO: 10949); |
| | ENST00000566689.5 (SEQ ID NO: 10951); ENST00000568104.5 (SEQ ID NO: 15476); |
| | ENST00000569192.5 (SEQ ID NO: 10954); ENST00000569342.5 (SEQ ID NO: 10956); |
| | ENST00000569941.5 (SEQ ID NO: 10958) |
| transcript | ENST00000245157.10 (SEQ ID NO: 10921); ENST00000561853.1 (SEQ ID NO: 10923); |
| | ENST00000561877.1 (SEQ ID NO: 10925); ENST00000561951.5 (SEQ ID NO: 10927); |
| | ENST00000562012.1 (SEQ ID NO: 10929); ENST00000562059.1 (SEQ ID NO: 10931); |
| | ENST00000562813.1 (SEQ ID NO: 10933); ENST00000564123.6 (SEQ ID NO: 10935); |
| | ENST00000564459.5 (SEQ ID NO: 10937); ENST00000565378.1 (SEQ ID NO: 10939); |
| | ENST00000565781.5 (SEQ ID NO: 10940); ENST00000565859.1 (SEQ ID NO: 10942); |
| | ENST00000566210.1 (SEQ ID NO: 10944); ENST00000566410.1 (SEQ ID NO: 10946); |
| | ENST00000566452.1 (SEQ ID NO: 10948); ENST00000566495.1 (SEQ ID NO: 10950); |
| | ENST00000566689.5 (SEQ ID NO: 10952); ENST00000568104.5 (SEQ ID NO: 10953); |
| | ENST00000569192.5 (SEQ ID NO: 10955); ENST00000569342.5 (SEQ ID NO: 10957); |
| | ENST00000569941.5 (SEQ ID NO: 10959) |

Target: BBS4

| | |
|---|---|
| pre-mRNA | ENST00000268057.9 (SEQ ID NO: 15477); ENST00000395205.6 (SEQ ID NO: 15478); |
| transcript | ENST00000561914.5 (SEQ ID NO: 15479); ENST00000562084.5 (SEQ ID NO: 15480); |
| | ENST00000562219.1 (SEQ ID NO: 10964); ENST00000563600.5 (SEQ ID NO: 15481); |
| | ENST00000564239.1 (SEQ ID NO: 15482); ENST00000565160.5 (SEQ ID NO: 15483); |
| | ENST00000566197.1 (SEQ ID NO: 10969); ENST00000566400.5 (SEQ ID NO: 15484); |
| | ENST00000566829.1 (SEQ ID NO: 15485); ENST00000566938.5 (SEQ ID NO: 15486); |
| | ENST00000567279.5 (SEQ ID NO: 15487); ENST00000568535.1 (SEQ ID NO: 10975); |
| | ENST00000569001.1 (SEQ ID NO: 10977); ENST00000569151.1 (SEQ ID NO: 10979); |
| | ENST00000569338.5 (SEQ ID NO: 15488); ENST00000569440.5 (SEQ ID NO: 15489) |
| transcript | ENST00000268057.9 (SEQ ID NO: 10960); ENST00000395205.6 (SEQ ID NO: 10961); |
| | ENST00000561914.5 (SEQ ID NO: 10962); ENST00000562084.5 (SEQ ID NO: 10963); |
| | ENST00000562219.1 (SEQ ID NO: 10965); ENST00000563600.5 (SEQ ID NO: 10966); |
| | ENST00000564239.1 (SEQ ID NO: 10967); ENST00000565160.5 (SEQ ID NO: 10968); |
| | ENST00000566197.1 (SEQ ID NO: 10970); ENST00000566400.5 (SEQ ID NO: 10971); |
| | ENST00000566829.1 (SEQ ID NO: 10972); ENST00000566938.5 (SEQ ID NO: 10973); |
| | ENST00000567279.5 (SEQ ID NO: 10974); ENST00000568535.1 (SEQ ID NO: 10976); |
| | ENST00000569001.1 (SEQ ID NO: 10978); ENST00000569151.1 (SEQ ID NO: 10980); |
| | ENST00000569338.5 (SEQ ID NO: 10981); ENST00000569440.5 (SEQ ID NO: 10982) |

Target: BRD9

| | |
|---|---|
| pre-mRNA | ENST00000466684.5 (SEQ ID NO: 10983); ENST00000467963.6 (SEQ ID NO: 10985); |
| transcript | ENST00000475706.5 (SEQ ID NO: 10987); ENST00000483173.5 (SEQ ID NO: 10989); |
| | ENST00000483234.5 (SEQ ID NO: 10991); ENST00000487688.1 (SEQ ID NO: 10993); |
| | ENST00000489093.1 (SEQ ID NO: 10995); ENST00000489816.5 (SEQ ID NO: 10997); |
| | ENST00000490814.6 (SEQ ID NO: 10999); ENST00000493082.5 (SEQ ID NO: 11001); |
| | ENST00000494422.1 (SEQ ID NO: 11003); ENST00000495265.5 (SEQ ID NO: 11005); |
| | ENST00000495794.5 (SEQ ID NO: 11007); ENST00000497410.5 (SEQ ID NO: 11009); |
| | ENST00000518250.5 (SEQ ID NO: 11011); ENST00000518251.1 (SEQ ID NO: 11013); |
| | ENST00000519112.5 (SEQ ID NO: 11015); ENST00000519838.5 (SEQ ID NO: 11017); |
| | ENST00000523139.5 (SEQ ID NO: 11019) |
| transcript | ENST00000466684.5 (SEQ ID NO: 10984); ENST00000467963.6 (SEQ ID NO: 10986); |
| | ENST00000475706.5 (SEQ ID NO: 10988); ENST00000483173.5 (SEQ ID NO: 10990); |
| | ENST00000483234.5 (SEQ ID NO: 10992); ENST00000487688.1 (SEQ ID NO: 10994); |
| | ENST00000489093.1 (SEQ ID NO: 10996); ENST00000489816.5 (SEQ ID NO: 10998); |
| | ENST00000490814.6 (SEQ ID NO: 11000); ENST00000493082.5 (SEQ ID NO: 11002); |
| | ENST00000494422.1 (SEQ ID NO: 11004); ENST00000495265.5 (SEQ ID NO: 11006); |
| | ENST00000495794.5 (SEQ ID NO: 11008); ENST00000497410.5 (SEQ ID NO: 11010); |
| | ENST00000518250.5 (SEQ ID NO: 11012); ENST00000518251.1 (SEQ ID NO: 11014); |
| | ENST00000519112.5 (SEQ ID NO: 11016); ENST00000519838.5 (SEQ ID NO: 11018); |
| | ENST00000523139.5 (SEQ ID NO: 11020) |

Target: CALM3

| | |
|---|---|
| pre-mRNA | ENST00000291295.14 (SEQ ID NO: 11021); ENST00000391918.6 (SEQ ID NO: 11023); |
| transcript | ENST00000477244.5 (SEQ ID NO: 11025); ENST00000482455.5 (SEQ ID NO: 11027); |
| | ENST00000486500.1 (SEQ ID NO: 11029); ENST00000594523.5 (SEQ ID NO: 11031); |
| | ENST00000595072.2 (SEQ ID NO: 11033); ENST00000596362.1 (SEQ ID NO: 11035); |
| | ENST00000597743.5 (SEQ ID NO: 11037); ENST00000597868.5 (SEQ ID NO: 11039); |
| | ENST00000598871.5 (SEQ ID NO: 11041); ENST00000599839.5 (SEQ ID NO: 11043); |
| | ENST00000602169.2 (SEQ ID NO: 11045) |
| transcript | ENST00000291295.14 (SEQ ID NO: 11022); ENST00000391918.6 (SEQ ID NO: 11024); |
| | ENST00000477244.5 (SEQ ID NO: 11026); ENST00000482455.5 (SEQ ID NO: 11028); |
| | ENST00000486500.1 (SEQ ID NO: 11030); ENST00000594523.5 (SEQ ID NO: 11032); |
| | ENST00000595072.2 (SEQ ID NO: 11034); ENST00000596362.1 (SEQ ID NO: 11036); |
| | ENST00000597743.5 (SEQ ID NO: 11038); ENST00000597868.5 (SEQ ID NO: 11040); |
| | ENST00000598871.5 (SEQ ID NO: 11042); ENST00000599839.5 (SEQ ID NO: 11044); |
| | ENST00000602169.2 (SEQ ID NO: 11046) |

Target: CARS1

| | |
|---|---|
| pre-mRNA | ENST00000278224.13 (SEQ ID NO: 15490); ENST00000380525.8 (SEQ ID NO: 15491); |
| transcript | ENST00000397111.9 (SEQ ID NO: 15492); ENST00000439280.6 (SEQ ID NO: 11050); |
| | ENST00000465207.5 (SEQ ID NO: 11052); ENST00000465240.1 (SEQ ID NO: 11054); |
| | ENST00000465331.1 (SEQ ID NO: 11056); ENST00000466442.6 (SEQ ID NO: 11058); |
| | ENST00000470221.6 (SEQ ID NO: 11060); ENST00000484484.5 (SEQ ID NO: 11062); |
| | ENST00000524825.5 (SEQ ID NO: 11064); ENST00000526890.5 (SEQ ID NO: 15493); |
| | ENST00000527330.2 (SEQ ID NO: 11067); ENST00000529772.5 (SEQ ID NO: 15494); |
| | ENST00000531387.5 (SEQ ID NO: 15495); ENST00000639317.1 (SEQ ID NO: 11071) |
| transcript | ENST00000278224.13 (SEQ ID NO: 11047); ENST00000380525.8 (SEQ ID NO: 11048); |
| | ENST00000397111.9 (SEQ ID NO: 11049); ENST00000439280.6 (SEQ ID NO: 11051); |
| | ENST00000465207.5 (SEQ ID NO: 11053); ENST00000465240.1 (SEQ ID NO: 11055); |
| | ENST00000465331.1 (SEQ ID NO: 11057); ENST00000466442.6 (SEQ ID NO: 11059); |
| | ENST00000470221.6 (SEQ ID NO: 11061); ENST00000484484.5 (SEQ ID NO: 11063); |
| | ENST00000524825.5 (SEQ ID NO: 11065); ENST00000526890.5 (SEQ ID NO: 11066); |
| | ENST00000527330.2 (SEQ ID NO: 11068); ENST00000529772.5 (SEQ ID NO: 11069); |
| | ENST00000531387.5 (SEQ ID NO: 11070); ENST00000639317.1 (SEQ ID NO: 11072) |

Target: CASP9

| | |
|---|---|
| pre-mRNA | ENST00000333868.10 (SEQ ID NO: 15496); ENST00000348549.9 (SEQ ID NO: 15497); |
| transcript | ENST00000375890.8 (SEQ ID NO: 15498); ENST00000400777.7 (SEQ ID NO: 15499); |
| | ENST00000424908.5 (SEQ ID NO: 11077); ENST00000440484.1 (SEQ ID NO: 11079); |
| | ENST00000447522.5 (SEQ ID NO: 11081); ENST00000469637.1 (SEQ ID NO: 11083); |
| | ENST00000474305.2 (SEQ ID NO: 15500); ENST00000546424.5 (SEQ ID NO: 15501); |
| | ENST00000546969.1 (SEQ ID NO: 11087) |
| transcript | ENST00000333868.10 (SEQ ID NO: 11073); ENST00000348549.9 (SEQ ID NO: 11074); |
| | ENST00000375890.8 (SEQ ID NO: 11075); ENST00000400777.7 (SEQ ID NO: 11076); |
| | ENST00000424908.5 (SEQ ID NO: 11078); ENST00000440484.1 (SEQ ID NO: 11080); |
| | ENST00000447522.5 (SEQ ID NO: 11082); ENST00000469637.1 (SEQ ID NO: 11084); |
| | ENST00000474305.2 (SEQ ID NO: 11085); ENST00000546424.5 (SEQ ID NO: 11086); |
| | ENST00000546969.1 (SEQ ID NO: 11088) |

Target: CATSPER1

| | |
|---|---|
| pre-mRNA | ENST00000312106.6 (SEQ ID NO: 11089); ENST00000529244.1 (SEQ ID NO: 11091) |
| transcript | |
| transcript | ENST00000312106.6 (SEQ ID NO: 11090); ENST00000529244.1 (SEQ ID NO: 11092) |

Target: CCDC40

| | |
|---|---|
| pre-mRNA | ENST00000269318.9 (SEQ ID NO: 15502); ENST00000374876.4 (SEQ ID NO: 15503); |
| transcript | ENST00000374877.7 (SEQ ID NO: 15504); ENST00000397545.9 (SEQ ID NO: 15505); |
| | ENST00000571028.1 (SEQ ID NO: 11097); ENST00000572083.5 (SEQ ID NO: 11099); |
| | ENST00000572253.5 (SEQ ID NO: 11101); ENST00000572270.1 (SEQ ID NO: 11103); |
| | ENST00000573474.5 (SEQ ID NO: 11105); ENST00000573903.1 (SEQ ID NO: 11107); |
| | ENST00000574099.1 (SEQ ID NO: 11109); ENST00000574799.5 (SEQ ID NO: 15506); |
| | ENST00000574933.1 (SEQ ID NO: 11112); ENST00000575431.1 (SEQ ID NO: 11114); |
| | ENST00000576033.5 (SEQ ID NO: 11116); ENST00000576241.1; (SEQ ID NO: 11118) |
| transcript | ENST00000269318.9 (SEQ ID NO: 11093); ENST00000374876.4 (SEQ ID NO: 11094); |
| | ENST00000374877.7 (SEQ ID NO: 11095); ENST00000397545.9 (SEQ ID NO: 11096); |
| | ENST00000571028.1 (SEQ ID NO: 11098); ENST00000572083.5 (SEQ ID NO: 11100); |
| | ENST00000572253.5 (SEQ ID NO: 11102); ENST00000572270.1 (SEQ ID NO: 11104); |
| | ENST00000573474.5 (SEQ ID NO: 11106); ENST00000573903.1 (SEQ ID NO: 11108); |
| | ENST00000574099.1 (SEQ ID NO: 11110); ENST00000574799.5 (SEQ ID NO: 11111); |
| | ENST00000574933.1 (SEQ ID NO: 11113); ENST00000575431.1 (SEQ ID NO: 11115); |
| | ENST00000576033.5 (SEQ ID NO: 11117); ENST00000576241.1 (SEQ ID NO: 11119) |

Target: CCDC88B

| | |
|---|---|
| pre-mRNA | ENST00000301897.5 (SEQ ID NO: 11120); ENST00000356786.10 (SEQ ID NO: 11122); |
| transcript | ENST00000359902.2 (SEQ ID NO: 11124); ENST00000463837.5 (SEQ ID NO: 11126); |
| | ENST00000472524.1 (SEQ ID NO: 11128); ENST00000473405.1 (SEQ ID NO: 11130); |
| | ENST00000479965.5 (SEQ ID NO: 11132); ENST00000492980.1 (SEQ ID NO: 11134); |
| | ENST00000494080.5 (SEQ ID NO: 11136); ENST00000494566.5 (SEQ ID NO: 11138) |
| transcript | ENST00000301897.5 (SEQ ID NO: 11121); ENST00000356786.10 (SEQ ID NO: 11123); |
| | ENST00000359902.2 (SEQ ID NO: 11125); ENST00000463837.5 (SEQ ID NO: 11127); |
| | ENST00000472524.1 (SEQ ID NO: 11129); ENST00000473405.1 (SEQ ID NO: 11131); |
| | ENST00000479965.5 (SEQ ID NO: 11133); ENST00000492980.1 (SEQ ID NO: 11135); |
| | ENST00000494080.5 (SEQ ID NO: 11137); ENST00000494566.5 (SEQ ID NO: 11139) |

Target: CD4

| | |
|---|---|
| pre-mRNA | ENST00000011653.9 (SEQ ID NO: 15507); ENST00000437800.6 (SEQ ID NO: 15508); |
| transcript | ENST00000535466.5 (SEQ ID NO: 11142); ENST00000535707.5 (SEQ ID NO: 11144); |
| | ENST00000536563.1 (SEQ ID NO: 11146); ENST00000536590.1 (SEQ ID NO: 11148); |
| | ENST00000536610.5 (SEQ ID NO: 11150); ENST00000538827.5 (SEQ ID NO: 11152); |
| | ENST00000539492.1 (SEQ ID NO: 11154); ENST00000541982.5 (SEQ ID NO: 11156); |
| | ENST00000543755.1 (SEQ ID NO: 11158); ENST00000544344.5 (SEQ ID NO: 11160) |
| transcript | ENST00000011653.9 (SEQ ID NO: 11140); ENST00000437800.6 (SEQ ID NO: 11141); |
| | ENST00000535466.5 (SEQ ID NO: 11143); ENST00000535707.5 (SEQ ID NO: 11145); |
| | ENST00000536563.1 (SEQ ID NO: 11147); ENST00000536590.1 (SEQ ID NO: 11149); |
| | ENST00000536610.5 (SEQ ID NO: 11151); ENST00000538827.5 (SEQ ID NO: 11153); |
| | ENST00000539492.1 (SEQ ID NO: 11155); ENST00000541982.5 (SEQ ID NO: 11157); |
| | ENST00000543755.1 (SEQ ID NO: 11159); ENST00000544344.5 (SEQ ID NO: 11161) |

Target: CD58

| | |
|---|---|
| pre-mRNA | ENST00000369487.3 (SEQ ID NO: 15509); ENST00000369489.10 (SEQ ID NO: 15510); |
| transcript | ENST00000457047.6 (SEQ ID NO: 15511); ENST00000464088.5 (SEQ ID NO: 15512); |
| | ENST00000526981.1 (SEQ ID NO: 11166) |
| transcript | ENST00000369487.3 (SEQ ID NO: 11162); ENST00000369489.10 (SEQ ID NO: 11163); |
| | ENST00000457047.6 (SEQ ID NO: 11164); ENST00000464088.5 (SEQ ID NO: 11165); |
| | ENST00000526981.1 (SEQ ID NO: 11167) |

Target: CES2

| | |
|---|---|
| pre-mRNA | ENST00000317091.10 (SEQ ID NO: 11168); ENST00000417689.6 (SEQ ID NO: 11170); |
| transcript | ENST00000561697.5 (SEQ ID NO: 11172); ENST00000561843.1 (SEQ ID NO: 11174); |
| | ENST00000563988.1 (SEQ ID NO: 11176); ENST00000564420.1 (SEQ ID NO: 11178); |
| | ENST00000566182.1 (SEQ ID NO: 11180); ENST00000566359.1 (SEQ ID NO: 11182); |
| | ENST00000567128.1 (SEQ ID NO: 11184); ENST00000568347.1 (SEQ ID NO: 11186); |
| | ENST00000568470.6 (SEQ ID NO: 11188); ENST00000570032.2 (SEQ ID NO: 11190); |
| | ENST00000652196.1 (SEQ ID NO: 11192); ENST00000652667.1; (SEQ ID NO: 11194) |
| transcript | ENST00000317091.10 (SEQ ID NO: 11169); ENST00000417689.6 (SEQ ID NO: 11171); |
| | ENST00000561697.5 (SEQ ID NO: 11173); ENST00000561843.1 (SEQ ID NO: 11175); |
| | ENST00000563988.1 (SEQ ID NO: 11177); ENST00000564420.1 (SEQ ID NO: 11179); |
| | ENST00000566182.1 (SEQ ID NO: 11181); ENST00000566359.1 (SEQ ID NO: 11183); |
| | ENST00000567128.1 (SEQ ID NO: 11185); ENST00000568347.1 (SEQ ID NO: 11187); |
| | ENST00000568470.6 (SEQ ID NO: 11189); ENST00000570032.2 (SEQ ID NO: 11191); |
| | ENST00000652196.1 (SEQ ID NO: 11193); ENST00000652667.1 (SEQ ID NO: 11195) |

Target: CHFR

| | |
|---|---|
| pre-mRNA | ENST00000266880.11 (SEQ ID NO: 15513); ENST00000315585.11 (SEQ ID NO: 15514); |
| transcript | ENST00000432561.6 (SEQ ID NO: 15515); ENST00000443047.6 (SEQ ID NO: 15516); |
| | ENST00000450056.6 (SEQ ID NO: 15517); ENST00000499045.2 (SEQ ID NO: 15518); |
| | ENST00000502279.2 (SEQ ID NO: 11202); ENST00000511001.2 (SEQ ID NO: 11204); |
| | ENST00000535181.5 (SEQ ID NO: 11206); ENST00000535527.5 (SEQ ID NO: 11208); |
| | ENST00000535897.1 (SEQ ID NO: 11210); ENST00000536196.1 (SEQ ID NO: 11212); |
| | ENST00000536843.5 (SEQ ID NO: 11214); ENST00000536932.5 (SEQ ID NO: 15519); |
| | ENST00000537551.5 (SEQ ID NO: 11217); ENST00000538235.2 (SEQ ID NO: 11219); |
| | ENST00000540537.5 (SEQ ID NO: 15520); ENST00000540963.1 (SEQ ID NO: 11222); |
| | ENST00000541341.5 (SEQ ID NO: 11224); ENST00000541817.5 (SEQ ID NO: 15521); |
| | ENST00000542714.5 (SEQ ID NO: 15522); ENST00000544093.5 (SEQ ID NO: 11228); |
| | ENST00000544268.5 (SEQ ID NO: 11230); ENST00000545046.5 (SEQ ID NO: 11232) |
| transcript | ENST00000266880.11 (SEQ ID NO: 11196); ENST00000315585.11 (SEQ ID NO: 11197); |
| | ENST00000432561.6 (SEQ ID NO: 11198); ENST00000443047.6 (SEQ ID NO: 11199); |
| | ENST00000450056.6 (SEQ ID NO: 11200); ENST00000499045.2 (SEQ ID NO: 11201); |
| | ENST00000502279.2 (SEQ ID NO: 11203); ENST00000511001.2 (SEQ ID NO: 11205); |
| | ENST00000535181.5 (SEQ ID NO: 11207); ENST00000535527.5 (SEQ ID NO: 11209); |
| | ENST00000535897.1 (SEQ ID NO: 11211); ENST00000536196.1 (SEQ ID NO: 11213); |
| | ENST00000536843.5 (SEQ ID NO: 11215); ENST00000536932.5 (SEQ ID NO: 11216); |
| | ENST00000537551.5 (SEQ ID NO: 11218); ENST00000538235.2 (SEQ ID NO: 11220); |
| | ENST00000540537.5 (SEQ ID NO: 11221); ENST00000540963.1 (SEQ ID NO: 11223); |
| | ENST00000541341.5 (SEQ ID NO: 11225); ENST00000541817.5 (SEQ ID NO: 11226); |
| | ENST00000542714.5 (SEQ ID NO: 11227); ENST00000544093.5 (SEQ ID NO: 11229); |
| | ENST00000544268.5 (SEQ ID NO: 11231); ENST00000545046.5 (SEQ ID NO: 11233) |

Target: CLCN2

| | |
|---|---|
| pre-mRNA | ENST00000265593.9 (SEQ ID NO: 11234); ENST00000344937.11 (SEQ ID NO: 11236); |
| transcript | ENST00000430397.5 (SEQ ID NO: 11238); ENST00000434054.6 (SEQ ID NO: 11240); |
| | ENST00000457512.1 (SEQ ID NO: 11242); ENST00000465231.1 (SEQ ID NO: 11244); |
| | ENST00000475279.2 (SEQ ID NO: 11246); ENST00000485667.1 (SEQ ID NO: 11248); |
| | ENST00000491162.1 (SEQ ID NO: 11250); ENST00000636180.1 (SEQ ID NO: 11252); |
| | ENST00000636241.1 (SEQ ID NO: 11254); ENST00000636419.1 (SEQ ID NO: 11256); |
| | ENST00000636492.1 (SEQ ID NO: 11258); ENST00000636658.1 (SEQ ID NO: 11260); |
| | ENST00000636661.1 (SEQ ID NO: 11262); ENST00000636830.1 (SEQ ID NO: 11264); |
| | ENST00000636860.1 (SEQ ID NO: 11266); ENST00000637258.1 (SEQ ID NO: 11268); |
| | ENST00000637392.1 (SEQ ID NO: 11270); ENST00000637538.1 (SEQ ID NO: 11272); |
| | ENST00000637909.1 (SEQ ID NO: 11274); ENST00000638134.1 (SEQ ID NO: 11276) |
| transcript | ENST00000265593.9 (SEQ ID NO: 11235); ENST00000344937.11 (SEQ ID NO: 11237); |
| | ENST00000430397.5 (SEQ ID NO: 11239); ENST00000434054.6 (SEQ ID NO: 11241); |
| | ENST00000457512.1 (SEQ ID NO: 11243); ENST00000465231.1 (SEQ ID NO: 11245); |
| | ENST00000475279.2 (SEQ ID NO: 11247); ENST00000485667.1 (SEQ ID NO: 11249); |
| | ENST00000491162.1 (SEQ ID NO: 11251); ENST00000636180.1 (SEQ ID NO: 11253); |
| | ENST00000636241.1 (SEQ ID NO: 11255); ENST00000636419.1 (SEQ ID NO: 11257); |
| | ENST00000636492.1 (SEQ ID NO: 11259); ENST00000636658.1 (SEQ ID NO: 11261); |
| | ENST00000636661.1 (SEQ ID NO: 11263); ENST00000636830.1 (SEQ ID NO: 11265); |
| | ENST00000636860.1 (SEQ ID NO: 11267); ENST00000637258.1 (SEQ ID NO: 11269); |
| | ENST00000637392.1 (SEQ ID NO: 11271); ENST00000637538.1 (SEQ ID NO: 11273); |
| | ENST00000637909.1 (SEQ ID NO: 11275); ENST00000638134.1 (SEQ ID NO: 11277) |

Target: CNOT3

| | |
|---|---|
| pre-mRNA | ENST00000221232.11 (SEQ ID NO: 11278); ENST00000358389.7 (SEQ ID NO: 11280); |
| transcript | ENST00000440571.6 (SEQ ID NO: 11282); ENST00000447684.5 (SEQ ID NO: 11284); |
| | ENST00000457463.1 (SEQ ID NO: 11286); ENST00000471126.1 (SEQ ID NO: 11288); |
| | ENST00000496327.2 (SEQ ID NO: 11290); ENST00000613073.4 (SEQ ID NO: 11292); |
| | ENST00000617930.2 (SEQ ID NO: 11294); ENST00000618939.5 (SEQ ID NO: 11296); |
| | ENST00000642159.1 (SEQ ID NO: 11298); ENST00000644245.1 (SEQ ID NO: 11300); |
| | ENST00000644707.1 (SEQ ID NO: 11302); ENST00000646002.1 (SEQ ID NO: 11304); |
| | ENST00000647082.1 (SEQ ID NO: 11306) |
| transcript | ENST00000221232.11 (SEQ ID NO: 11279); ENST00000358389.7 (SEQ ID NO: 11281); |
| | ENST00000440571.6 (SEQ ID NO: 11283); ENST00000447684.5 (SEQ ID NO: 11285); |
| | ENST00000457463.1 (SEQ ID NO: 11287); ENST00000471126.1 (SEQ ID NO: 11289); |
| | ENST00000496327.2 (SEQ ID NO: 11291); ENST00000613073.4 (SEQ ID NO: 11293); |
| | ENST00000617930.2 (SEQ ID NO: 11295); ENST00000618939.5 (SEQ ID NO: 11297); |
| | ENST00000642159.1 (SEQ ID NO: 11299); ENST00000644245.1 (SEQ ID NO: 11301); |
| | ENST00000644707.1 (SEQ ID NO: 11303); ENST00000646002.1 (SEQ ID NO: 11305); |
| | ENST00000647082.1 (SEQ ID NO: 11307) |

Target: CNTROB

| | |
|---|---|
| pre-mRNA | ENST00000380262.7 (SEQ ID NO: 11308); ENST00000563694.6 (SEQ ID NO: 11310); |
| transcript | ENST00000565740.5 (SEQ ID NO: 11312); ENST00000570782.1 (SEQ ID NO: 11314); |
| | ENST00000570784.1 (SEQ ID NO: 11316); ENST00000571540.5 (SEQ ID NO: 11318); |
| | ENST00000571632.5 (SEQ ID NO: 11320); ENST00000573862.1 (SEQ ID NO: 11322); |
| | ENST00000574430.5 (SEQ ID NO: 11324); ENST00000575408.5 (SEQ ID NO: 11326); |
| | ENST00000576536.1 (SEQ ID NO: 11328); ENST00000576538.5 (SEQ ID NO: 11330); |
| | ENST00000576587.1 (SEQ ID NO: 11332); ENST00000576723.5 (SEQ ID NO: 11334); |
| | ENST00000576922.5 (SEQ ID NO: 11336) |
| transcript | ENST00000380262.7 (SEQ ID NO: 11309); ENST00000563694.6 (SEQ ID NO: 11311); |
| | ENST00000565740.5 (SEQ ID NO: 11313); ENST00000570782.1 (SEQ ID NO: 11315); |
| | ENST00000570784.1 (SEQ ID NO: 11317); ENST00000571540.5 (SEQ ID NO: 11319); |
| | ENST00000571632.5 (SEQ ID NO: 11321); ENST00000573862.1 (SEQ ID NO: 11323); |
| | ENST00000574430.5 (SEQ ID NO: 11325); ENST00000575408.5 (SEQ ID NO: 11327); |
| | ENST00000576536.1 (SEQ ID NO: 11329); ENST00000576538.5 (SEQ ID NO: 11331); |
| | ENST00000576587.1 (SEQ ID NO: 11333); ENST00000576723.5 (SEQ ID NO: 11335); |
| | ENST00000576922.5 (SEQ ID NO: 11337) |

Target: COL5A3

| | |
|---|---|
| pre-mRNA | ENST00000264828.4 (SEQ ID NO: 15523); ENST00000461214.1; (SEQ ID NO: 11339) |
| transcript | |
| transcript | ENST00000264828.4 (SEQ ID NO: 11338); ENST00000461214.1 (SEQ ID NO: 11340) |

Target: CPSF1

| | |
|---|---|
| pre-mRNA | ENST00000526271.2 (SEQ ID NO: 11341); ENST00000527827.1 (SEQ ID NO: 11343); |
| transcript | ENST00000527916.1 (SEQ ID NO: 11345); ENST00000529288.1 (SEQ ID NO: 11347); |
| | ENST00000531042.5 (SEQ ID NO: 11349); ENST00000531480.2 (SEQ ID NO: 11351); |
| | ENST00000531727.5 (SEQ ID NO: 11353); ENST00000532560.5 (SEQ ID NO: 11355); |
| | ENST00000532725.1 (SEQ ID NO: 11357); ENST00000532935.5 (SEQ ID NO: 11359); |
| | ENST00000533492.1 (SEQ ID NO: 11361); ENST00000616140.2 (SEQ ID NO: 11363); |
| | ENST00000620219.4 (SEQ ID NO: 11365); ENST00000622776.1; (SEQ ID NO: 11367) |
| transcript | ENST00000526271.2 (SEQ ID NO: 11342); ENST00000527827.1 (SEQ ID NO: 11344); |
| | ENST00000527916.1 (SEQ ID NO: 11346); ENST00000529288.1 (SEQ ID NO: 11348); |
| | ENST00000531042.5 (SEQ ID NO: 11350); ENST00000531480.2 (SEQ ID NO: 11352); |
| | ENST00000531727.5 (SEQ ID NO: 11354); ENST00000532560.5 (SEQ ID NO: 11356); |
| | ENST00000532725.1 (SEQ ID NO: 11358); ENST00000532935.5 (SEQ ID NO: 11360); |
| | ENST00000533492.1 (SEQ ID NO: 11362); ENST00000616140.2 (SEQ ID NO: 11364); |
| | ENST00000620219.4 (SEQ ID NO: 11366); ENST00000622776.1 (SEQ ID NO: 11368) |

Target: CSPP1

| | |
|---|---|
| pre-mRNA | ENST00000262210.9 (SEQ ID NO: 15524); ENST00000519163.6 (SEQ ID NO: 15525); |
| transcript | ENST00000519668.1 (SEQ ID NO: 15526); ENST00000519701.5 (SEQ ID NO: 11372); |
| | ENST00000521168.5 (SEQ ID NO: 15527); ENST00000521324.1 (SEQ ID NO: 11375); |
| | ENST00000521919.5; (SEQ ID NO: 15528) |
| transcript | ENST00000262210.9 (SEQ ID NO: 11369); ENST00000519163.6 (SEQ ID NO: 11370); |
| | ENST00000519668.1 (SEQ ID NO: 11371); ENST00000519701.5 (SEQ ID NO: 11373); |
| | ENST00000521168.5 (SEQ ID NO: 11374); ENST00000521324.1 (SEQ ID NO: 11376); |
| | ENST00000521919.5 (SEQ ID NO: 11377) |

Target: CTF1

| | |
|---|---|
| pre-mRNA | ENST00000279804.2 (SEQ ID NO: 11378); ENST00000395019.3 (SEQ ID NO: 11380) |
| transcript | |
| transcript | ENST00000279804.2 (SEQ ID NO: 11379); ENST00000395019.3 (SEQ ID NO: 11381) |

Target: CTH

| | |
|---|---|
| pre-mRNA | ENST00000346806.2 (SEQ ID NO: 11382); ENST00000370938.8 (SEQ ID NO: 11384); |
| transcript | ENST00000411986.6 (SEQ ID NO: 11386); ENST00000464926.1 (SEQ ID NO: 11388); |
| | ENST00000482383.1 (SEQ ID NO: 11390) |
| transcript | ENST00000346806.2 (SEQ ID NO: 11383); ENST00000370938.8 (SEQ ID NO: 11385); |
| | ENST00000411986.6 (SEQ ID NO: 11387); ENST00000464926.1 (SEQ ID NO: 11389); |
| | ENST00000482383.1 (SEQ ID NO: 11391) |

Target: CYP3A5

| | |
|---|---|
| pre-mRNA | ENST00000222982.8 (SEQ ID NO: 15529); ENST00000339843.6 (SEQ ID NO: 15530); |
| transcript | ENST00000439761.3 (SEQ ID NO: 11394); ENST00000456417.5 (SEQ ID NO: 11396); |
| | ENST00000461920.5 (SEQ ID NO: 15531); ENST00000463364.5 (SEQ ID NO: 11399); |
| | ENST00000463907.5 (SEQ ID NO: 11401); ENST00000466061.5 (SEQ ID NO: 11403); |
| | ENST00000469622.5 (SEQ ID NO: 11405); ENST00000469887.5 (SEQ ID NO: 11407); |
| | ENST00000473347.1 (SEQ ID NO: 11409); ENST00000480723.5 (SEQ ID NO: 11411); |
| | ENST00000481825.5 (SEQ ID NO: 11413); ENST00000488187.1 (SEQ ID NO: 11415); |
| | ENST00000489231.1 (SEQ ID NO: 11417); ENST00000646887.1 (SEQ ID NO: 15532) |
| transcript | ENST00000222982.8 (SEQ ID NO: 11392); ENST00000339843.6 (SEQ ID NO: 11393); |
| | ENST00000439761.3 (SEQ ID NO: 11395); ENST00000456417.5 (SEQ ID NO: 11397); |
| | ENST00000461920.5 (SEQ ID NO: 11398); ENST00000463364.5 (SEQ ID NO: 11400); |
| | ENST00000463907.5 (SEQ ID NO: 11402); ENST00000466061.5 (SEQ ID NO: 11404); |
| | ENST00000469622.5 (SEQ ID NO: 11406); ENST00000469887.5 (SEQ ID NO: 11408); |
| | ENST00000473347.1 (SEQ ID NO: 11410); ENST00000480723.5 (SEQ ID NO: 11412); |
| | ENST00000481825.5 (SEQ ID NO: 11414); ENST00000488187.1 (SEQ ID NO: 11416); |
| | ENST00000489231.1 (SEQ ID NO: 11418); ENST00000646887.1 (SEQ ID NO: 11419) |

Target: DAB2IP

| | |
|---|---|
| pre-mRNA | ENST00000259371.6 (SEQ ID NO: 15533); ENST00000309989.1 (SEQ ID NO: 15534); |
| transcript | ENST00000373782.7 (SEQ ID NO: 15535); ENST00000394340.7 (SEQ ID NO: 15536); |
| | ENST00000408936.7 (SEQ ID NO: 15537); ENST00000436835.5 (SEQ ID NO: 15538); |
| | ENST00000459906.1 (SEQ ID NO: 11426); ENST00000465078.1 (SEQ ID NO: 11428); |
| | ENST00000487716.1 (SEQ ID NO: 11430); ENST00000489314.1 (SEQ ID NO: 15539); |
| | ENST00000648444.1 (SEQ ID NO: 15540); ENST00000648693.1 (SEQ ID NO: 11434) |
| transcript | ENST00000259371.6 (SEQ ID NO: 11420); ENST00000309989.1 (SEQ ID NO: 11421); |
| | ENST00000373782.7 (SEQ ID NO: 11422); ENST00000394340.7 (SEQ ID NO: 11423); |
| | ENST00000408936.7 (SEQ ID NO: 11424); ENST00000436835.5 (SEQ ID NO: 11425); |
| | ENST00000459906.1 (SEQ ID NO: 11427); ENST00000465078.1 (SEQ ID NO: 11429); |
| | ENST00000487716.1 (SEQ ID NO: 11431); ENST00000489314.1 (SEQ ID NO: 11432); |
| | ENST00000648444.1 (SEQ ID NO: 11433); ENST00000648693.1 (SEQ ID NO: 11435) |

Target: DBN1

| | |
|---|---|
| pre-mRNA | ENST00000292385.9 (SEQ ID NO: 11436); ENST00000309007.9 (SEQ ID NO: 11438); |
| transcript | ENST00000393565.5 (SEQ ID NO: 11440); ENST00000467054.2 (SEQ ID NO: 11442); |
| | ENST00000471767.1 (SEQ ID NO: 11444); ENST00000472831.5 (SEQ ID NO: 11446); |
| | ENST00000477391.6 (SEQ ID NO: 11448); ENST00000505550.1 (SEQ ID NO: 11450); |
| | ENST00000506117.5 (SEQ ID NO: 11452); ENST00000512501.1 (SEQ ID NO: 11454); |
| | ENST00000514833.1 (SEQ ID NO: 11456) |
| transcript | ENST00000292385.9 (SEQ ID NO: 11437); ENST00000309007.9 (SEQ ID NO: 11439); |
| | ENST00000393565.5 (SEQ ID NO: 11441); ENST00000467054.2 (SEQ ID NO: 11443); |
| | ENST00000471767.1 (SEQ ID NO: 11445); ENST00000472831.5 (SEQ ID NO: 11447); |
| | ENST00000477391.6 (SEQ ID NO: 11449); ENST00000505550.1 (SEQ ID NO: 11451); |
| | ENST00000506117.5 (SEQ ID NO: 11453); ENST00000512501.1 (SEQ ID NO: 11455); |
| | ENST00000514833.1 (SEQ ID NO: 11457) |

Target: DDX51

| | |
|---|---|
| pre-mRNA | ENST00000329073.8 (SEQ ID NO: 11458); ENST00000397333.4 (SEQ ID NO: 11460); |
| transcript | ENST00000462829.2 (SEQ ID NO: 11462); ENST00000541489.5 (SEQ ID NO: 11464); |
| | ENST00000545991.1 (SEQ ID NO: 11466); ENST00000546058.1 (SEQ ID NO: 11468) |
| transcript | ENST00000329073.8 (SEQ ID NO: 11459); ENST00000397333.4 (SEQ ID NO: 11461); |
| | ENST00000462829.2 (SEQ ID NO: 11463); ENST00000541489.5 (SEQ ID NO: 11465); |
| | ENST00000545991.1 (SEQ ID NO: 11467); ENST00000546058.1 (SEQ ID NO: 11469) |

Target: DEAF1

| | |
|---|---|
| pre-mRNA | ENST00000382409.3 (SEQ ID NO: 15541); ENST00000524786.1 (SEQ ID NO: 11471); |
| transcript | ENST00000525626.5 (SEQ ID NO: 11473); ENST00000525904.5 (SEQ ID NO: 11475); |
| | ENST00000526790.1 (SEQ ID NO: 11477); ENST00000526857.2 (SEQ ID NO: 11479); |
| | ENST00000527170.5 (SEQ ID NO: 15542); ENST00000527658.1 (SEQ ID NO: 11482); |
| | ENST00000528864.5 (SEQ ID NO: 11484); ENST00000529717.5 (SEQ ID NO: 11486); |
| | ENST00000529727.1 (SEQ ID NO: 11488); ENST00000530813.1 (SEQ ID NO: 15543) |
| transcript | ENST00000382409.3 (SEQ ID NO: 11470); ENST00000524786.1 (SEQ ID NO: 11472); |
| | ENST00000525626.5 (SEQ ID NO: 11474); ENST00000525904.5 (SEQ ID NO: 11476); |
| | ENST00000526790.1 (SEQ ID NO: 11478); ENST00000526857.2 (SEQ ID NO: 11480); |
| | ENST00000527170.5 (SEQ ID NO: 11481); ENST00000527658.1 (SEQ ID NO: 11483); |
| | ENST00000528864.5 (SEQ ID NO: 11485); ENST00000529717.5 (SEQ ID NO: 11487); |
| | ENST00000529727.1 (SEQ ID NO: 11489); ENST00000530813.1 (SEQ ID NO: 11490) |

Target: DGKE

| | |
|---|---|
| pre-mRNA | ENST00000284061.8 (SEQ ID NO: 15544); ENST00000570738.1 (SEQ ID NO: 11492); |
| transcript | ENST00000571084.1 (SEQ ID NO: 11494); ENST00000572810.1 (SEQ ID NO: 11496); |
| | ENST00000572944.1 (SEQ ID NO: 15545); ENST00000576869.5 (SEQ ID NO: 11499) |
| transcript | ENST00000284061.8 (SEQ ID NO: 11491); ENST00000570738.1 (SEQ ID NO: 11493); |
| | ENST00000571084.1 (SEQ ID NO: 11495); ENST00000572810.1 (SEQ ID NO: 11497); |
| | ENST00000572944.1 (SEQ ID NO: 11498); ENST00000576869.5 (SEQ ID NO: 11500) |

Target: DHDDS

| | |
|---|---|
| pre-mRNA | ENST00000236342.12 (SEQ ID NO: 15546); ENST00000360009.6 (SEQ ID NO: 15547); |
| transcript | ENST00000374185.7 (SEQ ID NO: 11503); ENST00000374186.7 (SEQ ID NO: 11505); |
| | ENST00000416052.1 (SEQ ID NO: 11507); ENST00000427245.6 (SEQ ID NO: 11509); |
| | ENST00000430232.5 (SEQ ID NO: 11511); ENST00000431933.5 (SEQ ID NO: 11513); |
| | ENST00000434391.6 (SEQ ID NO: 15548); ENST00000436153.6 (SEQ ID NO: 11516); |
| | ENST00000487944.5 (SEQ ID NO: 11518); ENST00000525165.5 (SEQ ID NO: 11520); |
| | ENST00000525326.5 (SEQ ID NO: 11522); ENST00000525410.1 (SEQ ID NO: 11524); |
| | ENST00000525546.5 (SEQ ID NO: 11526); ENST00000525682.6 (SEQ ID NO: 15549); |
| | ENST00000526219.5 (SEQ ID NO: 15550); ENST00000526278.5 (SEQ ID NO: 11530); |
| | ENST00000527611.1 (SEQ ID NO: 11532); ENST00000528557.5 (SEQ ID NO: 11534); |
| | ENST00000529688.5 (SEQ ID NO: 11536); ENST00000530781.5 (SEQ ID NO: 11538); |
| | ENST00000531312.5 (SEQ ID NO: 11540); ENST00000531955.5 (SEQ ID NO: 11542); |
| | ENST00000533087.5; (SEQ ID NO: 11544) |
| transcript | ENST00000236342.12 (SEQ ID NO: 11501); ENST00000360009.6 (SEQ ID NO: 11502); |
| | ENST00000374185.7 (SEQ ID NO: 11504); ENST00000374186.7 (SEQ ID NO: 11506); |
| | ENST00000416052.1 (SEQ ID NO: 11508); ENST00000427245.6 (SEQ ID NO: 11510); |
| | ENST00000430232.5 (SEQ ID NO: 11512); ENST00000431933.5 (SEQ ID NO: 11514); |
| | ENST00000434391.6 (SEQ ID NO: 11515); ENST00000436153.6 (SEQ ID NO: 11517); |
| | ENST00000487944.5 (SEQ ID NO: 11519); ENST00000525165.5 (SEQ ID NO: 11521); |
| | ENST00000525326.5 (SEQ ID NO: 11523); ENST00000525410.1 (SEQ ID NO: 11525); |
| | ENST00000525546.5 (SEQ ID NO: 11527); ENST00000525682.6 (SEQ ID NO: 11528); |
| | ENST00000526219.5 (SEQ ID NO: 11529); ENST00000526278.5 (SEQ ID NO: 11531); |
| | ENST00000527611.1 (SEQ ID NO: 11533); ENST00000528557.5 (SEQ ID NO: 11535); |
| | ENST00000529688.5 (SEQ ID NO: 11537); ENST00000530781.5 (SEQ ID NO: 11539); |
| | ENST00000531312.5 (SEQ ID NO: 11541); ENST00000531955.5 (SEQ ID NO: 11543); |
| | ENST00000533087.5 (SEQ ID NO: 11545) |

Target: DMKN

| | |
|---|---|
| pre-mRNA | ENST00000339686.8 (SEQ ID NO: 11546); ENST00000392206.6 (SEQ ID NO: 11548); |
| transcript | ENST00000402589.6 (SEQ ID NO: 11550); ENST00000408915.6 (SEQ ID NO: 11552); |
| | ENST00000414866.6 (SEQ ID NO: 11554); ENST00000418261.5 (SEQ ID NO: 11556); |
| | ENST00000419602.5 (SEQ ID NO: 11558); ENST00000424570.6 (SEQ ID NO: 11560); |
| | ENST00000429837.5 (SEQ ID NO: 11562); ENST00000434389.5 (SEQ ID NO: 11564); |
| | ENST00000436012.5 (SEQ ID NO: 11566); ENST00000443640.5 (SEQ ID NO: 11568); |
| | ENST00000447113.6 (SEQ ID NO: 11570); ENST00000450261.1 (SEQ ID NO: 11572); |
| | ENST00000451297.6 (SEQ ID NO: 11574); ENST00000458071.5 (SEQ ID NO: 11576); |
| | ENST00000460051.5 (SEQ ID NO: 11578); ENST00000460327.5 (SEQ ID NO: 11580); |
| | ENST00000461300.5 (SEQ ID NO: 11582); ENST00000462126.5 (SEQ ID NO: 11584); |
| | ENST00000462538.5 (SEQ ID NO: 11586); ENST00000462721.5 (SEQ ID NO: 11588); |
| | ENST00000463292.5 (SEQ ID NO: 11590); ENST00000464709.6 (SEQ ID NO: 11592); |
| | ENST00000464894.5 (SEQ ID NO: 11594); ENST00000465927.5 (SEQ ID NO: 11596); |
| | ENST00000466587.6 (SEQ ID NO: 11598); ENST00000467532.5 (SEQ ID NO: 11600); |
| | ENST00000467637.5 (SEQ ID NO: 11602); ENST00000469960.5 (SEQ ID NO: 11604); |
| | ENST00000470746.6 (SEQ ID NO: 11606); ENST00000470857.1 (SEQ ID NO: 11608); |
| | ENST00000470964.1 (SEQ ID NO: 11610); ENST00000471017.5 (SEQ ID NO: 11612); |
| | ENST00000471786.6 (SEQ ID NO: 11614); ENST00000472065.1 (SEQ ID NO: 11616); |
| | ENST00000472252.6 (SEQ ID NO: 11618); ENST00000472365.6 (SEQ ID NO: 11620); |
| | ENST00000474928.5 (SEQ ID NO: 11622); ENST00000474992.5 (SEQ ID NO: 11624); |
| | ENST00000476051.5 (SEQ ID NO: 11626); ENST00000476246.5 (SEQ ID NO: 11628); |
| | ENST00000478648.6 (SEQ ID NO: 11630); ENST00000480502.5 (SEQ ID NO: 11632); |
| | ENST00000480507.1 (SEQ ID NO: 11634); ENST00000482321.5 (SEQ ID NO: 11636); |
| | ENST00000483855.5 (SEQ ID NO: 11638); ENST00000484064.5 (SEQ ID NO: 11640); |
| | ENST00000486450.5 (SEQ ID NO: 11642); ENST00000486756.6 (SEQ ID NO: 11644); |
| | ENST00000488542.6 (SEQ ID NO: 11646); ENST00000488762.5 (SEQ ID NO: 11648); |
| | ENST00000488892.5 (SEQ ID NO: 11650); ENST00000489395.6 (SEQ ID NO: 11652); |
| | ENST00000490622.5 (SEQ ID NO: 11654); ENST00000492341.6 (SEQ ID NO: 11656); |
| | ENST00000493517.5 (SEQ ID NO: 11658); ENST00000493979.5 (SEQ ID NO: 11660); |
| | ENST00000498211.5 (SEQ ID NO: 11662); ENST00000498269.5 (SEQ ID NO: 11664); |
| | ENST00000498593.5 (SEQ ID NO: 11666); ENST00000593342.5 (SEQ ID NO: 11668); |
| | ENST00000595571.5 (SEQ ID NO: 11670); ENST00000597212.5 (SEQ ID NO: 11672); |
| | ENST00000601368.5 (SEQ ID NO: 11674); ENST00000601650.1 (SEQ ID NO: 11676); |
| | ENST00000602679.5 (SEQ ID NO: 11678); ENST00000602781.5 (SEQ ID NO: 11680) |
| transcript | ENST00000339686.8 (SEQ ID NO: 11547); ENST00000392206.6 (SEQ ID NO: 11549); |
| | ENST00000402589.6 (SEQ ID NO: 11551); ENST00000408915.6 (SEQ ID NO: 11553); |
| | ENST00000414866.6 (SEQ ID NO: 11555); ENST00000418261.5 (SEQ ID NO: 11557); |
| | ENST00000419602.5 (SEQ ID NO: 11559); ENST00000424570.6 (SEQ ID NO: 11561); |
| | ENST00000429837.5 (SEQ ID NO: 11563); ENST00000434389.5 (SEQ ID NO: 11565); |
| | ENST00000436012.5 (SEQ ID NO: 11567); ENST00000443640.5 (SEQ ID NO: 11569); |
| | ENST00000447113.6 (SEQ ID NO: 11571); ENST00000450261.1 (SEQ ID NO: 11573); |
| | ENST00000451297.6 (SEQ ID NO: 11575); ENST00000458071.5 (SEQ ID NO: 11577); |
| | ENST00000460051.5 (SEQ ID NO: 11579); ENST00000460327.5 (SEQ ID NO: 11581); |
| | ENST00000461300.5 (SEQ ID NO: 11583); ENST00000462126.5 (SEQ ID NO: 11585); |
| | ENST00000462538.5 (SEQ ID NO: 11587); ENST00000462721.5 (SEQ ID NO: 11589); |
| | ENST00000463292.5 (SEQ ID NO: 11591); ENST00000464709.6 (SEQ ID NO: 11593); |
| | ENST00000464894.5 (SEQ ID NO: 11595); ENST00000465927.5 (SEQ ID NO: 11597); |
| | ENST00000466587.6 (SEQ ID NO: 11599); ENST00000467532.5 (SEQ ID NO: 11601); |
| | ENST00000467637.5 (SEQ ID NO: 11603); ENST00000469960.5 (SEQ ID NO: 11605); |
| | ENST00000470746.6 (SEQ ID NO: 11607); ENST00000470857.1 (SEQ ID NO: 11609); |
| | ENST00000470964.1 (SEQ ID NO: 11611); ENST00000471017.5 (SEQ ID NO: 11613); |
| | ENST00000471786.6 (SEQ ID NO: 11615); ENST00000472065.1 (SEQ ID NO: 11617); |
| | ENST00000472252.6 (SEQ ID NO: 11619); ENST00000472365.6 (SEQ ID NO: 11621); |
| | ENST00000474928.5 (SEQ ID NO: 11623); ENST00000474992.5 (SEQ ID NO: 11625); |
| | ENST00000476051.5 (SEQ ID NO: 11627); ENST00000476246.5 (SEQ ID NO: 11629); |
| | ENST00000478648.6 (SEQ ID NO: 11631); ENST00000480502.5 (SEQ ID NO: 11633); |
| | ENST00000480507.1 (SEQ ID NO: 11635); ENST00000482321.5 (SEQ ID NO: 11637); |
| | ENST00000483855.5 (SEQ ID NO: 11639); ENST00000484064.5 (SEQ ID NO: 11641); |
| | ENST00000486450.5 (SEQ ID NO: 11643); ENST00000486756.6 (SEQ ID NO: 11645); |
| | ENST00000488542.6 (SEQ ID NO: 11647); ENST00000488762.5 (SEQ ID NO: 11649); |
| | ENST00000488892.5 (SEQ ID NO: 11651); ENST00000489395.6 (SEQ ID NO: 11653); |
| | ENST00000490622.5 (SEQ ID NO: 11655); ENST00000492341.6 (SEQ ID NO: 11657); |
| | ENST00000493517.5 (SEQ ID NO: 11659); ENST00000493979.5 (SEQ ID NO: 11661); |
| | ENST00000498211.5 (SEQ ID NO: 11663); ENST00000498269.5 (SEQ ID NO: 11665); |
| | ENST00000498593.5 (SEQ ID NO: 11667); ENST00000593342.5 (SEQ ID NO: 11669); |
| | ENST00000595571.5 (SEQ ID NO: 11671); ENST00000597212.5 (SEQ ID NO: 11673); |
| | ENST00000601368.5 (SEQ ID NO: 11675); ENST00000601650.1 (SEQ ID NO: 11677); |
| | ENST00000602679.5 (SEQ ID NO: 11679); ENST00000602781.5 (SEQ ID NO: 11681) |

Target: DNHD1

| | |
|---|---|
| pre-mRNA | ENST00000254579.11 (SEQ ID NO: 15551); ENST00000354685.7 (SEQ ID NO: 11683); |
| transcript | ENST00000472080.1 (SEQ ID NO: 11685); ENST00000473019.5 (SEQ ID NO: 11687); |
| | ENST00000477562.1 (SEQ ID NO: 11689); ENST00000524401.2 (SEQ ID NO: 11691); |
| | ENST00000525080.1 (SEQ ID NO: 11693); ENST00000525883.5 (SEQ ID NO: 11695); |
| | ENST00000526027.1 (SEQ ID NO: 11697); ENST00000527143.1 (SEQ ID NO: 11699); |
| | ENST00000529821.5 (SEQ ID NO: 11701); ENST00000530197.5 (SEQ ID NO: 11703); |
| | ENST00000531903.5 (SEQ ID NO: 11705); ENST00000532027.5 (SEQ ID NO: 11707); |
| | ENST00000532467.1 (SEQ ID NO: 11709); ENST00000533635.5 (SEQ ID NO: 11711); |
| | ENST00000533649.1 (SEQ ID NO: 11713); ENST00000534210.6 (SEQ ID NO: 15552) |
| transcript | ENST00000254579.11 (SEQ ID NO: 11682); ENST00000354685.7 (SEQ ID NO: 11684); |
| | ENST00000472080.1 (SEQ ID NO: 11686); ENST00000473019.5 (SEQ ID NO: 11688); |
| | ENST00000477562.1 (SEQ ID NO: 11690); ENST00000524401.2 (SEQ ID NO: 11692); |
| | ENST00000525080.1 (SEQ ID NO: 11694); ENST00000525883.5 (SEQ ID NO: 11696); |
| | ENST00000526027.1 (SEQ ID NO: 11698); ENST00000527143.1 (SEQ ID NO: 11700); |
| | ENST00000529821.5 (SEQ ID NO: 11702); ENST00000530197.5 (SEQ ID NO: 11704); |
| | ENST00000531903.5 (SEQ ID NO: 11706); ENST00000532027.5 (SEQ ID NO: 11708); |
| | ENST00000532467.1 (SEQ ID NO: 11710); ENST00000533635.5 (SEQ ID NO: 11712); |
| | ENST00000533649.1 (SEQ ID NO: 11714); ENST00000534210.6 (SEQ ID NO: 11715) |

Target: DOCK8

| | |
|---|---|
| pre-mRNA | ENST00000382329.1 (SEQ ID NO: 15553); ENST00000382331.5 (SEQ ID NO: 15554); |
| transcript | ENST00000382341.5 (SEQ ID NO: 15555); ENST00000432829.6 (SEQ ID NO: 15556); |
| | ENST00000453981.5 (SEQ ID NO: 15557); ENST00000454469.6 (SEQ ID NO: 15558); |
| | ENST00000462618.1 (SEQ ID NO: 11722); ENST00000469197.5 (SEQ ID NO: 15559); |
| | ENST00000469391.5 (SEQ ID NO: 15560); ENST00000474772.1 (SEQ ID NO: 11726); |
| | ENST00000478380.5 (SEQ ID NO: 15561); ENST00000479404.5 (SEQ ID NO: 15562); |
| | ENST00000483757.5 (SEQ ID NO: 15563); ENST00000487230.5 (SEQ ID NO: 15564); |
| | ENST00000493666.2 (SEQ ID NO: 11732); ENST00000495184.5 (SEQ ID NO: 15565); |
| | ENST00000524396.5 (SEQ ID NO: 15566) |
| transcript | ENST00000382329.1 (SEQ ID NO: 11716); ENST00000382331.5 (SEQ ID NO: 11717); |
| | ENST00000382341.5 (SEQ ID NO: 11718); ENST00000432829.6 (SEQ ID NO: 11719); |
| | ENST00000453981.5 (SEQ ID NO: 11720); ENST00000454469.6 (SEQ ID NO: 11721); |
| | ENST00000462618.1 (SEQ ID NO: 11723); ENST00000469197.5 (SEQ ID NO: 11724); |
| | ENST00000469391.5 (SEQ ID NO: 11725); ENST00000474772.1 (SEQ ID NO: 11727); |
| | ENST00000478380.5 (SEQ ID NO: 11728); ENST00000479404.5 (SEQ ID NO: 11729); |
| | ENST00000483757.5 (SEQ ID NO: 11730); ENST00000487230.5 (SEQ ID NO: 11731); |
| | ENST00000493666.2 (SEQ ID NO: 11733); ENST00000495184.5 (SEQ ID NO: 11734); |
| | ENST00000524396.5 (SEQ ID NO: 11735) |

Target: DOK5

| | |
|---|---|
| pre-mRNA | ENST00000262593.10 (SEQ ID NO: 15567); ENST00000395939.5 (SEQ ID NO: 15568); |
| transcript | ENST00000484860.1 (SEQ ID NO: 11738); ENST00000491469.1 (SEQ ID NO: 11740) |
| transcript | ENST00000262593.10 (SEQ ID NO: 11736); ENST00000395939.5 (SEQ ID NO: 11737); |
| | ENST00000484860.1 (SEQ ID NO: 11739); ENST00000491469.1 (SEQ ID NO: 11741) |

Target: EIF2B5

| | |
|---|---|
| pre-mRNA | ENST00000432569.2 (SEQ ID NO: 11742); ENST00000432982.5 (SEQ ID NO: 11744); |
| transcript | ENST00000465218.3 (SEQ ID NO: 11746); ENST00000468748.7 (SEQ ID NO: 11748); |
| | ENST00000471832.2 (SEQ ID NO: 11750); ENST00000479250.1 (SEQ ID NO: 11752); |
| | ENST00000481054.5 (SEQ ID NO: 11754); ENST00000484154.2 (SEQ ID NO: 11756); |
| | ENST00000491008.6 (SEQ ID NO: 11758); ENST00000491144.5 (SEQ ID NO: 11760); |
| | ENST00000492226.2 (SEQ ID NO: 11762); ENST00000492773.6 (SEQ ID NO: 11764); |
| | ENST00000493740.1 (SEQ ID NO: 11766); ENST00000498831.1 (SEQ ID NO: 11768); |
| | ENST00000647636.1 (SEQ ID NO: 11770); ENST00000647909.1 (SEQ ID NO: 11772); |
| | ENST00000648145.1 (SEQ ID NO: 11774); ENST00000648189.1 (SEQ ID NO: 11776); |
| | ENST00000648256.1 (SEQ ID NO: 11778); ENST00000648314.1 (SEQ ID NO: 11780); |
| | ENST00000648599.1 (SEQ ID NO: 11782); ENST00000648630.1 (SEQ ID NO: 11784); |
| | ENST00000648682.1 (SEQ ID NO: 11786); ENST00000648882.1 (SEQ ID NO: 11788); |
| | ENST00000648890.1 (SEQ ID NO: 11790); ENST00000648915.2 (SEQ ID NO: 11792); |
| | ENST00000649545.1 (SEQ ID NO: 11794); ENST00000649688.1 (SEQ ID NO: 11796); |
| | ENST00000649814.1 (SEQ ID NO: 11798); ENST00000650244.1 (SEQ ID NO: 11800); |
| | ENST00000650270.1 (SEQ ID NO: 11802) |
| transcript | ENST00000432569.2 (SEQ ID NO: 11743); ENST00000432982.5 (SEQ ID NO: 11745); |
| | ENST00000465218.3 (SEQ ID NO: 11747); ENST00000468748.7 (SEQ ID NO: 11749); |
| | ENST00000471832.2 (SEQ ID NO: 11751); ENST00000479250.1 (SEQ ID NO: 11753); |
| | ENST00000481054.5 (SEQ ID NO: 11755); ENST00000484154.2 (SEQ ID NO: 11757); |
| | ENST00000491008.6 (SEQ ID NO: 11759); ENST00000491144.5 (SEQ ID NO: 11761); |
| | ENST00000492226.2 (SEQ ID NO: 11763); ENST00000492773.6 (SEQ ID NO: 11765); |
| | ENST00000493740.1 (SEQ ID NO: 11767); ENST00000498831.1 (SEQ ID NO: 11769); |
| | ENST00000647636.1 (SEQ ID NO: 11771); ENST00000647909.1 (SEQ ID NO: 11773); |
| | ENST00000648145.1 (SEQ ID NO: 11775); ENST00000648189.1 (SEQ ID NO: 11777); |
| | ENST00000648256.1 (SEQ ID NO: 11779); ENST00000648314.1 (SEQ ID NO: 11781); |
| | ENST00000648599.1 (SEQ ID NO: 11783); ENST00000648630.1 (SEQ ID NO: 11785); |
| | ENST00000648682.1 (SEQ ID NO: 11787); ENST00000648882.1 (SEQ ID NO: 11789); |
| | ENST00000648890.1 (SEQ ID NO: 11791); ENST00000648915.2 (SEQ ID NO: 11793); |
| | ENST00000649545.1 (SEQ ID NO: 11795); ENST00000649688.1 (SEQ ID NO: 11797); |
| | ENST00000649814.1 (SEQ ID NO: 11799); ENST00000650244.1 (SEQ ID NO: 11801); |
| | ENST00000650270.1 (SEQ ID NO: 11803) |

Target: ELAC2

| | |
|---|---|
| pre-mRNA | ENST00000338034.9 (SEQ ID NO: 11804); ENST00000395962.6 (SEQ ID NO: 11806); |
| transcript | ENST00000426905.7 (SEQ ID NO: 11808); ENST00000446899.5 (SEQ ID NO: 11810); |
| | ENST00000465825.5 (SEQ ID NO: 11812); ENST00000476042.1 (SEQ ID NO: 11814); |
| | ENST00000480891.5 (SEQ ID NO: 11816); ENST00000484122.5 (SEQ ID NO: 11818); |
| | ENST00000487229.6 (SEQ ID NO: 11820); ENST00000491478.5 (SEQ ID NO: 11822); |
| | ENST00000492559.5 (SEQ ID NO: 11824); ENST00000578071.1 (SEQ ID NO: 11826); |
| | ENST00000578104.1 (SEQ ID NO: 11828); ENST00000578991.5 (SEQ ID NO: 11830); |
| | ENST00000580504.5 (SEQ ID NO: 11832); ENST00000581499.6 (SEQ ID NO: 11834); |
| | ENST00000583371.5 (SEQ ID NO: 11836); ENST00000584650.5 (SEQ ID NO: 11838); |
| | ENST00000609101.5 (SEQ ID NO: 11840); ENST00000609345.1 (SEQ ID NO: 11842); |
| | ENST00000609757.5 (SEQ ID NO: 11844) |
| transcript | ENST00000338034.9 (SEQ ID NO: 11805); ENST00000395962.6 (SEQ ID NO: 11807); |
| | ENST00000426905.7 (SEQ ID NO: 11809); ENST00000446899.5 (SEQ ID NO: 11811); |
| | ENST00000465825.5 (SEQ ID NO: 11813); ENST00000476042.1 (SEQ ID NO: 11815); |
| | ENST00000480891.5 (SEQ ID NO: 11817); ENST00000484122.5 (SEQ ID NO: 11819); |
| | ENST00000487229.6 (SEQ ID NO: 11821); ENST00000491478.5 (SEQ ID NO: 11823); |
| | ENST00000492559.5 (SEQ ID NO: 11825); ENST00000578071.1 (SEQ ID NO: 11827); |
| | ENST00000578104.1 (SEQ ID NO: 11829); ENST00000578991.5 (SEQ ID NO: 11831); |
| | ENST00000580504.5 (SEQ ID NO: 11833); ENST00000581499.6 (SEQ ID NO: 11835); |
| | ENST00000583371.5 (SEQ ID NO: 11837); ENST00000584650.5 (SEQ ID NO: 11839); |
| | ENST00000609101.5 (SEQ ID NO: 11841); ENST00000609345.1 (SEQ ID NO: 11843); |
| | ENST00000609757.5 (SEQ ID NO: 11845) |

Target: ELP1

| | |
|---|---|
| pre-mRNA | ENST00000374647.10 (SEQ ID NO: 15569); ENST00000467959.1 (SEQ ID NO: 11847); |
| transcript | ENST00000495759.5 (SEQ ID NO: 11849); ENST00000537196.1 (SEQ ID NO: 15570) |
| transcript | ENST00000374647.10 (SEQ ID NO: 11846); ENST00000467959.1 (SEQ ID NO: 11848); |
| | ENST00000495759.5 (SEQ ID NO: 11850); ENST00000537196.1 (SEQ ID NO: 11851) |

Target: ENTPD4

| | |
|---|---|
| pre-mRNA | ENST00000356206.10 (SEQ ID NO: 15571); ENST00000358689.9 (SEQ ID NO: 11853); |
| transcript | ENST00000417069.6 (SEQ ID NO: 11855); ENST00000518471.5 (SEQ ID NO: 15572); |
| | ENST00000518718.1 (SEQ ID NO: 11858); ENST00000519839.1 (SEQ ID NO: 11860); |
| | ENST00000520936.1 (SEQ ID NO: 11862); ENST00000521321.5 (SEQ ID NO: 15573); |
| | ENST00000522255.1 (SEQ ID NO: 11865); ENST00000522913.1 (SEQ ID NO: 11867); |
| | ENST00000523958.5 (SEQ ID NO: 11869) |
| transcript | ENST00000356206.10 (SEQ ID NO: 11852); ENST00000358689.9 (SEQ ID NO: 11854); |
| | ENST00000417069.6 (SEQ ID NO: 11856); ENST00000518471.5 (SEQ ID NO: 11857); |
| | ENST00000518718.1 (SEQ ID NO: 11859); ENST00000519839.1 (SEQ ID NO: 11861); |
| | ENST00000520936.1 (SEQ ID NO: 11863); ENST00000521321.5 (SEQ ID NO: 11864); |
| | ENST00000522255.1 (SEQ ID NO: 11866); ENST00000522913.1 (SEQ ID NO: 11868); |
| | ENST00000523958.5 (SEQ ID NO: 11870) |

Target: ESRRA

| | |
|---|---|
| pre-mRNA | ENST00000000442.11 (SEQ ID NO: 11871); ENST00000405666.5 (SEQ ID NO: 11873); |
| transcript | ENST00000406310.6 (SEQ ID NO: 11875); ENST00000467987.1 (SEQ ID NO: 11877); |
| | ENST00000468670.1 (SEQ ID NO: 11879); ENST00000539594.5 (SEQ ID NO: 11881); |
| | ENST00000545035.1 (SEQ ID NO: 11883); ENST00000673656.1 (SEQ ID NO: 11885) |
| transcript | ENST00000000442.11 (SEQ ID NO: 11872); ENST00000405666.5 (SEQ ID NO: 11874); |
| | ENST00000406310.6 (SEQ ID NO: 11876); ENST00000467987.1 (SEQ ID NO: 11878); |
| | ENST00000468670.1 (SEQ ID NO: 11880); ENST00000539594.5 (SEQ ID NO: 11882); |
| | ENST00000545035.1 (SEQ ID NO: 11884); ENST00000673656.1 (SEQ ID NO: 11886) |

Target: ESS2

| | |
|---|---|
| pre-mRNA | ENST00000252137.11 (SEQ ID NO: 11887); ENST00000434568.5 (SEQ ID NO: 11889); |
| transcript | ENST00000469466.1 (SEQ ID NO: 11891); ENST00000472073.1 (SEQ ID NO: 11893) |
| transcript | ENST00000252137.11 (SEQ ID NO: 11888); ENST00000434568.5 (SEQ ID NO: 11890); |
| | ENST00000469466.1 (SEQ ID NO: 11892); ENST00000472073.1 (SEQ ID NO: 11894) |

Target: ETAA1

| | |
|---|---|
| pre-mRNA | ENST00000272342.6 (SEQ ID NO: 11895); ENST00000462772.2 (SEQ ID NO: 11897); |
| transcript | ENST00000644028.1 (SEQ ID NO: 11899); ENST00000645739.1 (SEQ ID NO: 11901) |
| transcript | ENST00000272342.6 (SEQ ID NO: 11896); ENST00000462772.2 (SEQ ID NO: 11898); |
| | ENST00000644028.1 (SEQ ID NO: 11900); ENST00000645739.1 (SEQ ID NO: 11902) |

Target: ETHE1

| | |
|---|---|
| pre-mRNA | ENST00000292147.7 (SEQ ID NO: 11903); ENST00000594342.5 (SEQ ID NO: 11905); |
| transcript | ENST00000595115.1 (SEQ ID NO: 11907); ENST00000598330.1 (SEQ ID NO: 11909); |
| | ENST00000600651.5 (SEQ ID NO: 11911); ENST00000602138.1 (SEQ ID NO: 11913) |
| transcript | ENST00000292147.7 (SEQ ID NO: 11904); ENST00000594342.5 (SEQ ID NO: 11906); |
| | ENST00000595115.1 (SEQ ID NO: 11908); ENST00000598330.1 (SEQ ID NO: 11910); |
| | ENST00000600651.5 (SEQ ID NO: 11912); ENST00000602138.1 (SEQ ID NO: 11914) |

Target: FANCA

| | |
|---|---|
| pre-mRNA | ENST00000305699.15 (SEQ ID NO: 11915); ENST00000389301.8 (SEQ ID NO: 15574); |
| transcript | ENST00000389302.7 (SEQ ID NO: 11918); ENST00000534992.5 (SEQ ID NO: 11920); |
| | ENST00000543736.5 (SEQ ID NO: 11922); ENST00000561660.1 (SEQ ID NO: 11924); |
| | ENST00000561667.1 (SEQ ID NO: 11926); ENST00000561722.5 (SEQ ID NO: 11928); |
| | ENST00000562424.1 (SEQ ID NO: 11930); ENST00000563318.1 (SEQ ID NO: 11932); |
| | ENST00000563510.5 (SEQ ID NO: 11934); ENST00000563513.1 (SEQ ID NO: 11936); |
| | ENST00000563673.5 (SEQ ID NO: 11938); ENST00000563767.1 (SEQ ID NO: 11940); |
| | ENST00000564475.5 (SEQ ID NO: 11942); ENST00000564870.1 (SEQ ID NO: 11944); |
| | ENST00000564969.5 (SEQ ID NO: 11946); ENST00000565582.5 (SEQ ID NO: 11948); |
| | ENST00000566133.1 (SEQ ID NO: 11950); ENST00000566409.1 (SEQ ID NO: 11952); |
| | ENST00000566889.5 (SEQ ID NO: 11954); ENST00000567205.1 (SEQ ID NO: 11956); |
| | ENST00000567284.6 (SEQ ID NO: 11958); ENST00000567510.1 (SEQ ID NO: 11960); |
| | ENST00000567621.5 (SEQ ID NO: 11962); ENST00000567879.5 (SEQ ID NO: 11964); |
| | ENST00000567883.5 (SEQ ID NO: 11966); ENST00000567943.1 (SEQ ID NO: 11968); |
| | ENST00000567988.5 (SEQ ID NO: 11970); ENST00000568369.5 (SEQ ID NO: 15575); |
| | ENST00000568626.1 (SEQ ID NO: 11973); ENST00000568983.5 (SEQ ID NO: 11975) |
| transcript | ENST00000305699.15 (SEQ ID NO: 11916); ENST00000389301.8 (SEQ ID NO: 11917); |
| | ENST00000389302.7 (SEQ ID NO: 11919); ENST00000534992.5 (SEQ ID NO: 11921); |
| | ENST00000543736.5 (SEQ ID NO: 11923); ENST00000561660.1 (SEQ ID NO: 11925); |
| | ENST00000561667.1 (SEQ ID NO: 11927); ENST00000561722.5 (SEQ ID NO: 11929); |
| | ENST00000562424.1 (SEQ ID NO: 11931); ENST00000563318.1 (SEQ ID NO: 11933); |
| | ENST00000563510.5 (SEQ ID NO: 11935); ENST00000563513.1 (SEQ ID NO: 11937); |
| | ENST00000563673.5 (SEQ ID NO: 11939); ENST00000563767.1 (SEQ ID NO: 11941); |
| | ENST00000564475.5 (SEQ ID NO: 11943); ENST00000564870.1 (SEQ ID NO: 11945); |
| | ENST00000564969.5 (SEQ ID NO: 11947); ENST00000565582.5 (SEQ ID NO: 11949); |
| | ENST00000566133.1 (SEQ ID NO: 11951); ENST00000566409.1 (SEQ ID NO: 11953); |
| | ENST00000566889.5 (SEQ ID NO: 11955); ENST00000567205.1 (SEQ ID NO: 11957); |
| | ENST00000567284.6 (SEQ ID NO: 11959); ENST00000567510.1 (SEQ ID NO: 11961); |
| | ENST00000567621.5 (SEQ ID NO: 11963); ENST00000567879.5 (SEQ ID NO: 11965); |
| | ENST00000567883.5 (SEQ ID NO: 11967); ENST00000567943.1 (SEQ ID NO: 11969); |
| | ENST00000567988.5 (SEQ ID NO: 11971); ENST00000568369.5 (SEQ ID NO: 11972); |
| | ENST00000568626.1 (SEQ ID NO: 11974); ENST00000568983.5 (SEQ ID NO: 11976) |

Target: FASTK

| | |
|---|---|
| pre-mRNA | ENST00000297532.11 (SEQ ID NO: 11977); ENST00000353841.6 (SEQ ID NO: 11979); |
| transcript | ENST00000459800.1 (SEQ ID NO: 11981); ENST00000460980.5 (SEQ ID NO: 11983); |
| | ENST00000461979.1 (SEQ ID NO: 11985); ENST00000465272.1 (SEQ ID NO: 11987); |
| | ENST00000466855.1 (SEQ ID NO: 11989); ENST00000467237.5 (SEQ ID NO: 11991); |
| | ENST00000469237.5 (SEQ ID NO: 11993); ENST00000478477.1 (SEQ ID NO: 11995); |
| | ENST00000478883.1 (SEQ ID NO: 11997); ENST00000482571.2 (SEQ ID NO: 11999); |
| | ENST00000482806.5 (SEQ ID NO: 12001); ENST00000483953.5 (SEQ ID NO: 12003); |
| | ENST00000489884.5 (SEQ ID NO: 12005); ENST00000496663.1 (SEQ ID NO: 12007); |
| | ENST00000540185.5; (SEQ ID NO: 12009) |
| transcript | ENST00000297532.11 (SEQ ID NO: 11978); ENST00000353841.6 (SEQ ID NO: 11980); |
| | ENST00000459800.1 (SEQ ID NO: 11982); ENST00000460980.5 (SEQ ID NO: 11984); |
| | ENST00000461979.1 (SEQ ID NO: 11986); ENST00000465272.1 (SEQ ID NO: 11988); |
| | ENST00000466855.1 (SEQ ID NO: 11990); ENST00000467237.5 (SEQ ID NO: 11992); |
| | ENST00000469237.5 (SEQ ID NO: 11994); ENST00000478477.1 (SEQ ID NO: 11996); |
| | ENST00000478883.1 (SEQ ID NO: 11998); ENST00000482571.2 (SEQ ID NO: 12000); |
| | ENST00000482806.5 (SEQ ID NO: 12002); ENST00000483953.5 (SEQ ID NO: 12004); |
| | ENST00000489884.5 (SEQ ID NO: 12006); ENST00000496663.1 (SEQ ID NO: 12008); |
| | ENST00000540185.5 (SEQ ID NO: 12010) |

Target: FASTKD3

| | |
|---|---|
| pre-mRNA | ENST00000264669.10 (SEQ ID NO: 12011); ENST00000282110.8 (SEQ ID NO: 12013); |
| transcript | ENST00000504695.1 (SEQ ID NO: 12015); ENST00000507036.1 (SEQ ID NO: 12017); |
| | ENST00000507572.1 (SEQ ID NO: 12019); ENST00000511261.5 (SEQ ID NO: 12021); |
| | ENST00000513577.1 (SEQ ID NO: 12023); ENST00000513658.5 (SEQ ID NO: 12025) |
| transcript | ENST00000264669.10 (SEQ ID NO: 12012); ENST00000282110.8 (SEQ ID NO: 12014); |
| | ENST00000504695.1 (SEQ ID NO: 12016); ENST00000507036.1 (SEQ ID NO: 12018); |
| | ENST00000507572.1 (SEQ ID NO: 12020); ENST00000511261.5 (SEQ ID NO: 12022); |
| | ENST00000513577.1 (SEQ ID NO: 12024); ENST00000513658.5 (SEQ ID NO: 12026) |

Target: FPGS

| | |
|---|---|
| pre-mRNA | ENST00000373225.7 (SEQ ID NO: 12027); ENST00000373228.5 (SEQ ID NO: 12029); |
| transcript | ENST00000373247.7 (SEQ ID NO: 12031); ENST00000393706.6 (SEQ ID NO: 12033); |
| | ENST00000423577.1 (SEQ ID NO: 12035); ENST00000431857.5 (SEQ ID NO: 12037); |
| | ENST00000460181.5 (SEQ ID NO: 12039); ENST00000467826.5 (SEQ ID NO: 12041); |
| | ENST00000469310.1 (SEQ ID NO: 12043); ENST00000473536.1 (SEQ ID NO: 12045); |
| | ENST00000475270.1 (SEQ ID NO: 12047); ENST00000475765.5 (SEQ ID NO: 12049); |
| | ENST00000479147.6 (SEQ ID NO: 12051); ENST00000479375.6 (SEQ ID NO: 12053); |
| | ENST00000481552.6 (SEQ ID NO: 12055); ENST00000488307.5 (SEQ ID NO: 12057); |
| | ENST00000488506.5 (SEQ ID NO: 12059); ENST00000489522.5 (SEQ ID NO: 12061); |
| | ENST00000496586.5 (SEQ ID NO: 12063); ENST00000497386.5 (SEQ ID NO: 12065); |
| | ENST00000630236.2 (SEQ ID NO: 12067) |
| transcript | ENST00000373225.7 (SEQ ID NO: 12028); ENST00000373228.5 (SEQ ID NO: 12030); |
| | ENST00000373247.7 (SEQ ID NO: 12032); ENST00000393706.6 (SEQ ID NO: 12034); |
| | ENST00000423577.1 (SEQ ID NO: 12036); ENST00000431857.5 (SEQ ID NO: 12038); |
| | ENST00000460181.5 (SEQ ID NO: 12040); ENST00000467826.5 (SEQ ID NO: 12042); |
| | ENST00000469310.1 (SEQ ID NO: 12044); ENST00000473536.1 (SEQ ID NO: 12046); |
| | ENST00000475270.1 (SEQ ID NO: 12048); ENST00000475765.5 (SEQ ID NO: 12050); |
| | ENST00000479147.6 (SEQ ID NO: 12052); ENST00000479375.6 (SEQ ID NO: 12054); |
| | ENST00000481552.6 (SEQ ID NO: 12056); ENST00000488307.5 (SEQ ID NO: 12058); |
| | ENST00000488506.5 (SEQ ID NO: 12060); ENST00000489522.5 (SEQ ID NO: 12062); |
| | ENST00000496586.5 (SEQ ID NO: 12064); ENST00000497386.5 (SEQ ID NO: 12066); |
| | ENST00000630236.2 (SEQ ID NO: 12068) |

Target: FREM1

| | |
|---|---|
| pre-mRNA | ENST00000380875.7 (SEQ ID NO: 15576); ENST00000380880.3 (SEQ ID NO: 15577); |
| transcript | ENST00000380894.5 (SEQ ID NO: 15578); ENST00000422223.6 (SEQ ID NO: 15579); |
| | ENST00000427623.5 (SEQ ID NO: 15580); ENST00000466679.1 (SEQ ID NO: 12074); |
| | ENST00000485068.5 (SEQ ID NO: 12076); ENST00000486223.1 (SEQ ID NO: 12078); |
| | ENST00000497634.2; (SEQ ID NO: 12080) |
| transcript | ENST00000380875.7 (SEQ ID NO: 12069); ENST00000380880.3 (SEQ ID NO: 12070); |
| | ENST00000380894.5 (SEQ ID NO: 12071); ENST00000422223.6 (SEQ ID NO: 12072); |
| | ENST00000427623.5 (SEQ ID NO: 12073); ENST00000466679.1 (SEQ ID NO: 12075); |
| | ENST00000485068.5 (SEQ ID NO: 12077); ENST00000486223.1 (SEQ ID NO: 12079); |
| | ENST00000497634.2 (SEQ ID NO: 12081) |

Target: GAS8

| | |
|---|---|
| pre-mRNA | ENST00000268699.9 (SEQ ID NO: 12082); ENST00000536122.7 (SEQ ID NO: 12084); |
| transcript | ENST00000537797.5 (SEQ ID NO: 12086); ENST00000540721.5 (SEQ ID NO: 12088); |
| | ENST00000561675.1 (SEQ ID NO: 12090); ENST00000563980.5 (SEQ ID NO: 12092); |
| | ENST00000564392.5 (SEQ ID NO: 12094); ENST00000564789.5 (SEQ ID NO: 12096); |
| | ENST00000564802.1 (SEQ ID NO: 12098); ENST00000564853.1 (SEQ ID NO: 12100); |
| | ENST00000565000.1 (SEQ ID NO: 12102); ENST00000565062.5 (SEQ ID NO: 12104); |
| | ENST00000565957.5 (SEQ ID NO: 12106); ENST00000566266.5 (SEQ ID NO: 12108); |
| | ENST00000568284.1 (SEQ ID NO: 12110); ENST00000568664.5 (SEQ ID NO: 12112); |
| | ENST00000568705.5 (SEQ ID NO: 12114); ENST00000569399.1 (SEQ ID NO: 12116); |
| | ENST00000569558.5 (SEQ ID NO: 12118); ENST00000620723.4 (SEQ ID NO: 12120) |
| transcript | ENST00000268699.9 (SEQ ID NO: 12083); ENST00000536122.7 (SEQ ID NO: 12085); |
| | ENST00000537797.5 (SEQ ID NO: 12087); ENST00000540721.5 (SEQ ID NO: 12089); |
| | ENST00000561675.1 (SEQ ID NO: 12091); ENST00000563980.5 (SEQ ID NO: 12093); |
| | ENST00000564392.5 (SEQ ID NO: 12095); ENST00000564789.5 (SEQ ID NO: 12097); |
| | ENST00000564802.1 (SEQ ID NO: 12099); ENST00000564853.1 (SEQ ID NO: 12101); |
| | ENST00000565000.1 (SEQ ID NO: 12103); ENST00000565062.5 (SEQ ID NO: 12105); |
| | ENST00000565957.5 (SEQ ID NO: 12107); ENST00000566266.5 (SEQ ID NO: 12109); |
| | ENST00000568284.1 (SEQ ID NO: 12111); ENST00000568664.5 (SEQ ID NO: 12113); |
| | ENST00000568705.5 (SEQ ID NO: 12115); ENST00000569399.1 (SEQ ID NO: 12117); |
| | ENST00000569558.5 (SEQ ID NO: 12119); ENST00000620723.4 (SEQ ID NO: 12121) |

Target: GCK

| | |
|---|---|
| pre-mRNA | ENST00000336642.9 (SEQ ID NO: 12122); ENST00000345378.7 (SEQ ID NO: 12124); |
| transcript | ENST00000395796.7 (SEQ ID NO: 12126); ENST00000403799.8 (SEQ ID NO: 15581); |
| | ENST00000437084.1 (SEQ ID NO: 15582); ENST00000459642.1 (SEQ ID NO: 12130); |
| | ENST00000473353.1 (SEQ ID NO: 12132); ENST00000476008.1 (SEQ ID NO: 15583); |
| | ENST00000616242.4 (SEQ ID NO: 12135); ENST00000671824.1 (SEQ ID NO: 15584); |
| | ENST00000672743.1 (SEQ ID NO: 12138); ENST00000673284.1 (SEQ ID NO: 15585) |
| transcript | ENST00000336642.9 (SEQ ID NO: 12123); ENST00000345378.7 (SEQ ID NO: 12125); |
| | ENST00000395796.7 (SEQ ID NO: 12127); ENST00000403799.8 (SEQ ID NO: 12128); |
| | ENST00000437084.1 (SEQ ID NO: 12129); ENST00000459642.1 (SEQ ID NO: 12131); |
| | ENST00000473353.1 (SEQ ID NO: 12133); ENST00000476008.1 (SEQ ID NO: 12134); |
| | ENST00000616242.4 (SEQ ID NO: 12136); ENST00000671824.1 (SEQ ID NO: 12137); |
| | ENST00000672743.1 (SEQ ID NO: 12139); ENST00000673284.1 (SEQ ID NO: 12140) |

Target: GGA1

| | |
|---|---|
| pre-mRNA | ENST00000325180.12 (SEQ ID NO: 12141); ENST00000326597.6 (SEQ ID NO: 12143); |
| transcript | ENST00000343632.9 (SEQ ID NO: 12145); ENST00000381756.9 (SEQ ID NO: 12147); |
| | ENST00000405147.7 (SEQ ID NO: 12149); ENST00000406772.5 (SEQ ID NO: 12151); |
| | ENST00000411501.5 (SEQ ID NO: 12153); ENST00000413251.5 (SEQ ID NO: 12155); |
| | ENST00000423024.1 (SEQ ID NO: 12157); ENST00000429218.5 (SEQ ID NO: 12159); |
| | ENST00000431745.5 (SEQ ID NO: 12161); ENST00000439161.5 (SEQ ID NO: 12163); |
| | ENST00000447515.5 (SEQ ID NO: 12165); ENST00000449944.5 (SEQ ID NO: 12167); |
| | ENST00000453208.5 (SEQ ID NO: 12169); ENST00000460957.1 (SEQ ID NO: 12171); |
| | ENST00000463672.1 (SEQ ID NO: 12173); ENST00000475445.1 (SEQ ID NO: 12175); |
| | ENST00000481613.1 (SEQ ID NO: 12177); ENST00000484804.5 (SEQ ID NO: 12179); |
| | ENST00000488672.1 (SEQ ID NO: 12181); ENST00000489772.5 (SEQ ID NO: 12183); |
| | ENST00000491295.1; (SEQ ID NO: 12185) |
| transcript | ENST00000325180.12 (SEQ ID NO: 12142); ENST00000326597.6 (SEQ ID NO: 12144); |
| | ENST00000343632.9 (SEQ ID NO: 12146); ENST00000381756.9 (SEQ ID NO: 12148); |
| | ENST00000405147.7 (SEQ ID NO: 12150); ENST00000406772.5 (SEQ ID NO: 12152); |
| | ENST00000411501.5 (SEQ ID NO: 12154); ENST00000413251.5 (SEQ ID NO: 12156); |
| | ENST00000423024.1 (SEQ ID NO: 12158); ENST00000429218.5 (SEQ ID NO: 12160); |
| | ENST00000431745.5 (SEQ ID NO: 12162); ENST00000439161.5 (SEQ ID NO: 12164); |
| | ENST00000447515.5 (SEQ ID NO: 12166); ENST00000449944.5 (SEQ ID NO: 12168); |
| | ENST00000453208.5 (SEQ ID NO: 12170); ENST00000460957.1 (SEQ ID NO: 12172); |
| | ENST00000463672.1 (SEQ ID NO: 12174); ENST00000475445.1 (SEQ ID NO: 12176); |
| | ENST00000481613.1 (SEQ ID NO: 12178); ENST00000484804.5 (SEQ ID NO: 12180); |
| | ENST00000488672.1 (SEQ ID NO: 12182); ENST00000489772.5 (SEQ ID NO: 12184); |
| | ENST00000491295.1 (SEQ ID NO: 12186) |

Target: GGA3

| | |
|---|---|
| pre-mRNA | ENST00000537686.5 (SEQ ID NO: 12187); ENST00000538886.5 (SEQ ID NO: 12189); |
| transcript | ENST00000577435.5 (SEQ ID NO: 12191); ENST00000578208.1 (SEQ ID NO: 12193); |
| | ENST00000578348.5 (SEQ ID NO: 12195); ENST00000578773.1 (SEQ ID NO: 12197); |
| | ENST00000578896.1 (SEQ ID NO: 12199); ENST00000579743.2 (SEQ ID NO: 12201); |
| | ENST00000580646.5 (SEQ ID NO: 12203); ENST00000580799.2 (SEQ ID NO: 12205); |
| | ENST00000582232.1 (SEQ ID NO: 12207); ENST00000582376.1 (SEQ ID NO: 12209); |
| | ENST00000582486.5 (SEQ ID NO: 12211); ENST00000582717.5 (SEQ ID NO: 12213); |
| | ENST00000582821.5 (SEQ ID NO: 12215); ENST00000583282.5 (SEQ ID NO: 12217); |
| | ENST00000583667.1 (SEQ ID NO: 12219); ENST00000584243.5 (SEQ ID NO: 12221); |
| | ENST00000584550.1 (SEQ ID NO: 12223); ENST00000613421.4 (SEQ ID NO: 12225); |
| | ENST00000614198.4 (SEQ ID NO: 12227); ENST00000621217.4 (SEQ ID NO: 12229); |
| | ENST00000621870.4 (SEQ ID NO: 12231); ENST00000649398.1 (SEQ ID NO: 12233) |
| transcript | ENST00000537686.5 (SEQ ID NO: 12188); ENST00000538886.5 (SEQ ID NO: 12190); |
| | ENST00000577435.5 (SEQ ID NO: 12192); ENST00000578208.1 (SEQ ID NO: 12194); |
| | ENST00000578348.5 (SEQ ID NO: 12196); ENST00000578773.1 (SEQ ID NO: 12198); |
| | ENST00000578896.1 (SEQ ID NO: 12200); ENST00000579743.2 (SEQ ID NO: 12202); |
| | ENST00000580646.5 (SEQ ID NO: 12204); ENST00000580799.2 (SEQ ID NO: 12206); |
| | ENST00000582232.1 (SEQ ID NO: 12208); ENST00000582376.1 (SEQ ID NO: 12210); |
| | ENST00000582486.5 (SEQ ID NO: 12212); ENST00000582717.5 (SEQ ID NO: 12214); |
| | ENST00000582821.5 (SEQ ID NO: 12216); ENST00000583282.5 (SEQ ID NO: 12218); |
| | ENST00000583667.1 (SEQ ID NO: 12220); ENST00000584243.5 (SEQ ID NO: 12222); |
| | ENST00000584550.1 (SEQ ID NO: 12224); ENST00000613421.4 (SEQ ID NO: 12226); |
| | ENST00000614198.4 (SEQ ID NO: 12228); ENST00000621217.4 (SEQ ID NO: 12230); |
| | ENST00000621870.4 (SEQ ID NO: 12232); ENST00000649398.1 (SEQ ID NO: 12234) |

Target: GGT1

| | |
|---|---|
| pre-mRNA | ENST00000248923.9 (SEQ ID NO: 15586); ENST00000400380.5 (SEQ ID NO: 12236); |
| transcript | ENST00000400382.6 (SEQ ID NO: 12238); ENST00000401885.5 (SEQ ID NO: 12240); |
| | ENST00000403838.5 (SEQ ID NO: 12242); ENST00000404223.5 (SEQ ID NO: 12244); |
| | ENST00000404532.5 (SEQ ID NO: 12246); ENST00000404603.5 (SEQ ID NO: 15587); |
| | ENST00000404920.1 (SEQ ID NO: 12249); ENST00000411974.5 (SEQ ID NO: 12251); |
| | ENST00000412658.5 (SEQ ID NO: 12253); ENST00000412898.5 (SEQ ID NO: 12255); |
| | ENST00000419133.5 (SEQ ID NO: 12257); ENST00000425895.5 (SEQ ID NO: 12259); |
| | ENST00000428855.5 (SEQ ID NO: 12261); ENST00000430289.5 (SEQ ID NO: 12263); |
| | ENST00000432867.5 (SEQ ID NO: 12265); ENST00000438643.6 (SEQ ID NO: 12267); |
| | ENST00000439775.1 (SEQ ID NO: 15588); ENST00000445029.5 (SEQ ID NO: 12270); |
| | ENST00000447416.5 (SEQ ID NO: 12272); ENST00000451366.5 (SEQ ID NO: 12274); |
| | ENST00000452551.5 (SEQ ID NO: 12276); ENST00000455483.5 (SEQ ID NO: 12278); |
| | ENST00000456869.5 (SEQ ID NO: 12280); ENST00000466310.5 (SEQ ID NO: 12282); |
| | ENST00000474191.6 (SEQ ID NO: 12284); ENST00000487766.1 (SEQ ID NO: 12286); |
| | ENST00000490087.1 (SEQ ID NO: 12288); ENST00000651180.1 (SEQ ID NO: 15589); |
| | ENST00000652248.1 (SEQ ID NO: 15590) |
| transcript | ENST00000248923.9 (SEQ ID NO: 12235); ENST00000400380.5 (SEQ ID NO: 12237); |
| | ENST00000400382.6 (SEQ ID NO: 12239); ENST00000401885.5 (SEQ ID NO: 12241); |
| | ENST00000403838.5 (SEQ ID NO: 12243); ENST00000404223.5 (SEQ ID NO: 12245); |
| | ENST00000404532.5 (SEQ ID NO: 12247); ENST00000404603.5 (SEQ ID NO: 12248); |
| | ENST00000404920.1 (SEQ ID NO: 12250); ENST00000411974.5 (SEQ ID NO: 12252); |
| | ENST00000412658.5 (SEQ ID NO: 12254); ENST00000412898.5 (SEQ ID NO: 12256); |
| | ENST00000419133.5 (SEQ ID NO: 12258); ENST00000425895.5 (SEQ ID NO: 12260); |
| | ENST00000428855.5 (SEQ ID NO: 12262); ENST00000430289.5 (SEQ ID NO: 12264); |
| | ENST00000432867.5 (SEQ ID NO: 12266); ENST00000438643.6 (SEQ ID NO: 12268); |
| | ENST00000439775.1 (SEQ ID NO: 12269); ENST00000445029.5 (SEQ ID NO: 12271); |
| | ENST00000447416.5 (SEQ ID NO: 12273); ENST00000451366.5 (SEQ ID NO: 12275); |
| | ENST00000452551.5 (SEQ ID NO: 12277); ENST00000455483.5 (SEQ ID NO: 12279); |
| | ENST00000456869.5 (SEQ ID NO: 12281); ENST00000466310.5 (SEQ ID NO: 12283); |
| | ENST00000474191.6 (SEQ ID NO: 12285); ENST00000487766.1 (SEQ ID NO: 12287); |
| | ENST00000490087.1 (SEQ ID NO: 12289); ENST00000651180.1 (SEQ ID NO: 12290); |
| | ENST00000652248.1 (SEQ ID NO: 12291) |

Target: GLMN

| | |
|---|---|
| pre-mRNA | ENST00000370360.8 (SEQ ID NO: 15591); ENST00000463560.1 (SEQ ID NO: 12293); |
| transcript | ENST00000471465.1 (SEQ ID NO: 12295); ENST00000487911.1 (SEQ ID NO: 12297); |
| | ENST00000495106.5 (SEQ ID NO: 15592); ENST00000495852.6 (SEQ ID NO: 12300) |
| transcript | ENST00000370360.8 (SEQ ID NO: 12292); ENST00000463560.1 (SEQ ID NO: 12294); |
| | ENST00000471465.1 (SEQ ID NO: 12296); ENST00000487911.1 (SEQ ID NO: 12298); |
| | ENST00000495106.5 (SEQ ID NO: 12299); ENST00000495852.6 (SEQ ID NO: 12301) |

Target: GON4L

| | |
|---|---|
| pre-mRNA | ENST00000271883.9 (SEQ ID NO: 15593); ENST00000361040.9 (SEQ ID NO: 15594); |
| transcript | ENST00000368331.5 (SEQ ID NO: 15595); ENST00000437809.5 (SEQ ID NO: 15596); |
| | ENST00000460075.1 (SEQ ID NO: 12306); ENST00000466224.1 (SEQ ID NO: 12308); |
| | ENST00000467009.1 (SEQ ID NO: 15597); ENST00000468867.1 (SEQ ID NO: 15598); |
| | ENST00000471341.5 (SEQ ID NO: 15599); ENST00000473267.1 (SEQ ID NO: 12313); |
| | ENST00000482386.5 (SEQ ID NO: 15600); ENST00000483032.1 (SEQ ID NO: 12316); |
| | ENST00000488251.1 (SEQ ID NO: 12318); ENST00000490801.1 (SEQ ID NO: 12320); |
| | ENST00000496021.1 (SEQ ID NO: 12322); ENST00000497369.5 (SEQ ID NO: 12324); |
| | ENST00000615926.4 (SEQ ID NO: 15601); ENST00000620426.4 (SEQ ID NO: 15602); |
| | ENST00000622608.1 (SEQ ID NO: 15603) |
| transcript | ENST00000271883.9 (SEQ ID NO: 12302); ENST00000361040.9 (SEQ ID NO: 12303); |
| | ENST00000368331.5 (SEQ ID NO: 12304); ENST00000437809.5 (SEQ ID NO: 12305); |
| | ENST00000460075.1 (SEQ ID NO: 12307); ENST00000466224.1 (SEQ ID NO: 12309); |
| | ENST00000467009.1 (SEQ ID NO: 12310); ENST00000468867.1 (SEQ ID NO: 12311); |
| | ENST00000471341.5 (SEQ ID NO: 12312); ENST00000473267.1 (SEQ ID NO: 12314); |
| | ENST00000482386.5 (SEQ ID NO: 12315); ENST00000483032.1 (SEQ ID NO: 12317); |
| | ENST00000488251.1 (SEQ ID NO: 12319); ENST00000490801.1 (SEQ ID NO: 12321); |
| | ENST00000496021.1 (SEQ ID NO: 12323); ENST00000497369.5 (SEQ ID NO: 12325); |
| | ENST00000615926.4 (SEQ ID NO: 12326); ENST00000620426.4 (SEQ ID NO: 12327); |
| | ENST00000622608.1 (SEQ ID NO: 12328) |

Target: GRB14

| | |
|---|---|
| pre-mRNA | ENST00000263915.8 (SEQ ID NO: 15604); ENST00000424693.1 (SEQ ID NO: 15605); |
| transcript | ENST00000446413.6 (SEQ ID NO: 15606); ENST00000469573.1 (SEQ ID NO: 15607); |
| | ENST00000488342.5 (SEQ ID NO: 15608); ENST00000497306.1 (SEQ ID NO: 12334) |
| transcript | ENST00000263915.8 (SEQ ID NO: 12329); ENST00000424693.1 (SEQ ID NO: 12330); |
| | ENST00000446413.6 (SEQ ID NO: 12331); ENST00000469573.1 (SEQ ID NO: 12332); |
| | ENST00000488342.5 (SEQ ID NO: 12333); ENST00000497306.1 (SEQ ID NO: 12335) |

Target: HDAC3

| | |
|---|---|
| pre-mRNA | ENST00000305264.8 (SEQ ID NO: 12336); ENST00000459727.5 (SEQ ID NO: 12338); |
| transcript | ENST00000467533.5 (SEQ ID NO: 12340); ENST00000469207.5 (SEQ ID NO: 12342); |
| | ENST00000469550.6 (SEQ ID NO: 12344); ENST00000471968.1 (SEQ ID NO: 12346); |
| | ENST00000475549.1 (SEQ ID NO: 12348); ENST00000476739.5 (SEQ ID NO: 12350); |
| | ENST00000486618.1 (SEQ ID NO: 12352); ENST00000490808.1 (SEQ ID NO: 12354); |
| | ENST00000491581.5 (SEQ ID NO: 12356); ENST00000492407.1 (SEQ ID NO: 12358); |
| | ENST00000492506.1 (SEQ ID NO: 12360); ENST00000495485.1 (SEQ ID NO: 12362); |
| | ENST00000519474.5 (SEQ ID NO: 12364); ENST00000523088.5 (SEQ ID NO: 12366) |
| transcript | ENST00000305264.8 (SEQ ID NO: 12337); ENST00000459727.5 (SEQ ID NO: 12339); |
| | ENST00000467533.5 (SEQ ID NO: 12341); ENST00000469207.5 (SEQ ID NO: 12343); |
| | ENST00000469550.6 (SEQ ID NO: 12345); ENST00000471968.1 (SEQ ID NO: 12347); |
| | ENST00000475549.1 (SEQ ID NO: 12349); ENST00000476739.5 (SEQ ID NO: 12351); |
| | ENST00000486618.1 (SEQ ID NO: 12353); ENST00000490808.1 (SEQ ID NO: 12355); |
| | ENST00000491581.5 (SEQ ID NO: 12357); ENST00000492407.1 (SEQ ID NO: 12359); |
| | ENST00000492506.1 (SEQ ID NO: 12361); ENST00000495485.1 (SEQ ID NO: 12363); |
| | ENST00000519474.5 (SEQ ID NO: 12365); ENST00000523088.5 (SEQ ID NO: 12367) |

Target: HELQ

| | |
|---|---|
| pre-mRNA | ENST00000295488.8 (SEQ ID NO: 15609); ENST00000440639.2 (SEQ ID NO: 12369); |
| transcript | ENST00000508591.5 (SEQ ID NO: 15610); ENST00000510985.1 (SEQ ID NO: 15611); |
| | ENST00000512539.1 (SEQ ID NO: 12373); ENST00000515482.1; (SEQ ID NO: 12375) |
| transcript | ENST00000295488.8 (SEQ ID NO: 12368); ENST00000440639.2 (SEQ ID NO: 12370); |
| | ENST00000508591.5 (SEQ ID NO: 12371); ENST00000510985.1 (SEQ ID NO: 12372); |
| | ENST00000512539.1 (SEQ ID NO: 12374); ENST00000515482.1 (SEQ ID NO: 12376) |

Target: HP1BP3

| | |
|---|---|
| pre-mRNA | ENST00000312239.10 (SEQ ID NO: 15612); ENST00000375000.5 (SEQ ID NO: 12378); |
| transcript | ENST00000375003.6 (SEQ ID NO: 15613); ENST00000414993.1 (SEQ ID NO: 12381); |
| | ENST00000417710.5 (SEQ ID NO: 12383); ENST00000419490.5 (SEQ ID NO: 12385); |
| | ENST00000419948.5 (SEQ ID NO: 12387); ENST00000424732.5 (SEQ ID NO: 15614); |
| | ENST00000437575.5 (SEQ ID NO: 12390); ENST00000438032.6 (SEQ ID NO: 15615); |
| | ENST00000443615.1 (SEQ ID NO: 12393); ENST00000487117.1 (SEQ ID NO: 12395); |
| | ENST00000488722.5 (SEQ ID NO: 12397); ENST00000491748.1 (SEQ ID NO: 12399) |
| transcript | ENST00000312239.10 (SEQ ID NO: 12377); ENST00000375000.5 (SEQ ID NO: 12379); |
| | ENST00000375003.6 (SEQ ID NO: 12380); ENST00000414993.1 (SEQ ID NO: 12382); |
| | ENST00000417710.5 (SEQ ID NO: 12384); ENST00000419490.5 (SEQ ID NO: 12386); |
| | ENST00000419948.5 (SEQ ID NO: 12388); ENST00000424732.5 (SEQ ID NO: 12389); |
| | ENST00000437575.5 (SEQ ID NO: 12391); ENST00000438032.6 (SEQ ID NO: 12392); |
| | ENST00000443615.1 (SEQ ID NO: 12394); ENST00000487117.1 (SEQ ID NO: 12396); |
| | ENST00000488722.5 (SEQ ID NO: 12398); ENST00000491748.1 (SEQ ID NO: 12400) |

Target: HPS1

| | |
|---|---|
| pre-mRNA | ENST00000325103.10 (SEQ ID NO: 15616); ENST00000338546.9 (SEQ ID NO: 12402); |
| transcript | ENST00000359632.7 (SEQ ID NO: 12404); ENST00000361490.9 (SEQ ID NO: 15617); |
| | ENST00000414009.1 (SEQ ID NO: 12407); ENST00000462743.1 (SEQ ID NO: 12409); |
| | ENST00000465957.1 (SEQ ID NO: 12411); ENST00000467246.5 (SEQ ID NO: 12413); |
| | ENST00000470095.5 (SEQ ID NO: 12415); ENST00000474873.5 (SEQ ID NO: 12417); |
| | ENST00000478087.5 (SEQ ID NO: 12419); ENST00000480020.5 (SEQ ID NO: 12421); |
| | ENST00000497527.1 (SEQ ID NO: 12423); ENST00000498219.5 (SEQ ID NO: 12425); |
| | ENST00000613394.4 (SEQ ID NO: 15618) |
| transcript | ENST00000325103.10 (SEQ ID NO: 12401); ENST00000338546.9 (SEQ ID NO: 12403); |
| | ENST00000359632.7 (SEQ ID NO: 12405); ENST00000361490.9 (SEQ ID NO: 12406); |
| | ENST00000414009.1 (SEQ ID NO: 12408); ENST00000462743.1 (SEQ ID NO: 12410); |
| | ENST00000465957.1 (SEQ ID NO: 12412); ENST00000467246.5 (SEQ ID NO: 12414); |
| | ENST00000470095.5 (SEQ ID NO: 12416); ENST00000474873.5 (SEQ ID NO: 12418); |
| | ENST00000478087.5 (SEQ ID NO: 12420); ENST00000480020.5 (SEQ ID NO: 12422); |
| | ENST00000497527.1 (SEQ ID NO: 12424); ENST00000498219.5 (SEQ ID NO: 12426); |
| | ENST00000613394.4 (SEQ ID NO: 12427) |

Target: HPS4

| | |
|---|---|
| pre-mRNA | ENST00000336873.9 (SEQ ID NO: 15619); ENST00000398145.6 (SEQ ID NO: 15620); |
| transcript | ENST00000402105.7 (SEQ ID NO: 12430); ENST00000422379.2 (SEQ ID NO: 12432); |
| | ENST00000429411.5 (SEQ ID NO: 12434); ENST00000439453.5 (SEQ ID NO: 15621); |
| | ENST00000459918.1 (SEQ ID NO: 12437); ENST00000464362.5 (SEQ ID NO: 12439); |
| | ENST00000466781.5 (SEQ ID NO: 15622); ENST00000473782.1 (SEQ ID NO: 12442); |
| | ENST00000479064.5 (SEQ ID NO: 12444); ENST00000481910.1 (SEQ ID NO: 12446); |
| | ENST00000483631.1 (SEQ ID NO: 12448); ENST00000485842.5 (SEQ ID NO: 12450); |
| | ENST00000491142.1 (SEQ ID NO: 12452); ENST00000493455.6 (SEQ ID NO: 12454); |
| | ENST00000496385.5 (SEQ ID NO: 12456); ENST00000519774.5 (SEQ ID NO: 12458) |
| transcript | ENST00000336873.9 (SEQ ID NO: 12428); ENST00000398145.6 (SEQ ID NO: 12429); |
| | ENST00000402105.7 (SEQ ID NO: 12431); ENST00000422379.2 (SEQ ID NO: 12433); |
| | ENST00000429411.5 (SEQ ID NO: 12435); ENST00000439453.5 (SEQ ID NO: 12436); |
| | ENST00000459918.1 (SEQ ID NO: 12438); ENST00000464362.5 (SEQ ID NO: 12440); |
| | ENST00000466781.5 (SEQ ID NO: 12441); ENST00000473782.1 (SEQ ID NO: 12443); |
| | ENST00000479064.5 (SEQ ID NO: 12445); ENST00000481910.1 (SEQ ID NO: 12447); |
| | ENST00000483631.1 (SEQ ID NO: 12449); ENST00000485842.5 (SEQ ID NO: 12451); |
| | ENST00000491142.1 (SEQ ID NO: 12453); ENST00000493455.6 (SEQ ID NO: 12455); |
| | ENST00000496385.5 (SEQ ID NO: 12457); ENST00000519774.5 (SEQ ID NO: 12459) |

Target: IDUA

| | |
|---|---|
| pre-mRNA | ENST00000247933.9 (SEQ ID NO: 12460); ENST00000502829.1 (SEQ ID NO: 12462); |
| transcript | ENST00000502910.5 (SEQ ID NO: 12464); ENST00000504568.5 (SEQ ID NO: 12466); |
| | ENST00000506561.5 (SEQ ID NO: 12468); ENST00000508168.5 (SEQ ID NO: 12470); |
| | ENST00000509744.1 (SEQ ID NO: 12472); ENST00000509948.5 (SEQ ID NO: 12474); |
| | ENST00000514192.5 (SEQ ID NO: 12476); ENST00000514224.2 (SEQ ID NO: 12478); |
| | ENST00000514417.1 (SEQ ID NO: 12480); ENST00000514698.5 (SEQ ID NO: 12482); |
| | ENST00000652070.1 (SEQ ID NO: 12484) |
| transcript | ENST00000247933.9 (SEQ ID NO: 12461); ENST00000502829.1 (SEQ ID NO: 12463); |
| | ENST00000502910.5 (SEQ ID NO: 12465); ENST00000504568.5 (SEQ ID NO: 12467); |
| | ENST00000506561.5 (SEQ ID NO: 12469); ENST00000508168.5 (SEQ ID NO: 12471); |
| | ENST00000509744.1 (SEQ ID NO: 12473); ENST00000509948.5 (SEQ ID NO: 12475); |
| | ENST00000514192.5 (SEQ ID NO: 12477); ENST00000514224.2 (SEQ ID NO: 12479); |
| | ENST00000514417.1 (SEQ ID NO: 12481); ENST00000514698.5 (SEQ ID NO: 12483); |
| | ENST00000652070.1 (SEQ ID NO: 12485) |

Target: IFT122

| | |
|---|---|
| pre-mRNA | ENST00000296266.7 (SEQ ID NO: 15623); ENST00000347300.6 (SEQ ID NO: 15624); |
| transcript | ENST00000348417.6 (SEQ ID NO: 15625); ENST00000349441.6 (SEQ ID NO: 15626); |
| | ENST00000431818.6 (SEQ ID NO: 15627); ENST00000440957.6 (SEQ ID NO: 15628); |
| | ENST00000448668.2 (SEQ ID NO: 12492); ENST00000502304.5 (SEQ ID NO: 12494); |
| | ENST00000502456.5 (SEQ ID NO: 12496); ENST00000504021.5 (SEQ ID NO: 15629); |
| | ENST00000504444.1 (SEQ ID NO: 12499); ENST00000504653.5 (SEQ ID NO: 12501); |
| | ENST00000506507.5 (SEQ ID NO: 15630); ENST00000507221.1 (SEQ ID NO: 12504); |
| | ENST00000507564.5 (SEQ ID NO: 15631); ENST00000508654.1 (SEQ ID NO: 12507); |
| | ENST00000508826.5 (SEQ ID NO: 12509); ENST00000509195.5 (SEQ ID NO: 12511); |
| | ENST00000509522.5 (SEQ ID NO: 12513); ENST00000509815.1 (SEQ ID NO: 12515); |
| | ENST00000510524.5 (SEQ ID NO: 12517); ENST00000511425.5 (SEQ ID NO: 15632); |
| | ENST00000511498.1 (SEQ ID NO: 12520); ENST00000512157.5 (SEQ ID NO: 12522); |
| | ENST00000512220.5 (SEQ ID NO: 15633); ENST00000512814.5 (SEQ ID NO: 12525); |
| | ENST00000513190.1 (SEQ ID NO: 12527); ENST00000513891.1 (SEQ ID NO: 12529); |
| | ENST00000513932.1 (SEQ ID NO: 12531); ENST00000514081.1 (SEQ ID NO: 12533); |
| | ENST00000514275.5 (SEQ ID NO: 12535); ENST00000515783.5 (SEQ ID NO: 15634) |
| transcript | ENST00000296266.7 (SEQ ID NO: 12486); ENST00000347300.6 (SEQ ID NO: 12487); |
| | ENST00000348417.6 (SEQ ID NO: 12488); ENST00000349441.6 (SEQ ID NO: 12489); |
| | ENST00000431818.6 (SEQ ID NO: 12490); ENST00000440957.6 (SEQ ID NO: 12491); |
| | ENST00000448668.2 (SEQ ID NO: 12493); ENST00000502304.5 (SEQ ID NO: 12495); |
| | ENST00000502456.5 (SEQ ID NO: 12497); ENST00000504021.5 (SEQ ID NO: 12498); |
| | ENST00000504444.1 (SEQ ID NO: 12500); ENST00000504653.5 (SEQ ID NO: 12502); |
| | ENST00000506507.5 (SEQ ID NO: 12503); ENST00000507221.1 (SEQ ID NO: 12505); |
| | ENST00000507564.5 (SEQ ID NO: 12506); ENST00000508654.1 (SEQ ID NO: 12508); |
| | ENST00000508826.5 (SEQ ID NO: 12510); ENST00000509195.5 (SEQ ID NO: 12512); |
| | ENST00000509522.5 (SEQ ID NO: 12514); ENST00000509815.1 (SEQ ID NO: 12516); |
| | ENST00000510524.5 (SEQ ID NO: 12518); ENST00000511425.5 (SEQ ID NO: 12519); |
| | ENST00000511498.1 (SEQ ID NO: 12521); ENST00000512157.5 (SEQ ID NO: 12523); |
| | ENST00000512220.5 (SEQ ID NO: 12524); ENST00000512814.5 (SEQ ID NO: 12526); |
| | ENST00000513190.1 (SEQ ID NO: 12528); ENST00000513891.1 (SEQ ID NO: 12530); |
| | ENST00000513932.1 (SEQ ID NO: 12532); ENST00000514081.1 (SEQ ID NO: 12534); |
| | ENST00000514275.5 (SEQ ID NO: 12536); ENST00000515783.5 (SEQ ID NO: 12537) |

Target: IL17RC

| | |
|---|---|
| pre-mRNA | ENST00000295981.7 (SEQ ID NO: 12538); ENST00000383812.9 (SEQ ID NO: 12540); |
| transcript | ENST00000403601.8 (SEQ ID NO: 12542); ENST00000412901.5 (SEQ ID NO: 12544); |
| | ENST00000413608.2 (SEQ ID NO: 12546); ENST00000416074.6 (SEQ ID NO: 12548); |
| | ENST00000424206.5 (SEQ ID NO: 12550); ENST00000434756.5 (SEQ ID NO: 12552); |
| | ENST00000436503.5 (SEQ ID NO: 12554); ENST00000438091.5 (SEQ ID NO: 12556); |
| | ENST00000440502.5 (SEQ ID NO: 12558); ENST00000451165.6 (SEQ ID NO: 12560); |
| | ENST00000451231.5 (SEQ ID NO: 12562); ENST00000451271.5 (SEQ ID NO: 12564); |
| | ENST00000455057.5 (SEQ ID NO: 12566); ENST00000461995.5 (SEQ ID NO: 12568); |
| | ENST00000464406.5 (SEQ ID NO: 12570); ENST00000465794.1 (SEQ ID NO: 12572); |
| | ENST00000466046.5 (SEQ ID NO: 12574); ENST00000466712.1 (SEQ ID NO: 12576); |
| | ENST00000469686.5 (SEQ ID NO: 12578); ENST00000476810.5 (SEQ ID NO: 12580); |
| | ENST00000478206.1 (SEQ ID NO: 12582); ENST00000481032.1 (SEQ ID NO: 12584); |
| | ENST00000483582.5 (SEQ ID NO: 12586); ENST00000490512.5 (SEQ ID NO: 12588); |
| | ENST00000494365.5 (SEQ ID NO: 12590); ENST00000497102.5 (SEQ ID NO: 12592); |
| | ENST00000497387.5 (SEQ ID NO: 12594); ENST00000498214.6 (SEQ ID NO: 12596) |
| transcript | ENST00000295981.7 (SEQ ID NO: 12539); ENST00000383812.9 (SEQ ID NO: 12541); |
| | ENST00000403601.8 (SEQ ID NO: 12543); ENST00000412901.5 (SEQ ID NO: 12545); |
| | ENST00000413608.2 (SEQ ID NO: 12547); ENST00000416074.6 (SEQ ID NO: 12549); |
| | ENST00000424206.5 (SEQ ID NO: 12551); ENST00000434756.5 (SEQ ID NO: 12553); |
| | ENST00000436503.5 (SEQ ID NO: 12555); ENST00000438091.5 (SEQ ID NO: 12557); |
| | ENST00000440502.5 (SEQ ID NO: 12559); ENST00000451165.6 (SEQ ID NO: 12561); |
| | ENST00000451231.5 (SEQ ID NO: 12563); ENST00000451271.5 (SEQ ID NO: 12565); |
| | ENST00000455057.5 (SEQ ID NO: 12567); ENST00000461995.5 (SEQ ID NO: 12569); |
| | ENST00000464406.5 (SEQ ID NO: 12571); ENST00000465794.1 (SEQ ID NO: 12573); |
| | ENST00000466046.5 (SEQ ID NO: 12575); ENST00000466712.1 (SEQ ID NO: 12577); |
| | ENST00000469686.5 (SEQ ID NO: 12579); ENST00000476810.5 (SEQ ID NO: 12581); |
| | ENST00000478206.1 (SEQ ID NO: 12583); ENST00000481032.1 (SEQ ID NO: 12585); |
| | ENST00000483582.5 (SEQ ID NO: 12587); ENST00000490512.5 (SEQ ID NO: 12589); |
| | ENST00000494365.5 (SEQ ID NO: 12591); ENST00000497102.5 (SEQ ID NO: 12593); |
| | ENST00000497387.5 (SEQ ID NO: 12595); ENST00000498214.6 (SEQ ID NO: 12597) |

Target: IMPDH1

| | |
|---|---|
| pre-mRNA | ENST00000338791.11 (SEQ ID NO: 12598); ENST00000348127.10 (SEQ ID NO: 12600); |
| transcript | ENST00000354269.9 (SEQ ID NO: 12602); ENST00000419067.6 (SEQ ID NO: 12604); |
| | ENST00000460045.1 (SEQ ID NO: 12606); ENST00000468842.1 (SEQ ID NO: 12608); |
| | ENST00000469328.5 (SEQ ID NO: 12610); ENST00000470772.5 (SEQ ID NO: 12612); |
| | ENST00000473463.1 (SEQ ID NO: 12614); ENST00000480861.5 (SEQ ID NO: 12616); |
| | ENST00000484496.5 (SEQ ID NO: 12618); ENST00000489263.1 (SEQ ID NO: 12620); |
| | ENST00000491376.5 (SEQ ID NO: 12622); ENST00000496200.5 (SEQ ID NO: 12624); |
| | ENST00000496487.5 (SEQ ID NO: 12626); ENST00000497868.5 (SEQ ID NO: 12628); |
| | ENST00000626419.2 (SEQ ID NO: 12630); ENST00000648462.1 (SEQ ID NO: 12632) |
| transcript | ENST00000338791.11 (SEQ ID NO: 12599); ENST00000348127.10 (SEQ ID NO: 12601); |
| | ENST00000354269.9 (SEQ ID NO: 12603); ENST00000419067.6 (SEQ ID NO: 12605); |
| | ENST00000460045.1 (SEQ ID NO: 12607); ENST00000468842.1 (SEQ ID NO: 12609); |
| | ENST00000469328.5 (SEQ ID NO: 12611); ENST00000470772.5 (SEQ ID NO: 12613); |
| | ENST00000473463.1 (SEQ ID NO: 12615); ENST00000480861.5 (SEQ ID NO: 12617); |
| | ENST00000484496.5 (SEQ ID NO: 12619); ENST00000489263.1 (SEQ ID NO: 12621); |
| | ENST00000491376.5 (SEQ ID NO: 12623); ENST00000496200.5 (SEQ ID NO: 12625); |
| | ENST00000496487.5 (SEQ ID NO: 12627); ENST00000497868.5 (SEQ ID NO: 12629); |
| | ENST00000626419.2 (SEQ ID NO: 12631); ENST00000648462.1 (SEQ ID NO: 12633) |

Target: INVS

| | |
|---|---|
| pre-mRNA | ENST00000262456.6 (SEQ ID NO: 15635); ENST00000262457.7 (SEQ ID NO: 15636); |
| transcript | ENST00000374921.3 (SEQ ID NO: 15637); ENST00000460636.2 (SEQ ID NO: 15638); |
| | ENST00000466647.5 (SEQ ID NO: 15639); ENST00000480309.1 (SEQ ID NO: 12639); |
| | ENST00000496467.5 (SEQ ID NO: 15640) |
| transcript | ENST00000262456.6 (SEQ ID NO: 12634); ENST00000262457.7 (SEQ ID NO: 12635); |
| | ENST00000374921.3 (SEQ ID NO: 12636); ENST00000460636.2 (SEQ ID NO: 12637); |
| | ENST00000466647.5 (SEQ ID NO: 12638); ENST00000480309.1 (SEQ ID NO: 12640); |
| | ENST00000496467.5 (SEQ ID NO: 12641) |

Target: IRF3

| | |
|---|---|
| pre-mRNA | ENST00000309877.11 (SEQ ID NO: 12642); ENST00000377135.8 (SEQ ID NO: 12644); |
| transcript | ENST00000377139.8 (SEQ ID NO: 12646); ENST00000442265.2 (SEQ ID NO: 12648); |
| | ENST00000593337.5 (SEQ ID NO: 12650); ENST00000593818.5 (SEQ ID NO: 12652); |
| | ENST00000593922.5 (SEQ ID NO: 12654); ENST00000594387.1 (SEQ ID NO: 12656); |
| | ENST00000595034.5 (SEQ ID NO: 12658); ENST00000595240.5 (SEQ ID NO: 12660); |
| | ENST00000596644.5 (SEQ ID NO: 12662); ENST00000596756.5 (SEQ ID NO: 12664); |
| | ENST00000596765.5 (SEQ ID NO: 12666); ENST00000596788.1 (SEQ ID NO: 12668); |
| | ENST00000596822.5 (SEQ ID NO: 12670); ENST00000597180.1 (SEQ ID NO: 12672); |
| | ENST00000597198.5 (SEQ ID NO: 12674); ENST00000597369.1 (SEQ ID NO: 12676); |
| | ENST00000597636.5 (SEQ ID NO: 12678); ENST00000598108.5 (SEQ ID NO: 12680); |
| | ENST00000598808.5 (SEQ ID NO: 12682); ENST00000599144.5 (SEQ ID NO: 12684); |
| | ENST00000599223.5 (SEQ ID NO: 12686); ENST00000599680.1 (SEQ ID NO: 12688); |
| | ENST00000600022.5 (SEQ ID NO: 12690); ENST00000600453.1 (SEQ ID NO: 12692); |
| | ENST00000600911.5 (SEQ ID NO: 12694); ENST00000601291.5 (SEQ ID NO: 12696); |
| | ENST00000601373.5 (SEQ ID NO: 12698); ENST00000601809.5 (SEQ ID NO: 12700); |
| | ENST00000602190.1 (SEQ ID NO: 12702) |
| transcript | ENST00000309877.11 (SEQ ID NO: 12643); ENST00000377135.8 (SEQ ID NO: 12645); |
| | ENST00000377139.8 (SEQ ID NO: 12647); ENST00000442265.2 (SEQ ID NO: 12649); |
| | ENST00000593337.5 (SEQ ID NO: 12651); ENST00000593818.5 (SEQ ID NO: 12653); |
| | ENST00000593922.5 (SEQ ID NO: 12655); ENST00000594387.1 (SEQ ID NO: 12657); |
| | ENST00000595034.5 (SEQ ID NO: 12659); ENST00000595240.5 (SEQ ID NO: 12661); |
| | ENST00000596644.5 (SEQ ID NO: 12663); ENST00000596756.5 (SEQ ID NO: 12665); |
| | ENST00000596765.5 (SEQ ID NO: 12667); ENST00000596788.1 (SEQ ID NO: 12669); |
| | ENST00000596822.5 (SEQ ID NO: 12671); ENST00000597180.1 (SEQ ID NO: 12673); |
| | ENST00000597198.5 (SEQ ID NO: 12675); ENST00000597369.1 (SEQ ID NO: 12677); |
| | ENST00000597636.5 (SEQ ID NO: 12679); ENST00000598108.5 (SEQ ID NO: 12681); |
| | ENST00000598808.5 (SEQ ID NO: 12683); ENST00000599144.5 (SEQ ID NO: 12685); |
| | ENST00000599223.5 (SEQ ID NO: 12687); ENST00000599680.1 (SEQ ID NO: 12689); |
| | ENST00000600022.5 (SEQ ID NO: 12691); ENST00000600453.1 (SEQ ID NO: 12693); |
| | ENST00000600911.5 (SEQ ID NO: 12695); ENST00000601291.5 (SEQ ID NO: 12697); |
| | ENST00000601373.5 (SEQ ID NO: 12699); ENST00000601809.5 (SEQ ID NO: 12701); |
| | ENST00000602190.1 (SEQ ID NO: 12703) |

Target: KCTD3

| | |
|---|---|
| pre-mRNA | ENST00000259154.9 (SEQ ID NO: 15641); ENST00000448333.1 (SEQ ID NO: 12705); |
| transcript | ENST00000452413.1 (SEQ ID NO: 12707); ENST00000465650.1 (SEQ ID NO: 12709); |
| | ENST00000495537.1 (SEQ ID NO: 12711) |
| transcript | ENST00000259154.9 (SEQ ID NO: 12704); ENST00000448333.1 (SEQ ID NO: 12706); |
| | ENST00000452413.1 (SEQ ID NO: 12708); ENST00000465650.1 (SEQ ID NO: 12710); |
| | ENST00000495537.1 (SEQ ID NO: 12712) |

Target: KLKB1

| | |
|---|---|
| pre-mRNA | ENST00000264690.11 (SEQ ID NO: 15642); ENST00000428196.5 (SEQ ID NO: 12714); |
| transcript | ENST00000446598.6 (SEQ ID NO: 12716); ENST00000467271.1 (SEQ ID NO: 12718); |
| | ENST00000511406.5 (SEQ ID NO: 15643); ENST00000511608.5 (SEQ ID NO: 15644); |
| | ENST00000513864.2 (SEQ ID NO: 15645) |
| transcript | ENST00000264690.11 (SEQ ID NO: 12713); ENST00000428196.5 (SEQ ID NO: 12715); |
| | ENST00000446598.6 (SEQ ID NO: 12717); ENST00000467271.1 (SEQ ID NO: 12719); |
| | ENST00000511406.5 (SEQ ID NO: 12720); ENST00000511608.5 (SEQ ID NO: 12721); |
| | ENST00000513864.2 (SEQ ID NO: 12722) |

Target: KYAT1

| | |
|---|---|
| pre-mRNA | ENST00000302586.8 (SEQ ID NO: 15646); ENST00000320665.10 (SEQ ID NO: 15647); |
| transcript | ENST00000416084.5 (SEQ ID NO: 12725); ENST00000427720.1 (SEQ ID NO: 12727); |
| | ENST00000436267.7 (SEQ ID NO: 15648); ENST00000451800.5 (SEQ ID NO: 15649); |
| | ENST00000462722.5 (SEQ ID NO: 15650); ENST00000466418.1 (SEQ ID NO: 12732); |
| | ENST00000474824.1 (SEQ ID NO: 15651); ENST00000483599.5 (SEQ ID NO: 12735) |
| transcript | ENST00000302586.8 (SEQ ID NO: 12723); ENST00000320665.10 (SEQ ID NO: 12724); |
| | ENST00000416084.5 (SEQ ID NO: 12726); ENST00000427720.1 (SEQ ID NO: 12728); |
| | ENST00000436267.7 (SEQ ID NO: 12729); ENST00000451800.5 (SEQ ID NO: 12730); |
| | ENST00000462722.5 (SEQ ID NO: 12731); ENST00000466418.1 (SEQ ID NO: 12733); |
| | ENST00000474824.1 (SEQ ID NO: 12734); ENST00000483599.5 (SEQ ID NO: 12736) |

Target: LAMC3

| | |
|---|---|
| pre-mRNA | ENST00000355452.5 (SEQ ID NO: 12737); ENST00000361069.9 (SEQ ID NO: 15652); |
| transcript | ENST00000462567.1 (SEQ ID NO: 12740); ENST00000480883.1 (SEQ ID NO: 15653) |
| transcript | ENST00000355452.5 (SEQ ID NO: 12738); ENST00000361069.9 (SEQ ID NO: 12739); |
| | ENST00000462567.1 (SEQ ID NO: 12741); ENST00000480883.1 (SEQ ID NO: 12742) |

Target: LDAH

| | |
|---|---|
| pre-mRNA | ENST00000237822.8 (SEQ ID NO: 15654); ENST00000381090.7 (SEQ ID NO: 15655); |
| transcript | ENST00000402479.6 (SEQ ID NO: 15656); ENST00000403006.6 (SEQ ID NO: 15657); |
| | ENST00000412261.5 (SEQ ID NO: 15658); ENST00000419825.2 (SEQ ID NO: 15659); |
| | ENST00000432947.1 (SEQ ID NO: 15660); ENST00000435420.6 (SEQ ID NO: 15661); |
| | ENST00000440866.6 (SEQ ID NO: 15662); ENST00000470099.1 (SEQ ID NO: 15663); |
| | ENST00000541941.5 (SEQ ID NO: 15664); ENST00000619656.4 (SEQ ID NO: 15665); |
| | ENST00000626491.2 (SEQ ID NO: 15666) |
| transcript | ENST00000237822.8 (SEQ ID NO: 12743); ENST00000381090.7 (SEQ ID NO: 12744); |
| | ENST00000402479.6 (SEQ ID NO: 12745); ENST00000403006.6 (SEQ ID NO: 12746); |
| | ENST00000412261.5 (SEQ ID NO: 12747); ENST00000419825.2 (SEQ ID NO: 12748); |
| | ENST00000432947.1 (SEQ ID NO: 12749); ENST00000435420.6 (SEQ ID NO: 12750); |
| | ENST00000440866.6 (SEQ ID NO: 12751); ENST00000470099.1 (SEQ ID NO: 12752); |
| | ENST00000541941.5 (SEQ ID NO: 12753); ENST00000619656.4 (SEQ ID NO: 12754); |
| | ENST00000626491.2 (SEQ ID NO: 12755) |

Target: LIMS2

| | |
|---|---|
| pre-mRNA | ENST00000324938.9 (SEQ ID NO: 12756); ENST00000355119.8 (SEQ ID NO: 15667); |
| transcript | ENST00000409254.1 (SEQ ID NO: 12759); ENST00000409286.5 (SEQ ID NO: 12761); |
| | ENST00000409455.5 (SEQ ID NO: 15668); ENST00000409754.5 (SEQ ID NO: 12764); |
| | ENST00000409808.6 (SEQ ID NO: 15669); ENST00000410011.5 (SEQ ID NO: 15670); |
| | ENST00000410038.5 (SEQ ID NO: 12768); ENST00000413578.5 (SEQ ID NO: 12770); |
| | ENST00000426981.5 (SEQ ID NO: 12772); ENST00000466410.5 (SEQ ID NO: 12774); |
| | ENST00000469300.6 (SEQ ID NO: 12776); ENST00000476932.5 (SEQ ID NO: 12778); |
| | ENST00000484252.5 (SEQ ID NO: 12780); ENST00000494613.5 (SEQ ID NO: 12782); |
| | ENST00000545738.6 (SEQ ID NO: 15671); ENST00000582671.1 (SEQ ID NO: 12785) |
| transcript | ENST00000324938.9 (SEQ ID NO: 12757); ENST00000355119.8 (SEQ ID NO: 12758); |
| | ENST00000409254.1 (SEQ ID NO: 12760); ENST00000409286.5 (SEQ ID NO: 12762); |
| | ENST00000409455.5 (SEQ ID NO: 12763); ENST00000409754.5 (SEQ ID NO: 12765); |
| | ENST00000409808.6 (SEQ ID NO: 12766); ENST00000410011.5 (SEQ ID NO: 12767); |
| | ENST00000410038.5 (SEQ ID NO: 12769); ENST00000413578.5 (SEQ ID NO: 12771); |
| | ENST00000426981.5 (SEQ ID NO: 12773); ENST00000466410.5 (SEQ ID NO: 12775); |
| | ENST00000469300.6 (SEQ ID NO: 12777); ENST00000476932.5 (SEQ ID NO: 12779); |
| | ENST00000484252.5 (SEQ ID NO: 12781); ENST00000494613.5 (SEQ ID NO: 12783); |
| | ENST00000545738.6 (SEQ ID NO: 12784); ENST00000582671.1 (SEQ ID NO: 12786) |

Target: MALT1

| | |
|---|---|
| pre-mRNA | ENST00000345724.7 (SEQ ID NO: 15672); ENST00000587438.1 (SEQ ID NO: 12788); |
| transcript | ENST00000589873.5 (SEQ ID NO: 12790); ENST00000591792.1 (SEQ ID NO: 12792); |
| | ENST00000648670.1 (SEQ ID NO: 15673); ENST00000649125.1 (SEQ ID NO: 15674); |
| | ENST00000649202.1 (SEQ ID NO: 15675); ENST00000649217.2 (SEQ ID NO: 15676); |
| | ENST00000649756.1 (SEQ ID NO: 12798); ENST00000650045.1 (SEQ ID NO: 15677); |
| | ENST00000650355.1; (SEQ ID NO: 12801) |
| transcript | ENST00000345724.7 (SEQ ID NO: 12787); ENST00000587438.1 (SEQ ID NO: 12789); |
| | ENST00000589873.5 (SEQ ID NO: 12791); ENST00000591792.1 (SEQ ID NO: 12793); |
| | ENST00000648670.1 (SEQ ID NO: 12794); ENST00000649125.1 (SEQ ID NO: 12795); |
| | ENST00000649202.1 (SEQ ID NO: 12796); ENST00000649217.2 (SEQ ID NO: 12797); |
| | ENST00000649756.1 (SEQ ID NO: 12799); ENST00000650045.1 (SEQ ID NO: 12800); |
| | ENST00000650355.1 (SEQ ID NO: 12802) |

Target: MAP3K7

| | |
|---|---|
| pre-mRNA | ENST00000369320.1 (SEQ ID NO: 15678); ENST00000369325.7 (SEQ ID NO: 15679); |
| transcript | ENST00000369327.7 (SEQ ID NO: 15680); ENST00000369329.8 (SEQ ID NO: 15681); |
| | ENST00000369332.7 (SEQ ID NO: 15682); ENST00000479630.1 (SEQ ID NO: 12808) |
| transcript | ENST00000369320.1 (SEQ ID NO: 12803); ENST00000369325.7 (SEQ ID NO: 12804); |
| | ENST00000369327.7 (SEQ ID NO: 12805); ENST00000369329.8 (SEQ ID NO: 12806); |
| | ENST00000369332.7 (SEQ ID NO: 12807); ENST00000479630.1 (SEQ ID NO: 12809) |

Target: MAPK13

| | |
|---|---|
| pre-mRNA | ENST00000211287.9 (SEQ ID NO: 12810); ENST00000373759.1 (SEQ ID NO: 12812); |
| transcript | ENST00000373766.9 (SEQ ID NO: 12814); ENST00000476951.5 (SEQ ID NO: 12816); |
| | ENST00000490334.1 (SEQ ID NO: 12818) |
| transcript | ENST00000211287.9 (SEQ ID NO: 12811); ENST00000373759.1 (SEQ ID NO: 12813); |
| | ENST00000373766.9 (SEQ ID NO: 12815); ENST00000476951.5 (SEQ ID NO: 12817); |
| | ENST00000490334.1 (SEQ ID NO: 12819) |

Target: MCAT

| | |
|---|---|
| pre-mRNA | ENST00000290429.11 (SEQ ID NO: 12820); ENST00000327555.5 (SEQ ID NO: 12822); |
| transcript | ENST00000464244.1 (SEQ ID NO: 12824); ENST00000608052.1 (SEQ ID NO: 12826) |
| transcript | ENST00000290429.11 (SEQ ID NO: 12821); ENST00000327555.5 (SEQ ID NO: 12823); |
| | ENST00000464244.1 (SEQ ID NO: 12825); ENST00000608052.1 (SEQ ID NO: 12827) |

Target: MCEE

| | |
|---|---|
| pre-mRNA | ENST00000244217.6 (SEQ ID NO: 12828); ENST00000413592.5 (SEQ ID NO: 12830); |
| transcript | ENST00000462609.2 (SEQ ID NO: 12832); ENST00000486135.1 (SEQ ID NO: 12834); |
| | ENST00000494660.6 (SEQ ID NO: 12836) |
| transcript | ENST00000244217.6 (SEQ ID NO: 12829); ENST00000413592.5 (SEQ ID NO: 12831); |
| | ENST00000462609.2 (SEQ ID NO: 12833); ENST00000486135.1 (SEQ ID NO: 12835); |
| | ENST00000494660.6 (SEQ ID NO: 12837) |

Target: MPI

| | |
|---|---|
| pre-mRNA | ENST00000323744.10 (SEQ ID NO: 12838); ENST00000352410.9 (SEQ ID NO: 12840); |
| transcript | ENST00000535694.5 (SEQ ID NO: 12842); ENST00000561470.5 (SEQ ID NO: 12844); |
| | ENST00000562606.5 (SEQ ID NO: 12846); ENST00000562800.5 (SEQ ID NO: 12848); |
| | ENST00000563422.5 (SEQ ID NO: 12850); ENST00000563786.5 (SEQ ID NO: 12852); |
| | ENST00000564003.5 (SEQ ID NO: 12854); ENST00000564633.5 (SEQ ID NO: 12856); |
| | ENST00000564692.1 (SEQ ID NO: 12858); ENST00000565576.5 (SEQ ID NO: 12860); |
| | ENST00000566377.5 (SEQ ID NO: 12862); ENST00000566556.1 (SEQ ID NO: 12864); |
| | ENST00000567116.5 (SEQ ID NO: 12866); ENST00000567132.5 (SEQ ID NO: 12868); |
| | ENST00000567177.1 (SEQ ID NO: 12870); ENST00000567570.5 (SEQ ID NO: 12872); |
| | ENST00000568303.1 (SEQ ID NO: 12874); ENST00000568828.5 (SEQ ID NO: 12876); |
| | ENST00000568840.1 (SEQ ID NO: 12878); ENST00000568907.5 (SEQ ID NO: 12880); |
| | ENST00000569233.5 (SEQ ID NO: 12882); ENST00000569931.5 (SEQ ID NO: 12884) |
| transcript | ENST00000323744.10 (SEQ ID NO: 12839); ENST00000352410.9 (SEQ ID NO: 12841); |
| | ENST00000535694.5 (SEQ ID NO: 12843); ENST00000561470.5 (SEQ ID NO: 12845); |
| | ENST00000562606.5 (SEQ ID NO: 12847); ENST00000562800.5 (SEQ ID NO: 12849); |
| | ENST00000563422.5 (SEQ ID NO: 12851); ENST00000563786.5 (SEQ ID NO: 12853); |
| | ENST00000564003.5 (SEQ ID NO: 12855); ENST00000564633.5 (SEQ ID NO: 12857); |
| | ENST00000564692.1 (SEQ ID NO: 12859); ENST00000565576.5 (SEQ ID NO: 12861); |
| | ENST00000566377.5 (SEQ ID NO: 12863); ENST00000566556.1 (SEQ ID NO: 12865); |
| | ENST00000567116.5 (SEQ ID NO: 12867); ENST00000567132.5 (SEQ ID NO: 12869); |
| | ENST00000567177.1 (SEQ ID NO: 12871); ENST00000567570.5 (SEQ ID NO: 12873); |
| | ENST00000568303.1 (SEQ ID NO: 12875); ENST00000568828.5 (SEQ ID NO: 12877); |
| | ENST00000568840.1 (SEQ ID NO: 12879); ENST00000568907.5 (SEQ ID NO: 12881); |
| | ENST00000569233.5 (SEQ ID NO: 12883); ENST00000569931.5 (SEQ ID NO: 12885) |

Target: MSTO1

| | |
|---|---|
| pre-mRNA | ENST00000245564.7 (SEQ ID NO: 12886); ENST00000368341.8 (SEQ ID NO: 12888); |
| transcript | ENST00000460199.5 (SEQ ID NO: 12890); ENST00000462250.2 (SEQ ID NO: 12892); |
| | ENST00000465137.5 (SEQ ID NO: 12894); ENST00000466815.1 (SEQ ID NO: 12896); |
| | ENST00000471209.1 (SEQ ID NO: 12898); ENST00000473327.5 (SEQ ID NO: 12900); |
| | ENST00000475253.5 (SEQ ID NO: 12902); ENST00000478756.1 (SEQ ID NO: 12904); |
| | ENST00000482284.5 (SEQ ID NO: 12906); ENST00000483734.5 (SEQ ID NO: 12908); |
| | ENST00000483832.5 (SEQ ID NO: 12910); ENST00000488901.5 (SEQ ID NO: 12912); |
| | ENST00000490642.5 (SEQ ID NO: 12914); ENST00000490743.5 (SEQ ID NO: 12916); |
| | ENST00000491308.5 (SEQ ID NO: 12918); ENST00000494995.5 (SEQ ID NO: 12920); |
| | ENST00000649846.1 (SEQ ID NO: 12922) |
| transcript | ENST00000245564.7 (SEQ ID NO: 12887); ENST00000368341.8 (SEQ ID NO: 12889); |
| | ENST00000460199.5 (SEQ ID NO: 12891); ENST00000462250.2 (SEQ ID NO: 12893); |
| | ENST00000465137.5 (SEQ ID NO: 12895); ENST00000466815.1 (SEQ ID NO: 12897); |
| | ENST00000471209.1 (SEQ ID NO: 12899); ENST00000473327.5 (SEQ ID NO: 12901); |
| | ENST00000475253.5 (SEQ ID NO: 12903); ENST00000478756.1 (SEQ ID NO: 12905); |
| | ENST00000482284.5 (SEQ ID NO: 12907); ENST00000483734.5 (SEQ ID NO: 12909); |
| | ENST00000483832.5 (SEQ ID NO: 12911); ENST00000488901.5 (SEQ ID NO: 12913); |
| | ENST00000490642.5 (SEQ ID NO: 12915); ENST00000490743.5 (SEQ ID NO: 12917); |
| | ENST00000491308.5 (SEQ ID NO: 12919); ENST00000494995.5 (SEQ ID NO: 12921); |
| | ENST00000649846.1 (SEQ ID NO: 12923) |

Target: NBEAL2

| | |
|---|---|
| pre-mRNA | ENST00000416683.5 (SEQ ID NO: 12924); ENST00000423436.1 (SEQ ID NO: 12926); |
| transcript | ENST00000441027.5 (SEQ ID NO: 12928); ENST00000443829.5 (SEQ ID NO: 12930); |
| | ENST00000450053.8 (SEQ ID NO: 15683); ENST00000461036.1 (SEQ ID NO: 12933); |
| | ENST00000469349.1 (SEQ ID NO: 12935); ENST00000475689.1 (SEQ ID NO: 12937); |
| | ENST00000476095.5 (SEQ ID NO: 12939); ENST00000477412.1 (SEQ ID NO: 12941); |
| | ENST00000486870.1 (SEQ ID NO: 12943); ENST00000651350.1 (SEQ ID NO: 12945); |
| | ENST00000651450.1 (SEQ ID NO: 12947); ENST00000651453.1 (SEQ ID NO: 12949); |
| | ENST00000651747.1 (SEQ ID NO: 12951); ENST00000652242.1 (SEQ ID NO: 12953); |
| | ENST00000652744.1; (SEQ ID NO: 12955) |
| transcript | ENST00000416683.5 (SEQ ID NO: 12925); ENST00000423436.1 (SEQ ID NO: 12927); |
| | ENST00000441027.5 (SEQ ID NO: 12929); ENST00000443829.5 (SEQ ID NO: 12931); |
| | ENST00000450053.8 (SEQ ID NO: 12932); ENST00000461036.1 (SEQ ID NO: 12934); |
| | ENST00000469349.1 (SEQ ID NO: 12936); ENST00000475689.1 (SEQ ID NO: 12938); |
| | ENST00000476095.5 (SEQ ID NO: 12940); ENST00000477412.1 (SEQ ID NO: 12942); |
| | ENST00000486870.1 (SEQ ID NO: 12944); ENST00000651350.1 (SEQ ID NO: 12946); |
| | ENST00000651450.1 (SEQ ID NO: 12948); ENST00000651453.1 (SEQ ID NO: 12950); |
| | ENST00000651747.1 (SEQ ID NO: 12952); ENST00000652242.1 (SEQ ID NO: 12954); |
| | ENST00000652744.1 (SEQ ID NO: 12956) |

Target: NLE1

| | |
|---|---|
| pre-mRNA | ENST00000360831.9 (SEQ ID NO: 12957); ENST00000442241.9 (SEQ ID NO: 12959); |
| transcript | ENST00000586869.5 (SEQ ID NO: 12961); ENST00000588019.1 (SEQ ID NO: 12963); |
| | ENST00000588642.1 (SEQ ID NO: 12965); ENST00000589367.5 (SEQ ID NO: 12967); |
| | ENST00000593176.1; (SEQ ID NO: 12969) |
| transcript | ENST00000360831.9 (SEQ ID NO: 12958); ENST00000442241.9 (SEQ ID NO: 12960); |
| | ENST00000586869.5 (SEQ ID NO: 12962); ENST00000588019.1 (SEQ ID NO: 12964); |
| | ENST00000588642.1 (SEQ ID NO: 12966); ENST00000589367.5 (SEQ ID NO: 12968); |
| | ENST00000593176.1 (SEQ ID NO: 12970) |

Target: NLRC5

| | |
|---|---|
| pre-mRNA | ENST00000262510.10 (SEQ ID NO: 15684); ENST00000399221.3 (SEQ ID NO: 12972); |
| transcript | ENST00000436936.5 (SEQ ID NO: 15685); ENST00000534927.5 (SEQ ID NO: 12975); |
| | ENST00000534931.1 (SEQ ID NO: 12977); ENST00000535284.1 (SEQ ID NO: 12979); |
| | ENST00000535658.1 (SEQ ID NO: 12981); ENST00000536231.1 (SEQ ID NO: 12983); |
| | ENST00000537056.5 (SEQ ID NO: 15686); ENST00000538059.5 (SEQ ID NO: 12986); |
| | ENST00000538110.5 (SEQ ID NO: 15687); ENST00000538273.5 (SEQ ID NO: 15688); |
| | ENST00000538453.5 (SEQ ID NO: 15689); ENST00000538778.5 (SEQ ID NO: 12991); |
| | ENST00000538805.5 (SEQ ID NO: 15690); ENST00000538930.5 (SEQ ID NO: 12994); |
| | ENST00000539144.5 (SEQ ID NO: 15691); ENST00000539881.5 (SEQ ID NO: 15692); |
| | ENST00000540182.5 (SEQ ID NO: 15693); ENST00000541020.1 (SEQ ID NO: 12999); |
| | ENST00000543030.5 (SEQ ID NO: 15694); ENST00000543049.1 (SEQ ID NO: 13002); |
| | ENST00000543103.1 (SEQ ID NO: 13004); ENST00000543141.5 (SEQ ID NO: 13006); |
| | ENST00000543402.1 (SEQ ID NO: 13008); ENST00000544189.1 (SEQ ID NO: 13010); |
| | ENST00000544641.5 (SEQ ID NO: 13012); ENST00000545081.5 (SEQ ID NO: 15695); |
| | ENST00000545349.1; (SEQ ID NO: 13015) |
| transcript | ENST00000262510.10 (SEQ ID NO: 12971); ENST00000399221.3 (SEQ ID NO: 12973); |
| | ENST00000436936.5 (SEQ ID NO: 12974); ENST00000534927.5 (SEQ ID NO: 12976); |
| | ENST00000534931.1 (SEQ ID NO: 12978); ENST00000535284.1 (SEQ ID NO: 12980); |
| | ENST00000535658.1 (SEQ ID NO: 12982); ENST00000536231.1 (SEQ ID NO: 12984); |
| | ENST00000537056.5 (SEQ ID NO: 12985); ENST00000538059.5 (SEQ ID NO: 12987); |
| | ENST00000538110.5 (SEQ ID NO: 12988); ENST00000538273.5 (SEQ ID NO: 12989); |
| | ENST00000538453.5 (SEQ ID NO: 12990); ENST00000538778.5 (SEQ ID NO: 12992); |
| | ENST00000538805.5 (SEQ ID NO: 12993); ENST00000538930.5 (SEQ ID NO: 12995); |
| | ENST00000539144.5 (SEQ ID NO: 12996); ENST00000539881.5 (SEQ ID NO: 12997); |
| | ENST00000540182.5 (SEQ ID NO: 12998); ENST00000541020.1 (SEQ ID NO: 13000); |
| | ENST00000543030.5 (SEQ ID NO: 13001); ENST00000543049.1 (SEQ ID NO: 13003); |
| | ENST00000543103.1 (SEQ ID NO: 13005); ENST00000543141.5 (SEQ ID NO: 13007); |
| | ENST00000543402.1 (SEQ ID NO: 13009); ENST00000544189.1 (SEQ ID NO: 13011); |
| | ENST00000544641.5 (SEQ ID NO: 13013); ENST00000545081.5 (SEQ ID NO: 13014); |
| | ENST00000545349.1 (SEQ ID NO: 13016) |

Target: NOM1

| | |
|---|---|
| pre-mRNA | ENST00000275820.4 (SEQ ID NO: 13017); ENST00000460332.1 (SEQ ID NO: 13019); |
| transcript | ENST00000469271.1 (SEQ ID NO: 13021); ENST00000472491.1 (SEQ ID NO: 13023); |
| | ENST00000475176.1 (SEQ ID NO: 13025); ENST00000485661.1 (SEQ ID NO: 13027); |
| | ENST00000486131.1 (SEQ ID NO: 13029); ENST00000489850.5 (SEQ ID NO: 13031) |
| transcript | ENST00000275820.4 (SEQ ID NO: 13018); ENST00000460332.1 (SEQ ID NO: 13020); |
| | ENST00000469271.1 (SEQ ID NO: 13022); ENST00000472491.1 (SEQ ID NO: 13024); |
| | ENST00000475176.1 (SEQ ID NO: 13026); ENST00000485661.1 (SEQ ID NO: 13028); |
| | ENST00000486131.1 (SEQ ID NO: 13030); ENST00000489850.5 (SEQ ID NO: 13032) |

Target: NOP58

| | |
|---|---|
| pre-mRNA | ENST00000264279.10 (SEQ ID NO: 15696); ENST00000426814.5 (SEQ ID NO: 13034); |
| transcript | ENST00000433543.2 (SEQ ID NO: 13036); ENST00000467734.5 (SEQ ID NO: 13038); |
| | ENST00000472050.5 (SEQ ID NO: 13040); ENST00000478508.1 (SEQ ID NO: 13042); |
| | ENST00000478941.1 (SEQ ID NO: 13044); ENST00000488403.5 (SEQ ID NO: 13046); |
| | ENST00000492688.5 (SEQ ID NO: 13048); ENST00000492740.5 (SEQ ID NO: 13050) |
| transcript | ENST00000264279.10 (SEQ ID NO: 13033); ENST00000426814.5 (SEQ ID NO: 13035); |
| | ENST00000433543.2 (SEQ ID NO: 13037); ENST00000467734.5 (SEQ ID NO: 13039); |
| | ENST00000472050.5 (SEQ ID NO: 13041); ENST00000478508.1 (SEQ ID NO: 13043); |
| | ENST00000478941.1 (SEQ ID NO: 13045); ENST00000488403.5 (SEQ ID NO: 13047); |
| | ENST00000492688.5 (SEQ ID NO: 13049); ENST00000492740.5 (SEQ ID NO: 13051) |

Target: NPHP1

| | |
|---|---|
| pre-mRNA | ENST00000316534.8 (SEQ ID NO: 15697); ENST00000355301.8 (SEQ ID NO: 15698); |
| transcript | ENST00000393272.7 (SEQ ID NO: 15699); ENST00000417665.5 (SEQ ID NO: 15700); |
| | ENST00000418527.1 (SEQ ID NO: 13056); ENST00000422492.1 (SEQ ID NO: 13058); |
| | ENST00000445609.6 (SEQ ID NO: 15701); ENST00000449600.1 (SEQ ID NO: 13061); |
| | ENST00000461707.5 (SEQ ID NO: 15702); ENST00000493051.1 (SEQ ID NO: 13064); |
| | ENST00000496524.5 (SEQ ID NO: 15703) |
| transcript | ENST00000316534.8 (SEQ ID NO: 13052); ENST00000355301.8 (SEQ ID NO: 13053); |
| | ENST00000393272.7 (SEQ ID NO: 13054); ENST00000417665.5 (SEQ ID NO: 13055); |
| | ENST00000418527.1 (SEQ ID NO: 13057); ENST00000422492.1 (SEQ ID NO: 13059); |
| | ENST00000445609.6 (SEQ ID NO: 13060); ENST00000449600.1 (SEQ ID NO: 13062); |
| | ENST00000461707.5 (SEQ ID NO: 13063); ENST00000493051.1 (SEQ ID NO: 13065); |
| | ENST00000496524.5 (SEQ ID NO: 13066) |

Target: NPR1

| | |
|---|---|
| pre-mRNA | ENST00000368677.2 (SEQ ID NO: 13067); ENST00000368680.4 (SEQ ID NO: 13069); |
| transcript | ENST00000413826.1 (SEQ ID NO: 13071) |
| transcript | ENST00000368677.2 (SEQ ID NO: 13068); ENST00000368680.4 (SEQ ID NO: 13070); |
| | ENST00000413826.1 (SEQ ID NO: 13072) |

Target: NUP188

| | |
|---|---|
| pre-mRNA | ENST00000372577.2 (SEQ ID NO: 15704); ENST00000464729.1 (SEQ ID NO: 13074); |
| transcript | ENST00000465344.1 (SEQ ID NO: 13076); ENST00000467044.1 (SEQ ID NO: 13078); |
| | ENST00000477069.5 (SEQ ID NO: 13080); ENST00000485158.1 (SEQ ID NO: 13082); |
| | ENST00000487952.1 (SEQ ID NO: 13084); ENST00000491502.1 (SEQ ID NO: 13086); |
| | ENST00000491990.5 (SEQ ID NO: 13088); ENST00000495726.1 (SEQ ID NO: 13090); |
| | ENST00000550219.1 (SEQ ID NO: 13092) |
| transcript | ENST00000372577.2 (SEQ ID NO: 13073); ENST00000464729.1 (SEQ ID NO: 13075); |
| | ENST00000465344.1 (SEQ ID NO: 13077); ENST00000467044.1 (SEQ ID NO: 13079); |
| | ENST00000477069.5 (SEQ ID NO: 13081); ENST00000485158.1 (SEQ ID NO: 13083); |
| | ENST00000487952.1 (SEQ ID NO: 13085); ENST00000491502.1 (SEQ ID NO: 13087); |
| | ENST00000491990.5 (SEQ ID NO: 13089); ENST00000495726.1 (SEQ ID NO: 13091); |
| | ENST00000550219.1 (SEQ ID NO: 13093) |

Target: OSGEP

| | |
|---|---|
| pre-mRNA | ENST00000206542.9 (SEQ ID NO: 13094); ENST00000553292.1 (SEQ ID NO: 13096); |
| transcript | ENST00000553640.3 (SEQ ID NO: 13098); ENST00000554249.5 (SEQ ID NO: 13100); |
| | ENST00000554699.1 (SEQ ID NO: 13102); ENST00000554915.1 (SEQ ID NO: 13104); |
| | ENST00000555223.5 (SEQ ID NO: 13106); ENST00000555656.5 (SEQ ID NO: 13108); |
| | ENST00000555785.2 (SEQ ID NO: 13110); ENST00000556124.3 (SEQ ID NO: 13112); |
| | ENST00000556252.1 (SEQ ID NO: 13114); ENST00000556439.1; (SEQ ID NO: 13116) |
| transcript | ENST00000206542.9 (SEQ ID NO: 13095); ENST00000553292.1 (SEQ ID NO: 13097); |
| | ENST00000553640.3 (SEQ ID NO: 13099); ENST00000554249.5 (SEQ ID NO: 13101); |
| | ENST00000554699.1 (SEQ ID NO: 13103); ENST00000554915.1 (SEQ ID NO: 13105); |
| | ENST00000555223.5 (SEQ ID NO: 13107); ENST00000555656.5 (SEQ ID NO: 13109); |
| | ENST00000555785.2 (SEQ ID NO: 13111); ENST00000556124.3 (SEQ ID NO: 13113); |
| | ENST00000556252.1 (SEQ ID NO: 13115); ENST00000556439.1 (SEQ ID NO: 13117) |

Target: PABPC4

| | |
|---|---|
| pre-mRNA | ENST00000372856.7 (SEQ ID NO: 13118); ENST00000372857.7 (SEQ ID NO: 13120); |
| transcript | ENST00000372858.8 (SEQ ID NO: 13122); ENST00000372862.7 (SEQ ID NO: 13124); |
| | ENST00000421687.6 (SEQ ID NO: 13126); ENST00000437136.5 (SEQ ID NO: 13128); |
| | ENST00000451091.2 (SEQ ID NO: 13130); ENST00000461578.1 (SEQ ID NO: 13132); |
| | ENST00000468476.1 (SEQ ID NO: 13134); ENST00000470443.5 (SEQ ID NO: 13136); |
| | ENST00000474378.1 (SEQ ID NO: 13138); ENST00000477556.1 (SEQ ID NO: 13140); |
| | ENST00000482028.5 (SEQ ID NO: 13142); ENST00000483770.1 (SEQ ID NO: 13144); |
| | ENST00000484555.1 (SEQ ID NO: 13146); ENST00000492468.5 (SEQ ID NO: 13148); |
| | ENST00000492519.1 (SEQ ID NO: 13150); ENST00000513632.2 (SEQ ID NO: 13152); |
| | ENST00000525045.5 (SEQ ID NO: 13154); ENST00000525669.5 (SEQ ID NO: 13156); |
| | ENST00000525751.5 (SEQ ID NO: 13158); ENST00000527718.1 (SEQ ID NO: 13160); |
| | ENST00000529216.1 (SEQ ID NO: 13162); ENST00000530186.1 (SEQ ID NO: 13164); |
| | ENST00000531243.2 (SEQ ID NO: 13166) |
| transcript | ENST00000372856.7 (SEQ ID NO: 13119); ENST00000372857.7 (SEQ ID NO: 13121); |
| | ENST00000372858.8 (SEQ ID NO: 13123); ENST00000372862.7 (SEQ ID NO: 13125); |
| | ENST00000421687.6 (SEQ ID NO: 13127); ENST00000437136.5 (SEQ ID NO: 13129); |
| | ENST00000451091.2 (SEQ ID NO: 13131); ENST00000461578.1 (SEQ ID NO: 13133); |
| | ENST00000468476.1 (SEQ ID NO: 13135); ENST00000470443.5 (SEQ ID NO: 13137); |
| | ENST00000474378.1 (SEQ ID NO: 13139); ENST00000477556.1 (SEQ ID NO: 13141); |
| | ENST00000482028.5 (SEQ ID NO: 13143); ENST00000483770.1 (SEQ ID NO: 13145); |
| | ENST00000484555.1 (SEQ ID NO: 13147); ENST00000492468.5 (SEQ ID NO: 13149); |
| | ENST00000492519.1 (SEQ ID NO: 13151); ENST00000513632.2 (SEQ ID NO: 13153); |
| | ENST00000525045.5 (SEQ ID NO: 13155); ENST00000525669.5 (SEQ ID NO: 13157); |
| | ENST00000525751.5 (SEQ ID NO: 13159); ENST00000527718.1 (SEQ ID NO: 13161); |
| | ENST00000529216.1 (SEQ ID NO: 13163); ENST00000530186.1 (SEQ ID NO: 13165); |
| | ENST00000531243.2 (SEQ ID NO: 13167) |

Target: PCOLCE

| | |
|---|---|
| pre-mRNA | ENST00000223061.6 (SEQ ID NO: 13168); ENST00000460002.1 (SEQ ID NO: 13170); |
| transcript | ENST00000462260.1 (SEQ ID NO: 13172); ENST00000468214.1 (SEQ ID NO: 13174); |
| | ENST00000472348.1 (SEQ ID NO: 13176); ENST00000482863.1 (SEQ ID NO: 13178); |
| | ENST00000486440.5 (SEQ ID NO: 13180); ENST00000487172.1 (SEQ ID NO: 13182); |
| | ENST00000490909.1 (SEQ ID NO: 13184); ENST00000496269.1 (SEQ ID NO: 13186) |
| transcript | ENST00000223061.6 (SEQ ID NO: 13169); ENST00000460002.1 (SEQ ID NO: 13171); |
| | ENST00000462260.1 (SEQ ID NO: 13173); ENST00000468214.1 (SEQ ID NO: 13175); |
| | ENST00000472348.1 (SEQ ID NO: 13177); ENST00000482863.1 (SEQ ID NO: 13179); |
| | ENST00000486440.5 (SEQ ID NO: 13181); ENST00000487172.1 (SEQ ID NO: 13183); |
| | ENST00000490909.1 (SEQ ID NO: 13185); ENST00000496269.1 (SEQ ID NO: 13187) |

Target: PHKA2

| | |
|---|---|
| pre-mRNA | ENST00000379942.5 (SEQ ID NO: 15705); ENST00000464455.1 (SEQ ID NO: 13189); |
| transcript | ENST00000469485.5 (SEQ ID NO: 13191); ENST00000469645.5 (SEQ ID NO: 13193); |
| | ENST00000473597.1 (SEQ ID NO: 13195); ENST00000473739.5 (SEQ ID NO: 13197); |
| | ENST00000481718.1 (SEQ ID NO: 13199); ENST00000486231.2 (SEQ ID NO: 13201) |
| transcript | ENST00000379942.5 (SEQ ID NO: 13188); ENST00000464455.1 (SEQ ID NO: 13190); |
| | ENST00000469485.5 (SEQ ID NO: 13192); ENST00000469645.5 (SEQ ID NO: 13194); |
| | ENST00000473597.1 (SEQ ID NO: 13196); ENST00000473739.5 (SEQ ID NO: 13198); |
| | ENST00000481718.1 (SEQ ID NO: 13200); ENST00000486231.2 (SEQ ID NO: 13202) |

Target: PIDD1

| | |
|---|---|
| pre-mRNA | ENST00000347755.10 (SEQ ID NO: 13203); ENST00000411829.6 (SEQ ID NO: 13205); |
| transcript | ENST00000524486.5 (SEQ ID NO: 13207); ENST00000525028.6 (SEQ ID NO: 13209); |
| | ENST00000527357.5 (SEQ ID NO: 13211); ENST00000527812.1 (SEQ ID NO: 13213); |
| | ENST00000528122.1 (SEQ ID NO: 13215); ENST00000530911.1 (SEQ ID NO: 13217); |
| | ENST00000531286.5 (SEQ ID NO: 13219); ENST00000534525.5 (SEQ ID NO: 13221); |
| | ENST00000534649.1 (SEQ ID NO: 13223) |
| transcript | ENST00000347755.10 (SEQ ID NO: 13204); ENST00000411829.6 (SEQ ID NO: 13206); |
| | ENST00000524486.5 (SEQ ID NO: 13208); ENST00000525028.6 (SEQ ID NO: 13210); |
| | ENST00000527357.5 (SEQ ID NO: 13212); ENST00000527812.1 (SEQ ID NO: 13214); |
| | ENST00000528122.1 (SEQ ID NO: 13216); ENST00000530911.1 (SEQ ID NO: 13218); |
| | ENST00000531286.5 (SEQ ID NO: 13220); ENST00000534525.5 (SEQ ID NO: 13222); |
| | ENST00000534649.1 (SEQ ID NO: 13224) |

Target: PKD1

| | |
|---|---|
| pre-mRNA | ENST00000262304.9 (SEQ ID NO: 15706); ENST00000415938.7 (SEQ ID NO: 13226); |
| transcript | ENST00000423118.5 (SEQ ID NO: 15707); ENST00000468674.5 (SEQ ID NO: 13229); |
| | ENST00000469241.2 (SEQ ID NO: 13231); ENST00000469851.1 (SEQ ID NO: 13233); |
| | ENST00000471603.6 (SEQ ID NO: 13235); ENST00000472577.1 (SEQ ID NO: 13237); |
| | ENST00000472659.1 (SEQ ID NO: 13239); ENST00000473780.2 (SEQ ID NO: 13241); |
| | ENST00000474088.1 (SEQ ID NO: 13243); ENST00000475889.1 (SEQ ID NO: 13245); |
| | ENST00000480227.5 (SEQ ID NO: 13247); ENST00000483024.1 (SEQ ID NO: 13249); |
| | ENST00000483558.5 (SEQ ID NO: 13251); ENST00000483731.5 (SEQ ID NO: 13253); |
| | ENST00000483814.1 (SEQ ID NO: 13255); ENST00000485120.1 (SEQ ID NO: 13257); |
| | ENST00000486339.6 (SEQ ID NO: 13259); ENST00000487932.5 (SEQ ID NO: 13261); |
| | ENST00000488185.2 (SEQ ID NO: 13263); ENST00000496574.6 (SEQ ID NO: 13265); |
| | ENST00000561668.5 (SEQ ID NO: 13267); ENST00000561991.5 (SEQ ID NO: 13269); |
| | ENST00000562297.5 (SEQ ID NO: 13271); ENST00000562425.1 (SEQ ID NO: 13273); |
| | ENST00000564313.1 (SEQ ID NO: 13275); ENST00000564865.5 (SEQ ID NO: 13277); |
| | ENST00000564890.1 (SEQ ID NO: 13279); ENST00000565639.6 (SEQ ID NO: 13281); |
| | ENST00000566784.5 (SEQ ID NO: 13283); ENST00000566905.5 (SEQ ID NO: 13285); |
| | ENST00000567355.1 (SEQ ID NO: 13287); ENST00000567946.1 (SEQ ID NO: 13289); |
| | ENST00000568591.5 (SEQ ID NO: 13291); ENST00000568796.1 (SEQ ID NO: 13293); |
| | ENST00000569983.5 (SEQ ID NO: 13295); ENST00000570150.1 (SEQ ID NO: 13297); |
| | ENST00000570193.5 (SEQ ID NO: 13299); ENST00000570253.5 (SEQ ID NO: 13301) |
| transcript | ENST00000262304.9 (SEQ ID NO: 13225); ENST00000415938.7 (SEQ ID NO: 13227); |
| | ENST00000423118.5 (SEQ ID NO: 13228); ENST00000468674.5 (SEQ ID NO: 13230); |
| | ENST00000469241.2 (SEQ ID NO: 13232); ENST00000469851.1 (SEQ ID NO: 13234); |
| | ENST00000471603.6 (SEQ ID NO: 13236); ENST00000472577.1 (SEQ ID NO: 13238); |
| | ENST00000472659.1 (SEQ ID NO: 13240); ENST00000473780.2 (SEQ ID NO: 13242); |
| | ENST00000474088.1 (SEQ ID NO: 13244); ENST00000475889.1 (SEQ ID NO: 13246); |
| | ENST00000480227.5 (SEQ ID NO: 13248); ENST00000483024.1 (SEQ ID NO: 13250); |
| | ENST00000483558.5 (SEQ ID NO: 13252); ENST00000483731.5 (SEQ ID NO: 13254); |
| | ENST00000483814.1 (SEQ ID NO: 13256); ENST00000485120.1 (SEQ ID NO: 13258); |
| | ENST00000486339.6 (SEQ ID NO: 13260); ENST00000487932.5 (SEQ ID NO: 13262); |
| | ENST00000488185.2 (SEQ ID NO: 13264); ENST00000496574.6 (SEQ ID NO: 13266); |
| | ENST00000561668.5 (SEQ ID NO: 13268); ENST00000561991.5 (SEQ ID NO: 13270); |
| | ENST00000562297.5 (SEQ ID NO: 13272); ENST00000562425.1 (SEQ ID NO: 13274); |
| | ENST00000564313.1 (SEQ ID NO: 13276); ENST00000564865.5 (SEQ ID NO: 13278); |
| | ENST00000564890.1 (SEQ ID NO: 13280); ENST00000565639.6 (SEQ ID NO: 13282); |
| | ENST00000566784.5 (SEQ ID NO: 13284); ENST00000566905.5 (SEQ ID NO: 13286); |
| | ENST00000567355.1 (SEQ ID NO: 13288); ENST00000567946.1 (SEQ ID NO: 13290); |
| | ENST00000568591.5 (SEQ ID NO: 13292); ENST00000568796.1 (SEQ ID NO: 13294); |
| | ENST00000569983.5 (SEQ ID NO: 13296); ENST00000570150.1 (SEQ ID NO: 13298); |
| | ENST00000570193.5 (SEQ ID NO: 13300); ENST00000570253.5 (SEQ ID NO: 13302) |

Target: PLA2G6

| | |
|---|---|
| pre-mRNA | ENST00000332509.8 (SEQ ID NO: 15708); ENST00000335539.7 (SEQ ID NO: 15709); |
| transcript | ENST00000402064.5 (SEQ ID NO: 15710); ENST00000417303.6 (SEQ ID NO: 15711); |
| | ENST00000420435.5 (SEQ ID NO: 15712); ENST00000426674.1 (SEQ ID NO: 15713); |
| | ENST00000427114.6 (SEQ ID NO: 15714); ENST00000427453.5 (SEQ ID NO: 13310); |
| | ENST00000430886.5 (SEQ ID NO: 15715); ENST00000435484.5 (SEQ ID NO: 15716); |
| | ENST00000436218.6 (SEQ ID NO: 15717); ENST00000445591.5 (SEQ ID NO: 13315); |
| | ENST00000447598.6 (SEQ ID NO: 15718); ENST00000448094.5 (SEQ ID NO: 13318); |
| | ENST00000452542.5 (SEQ ID NO: 13320); ENST00000452794.5 (SEQ ID NO: 13322); |
| | ENST00000452972.1 (SEQ ID NO: 13324); ENST00000454670.1 (SEQ ID NO: 13326); |
| | ENST00000455341.2 (SEQ ID NO: 15719); ENST00000463287.1 (SEQ ID NO: 13329); |
| | ENST00000471636.5 (SEQ ID NO: 15720); ENST00000479641.5 (SEQ ID NO: 13332); |
| | ENST00000480154.1 (SEQ ID NO: 13334); ENST00000490473.1 (SEQ ID NO: 13336); |
| | ENST00000491986.1 (SEQ ID NO: 13338); ENST00000496409.1 (SEQ ID NO: 13340); |
| | ENST00000498338.1 (SEQ ID NO: 13342); ENST00000594306.1 (SEQ ID NO: 15721); |
| | ENST00000655142.1 (SEQ ID NO: 15722); ENST00000660610.1 (SEQ ID NO: 15723); |
| | ENST00000663895.1 (SEQ ID NO: 15724); ENST00000664587.1 (SEQ ID NO: 15725); |
| | ENST00000665987.1 (SEQ ID NO: 15726); ENST00000667521.1 (SEQ ID NO: 15727); |
| | ENST00000668208.1 (SEQ ID NO: 15728); ENST00000668499.1 (SEQ ID NO: 15729); |
| | ENST00000668949.1 (SEQ ID NO: 15730); ENST00000671093.1 (SEQ ID NO: 15731); |
| | ENST00000673413.1 (SEQ ID NO: 15732) |
| transcript | ENST00000332509.8 (SEQ ID NO: 13303); ENST00000335539.7 (SEQ ID NO: 13304); |
| | ENST00000402064.5 (SEQ ID NO: 13305); ENST00000417303.6 (SEQ ID NO: 13306); |
| | ENST00000420435.5 (SEQ ID NO: 13307); ENST00000426674.1 (SEQ ID NO: 13308); |
| | ENST00000427114.6 (SEQ ID NO: 13309); ENST00000427453.5 (SEQ ID NO: 13311); |
| | ENST00000430886.5 (SEQ ID NO: 13312); ENST00000435484.5 (SEQ ID NO: 13313); |
| | ENST00000436218.6 (SEQ ID NO: 13314); ENST00000445591.5 (SEQ ID NO: 13316); |
| | ENST00000447598.6 (SEQ ID NO: 13317); ENST00000448094.5 (SEQ ID NO: 13319); |
| | ENST00000452542.5 (SEQ ID NO: 13321); ENST00000452794.5 (SEQ ID NO: 13323); |
| | ENST00000452972.1 (SEQ ID NO: 13325); ENST00000454670.1 (SEQ ID NO: 13327); |
| | ENST00000455341.2 (SEQ ID NO: 13328); ENST00000463287.1 (SEQ ID NO: 13330); |
| | ENST00000471636.5 (SEQ ID NO: 13331); ENST00000479641.5 (SEQ ID NO: 13333); |
| | ENST00000480154.1 (SEQ ID NO: 13335); ENST00000490473.1 (SEQ ID NO: 13337); |
| | ENST00000491986.1 (SEQ ID NO: 13339); ENST00000496409.1 (SEQ ID NO: 13341); |
| | ENST00000498338.1 (SEQ ID NO: 13343); ENST00000594306.1 (SEQ ID NO: 13344); |
| | ENST00000655142.1 (SEQ ID NO: 13345); ENST00000660610.1 (SEQ ID NO: 13346); |
| | ENST00000663895.1 (SEQ ID NO: 13347); ENST00000664587.1 (SEQ ID NO: 13348); |
| | ENST00000665987.1 (SEQ ID NO: 13349); ENST00000667521.1 (SEQ ID NO: 13350); |
| | ENST00000668208.1 (SEQ ID NO: 13351); ENST00000668499.1 (SEQ ID NO: 13352); |
| | ENST00000668949.1 (SEQ ID NO: 13353); ENST00000671093.1 (SEQ ID NO: 13354); |
| | ENST00000673413.1 (SEQ ID NO: 13355) |

Target: PLD2

| | |
|---|---|
| pre-mRNA | ENST00000263088.11 (SEQ ID NO: 13356); ENST00000571273.5 (SEQ ID NO: 13358); |
| transcript | ENST00000572127.1 (SEQ ID NO: 13360); ENST00000572199.1 (SEQ ID NO: 13362); |
| | ENST00000572940.5 (SEQ ID NO: 13364); ENST00000573258.1 (SEQ ID NO: 13366); |
| | ENST00000574268.1 (SEQ ID NO: 13368); ENST00000574796.1 (SEQ ID NO: 13370); |
| | ENST00000575246.6 (SEQ ID NO: 13372); ENST00000575316.1 (SEQ ID NO: 13374); |
| | ENST00000575813.5 (SEQ ID NO: 13376); ENST00000575945.1 (SEQ ID NO: 13378); |
| | ENST00000576329.1 (SEQ ID NO: 13380); ENST00000576864.1 (SEQ ID NO: 13382); |
| | ENST00000576983.5 (SEQ ID NO: 13384) |
| transcript | ENST00000263088.11 (SEQ ID NO: 13357); ENST00000571273.5 (SEQ ID NO: 13359); |
| | ENST00000572127.1 (SEQ ID NO: 13361); ENST00000572199.1 (SEQ ID NO: 13363); |
| | ENST00000572940.5 (SEQ ID NO: 13365); ENST00000573258.1 (SEQ ID NO: 13367); |
| | ENST00000574268.1 (SEQ ID NO: 13369); ENST00000574796.1 (SEQ ID NO: 13371); |
| | ENST00000575246.6 (SEQ ID NO: 13373); ENST00000575316.1 (SEQ ID NO: 13375); |
| | ENST00000575813.5 (SEQ ID NO: 13377); ENST00000575945.1 (SEQ ID NO: 13379); |
| | ENST00000576329.1 (SEQ ID NO: 13381); ENST00000576864.1 (SEQ ID NO: 13383); |
| | ENST00000576983.5 (SEQ ID NO: 13385) |

Target: PLEKHG5

| | |
|---|---|
| pre-mRNA | ENST00000340850.9 (SEQ ID NO: 13386); ENST00000377725.5 (SEQ ID NO: 13388); |
| transcript | ENST00000377728.7 (SEQ ID NO: 13390); ENST00000377732.5 (SEQ ID NO: 13392); |
| | ENST00000377740.4 (SEQ ID NO: 15733); ENST00000377748.5 (SEQ ID NO: 15734); |
| | ENST00000400913.5 (SEQ ID NO: 13396); ENST00000400915.7 (SEQ ID NO: 15735); |
| | ENST00000487949.4 (SEQ ID NO: 13399); ENST00000489097.5 (SEQ ID NO: 13401); |
| | ENST00000535355.5 (SEQ ID NO: 13403); ENST00000537245.5 (SEQ ID NO: 15736); |
| | ENST00000673471.1 (SEQ ID NO: 15737) |
| transcript | ENST00000340850.9 (SEQ ID NO: 13387); ENST00000377725.5 (SEQ ID NO: 13389); |
| | ENST00000377728.7 (SEQ ID NO: 13391); ENST00000377732.5 (SEQ ID NO: 13393); |
| | ENST00000377740.4 (SEQ ID NO: 13394); ENST00000377748.5 (SEQ ID NO: 13395); |
| | ENST00000400913.5 (SEQ ID NO: 13397); ENST00000400915.7 (SEQ ID NO: 13398); |
| | ENST00000487949.4 (SEQ ID NO: 13400); ENST00000489097.5 (SEQ ID NO: 13402); |
| | ENST00000535355.5 (SEQ ID NO: 13404); ENST00000537245.5 (SEQ ID NO: 13405); |
| | ENST00000673471.1 (SEQ ID NO: 13406) |

Target: PNPO

| | |
|---|---|
| pre-mRNA | ENST00000225573.5 (SEQ ID NO: 13407); ENST00000434554.7 (SEQ ID NO: 13409); |
| transcript | ENST00000582171.6 (SEQ ID NO: 13411); ENST00000583245.6 (SEQ ID NO: 13413); |
| | ENST00000583599.6 (SEQ ID NO: 13415); ENST00000584061.6 (SEQ ID NO: 13417); |
| | ENST00000584806.2 (SEQ ID NO: 13419); ENST00000585320.5 (SEQ ID NO: 13421); |
| | ENST00000641285.1 (SEQ ID NO: 13423); ENST00000641305.1 (SEQ ID NO: 13425); |
| | ENST00000641323.1 (SEQ ID NO: 13427); ENST00000641427.1 (SEQ ID NO: 13429); |
| | ENST00000641511.1 (SEQ ID NO: 13431); ENST00000641703.1 (SEQ ID NO: 13433); |
| | ENST00000641709.1 (SEQ ID NO: 13435); ENST00000641856.1 (SEQ ID NO: 13437); |
| | ENST00000642017.1; (SEQ ID NO: 13439) |
| transcript | ENST00000225573.5 (SEQ ID NO: 13408); ENST00000434554.7 (SEQ ID NO: 13410); |
| | ENST00000582171.6 (SEQ ID NO: 13412); ENST00000583245.6 (SEQ ID NO: 13414); |
| | ENST00000583599.6 (SEQ ID NO: 13416); ENST00000584061.6 (SEQ ID NO: 13418); |
| | ENST00000584806.2 (SEQ ID NO: 13420); ENST00000585320.5 (SEQ ID NO: 13422); |
| | ENST00000641285.1 (SEQ ID NO: 13424); ENST00000641305.1 (SEQ ID NO: 13426); |
| | ENST00000641323.1 (SEQ ID NO: 13428); ENST00000641427.1 (SEQ ID NO: 13430); |
| | ENST00000641511.1 (SEQ ID NO: 13432); ENST00000641703.1 (SEQ ID NO: 13434); |
| | ENST00000641709.1 (SEQ ID NO: 13436); ENST00000641856.1 (SEQ ID NO: 13438); |
| | ENST00000642017.1 (SEQ ID NO: 13440) |

Target: POLE

| | |
|---|---|
| pre-mRNA | ENST00000320574.10 (SEQ ID NO: 15738); ENST00000416953.2 (SEQ ID NO: 13442); |
| transcript | ENST00000434528.4 (SEQ ID NO: 13444); ENST00000441786.3 (SEQ ID NO: 13446); |
| | ENST00000503265.4 (SEQ ID NO: 13448); ENST00000534922.5 (SEQ ID NO: 13450); |
| | ENST00000535270.5 (SEQ ID NO: 15739); ENST00000535934.2 (SEQ ID NO: 13453); |
| | ENST00000536445.5 (SEQ ID NO: 13455); ENST00000537064.5 (SEQ ID NO: 15740); |
| | ENST00000538196.1 (SEQ ID NO: 13458); ENST00000539215.5 (SEQ ID NO: 13460); |
| | ENST00000539357.1 (SEQ ID NO: 13462); ENST00000539618.1 (SEQ ID NO: 13464); |
| | ENST00000540987.1 (SEQ ID NO: 13466); ENST00000541213.5 (SEQ ID NO: 13468); |
| | ENST00000541627.2 (SEQ ID NO: 13470); ENST00000544414.1 (SEQ ID NO: 13472); |
| | ENST00000544692.5 (SEQ ID NO: 13474); ENST00000544870.5 (SEQ ID NO: 13476); |
| | ENST00000545015.1 (SEQ ID NO: 13478); ENST00000672002.1 (SEQ ID NO: 15741); |
| | ENST00000672742.1; (SEQ ID NO: 15742) |
| transcript | ENST00000320574.10 (SEQ ID NO: 13441); ENST00000416953.2 (SEQ ID NO: 13443); |
| | ENST00000434528.4 (SEQ ID NO: 13445); ENST00000441786.3 (SEQ ID NO: 13447); |
| | ENST00000503265.4 (SEQ ID NO: 13449); ENST00000534922.5 (SEQ ID NO: 13451); |
| | ENST00000535270.5 (SEQ ID NO: 13452); ENST00000535934.2 (SEQ ID NO: 13454); |
| | ENST00000536445.5 (SEQ ID NO: 13456); ENST00000537064.5 (SEQ ID NO: 13457); |
| | ENST00000538196.1 (SEQ ID NO: 13459); ENST00000539215.5 (SEQ ID NO: 13461); |
| | ENST00000539357.1 (SEQ ID NO: 13463); ENST00000539618.1 (SEQ ID NO: 13465); |
| | ENST00000540987.1 (SEQ ID NO: 13467); ENST00000541213.5 (SEQ ID NO: 13469); |
| | ENST00000541627.2 (SEQ ID NO: 13471); ENST00000544414.1 (SEQ ID NO: 13473); |
| | ENST00000544692.5 (SEQ ID NO: 13475); ENST00000544870.5 (SEQ ID NO: 13477); |
| | ENST00000545015.1 (SEQ ID NO: 13479); ENST00000672002.1 (SEQ ID NO: 13480); |
| | ENST00000672742.1 (SEQ ID NO: 13481) |

Target: PON2

| | |
|---|---|
| pre-mRNA | ENST00000222572.8 (SEQ ID NO: 15743); ENST00000433091.6 (SEQ ID NO: 15744); |
| transcript | ENST00000446142.5 (SEQ ID NO: 13484); ENST00000455123.5 (SEQ ID NO: 15745); |
| | ENST00000459842.1 (SEQ ID NO: 13487); ENST00000460873.5 (SEQ ID NO: 13489); |
| | ENST00000469716.1 (SEQ ID NO: 13491); ENST00000469926.5 (SEQ ID NO: 13493); |
| | ENST00000471883.1 (SEQ ID NO: 13495); ENST00000478801.5 (SEQ ID NO: 13497); |
| | ENST00000483292.5 (SEQ ID NO: 13499); ENST00000490778.5 (SEQ ID NO: 13501); |
| | ENST00000491069.5 (SEQ ID NO: 13503); ENST00000493290.5 (SEQ ID NO: 13505); |
| | ENST00000493469.5 (SEQ ID NO: 13507); ENST00000632034.1 (SEQ ID NO: 13509); |
| | ENST00000633192.1 (SEQ ID NO: 15746); ENST00000633531.1 (SEQ ID NO: 15747) |
| transcript | ENST00000222572.8 (SEQ ID NO: 13482); ENST00000433091.6 (SEQ ID NO: 13483); |
| | ENST00000446142.5 (SEQ ID NO: 13485); ENST00000455123.5 (SEQ ID NO: 13486); |
| | ENST00000459842.1 (SEQ ID NO: 13488); ENST00000460873.5 (SEQ ID NO: 13490); |
| | ENST00000469716.1 (SEQ ID NO: 13492); ENST00000469926.5 (SEQ ID NO: 13494); |
| | ENST00000471883.1 (SEQ ID NO: 13496); ENST00000478801.5 (SEQ ID NO: 13498); |
| | ENST00000483292.5 (SEQ ID NO: 13500); ENST00000490778.5 (SEQ ID NO: 13502); |
| | ENST00000491069.5 (SEQ ID NO: 13504); ENST00000493290.5 (SEQ ID NO: 13506); |
| | ENST00000493469.5 (SEQ ID NO: 13508); ENST00000632034.1 (SEQ ID NO: 13510); |
| | ENST00000633192.1 (SEQ ID NO: 13511); ENST00000633531.1 (SEQ ID NO: 13512) |

Target: PRMT7

| | |
|---|---|
| pre-mRNA | ENST00000339507.9 (SEQ ID NO: 15748); ENST00000441236.2 (SEQ ID NO: 15749); |
| transcript | ENST00000449359.7 (SEQ ID NO: 15750); ENST00000561806.5 (SEQ ID NO: 13516); |
| | ENST00000562050.5 (SEQ ID NO: 13518); ENST00000562381.7 (SEQ ID NO: 13520); |
| | ENST00000562456.1 (SEQ ID NO: 13522); ENST00000563443.1 (SEQ ID NO: 13524); |
| | ENST00000563520.5 (SEQ ID NO: 13526); ENST00000563562.5 (SEQ ID NO: 13528); |
| | ENST00000563608.2 (SEQ ID NO: 13530); ENST00000564050.6 (SEQ ID NO: 15751); |
| | ENST00000564441.5 (SEQ ID NO: 13533); ENST00000565356.5 (SEQ ID NO: 15752); |
| | ENST00000565745.5 (SEQ ID NO: 13536); ENST00000565761.1 (SEQ ID NO: 13538); |
| | ENST00000565983.5 (SEQ ID NO: 13540); ENST00000566341.5 (SEQ ID NO: 13542); |
| | ENST00000566430.2 (SEQ ID NO: 13544); ENST00000566657.5 (SEQ ID NO: 13546); |
| | ENST00000566687.1 (SEQ ID NO: 13548); ENST00000566708.1 (SEQ ID NO: 13550); |
| | ENST00000567542.5 (SEQ ID NO: 15753); ENST00000568463.1 (SEQ ID NO: 13553); |
| | ENST00000568975.5 (SEQ ID NO: 15754); ENST00000569047.7 (SEQ ID NO: 13556); |
| | ENST00000569571.5 (SEQ ID NO: 13558) |
| transcript | ENST00000339507.9 (SEQ ID NO: 13513); ENST00000441236.2 (SEQ ID NO: 13514); |
| | ENST00000449359.7 (SEQ ID NO: 13515); ENST00000561806.5 (SEQ ID NO: 13517); |
| | ENST00000562050.5 (SEQ ID NO: 13519); ENST00000562381.7 (SEQ ID NO: 13521); |
| | ENST00000562456.1 (SEQ ID NO: 13523); ENST00000563443.1 (SEQ ID NO: 13525); |
| | ENST00000563520.5 (SEQ ID NO: 13527); ENST00000563562.5 (SEQ ID NO: 13529); |
| | ENST00000563608.2 (SEQ ID NO: 13531); ENST00000564050.6 (SEQ ID NO: 13532); |
| | ENST00000564441.5 (SEQ ID NO: 13534); ENST00000565356.5 (SEQ ID NO: 13535); |
| | ENST00000565745.5 (SEQ ID NO: 13537); ENST00000565761.1 (SEQ ID NO: 13539); |
| | ENST00000565983.5 (SEQ ID NO: 13541); ENST00000566341.5 (SEQ ID NO: 13543); |
| | ENST00000566430.2 (SEQ ID NO: 13545); ENST00000566657.5 (SEQ ID NO: 13547); |
| | ENST00000566687.1 (SEQ ID NO: 13549); ENST00000566708.1 (SEQ ID NO: 13551); |
| | ENST00000567542.5 (SEQ ID NO: 13552); ENST00000568463.1 (SEQ ID NO: 13554); |
| | ENST00000568975.5 (SEQ ID NO: 13555); ENST00000569047.7 (SEQ ID NO: 13557); |
| | ENST00000569571.5 (SEQ ID NO: 13559) |

Target: PRODH

| | |
|---|---|
| pre-mRNA | ENST00000313755.9 (SEQ ID NO: 13560); ENST00000334029.6 (SEQ ID NO: 13562); |
| transcript | ENST00000357068.10 (SEQ ID NO: 13564); ENST00000399694.1 (SEQ ID NO: 13566); |
| | ENST00000420436.5 (SEQ ID NO: 13568); ENST00000429300.5 (SEQ ID NO: 13570); |
| | ENST00000438924.5 (SEQ ID NO: 13572); ENST00000446371.1 (SEQ ID NO: 13574); |
| | ENST00000450579.1 (SEQ ID NO: 13576); ENST00000457083.1 (SEQ ID NO: 13578); |
| | ENST00000482858.5 (SEQ ID NO: 13580); ENST00000491604.5 (SEQ ID NO: 13582); |
| | ENST00000496625.1 (SEQ ID NO: 13584); ENST00000609229.1 (SEQ ID NO: 13586); |
| | ENST00000610940.4; (SEQ ID NO: 13588) |
| transcript | ENST00000313755.9 (SEQ ID NO: 13561); ENST00000334029.6 (SEQ ID NO: 13563); |
| | ENST00000357068.10 (SEQ ID NO: 13565); ENST00000399694.1 (SEQ ID NO: 13567); |
| | ENST00000420436.5 (SEQ ID NO: 13569); ENST00000429300.5 (SEQ ID NO: 13571); |
| | ENST00000438924.5 (SEQ ID NO: 13573); ENST00000446371.1 (SEQ ID NO: 13575); |
| | ENST00000450579.1 (SEQ ID NO: 13577); ENST00000457083.1 (SEQ ID NO: 13579); |
| | ENST00000482858.5 (SEQ ID NO: 13581); ENST00000491604.5 (SEQ ID NO: 13583); |
| | ENST00000496625.1 (SEQ ID NO: 13585); ENST00000609229.1 (SEQ ID NO: 13587); |
| | ENST00000610940.4 (SEQ ID NO: 13589) |

Target: PRPF3

| | |
|---|---|
| pre-mRNA | ENST00000324862.7 (SEQ ID NO: 15755); ENST00000467329.5 (SEQ ID NO: 13591); |
| transcript | ENST00000467514.1 (SEQ ID NO: 13593); ENST00000470824.1 (SEQ ID NO: 13595); |
| | ENST00000476970.1 (SEQ ID NO: 13597); ENST00000493553.1 (SEQ ID NO: 13599); |
| | ENST00000496202.5; (SEQ ID NO: 13601) |
| transcript | ENST00000324862.7 (SEQ ID NO: 13590); ENST00000467329.5 (SEQ ID NO: 13592); |
| | ENST00000467514.1 (SEQ ID NO: 13594); ENST00000470824.1 (SEQ ID NO: 13596); |
| | ENST00000476970.1 (SEQ ID NO: 13598); ENST00000493553.1 (SEQ ID NO: 13600); |
| | ENST00000496202.5 (SEQ ID NO: 13602) |

Target: PRPF4

| | |
|---|---|
| pre-mRNA | ENST00000374198.4 (SEQ ID NO: 13603); ENST00000374199.8 (SEQ ID NO: 13605); |
| transcript | ENST00000488937.1; (SEQ ID NO: 13607) |
| transcript | ENST00000374198.4 (SEQ ID NO: 13604); ENST00000374199.8 (SEQ ID NO: 13606); |
| | ENST00000488937.1 (SEQ ID NO: 13608) |

Target: PYCR1

| | |
|---|---|
| pre-mRNA | ENST00000329875.13 (SEQ ID NO: 13609); ENST00000337943.9 (SEQ ID NO: 13611); |
| transcript | ENST00000402252.6 (SEQ ID NO: 13613); ENST00000403172.8 (SEQ ID NO: 13615); |
| | ENST00000405481.8 (SEQ ID NO: 13617); ENST00000577624.5 (SEQ ID NO: 13619); |
| | ENST00000577756.5 (SEQ ID NO: 13621); ENST00000579366.5 (SEQ ID NO: 13623); |
| | ENST00000579698.5 (SEQ ID NO: 13625); ENST00000581271.5 (SEQ ID NO: 13627); |
| | ENST00000582198.5 (SEQ ID NO: 13629); ENST00000583564.5 (SEQ ID NO: 13631); |
| | ENST00000584848.5 (SEQ ID NO: 13633); ENST00000585215.5 (SEQ ID NO: 13635); |
| | ENST00000585244.1 (SEQ ID NO: 13637); ENST00000619204.4 (SEQ ID NO: 13639); |
| | ENST00000629768.2 (SEQ ID NO: 13641) |
| transcript | ENST00000329875.13 (SEQ ID NO: 13610); ENST00000337943.9 (SEQ ID NO: 13612); |
| | ENST00000402252.6 (SEQ ID NO: 13614); ENST00000403172.8 (SEQ ID NO: 13616); |
| | ENST00000405481.8 (SEQ ID NO: 13618); ENST00000577624.5 (SEQ ID NO: 13620); |
| | ENST00000577756.5 (SEQ ID NO: 13622); ENST00000579366.5 (SEQ ID NO: 13624); |
| | ENST00000579698.5 (SEQ ID NO: 13626); ENST00000581271.5 (SEQ ID NO: 13628); |
| | ENST00000582198.5 (SEQ ID NO: 13630); ENST00000583564.5 (SEQ ID NO: 13632); |
| | ENST00000584848.5 (SEQ ID NO: 13634); ENST00000585215.5 (SEQ ID NO: 13636); |
| | ENST00000585244.1 (SEQ ID NO: 13638); ENST00000619204.4 (SEQ ID NO: 13640); |
| | ENST00000629768.2 (SEQ ID NO: 13642) |

Target: RAD52

| | |
|---|---|
| pre-mRNA | ENST00000228345.9 (SEQ ID NO: 13643); ENST00000358495.8 (SEQ ID NO: 15756); |
| transcript | ENST00000397230.6 (SEQ ID NO: 13646); ENST00000430095.6 (SEQ ID NO: 15757); |
| | ENST00000461568.5 (SEQ ID NO: 15758); ENST00000463750.5 (SEQ ID NO: 15759); |
| | ENST00000468231.5 (SEQ ID NO: 15760); ENST00000481052.5 (SEQ ID NO: 15761); |
| | ENST00000488642.6 (SEQ ID NO: 15762); ENST00000535376.5 (SEQ ID NO: 13654); |
| | ENST00000536177.5 (SEQ ID NO: 15763); ENST00000541619.1 (SEQ ID NO: 13657); |
| | ENST00000542297.1 (SEQ ID NO: 13659); ENST00000542584.5 (SEQ ID NO: 15764); |
| | ENST00000542785.5 (SEQ ID NO: 13662); ENST00000543912.5 (SEQ ID NO: 15765); |
| | ENST00000544742.5 (SEQ ID NO: 13665); ENST00000545564.5 (SEQ ID NO: 13667); |
| | ENST00000545967.1; (SEQ ID NO: 13669) |
| transcript | ENST00000228345.9 (SEQ ID NO: 13644); ENST00000358495.8 (SEQ ID NO: 13645); |
| | ENST00000397230.6 (SEQ ID NO: 13647); ENST00000430095.6 (SEQ ID NO: 13648); |
| | ENST00000461568.5 (SEQ ID NO: 13649); ENST00000463750.5 (SEQ ID NO: 13650); |
| | ENST00000468231.5 (SEQ ID NO: 13651); ENST00000481052.5 (SEQ ID NO: 13652); |
| | ENST00000488642.6 (SEQ ID NO: 13653); ENST00000535376.5 (SEQ ID NO: 13655); |
| | ENST00000536177.5 (SEQ ID NO: 13656); ENST00000541619.1 (SEQ ID NO: 13658); |
| | ENST00000542297.1 (SEQ ID NO: 13660); ENST00000542584.5 (SEQ ID NO: 13661); |
| | ENST00000542785.5 (SEQ ID NO: 13663); ENST00000543912.5 (SEQ ID NO: 13664); |
| | ENST00000544742.5 (SEQ ID NO: 13666); ENST00000545564.5 (SEQ ID NO: 13668); |
| | ENST00000545967.1 (SEQ ID NO: 13670) |

Target: REXO1

| | |
|---|---|
| pre-mRNA | ENST00000170168.9 (SEQ ID NO: 15766); ENST00000586291.1 (SEQ ID NO: 13672); |
| transcript | ENST00000586343.2 (SEQ ID NO: 13674); ENST00000587404.1 (SEQ ID NO: 13676); |
| | ENST00000587524.1 (SEQ ID NO: 13678); ENST00000588743.2 (SEQ ID NO: 13680); |
| | ENST00000590936.5 (SEQ ID NO: 13682); ENST00000643515.1 (SEQ ID NO: 13684) |
| transcript | ENST00000170168.9 (SEQ ID NO: 13671); ENST00000586291.1 (SEQ ID NO: 13673); |
| | ENST00000586343.2 (SEQ ID NO: 13675); ENST00000587404.1 (SEQ ID NO: 13677); |
| | ENST00000587524.1 (SEQ ID NO: 13679); ENST00000588743.2 (SEQ ID NO: 13681); |
| | ENST00000590936.5 (SEQ ID NO: 13683); ENST00000643515.1 (SEQ ID NO: 13685) |

Target: RFX5

| | |
|---|---|
| pre-mRNA | ENST00000290524.8 (SEQ ID NO: 13686); ENST00000368870.6 (SEQ ID NO: 13688); |
| transcript | ENST00000392746.7 (SEQ ID NO: 13690); ENST00000412774.5 (SEQ ID NO: 13692); |
| | ENST00000421986.5 (SEQ ID NO: 13694); ENST00000422595.5 (SEQ ID NO: 13696); |
| | ENST00000430227.5 (SEQ ID NO: 13698); ENST00000435314.5 (SEQ ID NO: 13700); |
| | ENST00000436271.5 (SEQ ID NO: 13702); ENST00000436637.5 (SEQ ID NO: 13704); |
| | ENST00000437327.5 (SEQ ID NO: 13706); ENST00000444392.5 (SEQ ID NO: 13708); |
| | ENST00000450506.5 (SEQ ID NO: 13710); ENST00000452456.1 (SEQ ID NO: 13712); |
| | ENST00000452671.6 (SEQ ID NO: 13714); ENST00000458484.5 (SEQ ID NO: 13716); |
| | ENST00000469513.5 (SEQ ID NO: 13718); ENST00000475144.1 (SEQ ID NO: 13720); |
| | ENST00000478564.5 (SEQ ID NO: 13722); ENST00000479681.5 (SEQ ID NO: 13724); |
| | ENST00000494217.5 (SEQ ID NO: 13726) |
| transcript | ENST00000290524.8 (SEQ ID NO: 13687); ENST00000368870.6 (SEQ ID NO: 13689); |
| | ENST00000392746.7 (SEQ ID NO: 13691); ENST00000412774.5 (SEQ ID NO: 13693); |
| | ENST00000421986.5 (SEQ ID NO: 13695); ENST00000422595.5 (SEQ ID NO: 13697); |
| | ENST00000430227.5 (SEQ ID NO: 13699); ENST00000435314.5 (SEQ ID NO: 13701); |
| | ENST00000436271.5 (SEQ ID NO: 13703); ENST00000436637.5 (SEQ ID NO: 13705); |
| | ENST00000437327.5 (SEQ ID NO: 13707); ENST00000444392.5 (SEQ ID NO: 13709); |
| | ENST00000450506.5 (SEQ ID NO: 13711); ENST00000452456.1 (SEQ ID NO: 13713); |
| | ENST00000452671.6 (SEQ ID NO: 13715); ENST00000458484.5 (SEQ ID NO: 13717); |
| | ENST00000469513.5 (SEQ ID NO: 13719); ENST00000475144.1 (SEQ ID NO: 13721); |
| | ENST00000478564.5 (SEQ ID NO: 13723); ENST00000479681.5 (SEQ ID NO: 13725); |
| | ENST00000494217.5 (SEQ ID NO: 13727) |

Target: RHBDF2

| | |
|---|---|
| pre-mRNA | ENST00000313080.8 (SEQ ID NO: 15767); ENST00000585701.5 (SEQ ID NO: 13729); |
| transcript | ENST00000585989.5 (SEQ ID NO: 13731); ENST00000587640.1 (SEQ ID NO: 13733); |
| | ENST00000589526.5 (SEQ ID NO: 13735); ENST00000589582.5 (SEQ ID NO: 13737); |
| | ENST00000590168.5 (SEQ ID NO: 13739); ENST00000590288.1 (SEQ ID NO: 13741); |
| | ENST00000590322.1 (SEQ ID NO: 13743); ENST00000591192.1 (SEQ ID NO: 13745); |
| | ENST00000591255.5 (SEQ ID NO: 13747); ENST00000591697.5 (SEQ ID NO: 13749); |
| | ENST00000591860.1 (SEQ ID NO: 13751); ENST00000591879.1 (SEQ ID NO: 13753); |
| | ENST00000591885.5 (SEQ ID NO: 15768); ENST00000592123.5 (SEQ ID NO: 13756); |
| | ENST00000592378.5 (SEQ ID NO: 13758); ENST00000593103.1 (SEQ ID NO: 13760) |
| transcript | ENST00000313080.8 (SEQ ID NO: 13728); ENST00000585701.5 (SEQ ID NO: 13730); |
| | ENST00000585989.5 (SEQ ID NO: 13732); ENST00000587640.1 (SEQ ID NO: 13734); |
| | ENST00000589526.5 (SEQ ID NO: 13736); ENST00000589582.5 (SEQ ID NO: 13738); |
| | ENST00000590168.5 (SEQ ID NO: 13740); ENST00000590288.1 (SEQ ID NO: 13742); |
| | ENST00000590322.1 (SEQ ID NO: 13744); ENST00000591192.1 (SEQ ID NO: 13746); |
| | ENST00000591255.5 (SEQ ID NO: 13748); ENST00000591697.5 (SEQ ID NO: 13750); |
| | ENST00000591860.1 (SEQ ID NO: 13752); ENST00000591879.1 (SEQ ID NO: 13754); |
| | ENST00000591885.5 (SEQ ID NO: 13755); ENST00000592123.5 (SEQ ID NO: 13757); |
| | ENST00000592378.5 (SEQ ID NO: 13759); ENST00000593103.1 (SEQ ID NO: 13761) |

Target: RMND1

| | |
|---|---|
| pre-mRNA | ENST00000336451.8 (SEQ ID NO: 15769); ENST00000444024.3 (SEQ ID NO: 15770); |
| transcript | ENST00000491268.2 (SEQ ID NO: 13764); ENST00000622845.5 (SEQ ID NO: 15771); |
| | ENST00000643550.1 (SEQ ID NO: 13767); ENST00000643564.1 (SEQ ID NO: 13769); |
| | ENST00000644054.1 (SEQ ID NO: 15772); ENST00000644711.1 (SEQ ID NO: 15773); |
| | ENST00000645367.1 (SEQ ID NO: 15774); ENST00000645895.1 (SEQ ID NO: 15775); |
| | ENST00000645917.1 (SEQ ID NO: 13775); ENST00000646926.1 (SEQ ID NO: 15776) |
| transcript | ENST00000336451.8 (SEQ ID NO: 13762); ENST00000444024.3 (SEQ ID NO: 13763); |
| | ENST00000491268.2 (SEQ ID NO: 13765); ENST00000622845.5 (SEQ ID NO: 13766); |
| | ENST00000643550.1 (SEQ ID NO: 13768); ENST00000643564.1 (SEQ ID NO: 13770); |
| | ENST00000644054.1 (SEQ ID NO: 13771); ENST00000644711.1 (SEQ ID NO: 13772); |
| | ENST00000645367.1 (SEQ ID NO: 13773); ENST00000645895.1 (SEQ ID NO: 13774); |
| | ENST00000645917.1 (SEQ ID NO: 13776); ENST00000646926.1 (SEQ ID NO: 13777) |

Target: ROBO3

| | |
|---|---|
| pre-mRNA | ENST00000397801.6 (SEQ ID NO: 13778); ENST00000524971.1 (SEQ ID NO: 13780); |
| transcript | ENST00000525304.5 (SEQ ID NO: 13782); ENST00000525448.5 (SEQ ID NO: 13784); |
| | ENST00000525482.5 (SEQ ID NO: 13786); ENST00000526551.5 (SEQ ID NO: 13788); |
| | ENST00000527196.5 (SEQ ID NO: 13790); ENST00000527245.5 (SEQ ID NO: 13792); |
| | ENST00000528068.5 (SEQ ID NO: 13794); ENST00000528144.5 (SEQ ID NO: 13796); |
| | ENST00000528820.5 (SEQ ID NO: 13798); ENST00000529658.5 (SEQ ID NO: 13800); |
| | ENST00000530647.5 (SEQ ID NO: 13802); ENST00000531075.5 (SEQ ID NO: 13804); |
| | ENST00000531119.1 (SEQ ID NO: 13806); ENST00000531545.5 (SEQ ID NO: 13808); |
| | ENST00000531888.1 (SEQ ID NO: 13810); ENST00000532472.1 (SEQ ID NO: 13812); |
| | ENST00000534598.5 (SEQ ID NO: 13814); ENST00000538940.5 (SEQ ID NO: 13816); |
| | ENST00000543966.5; (SEQ ID NO: 13818) |
| transcript | ENST00000397801.6 (SEQ ID NO: 13779); ENST00000524971.1 (SEQ ID NO: 13781); |
| | ENST00000525304.5 (SEQ ID NO: 13783); ENST00000525448.5 (SEQ ID NO: 13785); |
| | ENST00000525482.5 (SEQ ID NO: 13787); ENST00000526551.5 (SEQ ID NO: 13789); |
| | ENST00000527196.5 (SEQ ID NO: 13791); ENST00000527245.5 (SEQ ID NO: 13793); |
| | ENST00000528068.5 (SEQ ID NO: 13795); ENST00000528144.5 (SEQ ID NO: 13797); |
| | ENST00000528820.5 (SEQ ID NO: 13799); ENST00000529658.5 (SEQ ID NO: 13801); |
| | ENST00000530647.5 (SEQ ID NO: 13803); ENST00000531075.5 (SEQ ID NO: 13805); |
| | ENST00000531119.1 (SEQ ID NO: 13807); ENST00000531545.5 (SEQ ID NO: 13809); |
| | ENST00000531888.1 (SEQ ID NO: 13811); ENST00000532472.1 (SEQ ID NO: 13813); |
| | ENST00000534598.5 (SEQ ID NO: 13815); ENST00000538940.5 (SEQ ID NO: 13817); |
| | ENST00000543966.5 (SEQ ID NO: 13819) |

Target: RPGRIP1

| | |
|---|---|
| pre-mRNA | ENST00000382933.8 (SEQ ID NO: 15777); ENST00000400017.6 (SEQ ID NO: 15778); |
| transcript | ENST00000553500.5 (SEQ ID NO: 13822); ENST00000553927.1 (SEQ ID NO: 13824); |
| | ENST00000554303.1 (SEQ ID NO: 13826); ENST00000554750.1 (SEQ ID NO: 13828); |
| | ENST00000555322.5 (SEQ ID NO: 13830); ENST00000555489.5 (SEQ ID NO: 13832); |
| | ENST00000555587.5 (SEQ ID NO: 13834); ENST00000556336.5 (SEQ ID NO: 15779); |
| | ENST00000557351.1 (SEQ ID NO: 13837); ENST00000557606.1 (SEQ ID NO: 13839); |
| | ENST00000557771.5 (SEQ ID NO: 15780) |
| transcript | ENST00000382933.8 (SEQ ID NO: 13820); ENST00000400017.6 (SEQ ID NO: 13821); |
| | ENST00000553500.5 (SEQ ID NO: 13823); ENST00000553927.1 (SEQ ID NO: 13825); |
| | ENST00000554303.1 (SEQ ID NO: 13827); ENST00000554750.1 (SEQ ID NO: 13829); |
| | ENST00000555322.5 (SEQ ID NO: 13831); ENST00000555489.5 (SEQ ID NO: 13833); |
| | ENST00000555587.5 (SEQ ID NO: 13835); ENST00000556336.5 (SEQ ID NO: 13836); |
| | ENST00000557351.1 (SEQ ID NO: 13838); ENST00000557606.1 (SEQ ID NO: 13840); |
| | ENST00000557771.5 (SEQ ID NO: 13841) |

Target: RTTN

| | |
|---|---|
| pre-mRNA | ENST00000255674.11 (SEQ ID NO: 15781); ENST00000578780.2 (SEQ ID NO: 13843); |
| transcript | ENST00000579021.1 (SEQ ID NO: 13845); ENST00000579986.6 (SEQ ID NO: 15782); |
| | ENST00000580034.2 (SEQ ID NO: 13848); ENST00000581161.5 (SEQ ID NO: 15783); |
| | ENST00000581583.1 (SEQ ID NO: 15784); ENST00000581709.1 (SEQ ID NO: 13852); |
| | ENST00000583043.5 (SEQ ID NO: 15785); ENST00000583765.1 (SEQ ID NO: 13855); |
| | ENST00000638251.1 (SEQ ID NO: 15786); ENST00000638298.1 (SEQ ID NO: 13858); |
| | ENST00000638799.1 (SEQ ID NO: 13860); ENST00000639128.1 (SEQ ID NO: 15787); |
| | ENST00000639487.1 (SEQ ID NO: 15788); ENST00000640376.1 (SEQ ID NO: 15789); |
| | ENST00000640393.1 (SEQ ID NO: 13865); ENST00000640408.1 (SEQ ID NO: 15790); |
| | ENST00000640525.1 (SEQ ID NO: 13868); ENST00000640654.1 (SEQ ID NO: 13870); |
| | ENST00000640736.1 (SEQ ID NO: 13872); ENST00000640769.2 (SEQ ID NO: 15791); |
| | ENST00000640931.1; (SEQ ID NO: 15792) |
| transcript | ENST00000255674.11 (SEQ ID NO: 13842); ENST00000578780.2 (SEQ ID NO: 13844); |
| | ENST00000579021.1 (SEQ ID NO: 13846); ENST00000579986.6 (SEQ ID NO: 13847); |
| | ENST00000580034.2 (SEQ ID NO: 13849); ENST00000581161.5 (SEQ ID NO: 13850); |
| | ENST00000581583.1 (SEQ ID NO: 13851); ENST00000581709.1 (SEQ ID NO: 13853); |
| | ENST00000583043.5 (SEQ ID NO: 13854); ENST00000583765.1 (SEQ ID NO: 13856); |
| | ENST00000638251.1 (SEQ ID NO: 13857); ENST00000638298.1 (SEQ ID NO: 13859); |
| | ENST00000638799.1 (SEQ ID NO: 13861); ENST00000639128.1 (SEQ ID NO: 13862); |
| | ENST00000639487.1 (SEQ ID NO: 13863); ENST00000640376.1 (SEQ ID NO: 13864); |
| | ENST00000640393.1 (SEQ ID NO: 13866); ENST00000640408.1 (SEQ ID NO: 13867); |
| | ENST00000640525.1 (SEQ ID NO: 13869); ENST00000640654.1 (SEQ ID NO: 13871); |
| | ENST00000640736.1 (SEQ ID NO: 13873); ENST00000640769.2 (SEQ ID NO: 13874); |
| | ENST00000640931.1 (SEQ ID NO: 13875) |

Target: RUFY3

| | |
|---|---|
| pre-mRNA | ENST00000226328.8 (SEQ ID NO: 15793); ENST00000381006.8 (SEQ ID NO: 15794); |
| transcript | ENST00000417478.6 (SEQ ID NO: 15795); ENST00000502653.5 (SEQ ID NO: 15796); |
| | ENST00000503025.5 (SEQ ID NO: 13880); ENST00000503876.5 (SEQ ID NO: 15797); |
| | ENST00000504805.6 (SEQ ID NO: 13883); ENST00000507333.5 (SEQ ID NO: 13885); |
| | ENST00000512103.5 (SEQ ID NO: 13887); ENST00000512331.5 (SEQ ID NO: 13889); |
| | ENST00000513593.1 (SEQ ID NO: 13891); ENST00000513597.5 (SEQ ID NO: 15798); |
| | ENST00000514898.1 (SEQ ID NO: 13894); ENST00000515442.1 (SEQ ID NO: 13896); |
| | ENST00000515479.5 (SEQ ID NO: 13898) |
| transcript | ENST00000226328.8 (SEQ ID NO: 13876); ENST00000381006.8 (SEQ ID NO: 13877); |
| | ENST00000417478.6 (SEQ ID NO: 13878); ENST00000502653.5 (SEQ ID NO: 13879); |
| | ENST00000503025.5 (SEQ ID NO: 13881); ENST00000503876.5 (SEQ ID NO: 13882); |
| | ENST00000504805.6 (SEQ ID NO: 13884); ENST00000507333.5 (SEQ ID NO: 13886); |
| | ENST00000512103.5 (SEQ ID NO: 13888); ENST00000512331.5 (SEQ ID NO: 13890); |
| | ENST00000513593.1 (SEQ ID NO: 13892); ENST00000513597.5 (SEQ ID NO: 13893); |
| | ENST00000514898.1 (SEQ ID NO: 13895); ENST00000515442.1 (SEQ ID NO: 13897); |
| | ENST00000515479.5 (SEQ ID NO: 13899) |

Target: SEMA3B

| | |
|---|---|
| pre-mRNA | ENST00000416295.1 (SEQ ID NO: 13900); ENST00000418576.3 (SEQ ID NO: 13902); |
| transcript | ENST00000419007.5 (SEQ ID NO: 13904); ENST00000433753.4 (SEQ ID NO: 13906); |
| | ENST00000434030.1 (SEQ ID NO: 13908); ENST00000439487.5 (SEQ ID NO: 13910); |
| | ENST00000441915.5 (SEQ ID NO: 13912); ENST00000456210.5 (SEQ ID NO: 13914); |
| | ENST00000456560.6 (SEQ ID NO: 13916); ENST00000611067.4 (SEQ ID NO: 13918); |
| | ENST00000612509.4 (SEQ ID NO: 13920); ENST00000616701.5 (SEQ ID NO: 13922); |
| | ENST00000618865.4 (SEQ ID NO: 13924); ENST00000619119.4 (SEQ ID NO: 13926); |
| | ENST00000621029.4 (SEQ ID NO: 13928) |
| transcript | ENST00000416295.1 (SEQ ID NO: 13901); ENST00000418576.3 (SEQ ID NO: 13903); |
| | ENST00000419007.5 (SEQ ID NO: 13905); ENST00000433753.4 (SEQ ID NO: 13907); |
| | ENST00000434030.1 (SEQ ID NO: 13909); ENST00000439487.5 (SEQ ID NO: 13911); |
| | ENST00000441915.5 (SEQ ID NO: 13913); ENST00000456210.5 (SEQ ID NO: 13915); |
| | ENST00000456560.6 (SEQ ID NO: 13917); ENST00000611067.4 (SEQ ID NO: 13919); |
| | ENST00000612509.4 (SEQ ID NO: 13921); ENST00000616701.5 (SEQ ID NO: 13923); |
| | ENST00000618865.4 (SEQ ID NO: 13925); ENST00000619119.4 (SEQ ID NO: 13927); |
| | ENST00000621029.4 (SEQ ID NO: 13929) |

Target: SEMA3F

| | |
|---|---|
| pre-mRNA | ENST00000002829.8 (SEQ ID NO: 15799); ENST00000413852.5 (SEQ ID NO: 15800); |
| transcript | ENST00000414301.5 (SEQ ID NO: 13932); ENST00000420831.1 (SEQ ID NO: 13934); |
| | ENST00000426511.5 (SEQ ID NO: 13936); ENST00000434342.5 (SEQ ID NO: 15801); |
| | ENST00000450338.5 (SEQ ID NO: 13939); ENST00000470737.1 (SEQ ID NO: 13941); |
| | ENST00000493743.1 (SEQ ID NO: 13943) |
| transcript | ENST00000002829.8 (SEQ ID NO: 13930); ENST00000413852.5 (SEQ ID NO: 13931); |
| | ENST00000414301.5 (SEQ ID NO: 13933); ENST00000420831.1 (SEQ ID NO: 13935); |
| | ENST00000426511.5 (SEQ ID NO: 13937); ENST00000434342.5 (SEQ ID NO: 13938); |
| | ENST00000450338.5 (SEQ ID NO: 13940); ENST00000470737.1 (SEQ ID NO: 13942); |
| | ENST00000493743.1 (SEQ ID NO: 13944) |

Target: SH2D3A

| | |
|---|---|
| pre-mRNA | ENST00000245908.11 (SEQ ID NO: 13945); ENST00000437152.7 (SEQ ID NO: 13947); |
| transcript | ENST00000595369.1 (SEQ ID NO: 13949); ENST00000595681.5 (SEQ ID NO: 13951); |
| | ENST00000597168.1 (SEQ ID NO: 13953); ENST00000597254.1 (SEQ ID NO: 13955); |
| | ENST00000597687.1 (SEQ ID NO: 13957); ENST00000599563.5 (SEQ ID NO: 13959) |
| transcript | ENST00000245908.11 (SEQ ID NO: 13946); ENST00000437152.7 (SEQ ID NO: 13948); |
| | ENST00000595369.1 (SEQ ID NO: 13950); ENST00000595681.5 (SEQ ID NO: 13952); |
| | ENST00000597168.1 (SEQ ID NO: 13954); ENST00000597254.1 (SEQ ID NO: 13956); |
| | ENST00000597687.1 (SEQ ID NO: 13958); ENST00000599563.5 (SEQ ID NO: 13960) |

Target: SIK3

| | |
|---|---|
| pre-mRNA | ENST00000375300.6 (SEQ ID NO: 15802); ENST00000413553.1 (SEQ ID NO: 15803); |
| transcript | ENST00000415541.5 (SEQ ID NO: 15804); ENST00000445177.5 (SEQ ID NO: 15805); |
| | ENST00000446921.6 (SEQ ID NO: 15806); ENST00000454905.5 (SEQ ID NO: 13966); |
| | ENST00000465421.5 (SEQ ID NO: 13968); ENST00000472648.1 (SEQ ID NO: 13970); |
| | ENST00000480222.1 (SEQ ID NO: 13972); ENST00000480468.1 (SEQ ID NO: 13974); |
| | ENST00000485363.1 (SEQ ID NO: 15807); ENST00000488337.5 (SEQ ID NO: 13977); |
| | ENST00000497049.5; (SEQ ID NO: 15808) |
| transcript | ENST00000375300.6 (SEQ ID NO: 13961); ENST00000413553.1 (SEQ ID NO: 13962); |
| | ENST00000415541.5 (SEQ ID NO: 13963); ENST00000445177.5 (SEQ ID NO: 13964); |
| | ENST00000446921.6 (SEQ ID NO: 13965); ENST00000454905.5 (SEQ ID NO: 13967); |
| | ENST00000465421.5 (SEQ ID NO: 13969); ENST00000472648.1 (SEQ ID NO: 13971); |
| | ENST00000480222.1 (SEQ ID NO: 13973); ENST00000480468.1 (SEQ ID NO: 13975); |
| | ENST00000485363.1 (SEQ ID NO: 13976); ENST00000488337.5 (SEQ ID NO: 13978); |
| | ENST00000497049.5 (SEQ ID NO: 13979) |

Target: SIRT3

| | |
|---|---|
| pre-mRNA | ENST00000382743.9 (SEQ ID NO: 13980); ENST00000524564.5 (SEQ ID NO: 13982); |
| transcript | ENST00000525237.1 (SEQ ID NO: 13984); ENST00000525319.5 (SEQ ID NO: 13986); |
| | ENST00000525776.1 (SEQ ID NO: 13988); ENST00000526854.5 (SEQ ID NO: 13990); |
| | ENST00000528469.1 (SEQ ID NO: 13992); ENST00000528702.5 (SEQ ID NO: 13994); |
| | ENST00000529055.5 (SEQ ID NO: 13996); ENST00000529382.5 (SEQ ID NO: 13998); |
| | ENST00000529937.1 (SEQ ID NO: 14000); ENST00000530067.1 (SEQ ID NO: 14002); |
| | ENST00000531753.5 (SEQ ID NO: 14004); ENST00000532837.5 (SEQ ID NO: 14006); |
| | ENST00000532956.5 (SEQ ID NO: 14008); ENST00000534381.1 (SEQ ID NO: 14010) |
| transcript | ENST00000382743.9 (SEQ ID NO: 13981); ENST00000524564.5 (SEQ ID NO: 13983); |
| | ENST00000525237.1 (SEQ ID NO: 13985); ENST00000525319.5 (SEQ ID NO: 13987); |
| | ENST00000525776.1 (SEQ ID NO: 13989); ENST00000526854.5 (SEQ ID NO: 13991); |
| | ENST00000528469.1 (SEQ ID NO: 13993); ENST00000528702.5 (SEQ ID NO: 13995); |
| | ENST00000529055.5 (SEQ ID NO: 13997); ENST00000529382.5 (SEQ ID NO: 13999); |
| | ENST00000529937.1 (SEQ ID NO: 14001); ENST00000530067.1 (SEQ ID NO: 14003); |
| | ENST00000531753.5 (SEQ ID NO: 14005); ENST00000532837.5 (SEQ ID NO: 14007); |
| | ENST00000532956.5 (SEQ ID NO: 14009); ENST00000534381.1 (SEQ ID NO: 14011) |

Target: SKTV2L

| | |
|---|---|
| pre-mRNA | ENST00000375394.7 (SEQ ID NO: 14012); ENST00000461073.5 (SEQ ID NO: 14014); |
| transcript | ENST00000461915.5 (SEQ ID NO: 14016); ENST00000465703.5 (SEQ ID NO: 14018); |
| | ENST00000466290.1 (SEQ ID NO: 14020); ENST00000470453.1 (SEQ ID NO: 14022); |
| | ENST00000471818.1 (SEQ ID NO: 14024); ENST00000474839.5 (SEQ ID NO: 14026); |
| | ENST00000483553.5 (SEQ ID NO: 14028); ENST00000484835.1 (SEQ ID NO: 14030); |
| | ENST00000485349.5 (SEQ ID NO: 14032); ENST00000488648.5 (SEQ ID NO: 14034); |
| | ENST00000491994.1 (SEQ ID NO: 14036); ENST00000492900.1 (SEQ ID NO: 14038); |
| | ENST00000494058.5 (SEQ ID NO: 14040); ENST00000628157.1 (SEQ ID NO: 14042) |
| transcript | ENST00000375394.7 (SEQ ID NO: 14013); ENST00000461073.5 (SEQ ID NO: 14015); |
| | ENST00000461915.5 (SEQ ID NO: 14017); ENST00000465703.5 (SEQ ID NO: 14019); |
| | ENST00000466290.1 (SEQ ID NO: 14021); ENST00000470453.1 (SEQ ID NO: 14023); |
| | ENST00000471818.1 (SEQ ID NO: 14025); ENST00000474839.5 (SEQ ID NO: 14027); |
| | ENST00000483553.5 (SEQ ID NO: 14029); ENST00000484835.1 (SEQ ID NO: 14031); |
| | ENST00000485349.5 (SEQ ID NO: 14033); ENST00000488648.5 (SEQ ID NO: 14035); |
| | ENST00000491994.1 (SEQ ID NO: 14037); ENST00000492900.1 (SEQ ID NO: 14039); |
| | ENST00000494058.5 (SEQ ID NO: 14041); ENST00000628157.1 (SEQ ID NO: 14043) |

Target: SLC2A13

| | |
|---|---|
| pre-mRNA | ENST00000280871.9 (SEQ ID NO: 14044); ENST00000380858.1 (SEQ ID NO: 14046); |
| transcript | ENST00000465517.1 (SEQ ID NO: 14048); ENST00000505338.1 (SEQ ID NO: 14050) |
| transcript | ENST00000280871.9 (SEQ ID NO: 14045); ENST00000380858.1 (SEQ ID NO: 14047); |
| | ENST00000465517.1 (SEQ ID NO: 14049); ENST00000505338.1 (SEQ ID NO: 14051) |

Target: SLC12A7

| | |
|---|---|
| pre-mRNA | ENST00000264930.10 (SEQ ID NO: 14052); ENST00000504576.2 (SEQ ID NO: 14054); |
| transcript | ENST00000510943.3 (SEQ ID NO: 14056); ENST00000513223.2 (SEQ ID NO: 14058); |
| | ENST00000514994.1 (SEQ ID NO: 14060); ENST00000634447.1 (SEQ ID NO: 14062) |
| transcript | ENST00000264930.10 (SEQ ID NO: 14053); ENST00000504576.2 (SEQ ID NO: 14055); |
| | ENST00000510943.3 (SEQ ID NO: 14057); ENST00000513223.2 (SEQ ID NO: 14059); |
| | ENST00000514994.1 (SEQ ID NO: 14061); ENST00000634447.1 (SEQ ID NO: 14063) |

Target: SLC22A3

| | |
|---|---|
| pre-mRNA | ENST00000275300.3 (SEQ ID NO: 14064) |
| transcript | |
| transcript | ENST00000275300.3 (SEQ ID NO: 14065) |

Target: SLC25A13

| | |
|---|---|
| pre-mRNA | ENST00000265631.9 (SEQ ID NO: 15809); ENST00000416240.6 (SEQ ID NO: 15810); |
| transcript | ENST00000472162.2 (SEQ ID NO: 15811); ENST00000484495.5 (SEQ ID NO: 14069); |
| | ENST00000487710.1 (SEQ ID NO: 14071); ENST00000490072.5 (SEQ ID NO: 15812); |
| | ENST00000492869.1 (SEQ ID NO: 14074); ENST00000494085.1 (SEQ ID NO: 14076) |
| transcript | ENST00000265631.9 (SEQ ID NO: 14066); ENST00000416240.6 (SEQ ID NO: 14067); |
| | ENST00000472162.2 (SEQ ID NO: 14068); ENST00000484495.5 (SEQ ID NO: 14070); |
| | ENST00000487710.1 (SEQ ID NO: 14072); ENST00000490072.5 (SEQ ID NO: 14073); |
| | ENST00000492869.1 (SEQ ID NO: 14075); ENST00000494085.1 (SEQ ID NO: 14077) |

Target: SLC25A37

| | |
|---|---|
| pre-mRNA | ENST00000290075.10 (SEQ ID NO: 15813); ENST00000417331.6 (SEQ ID NO: 15814); |
| transcript | ENST00000517923.1 (SEQ ID NO: 14080); ENST00000518881.5 (SEQ ID NO: 15815); |
| | ENST00000519192.1 (SEQ ID NO: 14083); ENST00000519973.6 (SEQ ID NO: 15816); |
| | ENST00000520654.1 (SEQ ID NO: 15817); ENST00000520949.1 (SEQ ID NO: 14087); |
| | ENST00000521637.1 (SEQ ID NO: 14089); ENST00000522164.5 (SEQ ID NO: 14091); |
| | ENST00000523883.5 (SEQ ID NO: 15818); ENST00000523930.1 (SEQ ID NO: 15819) |
| transcript | ENST00000290075.10 (SEQ ID NO: 14078); ENST00000417331.6 (SEQ ID NO: 14079); |
| | ENST00000517923.1 (SEQ ID NO: 14081); ENST00000518881.5 (SEQ ID NO: 14082); |
| | ENST00000519192.1 (SEQ ID NO: 14084); ENST00000519973.6 (SEQ ID NO: 14085); |
| | ENST00000520654.1 (SEQ ID NO: 14086); ENST00000520949.1 (SEQ ID NO: 14088); |
| | ENST00000521637.1 (SEQ ID NO: 14090); ENST00000522164.5 (SEQ ID NO: 14092); |
| | ENST00000523883.5 (SEQ ID NO: 14093); ENST00000523930.1 (SEQ ID NO: 14094) |

Target: SLC27A5

| | |
|---|---|
| pre-mRNA | ENST00000263093.7 (SEQ ID NO: 14095); ENST00000593745.1 (SEQ ID NO: 14097); |
| transcript | ENST00000594683.1 (SEQ ID NO: 14099); ENST00000594786.1 (SEQ ID NO: 14101); |
| | ENST00000595851.5 (SEQ ID NO: 14103); ENST00000599700.1 (SEQ ID NO: 14105); |
| | ENST00000601355.1 (SEQ ID NO: 14107); ENST00000601997.1; (SEQ ID NO: 14109) |
| transcript | ENST00000263093.7 (SEQ ID NO: 14096); ENST00000593745.1 (SEQ ID NO: 14098); |
| | ENST00000594683.1 (SEQ ID NO: 14100); ENST00000594786.1 (SEQ ID NO: 14102); |
| | ENST00000595851.5 (SEQ ID NO: 14104); ENST00000599700.1 (SEQ ID NO: 14106); |
| | ENST00000601355.1 (SEQ ID NO: 14108); ENST00000601997.1 (SEQ ID NO: 14110) |

Target: SLC30A9

| | |
|---|---|
| pre-mRNA | ENST00000264451.12 (SEQ ID NO: 14111); ENST00000505523.1 (SEQ ID NO: 14113); |
| transcript | ENST00000509683.5 (SEQ ID NO: 14115); ENST00000510460.1 (SEQ ID NO: 14117); |
| | ENST00000513699.5 (SEQ ID NO: 14119) |
| transcript | ENST00000264451.12 (SEQ ID NO: 14112); ENST00000505523.1 (SEQ ID NO: 14114); |
| | ENST00000509683.5 (SEQ ID NO: 14116); ENST00000510460.1 (SEQ ID NO: 14118); |
| | ENST00000513699.5 (SEQ ID NO: 14120) |

Target: SLC30A10

| | |
|---|---|
| pre-mRNA | ENST00000356609.2 (SEQ ID NO: 14121); ENST00000366926.3 (SEQ ID NO: 14123); |
| transcript | ENST00000484079.1 (SEQ ID NO: 14125); ENST00000484239.5 (SEQ ID NO: 14127) |
| transcript | ENST00000356609.2 (SEQ ID NO: 14122); ENST00000366926.3 (SEQ ID NO: 14124); |
| | ENST00000484079.1 (SEQ ID NO: 14126); ENST00000484239.5 (SEQ ID NO: 14128) |

Target: SMPD1

| | |
|---|---|
| pre-mRNA | ENST00000342245.9 (SEQ ID NO: 14129); ENST00000526280.1 (SEQ ID NO: 14131); |
| transcript | ENST00000527275.5 (SEQ ID NO: 14133); ENST00000530395.1 (SEQ ID NO: 14135); |
| | ENST00000531303.5 (SEQ ID NO: 14137); ENST00000531336.1 (SEQ ID NO: 14139); |
| | ENST00000532367.1 (SEQ ID NO: 14141); ENST00000533123.5 (SEQ ID NO: 14143); |
| | ENST00000533196.1 (SEQ ID NO: 14145); ENST00000534405.5 (SEQ ID NO: 14147) |
| transcript | ENST00000342245.9 (SEQ ID NO: 14130); ENST00000526280.1 (SEQ ID NO: 14132); |
| | ENST00000527275.5 (SEQ ID NO: 14134); ENST00000530395.1 (SEQ ID NO: 14136); |
| | ENST00000531303.5 (SEQ ID NO: 14138); ENST00000531336.1 (SEQ ID NO: 14140); |
| | ENST00000532367.1 (SEQ ID NO: 14142); ENST00000533123.5 (SEQ ID NO: 14144); |
| | ENST00000533196.1 (SEQ ID NO: 14146); ENST00000534405.5 (SEQ ID NO: 14148) |

Target: SMTN

| | |
|---|---|
| pre-mRNA | ENST00000333137.11 (SEQ ID NO: 14149); ENST00000347557.6 (SEQ ID NO: 14151); |
| transcript | ENST00000358743.5 (SEQ ID NO: 14153); ENST00000404574.5 (SEQ ID NO: 14155); |
| | ENST00000416786.5 (SEQ ID NO: 14157); ENST00000422839.5 (SEQ ID NO: 14159); |
| | ENST00000426927.5 (SEQ ID NO: 14161); ENST00000431481.1 (SEQ ID NO: 14163); |
| | ENST00000432777.5 (SEQ ID NO: 14165); ENST00000438223.5 (SEQ ID NO: 14167); |
| | ENST00000440425.5 (SEQ ID NO: 14169); ENST00000455608.5 (SEQ ID NO: 14171); |
| | ENST00000460658.5 (SEQ ID NO: 14173); ENST00000466272.1 (SEQ ID NO: 14175); |
| | ENST00000472911.1 (SEQ ID NO: 14177); ENST00000475548.5 (SEQ ID NO: 14179); |
| | ENST00000482444.5 (SEQ ID NO: 14181); ENST00000489337.5 (SEQ ID NO: 14183); |
| | ENST00000493335.5 (SEQ ID NO: 14185); ENST00000497697.5 (SEQ ID NO: 14187); |
| | ENST00000504335.1 (SEQ ID NO: 14189); ENST00000612341.4 (SEQ ID NO: 14191); |
| | ENST00000619644.4 (SEQ ID NO: 14193); ENST00000624247.1 (SEQ ID NO: 14195) |
| transcript | ENST00000333137.11 (SEQ ID NO: 14150); ENST00000347557.6 (SEQ ID NO: 14152); |
| | ENST00000358743.5 (SEQ ID NO: 14154); ENST00000404574.5 (SEQ ID NO: 14156); |
| | ENST00000416786.5 (SEQ ID NO: 14158); ENST00000422839.5 (SEQ ID NO: 14160); |
| | ENST00000426927.5 (SEQ ID NO: 14162); ENST00000431481.1 (SEQ ID NO: 14164); |
| | ENST00000432777.5 (SEQ ID NO: 14166); ENST00000438223.5 (SEQ ID NO: 14168); |
| | ENST00000440425.5 (SEQ ID NO: 14170); ENST00000455608.5 (SEQ ID NO: 14172); |
| | ENST00000460658.5 (SEQ ID NO: 14174); ENST00000466272.1 (SEQ ID NO: 14176); |
| | ENST00000472911.1 (SEQ ID NO: 14178); ENST00000475548.5 (SEQ ID NO: 14180); |
| | ENST00000482444.5 (SEQ ID NO: 14182); ENST00000489337.5 (SEQ ID NO: 14184); |
| | ENST00000493335.5 (SEQ ID NO: 14186); ENST00000497697.5 (SEQ ID NO: 14188); |
| | ENST00000504335.1 (SEQ ID NO: 14190); ENST00000612341.4 (SEQ ID NO: 14192); |
| | ENST00000619644.4 (SEQ ID NO: 14194); ENST00000624247.1 (SEQ ID NO: 14196) |

Target: SNRPB

| | |
|---|---|
| pre-mRNA | ENST00000381342.6 (SEQ ID NO: 14197); ENST00000438552.6 (SEQ ID NO: 14199); |
| transcript | ENST00000474384.2 (SEQ ID NO: 14201) |
| transcript | ENST00000381342.6 (SEQ ID NO: 14198); ENST00000438552.6 (SEQ ID NO: 14200); |
| | ENST00000474384.2 (SEQ ID NO: 14202) |

Target: SP140

| | |
|---|---|
| pre-mRNA | ENST00000343805.10 (SEQ ID NO: 15820); ENST00000373645.3 (SEQ ID NO: 14204); |
| transcript | ENST00000392045.8 (SEQ ID NO: 15821); ENST00000417495.7 (SEQ ID NO: 15822); |
| | ENST00000420434.7 (SEQ ID NO: 15823); ENST00000441657.1 (SEQ ID NO: 14209); |
| | ENST00000456542.5 (SEQ ID NO: 15824); ENST00000473711.1 (SEQ ID NO: 14212); |
| | ENST00000476126.1 (SEQ ID NO: 14214); ENST00000479539.1 (SEQ ID NO: 14216); |
| | ENST00000486750.1 (SEQ ID NO: 14218); ENST00000538494.1 (SEQ ID NO: 14220); |
| | ENST00000543928.5 (SEQ ID NO: 14222); ENST00000544128.5 (SEQ ID NO: 14224) |
| transcript | ENST00000343805.10 (SEQ ID NO: 14203); ENST00000373645.3 (SEQ ID NO: 14205); |
| | ENST00000392045.8 (SEQ ID NO: 14206); ENST00000417495.7 (SEQ ID NO: 14207); |
| | ENST00000420434.7 (SEQ ID NO: 14208); ENST00000441657.1 (SEQ ID NO: 14210); |
| | ENST00000456542.5 (SEQ ID NO: 14211); ENST00000473711.1 (SEQ ID NO: 14213); |
| | ENST00000476126.1 (SEQ ID NO: 14215); ENST00000479539.1 (SEQ ID NO: 14217); |
| | ENST00000486750.1 (SEQ ID NO: 14219); ENST00000538494.1 (SEQ ID NO: 14221); |
| | ENST00000543928.5 (SEQ ID NO: 14223); ENST00000544128.5 (SEQ ID NO: 14225) |

Target: STAMBP

| | |
|---|---|
| pre-mRNA | ENST00000339566.7 (SEQ ID NO: 15825); ENST00000394070.7 (SEQ ID NO: 15826); |
| transcript | ENST00000394073.5 (SEQ ID NO: 15827); ENST00000409707.5 (SEQ ID NO: 15828); |
| | ENST00000424659.5 (SEQ ID NO: 14230); ENST00000432295.6 (SEQ ID NO: 14232); |
| | ENST00000452725.5 (SEQ ID NO: 14234); ENST00000478946.1 (SEQ ID NO: 14236); |
| | ENST00000486458.1 (SEQ ID NO: 14238); ENST00000487811.1 (SEQ ID NO: 14240); |
| | ENST00000536064.1 (SEQ ID NO: 14242) |
| transcript | ENST00000339566.7 (SEQ ID NO: 14226); ENST00000394070.7 (SEQ ID NO: 14227); |
| | ENST00000394073.5 (SEQ ID NO: 14228); ENST00000409707.5 (SEQ ID NO: 14229); |
| | ENST00000424659.5 (SEQ ID NO: 14231); ENST00000432295.6 (SEQ ID NO: 14233); |
| | ENST00000452725.5 (SEQ ID NO: 14235); ENST00000478946.1 (SEQ ID NO: 14237); |
| | ENST00000486458.1 (SEQ ID NO: 14239); ENST00000487811.1 (SEQ ID NO: 14241); |
| | ENST00000536064.1 (SEQ ID NO: 14243) |

Target: STXBP2

| | |
|---|---|
| pre-mRNA | ENST00000221283.10 (SEQ ID NO: 14244); ENST00000414284.6 (SEQ ID NO: 14246); |
| transcript | ENST00000441779.6 (SEQ ID NO: 14248); ENST00000593535.5 (SEQ ID NO: 14250); |
| | ENST00000593854.5 (SEQ ID NO: 14252); ENST00000594221.5 (SEQ ID NO: 14254); |
| | ENST00000595181.5 (SEQ ID NO: 14256); ENST00000595800.1 (SEQ ID NO: 14258); |
| | ENST00000595861.1 (SEQ ID NO: 14260); ENST00000595950.5 (SEQ ID NO: 14262); |
| | ENST00000597068.5 (SEQ ID NO: 14264); ENST00000597467.1 (SEQ ID NO: 14266); |
| | ENST00000599278.1 (SEQ ID NO: 14268); ENST00000599400.1 (SEQ ID NO: 14270); |
| | ENST00000599558.1 (SEQ ID NO: 14272); ENST00000599648.1 (SEQ ID NO: 14274); |
| | ENST00000599737.5 (SEQ ID NO: 14276); ENST00000599905.1 (SEQ ID NO: 14278); |
| | ENST00000600702.5 (SEQ ID NO: 14280); ENST00000601061.1 (SEQ ID NO: 14282); |
| | ENST00000602355.1 (SEQ ID NO: 14284); ENST00000612033.1 (SEQ ID NO: 14286) |
| transcript | ENST00000221283.10 (SEQ ID NO: 14245); ENST00000414284.6 (SEQ ID NO: 14247); |
| | ENST00000441779.6 (SEQ ID NO: 14249); ENST00000593535.5 (SEQ ID NO: 14251); |
| | ENST00000593854.5 (SEQ ID NO: 14253); ENST00000594221.5 (SEQ ID NO: 14255); |
| | ENST00000595181.5 (SEQ ID NO: 14257); ENST00000595800.1 (SEQ ID NO: 14259); |
| | ENST00000595861.1 (SEQ ID NO: 14261); ENST00000595950.5 (SEQ ID NO: 14263); |
| | ENST00000597068.5 (SEQ ID NO: 14265); ENST00000597467.1 (SEQ ID NO: 14267); |
| | ENST00000599278.1 (SEQ ID NO: 14269); ENST00000599400.1 (SEQ ID NO: 14271); |
| | ENST00000599558.1 (SEQ ID NO: 14273); ENST00000599648.1 (SEQ ID NO: 14275); |
| | ENST00000599737.5 (SEQ ID NO: 14277); ENST00000599905.1 (SEQ ID NO: 14279); |
| | ENST00000600702.5 (SEQ ID NO: 14281); ENST00000601061.1 (SEQ ID NO: 14283); |
| | ENST00000602355.1 (SEQ ID NO: 14285); ENST00000612033.1 (SEQ ID NO: 14287) |

Target: SULF2

| | |
|---|---|
| pre-mRNA | ENST00000359930.8 (SEQ ID NO: 15829); ENST00000433632.1 (SEQ ID NO: 14289); |
| transcript | ENST00000437955.1 (SEQ ID NO: 15830); ENST00000463221.2 (SEQ ID NO: 15831); |
| | ENST00000465769.1 (SEQ ID NO: 14293); ENST00000467815.5 (SEQ ID NO: 15832); |
| | ENST00000474450.5 (SEQ ID NO: 15833); ENST00000478766.1 (SEQ ID NO: 15834); |
| | ENST00000479472.1 (SEQ ID NO: 14298); ENST00000479970.1 (SEQ ID NO: 14300); |
| | ENST00000484875.5 (SEQ ID NO: 15835); ENST00000495544.5 (SEQ ID NO: 14303) |
| transcript | ENST00000359930.8 (SEQ ID NO: 14288); ENST00000433632.1 (SEQ ID NO: 14290); |
| | ENST00000437955.1 (SEQ ID NO: 14291); ENST00000463221.2 (SEQ ID NO: 14292); |
| | ENST00000465769.1 (SEQ ID NO: 14294); ENST00000467815.5 (SEQ ID NO: 14295); |
| | ENST00000474450.5 (SEQ ID NO: 14296); ENST00000478766.1 (SEQ ID NO: 14297); |
| | ENST00000479472.1 (SEQ ID NO: 14299); ENST00000479970.1 (SEQ ID NO: 14301); |
| | ENST00000484875.5 (SEQ ID NO: 14302); ENST00000495544.5 (SEQ ID NO: 14304) |

Target: SYNGAP1

| | |
|---|---|
| pre-mRNA | ENST00000293748.9 (SEQ ID NO: 15836); ENST00000418600.7 (SEQ ID NO: 15837); |
| transcript | ENST00000428982.4 (SEQ ID NO: 14307); ENST00000449372.7 (SEQ ID NO: 15838); |
| | ENST00000470232.1 (SEQ ID NO: 14310); ENST00000479510.2 (SEQ ID NO: 14312); |
| | ENST00000628646.2 (SEQ ID NO: 14314); ENST00000629380.3 (SEQ ID NO: 15839); |
| | ENST00000635885.1 (SEQ ID NO: 14317); ENST00000636075.1 (SEQ ID NO: 14319); |
| | ENST00000636116.1 (SEQ ID NO: 14321); ENST00000636146.1 (SEQ ID NO: 14323); |
| | ENST00000636193.1 (SEQ ID NO: 14325); ENST00000636436.1 (SEQ ID NO: 14327); |
| | ENST00000636443.1 (SEQ ID NO: 14329); ENST00000636640.1 (SEQ ID NO: 14331); |
| | ENST00000636731.1 (SEQ ID NO: 14333); ENST00000636905.1 (SEQ ID NO: 14335); |
| | ENST00000637052.1 (SEQ ID NO: 14337); ENST00000637194.1 (SEQ ID NO: 14339); |
| | ENST00000637490.1 (SEQ ID NO: 14341); ENST00000637587.1 (SEQ ID NO: 14343); |
| | ENST00000637671.1 (SEQ ID NO: 14345); ENST00000637721.1 (SEQ ID NO: 14347); |
| | ENST00000637911.1 (SEQ ID NO: 14349); ENST00000638127.1 (SEQ ID NO: 14351); |
| | ENST00000638142.2 (SEQ ID NO: 14353); ENST00000644458.1 (SEQ ID NO: 15840); |
| | ENST00000645250.1 (SEQ ID NO: 14356); ENST00000646630.1 (SEQ ID NO: 15841) |
| transcript | ENST00000293748.9 (SEQ ID NO: 14305); ENST00000418600.7 (SEQ ID NO: 14306); |
| | ENST00000428982.4 (SEQ ID NO: 14308); ENST00000449372.7 (SEQ ID NO: 14309); |
| | ENST00000470232.1 (SEQ ID NO: 14311); ENST00000479510.2 (SEQ ID NO: 14313); |
| | ENST00000628646.2 (SEQ ID NO: 14315); ENST00000629380.3 (SEQ ID NO: 14316); |
| | ENST00000635885.1 (SEQ ID NO: 14318); ENST00000636075.1 (SEQ ID NO: 14320); |
| | ENST00000636116.1 (SEQ ID NO: 14322); ENST00000636146.1 (SEQ ID NO: 14324); |
| | ENST00000636193.1 (SEQ ID NO: 14326); ENST00000636436.1 (SEQ ID NO: 14328); |
| | ENST00000636443.1 (SEQ ID NO: 14330); ENST00000636640.1 (SEQ ID NO: 14332); |
| | ENST00000636731.1 (SEQ ID NO: 14334); ENST00000636905.1 (SEQ ID NO: 14336); |
| | ENST00000637052.1 (SEQ ID NO: 14338); ENST00000637194.1 (SEQ ID NO: 14340); |
| | ENST00000637490.1 (SEQ ID NO: 14342); ENST00000637587.1 (SEQ ID NO: 14344); |
| | ENST00000637671.1 (SEQ ID NO: 14346); ENST00000637721.1 (SEQ ID NO: 14348); |
| | ENST00000637911.1 (SEQ ID NO: 14350); ENST00000638127.1 (SEQ ID NO: 14352); |
| | ENST00000638142.2 (SEQ ID NO: 14354); ENST00000644458.1 (SEQ ID NO: 14355); |
| | ENST00000645250.1 (SEQ ID NO: 14357); ENST00000646630.1 (SEQ ID NO: 14358) |

Target: SYNJ2

| | |
|---|---|
| pre-mRNA | ENST00000355585.9 (SEQ ID NO: 15842); ENST00000367112.1 (SEQ ID NO: 14360); |
| transcript | ENST00000367113.5 (SEQ ID NO: 15843); ENST00000367122.6 (SEQ ID NO: 14363); |
| | ENST00000449320.2 (SEQ ID NO: 14365); ENST00000485863.1 (SEQ ID NO: 15844); |
| | ENST00000638626.1 (SEQ ID NO: 15845); ENST00000640338.1 (SEQ ID NO: 15846); |
| | ENST00000640569.1 (SEQ ID NO: 14370) |
| transcript | ENST00000355585.9 (SEQ ID NO: 14359); ENST00000367112.1 (SEQ ID NO: 14361); |
| | ENST00000367113.5 (SEQ ID NO: 14362); ENST00000367122.6 (SEQ ID NO: 14364); |
| | ENST00000449320.2 (SEQ ID NO: 14366); ENST00000485863.1 (SEQ ID NO: 14367); |
| | ENST00000638626.1 (SEQ ID NO: 14368); ENST00000640338.1 (SEQ ID NO: 14369); |
| | ENST00000640569.1 (SEQ ID NO: 14371) |

Target: TAZ

| | |
|---|---|
| pre-mRNA | ENST00000369776.8 (SEQ ID NO: 14372); ENST00000426231.5 (SEQ ID NO: 14374); |
| transcript | ENST00000439735.2 (SEQ ID NO: 14376); ENST00000470127.2 (SEQ ID NO: 14378); |
| | ENST00000475699.6 (SEQ ID NO: 14380); ENST00000476679.5 (SEQ ID NO: 14382); |
| | ENST00000476800.2 (SEQ ID NO: 14384); ENST00000479875.1 (SEQ ID NO: 14386); |
| | ENST00000483674.3 (SEQ ID NO: 14388); ENST00000483780.5 (SEQ ID NO: 14390); |
| | ENST00000494912.5 (SEQ ID NO: 14392); ENST00000498029.1 (SEQ ID NO: 14394); |
| | ENST00000601016.6 (SEQ ID NO: 14396); ENST00000612012.5 (SEQ ID NO: 14398); |
| | ENST00000612460.5 (SEQ ID NO: 14400); ENST00000613002.4 (SEQ ID NO: 14402); |
| | ENST00000613634.4 (SEQ ID NO: 14404); ENST00000614595.2 (SEQ ID NO: 14406); |
| | ENST00000615658.5 (SEQ ID NO: 14408); ENST00000615986.4 (SEQ ID NO: 14410); |
| | ENST00000616020.5 (SEQ ID NO: 14412); ENST00000617701.5 (SEQ ID NO: 14414); |
| | ENST00000620808.4 (SEQ ID NO: 14416); ENST00000621647.2 (SEQ ID NO: 14418); |
| | ENST00000651139.1 (SEQ ID NO: 14420); ENST00000652354.1 (SEQ ID NO: 14422); |
| | ENST00000652358.1 (SEQ ID NO: 14424); ENST00000652390.1 (SEQ ID NO: 14426); |
| | ENST00000652476.1 (SEQ ID NO: 14428); ENST00000652644.1 (SEQ ID NO: 14430); |
| | ENST00000652682.1 (SEQ ID NO: 14432); ENST00000652685.1; (SEQ ID NO: 14434) |
| transcript | ENST00000369776.8 (SEQ ID NO: 14373); ENST00000426231.5 (SEQ ID NO: 14375); |
| | ENST00000439735.2 (SEQ ID NO: 14377); ENST00000470127.2 (SEQ ID NO: 14379); |
| | ENST00000475699.6 (SEQ ID NO: 14381); ENST00000476679.5 (SEQ ID NO: 14383); |
| | ENST00000476800.2 (SEQ ID NO: 14385); ENST00000479875.1 (SEQ ID NO: 14387); |
| | ENST00000483674.3 (SEQ ID NO: 14389); ENST00000483780.5 (SEQ ID NO: 14391); |
| | ENST00000494912.5 (SEQ ID NO: 14393); ENST00000498029.1 (SEQ ID NO: 14395); |
| | ENST00000601016.6 (SEQ ID NO: 14397); ENST00000612012.5 (SEQ ID NO: 14399); |
| | ENST00000612460.5 (SEQ ID NO: 14401); ENST00000613002.4 (SEQ ID NO: 14403); |
| | ENST00000613634.4 (SEQ ID NO: 14405); ENST00000614595.2 (SEQ ID NO: 14407); |
| | ENST00000615658.5 (SEQ ID NO: 14409); ENST00000615986.4 (SEQ ID NO: 14411); |
| | ENST00000616020.5 (SEQ ID NO: 14413); ENST00000617701.5 (SEQ ID NO: 14415); |
| | ENST00000620808.4 (SEQ ID NO: 14417); ENST00000621647.2 (SEQ ID NO: 14419); |
| | ENST00000651139.1 (SEQ ID NO: 14421); ENST00000652354.1 (SEQ ID NO: 14423); |
| | ENST00000652358.1 (SEQ ID NO: 14425); ENST00000652390.1 (SEQ ID NO: 14427); |
| | ENST00000652476.1 (SEQ ID NO: 14429); ENST00000652644.1 (SEQ ID NO: 14431); |
| | ENST00000652682.1 (SEQ ID NO: 14433); ENST00000652685.1 (SEQ ID NO: 14435) |

Target: TBL2

| | |
|---|---|
| pre-mRNA | ENST00000305632.11 (SEQ ID NO: 14436); ENST00000417008.5 (SEQ ID NO: 14438); |
| transcript | ENST00000424598.5 (SEQ ID NO: 14440); ENST00000426966.5 (SEQ ID NO: 14442); |
| | ENST00000432538.5 (SEQ ID NO: 14444); ENST00000433464.5 (SEQ ID NO: 14446); |
| | ENST00000435792.5 (SEQ ID NO: 14448); ENST00000437521.5 (SEQ ID NO: 14450); |
| | ENST00000450285.5 (SEQ ID NO: 14452); ENST00000452125.5 (SEQ ID NO: 14454); |
| | ENST00000452475.5 (SEQ ID NO: 14456); ENST00000458466.1 (SEQ ID NO: 14458); |
| | ENST00000459913.5 (SEQ ID NO: 14460); ENST00000465279.5 (SEQ ID NO: 14462); |
| | ENST00000468669.5 (SEQ ID NO: 14464); ENST00000469518.1 (SEQ ID NO: 14466); |
| | ENST00000476136.5 (SEQ ID NO: 14468); ENST00000479892.5 (SEQ ID NO: 14470); |
| | ENST00000488915.1 (SEQ ID NO: 14472); ENST00000495885.1 (SEQ ID NO: 14474); |
| | ENST00000496056.5 (SEQ ID NO: 14476); ENST00000610724.4 (SEQ ID NO: 14478) |
| transcript | ENST00000305632.11 (SEQ ID NO: 14437); ENST00000417008.5 (SEQ ID NO: 14439); |
| | ENST00000424598.5 (SEQ ID NO: 14441); ENST00000426966.5 (SEQ ID NO: 14443); |
| | ENST00000432538.5 (SEQ ID NO: 14445); ENST00000433464.5 (SEQ ID NO: 14447); |
| | ENST00000435792.5 (SEQ ID NO: 14449); ENST00000437521.5 (SEQ ID NO: 14451); |
| | ENST00000450285.5 (SEQ ID NO: 14453); ENST00000452125.5 (SEQ ID NO: 14455); |
| | ENST00000452475.5 (SEQ ID NO: 14457); ENST00000458466.1 (SEQ ID NO: 14459); |
| | ENST00000459913.5 (SEQ ID NO: 14461); ENST00000465279.5 (SEQ ID NO: 14463); |
| | ENST00000468669.5 (SEQ ID NO: 14465); ENST00000469518.1 (SEQ ID NO: 14467); |
| | ENST00000476136.5 (SEQ ID NO: 14469); ENST00000479892.5 (SEQ ID NO: 14471); |
| | ENST00000488915.1 (SEQ ID NO: 14473); ENST00000495885.1 (SEQ ID NO: 14475); |
| | ENST00000496056.5 (SEQ ID NO: 14477); ENST00000610724.4 (SEQ ID NO: 14479) |

Target: TDRD7

| | |
|---|---|
| pre-mRNA | ENST00000355295.5 (SEQ ID NO: 15847); ENST00000492428.1 (SEQ ID NO: 14481) |
| transcript | |
| transcript | ENST00000355295.5 (SEQ ID NO: 14480); ENST00000492428.1 (SEQ ID NO: 14482) |

Target: TECPR2

| | |
|---|---|
| pre-mRNA | ENST00000359520.12 (SEQ ID NO: 15848); ENST00000557786.1 (SEQ ID NO: 14484); |
| transcript | ENST00000558678.1 (SEQ ID NO: 15849); ENST00000559124.1 (SEQ ID NO: 14487); |
| | ENST00000560060.5 (SEQ ID NO: 14489); ENST00000561099.1 (SEQ ID NO: 14491); |
| | ENST00000561228.1 (SEQ ID NO: 15850) |
| transcript | ENST00000359520.12 (SEQ ID NO: 14483); ENST00000557786.1 (SEQ ID NO: 14485); |
| | ENST00000558678.1 (SEQ ID NO: 14486); ENST00000559124.1 (SEQ ID NO: 14488); |
| | ENST00000560060.5 (SEQ ID NO: 14490); ENST00000561099.1 (SEQ ID NO: 14492); |
| | ENST00000561228.1 (SEQ ID NO: 14493) |

Target: TFB1M

| | |
|---|---|
| pre-mRNA | ENST00000367166.5 (SEQ ID NO: 15851); ENST00000466349.1 (SEQ ID NO: 14495); |
| transcript | ENST00000468889.5 (SEQ ID NO: 15852); ENST00000470239.1 (SEQ ID NO: 15853); |
| | ENST00000475849.1 (SEQ ID NO: 14499); ENST00000480390.1 (SEQ ID NO: 14501); |
| | ENST00000487586.5 (SEQ ID NO: 14503); ENST00000489874.5 (SEQ ID NO: 15854); |
| | ENST00000495806.1 (SEQ ID NO: 14506) |
| transcript | ENST00000367166.5 (SEQ ID NO: 14494); ENST00000466349.1 (SEQ ID NO: 14496); |
| | ENST00000468889.5 (SEQ ID NO: 14497); ENST00000470239.1 (SEQ ID NO: 14498); |
| | ENST00000475849.1 (SEQ ID NO: 14500); ENST00000480390.1 (SEQ ID NO: 14502); |
| | ENST00000487586.5 (SEQ ID NO: 14504); ENST00000489874.5 (SEQ ID NO: 14505); |
| | ENST00000495806.1 (SEQ ID NO: 14507) |

Target: TM6SF1

| | |
|---|---|
| pre-mRNA | ENST00000258909.13 (SEQ ID NO: 14508); ENST00000322019.14 (SEQ ID NO: 14510); |
| transcript | ENST00000379384.9 (SEQ ID NO: 14512); ENST00000379390.10 (SEQ ID NO: 14514); |
| | ENST00000561551.1 (SEQ ID NO: 14516); ENST00000564988.5 (SEQ ID NO: 14518); |
| | ENST00000565774.5 (SEQ ID NO: 14520); ENST00000565982.1 (SEQ ID NO: 14522) |
| transcript | ENST00000258909.13 (SEQ ID NO: 14509); ENST00000322019.14 (SEQ ID NO: 14511); |
| | ENST00000379384.9 (SEQ ID NO: 14513); ENST00000379390.10 (SEQ ID NO: 14515); |
| | ENST00000561551.1 (SEQ ID NO: 14517); ENST00000564988.5 (SEQ ID NO: 14519); |
| | ENST00000565774.5 (SEQ ID NO: 14521); ENST00000565982.1 (SEQ ID NO: 14523) |

Target: TNK2

| | |
|---|---|
| pre-mRNA | ENST00000333602.13 (SEQ ID NO: 15855); ENST00000381916.7 (SEQ ID NO: 15856); |
| transcript | ENST00000428187.7 (SEQ ID NO: 14526); ENST00000439230.6 (SEQ ID NO: 14528); |
| | ENST00000671726.1 (SEQ ID NO: 14530); ENST00000671734.1 (SEQ ID NO: 14532); |
| | ENST00000671753.1 (SEQ ID NO: 15857); ENST00000671767.1 (SEQ ID NO: 14535); |
| | ENST00000671831.1 (SEQ ID NO: 14537); ENST00000671880.1 (SEQ ID NO: 14539); |
| | ENST00000672024.1 (SEQ ID NO: 15858); ENST00000672098.1 (SEQ ID NO: 14542); |
| | ENST00000672145.1 (SEQ ID NO: 14544); ENST00000672320.1 (SEQ ID NO: 14546); |
| | ENST00000672542.1 (SEQ ID NO: 14548); ENST00000672548.1 (SEQ ID NO: 14550); |
| | ENST00000672614.1 (SEQ ID NO: 14552); ENST00000672623.1 (SEQ ID NO: 14554); |
| | ENST00000672669.1 (SEQ ID NO: 14556); ENST00000672886.1 (SEQ ID NO: 14558); |
| | ENST00000672887.1 (SEQ ID NO: 15859); ENST00000673038.1 (SEQ ID NO: 14561); |
| | ENST00000673167.1 (SEQ ID NO: 15860); ENST00000673236.1 (SEQ ID NO: 14564); |
| | ENST00000673358.1 (SEQ ID NO: 14566); ENST00000673374.1 (SEQ ID NO: 14568); |
| | ENST00000673420.1 (SEQ ID NO: 15861); ENST00000673440.1 (SEQ ID NO: 14571); |
| | ENST00000673443.1 (SEQ ID NO: 14573); ENST00000673559.1 (SEQ ID NO: 14575) |
| transcript | ENST00000333602.13 (SEQ ID NO: 14524); ENST00000381916.7 (SEQ ID NO: 14525); |
| | ENST00000428187.7 (SEQ ID NO: 14527); ENST00000439230.6 (SEQ ID NO: 14529); |
| | ENST00000671726.1 (SEQ ID NO: 14531); ENST00000671734.1 (SEQ ID NO: 14533); |
| | ENST00000671753.1 (SEQ ID NO: 14534); ENST00000671767.1 (SEQ ID NO: 14536); |
| | ENST00000671831.1 (SEQ ID NO: 14538); ENST00000671880.1 (SEQ ID NO: 14540); |
| | ENST00000672024.1 (SEQ ID NO: 14541); ENST00000672098.1 (SEQ ID NO: 14543); |
| | ENST00000672145.1 (SEQ ID NO: 14545); ENST00000672320.1 (SEQ ID NO: 14547); |
| | ENST00000672542.1 (SEQ ID NO: 14549); ENST00000672548.1 (SEQ ID NO: 14551); |
| | ENST00000672614.1 (SEQ ID NO: 14553); ENST00000672623.1 (SEQ ID NO: 14555); |
| | ENST00000672669.1 (SEQ ID NO: 14557); ENST00000672886.1 (SEQ ID NO: 14559); |
| | ENST00000672887.1 (SEQ ID NO: 14560); ENST00000673038.1 (SEQ ID NO: 14562); |
| | ENST00000673167.1 (SEQ ID NO: 14563); ENST00000673236.1 (SEQ ID NO: 14565); |
| | ENST00000673358.1 (SEQ ID NO: 14567); ENST00000673374.1 (SEQ ID NO: 14569); |
| | ENST00000673420.1 (SEQ ID NO: 14570); ENST00000673440.1 (SEQ ID NO: 14572); |
| | ENST00000673443.1 (SEQ ID NO: 14574); ENST00000673559.1 (SEQ ID NO: 14576) |

Target: TOE1

| | |
|---|---|
| pre-mRNA | ENST00000372090.6 (SEQ ID NO: 14577); ENST00000460057.1 (SEQ ID NO: 14579); |
| transcript | ENST00000471337.5 (SEQ ID NO: 14581); ENST00000477731.5 (SEQ ID NO: 14583); |
| | ENST00000495703.5 (SEQ ID NO: 14585) |
| transcript | ENST00000372090.6 (SEQ ID NO: 14578); ENST00000460057.1 (SEQ ID NO: 14580); |
| | ENST00000471337.5 (SEQ ID NO: 14582); ENST00000477731.5 (SEQ ID NO: 14584); |
| | ENST00000495703.5 (SEQ ID NO: 14586) |

Target: TOR1B

| | |
|---|---|
| pre-mRNA | ENST00000259339.7 (SEQ ID NO: 14587); ENST00000427860.1 (SEQ ID NO: 14589); |
| transcript | ENST00000486372.1 (SEQ ID NO: 14591); ENST00000488169.1; (SEQ ID NO: 14593) |
| transcript | ENST00000259339.7 (SEQ ID NO: 14588); ENST00000427860.1 (SEQ ID NO: 14590); |
| | ENST00000486372.1 (SEQ ID NO: 14592); ENST00000488169.1 (SEQ ID NO: 14594) |

Target: TRAPPC6B

| | |
|---|---|
| pre-mRNA | ENST00000330149.10 (SEQ ID NO: 14595); ENST00000347691.9 (SEQ ID NO: 14597); |
| transcript | ENST00000469361.5 (SEQ ID NO: 14599); ENST00000554018.1 (SEQ ID NO: 14601); |
| | ENST00000555269.5 (SEQ ID NO: 14603); ENST00000556765.1 (SEQ ID NO: 14605); |
| | ENST00000557764.5 (SEQ ID NO: 14607) |
| transcript | ENST00000330149.10 (SEQ ID NO: 14596); ENST00000347691.9 (SEQ ID NO: 14598); |
| | ENST00000469361.5 (SEQ ID NO: 14600); ENST00000554018.1 (SEQ ID NO: 14602); |
| | ENST00000555269.5 (SEQ ID NO: 14604); ENST00000556765.1 (SEQ ID NO: 14606); |
| | ENST00000557764.5 (SEQ ID NO: 14608) |

Target: TRIP4

| | |
|---|---|
| pre-mRNA | ENST00000261884.8 (SEQ ID NO: 15862); ENST00000557834.5 (SEQ ID NO: 14610); |
| transcript | ENST00000558162.1 (SEQ ID NO: 14612); ENST00000558442.1 (SEQ ID NO: 14614); |
| | ENST00000558820.5 (SEQ ID NO: 14616); ENST00000559565.5 (SEQ ID NO: 14618); |
| | ENST00000559777.1 (SEQ ID NO: 14620); ENST00000559833.5 (SEQ ID NO: 14622); |
| | ENST00000560475.1 (SEQ ID NO: 15863); ENST00000560567.5 (SEQ ID NO: 15864); |
| | ENST00000560920.6 (SEQ ID NO: 14626); ENST00000561265.1 (SEQ ID NO: 14628) |
| transcript | ENST00000261884.8 (SEQ ID NO: 14609); ENST00000557834.5 (SEQ ID NO: 14611); |
| | ENST00000558162.1 (SEQ ID NO: 14613); ENST00000558442.1 (SEQ ID NO: 14615); |
| | ENST00000558820.5 (SEQ ID NO: 14617); ENST00000559565.5 (SEQ ID NO: 14619); |
| | ENST00000559777.1 (SEQ ID NO: 14621); ENST00000559833.5 (SEQ ID NO: 14623); |
| | ENST00000560475.1 (SEQ ID NO: 14624); ENST00000560567.5 (SEQ ID NO: 14625); |
| | ENST00000560920.6 (SEQ ID NO: 14627); ENST00000561265.1 (SEQ ID NO: 14629) |

Target: TRMT2A

| | |
|---|---|
| pre-mRNA | ENST00000252136.12 (SEQ ID NO: 14630); ENST00000403707.7 (SEQ ID NO: 14632); |
| transcript | ENST00000404751.7 (SEQ ID NO: 14634); ENST00000439169.2 (SEQ ID NO: 14636); |
| | ENST00000444256.1 (SEQ ID NO: 14638); ENST00000444845.5 (SEQ ID NO: 14640); |
| | ENST00000445045.1 (SEQ ID NO: 14642); ENST00000459644.1 (SEQ ID NO: 14644); |
| | ENST00000463710.1 (SEQ ID NO: 14646); ENST00000464535.1 (SEQ ID NO: 14648); |
| | ENST00000468917.1 (SEQ ID NO: 14650); ENST00000471040.5 (SEQ ID NO: 14652); |
| | ENST00000480339.1 (SEQ ID NO: 14654); ENST00000480460.1 (SEQ ID NO: 14656); |
| | ENST00000487378.1 (SEQ ID NO: 14658); ENST00000487668.5 (SEQ ID NO: 14660); |
| | ENST00000488335.1 (SEQ ID NO: 14662); ENST00000492988.5 (SEQ ID NO: 14664); |
| | ENST00000494641.1 (SEQ ID NO: 14666); ENST00000494820.5 (SEQ ID NO: 14668) |
| transcript | ENST00000252136.12 (SEQ ID NO: 14631); ENST00000403707.7 (SEQ ID NO: 14633); |
| | ENST00000404751.7 (SEQ ID NO: 14635); ENST00000439169.2 (SEQ ID NO: 14637); |
| | ENST00000444256.1 (SEQ ID NO: 14639); ENST00000444845.5 (SEQ ID NO: 14641); |
| | ENST00000445045.1 (SEQ ID NO: 14643); ENST00000459644.1 (SEQ ID NO: 14645); |
| | ENST00000463710.1 (SEQ ID NO: 14647); ENST00000464535.1 (SEQ ID NO: 14649); |
| | ENST00000468917.1 (SEQ ID NO: 14651); ENST00000471040.5 (SEQ ID NO: 14653); |
| | ENST00000480339.1 (SEQ ID NO: 14655); ENST00000480460.1 (SEQ ID NO: 14657); |
| | ENST00000487378.1 (SEQ ID NO: 14659); ENST00000487668.5 (SEQ ID NO: 14661); |
| | ENST00000488335.1 (SEQ ID NO: 14663); ENST00000492988.5 (SEQ ID NO: 14665); |
| | ENST00000494641.1 (SEQ ID NO: 14667); ENST00000494820.5 (SEQ ID NO: 14669) |

Target: TRMU

| | |
|---|---|
| pre-mRNA | ENST00000381019.3 (SEQ ID NO: 14670); ENST00000381021.7 (SEQ ID NO: 14672); |
| transcript | ENST00000441818.5 (SEQ ID NO: 14674); ENST00000453630.5 (SEQ ID NO: 14676); |
| | ENST00000456595.5 (SEQ ID NO: 14678); ENST00000457572.5 (SEQ ID NO: 14680); |
| | ENST00000463785.1 (SEQ ID NO: 14682); ENST00000465378.6 (SEQ ID NO: 14684); |
| | ENST00000470831.1 (SEQ ID NO: 14686); ENST00000476901.1 (SEQ ID NO: 14688); |
| | ENST00000479648.1 (SEQ ID NO: 14690); ENST00000485175.5 (SEQ ID NO: 14692); |
| | ENST00000485559.1 (SEQ ID NO: 14694); ENST00000486620.5 (SEQ ID NO: 14696); |
| | ENST00000491612.1 (SEQ ID NO: 14698); ENST00000493556.2 (SEQ ID NO: 14700); |
| | ENST00000496831.5 (SEQ ID NO: 14702); ENST00000642562.1 (SEQ ID NO: 14704); |
| | ENST00000642923.1 (SEQ ID NO: 14706); ENST00000643137.1 (SEQ ID NO: 14708); |
| | ENST00000644006.1 (SEQ ID NO: 14710); ENST00000645026.1 (SEQ ID NO: 14712); |
| | ENST00000645190.1 (SEQ ID NO: 14714); ENST00000647301.1 (SEQ ID NO: 14716) |
| transcript | ENST00000381019.3 (SEQ ID NO: 14671); ENST00000381021.7 (SEQ ID NO: 14673); |
| | ENST00000441818.5 (SEQ ID NO: 14675); ENST00000453630.5 (SEQ ID NO: 14677); |
| | ENST00000456595.5 (SEQ ID NO: 14679); ENST00000457572.5 (SEQ ID NO: 14681); |
| | ENST00000463785.1 (SEQ ID NO: 14683); ENST00000465378.6 (SEQ ID NO: 14685); |
| | ENST00000470831.1 (SEQ ID NO: 14687); ENST00000476901.1 (SEQ ID NO: 14689); |
| | ENST00000479648.1 (SEQ ID NO: 14691); ENST00000485175.5 (SEQ ID NO: 14693); |
| | ENST00000485559.1 (SEQ ID NO: 14695); ENST00000486620.5 (SEQ ID NO: 14697); |
| | ENST00000491612.1 (SEQ ID NO: 14699); ENST00000493556.2 (SEQ ID NO: 14701); |
| | ENST00000496831.5 (SEQ ID NO: 14703); ENST00000642562.1 (SEQ ID NO: 14705); |
| | ENST00000642923.1 (SEQ ID NO: 14707); ENST00000643137.1 (SEQ ID NO: 14709); |
| | ENST00000644006.1 (SEQ ID NO: 14711); ENST00000645026.1 (SEQ ID NO: 14713); |
| | ENST00000645190.1 (SEQ ID NO: 14715); ENST00000647301.1 (SEQ ID NO: 14717) |

Target: TRPV4

| | |
|---|---|
| pre-mRNA | ENST00000261740.7 (SEQ ID NO: 15865); ENST00000418703.6 (SEQ ID NO: 15866); |
| transcript | ENST00000536570.1 (SEQ ID NO: 14720); ENST00000536838.1 (SEQ ID NO: 15867); |
| | ENST00000537083.5 (SEQ ID NO: 15868); ENST00000538125.5 (SEQ ID NO: 15869); |
| | ENST00000541794.5 (SEQ ID NO: 15870); ENST00000544971.5 (SEQ ID NO: 15871) |
| transcript | ENST00000261740.7 (SEQ ID NO: 14718); ENST00000418703.6 (SEQ ID NO: 14719); |
| | ENST00000536570.1 (SEQ ID NO: 14721); ENST00000536838.1 (SEQ ID NO: 14722); |
| | ENST00000537083.5 (SEQ ID NO: 14723); ENST00000538125.5 (SEQ ID NO: 14724); |
| | ENST00000541794.5 (SEQ ID NO: 14725); ENST00000544971.5 (SEQ ID NO: 14726) |

Target: TTC19

| | |
|---|---|
| pre-mRNA | ENST00000261647.10 (SEQ ID NO: 14727); ENST00000465567.1 (SEQ ID NO: 14729); |
| transcript | ENST00000466729.5 (SEQ ID NO: 14731); ENST00000470399.1 (SEQ ID NO: 14733); |
| | ENST00000470649.1 (SEQ ID NO: 14735); ENST00000475723.5 (SEQ ID NO: 14737); |
| | ENST00000481107.1 (SEQ ID NO: 14739); ENST00000497842.6 (SEQ ID NO: 14741); |
| | ENST00000578103.1 (SEQ ID NO: 14743); ENST00000583704.1 (SEQ ID NO: 14745) |
| transcript | ENST00000261647.10 (SEQ ID NO: 14728); ENST00000465567.1 (SEQ ID NO: 14730); |
| | ENST00000466729.5 (SEQ ID NO: 14732); ENST00000470399.1 (SEQ ID NO: 14734); |
| | ENST00000470649.1 (SEQ ID NO: 14736); ENST00000475723.5 (SEQ ID NO: 14738); |
| | ENST00000481107.1 (SEQ ID NO: 14740); ENST00000497842.6 (SEQ ID NO: 14742); |
| | ENST00000578103.1 (SEQ ID NO: 14744); ENST00000583704.1 (SEQ ID NO: 14746) |

Target: TYMP

| | |
|---|---|
| pre-mRNA | ENST00000252029.8 (SEQ ID NO: 14747); ENST00000395678.7 (SEQ ID NO: 14749); |
| transcript | ENST00000395680.6 (SEQ ID NO: 14751); ENST00000395681.6 (SEQ ID NO: 14753); |
| | ENST00000425169.1 (SEQ ID NO: 14755); ENST00000476284.1 (SEQ ID NO: 14757); |
| | ENST00000487162.1 (SEQ ID NO: 14759); ENST00000487577.5 (SEQ ID NO: 14761); |
| | ENST00000650719.1 (SEQ ID NO: 14763); ENST00000651095.1 (SEQ ID NO: 14765); |
| | ENST00000651196.1 (SEQ ID NO: 14767); ENST00000651401.1 (SEQ ID NO: 14769); |
| | ENST00000651490.1 (SEQ ID NO: 14771); ENST00000651906.1 (SEQ ID NO: 14773); |
| | ENST00000652237.1 (SEQ ID NO: 14775); ENST00000652352.1 (SEQ ID NO: 14777); |
| | ENST00000652401.1 (SEQ ID NO: 14779) |
| transcript | ENST00000252029.8 (SEQ ID NO: 14748); ENST00000395678.7 (SEQ ID NO: 14750); |
| | ENST00000395680.6 (SEQ ID NO: 14752); ENST00000395681.6 (SEQ ID NO: 14754); |
| | ENST00000425169.1 (SEQ ID NO: 14756); ENST00000476284.1 (SEQ ID NO: 14758); |
| | ENST00000487162.1 (SEQ ID NO: 14760); ENST00000487577.5 (SEQ ID NO: 14762); |
| | ENST00000650719.1 (SEQ ID NO: 14764); ENST00000651095.1 (SEQ ID NO: 14766); |
| | ENST00000651196.1 (SEQ ID NO: 14768); ENST00000651401.1 (SEQ ID NO: 14770); |
| | ENST00000651490.1 (SEQ ID NO: 14772); ENST00000651906.1 (SEQ ID NO: 14774); |
| | ENST00000652237.1 (SEQ ID NO: 14776); ENST00000652352.1 (SEQ ID NO: 14778); |
| | ENST00000652401.1 (SEQ ID NO: 14780) |

Target: UMPS

| | |
|---|---|
| pre-mRNA | ENST00000232607.7 (SEQ ID NO: 14781); ENST00000460034.5 (SEQ ID NO: 14783); |
| transcript | ENST00000462091.5 (SEQ ID NO: 14785); ENST00000467167.5 (SEQ ID NO: 14787); |
| | ENST00000474588.5 (SEQ ID NO: 14789); ENST00000479719.5 (SEQ ID NO: 14791); |
| | ENST00000487622.5 (SEQ ID NO: 14793); ENST00000495751.1 (SEQ ID NO: 14795); |
| | ENST00000497791.5 (SEQ ID NO: 14797); ENST00000498715.1 (SEQ ID NO: 14799); |
| | ENST00000628619.1 (SEQ ID NO: 14801) |
| transcript | ENST00000232607.7 (SEQ ID NO: 14782); ENST00000460034.5 (SEQ ID NO: 14784); |
| | ENST00000462091.5 (SEQ ID NO: 14786); ENST00000467167.5 (SEQ ID NO: 14788); |
| | ENST00000474588.5 (SEQ ID NO: 14790); ENST00000479719.5 (SEQ ID NO: 14792); |
| | ENST00000487622.5 (SEQ ID NO: 14794); ENST00000495751.1 (SEQ ID NO: 14796); |
| | ENST00000497791.5 (SEQ ID NO: 14798); ENST00000498715.1 (SEQ ID NO: 14800); |
| | ENST00000628619.1 (SEQ ID NO: 14802) |

Target: UROD

| | |
|---|---|
| pre-mRNA | ENST00000246337.9 (SEQ ID NO: 14803); ENST00000428106.1 (SEQ ID NO: 14805); |
| transcript | ENST00000434478.6 (SEQ ID NO: 14807); ENST00000460334.5 (SEQ ID NO: 14809); |
| | ENST00000460906.5 (SEQ ID NO: 14811); ENST00000461035.5 (SEQ ID NO: 14813); |
| | ENST00000462688.5 (SEQ ID NO: 14815); ENST00000463092.5 (SEQ ID NO: 14817); |
| | ENST00000465678.1 (SEQ ID NO: 14819); ENST00000466193.1 (SEQ ID NO: 14821); |
| | ENST00000469548.5 (SEQ ID NO: 14823); ENST00000472254.1 (SEQ ID NO: 14825); |
| | ENST00000473012.1 (SEQ ID NO: 14827); ENST00000478467.5 (SEQ ID NO: 14829); |
| | ENST00000486699.5 (SEQ ID NO: 14831); ENST00000490385.5 (SEQ ID NO: 14833); |
| | ENST00000491300.5 (SEQ ID NO: 14835); ENST00000491773.6 (SEQ ID NO: 14837); |
| | ENST00000494399.5 (SEQ ID NO: 14839); ENST00000496439.1 (SEQ ID NO: 14841); |
| | ENST00000636293.1 (SEQ ID NO: 14843); ENST00000636836.1 (SEQ ID NO: 14845); |
| | ENST00000650713.1 (SEQ ID NO: 14847); ENST00000651476.1 (SEQ ID NO: 14849); |
| | ENST00000652165.1 (SEQ ID NO: 14851); ENST00000652287.1 (SEQ ID NO: 14853); |
| | ENST00000652514.1 (SEQ ID NO: 14855) |
| transcript | ENST00000246337.9 (SEQ ID NO: 14804); ENST00000428106.1 (SEQ ID NO: 14806); |
| | ENST00000434478.6 (SEQ ID NO: 14808); ENST00000460334.5 (SEQ ID NO: 14810); |
| | ENST00000460906.5 (SEQ ID NO: 14812); ENST00000461035.5 (SEQ ID NO: 14814); |
| | ENST00000462688.5 (SEQ ID NO: 14816); ENST00000463092.5 (SEQ ID NO: 14818); |
| | ENST00000465678.1 (SEQ ID NO: 14820); ENST00000466193.1 (SEQ ID NO: 14822); |
| | ENST00000469548.5 (SEQ ID NO: 14824); ENST00000472254.1 (SEQ ID NO: 14826); |
| | ENST00000473012.1 (SEQ ID NO: 14828); ENST00000478467.5 (SEQ ID NO: 14830); |
| | ENST00000486699.5 (SEQ ID NO: 14832); ENST00000490385.5 (SEQ ID NO: 14834); |
| | ENST00000491300.5 (SEQ ID NO: 14836); ENST00000491773.6 (SEQ ID NO: 14838); |
| | ENST00000494399.5 (SEQ ID NO: 14840); ENST00000496439.1 (SEQ ID NO: 14842); |
| | ENST00000636293.1 (SEQ ID NO: 14844); ENST00000636836.1 (SEQ ID NO: 14846); |
| | ENST00000650713.1 (SEQ ID NO: 14848); ENST00000651476.1 (SEQ ID NO: 14850); |
| | ENST00000652165.1 (SEQ ID NO: 14852); ENST00000652287.1 (SEQ ID NO: 14854); |
| | ENST00000652514.1 (SEQ ID NO: 14856) |

Target: WDR11

| | |
|---|---|
| pre-mRNA | ENST00000263461.11 (SEQ ID NO: 15872); ENST00000462529.2 (SEQ ID NO: 14858); |
| transcript | ENST00000470052.5 (SEQ ID NO: 14860); ENST00000478567.5 (SEQ ID NO: 14862); |
| | ENST00000497136.6 (SEQ ID NO: 15873); ENST00000603658.1 (SEQ ID NO: 14865); |
| | ENST00000604220.5 (SEQ ID NO: 14867); ENST00000604509.5 (SEQ ID NO: 15874); |
| | ENST00000604585.5 (SEQ ID NO: 14870); ENST00000604714.1 (SEQ ID NO: 14872); |
| | ENST00000605069.5 (SEQ ID NO: 14874); ENST00000605178.5 (SEQ ID NO: 14876); |
| | ENST00000605202.5 (SEQ ID NO: 14878); ENST00000605320.1 (SEQ ID NO: 14880); |
| | ENST00000605376.5 (SEQ ID NO: 14882); ENST00000605543.5 (SEQ ID NO: 15875); |
| | ENST00000605659.1 (SEQ ID NO: 14885) |
| transcript | ENST00000263461.11 (SEQ ID NO: 14857); ENST00000462529.2 (SEQ ID NO: 14859); |
| | ENST00000470052.5 (SEQ ID NO: 14861); ENST00000478567.5 (SEQ ID NO: 14863); |
| | ENST00000497136.6 (SEQ ID NO: 14864); ENST00000603658.1 (SEQ ID NO: 14866); |
| | ENST00000604220.5 (SEQ ID NO: 14868); ENST00000604509.5 (SEQ ID NO: 14869); |
| | ENST00000604585.5 (SEQ ID NO: 14871); ENST00000604714.1 (SEQ ID NO: 14873); |
| | ENST00000605069.5 (SEQ ID NO: 14875); ENST00000605178.5 (SEQ ID NO: 14877); |
| | ENST00000605202.5 (SEQ ID NO: 14879); ENST00000605320.1 (SEQ ID NO: 14881); |
| | ENST00000605376.5 (SEQ ID NO: 14883); ENST00000605543.5 (SEQ ID NO: 14884); |
| | ENST00000605659.1 (SEQ ID NO: 14886) |

Target: WDR62

| | |
|---|---|
| pre-mRNA | ENST00000270301.11 (SEQ ID NO: 15876); ENST00000378860.8 (SEQ ID NO: 14888); |
| transcript | ENST00000401500.7 (SEQ ID NO: 15877); ENST00000427823.2 (SEQ ID NO: 14891); |
| | ENST00000587391.5 (SEQ ID NO: 15878); ENST00000589953.1 (SEQ ID NO: 14894); |
| | ENST00000608676.1 (SEQ ID NO: 14896); ENST00000644764.1 (SEQ ID NO: 14898) |
| transcript | ENST00000270301.11 (SEQ ID NO: 14887); ENST00000378860.8 (SEQ ID NO: 14889); |
| | ENST00000401500.7 (SEQ ID NO: 14890); ENST00000427823.2 (SEQ ID NO: 14892); |
| | ENST00000587391.5 (SEQ ID NO: 14893); ENST00000589953.1 (SEQ ID NO: 14895); |
| | ENST00000608676.1 (SEQ ID NO: 14897); ENST00000644764.1 (SEQ ID NO: 14899) |

Target: WIPI1

| | |
|---|---|
| pre-mRNA | ENST00000262139.10 (SEQ ID NO: 15879); ENST00000546360.5 (SEQ ID NO: 15880); |
| transcript | ENST00000585393.1 (SEQ ID NO: 14902); ENST00000586815.1 (SEQ ID NO: 14904); |
| | ENST00000587731.1 (SEQ ID NO: 14906); ENST00000589316.5 (SEQ ID NO: 15881); |
| | ENST00000589459.5 (SEQ ID NO: 15882); ENST00000590402.1 (SEQ ID NO: 14910); |
| | ENST00000591494.5 (SEQ ID NO: 14912); ENST00000591744.1 (SEQ ID NO: 14914); |
| | ENST00000592645.5 (SEQ ID NO: 14916) |
| transcript | ENST00000262139.10 (SEQ ID NO: 14900); ENST00000546360.5 (SEQ ID NO: 14901); |
| | ENST00000585393.1 (SEQ ID NO: 14903); ENST00000586815.1 (SEQ ID NO: 14905); |
| | ENST00000587731.1 (SEQ ID NO: 14907); ENST00000589316.5 (SEQ ID NO: 14908); |
| | ENST00000589459.5 (SEQ ID NO: 14909); ENST00000590402.1 (SEQ ID NO: 14911); |
| | ENST00000591494.5 (SEQ ID NO: 14913); ENST00000591744.1 (SEQ ID NO: 14915); |
| | ENST00000592645.5 (SEQ ID NO: 14917) |

Target: WRAP53

| | |
|---|---|
| pre-mRNA | ENST00000316024.9 (SEQ ID NO: 14918); ENST00000396463.6 (SEQ ID NO: 14920); |
| transcript | ENST00000431639.6 (SEQ ID NO: 14922); ENST00000457584.6 (SEQ ID NO: 14924); |
| | ENST00000463804.6 (SEQ ID NO: 14926); ENST00000467699.5 (SEQ ID NO: 14928); |
| | ENST00000471973.6 (SEQ ID NO: 14930); ENST00000498114.1 (SEQ ID NO: 14932); |
| | ENST00000498311.5 (SEQ ID NO: 14934); ENST00000534050.5 (SEQ ID NO: 14936) |
| transcript | ENST00000316024.9 (SEQ ID NO: 14919); ENST00000396463.6 (SEQ ID NO: 14921); |
| | ENST00000431639.6 (SEQ ID NO: 14923); ENST00000457584.6 (SEQ ID NO: 14925); |
| | ENST00000463804.6 (SEQ ID NO: 14927); ENST00000467699.5 (SEQ ID NO: 14929); |
| | ENST00000471973.6 (SEQ ID NO: 14931); ENST00000498114.1 (SEQ ID NO: 14933); |
| | ENST00000498311.5 (SEQ ID NO: 14935); ENST00000534050.5 (SEQ ID NO: 14937) |

Target: XPO1

| | |
|---|---|
| pre-mRNA | ENST00000401558.7 (SEQ ID NO: 15883); ENST00000404992.6 (SEQ ID NO: 15884); |
| transcript | ENST00000406957.5 (SEQ ID NO: 15885); ENST00000420673.5 (SEQ ID NO: 14941); |
| | ENST00000422552.5 (SEQ ID NO: 14943); ENST00000428210.5 (SEQ ID NO: 15886); |
| | ENST00000436018.1 (SEQ ID NO: 14946); ENST00000437159.5 (SEQ ID NO: 14948); |
| | ENST00000443240.5 (SEQ ID NO: 15887); ENST00000449444.5 (SEQ ID NO: 14951); |
| | ENST00000451765.5 (SEQ ID NO: 15888); ENST00000457483.5 (SEQ ID NO: 14954); |
| | ENST00000460037.1 (SEQ ID NO: 14956); ENST00000461407.5 (SEQ ID NO: 14958); |
| | ENST00000468259.1 (SEQ ID NO: 14960); ENST00000469337.1 (SEQ ID NO: 14962); |
| | ENST00000475744.1 (SEQ ID NO: 14964); ENST00000476585.5 (SEQ ID NO: 14966); |
| | ENST00000481073.5 (SEQ ID NO: 14968); ENST00000481214.1 (SEQ ID NO: 14970); |
| | ENST00000489954.1 (SEQ ID NO: 14972); ENST00000492182.5 (SEQ ID NO: 14974); |
| | ENST00000494468.1 (SEQ ID NO: 14976); ENST00000495003.1 (SEQ ID NO: 14978) |
| transcript | ENST00000401558.7 (SEQ ID NO: 14938); ENST00000404992.6 (SEQ ID NO: 14939); |
| | ENST00000406957.5 (SEQ ID NO: 14940); ENST00000420673.5 (SEQ ID NO: 14942); |
| | ENST00000422552.5 (SEQ ID NO: 14944); ENST00000428210.5 (SEQ ID NO: 14945); |
| | ENST00000436018.1 (SEQ ID NO: 14947); ENST00000437159.5 (SEQ ID NO: 14949); |
| | ENST00000443240.5 (SEQ ID NO: 14950); ENST00000449444.5 (SEQ ID NO: 14952); |
| | ENST00000451765.5 (SEQ ID NO: 14953); ENST00000457483.5 (SEQ ID NO: 14955); |
| | ENST00000460037.1 (SEQ ID NO: 14957); ENST00000461407.5 (SEQ ID NO: 14959); |
| | ENST00000468259.1 (SEQ ID NO: 14961); ENST00000469337.1 (SEQ ID NO: 14963); |
| | ENST00000475744.1 (SEQ ID NO: 14965); ENST00000476585.5 (SEQ ID NO: 14967); |
| | ENST00000481073.5 (SEQ ID NO: 14969); ENST00000481214.1 (SEQ ID NO: 14971); |
| | ENST00000489954.1 (SEQ ID NO: 14973); ENST00000492182.5 (SEQ ID NO: 14975); |
| | ENST00000494468.1 (SEQ ID NO: 14977); ENST00000495003.1 (SEQ ID NO: 14979) |

Target: YY1AP1

| | |
|---|---|
| pre-mRNA | ENST00000295566.8 (SEQ ID NO: 14980); ENST00000311573.9 (SEQ ID NO: 14982); |
| transcript | ENST00000347088.9 (SEQ ID NO: 14984); ENST00000354691.9 (SEQ ID NO: 14986); |
| | ENST00000355499.8 (SEQ ID NO: 14988); ENST00000359205.9 (SEQ ID NO: 14990); |
| | ENST00000361140.8 (SEQ ID NO: 14992); ENST00000361831.9 (SEQ ID NO: 14994); |
| | ENST00000368330.6 (SEQ ID NO: 14996); ENST00000368339.9 (SEQ ID NO: 14998); |
| | ENST00000368340.9 (SEQ ID NO: 15000); ENST00000404643.5 (SEQ ID NO: 15002); |
| | ENST00000405763.7 (SEQ ID NO: 15004); ENST00000407221.5 (SEQ ID NO: 15006); |
| | ENST00000436865.5 (SEQ ID NO: 15008); ENST00000442834.6 (SEQ ID NO: 15010); |
| | ENST00000443231.5 (SEQ ID NO: 15012); ENST00000454523.5 (SEQ ID NO: 15014); |
| | ENST00000466366.1 (SEQ ID NO: 15016); ENST00000476027.5 (SEQ ID NO: 15018); |
| | ENST00000476093.5 (SEQ ID NO: 15020); ENST00000477470.1 (SEQ ID NO: 15022); |
| | ENST00000488784.5 (SEQ ID NO: 15024); ENST00000493625.5 (SEQ ID NO: 15026); |
| | ENST00000496324.5 (SEQ ID NO: 15028) |
| transcript | ENST00000295566.8 (SEQ ID NO: 14981); ENST00000311573.9 (SEQ ID NO: 14983); |
| | ENST00000347088.9 (SEQ ID NO: 14985); ENST00000354691.9 (SEQ ID NO: 14987); |
| | ENST00000355499.8 (SEQ ID NO: 14989); ENST00000359205.9 (SEQ ID NO: 14991); |
| | ENST00000361140.8 (SEQ ID NO: 14993); ENST00000361831.9 (SEQ ID NO: 14995); |
| | ENST00000368330.6 (SEQ ID NO: 14997); ENST00000368339.9 (SEQ ID NO: 14999); |
| | ENST00000368340.9 (SEQ ID NO: 15001); ENST00000404643.5 (SEQ ID NO: 15003); |
| | ENST00000405763.7 (SEQ ID NO: 15005); ENST00000407221.5 (SEQ ID NO: 15007); |
| | ENST00000436865.5 (SEQ ID NO: 15009); ENST00000442834.6 (SEQ ID NO: 15011); |
| | ENST00000443231.5 (SEQ ID NO: 15013); ENST00000454523.5 (SEQ ID NO: 15015); |
| | ENST00000466366.1 (SEQ ID NO: 15017); ENST00000476027.5 (SEQ ID NO: 15019); |
| | ENST00000476093.5 (SEQ ID NO: 15021); ENST00000477470.1 (SEQ ID NO: 15023); |
| | ENST00000488784.5 (SEQ ID NO: 15025); ENST00000493625.5 (SEQ ID NO: 15027); |
| | ENST00000496324.5 (SEQ ID NO: 15029) |

Target: ZC3H14

| | |
|---|---|
| pre-mRNA | ENST00000251038.10 (SEQ ID NO: 15889); ENST00000302216.12 (SEQ ID NO: 15890); |
| transcript | ENST00000318308.10 (SEQ ID NO: 15032); ENST00000336693.8 (SEQ ID NO: 15891); |
| | ENST00000393514.9 (SEQ ID NO: 15892); ENST00000406216.7 (SEQ ID NO: 15036); |
| | ENST00000553495.5 (SEQ ID NO: 15038); ENST00000554020.5 (SEQ ID NO: 15040); |
| | ENST00000554602.5 (SEQ ID NO: 15042); ENST00000555120.1 (SEQ ID NO: 15044); |
| | ENST00000555755.5 (SEQ ID NO: 15893); ENST00000555792.1 (SEQ ID NO: 15047); |
| | ENST00000555799.5 (SEQ ID NO: 15049); ENST00000555851.6 (SEQ ID NO: 15051); |
| | ENST00000555900.5 (SEQ ID NO: 15053); ENST00000556000.5 (SEQ ID NO: 15894); |
| | ENST00000556110.5 (SEQ ID NO: 15056); ENST00000556158.5 (SEQ ID NO: 15058); |
| | ENST00000556945.5 (SEQ ID NO: 15895); ENST00000557047.2 (SEQ ID NO: 15061); |
| | ENST00000557491.1 (SEQ ID NO: 15063); ENST00000557605.1 (SEQ ID NO: 15065); |
| | ENST00000557607.5 (SEQ ID NO: 15896); ENST00000557693.5 (SEQ ID NO: 15068); |
| | ENST00000557737.1 (SEQ ID NO: 15070); ENST00000649731.1 (SEQ ID NO: 15897) |
| transcript | ENST00000251038.10 (SEQ ID NO: 15030); ENST00000302216.12 (SEQ ID NO: 15031); |
| | ENST00000318308.10 (SEQ ID NO: 15033); ENST00000336693.8 (SEQ ID NO: 15034); |
| | ENST00000393514.9 (SEQ ID NO: 15035); ENST00000406216.7 (SEQ ID NO: 15037); |
| | ENST00000553495.5 (SEQ ID NO: 15039); ENST00000554020.5 (SEQ ID NO: 15041); |
| | ENST00000554602.5 (SEQ ID NO: 15043); ENST00000555120.1 (SEQ ID NO: 15045); |
| | ENST00000555755.5 (SEQ ID NO: 15046); ENST00000555792.1 (SEQ ID NO: 15048); |
| | ENST00000555799.5 (SEQ ID NO: 15050); ENST00000555851.6 (SEQ ID NO: 15052); |
| | ENST00000555900.5 (SEQ ID NO: 15054); ENST00000556000.5 (SEQ ID NO: 15055); |
| | ENST00000556110.5 (SEQ ID NO: 15057); ENST00000556158.5 (SEQ ID NO: 15059); |
| | ENST00000556945.5 (SEQ ID NO: 15060); ENST00000557047.2 (SEQ ID NO: 15062); |
| | ENST00000557491.1 (SEQ ID NO: 15064); ENST00000557605.1 (SEQ ID NO: 15066); |
| | ENST00000557607.5 (SEQ ID NO: 15067); ENST00000557693.5 (SEQ ID NO: 15069); |
| | ENST00000557737.1 (SEQ ID NO: 15071); ENST00000649731.1 (SEQ ID NO: 15072) |

Where reference is made to a polynucleic acid polymer sequence, the skilled person will understand that one or more substitutions may be tolerated, optionally two substitutions may be tolerated in the sequence, such that it maintains the ability to hybridize to the target sequence; or where the substitution is in a target sequence, the ability to be recognized as the target sequence. References to sequence identity may be determined by BLAST sequence alignment using standard/default parameters. For example, the sequence may have 99% identity and still function according to the present disclosure. In other embodiments, the sequence may have 98% identity and still function according to the present disclosure. In another embodiment, the sequence may have 95% identity and still function according to the present disclosure. In another embodiment, the sequence may have 90% identity and still function according to the present disclosure.

### Antisense Oligomers

Provided herein is a composition comprising an antisense oligomer that prevents alternative splicing by binding to a targeted portion of a SYNGAP1 pre-mRNA. Provided herein also is a composition comprising an antisense oligomer that prevents alternative splicing by binding to a targeted portion of a target pre-mRNA, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. As used herein, the terms “ASO” and “antisense oligomer” are used interchangeably and refer to an oligomer such as a polynucleotide or polynucleic acid polymer, comprising nucleobases that hybridizes to a target nucleic acid (e.g., a SYNGAP1 pre-mRNA) sequence by Watson-Crick base pairing or wobble base pairing (G-U). The ASO may have exact sequence complementary to the target sequence or near complementarity (e.g., sufficient complementarity to bind the target sequence and enhancing splicing at a splice site). ASOs are designed so that they bind (hybridize) to a target nucleic acid (e.g., a targeted portion of a pre-mRNA transcript) and remain hybridized under physiological conditions. Typically, if they hybridize to a site other than the intended (targeted) nucleic acid sequence, they hybridize to a limited number of sequences that are not a target nucleic acid (to a few sites other than a target nucleic acid). Design of an ASO can take into consideration the occurrence of the nucleic acid sequence of the targeted portion of the pre-mRNA transcript or a sufficiently similar nucleic acid sequence in other locations in the genome or cellular pre-mRNA or transcriptome, such that the likelihood the ASO will bind other sites and cause “off-target” effects is limited. Any antisense oligomers known in the art, for example in PCT Application No. PCT/US2014/054151, published as WO 2015/035091, titled “Reducing Nonsense-Mediated mRNA Decay,” incorporated by reference herein, can be used to practice the methods described herein.

In some embodiments, ASOs “specifically hybridize” to or are “specific” to a target nucleic acid or a targeted portion of a RIC pre-mRNA. Typically, such hybridization occurs with a Tm substantially greater than 37° C., preferably at least 50° C., and typically between 60° C. to approximately 90° C. Such hybridization preferably corresponds to stringent hybridization conditions. At a given ionic strength and pH, the Tm is the temperature at which 50% of a target sequence hybridizes to a complementary oligonucleotide.

Oligomers, such as oligonucleotides, are “complementary” to one another when hybridization occurs in an antiparallel configuration between two single-stranded polynucleotides. A double-stranded polynucleotide can be “complementary” to another polynucleotide, if hybridization can occur between one of the strands of the first polynucleotide and the second. Complementarity (the degree to which one polynucleotide is complementary with another) is quantifiable in terms of the proportion (e.g., the percentage) of bases in opposing strands that are expected to form hydrogen bonds with each other, according to generally accepted base-pairing rules. The sequence of an antisense oligomer (ASO) need not be 100% complementary to that of its target nucleic acid to hybridize. In certain embodiments, ASOs can comprise at least 70%, at least 75%, at least 80%, at least 85%, at least 90%, at least 95%, at least 96%, at least 97%, at least 98%, or at least 99% sequence complementarity to a target region within the target nucleic acid sequence to which they are targeted. For example, an ASO in which 18 of 20 nucleobases of the oligomeric compound are complementary to a target region, and would therefore specifically hybridize, would represent 90 percent complementarity. In this example, the remaining non-complementary nucleobases may be clustered together or interspersed with complementary nucleobases and need not be contiguous to each other or to complementary nucleobases. Percent complementarity of an ASO with a region of a target nucleic acid can be determined routinely using BLAST programs (basic local alignment search tools) and PowerBLAST programs known in the art (Altschul, et al., J. Mol. Biol., 1990, 215, 403-410; Zhang and Madden, Genome Res., 1997, 7, 649-656).

An ASO need not hybridize to all nucleobases in a target sequence and the nucleobases to which it does hybridize may be contiguous or noncontiguous. ASOs may hybridize over one or more segments of a pre-mRNA transcript, such that intervening or adjacent segments are not involved in the hybridization event (e.g., a loop structure or hairpin structure may be formed). In certain embodiments, an ASO hybridizes to noncontiguous nucleobases in a target pre-mRNA transcript. For example, an ASO can hybridize to nucleobases in a pre-mRNA transcript that are separated by one or more nucleobase(s) to which the ASO does not hybridize.

The ASOs described herein comprise nucleobases that are complementary to nucleobases present in a target portion of a NSAE pre-mRNA. The term ASO embodies oligonucleotides and any other oligomeric molecule that comprises nucleobases capable of hybridizing to a complementary nucleobase on a target mRNA but does not comprise a sugar moiety, such as a peptide nucleic acid (PNA). The ASOs may comprise naturally-occurring nucleotides, nucleotide analogs, modified nucleotides, or any combination of two or three of the preceding. The term “naturally occurring nucleotides” includes deoxyribonucleotides and ribonucleotides. The term “modified nucleotides” includes nucleotides with modified or substituted sugar groups and/or having a modified backbone. In some embodiments, all of the nucleotides of the ASO are modified nucleotides. Chemical modifications of ASOs or components of ASOs that are compatible with the methods and compositions described herein will be evident to one of skill in the art and can be found, for example, in U.S. Pat. No. 8,258,109 B2, U.S. Pat. No. 5,656,612, U.S. Patent Publication No. 2012/0190728, and Dias and Stein, Mol. Cancer Ther. 2002, 347-355, herein incorporated by reference in their entirety.

One or more nucleobases of an ASO may be any naturally occurring, unmodified nucleobase such as adenine, guanine, cytosine, thymine and uracil, or any synthetic or modified nucleobase that is sufficiently similar to an unmodified nucleobase such that it is capable of hydrogen bonding with a nucleobase present on a target pre-mRNA. Examples of modified nucleobases include, without limitation, hypoxanthine, xanthine, 7-methylguanine, 5, 6-dihydrouracil, 5-methylcytosine, and 5-hydroxymethoylcytosine.

The ASOs described herein also comprise a backbone structure that connects the components of an oligomer. The term “backbone structure” and “oligomer linkages” may be used interchangeably and refer to the connection between monomers of the ASO. In naturally occurring oligonucleotides, the backbone comprises a 3′-5′ phosphodiester linkage connecting sugar moieties of the oligomer. The backbone structure or oligomer linkages of the ASOs described herein may include (but are not limited to) phosphorothioate, phosphorodithioate, phosphoroselenoate, phosphorodiselenoate, phosphoroanilothioate, phosphoraniladate, phosphoramidate, and the like. See, e.g., LaPlanche, et al., Nucleic Acids Res. 14:9081 (1986); Stec, et al., J. Am. Chem. Soc. 106:6077 (1984), Stein, et al., Nucleic Acids Res. 16:3209 (1988), Zon, et al., Anti Cancer Drug Design 6:539 (1991); Zon, et al., Oligonucleotides and Analogues: A Practical Approach, pp. 87-108 (F. Eckstein, Ed., Oxford University Press, Oxford England (1991)); Stec, et al., U.S. Pat. No. 5,151,510; Uhlmann and Peyman, Chemical Reviews 90:543 (1990). In some embodiments, the backbone structure of the ASO does not contain phosphorous but rather contains peptide bonds, for example in a peptide nucleic acid (PNA), or linking groups including carbamate, amides, and linear and cyclic hydrocarbon groups. In some embodiments, the backbone modification is a phosphothioate linkage. In some embodiments, the backbone modification is a phosphoramidate linkage.

In some embodiments, the stereochemistry at each of the phosphorus intemucleotide linkages of the ASO backbone is random. In some embodiments, the stereochemistry at each of the phosphorus internucleotide linkages of the ASO backbone is controlled and is not random. For example, U.S. Pat. App. Pub. No. 2014/0194610, “Methods for the Synthesis of Functionalized Nucleic Acids,” incorporated herein by reference, describes methods for independently selecting the handedness of chirality at each phosphorous atom in a nucleic acid oligomer. In some embodiments, an ASO used in the methods of the invention, including, but not limited to, any of the ASOs set forth herein in Table 1, comprises an ASO having phosphorus internucleotide linkages that are not random. In some embodiments, a composition used in the methods of the invention comprises a pure diastereomeric ASO. In some embodiments, a composition used in the methods of the invention comprises an ASO that has diastereomeric purity of at least about 90%, at least about 91%, at least about 92%, at least about 93%, at least about 94%, at least about 95%, at least about 96%, at least about 97%, at least about 98%, at least about 99%, about 100%, about 90% to about 100%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

In some embodiments, the ASO has a nonrandom mixture of Rp and Sp configurations at its phosphorus internucleotide linkages. For example, it has been suggested that a mix of Rp and Sp is required in antisense oligonucleotides to achieve a balance between good activity and nuclease stability (Wan, et al., 2014, “Synthesis, biophysical properties and biological activity of second generation antisense oligonucleotides containing chiral phosphorothioate linkages,” Nucleic Acids Research 42(22): 13456-13468, incorporated herein by reference). In some embodiments, an ASO used in the methods of the invention, including, but not limited to, any of the ASOs set forth herein in Table 1, comprises about 5-100% Rp, at least about 5% Rp, at least about 10% Rp, at least about 15% Rp, at least about 20% Rp, at least about 25% Rp, at least about 30% Rp, at least about 35% Rp, at least about 40% Rp, at least about 45% Rp, at least about 50% Rp, at least about 55% Rp, at least about 60% Rp, at least about 65% Rp, at least about 70% Rp, at least about 75% Rp, at least about 80% Rp, at least about 85% Rp, at least about 90% Rp, or at least about 95% Rp, with the remainder Sp, or about 100% Rp. In some embodiments, an ASO used in the methods of the invention, including, but not limited to, any of the ASOs set forth herein in Table 1, comprises about 10% to about 100% Rp, about 15% to about 100% Rp, about 20% to about 100% Rp, about 25% to about 100% Rp, about 30% to about 100% Rp, about 35% to about 100% Rp, about 40% to about 100% Rp, about 45% to about 100% Rp, about 50% to about 100% Rp, about 55% to about 100% Rp, about 60% to about 100% Rp, about 65% to about 100% Rp, about 70% to about 100% Rp, about 75% to about 100% Rp, about 80% to about 100% Rp, about 85% to about 100% Rp, about 90% to about 100% Rp, or about 95% to about 100% Rp, about 20% to about 80% Rp, about 25% to about 75% Rp, about 30% to about 70% Rp, about 40% to about 60% Rp, or about 45% to about 55% Rp, with the remainder Sp.

In embodiments, an ASO used in the methods of the invention, including, but not limited to, any of the ASOs set forth herein in Table 1, comprises about 5-100% Sp, at least about 5% Sp, at least about 10% Sp, at least about 15% Sp, at least about 20% Sp, at least about 25% Sp, at least about 30% Sp, at least about 35% Sp, at least about 40% Sp, at least about 45% Sp, at least about 50% Sp, at least about 55% Sp, at least about 60% Sp, at least about 65% Sp, at least about 70% Sp, at least about 75% Sp, at least about 80% Sp, at least about 85% Sp, at least about 90% Sp, or at least about 95% Sp, with the remainder Rp, or about 100% Sp. In some embodiments, an ASO used in the methods of the invention, including, but not limited to, any of the ASOs set forth herein in Table 1, comprises about 10% to about 100% Sp, about 15% to about 100% Sp, about 20% to about 100% Sp, about 25% to about 100% Sp, about 30% to about 100% Sp, about 35% to about 100% Sp, about 40% to about 100% Sp, about 45% to about 100% Sp, about 50% to about 100% Sp, about 55% to about 100% Sp, about 60% to about 100% Sp, about 65% to about 100% Sp, about 70% to about 100% Sp, about 75% to about 100% Sp, about 80% to about 100% Sp, about 85% to about 100% Sp, about 90% to about 100% Sp, or about 95% to about 100% Sp, about 20% to about 80% Sp, about 25% to about 75% Sp, about 30% to about 70% Sp, about 40% to about 60% Sp, or about 45% to about 55% Sp, with the remainder Rp.

Any of the ASOs described herein may contain a sugar moiety that comprises ribose or deoxyribose, as present in naturally occurring nucleotides, or a modified sugar moiety or sugar analog, including a morpholine ring. Non-limiting examples of modified sugar moieties include 2′ substitutions such as 2′-O-methyl (2′-O-Me), 2′-O-methoxyethyl (2′MOE), 2′-O-aminoethyl, 2′F; N3′->P5′ phosphoramidate, 2′dimethylaminooxyethoxy, 2′dimethylaminoethoxyethoxy, 2′-guanidinidium, 2′-O-guanidinium ethyl, carbamate modified sugars, and bicyclic modified sugars. In some embodiments, the sugar moiety modification is selected from 2′-O-Me, 2′F, and 2′MOE. In some embodiments, the sugar moiety modification is an extra bridge bond, such as in a locked nucleic acid (LNA). In some embodiments the sugar analog contains a morpholine ring, such as phosphorodiamidate morpholino (PMO). In some embodiments, the sugar moiety comprises a ribofuransyl or 2′deoxyribofuransyl modification. In some embodiments, the sugar moiety comprises 2′4′-constrained 2′O-methyloxyethyl (cMOE) modifications. In some embodiments, the sugar moiety comprises cEt 2′, 4′ constrained 2′-0 ethyl BNA modifications. In some embodiments, the sugar moiety comprises tricycloDNA (tcDNA) modifications. In some embodiments, the sugar moiety comprises ethylene nucleic acid (ENA) modifications. In some embodiments, the sugar moiety comprises MCE modifications. Modifications are known in the art and described in the literature, e.g., by Jarver, et al., 2014, “A Chemical View of Oligonucleotides for Exon Skipping and Related Drug Applications,” Nucleic Acid Therapeutics 24(1): 37-47, incorporated by reference for this purpose herein.

In some embodiments, each monomer of the ASO is modified in the same way, for example each linkage of the backbone of the ASO comprises a phosphorothioate linkage or each ribose sugar moiety comprises a 2′O-methyl modification. Such modifications that are present on each of the monomer components of an ASO are referred to as “uniform modifications.” In some embodiments, a combination of different modifications may be desired, for example, an ASO may comprise a combination of phosphorodiamidate linkages and sugar moieties comprising morpholine rings (morpholinos). Combinations of different modifications to an ASO are referred to as “mixed modifications” or “mixed chemistries.”

In some embodiments, the ASO comprises one or more backbone modification. In some embodiments, the ASO comprises one or more sugar moiety modification. In some embodiments, the ASO comprises one or more backbone modification and one or more sugar moiety modification. In some embodiments, the ASO comprises 2′MOE modifications and a phosphorothioate backbone. In some embodiments, the ASO comprises a phosphorodiamidate morpholino (PMO). In some embodiments, the ASO comprises a peptide nucleic acid (PNA). Any of the ASOs or any component of an ASO (e.g., a nucleobase, sugar moiety, backbone) described herein may be modified in order to achieve desired properties or activities of the ASO or reduce undesired properties or activities of the ASO. In some embodiments, an ASO or one or more component of any ASO may be modified to enhance binding affinity to a target sequence on a pre-mRNA transcript; reduce binding to any non-target sequence; reduce degradation by cellular nucleases (i.e., RNase H); improve uptake of the ASO into a cell and/or into the nucleus of a cell; alter the pharmacokinetics or pharmacodynamics of the ASO; and modulate the half-life of the ASO.

In some embodiments, the ASOs are comprised of 2′-O-(2-methoxyethyl) (MOE) phosphorothioate-modified nucleotides. ASOs comprised of such nucleotides are especially well-suited to the methods disclosed herein; oligomers having such modifications have been shown to have significantly enhanced resistance to nuclease degradation and increased bioavailability, making them suitable, for example, for oral delivery in some embodiments described herein. See e.g., Geary, et al., J Pharmacol Exp Ther. 2001; 296(3):890-7; Geary, et al., J Pharmacol Exp Ther. 2001; 296(3):898-904.

Methods of synthesizing ASOs will be known to one of skill in the art. Alternatively or in addition, ASOs may be obtained from a commercial source.

Unless specified otherwise, the left-hand end of single-stranded nucleic acid (e.g., pre-mRNA transcript, oligonucleotide, ASO, etc.) sequences is the 5′ end and the left-hand direction of single or double-stranded nucleic acid sequences is referred to as the 5′ direction. Similarly, the right-hand end or direction of a nucleic acid sequence (single or double stranded) is the 3′ end or direction. Generally, a region or sequence that is 5′ to a reference point in a nucleic acid is referred to as “upstream,” and a region or sequence that is 3′ to a reference point in a nucleic acid is referred to as “downstream.” Generally, the 5′ direction or end of an mRNA is where the initiation or start codon is located, while the 3′ end or direction is where the termination codon is located. In some embodiments, nucleotides that are upstream of a reference point in a nucleic acid may be designated by a negative number, while nucleotides that are downstream of a reference point may be designated by a positive number. For example, a reference point (e.g., an exon-exon junction in mRNA) may be designated as the “zero” site, and a nucleotide that is directly adjacent and upstream of the reference point is designated “minus one,” e.g., “−1,” while a nucleotide that is directly adjacent and downstream of the reference point is designated “plus one,” e.g., “+1.”

In some embodiments, the ASOs are complementary to (and bind to) a targeted portion of a SYNGAP1 pre-mRNA that is downstream (in the 3′ direction) of the 5′ splice site of the NSAE in a SYNGAP1 pre-mRNA (e.g., the direction designated by positive numbers relative to the 5′ splice site). In some embodiments, the ASOs are complementary to a targeted portion of the SYNGAP1 pre-mRNA that is within the region about +6 to about +500 relative to the 5′ splice site of the NSAE. In some embodiments, the ASO is not complementary to nucleotides +1 to +5 relative to the 5′ splice site (the first five nucleotides located downstream of the 5′ splice site). In some embodiments, the ASOs may be complementary to a targeted portion of a SYNGAP1 pre-mRNA that is within the region between nucleotides +6 and +100 relative to the 5′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 5′ splice site of the NSAE.

In some embodiments, the ASOs are complementary to (and bind to) a targeted portion of a target pre-mRNA that is downstream (in the 3′ direction) of the 5′ splice site of the NSAE in a target pre-mRNA (e.g., the direction designated by positive numbers relative to the 5′ splice site), wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the ASOs are complementary to a targeted portion of the target pre-mRNA that is within the region about +6 to about +500 relative to the 5′ splice site of the NSAE. In some embodiments, the ASO is not complementary to nucleotides +1 to +5 relative to the 5′ splice site (the first five nucleotides located downstream of the 5′ splice site). In some embodiments, the ASOs may be complementary to a targeted portion of a target pre-mRNA that is within the region between nucleotides +6 and +100 relative to the 5′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 5′ splice site of the NSAE.

In some embodiments, the ASOs are complementary to (and bind to) a targeted portion of a SYNGAP1 pre-mRNA that is upstream (in the 5′ direction) of the 3′ splice site of the NSAE in a SYNGAP1 pre-mRNA (e.g., the direction designated by negative numbers relative to the 3′ splice site). In some embodiments, the ASOs are complementary to a targeted portion of the SYNGAP1 pre-mRNA that is within the region about −16 to about −500 relative to the 3′ splice site of the NSAE. In some embodiments, the ASO is not complementary to nucleotides −1 to −15 relative to the 5′ splice site (the first five nucleotides located downstream of the 5′ splice site). In some embodiments, the ASOs may be complementary to a targeted portion of a SYNGAP1 pre-mRNA that is within the region between nucleotides −16 and −100 relative to the 3′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 3′ splice site of the NSAE.

In some embodiments, the ASOs are complementary to (and bind to) a targeted portion of a target pre-mRNA that is upstream (in the 5′ direction) of the 3′ splice site of the NSAE in a target pre-mRNA (e.g., the direction designated by negative numbers relative to the 3′ splice site), wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the ASOs are complementary to a targeted portion of the target pre-mRNA that is within the region about −16 to about −500 relative to the 3′ splice site of the NSAE. In some embodiments, the ASO is not complementary to nucleotides −1 to −15 relative to the 5′ splice site (the first five nucleotides located downstream of the 5′ splice site). In some embodiments, the ASOs may be complementary to a targeted portion of a target pre-mRNA that is within the region between nucleotides −16 and −100 relative to the 3′ splice site of the NSAE. In some aspects, the ASOs are complementary to a targeted portion that is within the region about +6 to about +500, about +6 to about +490, about +6 to about +480, about +6 to about +470, about +6 to about +460, about +6 to about +450, about +6 to about +440, about +6 to about +430, about +6 to about +420, about +6 to about +410, about +6 to about +400, about +6 to about +390, about +6 to about +380, about +6 to about +370, about +6 to about +360, about +6 to about +350, about +6 to about +340, about +6 to about +330, about +6 to about +320, about +6 to about +310, about +6 to about +300, about +6 to about +290, about +6 to about +280, about +6 to about +270, about +6 to about +260, about +6 to about +250, about +6 to about +240, about +6 to about +230, about +6 to about +220, about +6 to about +210, about +6 to about +200, about +6 to about +190, about +6 to about +180, about +6 to about +170, about +6 to about +160, about +6 to about +150, about +6 to about +140, about +6 to about +130, about +6 to about +120, about +6 to about +110, about +6 to about +100, about +6 to about +90, about +6 to about +80, about +6 to about +70, about +6 to about +60, about +6 to about +50, about +6 to about +40, about +6 to about +30, or about +6 to about +20 relative to 3′ splice site of the NSAE.

In some embodiments, the targeted portion of the SYNGAP1 pre-mRNA is within the region −4e relative to the 3′ splice site (5′ end) of the NSAE to +2e relative to the 5′ splice site (3′ end) of the NSAE.

In some embodiments, the targeted portion of the target pre-mRNA is within the region −4e relative to the 3′ splice site (5′ end) of the NSAE to +2e relative to the 5′ splice site (3′ end) of the NSAE, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. The ASOs may be of any length suitable for specific binding and effective enhancement of splicing. In some embodiments, the ASOs consist of 8 to 50 nucleobases. For example, the ASO may be 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, or 50 nucleobases in length. In some embodiments, the ASOs consist of more than 50 nucleobases. In some embodiments, the ASO is from 8 to 50 nucleobases, 8 to 40 nucleobases, 8 to 35 nucleobases, 8 to 30 nucleobases, 8 to 25 nucleobases, 8 to 20 nucleobases, 8 to 15 nucleobases, 9 to 50 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 50 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 50 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 50 nucleobases, 12 to 40 nucleobases, 12 to 35 nucleobases, 12 to 30 nucleobases, 12 to 25 nucleobases, 12 to 20 nucleobases, 12 to 15 nucleobases, 13 to 50 nucleobases, 13 to 40 nucleobases, 13 to 35 nucleobases, 13 to 30 nucleobases, 13 to 25 nucleobases, 13 to 20 nucleobases, 14 to 50 nucleobases, 14 to 40 nucleobases, 14 to 35 nucleobases, 14 to 30 nucleobases, 14 to 25 nucleobases, 14 to 20 nucleobases, 15 to 50 nucleobases, 15 to 40 nucleobases, 15 to 35 nucleobases, 15 to 30 nucleobases, 15 to 25 nucleobases, 15 to 20 nucleobases, 20 to 50 nucleobases, 20 to 40 nucleobases, 20 to 35 nucleobases, 20 to 30 nucleobases, 20 to 25 nucleobases, 25 to 50 nucleobases, 25 to 40 nucleobases, 25 to 35 nucleobases, or 25 to 30 nucleobases in length. In some embodiments, the ASOs are 15 nucleotides in length. In some embodiments, the ASOs are 16 nucleotides in length. In some embodiments, the ASOs are 17 nucleotides in length. In some embodiments, the ASOs are 18 nucleotides in length. In some embodiments, the ASOs are 25 nucleotides in length.

In some embodiments, two or more ASOs with different chemistries but complementary to the same targeted portion of the NSAE pre-mRNA are used. In some embodiments, two or more ASOs that are complementary to different targeted portions of the NSAE pre-mRNA are used.

In some embodiments, the antisense oligonucleotides of the invention are chemically linked to one or more moieties or conjugates, e.g., a targeting moiety or other conjugate that enhances the activity or cellular uptake of the oligonucleotide. Such moieties include, but are not limited to, a lipid moiety, e.g., as a cholesterol moiety, a cholesteryl moiety, an aliphatic chain, e.g., dodecandiol or undecyl residues, a polyamine or a polyethylene glycol chain, or adamantane acetic acid. Oligonucleotides comprising lipophilic moieties and preparation methods have been described in the published literature. In embodiments, the antisense oligonucleotide is conjugated with a moiety including, but not limited to, an abasic nucleotide, a polyether, a polyamine, a polyamide, a peptides, a carbohydrate, e.g., N-acetylgalactosamine (GalNAc), N—Ac-Glucosamine (GluNAc), or mannose (e.g., mannose-6-phosphate), a lipid, or a polyhydrocarbon compound. Conjugates can be linked to one or more of any nucleotides comprising the antisense oligonucleotide at any of several positions on the sugar, base or phosphate group, as understood in the art and described in the literature, e.g., using a linker. Linkers can include a bivalent or trivalent branched linker. In embodiments, the conjugate is attached to the 3′ end of the antisense oligonucleotide. Methods of preparing oligonucleotide conjugates are described, e.g., in U.S. Pat. No. 8,450,467, “Carbohydrate conjugates as delivery agents for oligonucleotides,” incorporated by reference herein.

In some embodiments, the nucleic acid to be targeted by an ASO is a SYNGAP1 pre-mRNA expressed in a cell, such as a eukaryotic cell. In some embodiments, the nucleic acid to be targeted by an ASO is a pre-mRNA expressed in a cell, such as a eukaryotic cell, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, the term “cell” may refer to a population of cells. In some embodiments, the cell is in a subject. In some embodiments, the cell is isolated from a subject. In some embodiments, the cell is ex vivo. In some embodiments, the cell is a condition or disease-relevant cell or a cell line. In some embodiments, the cell is in vitro (e.g., in cell culture).

### Pharmaceutical Compositions

Pharmaceutical compositions or formulations comprising the agent, e.g., antisense oligonucleotide, of the described compositions and for use in any of the described methods can be prepared according to conventional techniques well known in the pharmaceutical industry and described in the published literature. In embodiments, a pharmaceutical composition or formulation for treating a subject comprises an effective amount of any antisense oligomer as described herein, or a pharmaceutically acceptable salt, solvate, hydrate or ester thereof. The pharmaceutical formulation comprising an antisense oligomer may further comprise a pharmaceutically acceptable excipient, diluent or carrier.

Pharmaceutically acceptable salts are suitable for use in contact with the tissues of humans and lower animals without undue toxicity, irritation, allergic response, etc., and are commensurate with a reasonable benefit/risk ratio. (See, e.g., S. M. Berge, et al., J. Pharmaceutical Sciences, 66: 1-19 (1977), incorporated herein by reference for this purpose. The salts can be prepared in situ during the final isolation and purification of the compounds, or separately by reaction between the free base and a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other documented methodologies such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

In some embodiments, the compositions are formulated into any of many possible dosage forms such as, but not limited to, tablets, capsules, gel capsules, liquid syrups, soft gels, suppositories, and enemas. In some embodiments, the compositions are formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers. In some embodiments, a pharmaceutical formulation or composition of the present invention includes, but is not limited to, a solution, emulsion, microemulsion, foam or liposome-containing formulation (e.g., cationic or noncationic liposomes).

The pharmaceutical composition or formulation of the present invention may comprise one or more penetration enhancer, carrier, excipients or other active or inactive ingredients as appropriate and well known to those of skill in the art or described in the published literature. In some embodiments, liposomes also include sterically stabilized liposomes, e.g., liposomes comprising one or more specialized lipids. These specialized lipids result in liposomes with enhanced circulation lifetimes. In some embodiments, a sterically stabilized liposome comprises one or more glycolipids or is derivatized with one or more hydrophilic polymers, such as a polyethylene glycol (PEG) moiety. In some embodiments, a surfactant is included in the pharmaceutical formulation or compositions. The use of surfactants in drug products, formulations and emulsions is well known in the art. In some embodiments, the present invention employs a penetration enhancer to effect the efficient delivery of the antisense oligonucleotide, e.g., to aid diffusion across cell membranes and/or enhance the permeability of a lipophilic drug. In some embodiments, the penetration enhancers are a surfactant, fatty acid, bile salt, chelating agent, or non-chelating nonsurfactant.

In some embodiments, the pharmaceutical formulation comprises multiple antisense oligonucleotides. In some embodiments, the antisense oligonucleotide is administered in combination with another drug or therapeutic agent.

### Treatment of Subjects

Any of the compositions provided herein may be administered to an individual. “Individual” may be used interchangeably with “subject” or “patient.” An individual may be a mammal, for example a human or animal such as a non-human primate, a rodent, a rabbit, a rat, a mouse, a horse, a donkey, a goat, a cat, a dog, a cow, a pig, or a sheep. In some embodiments, the individual is a human. In some embodiments, the individual is a fetus, an embryo, or a child. In other embodiments, the individual may be another eukaryotic organism, such as a plant. In some embodiments, the compositions provided herein are administered to a cell ex vivo.

In some embodiments, the compositions provided herein are administered to an individual as a method of treating a disease or disorder. In some embodiments, the individual has a genetic disease, such as any of the diseases described herein. In some embodiments, the individual is at risk of having the disease, such as any of the diseases described herein. In some embodiments, the individual is at increased risk of having a disease or disorder caused by insufficient amount of a protein or insufficient activity of a protein. In some embodiments, if an individual is “at an increased risk” of having a disease or disorder caused by insufficient amount of a protein or insufficient activity of a protein, the method involves preventative or prophylactic treatment. For example, an individual may be at an increased risk of having such a disease or disorder because of family history of the disease. Typically, individuals at an increased risk of having such a disease or disorder benefit from prophylactic treatment (e.g., by preventing or delaying the onset or progression of the disease or disorder). In some embodiments, a fetus is treated in utero, e.g., by administering the ASO composition to the fetus directly or indirectly (e.g., via the mother).

Suitable routes for administration of ASOs of the present invention may vary depending on cell type to which delivery of the ASOs is desired. Multiple tissues and organs can be affected by autosomal dominant mental retardation. In some embodiments, the liver can be the most significantly affected tissue. The ASOs of the present invention may be administered to patients parenterally, for example, by intrathecal injection, intracerebroventricular injection, intraperitoneal injection, intramuscular injection, subcutaneous injection, or intravenous injection.

Methods of Identifying Additional ASOs that Prevent Alternative Splicing

Also within the scope of the present disclosure are methods for identifying or determining ASOs that prevent alternative splicing of a SYNGAP1 pre-mRNA. For example, a method can comprise identifying or determining ASOs that prevent alternative splicing of a SYNGAP1 pre-mRNA. Also within the scope of the present disclosure are methods for identifying or determining ASOs that prevent alternative splicing of a target pre-mRNA, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. For example, a method can comprise identifying or determining ASOs that prevent alternative splicing of a target pre-mRNA. ASOs that specifically hybridize to different nucleotides within the target region of the pre-mRNA may be screened to identify or determine ASOs that improve the extent of canonical splicing. In some embodiments, the ASO may block or interfere with the binding site(s) of a splicing silencer. Any method known in the art may be used to identify (determine) an ASO that when hybridized to the target region of the NSAE results in the desired effect (e.g., exon inclusion, protein or functional RNA production). These methods also can be used for identifying ASOs that prevent alternative splicing of the NSAE by binding to a targeted region in an intron flanking the NSAE. An example of a method that may be used is provided below.

A round of screening, referred to as an ASO “walk” may be performed using ASOs that have been designed to hybridize to a target region of a pre-mRNA. For example, the ASOs used in the ASO walk can be tiled every 5 nucleotides from approximately 100 nucleotides upstream of the 3′ splice site of the NSAE (e.g., a portion of sequence of the intron located upstream of the target/NSAE) to approximately 100 nucleotides downstream of the 3′ splice site of the target/NSAE and/or from approximately 100 nucleotides upstream of the 5′ splice site of the NSAE to approximately 100 nucleotides downstream of the 5′ splice site of the target/NSAE (e.g., a portion of sequence of the intron located downstream of the target/NSAE). For example, a first ASO of 18 nucleotides in length may be designed to specifically hybridize to nucleotides +6 to +23 relative to the 5′ splice site of the target/NSAE. A second ASO is designed to specifically hybridize to nucleotides +11 to +28 relative to the 5′ splice site of the target/NSAE. ASOs are designed as such spanning the target region of the pre-mRNA. In some embodiments, the ASOs can be tiled more closely, e.g., every 1, 2, 3, or 4 nucleotides. Further, the ASOs can be tiled from 100 nucleotides downstream of the 5′ splice site, to 100 nucleotides upstream of the 3′ splice site. In some embodiments, the ASOs can be tiled from about 572 nucleotides upstream of the 3′ splice site, to about 500 nucleotides downstream of the 5′ splice site. In some embodiments, the ASOs can be tiled from about 500 nucleotides upstream of the 3′ splice site, to about 572 nucleotides downstream of the 3′ splice site.

One or more ASOs, or a control ASO (an ASO with a scrambled sequence, sequence that is not expected to hybridize to the target region) are delivered, for example by transfection, into a disease-relevant cell line that expresses the target pre-mRNA (e.g., a NSAE pre-mRNA described herein). The canonical splicing promoting effects of each of the ASOs may be assessed by any method known in the art, for example by reverse transcriptase (RT)-PCR using primers, such as primers spanning the region containing the NSAE or canonical exon. A decrease or absence of a longer or shorter RT-PCR product than a canonical exon produced using primers spanning the region containing the NSAE or canonical exon in ASO-treated cells as compared to in control ASO-treated cells indicates that exclusion of the NSAE has been increased. In some embodiments, the canonical splicing efficiency or the ratio of canonically spliced mRNA to NSAE containing mRNA may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

A second round of screening, referred to as an ASO “micro-walk” may be performed using ASOs that have been designed to hybridize to a target region of a pre-mRNA. The ASOs used in the ASO micro-walk are tiled every 1 nucleotide to further refine the nucleotide acid sequence of the pre-mRNA that when hybridized with an ASO results in modulation of NSAE exclusion.

Regions defined by ASOs that promote inclusion target NSAE are explored in greater detail by means of an ASO “micro-walk”, involving ASOs spaced in 1-nt steps, as well as longer ASOs, typically 15-25 nt.

As described for the ASO walk above, the ASO micro-walk is performed by delivering one or more ASOs, or a control ASO (an ASO with a scrambled sequence, sequence that is not expected to hybridize to the target region), for example by transfection, into a disease-relevant cell line that expresses the target pre-mRNA. The splicing-inducing effects of each of the ASOs may be assessed by any method known in the art, for example by reverse transcriptase (RT)-PCR using primers that span the NSAE, as described herein. A decrease or absence of a longer or shorter RT-PCR product than a canonical exon produced using primers spanning the region containing the NSAE or canonical exon in ASO-treated cells as compared to in control ASO-treated cells indicates that exclusion of the NSAE has been increased. In some embodiments, the canonical splicing efficiency or the ratio of canonically spliced mRNA to retained NSAE containing mRNA may be improved using the ASOs described herein. The amount of protein or functional RNA that is encoded by the target pre-mRNA can also be assessed to determine whether each ASO achieved the desired effect (e.g., enhanced functional protein production). Any method known in the art for assessing and/or quantifying protein production, such as Western blotting, flow cytometry, immunofluorescence microscopy, and ELISA, can be used.

ASOs that when hybridized to a region of a pre-mRNA result in canonical splicing and increased protein production may be tested in vivo using animal models, for example transgenic mouse models in which the full-length human gene has been knocked-in or in humanized mouse models of disease. Suitable routes for administration of ASOs may vary depending on the disease and/or the cell types to which delivery of the ASOs is desired. ASOs may be administered, for example, by intrathecal injection, intracerebroventricular injection, intraperitoneal injection, intramuscular injection, subcutaneous injection, or intravenous injection. Following administration, the cells, tissues, and/or organs of the model animals may be assessed to determine the effect of the ASO treatment by for example evaluating splicing (efficiency, rate, extent) and protein production by methods known in the art and described herein. The animal models may also be any phenotypic or behavioral indication of the disease or disease severity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In some embodiments, the exemplary NMD-inducing Alternative 5′ splice site or 3′ splice site targets comprise the genes listed in Table 5A, Table 5B or Table 5C. In some embodiments, the compositions as provided herein comprise a NSAE-modulating agent that modulates expression of a target protein listed in Table 5A, Table 5B or Table 5C. In some embodiments, the method as provided herein are used to modulate expression of a target protein listed in Table 5A, Table 5B or Table 5C by administering a NSAE-modulating agent.

For example, in some embodiments, GRN (Gene ID: 2896) is an exemplary target of the NMD-inducing Alternative 3′ splice site event. In some embodiments, the compositions or the methods as described herein may be used to treat GRN-related frontotemporal dementia (OMIM (Online Mendelian Inheritance in Man)): 607485) related to the haploinsufficiency of GRN in the central nervous system (CNS).

For another example, in some embodiments, SHANK3 (Gene ID: 85358) is an exemplary target of the NMD-inducing Alternative 3′ splice site event. In some embodiments, the compositions or the methods as described herein may be used to treat Phelan-McDermid syndrome; Autism spectrum disorder (OMIM: 606232) related to the haploinsufficiency of SHANK3 in the CNS.

For another example, in some embodiments, PRPF3 (Gene ID: 9129) is an exemplary target of the NMD-inducing Alternative 3′ splice site event. In some embodiments, the compositions or the methods as described herein may be used to treat Retinitis pigmentosa 18 (OMIM: 601414) related to the haploinsufficiency of PRPF3 in the eye.

For another example, in some embodiments, PRPF4 (Gene ID: 9128) is an exemplary target of the NMD-inducing Alternative 5′ splice site event or the NMD-inducing Alternative 3′ splice site event. In some embodiments, the compositions or the methods as described herein may be used to treat Retinitis pigmentosa 70 (OMIM: 615922) related to the haploinsufficiency of PRPF4 in the eye.

For another example, in some embodiments, SYNGAP1 (Gene ID: 8831) is an exemplary target of the NMD-inducing Alternative 5′ splice site event or the NMD-inducing Alternative 3′ splice site event. In some embodiments, the compositions or the methods as described herein may be used to treat AD mental retardation 5 (OMIM: 612621) related to the haploinsufficiency of SYNGAP1 in the CNS.

For another example, in some embodiments, ABCA4 (Gene ID: 24) is an exemplary target of the NMD-inducing Alternative 3′ splice site event. In some embodiments, the compositions or the methods as described herein may be used to treat Stargardt disease (OMIM: 248200) related to the autosomal recessive ABCA4 in the eye.

For another example, in some embodiments, DNAJB2 (Gene ID: 3300) is an exemplary target of the NMD-inducing Alternative 5′ splice site event or the NMD-inducing Alternative 3′ splice site event. In some embodiments, the compositions or the methods as described herein may be used to modulate the pathway related to defective DNAJB2 in the eye.

For another example, in some embodiments, CLPX (Gene ID: 10845) is an exemplary target of the NMD-inducing Alternative 5′ splice site event. In some embodiments, the compositions or the methods as described herein may be used to modulate the pathway related to defective CLPX in the eye.

For another example, in some embodiments, HSPA9 (Gene ID: 3313) is an exemplary target of the NMD-inducing Alternative 3′ splice site event. In some embodiments, the compositions or the methods as described herein may be used to modulate the pathway related to defective HSPA9 in the eye.

For another example, in some embodiments, SLC4A11 (Gene ID: 83959) is an exemplary target of the NMD-inducing Alternative 3′ splice site event. In some embodiments, the compositions or the methods as described herein may be used to treat Fuch's corneal dystrophy (OMIM: 217700) related to the autosomal recessive SLC4A11 in the eye.

For another example, in some embodiments, OPTN (Gene ID: 10133) is an exemplary target of the NMD-inducing Alternative 3′ splice site event. In some embodiments, the compositions or the methods as described herein may be used to modulate the pathway related to defective OPTN in the eye or CNS.

For another example, in some embodiments, eIF2B5 (Gene ID: 8893) is an exemplary target of the NMD-inducing Alternative 5′ splice site event. In some embodiments, the compositions or the methods as described herein may be used to modulate the pathway related to defective eIF2B5 in the eye.

For another example, in some embodiments, SIRT3 (Gene ID: 23410) is an exemplary target of the NMD-inducing Alternative 3′ splice site event. In some embodiments, the compositions or the methods as described herein may be used to modulate the pathway related to defective SIRT3 in the eye.

For another example, in some embodiments, STAT3 (Gene ID: 6774) is an exemplary target of the NMD-inducing Alternative 5′ splice site event. In some embodiments, the compositions or the methods as described herein may be used to modulate the pathway related to defective STAT3 in the eye.

For another example, in some embodiments, KCNQ2 (Gene ID: 3785) is an exemplary target of the NMD-inducing Alternative 5′ splice site event or the NMD-inducing Alternative 3′ splice site event. In some embodiments, the compositions or the methods as described herein may be used to treat early-onset epileptic encephalopathy (OMIM: 613720) related to the haploinsufficiency of KCNQ2 in the CNS.

For another example, in some embodiments, CACNA1G (Gene ID: 8913) is an exemplary target of the NMD-inducing Alternative 3′ splice site event. In some embodiments, the compositions or the methods as described herein may be used to treat Spinocerebellar ataxia 42 [AD] (OMIM: 616795) related to the potential haploinsufficiency of CACNA1G in the CNS.

For another example, in some embodiments, GBA (Gene ID: 2629) is an exemplary target of the NMD-inducing Alternative 5′ splice site event or the NMD-inducing Alternative 3′ splice site event. In some embodiments, the compositions or the methods as described herein may be used to treat Parkinson disease (OMIM: 168600) related to the haploinsufficiency of GBA in the CNS.

For another example, in some embodiments, KL (Gene ID: 9365) is an exemplary target of the NMD-inducing Alternative 5′ splice site event. In some embodiments, the compositions or the methods as described herein may be used to modulate the pathway related to defective KL in the CNS.

For another example, in some embodiments, AKT2 (Gene ID: 208) is an exemplary target of the NMD-inducing Alternative 3′ splice site event. In some embodiments, the compositions or the methods as described herein may be used to modulate the pathway related to defective AKT2 in the eye.

For another example, in some embodiments, EIF2AK3 (Gene ID: 9451) is an exemplary target of the NMD-inducing Alternative 3′ splice site event. In some embodiments, the compositions or the methods as described herein may be used to modulate the pathway related to defective EIF2AK3 in the eye.

For another example, in some embodiments, SEMA4A (Gene ID: 64218) is an exemplary target of the NMD-inducing Alternative 3′ splice site event. In some embodiments, the compositions or the methods as described herein may be used to modulate the pathway related to defective SEMA4A in the eye.

For another example, in some embodiments, SEMA3B (Gene ID: 10509) is an exemplary target of the NMD-inducing Alternative 3′ splice site event. In some embodiments, the compositions or the methods as described herein may be used to modulate the pathway related to defective SEMA3B in the eye.

For another example, in some embodiments, SEMA3F (Gene ID: 10505) is an exemplary target of the NMD-inducing Alternative 5′ splice site event or the NMD-inducing Alternative 3′ splice site event. In some embodiments, the compositions or the methods as described herein may be used to modulate the pathway related to defective SEMA3F in the eye.

For another example, in some embodiments, SLC4A11 (Gene ID: 83959) is an exemplary target of the NMD-inducing Alternative 3′ splice site event. In some embodiments, the compositions or the methods as described herein may be used to treat Corneal dystrophy, Fuchs endothelial, 4 (OMIM: 613268) related to the potential haploinsufficiency of SLC4A11 in the eye.

For another example, in some embodiments, JAG1 (Gene ID: 182) is an exemplary target of the NMD-inducing Alternative 5′ splice site event. In some embodiments, the compositions or the methods as described herein may be used to treat Alagille syndrome (OMIM: 118450) related to the haploinsufficiency of JAG1 in the liver.

For another example, in some embodiments, CHD2 (Gene ID: 1106) is an exemplary target of the NMD-inducing Alternative 3′ splice site event. In some embodiments, the compositions or the methods as described herein may be used to treat Epileptic encephalopathy, childhood-onset (OMIM: 615369) related to the haploinsufficiency of CHD2 in the CNS.

For another example, in some embodiments, MCEE (Gene ID: 84693) is an exemplary target of the NMD-inducing Alternative 5′ splice site event. In some embodiments, the compositions or the methods as described herein may be used to treat Methylmalonyl-CoA epimerase deficiency (OMIM: 251120) related to the autosomal recessive MCEE in the liver.

For another example, in some embodiments, TRAPPC6B (Gene ID: 122553) is an exemplary target of the NMD-inducing Alternative 5′ splice site event. In some embodiments, the compositions or the methods as described herein may be used to treat Neurodevelopmental disorder with microcephaly, epilepsy, and brain atrophy (OMIM: 617862) related to the autosomal recessive TRAPPC6B in the CNS.

For another example, in some embodiments, AP3B2 (Gene ID: 8120) is an exemplary target of the NMD-inducing Alternative 3′ splice site event. In some embodiments, the compositions or the methods as described herein may be used to treat Epileptic encephalopathy, early infantile, 48 (OMIM: 617276) related to the autosomal recessive AP3B2 in the CNS.

For another example, in some embodiments, TTC19 (Gene ID: 54902) is an exemplary target of the NMD-inducing Alternative 5′ splice site event. In some embodiments, the compositions or the methods as described herein may be used to treat Mitochondrial complex III deficiency, nuclear type 2 (OMIM: 615157) related to the autosomal recessive TTC19 in the CNS.

For another example, in some embodiments, DMTF1 (Gene ID: 9988) is an exemplary target of the NMD-inducing Alternative 3′ splice site event. In some embodiments, the compositions or the methods as described herein may be used to modulate the tumor suppressor activity of DMTF1. In some embodiments, the compositions or the methods as described herein may be used to increase the tumor suppressor activity of DMTF1, thereby treating cancer.

For another example, in some embodiments, HDAC3 (Gene ID: 8841) is an exemplary target of the NMD-inducing Alternative 3′ splice site event. In some embodiments, the compositions or the methods as described herein may be used to modulate the tumor suppressor activity of HDAC3. In some embodiments, the compositions or the methods as described herein may be used to increase the tumor suppressor activity of HDAC3, thereby treating cancer.

For another example, in some embodiments, DAB2IP (Gene ID: 153090) is an exemplary target of the NMD-inducing Alternative 5′ splice site event. In some embodiments, the compositions or the methods as described herein may be used to modulate the tumor suppressor activity of DAB2IP. In some embodiments, the compositions or the methods as described herein may be used to increase the tumor suppressor activity of DAB2IP, thereby treating cancer.

For another example, in some embodiments, CD4 (Gene ID: 920) is an exemplary target of the NMD-inducing Alternative 5′ splice site event. In some embodiments, the compositions or the methods as described herein may be used to modulate the tumor suppressor activity of CD4. In some embodiments, the compositions or the methods as described herein may be used to increase the tumor suppressor activity of CD4, thereby treating cancer.

For another example, in some embodiments, TP53BP2 (Gene ID: 7159) is an exemplary target of the NMD-inducing Alternative 5′ splice site event. In some embodiments, the compositions or the methods as described herein may be used to modulate the tumor suppressor activity of TP53BP2. In some embodiments, the compositions or the methods as described herein may be used to increase the tumor suppressor activity of TP53BP2, thereby treating cancer.

For another example, in some embodiments, BAP1 (Gene ID: 8314) is an exemplary target of the NMD-inducing Alternative 3′ splice site event. In some embodiments, the compositions or the methods as described herein may be used to modulate the tumor suppressor activity of BAP1. In some embodiments, the compositions or the methods as described herein may be used to increase the tumor suppressor activity of BAP1, thereby treating cancer.

For another example, in some embodiments, CHFR (Gene ID: 55743) is an exemplary target of the NMD-inducing Alternative 3′ splice site event. In some embodiments, the compositions or the methods as described herein may be used to modulate the tumor suppressor activity of CHFR. In some embodiments, the compositions or the methods as described herein may be used to increase the tumor suppressor activity of CHFR, thereby treating cancer.

For another example, in some embodiments, IRF3 (Gene ID: 3661) is an exemplary target of the NMD-inducing Alternative 5′ splice site event. In some embodiments, the compositions or the methods as described herein may be used to modulate the tumor suppressor activity of IRF3. In some embodiments, the compositions or the methods as described herein may be used to increase the tumor suppressor activity of IRF3, thereby treating cancer.

For another example, in some embodiments, SIRT2 (Gene ID: 22933) is an exemplary target of the NMD-inducing Alternative 3′ splice site event. In some embodiments, the compositions or the methods as described herein may be used to modulate the tumor suppressor activity of SIRT2. In some embodiments, the compositions or the methods as described herein may be used to increase the tumor suppressor activity of SIRT2, thereby treating cancer.

For another example, in some embodiments, RBL1 (Gene ID: 5933) is an exemplary target of the NMD-inducing Alternative 5′ splice site event. In some embodiments, the compositions or the methods as described herein may be used to modulate the tumor suppressor activity of RBL1. In some embodiments, the compositions or the methods as described herein may be used to increase the tumor suppressor activity of RBL1, thereby treating cancer.

In some embodiments, the compositions or the methods as described herein are used to treat a condition or disease selected from the group consisting of dementia, GRN-related frontotemporal dementia, Phelan-McDermid syndrome; Autism spectrum disorder, Retinitis pigmentosa 18, Retinitis pigmentosa 70, autosomal dominant mental retardation-5 (MRD5; AD Mental Retardation 5), Stargardt disease, Fuch's corneal dystrophy, early-onset epileptic encephalopathy, Spinocerebellar ataxia 42 [AD], Parkinson disease, Corneal dystrophy, Fuchs endothelial 4, Alagille syndrome, Epileptic encephalopathy, childhood-onset Methylmalonyl-CoA epimerase deficiency, Neurodevelopmental disorder with microcephaly, epilepsy, and brain atrophy, Epileptic encephalopathy, early infantile, 48, Mitochondrial complex III deficiency, nuclear type 2, and tumor. In some embodiments, the compositions or the methods as described herein are used to prevent, delay the progression and/or development of a sign or a symptom, or alleviate a sign or a symptom associated with a condition or disease selected from the group consisting of dementia, GRN-related frontotemporal dementia, Phelan-McDermid syndrome; Autism spectrum disorder, Retinitis pigmentosa 18, Retinitis pigmentosa 70, autosomal dominant mental retardation-5 (MRD5; AD Mental Retardation 5), Stargardt disease, Fuch's corneal dystrophy, early-onset epileptic encephalopathy, Spinocerebellar ataxia 42 [AD], Parkinson disease, Corneal dystrophy, Fuchs endothelial 4, Alagille syndrome, Epileptic encephalopathy, childhood-onset Methylmalonyl-CoA epimerase deficiency, Neurodevelopmental disorder with microcephaly, epilepsy, and brain atrophy, Epileptic encephalopathy, early infantile, 48, Mitochondrial complex III deficiency, nuclear type 2, and tumor. In some embodiments, the compositions or the methods as described herein are used to treat a condition or disease of Table 5C. In some embodiments, the compositions or the methods as described herein are used to prevent, delay the progression and/or development of a sign or a symptom, or alleviate a sign or a symptom associated with a condition or disease of Table 5C.

TABLE 5A

| | Genomic | | | | SEQ | Event | |
|---|---|---|---|---|---|---|---|
| | sequence | Region coordinate | | | ID | coordinate | Event |
| Gene | SEQ ID NO: | chrm | start | end | NO: | exon | type* |
| ABCC3 | 1118 | chr17 | 50658497 | 50659181 | 15073 | chr17 | alt_3ss |
| | | | | | | 50659182 | |
| | | | | | | 50659236+ | |
| ABCC5 | 1119 | chr3 | 183972035 | 183977516 | 15074 | chr3 | alt_3ss |
| | | | | | | 183971920 | |
| | | | | | | 183972034− | |
| ABCC8 | 1120 | chr11 | 17407210 | 17407353 | 15075 | chr11 | alt_3ss |
| | | | | | | 17407130 | |
| | | | | | | 17407209− | |
| ACOX2 | 1125 | chr3 | 58534609 | 58534946 | 15076 | chr3 | alt_3ss |
| | | | | | | 58534523 | |
| | | | | | | 58534608− | |
| ACSF3 | 1126 | chr16 | 89133263 | 89145213 | 15077 | chr16 | alt_3ss |
| | | | | | | 89145214 | |
| | | | | | | 89145266+ | |
| ADAM17 | 1128 | chr2 | 9526265 | 9527785 | 15078 | chr2 | alt_3ss |
| | | | | | | 9526245 | |
| | | | | | | 9526264− | |
| ADAMTS13 | 1129 | chr9 | 133428772 | 133429943 | 15079 | chr9 | alt_3ss |
| | | | | | | 133429944 | |
| | | | | | | 133430031+ | |
| ALAD | 1131 | chr9 | 113392336 | 113393446 | 15080 | chr9 | alt_3ss |
| | | | | | | 113392170 | |
| | | | | | | 113392335− | |
| ANKS3 | 1134 | chr16 | 4726855 | 4726978 | 15081 | chr16 | alt_3ss |
| | | | | | | 4726781 | |
| | | | | | | 4726854− | |
| ANO4 | 1135 | chr12 | 100971407 | 100974782 | 15082 | chr12 | alt_3ss |
| | | | | | | 100974783 | |
| | | | | | | 100974844+ | |
| AP3B2 | 1136 | chr15 | 82663296 | 82663559 | 15083 | chr15 | alt_3ss |
| | | | | | | 82663234 | |
| | | | | | | 82663295− | |
| AP5Z1 | 1138 | chr7 | 4786429 | 4787595 | 15084 | chr7 | alt_3ss |
| | | | | | | 4787596 | |
| | | | | | | 4787633+ | |
| ARNTL | 1139 | chr11 | 13375763 | 13376544 | 15085 | chr11 | alt_3ss |
| | | | | | | 13376545 | |
| | | | | | | 13376639+ | |
| BBS2 | 1142 | chr16 | 56498694 | 56499777 | 15086 | chr16 | alt_3ss |
| | | | | | | 56498569 | |
| | | | | | | 56498693− | |
| BRD9 | 1144 | chr5 | 889767 | 891154 | 15087 | chr5 | alt_3ss |
| | | | | | | 889648 | |
| | | | | | | 889766− | |
| CALM3 | 1145 | chr19 | 46608328 | 46608481 | 15088 | chr19 | alt_3ss |
| | | | | | | 46608482 | |
| | | | | | | 46608923+ | |
| CARS1 | 1146 | chr11 | 3041032 | 3042164 | 15089 | chr11 | alt_3ss |
| | | | | | | 3040985 | |
| | | | | | | 3041031− | |
| CASP9 | 1147 | chr1 | 15507134 | 15507872 | 15090 | chr1 | alt_3ss |
| | | | | | | 15507076 | |
| | | | | | | 15507133− | |
| CD58 | 1152 | chr1 | 116519303 | 116521905 | 15091 | chr1 | alt_3ss |
| | | | | | | 116519268 | |
| | | | | | | 116519302− | |
| CHFR | 1154 | chr12 | 132848258 | 132848640 | 15092 | chr12 | alt_3ss |
| | | | | | | 132848156 | |
| | | | | | | 132848257− | |
| CLCN2 | 1155 | chr3 | 184354715 | 184354903 | 15093 | chr3 | alt_3ss |
| | | | | | | 184354608 | |
| | | | | | | 184354714− | |
| CLCN2 | 1155 | chr3 | 184354715 | 184354903 | 15094 | chr3 | alt_3ss |
| | | | | | | 184354659 | |
| | | | | | | 184354714− | |
| CNOT3 | 1156 | chr19 | 54144333 | 54145541 | 15095 | chr19 | alt_3ss |
| | | | | | | 54145542 | |
| | | | | | | 54145597+ | |
| CNTROB | 1157 | chr17 | 7935146 | 7936237 | 15096 | chr17 | alt_3ss |
| | | | | | | 7936238 | |
| | | | | | | 7936365+ | |
| CTF1 | 1161 | chr16 | 30896669 | 30899306 | 15097 | chr16 | alt_3ss |
| | | | | | | 30899307 | |
| | | | | | | 30899414+ | |
| CTF1 | 1161 | chr16 | 30896669 | 30899306 | 15098 | chr16 | alt_3ss |
| | | | | | | 30899307 | |
| | | | | | | 30899417+ | |
| CYP3A5 | 1163 | chr7 | 99666811 | 99666951 | 15099 | chr7 | alt_3ss |
| | | | | | | 99666690 | |
| | | | | | | 99666810− | |
| DHDDS | 1169 | chr1 | 26438285 | 26442707 | 15100 | chr1 | alt_3ss |
| | | | | | | 26442708 | |
| | | | | | | 26442730+ | |
| DNHD1 | 1171 | chr11 | 6565992 | 6566100 | 15101 | chr11 | alt_3ss |
| | | | | | | 6566101 | |
| | | | | | | 6566240+ | |
| ENTPD4 | 1177 | chr8 | 23434682 | 23435391 | 15102 | chr8 | alt_3ss |
| | | | | | | 23434479 | |
| | | | | | | 23434681− | |
| ESRRA | 1178 | chr11 | 64307505 | 64313853 | 15103 | chr11 | alt_3ss |
| | | | | | | 64313854 | |
| | | | | | | 64313950+ | |
| ESS2 | 1179 | chr22 | 19138480 | 19139158 | 15104 | chr22 | alt_3ss |
| | | | | | | 19138318 | |
| | | | | | | 19138479− | |
| ETAA1 | 1180 | chr2 | 67402975 | 67404996 | 15105 | chr2 | alt_3ss |
| | | | | | | 67404997 | |
| | | | | | | 67405235+ | |
| FASTKD3 | 1184 | chr5 | 7861774 | 7862822 | 15106 | chr5 | alt_3ss |
| | | | | | | 7861653 | |
| | | | | | | 7861773− | |
| FREM1 | 1186 | chr9 | 14776254 | 14784369 | 15107 | chr9 | alt_3ss |
| | | | | | | 14776204 | |
| | | | | | | 14776253− | |
| FREM1 | 1186 | chr9 | 14776257 | 14784369 | 15108 | chr9 | alt_3ss |
| | | | | | | 14776204 | |
| | | | | | | 14776256− | |
| GAS8 | 1187 | chr16 | 90027723 | 90031189 | 15109 | chr16 | alt_3ss |
| | | | | | | 90031190 | |
| | | | | | | 90031298+ | |
| GGA3 | 1190 | chr17 | 75243259 | 75243446 | 15110 | chr17 | alt_3ss |
| | | | | | | 75243167 | |
| | | | | | | 75243258− | |
| GGT1 | 1191 | chr22 | 24623917 | 24627409 | 15111 | chr22 | alt_3ss |
| | | | | | | 24627410 | |
| | | | | | | 24627431+ | |
| GLMN | 1192 | chr1 | 92290331 | 92291417 | 15112 | chr1 | alt_3ss |
| | | | | | | 92290307 | |
| | | | | | | 92290330− | |
| GLMN | 1192 | chr1 | 92266594 | 92266699 | 15113 | chr1 | alt_3ss |
| | | | | | | 92266493 | |
| | | | | | | 92266593− | |
| GRB14 | 1194 | chr2 | 164508604 | 164508741 | 15114 | chr2 | alt_3ss |
| | | | | | | 164508551 | |
| | | | | | | 164508603− | |
| HDAC3 | 1195 | chr5 | 141628213 | 141628558 | 15115 | chr5 | alt_3ss |
| | | | | | | 141628188 | |
| | | | | | | 141628212− | |
| HP1BP3 | 1197 | chr1 | 20749976 | 20757165 | 15116 | chr1 | alt_3ss |
| | | | | | | 20749883 | |
| | | | | | | 20749975− | |
| HPS4 | 1199 | chr22 | 26470923 | 26472301 | 15117 | chr22 | alt_3ss |
| | | | | | | 26470814 | |
| | | | | | | 26470922− | |
| IDUA | 1200 | chr4 | 1001564 | 1001650 | 15118 | chr4 | alt_3ss |
| | | | | | | 1001651 | |
| | | | | | | 1001678+ | |
| IFT122 | 1201 | chr3 | 129514555 | 129515399 | 15119 | chr3 | alt_3ss |
| | | | | | | 129515400 | |
| | | | | | | 129515487+ | |
| IMPDH1 | 1203 | chr7 | 128400205 | 128400332 | 15120 | chr7 | alt_3ss |
| | | | | | | 128400183 | |
| | | | | | | 128400204− | |
| KCTD3 | 1206 | chr1 | 215577729 | 215577928 | 15121 | chr1 | alt_3ss |
| | | | | | | 215577929 | |
| | | | | | | 215578000+ | |
| KYAT1 | 1208 | chr9 | 128836106 | 128836801 | 15122 | chr9 | alt_3ss |
| | | | | | | 128836074 | |
| | | | | | | 128836105− | |
| MAP3K7 | 1213 | chr6 | 90560235 | 90561621 | 15123 | chr6 | alt_3ss |
| | | | | | | 90560215 | |
| | | | | | | 90560234− | |
| NBEAL2 | 1219 | chr3 | 46999118 | 46999269 | 15124 | chr3 | alt_3ss |
| | | | | | | 46999270 | |
| | | | | | | 46999314+ | |
| NOP58 | 1223 | chr2 | 202290458 | 202291020 | 15125 | chr2 | alt_3ss |
| | | | | | | 202291021 | |
| | | | | | | 202291124+ | |
| NPHP1 | 1224 | chr2 | 110178936 | 110179623 | 15126 | chr2 | alt_3ss |
| | | | | | | 110178548 | |
| | | | | | | 110178935− | |
| NUP188 | 1226 | chr9 | 128987718 | 128987915 | 15127 | chr9 | alt_3ss |
| | | | | | | 128987916 | |
| | | | | | | 128988046+ | |
| PHKA2 | 1230 | chrX | 18936202 | 18938626 | 15128 | chrX | alt_3ss |
| | | | | | | 18936151 | |
| | | | | | | 18936201− | |
| PIDD1 | 1231 | chr11 | 803086 | 803173 | 15129 | chr11 | alt_3ss |
| | | | | | | 802892 | |
| | | | | | | 803085− | |
| PIDD1 | 1231 | chr11 | 803648 | 804093 | 15130 | chr11 | alt_3ss |
| | | | | | | 803588 | |
| | | | | | | 803647− | |
| PKD1 | 1232 | chr16 | 2105663 | 2105864 | 15131 | chr16 | alt_3ss |
| | | | | | | 2105475 | |
| | | | | | | 2105662− | |
| PLA2G6 | 1233 | chr22 | 38115850 | 38116074 | 15132 | chr22 | alt_3ss |
| | | | | | | 38115682 | |
| | | | | | | 38115849− | |
| PLD2 | 1234 | chr17 | 4810952 | 4814366 | 15133 | chr17 | alt_3ss |
| | | | | | | 4814367 | |
| | | | | | | 4814417+ | |
| PLD2 | 1234 | chr17 | 4815935 | 4816541 | 15134 | chr17 | alt_3ss |
| | | | | | | 4816542 | |
| | | | | | | 4816619+ | |
| PLEKHG5 | 1235 | chr1 | 6471241 | 6471487 | 15135 | chr1 | alt_3ss |
| | | | | | | 6471101 | |
| | | | | | | 6471240− | |
| PRMT7 | 1239 | chr16 | 68352410 | 68353394 | 15136 | chr16 | alt_3ss |
| | | | | | | 68353395 | |
| | | | | | | 68353491+ | |
| PRODH | 1240 | chr22 | 18919632 | 18919705 | 15137 | chr22 | alt_3ss |
| | | | | | | 18919598 | |
| | | | | | | 18919631− | |
| PRPF3 | 1241 | chr1 | 150344262 | 150344375 | 15138 | chr1 | alt_3ss |
| | | | | | | 150344376 | |
| | | | | | | 150344433+ | |
| PRPF4 | 1242 | chr9 | 113284390 | 113286192 | 15139 | chr9 | alt_3ss |
| | | | | | | 113286193 | |
| | | | | | | 113286231+ | |
| RAD52 | 1244 | chr12 | 916533 | 916638 | 15140 | chr12 | alt_3ss |
| | | | | | | 916484 | |
| | | | | | | 916532− | |
| REXO1 | 1245 | chr19 | 1817459 | 1817706 | 15141 | chr19 | alt_3ss |
| | | | | | | 1817330 | |
| | | | | | | 1817458− | |
| REXO1 | 1245 | chr19 | 1820181 | 1820263 | 15142 | chr19 | alt_3ss |
| | | | | | | 1820058 | |
| | | | | | | 1820180− | |
| RFX5 | 1246 | chr1 | 151344605 | 151344727 | 15143 | chr1 | alt_3ss |
| | | | | | | 151344537 | |
| | | | | | | 151344604− | |
| RUFY3 | 1252 | chr4 | 70789593 | 70793764 | 15144 | chr4 | alt_3ss |
| | | | | | | 70793765 | |
| | | | | | | 70793784+ | |
| SEMA3B | 1253 | chr3 | 50275054 | 50275176 | 15145 | chr3 | alt_3ss |
| | | | | | | 50275177 | |
| | | | | | | 50275301+ | |
| SEMA3F | 1254 | chr3 | 50184815 | 50185402 | 15146 | chr3 | alt_3ss |
| | | | | | | 50185403 | |
| | | | | | | 50185442+ | |
| SH2D3A | 1255 | chr19 | 6754449 | 6754615 | 15147 | chr19 | alt_3ss |
| | | | | | | 6754426 | |
| | | | | | | 6754448− | |
| SIRT3 | 1257 | chr11 | 230707 | 232982 | 15148 | chr11 | alt_3ss |
| | | | | | | 230553 | |
| | | | | | | 230706− | |
| SKIV2L | 1258 | chr6 | 31968539 | 31968622 | 15149 | chr6 | alt_3ss |
| | | | | | | 31968623 | |
| | | | | | | 31968685+ | |
| SLC25A13 | 1261 | chr7 | 96170150 | 96171471 | 15150 | chr7 | alt_3ss |
| | | | | | | 96170126 | |
| | | | | | | 96170149− | |
| SLC2A13 | 1264 | chr12 | 39830481 | 39864761 | 15898 | chr12 | alt_3ss |
| | | | | | | 39830229 | |
| | | | | | | 39830480− | |
| SLC30A10 | 1265 | chr1 | 219927144 | 219927800 | 15151 | chr1 | alt_3ss |
| | | | | | | 219927106 | |
| | | | | | | 219927143− | |
| SLC30A9 | 1266 | chr4 | 42065350 | 42066484 | 15152 | chr4 | alt_3ss |
| | | | | | | 42066485 | |
| | | | | | | 42066549+ | |
| SMTN | 1268 | chr22 | 31088114 | 31088453 | 15153 | chr22 | alt_3ss |
| | | | | | | 31088454 | |
| | | | | | | 31088512+ | |
| STXBP2 | 1272 | chr19 | 7645307 | 7645759 | 15154 | chr19 | alt_3ss |
| | | | | | | 7645760 | |
| | | | | | | 7646248+ | |
| SYNGAP1 | 1274 | chr6 | 33438920 | 33440552 | 15155 | chr6 | alt_3ss |
| | | | | | | 33440553 | |
| | | | | | | 33440728+ | |
| TBL2 | 1277 | chr7 | 73574805 | 73578419 | 15156 | chr7 | alt_3ss |
| | | | | | | 73574514 | |
| | | | | | | 73574804− | |
| TECPR2 | 1279 | chr14 | 102497121 | 102497468 | 15157 | chr14 | alt_3ss |
| | | | | | | 102497469 | |
| | | | | | | 102497569+ | |
| TNK2 | 1282 | chr3 | 195870379 | 195872275 | 15158 | chr3 | alt_3ss |
| | | | | | | 195870206 | |
| | | | | | | 195870378− | |
| TOE1 | 1283 | chr1 | 45342644 | 45342734 | 15159 | chr1 | alt_3ss |
| | | | | | | 45342735 | |
| | | | | | | 45342842+ | |
| TRMT2A | 1287 | chr22 | 20115555 | 20115671 | 15160 | chr22 | alt_3ss |
| | | | | | | 20115448 | |
| | | | | | | 20115554− | |
| TRPV4 | 1289 | chr12 | 109788743 | 109792362 | 15161 | chr12 | alt_3ss |
| | | | | | | 109788717 | |
| | | | | | | 109788742− | |
| UROD | 1293 | chr1 | 45012286 | 45012856 | 15162 | chr1 | alt_3ss |
| | | | | | | 45012857 | |
| | | | | | | 45012906+ | |
| WDR62 | 1295 | chr19 | 36066016 | 36066207 | 15163 | chr19 | alt_3ss |
| | | | | | | 36066208 | |
| | | | | | | 36066256+ | |
| WRAP53 | 1297 | chr17 | 7700830 | 7701426 | 15164 | chr17 | alt_3ss |
| | | | | | | 7701427 | |
| | | | | | | 7701458+ | |
| XPO1 | 1298 | chr2 | 61492251 | 61492324 | 15165 | chr2 | alt_3ss |
| | | | | | | 61492199 | |
| | | | | | | 61492250− | |

*alt_3ss refers to an alternative 3′ splice site of an intron, which is equivalent to the alternative 5′ splice site of the exon immediately downstream of the intron. coordinate of alternative intron. Region coordinate: coordinate of alternative intron. Event coordinate: coordinate of intronic region that is included due to alternative 3′ss or 5′ss selection. + or − refers to the strand in which the gene is located.

TABLE 5B

| | Genomic | | | | SEQ | Event | |
|---|---|---|---|---|---|---|---|
| | sequence | Region coordinate | | | ID | coordinate | Event |
| Gene | SEQ ID NO: | chrm | start | end | NO: | exon | type* |
| ABCA5 | 1116 | chr17 | 69248318 | 69249818 | 15166 | chr17 | alt_5ss |
| | | | | | | 69249819 | |
| | | | | | | 69249904− | |
| ABCA7 | 1117 | chr19 | 1063900 | 1064160 | 15167 | chr19 | alt_5ss |
| | | | | | | 1063864 | |
| | | | | | | 1063899+ | |
| ABCD1 | 1121 | chrX | 153736912 | 153737156 | 15168 | chrX | alt_5ss |
| | | | | | | 153736514 | |
| | | | | | | 153736911+ | |
| ABR | 1122 | chr17 | 1006170 | 1006774 | 15169 | chr17 | alt_5ss |
| | | | | | | 1006775 | |
| | | | | | | 1007164− | |
| ABR | 1122 | chr17 | 1006170 | 1006839 | 15170 | chr17 | alt_5ss |
| | | | | | | 1006840 | |
| | | | | | | 1007164− | |
| ABR | 1122 | chr17 | 1006170 | 1006904 | 15171 | chr17 | alt_5ss |
| | | | | | | 1006905 | |
| | | | | | | 1007164− | |
| ACAD9 | 1123 | chr3 | 128880018 | 128884652 | 15172 | chr3 | alt_5ss |
| | | | | | | 128879842 | |
| | | | | | | 128880017+ | |
| ACAP1 | 1124 | chr17 | 7350431 | 7350949 | 15173 | chr17 | alt_5ss |
| | | | | | | 7350238 | |
| | | | | | | 7350430+ | |
| ACTN4 | 1127 | chr19 | 38728382 | 38728995 | 15174 | chr19 | alt_5ss |
| | | | | | | 38728027 | |
| | | | | | | 38728381+ | |
| ADAMTS13 | 1129 | chr9 | 133430182 | 133432587 | 15175 | chr9 | alt_5ss |
| | | | | | | 133430102 | |
| | | | | | | 133430181+ | |
| AKR1E2 | 1130 | chr10 | 4847300 | 4847487 | 15176 | chr10 | alt_5ss |
| | | | | | | 4847231 | |
| | | | | | | 4847299+ | |
| ALG3 | 1132 | chr3 | 184245813 | 184248781 | 15177 | chr3 | alt_5ss |
| | | | | | | 184248782 | |
| | | | | | | 184248989− | |
| ALG3 | 1132 | chr3 | 184245813 | 184248781 | 15178 | chr3 | alt_5ss |
| | | | | | | 184248782 | |
| | | | | | | 184249225− | |
| ANKRD29 | 1133 | chr18 | 23617828 | 23619394 | 15179 | chr18 | alt_5ss |
| | | | | | | 23619395 | |
| | | | | | | 23619530− | |
| AP3M1 | 1137 | chr10 | 74124525 | 74126125 | 15180 | chr10 | alt_5ss |
| | | | | | | 74126126 | |
| | | | | | | 74126147− | |
| ASAP3 | 1140 | chr1 | 23434619 | 23435722 | 15181 | chr1 | alt_5ss |
| | | | | | | 23435723 | |
| | | | | | | 23435850− | |
| ATRX | 1141 | chrX | 77717244 | 77785844 | 15899 | chrX | alt_5ss |
| | | | | | | 77785845 | |
| | | | | | | 77785981− | |
| BBS4 | 1143 | chr15 | 72686565 | 72695176 | 15182 | chr15 | alt_5ss |
| | | | | | | 72686252 | |
| | | | | | | 72686564+ | |
| CATSPER1 | 1148 | chr11 | 66017175 | 66018787 | 15183 | chr11 | alt_5ss |
| | | | | | | 66018788 | |
| | | | | | | 66018826− | |
| CCDC40 | 1149 | chr17 | 80088190 | 80089763 | 15184 | chr17 | alt_5ss |
| | | | | | | 80088103 | |
| | | | | | | 80088189+ | |
| CCDC88B | 1150 | chr11 | 64342808 | 64343178 | 15185 | chr11 | alt_5ss |
| | | | | | | 64342681 | |
| | | | | | | 64342807+ | |
| CD4 | 1151 | chr12 | 6818651 | 6818846 | 15186 | chr12 | alt_5ss |
| | | | | | | 6818543 | |
| | | | | | | 6818650+ | |
| CES2 | 1153 | chr16 | 66935876 | 66938036 | 15187 | chr16 | alt_5ss |
| | | | | | | 66935712 | |
| | | | | | | 66935875+ | |
| CES2 | 1153 | chr16 | 66935977 | 66938036 | 15188 | chr16 | alt_5ss |
| | | | | | | 66935712 | |
| | | | | | | 66935976+ | |
| COL5A3 | 1158 | chr19 | 9992049 | 9992693 | 15189 | chr19 | alt_5ss |
| | | | | | | 9992694 | |
| | | | | | | 9992826− | |
| CPSF1 | 1159 | chr8 | 144398133 | 144398188 | 15190 | chr8 | alt_5ss |
| | | | | | | 144398189 | |
| | | | | | | 144398301− | |
| CSPP1 | 1160 | chr8 | 67064539 | 67074242 | 15191 | chr8 | alt_5ss |
| | | | | | | 67064497 | |
| | | | | | | 67064538+ | |
| CTH | 1162 | chr1 | 70411657 | 70415955 | 15192 | chr1 | alt_5ss |
| | | | | | | 70411584 | |
| | | | | | | 70411656+ | |
| DAB2IP | 1164 | chr9 | 121678918 | 121699324 | 15193 | chr9 | alt_5ss |
| | | | | | | 121678782 | |
| | | | | | | 121678917+ | |
| DBN1 | 1165 | chr5 | 177467818 | 177467995 | 15194 | chr5 | alt_5ss |
| | | | | | | 177467996 | |
| | | | | | | 177468107− | |
| DBN1 | 1165 | chr5 | 177467818 | 177468051 | 15195 | chr5 | alt_5ss |
| | | | | | | 177468052 | |
| | | | | | | 177468107− | |
| DDX51 | 1166 | chr12 | 132141021 | 132141102 | 15196 | chr12 | alt_5ss |
| | | | | | | 132141103 | |
| | | | | | | 132141274− | |
| DEAF1 | 1167 | chr11 | 691599 | 694678 | 15197 | chr11 | alt_5ss |
| | | | | | | 694679 | |
| | | | | | | 694758− | |
| DGKE | 1168 | chr17 | 56856666 | 56858593 | 15198 | chr17 | alt_5ss |
| | | | | | | 56856626 | |
| | | | | | | 56856665+ | |
| DMKN | 1170 | chr19 | 35497539 | 35498597 | 15199 | chr19 | alt_5ss |
| | | | | | | 35498598 | |
| | | | | | | 35498715− | |
| DOCK8 | 1172 | chr9 | 215417 | 271626 | 15900 | chr9 | alt_5ss |
| | | | | | | 215030 | |
| | | | | | | 215416+ | |
| DOK5 | 1173 | chr20 | 54476186 | 54554932 | 15901 | chr20 | alt_5ss |
| | | | | | | 54476013 | |
| | | | | | | 54476185+ | |
| EIF2B5 | 1174 | chr3 | 184140222 | 184140417 | 15200 | chr3 | alt_5ss |
| | | | | | | 184140158 | |
| | | | | | | 184140221+ | |
| ELAC2 | 1175 | chr17 | 13017122 | 13017566 | 15201 | chr17 | alt_5ss |
| | | | | | | 13017567 | |
| | | | | | | 13017702− | |
| ELP1 | 1176 | chr9 | 108896645 | 108896890 | 15202 | chr9 | alt_5ss |
| | | | | | | 108896891 | |
| | | | | | | 108896952− | |
| ETHE1 | 1181 | chr19 | 43526660 | 43526936 | 15203 | chr19 | alt_5ss |
| | | | | | | 43526937 | |
| | | | | | | 43527096− | |
| FANCA | 1182 | chr16 | 89739554 | 89739705 | 15204 | chr16 | alt_5ss |
| | | | | | | 89739706 | |
| | | | | | | 89739993− | |
| FASTK | 1183 | chr7 | 151079923 | 151080436 | 15205 | chr7 | alt_5ss |
| | | | | | | 151080437 | |
| | | | | | | 151080684− | |
| FASTK | 1183 | chr7 | 151079923 | 151080208 | 15206 | chr7 | alt_5ss |
| | | | | | | 151080209 | |
| | | | | | | 151080684− | |
| FPGS | 1185 | chr9 | 127807776 | 127808233 | 15207 | chr9 | alt_5ss |
| | | | | | | 127807689 | |
| | | | | | | 127807775+ | |
| GAS8 | 1187 | chr16 | 90035880 | 90036380 | 15208 | chr16 | alt_5ss |
| | | | | | | 90035688 | |
| | | | | | | 90035879+ | |
| GCK | 1188 | chr7 | 44145731 | 44146073 | 15209 | chr7 | alt_5ss |
| | | | | | | 44146074 | |
| | | | | | | 44146462− | |
| GGA1 | 1189 | chr22 | 37609155 | 37614189 | 15210 | chr22 | alt_5ss |
| | | | | | | 37608904 | |
| | | | | | | 37609154+ | |
| GON4L | 1193 | chr1 | 155773211 | 155774962 | 15211 | chr1 | alt_5ss |
| | | | | | | 155774963 | |
| | | | | | | 155775001− | |
| HELQ | 1196 | chr4 | 83453946 | 83455268 | 15212 | chr4 | alt_5ss |
| | | | | | | 83455269 | |
| | | | | | | 83455396− | |
| HPS1 | 1198 | chr10 | 98431292 | 98433939 | 15213 | chr10 | alt_5ss |
| | | | | | | 98433940 | |
| | | | | | | 98433982− | |
| IL17RC | 1202 | chr3 | 9924138 | 9924231 | 15214 | chr3 | alt_5ss |
| | | | | | | 9924021 | |
| | | | | | | 9924137+ | |
| INVS | 1204 | chr9 | 100292874 | 100292946 | 15215 | chr9 | alt_5ss |
| | | | | | | 100292437 | |
| | | | | | | 100292873+ | |
| IRF3 | 1205 | chr19 | 49660829 | 49661449 | 15216 | chr19 | alt_5ss |
| | | | | | | 49661450 | |
| | | | | | | 49661947− | |
| IRF3 | 1205 | chr19 | 49660845 | 49661449 | 15217 | chr19 | alt_5ss |
| | | | | | | 49661450 | |
| | | | | | | 49661947− | |
| KLKB1 | 1207 | chr4 | 186250434 | 186251218 | 15218 | chr4 | alt_5ss |
| | | | | | | 186250403 | |
| | | | | | | 186250433+ | |
| LAMC3 | 1209 | chr9 | 131085872 | 131087475 | 15219 | chr9 | alt_5ss |
| | | | | | | 131085724 | |
| | | | | | | 131085871+ | |
| LDAH | 1210 | chr2 | 20685658 | 20686837 | 15220 | chr2 | alt_5ss |
| | | | | | | 20686838 | |
| | | | | | | 20686906− | |
| LIMS2 | 1211 | chr2 | 127673744 | 127674620 | 15221 | chr2 | alt_5ss |
| | | | | | | 127674621 | |
| | | | | | | 127675013− | |
| MALT1 | 1212 | chr18 | 58734567 | 58735201 | 15222 | chr18 | alt_5ss |
| | | | | | | 58734382 | |
| | | | | | | 58734566+ | |
| MAPK13 | 1214 | chr6 | 36131158 | 36131270 | 15223 | chr6 | alt_5ss |
| | | | | | | 36130702 | |
| | | | | | | 36131157+ | |
| MCAT | 1215 | chr22 | 43141250 | 43142891 | 15224 | chr22 | alt_5ss |
| | | | | | | 43142892 | |
| | | | | | | 43142925− | |
| MCEE | 1216 | chr2 | 71124544 | 71129716 | 15225 | chr2 | alt_5ss |
| | | | | | | 71129717 | |
| | | | | | | 71130179− | |
| MPI | 1217 | chr15 | 74890202 | 74890526 | 15226 | chr15 | alt_5ss |
| | | | | | | 74890090 | |
| | | | | | | 74890201+ | |
| MSTO1 | 1218 | chr1 | 155611356 | 155611548 | 15227 | chr1 | alt_5ss |
| | | | | | | 155611292 | |
| | | | | | | 155611355+ | |
| NLE1 | 1220 | chr17 | 35139315 | 35139819 | 15228 | chr17 | alt_5ss |
| | | | | | | 35139820 | |
| | | | | | | 35139848− | |
| NLRC5 | 1221 | chr16 | 57028204 | 57028301 | 15229 | chr16 | alt_5ss |
| | | | | | | 57028156 | |
| | | | | | | 57028203+ | |
| NOM1 | 1222 | chr7 | 156960254 | 156962150 | 15230 | chr7 | alt_5ss |
| | | | | | | 156960175 | |
| | | | | | | 156960253+ | |
| NPR1 | 1225 | chr1 | 153682198 | 153682497 | 15231 | chr1 | alt_5ss |
| | | | | | | 153681840 | |
| | | | | | | 153682197+ | |
| OSGEP | 1227 | chr14 | 20448812 | 20448901 | 15232 | chr14 | alt_5ss |
| | | | | | | 20448902 | |
| | | | | | | 20448963− | |
| PABPC4 | 1228 | chr1 | 39570003 | 39571096 | 15233 | chr1 | alt_5ss |
| | | | | | | 39571097 | |
| | | | | | | 39571233− | |
| PCOLCE | 1229 | chr7 | 100604503 | 100605090 | 15234 | chr7 | alt_5ss |
| | | | | | | 100604218 | |
| | | | | | | 100604502+ | |
| PLD2 | 1234 | chr17 | 4818407 | 4818493 | 15235 | chr17 | alt_5ss |
| | | | | | | 4818386 | |
| | | | | | | 4818406+ | |
| PNPO | 1236 | chr17 | 47942052 | 47943305 | 15236 | chr17 | alt_5ss |
| | | | | | | 47941814 | |
| | | | | | | 47942051+ | |
| POLE | 1237 | chr12 | 132672323 | 132672579 | 15237 | chr12 | alt_5ss |
| | | | | | | 132672580 | |
| | | | | | | 132672626− | |
| PON2 | 1238 | chr7 | 95412478 | 95416171 | 15238 | chr7 | alt_5ss |
| | | | | | | 95416172 | |
| | | | | | | 95416241− | |
| PYCR1 | 1243 | chr17 | 81936862 | 81936969 | 15239 | chr17 | alt_5ss |
| | | | | | | 81936970 | |
| | | | | | | 81937179− | |
| RAD52 | 1244 | chr12 | 927264 | 929763 | 15240 | chr12 | alt_5ss |
| | | | | | | 929764 | |
| | | | | | | 929818− | |
| RAD52 | 1244 | chr12 | 914122 | 914410 | 15241 | chr12 | alt_5ss |
| | | | | | | 914411 | |
| | | | | | | 914430− | |
| RHBDF2 | 1247 | chr17 | 76474535 | 76474659 | 15242 | chr17 | alt_5ss |
| | | | | | | 76474660 | |
| | | | | | | 76474729− | |
| RMND1 | 1248 | chr6 | 151433231 | 151436419 | 15243 | chr6 | alt_5ss |
| | | | | | | 151436420 | |
| | | | | | | 151436445− | |
| ROBO3 | 1249 | chr11 | 124876933 | 124877160 | 15244 | chr11 | alt_5ss |
| | | | | | | 124876461 | |
| | | | | | | 124876932+ | |
| ROBO3 | 1249 | chr11 | 124877753 | 124877936 | 15245 | chr11 | alt_5ss |
| | | | | | | 124877659 | |
| | | | | | | 124877752+ | |
| RPGRIP1 | 1250 | chr14 | 21321630 | 21321853 | 15246 | chr14 | alt_5ss |
| | | | | | | 21321403 | |
| | | | | | | 21321629+ | |
| RTTN | 1251 | chr18 | 70060043 | 70065756 | 15247 | chr18 | alt_5ss |
| | | | | | | 70065757 | |
| | | | | | | 70065828− | |
| SIK3 | 1256 | chr11 | 116868090 | 116870140 | 15248 | chr11 | alt_5ss |
| | | | | | | 116870141 | |
| | | | | | | 116870330− | |
| SLC12A7 | 1259 | chr5 | 1075491 | 1075890 | 15249 | chr5 | alt_5ss |
| | | | | | | 1075891 | |
| | | | | | | 1076137− | |
| SLC22A3 | 1260 | chr6 | 160349059 | 160397978 | 15902 | chr6 | alt_5ss |
| | | | | | | 160348849 | |
| | | | | | | 160349058+ | |
| SLC25A37 | 1262 | chr8 | 23566814 | 23568321 | 15250 | chr8 | alt_5ss |
| | | | | | | 23566337 | |
| | | | | | | 23566813+ | |
| SLC27A5 | 1263 | chr19 | 58499221 | 58499394 | 15251 | chr19 | alt_5ss |
| | | | | | | 58499395 | |
| | | | | | | 58499491− | |
| SMPD1 | 1267 | chr11 | 6392197 | 6393215 | 15252 | chr11 | alt_5ss |
| | | | | | | 6392157 | |
| | | | | | | 6392196+ | |
| SNRPB | 1269 | chr20 | 2465820 | 2467191 | 15253 | chr20 | alt_5ss |
| | | | | | | 2467192 | |
| | | | | | | 2467606− | |
| SP140 | 1270 | chr2 | 230310212 | 230310742 | 15254 | chr2 | alt_5ss |
| | | | | | | 230310040 | |
| | | | | | | 230310211+ | |
| STAMBP | 1271 | chr2 | 73845037 | 73845166 | 15255 | chr2 | alt_5ss |
| | | | | | | 73844889 | |
| | | | | | | 73845036+ | |
| SULF2 | 1273 | chr20 | 47665954 | 47666087 | 15256 | chr20 | alt_5ss |
| | | | | | | 47666088 | |
| | | | | | | 47666259− | |
| SYNJ2 | 1275 | chr6 | 158062222 | 158063790 | 15257 | chr6 | alt_5ss |
| | | | | | | 158062165 | |
| | | | | | | 158062221+ | |
| TAZ | 1276 | chrX | 154413359 | 154413481 | 15258 | chrX | alt_5ss |
| | | | | | | 154413253 | |
| | | | | | | 154413358+ | |
| TDRD7 | 1278 | chr9 | 97478636 | 97480827 | 15259 | chr9 | alt_5ss |
| | | | | | | 97478574 | |
| | | | | | | 97478635+ | |
| TFB1M | 1280 | chr6 | 155311340 | 155314116 | 15260 | chr6 | alt_5ss |
| | | | | | | 155314117 | |
| | | | | | | 155314295− | |
| TM6SF1 | 1281 | chr15 | 83119735 | 83121920 | 15261 | chr15 | alt_5ss |
| | | | | | | 83119682 | |
| | | | | | | 83119734+ | |
| TOR1B | 1284 | chr9 | 129804480 | 129807187 | 15262 | chr9 | alt_5ss |
| | | | | | | 129804339 | |
| | | | | | | 129804479+ | |
| TRAPPC6B | 1285 | chr14 | 39158403 | 39159449 | 15263 | chr14 | alt_5ss |
| | | | | | | 39159450 | |
| | | | | | | 39159482− | |
| TRIP4 | 1286 | chr15 | 64388049 | 64393945 | 15264 | chr15 | alt_5ss |
| | | | | | | 64387965 | |
| | | | | | | 64388048+ | |
| TRMU | 1288 | chr22 | 46336107 | 46337778 | 15265 | chr22 | alt_5ss |
| | | | | | | 46335847 | |
| | | | | | | 46336106+ | |
| TTC19 | 1290 | chr17 | 16000383 | 16001914 | 15266 | chr17 | alt_5ss |
| | | | | | | 16000246 | |
| | | | | | | 16000382+ | |
| TYMP | 1291 | chr22 | 50526739 | 50526897 | 15267 | chr22 | alt_5ss |
| | | | | | | 50526898 | |
| | | | | | | 50527164− | |
| UMPS | 1292 | chr3 | 124738444 | 124740023 | 15268 | chr3 | alt_5ss |
| | | | | | | 124738240 | |
| | | | | | | 124738443+ | |
| WDR11 | 1294 | chr10 | 120851528 | 120852523 | 15269 | chr10 | alt_5ss |
| | | | | | | 120851507 | |
| | | | | | | 120851527+ | |
| WIPI1 | 1296 | chr17 | 68433576 | 68434419 | 15270 | chr17 | alt_5ss |
| | | | | | | 68434420 | |
| | | | | | | 68434555− | |
| YY1AP1 | 1299 | chr1 | 155688202 | 155688578 | 15271 | chr1 | alt_5ss |
| | | | | | | 155688579 | |
| | | | | | | 155688929− | |
| ZC3H14 | 1300 | chr14 | 88607395 | 88609266 | 15272 | chr14 | alt_5ss |
| | | | | | | 88607364 | |
| | | | | | | 88607394+ | |
| ZC3H14 | 1300 | chr14 | 88607426 | 88609266 | 15273 | chr14 | alt_5ss |
| | | | | | | 88607364 | |
| | | | | | | 88607425+ | |

*alt_5ss refers to an alternative 5′ splice site of an intron, which is equivalent to the alternative 3′ splice site of the exon immediately upstream of the intron. Region coordinate: coordinate of alternative intron. Event coordinate: coordinate of intronic region that is included due to alternative 3′ss or 5′ss selection. + or − refers to the strand in which the gene is located.

TABLE 5C

| | Genomic | | | | SEQ | | | Event |
|---|---|---|---|---|---|---|---|---|
| | sequence | | | Region | ID | Event | Event | size |
| Gene | SEQ ID NO: | Disease | Genetics | coordinate | NO: | coordinate | type* | (bp) |
| GRN | | GRN-related | Haploinsufficiency | chr17 | | chr17 | Alt | −20 |
| | | frontotemporal | | 44350228 | | 44350442 | 3′ss | |
| | | dementia | | 44350577 | | 44350461 | | |
| SHANK3 | | Phelan- | Haploinsufficiency | chr22 | | chr22 | Alt | 124 |
| | | McDermid | | 50705033 | | 50705948 | 3′ss | |
| | | syndrome; | | 50706149 | | 50706071 | | |
| | | Autism | | | | | | |
| | | spectrum | | | | | | |
| | | disorder | | | | | | |
| PRPF3 | 1241 | Retinitis | Haploinsufficiency | chr1 | | chr1 | Alt | 58 |
| | | pigmentosa | | 150344263 | | 150344376 | 3′ss | |
| | | 18 | | 150344375 | | 150344433 | | |
| PRPF4 | 1242 | Retinitis | Haploinsufficiency | chr9 | | chr9 | Alt | −49 |
| | | pigmentosa | | 113290540 | | 113290540 | 5′ss | |
| | | 70 | | 113290699 | | 113290588 | | |
| | | | | chr9 | 15274 | chr9 | Alt | 39 |
| | | | | 113284390 | | 113286193 | 3′ss | |
| | | | | 113286192 | | 113286231 | | |
| SYNG | 1274 | AD MENTAL | Haploinsufficiency | chr6 | | chr6 | Alt | −37 |
| AP1 | | RETARDATION | | 33438883 | | 33438883 | 5′ss | |
| | | 5 | | 33440728 | | 33438919 | | |
| | | | | chr6 | | chr6 | Alt | 179 |
| | | | | 33438920 | | 33440550 | 3′ss | |
| | | | | 33440549 | | 33440728 | | |
| | | | | chr6 | 15275 | chr6 | Alt | 176 |
| | | | | 33438920 | | 33440553 | 3′ss | |
| | | | | 33440552 | | 33440728 | | |
| ABCA | | Stargardt | Autosomal | chr1 | | chr1 | Alt | −41 |
| 4 | | disease | recessive | 94019669 | | 94019583 | 3′ss | |
| | | | | 94021239 | | 94019668 | | |
| DNAJ | | Pathway/EYE | N/A | chr2 | | chr2 | Alt | 77 |
| B2 | | | | 219279976 | | 219279899 | 5′ss | |
| | | | | 219280577 | | 219279975 | | |
| | | | | chr2 | | chr2 | Alt | −28 |
| | | | | 219282930 | | 219283133 | 3′ss | |
| | | | | 219283160 | | 219283160 | | |
| CLPX | | Pathway/EYE | N/A | chr15 | | chr15 | Alt | 66 |
| | | | | 65155857 | | 65156778 | 5′ss | |
| | | | | 65156777 | | 65156843 | | |
| HSPA | | Pathway/EYE | N/A | chr5 | | chr5 | Alt | 43 |
| 9 | | | | 138574170 | | 138574127 | 3′ss | |
| | | | | 138575237 | | 138574169 | | |
| SLC4 | | Fuch's corneal | Autosomal | chr20 | | chr20 | Alt | −49 |
| A11 | | dystrophy | recessive | 3229214 | | 3229214 | 3′ss | |
| | | | | 3229345 | | 3229263 | | |
| | | | | chr20 | | chr20 | Alt | −70 |
| | | | | 3229707 | | 3229707 | 3′ss | |
| | | | | 3230186 | | 3229776 | | |
| OPTN | | Pathway | N/A | chr10 | | chr10 | Alt | 94 |
| | | | | 13109289 | | 13110180 | 3′ss | |
| | | | | 13110179 | | 13110273 | | |
| eIF2B | 1174 | Pathway | N/A | chr3 | 15276 | chr3 | Alt | 64 |
| 5 | | | | 184140222 | | 184140158 | 5′ss | |
| | | | | 184140417 | | 184140221 | | |
| | | | | chr3 | | chr3 | Alt | 15 |
| | | | | 184142395 | | 184142379 | 5′ss | |
| | | | | 184142501 | | 184142394 | | |
| SIRT3 | 1257 | Pathway | N/A | chr11 | | chr11 | Alt | 89 |
| | | | | 230642 | | 230553 | 3′ss | |
| | | | | 232982 | | 230641 | | |
| | | | | chr11 | 15277 | chr11 | Alt | 154 |
| | | | | 230707 | | 230553 | 3′ss | |
| | | | | 232982 | | 230706 | | |
| STAT | | Pathway | N/A | chr17 | | chr17 | Alt | 119 |
| 3 | | | | 42323355 | | 42323453 | 5′ss | |
| | | | | 42323452 | | 42323572 | | |
| KCNQ | | early-onset | Haploinsufficiency | chr20 | | chr20 | Alt | −56 |
| 2 | | epileptic | | 63444835 | | 63445238 | 5′ss | |
| | | encephalopathy | | 63445293 | | 63445293 | | |
| CACN | | Spinocerebellar | Haploinsufficiency ? | chr17 | | chr17 | Alt | 123 |
| A1G | | ataxia 42 [AD] | | 50569804 | | 50571755 | 3′ss | |
| | | | | 50571754 | | 50571877 | | |
| | | | | chr17 | | chr17 | Alt | −49 |
| | | | | 50617930 | | 50618048 | 3′ss | |
| | | | | 50618096 | | 50618096 | | |
| GBA | | Parkinson | Haploinsufficiency | chr1 | | chr1 | Alt | −115 |
| | | disease | | 155239963 | | 155239963 | 3′ss | |
| | | | | 155240629 | | 155240077 | | |
| | | | | chr1 | | chr1 | Alt | 453 |
| | | | | 155240078 | | 155240177 | 5′ss | |
| | | | | 155240176 | | 155240629 | | |
| KL | | Pathway | N/A | chr13 | | chr13 | Alt | 50 |
| | | | | 33055366 | | 33055316 | 5′ss | |
| | | | | 33060678 | | 33055365 | | |
| AKT2 | | Pathway | N/A | chr19 | | chr19 | Alt | 117 |
| | | | | 40235468 | | 40235351 | 3′ss | |
| | | | | 40235889 | | 40235467 | | |
| EIF2A | | Pathway | N/A | chr2 | | chr2 | Alt | −44 |
| K3 | | | | 88591009 | | 88591009 | 3′ss | |
| | | | | 88593271 | | 88591052 | | |
| SEMA | | Pathway | N/A | chr1 | | chr1 | Alt | 17 |
| 4A | | | | 156158487 | | 156158702 | 3′ss | |
| | | | | 156158701 | | 156158718 | | |
| SEMA | 1253 | Pathway | N/A | chr3 | 15278 | chr3 | Alt | 125 |
| 3B | | | | 50275054 | | 50275177 | 3′ss | |
| | | | | 50275176 | | 50275301 | | |
| SEMA | 1254 | Pathway | N/A | chr3 | 15279 | chr3 | Alt | 40 |
| 3F | | | | 50184815 | | 50185403 | 3′ss | |
| | | | | 50185402 | | 50185442 | | |
| | | | | chr3 | | chr3 | Alt | -19 |
| | | | | 50184796 | | 50184796 | 5′ss | |
| | | | | 50185442 | | 50184814 | | |
| SLC4 | | Corneal | Haploinsufficiency | chr20 | | chr20 | Alt | −70 |
| A11 | | dystrophy, | | 3229707 | | 3229707 | 3′ss | |
| | | Fuchs | | 3230186 | | 3229776 | | |
| | | endothelial, | | | | | | |
| | | 4 | | | | | | |
| JAG1 | | Alagille | Haploinsufficiency | chr20 | | chr20 | Alt | 354 |
| | | syndrome | | 10647104 | | 10647606 | 5′ss | |
| | | | | 10647605 | | 10647959 | | |
| CHD2 | | Epileptic | Haploinsufficiency | chr15 | | chr15 | Alt | 86 |
| | | encephalopathy, | | 92974951 | | 92978148 | 3′ss | |
| | | childhood-onset | | 92978147 | | 92978233 | | |
| MCEE | 1216 | Methylmalonyl- | Autosomal | chr2 | 15280 | chr2 | Alt | 463 |
| | | CoA epimerase | recessive | 71124544 | | 71129717 | 5′ss | |
| | | deficiency | | 71129716 | | 71130179 | | |
| TRAP | 1285 | Neurodevelopm | Autosomal | chr14 | 15281 | chr14 | Alt | 33 |
| PC6B | | ental disorder | recessive | 39158403 | | 39159450 | 5′ss | |
| | | with microcephaly, | | 39159449 | | 39159482 | | |
| | | epilepsy, and | | | | | | |
| | | brain atrophy | | | | | | |
| AP3B | 1136 | Epileptic | Autosomal | chr15 | 15282 | chr15 | Alt | 62 |
| 2 | | encephalopathy, | recessive | 82663296 | | 82663234 | 3′ss | |
| | | early infantile, | | 82663559 | | 82663295 | | |
| | | 48 | | | | | | |
| TTC19 | 1290 | Mitochondrial | Autosomal | chr17 | 15283 | chr17 | Alt | 137 |
| | | complex III | recessive | 16000383 | | 16000246 | 5′ss | |
| | | deficiency, | | 16001914 | | 16000382 | | |
| | | nuclear type 2 | | | | | | |
| DMTF | | Tumor | N/A | chr7 | | chr7 | Alt | 172 |
| 1 | | suppressor | | 87181342 | | 87182056 | 3′ss | |
| | | | | 87182055 | | 87182227 | | |
| | | | | chr7 | | chr7 | Alt | 211 |
| | | | | 87181342 | | 87182017 | 3′ss | |
| | | | | 87182016 | | 87182227 | | |
| HDAC | 1195 | Tumor | N/A | chr5 | 15284 | chr5 | Alt | 25 |
| 3 | | suppressor | | 141628213 | | 141628188 | 3′ss | |
| | | | | 141628558 | | 141628212 | | |
| DAB2I | 1164 | Tumor | N/A | chr9 | 15285 | chr9 | Alt | 136 |
| P | | suppressor | | 121678918 | | 121678782 | 5′ss | |
| | | | | 121699324 | | 121678917 | | |
| CD4 | 1151 | Tumor | N/A | chr12 | 15286 | chr12 | Alt | 108 |
| | | suppressor | | 6818651 | | 6818543 | 5′ss | |
| | | | | 6818846 | | 6818650 | | |
| TP53B | | Tumor | N/A | chr1 | | chr1 | Alt | 302 |
| P2 | | suppressor | | 223802345 | | 223802429 | 5′ss | |
| | | | | 223802428 | | 223802730 | | |
| BAP1 | | Tumor | N/A | chr3 | | chr3 | Alt | 136 |
| | | suppressor | | 52406459 | | 52406323 | 3′ss | |
| | | | | 52406828 | | 52406458 | | |
| | | | | | | chr3 | Alt | 82 |
| | | | | | | 52406377 | 3′ss | |
| | | | | | | 52406458 | | |
| CHFR | 1154 | Tumor | N/A | chr12 | | chr12 | Alt | 68 |
| | | suppressor | | 132848224 | | 132848156 | 3′ss | |
| | | | | 132848640 | | 132848223 | | |
| IRF3 | 1205 | Tumor | N/A | chr19 | 15287 | chr19 | Alt | 498 |
| | | suppressor | | 49660829 | | 49661450 | 5′ss | |
| | | | | 49661449 | | 49661947 | | |
| SIRT2 | | Tumor | N/A | chr19 | | chr19 | Alt | 471 |
| | | suppressor | | 38880174 | | 38879703 | 3′ss | |
| | | | | 38880684 | | 38880173 | | |
| RBL1 | | Tumor | N/A | chr20 | | chr20 | Alt | 138 |
| | | suppressor | | 37061270 | | 37061946 | 5′ss | |
| | | | | 37061945 | | 37062083 | | |

*Alt 5ss refers to an alternative 5′ splice site of an intron, which is equivalent to the alternative 3′ splice site of the exon immediately upstream of the intron. Alt 3ss refers to an alternative 3′ splice site of an intron, which is equivalent to the alternative 5′ splice site of the exon immediately downstream of the intron. Region coordinate: coordinate of alternative intron. Event coordinate: coordinate of intronic region that is included due to alternative 3′ss or 5′ss selection.

In some embodiments, the ASOs disclosed herein target a NSAE pre-mRNA transcribed from a ABCA4 genomic sequence. In some embodiments, the ASO targets a NSAE pre-mRNA transcript from a ABCA4 genomic sequence comprising a NSAE exon. In some embodiments, the ASO targets a NSAE pre-mRNA transcript from a ABCA4 genomic sequence comprising an intron flanking the 3′ splice site of the NSAE exon and an intron flanking the 5′ splice site of a NSAE exon. In some embodiments, the ASO targets a NSAE pre-mRNA transcript of ABCA4. In some embodiments, the ASO targets a ABCA4 pre-mRNA sequence comprising a NSAE exon. In some embodiments, the ASO targets a ABCA4 pre-mRNA sequence comprising an intron flanking the 3′ splice site of the NSAE exon. In some embodiments, the ASO targets a ABCA4 pre-mRNA sequence comprising an intron flanking the 5′ splice site of the NSAE exon.

In some embodiments, the ABCA4 pre-mRNA transcript is encoded by a genetic sequence with at least about 80%, 85%, 90%, 95%, 96%, 97%, 98%, 99% or 100% sequence identity to the Ensembl reference number ENSG00000198691.13 or a complement thereof. In some embodiments, the ABCA4 pre-mRNA transcript comprises a sequence with at least about 80%, 85%, 90%, 95%, 96%, 97%, 98%, 99% or 100% sequence identity to a ABCA4 pre-mRNA transcript or a complement thereof described herein.

In some embodiments, the targeted portion of the ABCA4 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the ABCA4 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a AKT2 genomic sequence. In some embodiments, the ASO targets a NSAE pre-mRNA transcript from a AKT2 genomic sequence comprising a NSAE exon. In some embodiments, the ASO targets a NSAE pre-mRNA transcript from a AKT2 genomic sequence comprising an intron flanking the 3′ splice site of the NSAE exon and an intron flanking the 5′ splice site of a NSAE exon. In some embodiments, the ASO targets a NSAE pre-mRNA transcript of AKT2. In some embodiments, the ASO targets a AKT2 pre-mRNA sequence comprising a NSAE exon. In some embodiments, the ASO targets a AKT2 pre-mRNA sequence comprising an intron flanking the 3′ splice site of the NSAE exon. In some embodiments, the ASO targets a AKT2 pre-mRNA sequence comprising an intron flanking the 5′ splice site of the NSAE exon.

In some embodiments, the AKT2 pre-mRNA transcript is encoded by a genetic sequence with at least about 80%, 85%, 90%, 95%, 96%, 97%, 98%, 99% or 100% sequence identity to the Ensembl reference number ENSG00000105221.18 or a complement thereof. In some embodiments, the AKT2 pre-mRNA transcript comprises a sequence with at least about 80%, 85%, 90%, 95%, 96%, 97%, 98%, 99% or 100% sequence identity to a AKT2 pre-mRNA transcript or a complement thereof described herein.

In some embodiments, the targeted portion of the AKT2 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the AKT2 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a BAP1 genomic sequence. In some embodiments, the ASO targets a NSAE pre-mRNA transcript from a BAP1 genomic sequence comprising a NSAE exon. In some embodiments, the ASO targets a NSAE pre-mRNA transcript from a BAP1 genomic sequence comprising an intron flanking the 3′ splice site of the NSAE exon and an intron flanking the 5′ splice site of a NSAE exon. In some embodiments, the ASO targets a NSAE pre-mRNA transcript of BAP1. In some embodiments, the ASO targets a BAP1 pre-mRNA sequence comprising a NSAE exon. In some embodiments, the ASO targets a BAP1 pre-mRNA sequence comprising an intron flanking the 3′ splice site of the NSAE exon. In some embodiments, the ASO targets a BAP1 pre-mRNA sequence comprising an intron flanking the 5′ splice site of the NSAE exon.

In some embodiments, the BAP1 pre-mRNA transcript is encoded by a genetic sequence with at least about 80%, 85%, 90%, 95%, 96%, 97%, 98%, 99% or 100% sequence identity to the Ensembl reference number ENSG00000163930.10 or a complement thereof. In some embodiments, the BAP1 pre-mRNA transcript comprises a sequence with at least about 80%, 85%, 90%, 95%, 96%, 97%, 98%, 99% or 100% sequence identity to a BAP1 pre-mRNA transcript or a complement thereof described herein. In some embodiments, the targeted portion of the BAP1 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the BAP1 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a CACNA1G genomic sequence. In some embodiments, the ASO targets a NSAE pre-mRNA transcript from a CACNA1G genomic sequence comprising a NSAE exon. In some embodiments, the ASO targets a NSAE pre-mRNA transcript from a CACNA1G genomic sequence comprising an intron flanking the 3′ splice site of the NSAE exon and an intron flanking the 5′ splice site of a NSAE exon. In some embodiments, the ASO targets a NSAE pre-mRNA transcript of CACNA1G. In some embodiments, the ASO targets a CACNA1G pre-mRNA sequence comprising a NSAE exon. In some embodiments, the ASO targets a CACNA1G pre-mRNA sequence comprising an intron flanking the 3′ splice site of the NSAE exon. In some embodiments, the ASO targets a CACNA1G pre-mRNA sequence comprising an intron flanking the 5′ splice site of the NSAE exon.

In some embodiments, the CACNA1G pre-mRNA transcript is encoded by a genetic sequence with at least about 80%, 85%, 90%, 95%, 96%, 97%, 98%, 99% or 100% sequence identity to the Ensembl reference number ENSG00000006283.18 or a complement thereof. In some embodiments, the CACNA1G pre-mRNA transcript comprises a sequence with at least about 80%, 85%, 90%, 95%, 96%, 97%, 98%, 99% or 100% sequence identity to a CACNA1G pre-mRNA transcript or a complement thereof described herein. In some embodiments, the targeted portion of the CACNA1G pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the CACNA1G pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a CHD2 genomic sequence. In some embodiments, the ASO targets a NSAE pre-mRNA transcript from a CHD2 genomic sequence comprising a NSAE exon. In some embodiments, the ASO targets a NSAE pre-mRNA transcript from a CHD2 genomic sequence comprising an intron flanking the 3′ splice site of the NSAE exon and an intron flanking the 5′ splice site of a NSAE exon. In some embodiments, the ASO targets a NSAE pre-mRNA transcript of CHD2. In some embodiments, the ASO targets a CHD2 pre-mRNA sequence comprising a NSAE exon. In some embodiments, the ASO targets a CHD2 pre-mRNA sequence comprising an intron flanking the 3′ splice site of the NSAE exon. In some embodiments, the ASO targets a CHD2 pre-mRNA sequence comprising an intron flanking the 5′ splice site of the NSAE exon.

In some embodiments, the CHD2 pre-mRNA transcript is encoded by a genetic sequence with at least about 80%, 85%, 90%, 95%, 96%, 97%, 98%, 99% or 100% sequence identity to the Ensembl reference number ENSG00000173575.22 or a complement thereof. In some embodiments, the CHD2 pre-mRNA transcript comprises a sequence with at least about 80%, 85%, 90%, 95%, 96%, 97%, 98%, 99% or 100% sequence identity to a CHD2 pre-mRNA transcript or a complement thereof described herein. In some embodiments, the targeted portion of the CHD2 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the CHD2 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a CLPX genomic sequence. In some embodiments, the ASO targets a NSAE pre-mRNA transcript from a CLPX genomic sequence comprising a NSAE exon. In some embodiments, the ASO targets a NSAE pre-mRNA transcript from a CLPX genomic sequence comprising an intron flanking the 3′ splice site of the NSAE exon and an intron flanking the 5′ splice site of a NSAE exon. In some embodiments, the ASO targets a NSAE pre-mRNA transcript of CLPX. In some embodiments, the ASO targets a CLPX pre-mRNA sequence comprising a NSAE exon. In some embodiments, the ASO targets a CLPX pre-mRNA sequence comprising an intron flanking the 3′ splice site of the NSAE exon. In some embodiments, the ASO targets a CLPX pre-mRNA sequence comprising an intron flanking the 5′ splice site of the NSAE exon.

In some embodiments, the CLPX pre-mRNA transcript is encoded by a genetic sequence with at least about 80%, 85%, 90%, 95%, 96%, 97%, 98%, 99% or 100% sequence identity to the Ensembl reference number ENSG00000166855.9 or a complement thereof. In some embodiments, the CLPX pre-mRNA transcript comprises a sequence with at least about 80%, 85%, 90%, 95%, 96%, 97%, 98%, 99% or 100% sequence identity to a CLPX pre-mRNA transcript or a complement thereof described herein. In some embodiments, the targeted portion of the CLPX pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the CLPX pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a DMTF1 genomic sequence. In some embodiments, the ASO targets a NSAE pre-mRNA transcript from a DMTF1 genomic sequence comprising a NSAE exon. In some embodiments, the ASO targets a NSAE pre-mRNA transcript from a DMTF1 genomic sequence comprising an intron flanking the 3′ splice site of the NSAE exon and an intron flanking the 5′ splice site of a NSAE exon. In some embodiments, the ASO targets a NSAE pre-mRNA transcript of DMTF1. In some embodiments, the ASO targets a DMTF1 pre-mRNA sequence comprising a NSAE exon. In some embodiments, the ASO targets a DMTF1 pre-mRNA sequence comprising an intron flanking the 3′ splice site of the NSAE exon. In some embodiments, the ASO targets a DMTF1 pre-mRNA sequence comprising an intron flanking the 5′ splice site of the NSAE exon.

In some embodiments, the DMTF1 pre-mRNA transcript is encoded by a genetic sequence with at least about 80%, 85%, 90%, 95%, 96%, 97%, 98%, 99% or 100% sequence identity to the Ensembl reference number ENSG00000135164.18 or a complement thereof. In some embodiments, the DMTF1 pre-mRNA transcript comprises a sequence with at least about 80%, 85%, 90%, 95%, 96%, 97%, 98%, 99% or 100% sequence identity to a DMTF1 pre-mRNA transcript or a complement thereof described herein. In some embodiments, the targeted portion of the DMTF1 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the DMTF1 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a DNAJB2 genomic sequence. In some embodiments, the ASO targets a NSAE pre-mRNA transcript from a DNAJB2 genomic sequence comprising a NSAE exon. In some embodiments, the ASO targets a NSAE pre-mRNA transcript from a DNAJB2 genomic sequence comprising an intron flanking the 3′ splice site of the NSAE exon and an intron flanking the 5′ splice site of a NSAE exon. In some embodiments, the ASO targets a NSAE pre-mRNA transcript of DNAJB2. In some embodiments, the ASO targets a DNAJB2 pre-mRNA sequence comprising a NSAE exon. In some embodiments, the ASO targets a DNAJB2 pre-mRNA sequence comprising an intron flanking the 3′ splice site of the NSAE exon. In some embodiments, the ASO targets a DNAJB2 pre-mRNA sequence comprising an intron flanking the 5′ splice site of the NSAE exon.

In some embodiments, the DNAJB2 pre-mRNA transcript is encoded by a genetic sequence with at least about 80%, 85%, 90%, 95%, 96%, 97%, 98%, 99% or 100% sequence identity to the Ensembl reference number ENSG00000135924.15 or a complement thereof. In some embodiments, the DNAJB2 pre-mRNA transcript comprises a sequence with at least about 80%, 85%, 90%, 95%, 96%, 97%, 98%, 99% or 100% sequence identity to a DNAJB2 pre-mRNA transcript or a complement thereof described herein. In some embodiments, the targeted portion of the DNAJB2 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the DNAJB2 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a EIF2AK3 genomic sequence. In some embodiments, the ASO targets a NSAE pre-mRNA transcript from a EIF2AK3 genomic sequence comprising a NSAE exon. In some embodiments, the ASO targets a NSAE pre-mRNA transcript from a EIF2AK3 genomic sequence comprising an intron flanking the 3′ splice site of the NSAE exon and an intron flanking the 5′ splice site of a NSAE exon. In some embodiments, the ASO targets a NSAE pre-mRNA transcript of EIF2AK3. In some embodiments, the ASO targets a EIF2AK3 pre-mRNA sequence comprising a NSAE exon. In some embodiments, the ASO targets a EIF2AK3 pre-mRNA sequence comprising an intron flanking the 3′ splice site of the NSAE exon. In some embodiments, the ASO targets a EIF2AK3 pre-mRNA sequence comprising an intron flanking the 5′ splice site of the NSAE exon.

In some embodiments, the EIF2AK3 pre-mRNA transcript is encoded by a genetic sequence with at least about 80%, 85%, 90%, 95%, 96%, 97%, 98%, 99% or 100% sequence identity to the Ensembl reference number ENSG00000172071.14 or a complement thereof. In some embodiments, the EIF2AK3 pre-mRNA transcript comprises a sequence with at least about 80%, 85%, 90%, 95%, 96%, 97%, 98%, 99% or 100% sequence identity to a EIF2AK3 pre-mRNA transcript or a complement thereof described herein. In some embodiments, the targeted portion of the EIF2AK3 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the EIF2AK3 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a GBA genomic sequence. In some embodiments, the ASO targets a NSAE pre-mRNA transcript from a GBA genomic sequence comprising a NSAE exon. In some embodiments, the ASO targets a NSAE pre-mRNA transcript from a GBA genomic sequence comprising an intron flanking the 3′ splice site of the NSAE exon and an intron flanking the 5′ splice site of a NSAE exon. In some embodiments, the ASO targets a NSAE pre-mRNA transcript of GBA. In some embodiments, the ASO targets a GBA pre-mRNA sequence comprising a NSAE exon. In some embodiments, the ASO targets a GBA pre-mRNA sequence comprising an intron flanking the 3′ splice site of the NSAE exon. In some embodiments, the ASO targets a GBA pre-mRNA sequence comprising an intron flanking the 5′ splice site of the NSAE exon.

In some embodiments, the GBA pre-mRNA transcript is encoded by a genetic sequence with at least about 80%, 85%, 90%, 95%, 96%, 97%, 98%, 99% or 100% sequence identity to the Ensembl reference number ENSG00000177628.16 or ENSG00000262446.5 or a complement thereof. In some embodiments, the GBA pre-mRNA transcript comprises a sequence with at least about 80%, 85%, 90%, 95%, 96%, 97%, 98%, 99% or 100% sequence identity to a GBA pre-mRNA transcript or a complement thereof described herein. In some embodiments, the targeted portion of the GBA pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the GBA pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a GRN genomic sequence. In some embodiments, the ASO targets a NSAE pre-mRNA transcript from a GRN genomic sequence comprising a NSAE exon. In some embodiments, the ASO targets a NSAE pre-mRNA transcript from a GRN genomic sequence comprising an intron flanking the 3′ splice site of the NSAE exon and an intron flanking the 5′ splice site of a NSAE exon. In some embodiments, the ASO targets a NSAE pre-mRNA transcript of GRN. In some embodiments, the ASO targets a GRN pre-mRNA sequence comprising a NSAE exon. In some embodiments, the ASO targets a GRN pre-mRNA sequence comprising an intron flanking the 3′ splice site of the NSAE exon. In some embodiments, the ASO targets a GRN pre-mRNA sequence comprising an intron flanking the 5′ splice site of the NSAE exon.

In some embodiments, the GRN pre-mRNA transcript is encoded by a genetic sequence with at least about 80%, 85%, 90%, 95%, 96%, 97%, 98%, 99% or 100% sequence identity to the Ensembl reference number ENSG00000030582.18 or a complement thereof. In some embodiments, the GRN pre-mRNA transcript comprises a sequence with at least about 80%, 85%, 90%, 95%, 96%, 97%, 98%, 99% or 100% sequence identity to a GRN pre-mRNA transcript or a complement thereof described herein. In some embodiments, the targeted portion of the GRN pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the GRN pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a HSPA9 genomic sequence. In some embodiments, the ASO targets a NSAE pre-mRNA transcript from a HSPA9 genomic sequence comprising a NSAE exon. In some embodiments, the ASO targets a NSAE pre-mRNA transcript from a HSPA9 genomic sequence comprising an intron flanking the 3′ splice site of the NSAE exon and an intron flanking the 5′ splice site of a NSAE exon. In some embodiments, the ASO targets a NSAE pre-mRNA transcript of HSPA9. In some embodiments, the ASO targets a HSPA9 pre-mRNA sequence comprising a NSAE exon. In some embodiments, the ASO targets a HSPA9 pre-mRNA sequence comprising an intron flanking the 3′ splice site of the NSAE exon. In some embodiments, the ASO targets a HSPA9 pre-mRNA sequence comprising an intron flanking the 5′ splice site of the NSAE exon.

In some embodiments, the HSPA9 pre-mRNA transcript is encoded by a genetic sequence with at least about 80%, 85%, 90%, 95%, 96%, 97%, 98%, 99% or 100% sequence identity to the Ensembl reference number ENSG00000113013.15 or a complement thereof. In some embodiments, the HSPA9 pre-mRNA transcript comprises a sequence with at least about 80%, 85%, 90%, 95%, 96%, 97%, 98%, 99% or 100% sequence identity to a HSPA9 pre-mRNA transcript or a complement thereof described herein. In some embodiments, the targeted portion of the HSPA9 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the HSPA9 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a JAG1 genomic sequence. In some embodiments, the ASO targets a NSAE pre-mRNA transcript from a JAG1 genomic sequence comprising a NSAE exon. In some embodiments, the ASO targets a NSAE pre-mRNA transcript from a JAG1 genomic sequence comprising an intron flanking the 3′ splice site of the NSAE exon and an intron flanking the 5′ splice site of a NSAE exon. In some embodiments, the ASO targets a NSAE pre-mRNA transcript of JAG1. In some embodiments, the ASO targets a JAG1 pre-mRNA sequence comprising a NSAE exon. In some embodiments, the ASO targets a JAG1 pre-mRNA sequence comprising an intron flanking the 3′ splice site of the NSAE exon. In some embodiments, the ASO targets a JAG1 pre-mRNA sequence comprising an intron flanking the 5′ splice site of the NSAE exon.

In some embodiments, the JAG1 pre-mRNA transcript is encoded by a genetic sequence with at least about 80%, 85%, 90%, 95%, 96%, 97%, 98%, 99% or 100% sequence identity to the Ensembl reference number ENSG00000101384.12 or a complement thereof. In some embodiments, the JAG1 pre-mRNA transcript comprises a sequence with at least about 80%, 85%, 90%, 95%, 96%, 97%, 98%, 99% or 100% sequence identity to a JAG1 pre-mRNA transcript or a complement thereof described herein. In some embodiments, the targeted portion of the JAG1 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the JAG1 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a KCNQ2 genomic sequence. In some embodiments, the ASO targets a NSAE pre-mRNA transcript from a KCNQ2 genomic sequence comprising a NSAE exon. In some embodiments, the ASO targets a NSAE pre-mRNA transcript from a KCNQ2 genomic sequence comprising an intron flanking the 3′ splice site of the NSAE exon and an intron flanking the 5′ splice site of a NSAE exon. In some embodiments, the ASO targets a NSAE pre-mRNA transcript of KCNQ2. In some embodiments, the ASO targets a KCNQ2 pre-mRNA sequence comprising a NSAE exon. In some embodiments, the ASO targets a KCNQ2 pre-mRNA sequence comprising an intron flanking the 3′ splice site of the NSAE exon. In some embodiments, the ASO targets a KCNQ2 pre-mRNA sequence comprising an intron flanking the 5′ splice site of the NSAE exon.

In some embodiments, the KCNQ2 pre-mRNA transcript is encoded by a genetic sequence with at least about 80%, 85%, 90%, 95%, 96%, 97%, 98%, 99% or 100% sequence identity to the Ensembl reference number ENSG00000075043.18 or ENSG00000281151.2 or a complement thereof. In some embodiments, the KCNQ2 pre-mRNA transcript comprises a sequence with at least about 80%, 85%, 90%, 95%, 96%, 97%, 98%, 99% or 100% sequence identity to a KCNQ2 pre-mRNA transcript or a complement thereof described herein. In some embodiments, the targeted portion of the KCNQ2 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the KCNQ2 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a KL genomic sequence. In some embodiments, the ASO targets a NSAE pre-mRNA transcript from a KL genomic sequence comprising a NSAE exon. In some embodiments, the ASO targets a NSAE pre-mRNA transcript from a KL genomic sequence comprising an intron flanking the 3′ splice site of the NSAE exon and an intron flanking the 5′ splice site of a NSAE exon. In some embodiments, the ASO targets a NSAE pre-mRNA transcript of KL. In some embodiments, the ASO targets a KL pre-mRNA sequence comprising a NSAE exon. In some embodiments, the ASO targets a KL pre-mRNA sequence comprising an intron flanking the 3′ splice site of the NSAE exon. In some embodiments, the ASO targets a KL pre-mRNA sequence comprising an intron flanking the 5′ splice site of the NSAE exon.

In some embodiments, the KL pre-mRNA transcript is encoded by a genetic sequence with at least about 80%, 85%, 90%, 95%, 96%, 97%, 98%, 99% or 100% sequence identity to the Ensembl reference number ENSG00000133116.8 or a complement thereof. In some embodiments, the KL pre-mRNA transcript comprises a sequence with at least about 80%, 85%, 90%, 95%, 96%, 97%, 98%, 99% or 100% sequence identity to a KL pre-mRNA transcript or a complement thereof described herein. In some embodiments, the targeted portion of the KL pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the KL pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a OPTN genomic sequence. In some embodiments, the ASO targets a NSAE pre-mRNA transcript from a OPTN genomic sequence comprising a NSAE exon. In some embodiments, the ASO targets a NSAE pre-mRNA transcript from a OPTN genomic sequence comprising an intron flanking the 3′ splice site of the NSAE exon and an intron flanking the 5′ splice site of a NSAE exon. In some embodiments, the ASO targets a NSAE pre-mRNA transcript of OPTN. In some embodiments, the ASO targets a OPTN pre-mRNA sequence comprising a NSAE exon. In some embodiments, the ASO targets a OPTN pre-mRNA sequence comprising an intron flanking the 3′ splice site of the NSAE exon. In some embodiments, the ASO targets a OPTN pre-mRNA sequence comprising an intron flanking the 5′ splice site of the NSAE exon.

In some embodiments, the OPTN pre-mRNA transcript is encoded by a genetic sequence with at least about 80%, 85%, 90%, 95%, 96%, 97%, 98%, 99% or 100% sequence identity to the Ensembl reference number ENSG00000123240.17 or a complement thereof. In some embodiments, the OPTN pre-mRNA transcript comprises a sequence with at least about 80%, 85%, 90%, 95%, 96%, 97%, 98%, 99% or 100% sequence identity to a OPTN pre-mRNA transcript or a complement thereof described herein. In some embodiments, the targeted portion of the OPTN pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the OPTN pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a RBL1 genomic sequence. In some embodiments, the ASO targets a NSAE pre-mRNA transcript from a RBL1 genomic sequence comprising a NSAE exon. In some embodiments, the ASO targets a NSAE pre-mRNA transcript from a RBL1 genomic sequence comprising an intron flanking the 3′ splice site of the NSAE exon and an intron flanking the 5′ splice site of a NSAE exon. In some embodiments, the ASO targets a NSAE pre-mRNA transcript of RBL1. In some embodiments, the ASO targets a RBL1 pre-mRNA sequence comprising a NSAE exon. In some embodiments, the ASO targets a RBL1 pre-mRNA sequence comprising an intron flanking the 3′ splice site of the NSAE exon. In some embodiments, the ASO targets a RBL1 pre-mRNA sequence comprising an intron flanking the 5′ splice site of the NSAE exon.

In some embodiments, the RBL1 pre-mRNA transcript is encoded by a genetic sequence with at least about 80%, 85%, 90%, 95%, 96%, 97%, 98%, 99% or 100% sequence identity to the Ensembl reference number ENSG00000080839.12 or a complement thereof. In some embodiments, the RBL1 pre-mRNA transcript comprises a sequence with at least about 80%, 85%, 90%, 95%, 96%, 97%, 98%, 99% or 100% sequence identity to a RBL1 pre-mRNA transcript or a complement thereof described herein. In some embodiments, the targeted portion of the RBL1 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the RBL1 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a SEMA4A genomic sequence. In some embodiments, the ASO targets a NSAE pre-mRNA transcript from a SEMA4A genomic sequence comprising a NSAE exon. In some embodiments, the ASO targets a NSAE pre-mRNA transcript from a SEMA4A genomic sequence comprising an intron flanking the 3′ splice site of the NSAE exon and an intron flanking the 5′ splice site of a NSAE exon. In some embodiments, the ASO targets a NSAE pre-mRNA transcript of SEMA4A. In some embodiments, the ASO targets a SEMA4A pre-mRNA sequence comprising a NSAE exon. In some embodiments, the ASO targets a SEMA4A pre-mRNA sequence comprising an intron flanking the 3′ splice site of the NSAE exon. In some embodiments, the ASO targets a SEMA4A pre-mRNA sequence comprising an intron flanking the 5′ splice site of the NSAE exon.

In some embodiments, the SEMA4A pre-mRNA transcript is encoded by a genetic sequence with at least about 80%, 85%, 90%, 95%, 96%, 97%, 98%, 99% or 100% sequence identity to the Ensembl reference number ENSG00000196189.13 or a complement thereof. In some embodiments, the SEMA4A pre-mRNA transcript comprises a sequence with at least about 80%, 85%, 90%, 95%, 96%, 97%, 98%, 99% or 100% sequence identity to a SEMA4A pre-mRNA transcript or a complement thereof described herein. In some embodiments, the targeted portion of the SEMA4A pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the SEMA4A pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a SHANK3 genomic sequence. In some embodiments, the ASO targets a NSAE pre-mRNA transcript from a SHANK3 genomic sequence comprising a NSAE exon. In some embodiments, the ASO targets a NSAE pre-mRNA transcript from a SHANK3 genomic sequence comprising an intron flanking the 3′ splice site of the NSAE exon and an intron flanking the 5′ splice site of a NSAE exon. In some embodiments, the ASO targets a NSAE pre-mRNA transcript of SHANK3. In some embodiments, the ASO targets a SHANK3 pre-mRNA sequence comprising a NSAE exon. In some embodiments, the ASO targets a SHANK3 pre-mRNA sequence comprising an intron flanking the 3′ splice site of the NSAE exon. In some embodiments, the ASO targets a SHANK3 pre-mRNA sequence comprising an intron flanking the 5′ splice site of the NSAE exon.

In some embodiments, the SHANK3 pre-mRNA transcript is encoded by a genetic sequence with at least about 80%, 85%, 90%, 95%, 96%, 97%, 98%, 99% or 100% sequence identity to the Ensembl reference number ENSG00000251322.9 or a complement thereof. In some embodiments, the SHANK3 pre-mRNA transcript comprises a sequence with at least about 80%, 85%, 90%, 95%, 96%, 97%, 98%, 99% or 100% sequence identity to a SHANK3 pre-mRNA transcript or a complement thereof described herein. In some embodiments, the targeted portion of the SHANK3 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the SHANK3 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a SIRT2 genomic sequence. In some embodiments, the ASO targets a NSAE pre-mRNA transcript from a SIRT2 genomic sequence comprising a NSAE exon. In some embodiments, the ASO targets a NSAE pre-mRNA transcript from a SIRT2 genomic sequence comprising an intron flanking the 3′ splice site of the NSAE exon and an intron flanking the 5′ splice site of a NSAE exon. In some embodiments, the ASO targets a NSAE pre-mRNA transcript of SIRT2. In some embodiments, the ASO targets a SIRT2 pre-mRNA sequence comprising a NSAE exon. In some embodiments, the ASO targets a SIRT2 pre-mRNA sequence comprising an intron flanking the 3′ splice site of the NSAE exon. In some embodiments, the ASO targets a SIRT2 pre-mRNA sequence comprising an intron flanking the 5′ splice site of the NSAE exon.

In some embodiments, the SIRT2 pre-mRNA transcript is encoded by a genetic sequence with at least about 80%, 85%, 90%, 95%, 96%, 97%, 98%, 99% or 100% sequence identity to the Ensembl reference number ENSG00000068903.20 or ENSG00000283100.2 or a complement thereof. In some embodiments, the SIRT2 pre-mRNA transcript comprises a sequence with at least about 80%, 85%, 90%, 95%, 96%, 97%, 98%, 99% or 100% sequence identity to a SIRT2 pre-mRNA transcript or a complement thereof described herein. In some embodiments, the targeted portion of the SIRT2 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the SIRT2 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a SLC4A11 genomic sequence. In some embodiments, the ASO targets a NSAE pre-mRNA transcript from a SLC4A11 genomic sequence comprising a NSAE exon. In some embodiments, the ASO targets a NSAE pre-mRNA transcript from a SLC4A11 genomic sequence comprising an intron flanking the 3′ splice site of the NSAE exon and an intron flanking the 5′ splice site of a NSAE exon. In some embodiments, the ASO targets a NSAE pre-mRNA transcript of SLC4A11. In some embodiments, the ASO targets a SLC4A11 pre-mRNA sequence comprising a NSAE exon. In some embodiments, the ASO targets a SLC4A11 pre-mRNA sequence comprising an intron flanking the 3′ splice site of the NSAE exon. In some embodiments, the ASO targets a SLC4A11 pre-mRNA sequence comprising an intron flanking the 5′ splice site of the NSAE exon.

In some embodiments, the SLC4A11 pre-mRNA transcript is encoded by a genetic sequence with at least about 80%, 85%, 90%, 95%, 96%, 97%, 98%, 99% or 100% sequence identity to the Ensembl reference number ENSG00000088836.14 or a complement thereof. In some embodiments, the SLC4A11 pre-mRNA transcript comprises a sequence with at least about 80%, 85%, 90%, 95%, 96%, 97%, 98%, 99% or 100% sequence identity to a SLC4A1l pre-mRNA transcript or a complement thereof described herein. In some embodiments, the targeted portion of the SLC4A11 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the SLC4A11 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a STAT3 genomic sequence. In some embodiments, the ASO targets a NSAE pre-mRNA transcript from a STAT3 genomic sequence comprising a NSAE exon. In some embodiments, the ASO targets a NSAE pre-mRNA transcript from a STAT3 genomic sequence comprising an intron flanking the 3′ splice site of the NSAE exon and an intron flanking the 5′ splice site of a NSAE exon. In some embodiments, the ASO targets a NSAE pre-mRNA transcript of STAT3. In some embodiments, the ASO targets a STAT3 pre-mRNA sequence comprising a NSAE exon. In some embodiments, the ASO targets a STAT3 pre-mRNA sequence comprising an intron flanking the 3′ splice site of the NSAE exon. In some embodiments, the ASO targets a STAT3 pre-mRNA sequence comprising an intron flanking the 5′ splice site of the NSAE exon.

In some embodiments, the STAT3 pre-mRNA transcript is encoded by a genetic sequence with at least about 80%, 85%, 90%, 95%, 96%, 97%, 98%, 99% or 100% sequence identity to the Ensembl reference number ENSG00000168610.14 or a complement thereof. In some embodiments, the STAT3 pre-mRNA transcript comprises a sequence with at least about 80%, 85%, 90%, 95%, 96%, 97%, 98%, 99% or 100% sequence identity to a STAT3 pre-mRNA transcript or a complement thereof described herein. In some embodiments, the targeted portion of the STAT3 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the STAT3 pre-mRNA comprises the exemplary NMD-inducing Alternative 5′ splice site or 3′ splice site as indicated in Table 5C.

In some embodiments, the ASOs disclosed herein target a NSAE pre-mRNA transcribed from a TP53BP2 genomic sequence. In some embodiments, the ASO targets a NSAE pre-mRNA transcript from a TP53BP2 genomic sequence comprising a NSAE exon. In some embodiments, the ASO targets a NSAE pre-mRNA transcript from a TP53BP2 genomic sequence comprising an intron flanking the 3′ splice site of the NSAE exon and an intron flanking the 5′ splice site of a NSAE exon. In some embodiments, the ASO targets a NSAE pre-mRNA transcript of TP53BP2. In some embodiments, the ASO targets a TP53BP2 pre-mRNA sequence comprising a NSAE exon. In some embodiments, the ASO targets a TP53BP2 pre-mRNA sequence comprising an intron flanking the 3′ splice site of the NSAE exon. In some embodiments, the ASO targets a TP53BP2 pre-mRNA sequence comprising an intron flanking the 5′ splice site of the NSAE exon.

In some embodiments, the TP53BP2 pre-mRNA transcript is encoded by a genetic sequence with at least about 80%, 85%, 90%, 95%, 96%, 97%, 98%, 99% or 100% sequence identity to the Ensembl reference number ENSG00000143514.17 or a complement thereof. In some embodiments, the TP53BP2 pre-mRNA transcript comprises a sequence with at least about 80%, 85%, 90%, 95%, 96%, 97%, 98%, 99% or 100% sequence identity to a TP53BP2 pre-mRNA transcript or a complement thereof described herein. In some embodiments, the targeted portion of the TP53BP2 pre-mRNA comprises a sequence with at least 80%, 85%, 90%, 95%, 97%, or 100% sequence identity to a region comprising at least 8 contiguous nucleic acids of a sequence of Table 6A or Table 6B or complements thereof. In some embodiments, the target portion of the TP53BP2 pre-mRNA comprises the exemplary NMD-inducing Alternative 5′ splice site or 3′ splice site as indicated in Table 5C.

TABLE 6A
(Exemplary ASO sequences)

| Target | ASO sequences (SEQ ID NOs) |
|---|---|
| ABCC3 | SEQ ID NOs: 1301-1328 |
| ABCC5 | SEQ ID NOs: 1329-1368 |
| ABCC8 | SEQ ID NOs: 1369-1401 |
| ACOX2 | SEQ ID NOs: 1402-1436 |
| ACSF3 | SEQ ID NOs: 1437-1464 |
| ADAM17 | SEQ ID NOs: 1465-1485 |
| ADAMTS13 | SEQ ID NOs: 1486-1520 |
| ALAD | SEQ ID NOs: 1521-1571 |
| ANKS3 | SEQ ID NOs: 1572-1603 |
| ANO4 | SEQ ID NOs: 1604-1633 |
| AP3B2 | SEQ ID NOs: 1634-1663 |
| AP5Z1 | SEQ ID NOs: 1664-1688 |
| ARNTL | SEQ ID NOs: 1689-1724 |
| BBS2 | SEQ ID NOs: 1725-1766 |
| BRD9 | SEQ ID NOs: 1767-1807 |
| CALM3 | SEQ ID NOs: 1808-1913 |
| CARS1 | SEQ ID NOs: 1914-1940 |
| CASP9 | SEQ ID NOs: 1941-1969 |
| CD58 | SEQ ID NOs: 1970-1993 |
| CHFR | SEQ ID NOs: 1994-2031 |
| CLCN2 | SEQ ID NOs: 2032-2099 |
| CNOT3 | SEQ ID NOs: 2100-2128 |
| CNTROB | SEQ ID NOs: 2129-2171 |
| CTF1 | SEQ ID NOs: 2172-2250 |
| CYP3A5 | SEQ ID NOs: 2251-2292 |
| DHDDS | SEQ ID NOs: 2293-2314 |
| DNHD1 | SEQ ID NOs: 2315-2358 |
| ENTPD4 | SEQ ID NOs: 2359-2416 |
| ESRRA | SEQ ID NOs: 2417-2453 |
| ESS2 | SEQ ID NOs: 2454-2503 |
| ETAA1 | SEQ ID NOs: 2504-2568 |
| FASTKD3 | SEQ ID NOs: 2569-2610 |
| FREM1 | SEQ ID NOs: 2611-2638 |
| GAS8 | SEQ ID NOs: 2639-2677 |
| GGA3 | SEQ ID NOs: 2678-2713 |
| GGT1 | SEQ ID NOs: 2714-2735 |
| GLMN | SEQ ID NOs: 2736-2792 |
| GRB14 | SEQ ID NOs: 2793-2820 |
| HDAC3 | SEQ ID NOs: 2821-2842 |
| HP1BP3 | SEQ ID NOs: 2843-2878 |
| HPS4 | SEQ ID NOs: 2879-2917 |
| IDUA | SEQ ID NOs: 2918-2934 |
| IFT122 | SEQ ID NOs: 2935-2969 |
| IMPDH1 | SEQ ID NOs: 2970-2991 |
| KCTD3 | SEQ ID NOs: 2992-3023 |
| KYAT1 | SEQ ID NOs: 3024-3047 |
| MAP3K7 | SEQ ID NOs: 3048-3068 |
| NBEAL2 | SEQ ID NOs: 3069-3094 |
| NOP58 | SEQ ID NOs: 3095-3132 |
| NPHP1 | SEQ ID NOs: 3133-3227 |
| NUP188 | SEQ ID NOs: 3228-3271 |
| PHKA2 | SEQ ID NOs: 3272-3299 |
| PIDD1 | SEQ ID NOs: 3300-3378 |
| PKD1 | SEQ ID NOs: 3379-3433 |
| PLA2G6 | SEQ ID NOs: 3434-3484 |
| PLD2 | SEQ ID NOs: 3485-3545 |
| PLEKHG5 | SEQ ID NOs: 3546-3590 |
| PRMT7 | SEQ ID NOs: 3591-3627 |
| PRODH | SEQ ID NOs: 3628-3642 |
| PRPF3 | SEQ ID NOs: 3643-3670 |
| PRPF4 | SEQ ID NOs: 3671-3695 |
| RAD52 | SEQ ID NOs: 3696-3720 |
| REXO1 | SEQ ID NOs: 3721-3798 |
| RFX5 | SEQ ID NOs: 3799-3829 |
| RUFY3 | SEQ ID NOs: 3830-3850 |
| SEMA3B | SEQ ID NOs: 3851-3892 |
| SEMA3F | SEQ ID NOs: 3893-3917 |
| SH2D3A | SEQ ID NOs: 3918-3939 |
| SIRT3 | SEQ ID NOs: 3940-3987 |
| SKIV2L | SEQ ID NOs: 3988-4010 |
| SLC25A13 | SEQ ID NOs: 4011-4032 |
| SLC2A13 | SEQ ID NOs: 4033-4100 |
| SLC30A10 | SEQ ID NOs: 4101-4125 |
| SLC30A9 | SEQ ID NOs: 4126-4155 |
| SMTN | SEQ ID NOs: 4156-4184 |
| STXBP2 | SEQ ID NOs: 4185-4299 |
| SYNGAP1 | SEQ ID NOs: 4300-4352 |
| TBL2 | SEQ ID NOs: 4353-4428 |
| TECPR2 | SEQ ID NOs: 4429-4466 |
| TNK2 | SEQ ID NOs: 4467-4518 |
| TOE1 | SEQ ID NOs: 4519-4552 |
| TRMT2A | SEQ ID NOs: 4553-4591 |
| TRPV4 | SEQ ID NOs: 4592-4614 |
| UROD | SEQ ID NOs: 4615-4641 |
| WDR62 | SEQ ID NOs: 4642-4668 |
| WRAP53 | SEQ ID NOs: 4669-4692 |
| XPO1 | SEQ ID NOs: 4693-4711 |

In some embodiments, an ASO that targets a pre-mRNA disclosed herein is selected from the group consisting of SEQ ID NOs: 1301-4711.

In some embodiments, an ASO that targets a ABCC3 pre-mRNA is selected from the group consisting of SEQ ID NOs: 1301-1328. In some embodiments, an ASO that targets a ABCC5 pre-mRNA is selected from the group consisting of SEQ ID NOs: 1329-1368. In some embodiments, an ASO that targets a ABCC8 pre-mRNA is selected from the group consisting of SEQ ID NOs: 1369-1401. In some embodiments, an ASO that targets a ACOX2 pre-mRNA is selected from the group consisting of SEQ ID NOs: 1402-1436. In some embodiments, an ASO that targets a ACSF3 pre-mRNA is selected from the group consisting of SEQ ID NOs: 1437-1464. In some embodiments, an ASO that targets a ADAM17 pre-mRNA is selected from the group consisting of SEQ ID NOs: 1465-1485. In some embodiments, an ASO that targets a ADAMTS13 pre-mRNA is selected from the group consisting of SEQ ID NOs: 1486-1520. In some embodiments, an ASO that targets a ALAD pre-mRNA is selected from the group consisting of SEQ ID NOs: 1521-1571. In some embodiments, an ASO that targets a ANKS3 pre-mRNA is selected from the group consisting of SEQ ID NOs: 1572-1603. In some embodiments, an ASO that targets a ANO4 pre-mRNA is selected from the group consisting of SEQ ID NOs: 1604-1633. In some embodiments, an ASO that targets a AP3B2 pre-mRNA is selected from the group consisting of SEQ ID NOs: 1634-1663. In some embodiments, an ASO that targets a AP5Z1 pre-mRNA is selected from the group consisting of SEQ ID NOs: 1664-1688. In some embodiments, an ASO that targets a ARNTL pre-mRNA is selected from the group consisting of SEQ ID NOs: 1689-1724. In some embodiments, an ASO that targets a BBS2 pre-mRNA is selected from the group consisting of SEQ ID NOs: 1725-1766. In some embodiments, an ASO that targets a BRD9 pre-mRNA is selected from the group consisting of SEQ ID NOs: 1767-1807. In some embodiments, an ASO that targets a CALM3 pre-mRNA is selected from the group consisting of SEQ ID NOs: 1808-1913. In some embodiments, an ASO that targets a CARS1 pre-mRNA is selected from the group consisting of SEQ ID NOs: 1914-1940. In some embodiments, an ASO that targets a CASP9 pre-mRNA is selected from the group consisting of SEQ ID NOs: 1941-1969. In some embodiments, an ASO that targets a CD58 pre-mRNA is selected from the group consisting of SEQ ID NOs: 1970-1993. In some embodiments, an ASO that targets a CIHFR pre-mRNA is selected from the group consisting of SEQ ID NOs: 1994-2031. In some embodiments, an ASO that targets a CLCN2 pre-mRNA is selected from the group consisting of SEQ ID NOs: 2032-2099. In some embodiments, an ASO that targets a CNOT3 pre-mRNA is selected from the group consisting of SEQ ID NOs: 2100-2128. In some embodiments, an ASO that targets a CNTROB pre-mRNA is selected from the group consisting of SEQ ID NOs: 2129-2171. In some embodiments, an ASO that targets a CTF1 pre-mRNA is selected from the group consisting of SEQ ID NOs: 2172-2250. In some embodiments, an ASO that targets a CYP3A5 pre-mRNA is selected from the group consisting of SEQ ID NOs: 2251-2292. In some embodiments, an ASO that targets a DHDDS pre-mRNA is selected from the group consisting of SEQ ID NOs: 2293-2314. In some embodiments, an ASO that targets a DNHD1 pre-mRNA is selected from the group consisting of SEQ ID NOs: 2315-2358. In some embodiments, an ASO that targets a ENTPD4 pre-mRNA is selected from the group consisting of SEQ ID NOs: 2359-2416. In some embodiments, an ASO that targets a ESRRA pre-mRNA is selected from the group consisting of SEQ ID NOs: 2417-2453. In some embodiments, an ASO that targets a ESS2 pre-mRNA is selected from the group consisting of SEQ ID NOs: 2454-2503. In some embodiments, an ASO that targets a ETAA1 pre-mRNA is selected from the group consisting of SEQ ID NOs: 2504-2568. In some embodiments, an ASO that targets a FASTKD3 pre-mRNA is selected from the group consisting of SEQ ID NOs: 2569-2610. In some embodiments, an ASO that targets a FREM1 pre-mRNA is selected from the group consisting of SEQ ID NOs: 2611-2638. In some embodiments, an ASO that targets a GAS8 pre-mRNA is selected from the group consisting of SEQ ID NOs: 2639-2677. In some embodiments, an ASO that targets a GGA3 pre-mRNA is selected from the group consisting of SEQ ID NOs: 2678-2713. In some embodiments, an ASO that targets a GGT1 pre-mRNA is selected from the group consisting of SEQ ID NOs: 2714-2735. In some embodiments, an ASO that targets a GLMN pre-mRNA is selected from the group consisting of SEQ ID NOs: 2736-2792. In some embodiments, an ASO that targets a GRB14 pre-mRNA is selected from the group consisting of SEQ ID NOs: 2793-2820. In some embodiments, an ASO that targets a HDAC3 pre-mRNA is selected from the group consisting of SEQ ID NOs: 2821-2842. In some embodiments, an ASO that targets a HP1BP3 pre-mRNA is selected from the group consisting of SEQ ID NOs: 2843-2878. In some embodiments, an ASO that targets a HPS4 pre-mRNA is selected from the group consisting of SEQ ID NOs: 2879-2917. In some embodiments, an ASO that targets a IDUA pre-mRNA is selected from the group consisting of SEQ ID NOs: 2918-2934. In some embodiments, an ASO that targets a IFT122 pre-mRNA is selected from the group consisting of SEQ ID NOs: 2935-2969. In some embodiments, an ASO that targets a IMPDH1 pre-mRNA is selected from the group consisting of SEQ ID NOs: 2970-2991. In some embodiments, an ASO that targets a KCTD3 pre-mRNA is selected from the group consisting of SEQ ID NOs: 2992-3023. In some embodiments, an ASO that targets a KYAT1 pre-mRNA is selected from the group consisting of SEQ ID NOs: 3024-3047. In some embodiments, an ASO that targets a MAP3K7 pre-mRNA is selected from the group consisting of SEQ ID NOs: 3048-3068. In some embodiments, an ASO that targets a NBEAL2 pre-mRNA is selected from the group consisting of SEQ ID NOs: 3069-3094. In some embodiments, an ASO that targets a NOP58 pre-mRNA is selected from the group consisting of SEQ ID NOs: 3095-3132. In some embodiments, an ASO that targets a NPHP1 pre-mRNA is selected from the group consisting of SEQ ID NOs: 3133-3227. In some embodiments, an ASO that targets a NUP188 pre-mRNA is selected from the group consisting of SEQ ID NOs: 3228-3271. In some embodiments, an ASO that targets a PHKA2 pre-mRNA is selected from the group consisting of SEQ ID NOs: 3272-3299. In some embodiments, an ASO that targets a PIDD1 pre-mRNA is selected from the group consisting of SEQ ID NOs: 3300-3378. In some embodiments, an ASO that targets a PKD1 pre-mRNA is selected from the group consisting of SEQ ID NOs: 3379-3433. In some embodiments, an ASO that targets a PLA2G6 pre-mRNA is selected from the group consisting of SEQ ID NOs: 3434-3484. In some embodiments, an ASO that targets a PLD2 pre-mRNA is selected from the group consisting of SEQ ID NOs: 3485-3545. In some embodiments, an ASO that targets a PLEKHG5 pre-mRNA is selected from the group consisting of SEQ ID NOs: 3546-3590. In some embodiments, an ASO that targets a PRMT7 pre-mRNA is selected from the group consisting of SEQ ID NOs: 3591-3627. In some embodiments, an ASO that targets a PRODH pre-mRNA is selected from the group consisting of SEQ ID NOs: 3628-3642. In some embodiments, an ASO that targets a PRPF3 pre-mRNA is selected from the group consisting of SEQ ID NOs: 3643-3670. In some embodiments, an ASO that targets a PRPF4 pre-mRNA is selected from the group consisting of SEQ ID NOs: 3671-3695. In some embodiments, an ASO that targets a RAD52 pre-mRNA is selected from the group consisting of SEQ ID NOs: 3696-3720. In some embodiments, an ASO that targets a REXO1 pre-mRNA is selected from the group consisting of SEQ ID NOs: 3721-3798. In some embodiments, an ASO that targets a RFX5 pre-mRNA is selected from the group consisting of SEQ ID NOs: 3799-3829. In some embodiments, an ASO that targets a RUFY3 pre-mRNA is selected from the group consisting of SEQ ID NOs: 3830-3850. In some embodiments, an ASO that targets a SEMA3B pre-mRNA is selected from the group consisting of SEQ ID NOs: 3851-3892. In some embodiments, an ASO that targets a SEMA3F pre-mRNA is selected from the group consisting of SEQ ID NOs: 3893-3917. In some embodiments, an ASO that targets a SH2D3A pre-mRNA is selected from the group consisting of SEQ ID NOs: 3918-3939. In some embodiments, an ASO that targets a SIRT3 pre-mRNA is selected from the group consisting of SEQ ID NOs: 3940-3987. In some embodiments, an ASO that targets a SKIV2L pre-mRNA is selected from the group consisting of SEQ ID NOs: 3988-4010. In some embodiments, an ASO that targets a SLC25A13 pre-mRNA is selected from the group consisting of SEQ ID NOs: 4011-4032. In some embodiments, an ASO that targets a SLC2A13 pre-mRNA is selected from the group consisting of SEQ ID NOs: 4033-4100. In some embodiments, an ASO that targets a SLC30A10 pre-mRNA is selected from the group consisting of SEQ ID NOs: 4101-4125. In some embodiments, an ASO that targets a SLC30A9 pre-mRNA is selected from the group consisting of SEQ ID NOs: 4126-4155. In some embodiments, an ASO that targets a SMTN pre-mRNA is selected from the group consisting of SEQ ID NOs: 4156-4184. In some embodiments, an ASO that targets a STXBP2 pre-mRNA is selected from the group consisting of SEQ ID NOs: 4185-4299. In some embodiments, an ASO that targets a SYNGAP1 pre-mRNA is selected from the group consisting of SEQ ID NOs: 4300-4352. In some embodiments, an ASO that targets a SYNGAP1 pre-mRNA is selected from the group consisting of SEQ ID NOs: 1-165. In some embodiments, an ASO that targets a TBL2 pre-mRNA is selected from the group consisting of SEQ ID NOs: 4353-4428. In some embodiments, an ASO that targets a TECPR2 pre-mRNA is selected from the group consisting of SEQ ID NOs: 4429-4466. In some embodiments, an ASO that targets a TNK2 pre-mRNA is selected from the group consisting of SEQ ID NOs: 4467-4518. In some embodiments, an ASO that targets a TOE1 pre-mRNA is selected from the group consisting of SEQ ID NOs: 4519-4552. In some embodiments, an ASO that targets a TRMT2A pre-mRNA is selected from the group consisting of SEQ ID NOs: 4553-4591. In some embodiments, an ASO that targets a TRPV4 pre-mRNA is selected from the group consisting of SEQ ID NOs: 4592-4614. In some embodiments, an ASO that targets a UROD pre-mRNA is selected from the group consisting of SEQ ID NOs: 4615-4641. In some embodiments, an ASO that targets a WDR62 pre-mRNA is selected from the group consisting of SEQ ID NOs: 4642-4668. In some embodiments, an ASO that targets a WRAP53 pre-mRNA is selected from the group consisting of SEQ ID NOs: 4669-4692. In some embodiments, an ASO that targets a XPO1 pre-mRNA is selected from the group consisting of SEQ ID NOs: 4693-4711.

TABLE 6B
(Exemplary ASO sequences)

| Target | ASO sequences (SEQ ID NOs) |
|---|---|
| ABCA5 | SEQ ID NOs: 4712-4748 |
| ABCA7 | SEQ ID NOs: 4749-4775 |
| ABCD1 | SEQ ID NOs: 4776-4874 |
| ABR | SEQ ID NOs: 4875-4971 |
| ACAD9 | SEQ ID NOs: 4972-5026 |
| ACAP1 | SEQ ID NOs: 5027-5084 |
| ACTN4 | SEQ ID NOs: 5085-5174 |
| ADAMTS13 | SEQ ID NOs: 5175-5209 |
| AKR1E2 | SEQ ID NOs: 5210-5242 |
| ALG3 | SEQ ID NOs: 5243-5411 |
| ANKRD29 | SEQ ID NOs: 5412-5458 |
| AP3M1 | SEQ ID NOs: 5459-5482 |
| ASAP3 | SEQ ID NOs: 5483-5527 |
| ATRX | SEQ ID NOs: 5528-5574 |
| BBS4 | SEQ ID NOs: 5575-5656 |
| CATSPER1 | SEQ ID NOs: 5657-5683 |
| CCDC40 | SEQ ID NOs: 5684-5720 |
| CCDC88B | SEQ ID NOs: 5721-5765 |
| CD4 | SEQ ID NOs: 5766-5806 |
| CES2 | SEQ ID NOs: 5807-5878 |
| COL5A3 | SEQ ID NOs: 5879-5924 |
| CPSF1 | SEQ ID NOs: 5925-5954 |
| CSPP1 | SEQ ID NOs: 5955-5982 |
| CTH | SEQ ID NOs: 5983-6016 |
| DAB2IP | SEQ ID NOs: 6017-6063 |
| DBN1 | SEQ ID NOs: 6064-6105 |
| DDX51 | SEQ ID NOs: 6106-6152 |
| DEAF1 | SEQ ID NOs: 6153-6187 |
| DGKE | SEQ ID NOs: 6188-6214 |
| DMKN | SEQ ID NOs: 6215-6257 |
| DOCK8 | SEQ ID NOs: 6258-6354 |
| DOK5 | SEQ ID NOs: 6355-6408 |
| EIF2B5 | SEQ ID NOs: 6409-6440 |
| ELAC2 | SEQ ID NOs: 6441-6487 |
| ELP1 | SEQ ID NOs: 6488-6519 |
| ETHE1 | SEQ ID NOs: 6520-6570 |
| FANCA | SEQ ID NOs: 6571-6647 |
| FASTK | SEQ ID NOs: 6648-6762 |
| FPGS | SEQ ID NOs: 6763-6799 |
| GAS8 | SEQ ID NOs: 6800-6857 |
| GCK | SEQ ID NOs: 6858-6954 |
| GGA1 | SEQ ID NOs: 6955-7024 |
| GON4L | SEQ ID NOs: 7025-7051 |
| HELQ | SEQ ID NOs: 7052-7096 |
| HPS1 | SEQ ID NOs: 7097-7124 |
| IL17RC | SEQ ID NOs: 7125-7162 |
| INVS | SEQ ID NOs: 7163-7260 |
| IRF3 | SEQ ID NOs: 7261-7379 |
| KLKB1 | SEQ ID NOs: 7380-7405 |
| LAMC3 | SEQ ID NOs: 7406-7454 |
| LDAH | SEQ ID NOs: 7455-7487 |
| LIMS2 | SEQ ID NOs: 7488-7585 |
| MALT1 | SEQ ID NOs: 7586-7641 |
| MAPK13 | SEQ ID NOs: 7642-7751 |
| MCAT | SEQ ID NOs: 7752-7777 |
| MCEE | SEQ ID NOs: 7778-7889 |
| MPI | SEQ ID NOs: 7890-7931 |
| MSTO1 | SEQ ID NOs: 7932-7963 |
| NLE1 | SEQ ID NOs: 7964-7988 |
| NLRC5 | SEQ ID NOs: 7989-8013 |
| NOM1 | SEQ ID NOs: 8014-8048 |
| NPR1 | SEQ ID NOs: 8049-8139 |
| OSGEP | SEQ ID NOs: 8140-8165 |
| PABPC4 | SEQ ID NOs: 8166-8212 |
| PCOLCE | SEQ ID NOs: 8213-8288 |
| PLD2 | SEQ ID NOs: 8289-8305 |
| PNPO | SEQ ID NOs: 8306-8372 |
| POLE | SEQ ID NOs: 8373-8401 |
| PON2 | SEQ ID NOs: 8402-8434 |
| PYCR1 | SEQ ID NOs: 8435-8493 |
| RAD52 | SEQ ID NOs: 8494-8546 |
| RHBDF2 | SEQ ID NOs: 8547-8579 |
| RMND1 | SEQ ID NOs: 8580-8604 |
| ROBO3 | SEQ ID NOs: 8605-8756 |
| RPGRIP1 | SEQ ID NOs: 8757-8821 |
| RTTN | SEQ ID NOs: 8822-8855 |
| SIK3 | SEQ ID NOs: 8856-8912 |
| SLC12A7 | SEQ ID NOs: 8913-8981 |
| SLC22A3 | SEQ ID NOs: 8982-9042 |
| SLC25A37 | SEQ ID NOs: 9043-9157 |
| SLC27A5 | SEQ ID NOs: 9158-9196 |
| SMPD1 | SEQ ID NOs: 9197-9223 |
| SNRPB | SEQ ID NOs: 9224-9325 |
| SP140 | SEQ ID NOs: 9326-9379 |
| STAMBP | SEQ ID NOs: 9380-9428 |
| SULF2 | SEQ ID NOs: 9429-9482 |
| SYNJ2 | SEQ ID NOs: 9483-9513 |
| TAZ | SEQ ID NOs: 9514-9554 |
| TDRD7 | SEQ ID NOs: 9555-9586 |
| TFB1M | SEQ ID NOs: 9587-9641 |
| TM6SF1 | SEQ ID NOs: 9642-9671 |
| TOR1B | SEQ ID NOs: 9672-9719 |
| TRAPPC6B | SEQ ID NOs: 9720-9745 |
| TRIP4 | SEQ ID NOs: 9746-9781 |
| TRMU | SEQ ID NOs: 9782-9852 |
| TTC19 | SEQ ID NOs: 9853-9899 |
| TYMP | SEQ ID NOs: 9900-9972 |
| UMPS | SEQ ID NOs: 9973-10032 |
| WDR11 | SEQ ID NOs: 10033-10056 |
| WIPI1 | SEQ ID NOs: 10057-10103 |
| YY1AP1 | SEQ ID NOs: 10104-10193 |
| ZC3H14 | SEQ ID NOs: 10194-10225 |

In some embodiments, an ASO that targets a pre-mRNA disclosed herein is selected from the group consisting of SEQ ID NOs: 4712-10225.

In some embodiments, an ASO that targets a ABCA5 pre-mRNA is selected from the group consisting of SEQ ID NOs: 4712-4748. In some embodiments, an ASO that targets a ABCA7 pre-mRNA is selected from the group consisting of SEQ ID NOs: 4749-4775. In some embodiments, an ASO that targets a ABCD1 pre-mRNA is selected from the group consisting of SEQ ID NOs: 4776-4874. In some embodiments, an ASO that targets a ABR pre-mRNA is selected from the group consisting of SEQ ID NOs: 4875-4971. In some embodiments, an ASO that targets a ACAD9 pre-mRNA is selected from the group consisting of SEQ ID NOs: 4972-5026. In some embodiments, an ASO that targets a ACAP1 pre-mRNA is selected from the group consisting of SEQ ID NOs: 5027-5084. In some embodiments, an ASO that targets a ACTN4 pre-mRNA is selected from the group consisting of SEQ ID NOs: 5085-5174. In some embodiments, an ASO that targets a ADAMTS13 pre-mRNA is selected from the group consisting of SEQ ID NOs: 5175-5209. In some embodiments, an ASO that targets a AKR1E2 pre-mRNA is selected from the group consisting of SEQ ID NOs: 5210-5242. In some embodiments, an ASO that targets a ALG3 pre-mRNA is selected from the group consisting of SEQ ID NOs: 5243-5411. In some embodiments, an ASO that targets a ANKRD29 pre-mRNA is selected from the group consisting of SEQ ID NOs: 5412-5458. In some embodiments, an ASO that targets a AP3M1 pre-mRNA is selected from the group consisting of SEQ ID NOs: 5459-5482. In some embodiments, an ASO that targets a ASAP3 pre-mRNA is selected from the group consisting of SEQ ID NOs: 5483-5527. In some embodiments, an ASO that targets a ATRX pre-mRNA is selected from the group consisting of SEQ ID NOs: 5528-5574. In some embodiments, an ASO that targets a BBS4 pre-mRNA is selected from the group consisting of SEQ ID NOs: 5575-5656. In some embodiments, an ASO that targets a CATSPER1 pre-mRNA is selected from the group consisting of SEQ ID NOs: 5657-5683. In some embodiments, an ASO that targets a CCDC40 pre-mRNA is selected from the group consisting of SEQ ID NOs: 5684-5720. In some embodiments, an ASO that targets a CCDC88B pre-mRNA is selected from the group consisting of SEQ ID NOs: 5721-5765. In some embodiments, an ASO that targets a CD4 pre-mRNA is selected from the group consisting of SEQ ID NOs: 5766-5806. In some embodiments, an ASO that targets a CES2 pre-mRNA is selected from the group consisting of SEQ ID NOs: 5807-5878. In some embodiments, an ASO that targets a COL5A3 pre-mRNA is selected from the group consisting of SEQ ID NOs: 5879-5924. In some embodiments, an ASO that targets a CPSF1 pre-mRNA is selected from the group consisting of SEQ ID NOs: 5925-5954. In some embodiments, an ASO that targets a CSPP1 pre-mRNA is selected from the group consisting of SEQ ID NOs: 5955-5982. In some embodiments, an ASO that targets a CTH pre-mRNA is selected from the group consisting of SEQ ID NOs: 5983-6016. In some embodiments, an ASO that targets a DAB2IP pre-mRNA is selected from the group consisting of SEQ ID NOs: 6017-6063. In some embodiments, an ASO that targets a DBN1 pre-mRNA is selected from the group consisting of SEQ ID NOs: 6064-6105. In some embodiments, an ASO that targets a DDX51 pre-mRNA is selected from the group consisting of SEQ ID NOs: 6106-6152. In some embodiments, an ASO that targets a DEAF1 pre-mRNA is selected from the group consisting of SEQ ID NOs: 6153-6187. In some embodiments, an ASO that targets a DGKE pre-mRNA is selected from the group consisting of SEQ ID NOs: 6188-6214. In some embodiments, an ASO that targets a DMKN pre-mRNA is selected from the group consisting of SEQ ID NOs: 6215-6257. In some embodiments, an ASO that targets a DOCK8 pre-mRNA is selected from the group consisting of SEQ ID NOs: 6258-6354. In some embodiments, an ASO that targets a DOK5 pre-mRNA is selected from the group consisting of SEQ ID NOs: 6355-6408. In some embodiments, an ASO that targets a EIF2B5 pre-mRNA is selected from the group consisting of SEQ ID NOs: 6409-6440. In some embodiments, an ASO that targets a ELAC2 pre-mRNA is selected from the group consisting of SEQ ID NOs: 6441-6487. In some embodiments, an ASO that targets a ELP1 pre-mRNA is selected from the group consisting of SEQ ID NOs: 6488-6519. In some embodiments, an ASO that targets a ETHE1 pre-mRNA is selected from the group consisting of SEQ ID NOs: 6520-6570. In some embodiments, an ASO that targets a FANCA pre-mRNA is selected from the group consisting of SEQ ID NOs: 6571-6647. In some embodiments, an ASO that targets a FASTK pre-mRNA is selected from the group consisting of SEQ ID NOs: 6648-6762. In some embodiments, an ASO that targets a FPGS pre-mRNA is selected from the group consisting of SEQ ID NOs: 6763-6799. In some embodiments, an ASO that targets a GAS8 pre-mRNA is selected from the group consisting of SEQ ID NOs: 6800-6857. In some embodiments, an ASO that targets a GCK pre-mRNA is selected from the group consisting of SEQ ID NOs: 6858-6954. In some embodiments, an ASO that targets a GGA1 pre-mRNA is selected from the group consisting of SEQ ID NOs: 6955-7024. In some embodiments, an ASO that targets a GON4L pre-mRNA is selected from the group consisting of SEQ ID NOs: 7025-7051. In some embodiments, an ASO that targets a HELQ pre-mRNA is selected from the group consisting of SEQ ID NOs: 7052-7096. In some embodiments, an ASO that targets a HPS1 pre-mRNA is selected from the group consisting of SEQ ID NOs: 7097-7124. In some embodiments, an ASO that targets a IL17RC pre-mRNA is selected from the group consisting of SEQ ID NOs: 7125-7162. In some embodiments, an ASO that targets a INVS pre-mRNA is selected from the group consisting of SEQ ID NOs: 7163-7260. In some embodiments, an ASO that targets a IRF3 pre-mRNA is selected from the group consisting of SEQ ID NOs: 7261-7379. In some embodiments, an ASO that targets a KLKB1 pre-mRNA is selected from the group consisting of SEQ ID NOs: 7380-7405. In some embodiments, an ASO that targets a LAMC3 pre-mRNA is selected from the group consisting of SEQ ID NOs: 7406-7454. In some embodiments, an ASO that targets a LDAH pre-mRNA is selected from the group consisting of SEQ ID NOs: 7455-7487. In some embodiments, an ASO that targets a LIMS2 pre-mRNA is selected from the group consisting of SEQ ID NOs: 7488-7585. In some embodiments, an ASO that targets a MALT1 pre-mRNA is selected from the group consisting of SEQ ID NOs: 7586-7641. In some embodiments, an ASO that targets a MAPK13 pre-mRNA is selected from the group consisting of SEQ ID NOs: 7642-7751. In some embodiments, an ASO that targets a MCAT pre-mRNA is selected from the group consisting of SEQ ID NOs: 7752-7777. In some embodiments, an ASO that targets a MCEE pre-mRNA is selected from the group consisting of SEQ ID NOs: 7778-7889. In some embodiments, an ASO that targets a MPI pre-mRNA is selected from the group consisting of SEQ ID NOs: 7890-7931. In some embodiments, an ASO that targets a MSTO1 pre-mRNA is selected from the group consisting of SEQ ID NOs: 7932-7963. In some embodiments, an ASO that targets a NLE1 pre-mRNA is selected from the group consisting of SEQ ID NOs: 7964-7988. In some embodiments, an ASO that targets a NLRC5 pre-mRNA is selected from the group consisting of SEQ ID NOs: 7989-8013. In some embodiments, an ASO that targets a NOM1 pre-mRNA is selected from the group consisting of SEQ ID NOs: 8014-8048. In some embodiments, an ASO that targets a NPR1 pre-mRNA is selected from the group consisting of SEQ ID NOs: 8049-8139. In some embodiments, an ASO that targets a OSGEP pre-mRNA is selected from the group consisting of SEQ ID NOs: 8140-8165. In some embodiments, an ASO that targets a PABPC4 pre-mRNA is selected from the group consisting of SEQ ID NOs: 8166-8212. In some embodiments, an ASO that targets a PCOLCE pre-mRNA is selected from the group consisting of SEQ ID NOs: 8213-8288. In some embodiments, an ASO that targets a PLD2 pre-mRNA is selected from the group consisting of SEQ ID NOs: 8289-8305. In some embodiments, an ASO that targets a PNPO pre-mRNA is selected from the group consisting of SEQ ID NOs: 8306-8372. In some embodiments, an ASO that targets a POLE pre-mRNA is selected from the group consisting of SEQ ID NOs: 8373-8401. In some embodiments, an ASO that targets a PON2 pre-mRNA is selected from the group consisting of SEQ ID NOs: 8402-8434. In some embodiments, an ASO that targets a PYCR1 pre-mRNA is selected from the group consisting of SEQ ID NOs: 8435-8493. In some embodiments, an ASO that targets a RAD52 pre-mRNA is selected from the group consisting of SEQ ID NOs: 8494-8546. In some embodiments, an ASO that targets a RHBDF2 pre-mRNA is selected from the group consisting of SEQ ID NOs: 8547-8579. In some embodiments, an ASO that targets a RMND1 pre-mRNA is selected from the group consisting of SEQ ID NOs: 8580-8604. In some embodiments, an ASO that targets a ROBO3 pre-mRNA is selected from the group consisting of SEQ ID NOs: 8605-8756. In some embodiments, an ASO that targets a RPGRIP1 pre-mRNA is selected from the group consisting of SEQ ID NOs: 8757-8821. In some embodiments, an ASO that targets a RTTN pre-mRNA is selected from the group consisting of SEQ ID NOs: 8822-8855. In some embodiments, an ASO that targets a SIK3 pre-mRNA is selected from the group consisting of SEQ ID NOs: 8856-8912. In some embodiments, an ASO that targets a SLC12A7 pre-mRNA is selected from the group consisting of SEQ ID NOs: 8913-8981. In some embodiments, an ASO that targets a SLC22A3 pre-mRNA is selected from the group consisting of SEQ ID NOs: 8982-9042. In some embodiments, an ASO that targets a SLC25A37 pre-mRNA is selected from the group consisting of SEQ ID NOs: 9043-9157. In some embodiments, an ASO that targets a SLC27A5 pre-mRNA is selected from the group consisting of SEQ ID NOs: 9158-9196. In some embodiments, an ASO that targets a SMPD1 pre-mRNA is selected from the group consisting of SEQ ID NOs: 9197-9223. In some embodiments, an ASO that targets a SNRPB pre-mRNA is selected from the group consisting of SEQ ID NOs: 9224-9325. In some embodiments, an ASO that targets a SP140 pre-mRNA is selected from the group consisting of SEQ ID NOs: 9326-9379. In some embodiments, an ASO that targets a STAMBP pre-mRNA is selected from the group consisting of SEQ ID NOs: 9380-9428. In some embodiments, an ASO that targets a SULF2 pre-mRNA is selected from the group consisting of SEQ ID NOs: 9429-9482. In some embodiments, an ASO that targets a SYNJ2 pre-mRNA is selected from the group consisting of SEQ ID NOs: 9483-9513. In some embodiments, an ASO that targets a TAZ pre-mRNA is selected from the group consisting of SEQ ID NOs: 9514-9554. In some embodiments, an ASO that targets a TDRD7 pre-mRNA is selected from the group consisting of SEQ ID NOs: 9555-9586. In some embodiments, an ASO that targets a TFB1M pre-mRNA is selected from the group consisting of SEQ ID NOs: 9587-9641. In some embodiments, an ASO that targets a TM6SF1 pre-mRNA is selected from the group consisting of SEQ ID NOs: 9642-9671. In some embodiments, an ASO that targets a TOR1B pre-mRNA is selected from the group consisting of SEQ ID NOs: 9672-9719. In some embodiments, an ASO that targets a TRAPPC6B pre-mRNA is selected from the group consisting of SEQ ID NOs: 9720-9745. In some embodiments, an ASO that targets a TRIP4 pre-mRNA is selected from the group consisting of SEQ ID NOs: 9746-9781. In some embodiments, an ASO that targets a TRMU pre-mRNA is selected from the group consisting of SEQ ID NOs: 9782-9852. In some embodiments, an ASO that targets a TTC19 pre-mRNA is selected from the group consisting of SEQ ID NOs: 9853-9899. In some embodiments, an ASO that targets a TYMP pre-mRNA is selected from the group consisting of SEQ ID NOs: 9900-9972. In some embodiments, an ASO that targets a UMPS pre-mRNA is selected from the group consisting of SEQ ID NOs: 9973-10032. In some embodiments, an ASO that targets a WDR11 pre-mRNA is selected from the group consisting of SEQ ID NOs: 10033-10056. In some embodiments, an ASO that targets a WIPI1 pre-mRNA is selected from the group consisting of SEQ ID NOs: 10057-10103. In some embodiments, an ASO that targets a YY1AP1 pre-mRNA is selected from the group consisting of SEQ ID NOs: 10104-10193. In some embodiments, an ASO that targets a ZC3H14 pre-mRNA is selected from the group consisting of SEQ ID NOs: 10194-10225.

### Combinational Therapy

In some embodiments, provided herein is a composition comprising one or more NSAE-modulating agents. In some embodiments, provided herein is a composition comprising two or more NSAE-modulating agents. In some embodiments, provided herein is a composition comprising one or more ASO complementary to a targeted region of SynGAP1 pre-mRNA. In some embodiments, provided herein is a composition comprising two or more ASO complementary to a targeted region of SynGAP1 pre-mRNA. In some embodiments, provided herein is a composition comprising one or more ASO complementary to a same targeted region of SynGAP1 pre-mRNA. In some embodiments, provided herein is a composition comprising two or more ASO complementary to a same targeted region of SynGAP1 pre-mRNA. In some embodiments, provided herein is a composition comprising one or more ASO complementary to different targeted regions of SynGAP1 pre-mRNA. In some embodiments, provided herein is a composition comprising two or more ASO complementary to different targeted regions of SynGAP1 pre-mRNA. In some embodiments, provided herein is a composition comprising one or more ASOs of Table 1. In some embodiments, provided herein is a composition comprising two and more ASOs of in Table 1. In some embodiments, provided herein is a composition comprising one or more ASOs selected from SEQ ID NOs: 1301 to 10225. In some embodiments, provided herein is a composition comprising two and more ASOs selected from SEQ ID NOs: 1301 to 10225.

In some embodiments, provided herein is a composition comprising one or more ASO complementary to a targeted region of a target pre-mRNA, wherein the target is any one selected from the group consisting of ABCA5, ABCA7, ABCC3, ABCC5, ABCC8, ABCD1, ABR, ACAD9, ACAP1, ACOX2, ACSF3, ACTN4, ADAM17, ADAMTS13, AKR1E2, ALAD, ALG3, ANKRD29, ANKS3, ANO4, AP3B2, AP3M1, AP5Z1, ARNTL, ASAP3, ATRX, BBS2, BBS4, BRD9, CALM3, CARS1, CASP9, CATSPER1, CCDC40, CCDC88B, CD4, CD58, CES2, CHFR, CLCN2, CNOT3, CNTROB, COL5A3, CPSF1, CSPP1, CTF1, CTH, CYP3A5, DAB2IP, DBN1, DDX51, DEAF1, DGKE, DHDDS, DMKN, DNHD1, DOCK8, DOK5, EIF2B5, ELAC2, ELP1, ENTPD4, ESRRA, ESS2, ETAA1, ETHE1, FANCA, FASTK, FASTKD3, FPGS, FREM1, GAS8, GCK, GGA1, GGA3, GGT1, GLMN, GON4L, GRB14, HDAC3, HELQ, HP1BP3, HPS1, HPS4, IDUA, IFT122, IL17RC, IMPDH1, INVS, IRF3, KCTD3, KLKB1, KYAT1, LAMC3, LDAH, LIMS2, MALT1, MAP3K7, MAPK13, MCAT, MCEE, MPI, MSTO1, NBEAL2, NLE1, NLRC5, NOM1, NOP58, NPHP1, NPR1, NUP188, OSGEP, PABPC4, PCOLCE, PHKA2, PIDD1, PKD1, PLA2G6, PLD2, PLEKHG5, PNPO, POLE, PON2, PRMT7, PRODH, PRPF3, PRPF4, PYCR1, RAD52, REXO1, RFX5, RHBDF2, RMND1, ROBO3, RPGRIP1, RTTN, RUFY3, SEMA3B, SEMA3F, SH2D3A, SIK3, SIRT3, SKIV2L, SLC2A13, SLC12A7, SLC22A3, SLC25A13, SLC25A37, SLC27A5, SLC30A9, SLC30A10, SMPD1, SMTN, SNRPB, SP140, STAMBP, STXBP2, SULF2, SYNGAP1, SYNJ2, TAZ, TBL2, TDRD7, TECPR2, TFB1M, TM6SF1, TNK2, TOE1, TOR1B, TRAPPC6B, TRIP4, TRMT2A, TRMU, TRPV4, TTC19, TYMP, UMPS, UROD, WDR11, WDR62, WIPI1, WRAP53, XPO1, YY1AP1, and ZC3H14. In some embodiments, provided herein is a composition comprising two or more ASO complementary to a targeted region of a target pre-mRNA. In some embodiments, provided herein is a composition comprising one or more ASO complementary to a same targeted region of a target pre-mRNA. In some embodiments, provided herein is a composition comprising two or more ASO complementary to a same targeted region of a target pre-mRNA. In some embodiments, provided herein is a composition comprising one or more ASO complementary to different targeted regions of a target pre-mRNA. In some embodiments, provided herein is a composition comprising two or more ASO complementary to different targeted regions of a target pre-mRNA. In some embodiments, provided herein is a composition comprising two or more ASO complementary to a targeted region of two or more target pre-mRNAs. In some embodiments, provided herein is a composition comprising two or more ASO complementary to a targeted region of two or more different target pre-mRNAs.

In some aspects, provided herein is a method of modulating expression of a target protein by administering the compositions as described above. In some aspects, provided herein is a pharmaceutical composition comprising a therapeutic agent comprising the composition as described above; and a pharmaceutically acceptable excipient and/or a delivery vehicle. In some aspects, provided herein is a method of treating or preventing a disease or condition in a subject in need thereof, the method comprising: administering to the subject a pharmaceutical composition comprising a therapeutic agent comprising the composition as described above; and a pharmaceutically acceptable excipient and/or a delivery vehicle. In some aspects, provided herein is a method of treating or preventing a disease or condition in a subject in need thereof, the method comprising: administering to the subject the composition as described above. In some aspects, provided herein is a method of treating a disease or condition in a subject in need thereof by modulating expression of a target protein in a cell of the subject through administering to the subject a pharmaceutical composition comprising a therapeutic agent comprising the composition as described above; and a pharmaceutically acceptable excipient and/or a delivery vehicle. In some aspects, provided herein is a method of treating a disease or condition in a subject to the subject in need thereof by modulating expression of a target protein in a cell of the subject through administering to the subject the composition as described above. In some aspects, provided herein is a therapeutic agent for use in a method described herein. In some aspects, provided herein is a pharmaceutical composition comprising a therapeutic agent described herein and a pharmaceutically acceptable excipient.

The present invention will be more specifically illustrated by the following Examples. However, it should be understood that the present invention is not limited by these examples in any manner.

### EXAMPLES

### Example 1: Identification of NMD-Inducing Alternative Splicing Events in Transcripts by RNAseq Using Next Generation Sequencing

Non-productive AS events in organs known to be accessible by ASOs were identified by analyzing 83 publicly available RNA-sequencing (RNA-seq) datasets from human liver, kidney, central nervous system (CNS), and eye tissues. Computational analysis discovered 7,819 unique genes containing a total of 13,121 non-productive AS events of various types. By cross-referencing these genes with genetic disease databases such as Orphanet (www.orpha.net/), 1,265 disease-associated genes with non-productive AS events were identified. As many NMD-sensitive transcripts are efficiently degraded in the analyzed tissues and are not detectable by RNAseq, there are many more genes with non-productive AS events than have been identified to date. To prove the TANGO concept, four genes from the bioinformatic analysis representing three types of targets (haploinsufficiency, autosomal recessive and pathway associated with disease), three organs (liver, CNS, and eye), and three types of alternative splicing events (cassette exon, alternative splice site, and alternative intron) were selected.

### Example 2: Confirmation of Alternative Splicing Via Cycloheximide Treatment

To validate the in-silico predictions and to quantify the abundance of potentially targetable non-productive AS events, cells were treated with cycloheximide (CHX), a translation inhibitor known to inhibit NMD. Expectedly, reverse transcriptase (RT)-PCR analysis showed a consistent increase in the predicted non-productive SYNGAP1 splicing events in various cell lines upon CHX treatment compared to DMSO-treated cells. The increase indicates that these non-productive AS events lead to transcript degradation by NMD.

### Example 3: SYNGAP1 Exon Region ASO Walk

To identify ASOs that can prevent the non-productive AS events, an initial systematic ASO walk was performed in 5-nt steps along the AS event of interest. These ASOs have a uniform phosphorothioate backbone and methoxyethyl at the 2′ ribose position (2′MOE-PS). These modifications were previously shown to allow binding to RNA with high affinity and to confer resistance to both nucleases and RNase H cleavage of the target RNA-ASO complex. RT-PCR analysis from transfected HEK293 cells identified several ASOs that reduce AS in the SYNGAP1 mRNA and increase productive mRNA. The observed increase in SYNGAP1 productive mRNA was confirmed by TaqMan qPCR. The fold change of AS may be plotted vs the increase in productive mRNA (qPCR) to demonstrate that the ASOs are functioning on mechanism. These results strongly suggest that gene expression upregulation can be achieved by preventing non-productive AS with ASOs.

### Example 4: Use of ASO's to Increase Cellular Protein Expression in a Dose-Dependent Manner

As the desirable upregulation level varies among target genes and diseases, selected positive ASO hits from the initial walks were used to determine whether the increase in productive mRNA can be titrated across non-productive AS events. SYNGAP1 ASO-55 was transfected in cells at increasing concentrations to demonstrate dose-dependent upregulation. The concentration was selected based on the potency of the ASO. RT-PCR results showed a dose-dependent decrease of the non-productive alternative 3′ss selection in SYNGAP1 compared to a non-targeting ASO control transfected at the same respective doses. Conversely, a dose-dependent increase in productive mRNA was observed as measured by TaqMan qPCR compared to a non-targeting ASO control. To determine whether the observed upregulation in productive mRNAs translates to a dose-dependent increase in protein levels, SynGAP proteins were measured in extracts from transfected cells with increasing concentrations of targeting ASOs. First, antibodies against SynGAP were validated by short interfering (si)RNA-mediated knockdown of protein expression and western blot analysis. Immunoblotting results of extracts from cells transfected with the selected ASOs showed a dose-dependent increase in SynGAP proteins. The level of protein upregulation resulting from the highest ASO concentrations ranged between 1.6- to 2-fold. A non-targeting ASO control had no significant effect on protein levels. Altogether, the data indicate that ASOs targeting various types of non-productive AS events lead to a titratable increase in productive mRNA resulting in an increase in protein expression. The titratable nature of TANGO ASO-mediated protein upregulation suggests that one could tightly control protein levels and reduce the risk of overexpression. This aspect of the TANGO technology makes it especially suited to address autosomal dominant haploinsufficient diseases.

### Example 5: Validation of TANGO Approach In Vivo

To prove the applicability of the TANGO mechanism in vivo, one may select a positive hit from an ASO walk targeting a non-productive exon inclusion event in SCN1A. The non-productive AS event in the human SYNGAP1 gene also occurs in mice and is highly conserved at the sequence level (data not shown), allowing for testing the human targeting ASO in mice. Similar to other ASOs presented here, gymnotic (free) uptake of increasing concentrations of SYNGAP1 ASO leads to a dose-dependent decrease of AS and an inversely correlated increase in productive mRNA in cells compared to a non-targeting ASO control. To ascertain whether the observed effect of the ASO can be recapitulated in vivo, administer ASO to six mice or PBS to two mice via intracerebroventricular (ICV) injection at postnatal day 2 (P2). RNA and protein can be extracted from the brain of the treated mice 5 days post-injection. RT-PCR analysis can be done to show clear target engagement and a consistent reduction of non-productive exon inclusion in ASO-treated mice compared to the control PBS cohort. This reduction can be effectively translated to a roughly 4-fold increase in productive mRNA measured by TaqMan qPCR. Concomitantly, increases in protein by western blot can be detected using a validated antibody. These data provide in vivo proof of concept of the TANGO approach to upregulate protein expression by leveraging non-productive AS events.

### Example 6: TANGO (Targeted Augmentation of Nuclear Gene Output) for the Treatment of Genetic Diseases

TANGO (Targeted Augmentation of Nuclear Gene Output), a novel technology which exploits antisense-mediated modulation of pre-mRNA splicing was developed to increase protein expression. TANGO prevents naturally-occurring non-productive splicing events that lead to either transcript degradation by nonsense-mediated mRNA decay (NMD) or nuclear retention. By doing so, TANGO increases the generation of productive mRNA, resulting in an increase of full-length, fully-functional protein. Bioinformatic analyses of RNA sequencing (RNAseq) datasets were undertaken to identify non-productive events. Non-productive events were found in more than 50% of protein-coding genes, of which approximately 2,900 are disease-associated. To validate the in-silico predictions, targets (PCCA, SYNGAP1, CD274, and SCN1A) representing various types of NMD-inducing, non-productive alternative splicing (AS) events (cassette exons, alternative splice sites, and alternative introns) were selected and quantified their abundance by treating cells with cycloheximide (CHX), a translation inhibitor that is known to inhibit NMD. RT-PCR analyses of the selected targets was performed and an increase of the non-productive mRNA upon CHX treatment was observed compared to DMSO-treated cells. Antisense oligonucleotides (ASOs) were designed to target the three types of NMD-inducing, non-productive AS events and TANGO ASOs were able to modulate splicing to increase productive mRNA and protein in a dose-dependent manner in vitro. Consistent with the TANGO mechanism, the level of ASO-mediated upregulation was observed to be directly proportional to the abundance of the targeted NMD-inducing event. Moreover, intracerebroventricular injection in wild-type mice of a TANGO ASO targeting a non-productive AS event in SCN1A led to an increase in productive mRNA and NaV1.1 protein in the brain. As TANGO exploits naturally-occurring non-productive AS, this novel approach can be employed to upregulate gene expression from wild-type or hypomorphic alleles, providing a potentially unique strategy to treat genetic diseases. TANGO is being applied to develop treatment for autosomal dominant haploinsufficiency diseases such as genetic epilepsies. TANGO ASOs that increase expression from the wild-type alleles can be used to restore physiological levels of the deficient proteins.

### Example 7: RNA-Seq Processing Workflow

### Transcript Database and Labeling of NMD Junctions:

Annotated transcripts were downloaded from GENCODE (v. 28) and REFSEQ (via UCSC). Each annotated exon-exon junction was labeled as “coding” or “NMD”. Junctions are labeled “NMD” if and only if that junction is exclusively found in transcripts labeled “nonsense_mediated_decay” (GENCODE) or “NR” (REFSEQ).

### RNA-Seq Library Processing:

All RNA-seq samples were aligned to the hg38 genome and a combined transcript database using STAR¹ v2.6.1b to generate splice junction counts.

### Identification and Quantification of Putative NMD-Inducing Splicing Events:

All samples were run through SUPPA2² to define annotated alternative splicing events. Different approaches were then used to label and quantify each type of alternative splicing as follows: Exon inclusion (EI) and exon skipping (ES): The “skipped exon” events were parsed from SUPPA to obtain the inclusion and skipping junctions for each event. If the skipping junction was labeled “NMD”, the event was labeled “ES_NMD”. If either of the inclusion junctions were labeled as “NMD”, the event was labeled “EI_NMD”. Otherwise, the event is labeled “cassette exon.” Inclusion and skipping junction counts were retrieved from the STAR output, and these counts were summed across all events sharing the same alternatively spliced exon.

The final PSI for the inclusion was calculated as:

$$Ψ = \frac{s {j}_{inc}}{s {j}_{inc} + 2 \cdot {sj}_{skip}} .$$

For inclusion events, Ψ_{EI_NMD}=Ψ.
For skipping events, Ψ_{ES_NMD}=1−Ψ.
Alternative 3′ and 5′ splice sites (A3 and A5): The A3 and A5 events were parsed from SUPPA to obtain the junctions corresponding to each alternative event. If either the long or short junction is labeled as “NMD”, an NMD event is reported. If both junctions report NMD it is not reported because there is likely complex splicing in that region. Splice junction counts were retrieved from the STAR output. The PSI is reported as

$${Ψ}_{A \{ 3 , 5 \} \_ NMD} = \frac{s {j}_{N M D}}{s {j}_{coding}} .$$

Alternative intron events (AI): The retained intron events were parsed from SUPPA to obtain the list of alternative intron events. The AI event is labeled NMD if the event junction is labeled NMD. To calculate PSI, the expression level of the exon within which the AI is located was estimated by summing all junctions using its 3′ and 5′ splice sites (and all other parent exons containing the same AI event). Usage of the AI junction will then fall within the range

$$[ 0 , ∼ \frac{s {j}_{{3}^{′}} + s {j}_{{5}^{′}}}{2} ] ,$$

because full use of the AI junction (which results in no intron retention) is achieved with similar counts at the exon junctions and the AI junction (full intron retention has 0 reads for the junction). To calculate Ψ, the junction counts were normalized, such that this range would now be [0,1]:

$${Ψ}_{AI} ∼ \frac{min ( s {j}_{AI} , \frac{s {j}_{{3}^{′}} + s {j}_{{5}^{′}}}{2} )}{\frac{s {j}_{{3}^{′}} + s {j}_{{5}^{′}}}{2}} .$$

### Annotation of Disease Relevance:

Gene-disease association data from Orphadata (http://www.orphadata.org), the publicly available data repository of Orphanet was downloaded. The annotations were extended to cover all gene symbol aliases.

### Example 8: Treatment with Cycloheximide (CHX), Cell Culture, and Transfections

To determine the abundance of the non-productive mRNAs, cells (HEK293: PCCA, SYNGAP1; ARPE19: CD274; ReNCell VM: SCN1A) were incubated with 50 g/ml of CHX (Cell Signaling Technology) dissolved in DMSO for 3 hours.

For PCCA, HEK293 cells were grown in EMEM with 10% FBS and 1×10⁵ cells were seeded in 24-well plate and reverse-transfected with 80 nM ASOs for initial screening or 1, 5, 25 nM of selected ASO using Lipofectamine RNAiMax reagent (Invitrogen) according to manufacturer's instructions. Total RNA was extracted using RNeasy mini kit (Qiagen) 24 hrs post-transfection and cDNA was synthesized with ImProm-II reverse transcriptase (Promega). Total protein was extracted with RIPA buffer (Cell Signaling Technology) 48 hrs post transfection.

For SYNGAP1, HEK293 cells were grown in EMEM with 10% FBS and 7×10⁵ cells were seeded in 6-well plate and reverse-transfected with 30, 60, and 120 nM of antisense oligonucleotide (ASO) using Lipofectamine RNAiMax reagent (Invitrogen) according to manufacturer's instructions. Total RNA was extracted using RNeasy mini kit (Qiagen) 24 hrs post-transfection and cDNA was synthesized with ImProm-II reverse transcriptase (Promega). Total protein was extracted with RIPA buffer (Cell Signaling Technology) 48 hrs post transfection.

For CD274, Huh7 cells were grown in DMEM with 10% FBS and 1×10⁵ cells were seeded in a 12-well plate and reverse transfected with 5, 20, or 80 nM ASO using Lipofectamine RNAiMAX (Invitrogen) according to manufacturer's instructions. For RT-PCR analysis, cells were treated with 50 g/mL of CHX (Cell Signaling Technology) in DMSO for 3 hours 21 hours post transfection. Total RNA was extracted using RNeasy mini kit (Qiagen) 24 hrs post-transfection and cDNA was synthesized with ImProm-II reverse transcriptase (Promega). Total protein was extracted with RIPA buffer (Cell Signaling Technology) 48 hrs post transfection.

For SCN1A, ReNcell VM cells were grown in complete NSC medium containing 20 ng/mL of bFGF and EGF each on laminin coated flasks (2D culture) until reaching ˜90% confluency. The cells were then detached by accutase treatment, washed with PBS and cultured in complete NSC medium in ultra-low attachment surface 24-well polystyrene plate with 3, 8, 20 μM ASO for gymnotic (free) uptake. Total RNA was extracted using RNeasy mini kit (Qiagen) 72 hrs post-ASO addition to media and cDNA was synthesized with ImProm-II reverse transcriptase (Promega).

### Example 9: qPCR and RT-PCR Assays

For expression analysis of the productive mRNA, TaqMan qPCR (Thermo Fisher SC) was performed for PCCA (Hs01120555_m1), SYNGAP1 (Catalog #Hs00405348_m1), Mo-Scn1a (Mm00450583_mH), RPL32 (Hs00851655_g1), Mo-Gapdh (Mm99999915_g1), SYBR green qPCR was performed for human CD274 with forward primer 5′-AATGTGACCAGCACACTGAG-3′ (SEQ ID NO: 1099) and reverse primer 5′-GAATGTCAGTGCTACACCAAGG-3′ (SEQ ID NO: 1100), and probe-based qPCR (custom-designed, IDT) was performed for Hu-SCN1A with forward primer 5′-TGGGTTACTCAGAACTTGGA-3′ (SEQ ID NO: 1101), reverse primer 5′-GCATTCACAACCACCCTC-3′ (SEQ ID NO: 1102), and probe 5′-/56-FAM/CAAATCTCT/ZEN/CAGGACACTAAGAGCTCTGAGAC/3IABkFQ/-3′ (SEQ ID NO: 1103).

For PCCA, PCR analysis to amplify the productive and non-productive mRNAs was performed with forward primer 5′-GACCCCTACAAGTCTTTTGGTTT-3′ (SEQ ID NO: 1104) and reverse primer 5′-ATCACTTCCTGGTTGGATGC-3′ (SEQ ID NO: 1105). The cycling conditions were 30 sec at 95° C. for denaturation, 30 sec at 60° C. for annealing and 60 sec at 72° C. for extension for 30 cycles. The PCR products were separated on 5% polyacrylamide gel and quantified with Multi Gauge software Version 2.3.

For SYNGAP1, PCR analysis to amplify the productive and non-productive mRNAs was performed using forward primer 5′-GACCCTATCAAGTGCACAGC-3′ (SEQ ID NO: 1106) and reverse primer 5′-CTCCTGCATAAGCCCAAAGAG-3′ (SEQ ID NO: 1107). The cycling conditions were 30 sec at 95° C. for denaturation, 30 sec at 60° C. for annealing, and 60 sec at 72° C. for extension for 32 cycles. The PCR products were separated on a 5% polyacrylamide gel and quantified with Multi Gauge software Version 2.3.

For CD274, PCR analysis to amplify the productive and non-productive mRNAs was performed using forward primer 5′-GTCATCTGGACAAGCAGTG-3′ (SEQ ID NO: 1108) and a reverse primer 5′-GGATGCCACATTTTTTCACATC-3′ (SEQ ID NO: 1109). The cycling conditions were 30 sec at 95° C. for denaturation, 30 sec at 55° C. for annealing, and 30 sec at 72° C. for extension for 29 cycles. The PCR products were separated on a 5% polyacrylamide gel and quantified with Multi Gauge software Version 2.3.

For Hu-SCN1A, PCR analysis to amplify the productive and non-productive mRNAs was performed with forward primer 5′-ATTGTTGATGTTTCATTGGTCAGTTTAACA-3′ (SEQ ID NO: 1110) and reverse primer 5′-GAAGAAGGACCCAAAGATGATGAAAATA-3′ (SEQ ID NO: 1111). The cycling conditions were 30 sec at 95° C. for denaturation, 30 sec at 55° C. for annealing, and 75 sec at 72° C. for extension for 28 cycles. For Mo-Scn1a, PCR analysis to amplify the productive and non-productive mRNAs was performed with forward primer 5′-CAGTTTAACAGCAAATGCCTTGGGTT-3′ (SEQ ID NO: 1112) and reverse primer 5′-AAGTACAAATACATGTACAGGCTTTCCTCATACTTA-3′ (SEQ ID NO: 1113). The cycling conditions were 30 sec at 95° C. for denaturation, 30 sec at 56° C. for annealing, and 75 sec at 72° C. for extension for 28 cycles. For Mo-Gapdh, PCR was performed with forward primer 5′-AGGTCGGTGTGAACGGATTTG-3′ (SEQ ID NO: 1114) and reverse primer 5′-GGGGTCGTTGATGGCAACA-3′ (SEQ ID NO: 1115). The cycling conditions were 30 sec at 95° C. for denaturation, 30 sec at 56° C. for annealing, and 60 sec at 72° C. for extension for 24 cycles. The PCR products were separated on a 5% polyacrylamide gel and quantified with Multi Gauge software Version 2.3.

### Example 10: Western Blotting

Protein extracts were quantified by colorimetric assay using Pierce BCA protein assay kit (ThermoFisher).

For PCCA, immunoblotting was carried out with 25 μg of lysate. Anti-PCCA primary antibody (Cat #ab187686, 1:1000 dilution, overnight at 4° C.) and secondary antibody (1:2000, anti-rabbit IgG Alexa Fluror647, 1 hr at RT) were purchased from Abcam. Blots were scanned using Typhoon RLA 9000 imager (General Electric). Densitometric analysis was carried out using Multi Gauge software Version 2.3.

For SynGAP, immunoblotting was carried out with 60 μg of lysate. Anti-SynGAP primary antibody (Cat #5539, 1:1000, overnight at 4° C.) and secondary anti-rabbit HRP-conjugated (Cat #7074, 1:5000, 1 hr at RT) were purchased from Cell Signaling Technology. Blots were scanned using Typhoon RLA 9000 imager (General Electric). Densitometric analysis was carried out using Multi Gauge software Version 2.3.

For Nav1.1, immunoblotting was carried out with 120 μg of lysate. Anti-Nav1.1 primary antibody (ASC-001, 1:2000, overnight at 4° C.) was purchase from Almone. For vinculin, immunoblotting was carried out with 30 μg of lysate. Anti-vinculin primary E1E9V XP antibody (Cat #13901T, 1:2000, overnight at 4° C.) was purchased from Cell Signaling Technology. Secondary anti-rabbit HRP-conjugated antibody (Cat #sc-2004, 1:2500, overnight at 4° C.) was purchased from Santa Cruz. Blots were scanned using Typhoon RLA 9000 imager (General Electric). Densitometric analysis was carried out using Multi Gauge software Version 2.3.

### Example 11: Flow Cytometry

Cells were lifted from culture plates in FACS buffer (ThermoFisher SCIENTIFIC). Cells were stained with APC-anti-PD-L1 (1:250, BioLegend, #329708). Data from 15,000 cells were collected on a Guava Easycyte 12HT (EMD Millipore) flow cytometer. Fluorescence minus one was used to determine the positive gate.

### Example 12: Single Bolus ICV Injection in Neonate Mouse

Postnatal day 2 (P2) mice were injected with the selected 20 μg ASO or PBS. Pups were immobilized by gently holding them down on a soft tissue padded surface with two fingers. A 33-gauge needle (1-cm long, point style 4, 120 beveled) attached to a 5 L Hamilton microsyringe was used for the injection. The coordinates of the injection were approximately 1 mm lateral from the sagittal suture, halfway between the lambda and bregma, and −2 mm ventral. 2 μL of ASO or PBS was injected slowly into one cerebral lateral ventricle. Injected pups were quickly returned to their nest and were observed daily for survival and signs of stress.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-100. (canceled)

101. A method of modulating expression of a target protein by a cell having a pre-processed mRNA transcript (pre-mRNA) that encodes the target protein, wherein the pre-mRNA comprises:
a non-sense mediated RNA decay-inducing alternative exon (NSAE) comprising an alternative 3′ splice site upstream of the 3′ splice site of a canonical exon and within the canonical exon, or downstream of the 3′ splice site of the canonical exon and within an intron,
the method comprising contacting a NSAE-modulating agent to the cell, wherein the NSAE-modulating agent modulates processing of an mRNA transcript from the pre-mRNA transcript by modulating splicing of the pre-mRNA at the 3′ alternative splice site, and wherein the splicing of the pre-mRNA at the 3′ alternative splice site modulates the expression of the target protein.

102. The method of claim 101, wherein the target protein is selected from the group consisting of ABCA5, ABCA7, ABCD1, ABR, ACAD9, ACAP1, ACTN4, ADAMTS13, AKR1E2, ALG3, ANKRD29, AP3M1, ASAP3, ATRX, BBS4, CATSPER1, CCDC40, CCDC88B, CD4, CES2, COL5A3, CPSF1, CSPP1, CTH, DAB2IP, DBN1, DDX51, DEAF1, DGKE, DMKN, DOCK8, DOK5, EIF2B5, ELAC2, ELP1, ETHE1, FANCA, FASTK, FPGS, GAS8, GCK, GGA1, GON4L, HELQ, HPS1, IL17RC, INVS, IRF3, KLKB1, LAMC3, LDAH, LIMS2, MALT1, MAPK13, MCAT, MCEE, MPI, MSTO1, NLE1, NLRC5, NOM1, NPR1, OSGEP, PABPC4, PCOLCE, PLD2, PNPO, POLE, PON2, PYCR1, RAD52, RHBDF2, RMND1, ROBO3, RPGRIP1, RTTN, SIK3, SLC12A7, SLC22A3, SLC25A37, SLC27A5, SMPD1, SNRPB, SP140, STAMBP, SULF2, SYNJ2, TAZ, TDRD7, TFB1M, TM6SF1, TOR1B, TRAPPC6B, TRIP4, TRMU, TTC19, TYMP, UMPS, WDR11, WIPI1, YY1AP1 and ZC3H14.

103. The method of claim 101, wherein the splicing of the pre-mRNA at the 3′ alternative splice site increases the expression of the target protein in the cell.

104. The method of claim 101, wherein the NSAE comprises an alternative 3′ splice site upstream of the 3′ splice site of a canonical exon and within the canonical exon.

105. The method of claim 101, wherein the NSAE comprises an alternative 3′ splice site downstream of the 3′ splice site of the canonical exon and within the intron.

106. The method of claim 101, wherein the agent is a small molecule or a polypeptide.

107. The method of claim 101, wherein the agent is a nucleic acid binding protein.

108. The method of claim 107, wherein the nucleic acid binding protein contains a TAL-effector, zinc finger binding domain, a Cas family protein.

109. The method of claim 101, wherein the NSAE-modulating agent binds to a targeted region of the pre-processed mRNA transcript.

110. The method of claim 101, wherein the NSAE-modulating agent:
(a) binds to a factor involved in splicing of the NSAE;
(b) inhibits activity of a factor involved in splicing of the NSAE; or
(c) with binding of a factor involved in splicing of the NSAE to a region of the targeted portion of the pre-processed mRNA transcript.

111. The method of claim 109, wherein the agent is an antisense oligomer (ASO) complementary to the targeted region of the pre-mRNA, or a vector encoding the antisense oligomer.

112. The method of claim 111, wherein the antisense oligomer is at least 75% complementary to the targeted region of the pre-mRNA encoding the target protein.

113. The method of claim 111, wherein the agent is the antisense oligomer, and wherein the antisense oligomer comprises a backbone modification comprising a phosphorothioate linkage or a phosphorodiamidate linkage.

114. The method of claim 111, wherein the agent is the antisense oligomer, and wherein the antisense oligomer comprises a phosphorodiamidate morpholino, a locked nucleic acid, or a peptide nucleic acid.

115. The method of claim 111, wherein the agent is the antisense oligomer, and wherein the antisense oligomer comprises a 2′-O-methyl moiety, a 2′-Fluoro moiety, or a 2′-O-methoxyethyl moiety.

116. The method of claim 111, wherein the agent is the antisense oligomer, and wherein the antisense oligomer comprises at least one modified sugar moiety.

117. The method of claim 111, wherein the antisense oligomer consists of from 8 to 50 nucleobases.

118. The method of claim 101, wherein target protein is SYNGAP1 protein, and wherein the pre-mRNA comprises a sequence selected from the group consisting of premma_ENST00000293748.9, premma_ENST00000418600.7, premrna_ENST00000428982.4, premma_ENST00000449372.7, premma_ENST00000470232.1, premrna_ENST00000479510.2, premma_ENST00000628646.2, premma_ENST00000629380.3, premrna_ENST00000635885.1, premma_ENST00000636075.1, premma_ENST00000636116.1, premrna_ENST00000636146.1, premma_ENST00000636193.1, premma_ENST00000636436.1, premrna_ENST00000636443.1, premma_ENST00000636640.1, premma_ENST00000636731.1, premrna_ENST00000636905.1, premma_ENST00000637052.1, premma_ENST00000637194.1, premrna_ENST00000637490.1, premma_ENST00000637587.1, premma_ENST00000637671.1, premrna_ENST00000637721.1, premma_ENST00000637911.1, premma_ENST00000638127.1, premrna_ENST00000638142.2, premma_ENST00000644458.1, premma_ENST00000645250.1, and premma_ENST00000646630.1.

119. The method of claim 109, wherein target protein is SYNGAP1 protein, and wherein the targeted region of the pre-mRNA comprises a sequence with at least 80%, 85%, 90%, 95%, 97%, or 100% sequence identity to a region comprising at least 8 contiguous nucleic acids of a sequence selected from the group consisting of SEQ ID NOs: 166-169 and 980-983, and complements thereof.

120. The method of claim 109, wherein target protein is SYNGAP1 protein, and wherein the targeted region of the SYNGAP1 pre-mRNA comprises a sequence that is complementary to at least 8, 9, 10, 11, 12, 13, 14, 15, 16, 17, 18, 19 or 20 contiguous nucleic acids of a sequence selected from the group consisting of SEQ ID NOs: 1-165 and 4300-4352, and a complement thereof.

121. The method of claim 101, wherein the agent comprises a sequence that comprises has at least about 80% sequence identity to a sequence selected from the group consisting of SEQ ID NOs: 1-143 and 4300-4352.

122. A method of modulating expression of a target protein by a cell having a pre-processed mRNA transcript (pre-mRNA) that encodes the target protein, wherein the pre-mRNA comprises:
a non-sense mediated RNA decay-inducing alternative exon (NSAE) comprising an alternative 5′ splice site downstream of the 5′ splice site of a canonical exon and within the canonical exon, or upstream of the 5′ splice site of the canonical exon and within an intron;
the method comprising contacting a NSAE-modulating agent to the cell, wherein the NSAE-modulating agent modulates processing of an mRNA transcript from the pre-mRNA transcript by modulating splicing of the pre-mRNA at the 5′ alternative splice site, and wherein the splicing of the pre-mRNA at the 5′ alternative splice site modulates the expression of the target protein.

123. A method of treating or preventing a disease or condition in a subject in need thereof, the method comprising: administering to the subject a pharmaceutical composition comprising:
(a) a non-sense mediated RNA decay-inducing alternative exon (NSAE)-modulating agent that modulates expression of a target protein in a cell comprising a pre-processed mRNA transcript (pre-mRNA) that encodes the target protein, wherein the pre-mRNA comprises:
(1) a non-sense mediated RNA decay-inducing alternative exon (NSAE) comprising an alternative 5′ splice site downstream of the 5′ splice site of a canonical exon and within the canonical exon, or upstream of the 5′ splice site of the canonical exon and within an intron;
wherein the NSAE-modulating agent modulates processing of an mRNA transcript from the pre-processed mRNA transcript by modulating splicing of the pre-mRNA at the 5′ alternative splice site, wherein the splicing of the pre-mRNA at the 5′ alternative splice site modulates the expression of the target protein in the cell; or
(2) a non-sense mediated RNA decay-inducing alternative exon (NSAE) comprising an alternative 3′ splice site upstream of the 3′ splice site of a canonical exon and within the canonical exon, or downstream of the 3′ splice site of the canonical exon and within an intron,
wherein the NSAE-modulating agent modulates processing of an mRNA transcript from the pre-processed mRNA transcript by modulating splicing of the pre-mRNA at the 3′ alternative splice site, and wherein the splicing of the pre-mRNA at the 3′ alternative splice site modulates the expression of the target protein in the cell; and
(b) a pharmaceutically acceptable excipient and/or a delivery vehicle,
wherein the disease or condition is treated or prevented in the subject by the administration of the NSAE-modulating agent by a modulation in expression of the target protein.

124. A composition comprising a non-sense mediated RNA decay-inducing alternative exon (NSAE)-modulating agent that modulates expression of a target protein in a cell comprising a pre-processed mRNA transcript (pre-mRNA) that encodes the target protein, wherein the pre-mRNA comprises:
(1) a non-sense mediated RNA decay-inducing alternative exon (NSAE) comprising an alternative 5′ splice site downstream of the 5′ splice site of a canonical exon and within the canonical exon, or upstream of the 5′ splice site of the canonical exon and within an intron,
wherein the NSAE-modulating agent modulates processing of an mRNA transcript from the pre-processed mRNA transcript by modulating splicing of the pre-mRNA at the 5′ alternative splice site, wherein the splicing of the pre-mRNA at the 5′ alternative splice site modulates the expression of the target protein in the cell; or
(2) a non-sense mediated RNA decay-inducing alternative exon (NSAE) comprising an alternative 3′ splice site upstream of the 3′ splice site of a canonical exon and within the canonical exon, or downstream of the 3′ splice site of the canonical exon and within an intron,
wherein the NSAE-modulating agent modulates processing of an mRNA transcript from the pre-processed mRNA transcript by modulating splicing of the pre-mRNA at the 3′ alternative splice site, and wherein the splicing of the pre-mRNA at the 3′ alternative splice site modulates the expression of the target protein in the cell.

125. A composition comprising a modified antisense oligonucleotide sequence or a vector encoding a polynucleotide comprising an antisense oligonucleotide sequence, wherein the antisense oligomer sequence has at least about 80% sequence identity to a sequence selected from the group consisting of SEQ ID NOs: 1-165 and 1301-10225.
